(19) United States
(12) Patent Application Publication
(10) Pub. No.: US 2025/0235460 A1
(43) Pub. Date: Jul. 24, 2025

(54) COMBINATION THERAPY USING GLUCOSE-DEPENDENT INSULINOTROPIC POLYPEPTIDE RECEPTOR ANTAGONIST COMPOUNDS AND GLP-1 RECEPTOR AGONIST COMPOUNDS

(71) Applicant: Pfizer Inc., New York, NY (US)

(72) Inventors: Robert Gregory Dullea, Groton, MA (US); Kevin James Filipski, Reading, MA (US); Matthew Forrest Sammons, Quincy, MA (US)

(73) Assignee: Pfizer Inc., New York, NY (US)

(21) Appl. No.: 19/034,701

(22) Filed: Jan. 23, 2025

Related U.S. Application Data
(60) Provisional application No. 63/624,735, filed on Jan. 24, 2024.
(60) Provisional application No. 63/624,737, filed on Jan. 24, 2024.
(60) Provisional application No. 63/637,986, filed on Apr. 24, 2024.
(60) Provisional application No. 63/734,355, filed on Dec. 16, 2024.

(51) Int. Cl. A61K 31/5377; A61K 9/00; A61K 31/404; A61K 31/438; A61K 31/4439; A61K 31/444; A61K 31/4709; A61K 31/496; A61K 31/503; A61K 31/506; A61P 3/04

(52) U.S. Cl. CPC A61K 31/5377; A61K 9/0019; A61K 9/0053; A61K 31/404; A61K 31/438; A61K 31/4439; A61K 31/444; A61K 31/4709; A61K 31/496; A61K 31/503; A61K 31/506; A61P 3/04

(57) ABSTRACT

Described herein is a method of treating a disease or condition, for example, obesity, weight gain, or diabetes, comprising administering to a subject in need thereof a glucose-dependent insulinotropic polypeptide receptor (GIPR) antagonist and a glucagon-like peptide 1 receptor (GLP-1R) agonist. Further described herein are pharmaceutical compositions comprising a GIPR antagonist and a GLP-1R agonist, which may be useful in the treatment of a disease or condition, for example, obesity, weight gain, or diabetes.

### BACKGROUND OF THE INVENTION

Glucose-dependent insulinotropic polypeptide (GIP) is a 42-amino acid peptide secreted from K-cells in the small intestine, and GIP secretion is induced by food ingestion. GIP is a known insulinotropic factor that enhances glucose-dependent insulin secretion. GIP has additional physiological effects in multiple tissues, including the promotion of fat storage in the adipose. Upon stimulation with GIP, GIP receptor (GIPR) undergoes structural changes from an inactive conformation to an active conformation, thereby triggering a Gas-mediated increase in cAMP production. GIPR inhibition may be used as a therapeutic intervention for obesity and metabolic diseases.

### SUMMARY OF THE INVENTION

The present invention provides, in part, a method of treating a disease or condition by administering to a subject in need thereof a first therapeutically effective amount of a GIPR antagonist compound of the disclosure or a pharmaceutically acceptable salt thereof, and a second therapeutically effective amount of a glucagon-like peptide 1 receptor (GLP-1R) agonist compound of the disclosure or a pharmaceutically acceptable salt thereof. The GIPR antagonist compound may antagonize the activity of GIPR, and the GLP-1R agonist compound may agonize the activity of GLP-1R, the combination of which may be useful in the treatment of a disease or condition selected from the group consisting of: diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

Disclosed herein is a method of treating a disease or condition comprising administering to a subject in need thereof a therapeutically effective amount of: a) a glucose-dependent insulinotropic polypeptide receptor (GIPR) antagonist small molecule compound or a pharmaceutically acceptable salt thereof; and b) a glucagon-like peptide 1 receptor (GLP-1R) agonist small molecule compound or a pharmaceutically acceptable salt thereof, wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Disclosed herein is a method of treating a disease or condition comprising administering to a subject in need thereof a therapeutically effective amount of:
a) a GIPR antagonist small molecule compound of Formula I:

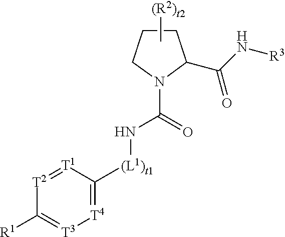

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

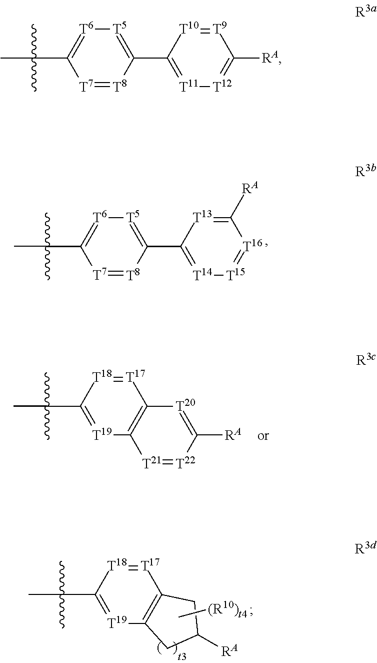

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound or a pharmaceutically acceptable salt thereof,
wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Further disclosed herein is a pharmaceutical composition comprising:
a) a GIPR antagonist small molecule compound of Formula I:

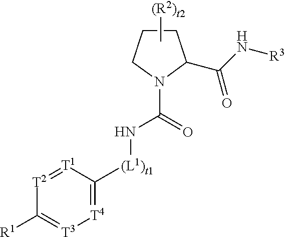

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

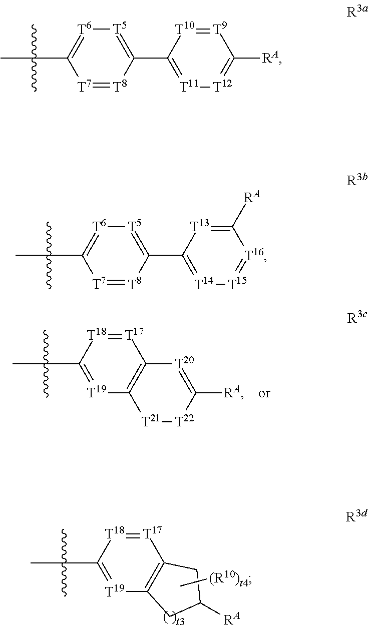

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula B-I:

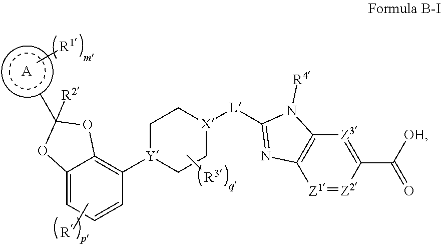

or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O′}, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}′, and —N(R^{N}′)₂;
R^{5′} is a 4- to 6-membered heterocycloalkyl, wherein said heterocycloalkyl may be substituted with 0 to 2 substituents as valency allows independently selected from:
   0 to 1 oxo (═O),
   0 to 1 —CN,
   0 to 2 F atoms, and
   0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O}′;
R^{6′} is a 5- to 6-membered heteroaryl, wherein said heteroaryl may be substituted with 0 to 2 substituents as valency allows independently selected from:
   0 to 2 halogens,
   0 to 1 substituent selected from —OR^{O}′ and —N(R^{N}′)₂, and
   0 to 2 —C₁₋₃alkyl, wherein the alkyl may be substituted with 0 to 3 substituents as valency allows independently selected from:
   0 to 3 F atoms, and
   0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z′}, or one of Z¹′, Z²′, and Z³′ is N and the other two are —CR^{Z}′; and
each R^{Z}′ is independently H, F, Cl, or —CH₃.

Further disclosed herein is a pharmaceutical composition comprising:
a) a GIPR antagonist small molecule compound of Formula I:

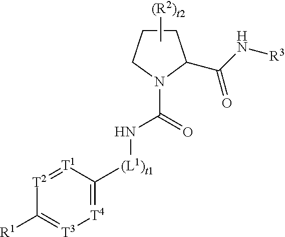

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

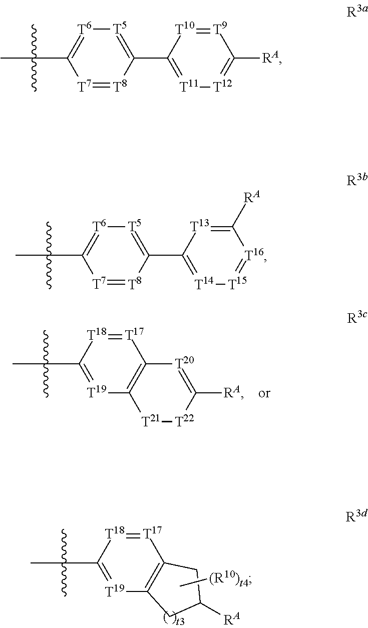

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹ and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula C-I:

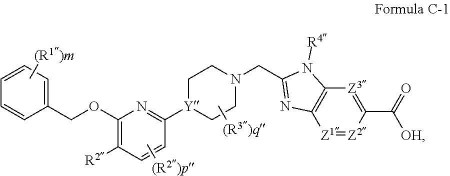

or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and
wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{5″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
   0 to 3 F atoms, and
   0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
each R^{Z″} is independently H, F, Cl, or —CH₃.

It is to be understood that both the foregoing general description and the following detailed description are exemplary and explanatory only and are not restrictive of the invention, as claimed.

### BRIEF DESCRIPTION OF THE DRAWINGS

FIG. 1 shows the percent body weight change from baseline for hGIPR mice on a high fat diet treated with Compound 1 and liraglutide.

FIG. 2 shows the percent body weight change from baseline for hGIPR mice on a high fat diet treated with Compound X and liraglutide.

### DETAILED DESCRIPTION OF THE INVENTION

Glucose-dependent insulinotropic polypeptide (GIP, formerly called gastric inhibitory polypeptide) is a 42-amino acid peptide secreted from K-cells in the small intestine (e.g., duodenum and jejunum), and GIP secretion is induced by food ingestion. GIP is a known insulinotropic factor (or “incretin”) that enhances glucose-dependent insulin secretion. GIP has additional physiological effects in multiple tissues, including the promotion of fat storage in the adipose. Intact GIP is rapidly inactivated by dipeptidyl peptidase 4 (DPPIV).

The GIP receptor (GIPR) belongs to the glucagon subfamily of class 1 G protein-coupled receptors (GPCRs), which is characterized by an extracellular N-terminal domain, seven transmembrane domains, and an intracellular C-terminus (e.g., Zhao et al. Nat Commun. 2022, 13:1057). The N-terminal extracellular domain forms the primary peptide recognition and binding site of the receptor. Upon stimulation with GIP, GIPR undergoes structural changes from an inactive conformation to an active conformation, thereby triggering a Gas-mediated increase in cAMP production. GIPR is expressed in various tissues, including the pancreas, gut, adipose tissue, vasculature, heart, and brain (e.g., Hammoud et al. Nat Rev Endocrinol 2023; 18: 201-216). Human GIPR comprises 466 amino acids and is encoded by a gene located on chromosome 19 (e.g., Gremlich et al., Diabetes. 1995; 44:1202-8; and Volz et al., FEBS Lett. 1995, 373:23-29).

GIPR knockout mice are resistant to high fat diet-induced weight gain and demonstrate improved insulin sensitivity and lipid profiles (e.g., Yamada et al. Diabetes. 2006, 55:S86; and Miyawaki et al. Nature Med. 2002, 8:738-742). Heterozygous loss of function in GIPR has been shown to lower BMI and obesity risk in humans (e.g., Akbari et al. Science. 2021, 373: 6550). Small molecules, peptides, and monoclonal antibodies with antagonist activity at GIPR have been shown to prevent weight gain and insulin resistance in preclinical obesity models (e.g., Nakamura et al. Diabetes Metab Syndr Obes. 2021,14:1095-1105; Yang et al. Mol Metab. 2022, 66: 101638; and Killion et al. 2018). Moreover, human epicardial adipose tissue—which plays a crucial role in the development and progression of coronary artery disease, atrial fibrillation, and heart failure—has been found to express GIPR genes and proteins (e.g., Malavazos et al., European Journal of Preventive Cardiology (2023) 00, 1-14). Collectively, these data suggest that GIPR inhibition may be used as a therapeutic intervention for obesity and metabolic diseases.

Treatment with a combination of a GIPR antagonist with a GLP-1R agonist has been associated with superior weight loss in mice (e.g., Lu et al. Cell Rep Med. 2021, 2(5):100263). Thus, combination therapy with a GIPR antagonist and a GLP-1R agonist may be more effective in treating obesity and metabolic diseases as compared to treatment with a GIPR antagonist alone. Disclosed herein are novel GIPR antagonist compounds with improved pharmaceutical properties (e.g., efficacy, selectivity, reduced toxicity, improved patient compliance, improved biopharmaceutical properties, such as physical stability, solubility, oral availability, metabolic stability, clearance, half-life). Further disclosed herein are novel combinations of GIPR antagonist compounds and GLP-1R agonist compounds that can be used to treat or prevent GIPR-related conditions, diseases, or disorders described herein.

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

### Definitions

Unless otherwise defined herein, scientific and technical terms used in connection with the present invention have the meanings that are commonly understood by those of ordinary skill in the art. The invention described herein suitably may be practiced in the absence of any element(s) not specifically disclosed herein.

“Compounds of the invention” include compound disclosed herein and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter means that the parameter may vary by as much as 10% below or above the stated numerical value for that parameter. For example, a dose of about 5 mg means 5%±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

As used herein, a wavy line,

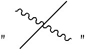

denotes a point of attachment of a substituent to another group.

As used herein, the term “n-membered”, where n is an integer, typically describes the number of ring-forming atoms in a moiety where the number of ring-forming atoms is n. For example, piperidinyl is an example of a 6-membered heterocycloalkyl ring and pyrrolindinyl is an example of a 5-membered heterocycloalkyl group.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₈alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁-C₆ haloalkyl”), 1-4 carbon atoms (“C₁-C₄ haloalkyl”), or 1-2 carbon atoms (“C₁-C₂ haloalkyl”). More specifically, fluorinated alkyl groups may be specifically referred to as “fluoroalkyl.”

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Haloalkoxy” refers to an alkoxyl group as defined above containing the specified number of carbon atoms wherein at least one hydrogen atom has been replaced by halogen. Haloalkoxy groups may contain, but are not limited to, 1-6 carbon atoms, (“C₁-C₆ haloalkoxy”), 1-4 carbon atoms (“C₁-C₄ haloalkoxy”), or 1-2 carbon atoms (“C₁-C₂ haloalkoxy”). More specifically, fluorinated alkoxyl groups may be specifically referred to as “fluoroalkoxy.” “Alkoxyalkyl” refers to an alkyl group, as defined herein, that is substituted by an alkoxy group, as defined herein. Examples include, but are not limited to, CH₃OCH₂— and CH₃CH₂OCH₂—.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring.

Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicycle[1.1.1]pentyl. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O— or OC₁_cycloalkyl. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicycle[1.1.1]pentyl.

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted, unsubstituted or substituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocycloalkyl groups, for example 4-7 or 4-6 membered heterocycloalkyl groups, in accordance with the definition herein. Illustrative examples of heterocycloalkyl rings include, but are not limited to a monovalent radical of oxirane (oxiranyl), thiirane (thiiranyl), aziridine (aziridinyl), oxetane (oxetanyl), thietane (thietanyl), azetidine (azetidinyl), tetrahydrofuran (tetrahydrofuranyl), tetrahydrothiophene (tetrahydrothiophenyl), pyrrolidine (pyrrolidinyl), tetrahydropyran (tetrahydropyranyl), tetrahydrothiopyran (tetrahydrothiopyranyl), piperidine (piperidinyl), 1,4-dioxane (1,4-dioxanyl), 1,4-oxathiarane (1,4-oxathiaranyl), morpholine (morpholinyl), 1,4-dithiane (1,4-dithianyl), piperazine (piperazinyl), thiomorpholine (thiomorpholinyl), oxepane (oxepanyl), thiepane (thiepanyl), azepane (azepanyl), 1,4-dioxepane (1,4-dioxepanyl), 1,4-oxathiepane (1,4-oxathiepanyl), 1,4-oxaazepane (1,4-oxaazepanyl), 1,4-thiazepane (1,4-thiazapanyl), 1,4-diazepane (1,4-diazepanyl), or 1,4-dithepane (1,4-dithiepanyl). Illustrative examples of bridged and fused heterocycloalkyl groups include, but are not limited to a monovalent radical of 1-oxa-5-azabicyclo-[2.2.1]heptane, 3-oxa-8-azabicyclo-[3.2.1]octane, 3-azabicyclo-[3.1.0]hexane, or 2-azabicyclo-[3.1.0]hexane.

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

Illustrative examples of monocyclic heteroaryl groups include, but are not limited to a monovalent radical of pyrrole (pyrrolyl), furan (furanyl), thiophene (thiophenyl), pyrazole (pyrazolyl), imidazole (imidazolyl), isoxazole (isoxazolyl), oxazole (oxazolyl), isothiazole (isothiazolyl), thiazolyl (thiazolyl), 1,2,3-triazole (1,2,3-triazolyl), 1,3,4-triazole (1,3,4-triazolyl), 1-oxa-2,3-diazole (1-oxa-2,3-diazolyl), 1-oxa-2,4-diazole (1-oxa-2,4-diazolyl), 1-oxa-2,5-diazole (1-oxa-2,5-diazolyl), 1-oxa-3,4-diazole (1-oxa-3,4-diazolyl), 1-thia-2,3-diazole (1-thia-2,3-diazolyl), 1-thia-2,4-diazole (1-thia-2,4-diazolyl), 1-thia-2,5-diazole (1-thia-2,5-diazolyl), 1-thia-3,4-diazole (1-thia-3,4-diazolyl), tetrazole(tetrazolyl), pyridine (pyridinyl), pyridazine (pyridazinyl), pyrimidine (pyrimidinyl), or pyrazine (pyrazinyl).

Illustrative examples of fused ring heteroaryl groups include, but are not limited to benzofuran (benzofuranyl), benzothiophene (benzothiophenyl), indole (indolyl), benzimidazole (benzimidazolyl), indazole (indazolyl), benzotriazole (benzotriazolyl), pyrrolo[2,3-b]pyridine (pyrrolo[2,3-b]pyridinyl), pyrrolo[2,3-c]pyridine (pyrrolo[2,3-c]pyridinyl), pyrrolo[3,2-c]pyridine (pyrrolo[3,2-c]pyridinyl), pyrrolo[3,2-b]pyridine (pyrrolo[3,2-b]pyridinyl), imidazo[4,5-b]pyridine (imidazo[4,5-b]pyridinyl), imidazo[4,5-c]pyridine (imidazo[4,5-c]pyridinyl), pyrazolo[4,3-d]pyridine (pyrazolo[4,3-d]pyridinyl), pyrazolo[4,3-c]pyridine (pyrazolo[4,3-c]pyridinyl), pyrazolo[3,4-c]pyridine (pyrazolo[3,4-c]pyridinyl), pyrazolo[3,4-b]pyridine (pyrazolo[3,4-b]pyridinyl), isoindole (isoindolyl), indazole (indazolyl), purine (purinyl), indolizine (indolizinyl), imidazo[1,2-a]pyridine (imidazo[1,2-a]pyridinyl), imidazo[1,5-a]pyridine (imidazo[1,5-a]pyridinyl), pyrazolo[1,5-a]pyridine (pyrazolo[1,5-a]pyridinyl), pyrrolo[1,2-b]pyridazine (pyrrolo[1,2-b]pyridazinyl), imidazo[1,2-c]pyrimidine (imidazo[1,2-c]pyrimidinyl), quinoline (quinolinyl), isoquinoline (isoquinolinyl), cinnoline (cinnolinyl), quinazoline (azaquinazoline), quinoxaline (quinoxalinyl), phthalazine (phthalazinyl), 1,6-naphthyridine (1,6-naphthyridinyl), 1,7-naphthyridine (1,7-naphthyridinyl), 1,8-naphthyridine (1,8-naphthyridinyl), 1,5-naphthyridine (1,5-naphthyridinyl), 2,6-naphthyridine (2,6-naphthyridinyl), 2,7-naphthyridine (2,7-naphthyridinyl), pyrido[3,2-d]pyrimidine (pyrido[3,2-d]pyrimidinyl), pyrido[4,3-d]pyrimidine (pyrido[4,3-d]pyrimidinyl), pyrido[3,4-d]pyrimidine (pyrido[3,4-d]pyrimidinyl), pyrido[2,3-d]pyrimidine (pyrido[2,3-d]pyrimidinyl), pyrido[2,3-b]pyrazine (pyrido[2,3-b]pyrazinyl), pyrido[3,4-b]pyrazine (pyrido[3,4-b]pyrazinyl), pyrimido[5,4-d]pyrimidine (pyrimido[5,4-d]pyrimidinyl), pyrazino[2,3-b]pyrazine (pyrazino[2,3-b]pyrazinyl), or pyrimido[4,5-d]pyrimidine (pyrimido[4,5-d]pyrimidinyl).

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt thereof, or composition containing the substance or salt of the invention is suitable for administration to a subject or patient.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Pharmaceutically Acceptable Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds disclosed herein may also include other salts of such compounds which are not necessarily pharmaceutically acceptable salts, which may be useful as intermediates for one or more of the following: 1) preparing compounds disclosed herein; 2) purifying compounds disclosed herein; 3) separating enantiomers of compounds disclosed herein; or 4) separating diastereomers of compounds disclosed herein.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds disclosed herein may also include other solvates of such compounds which are not necessarily pharmaceutically acceptable solvates, which may be useful as intermediates for one or more of the following: 1) preparing compounds disclosed herein; 2) purifying compounds disclosed herein; 3) separating enantiomers of compounds disclosed herein;
or 4) separating diastereomers of compounds disclosed herein.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃—Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the Formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, one or more hydrogen atoms on certain metabolic sites on the compounds of the invention are deuterated.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR′->—NHR or —NHR′);
(iv) where the compound of the invention contains a secondary amino group, a primary derivative thereof (—NHR->—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or subcutaneous injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenous or subcutaneous doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: Formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### GIPR Antagonist Compounds

In one embodiment the present invention provides a GIPR antagonist compound of Formula I:

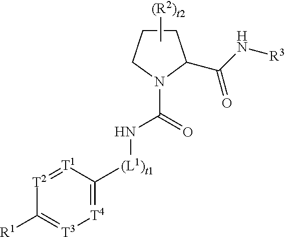

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

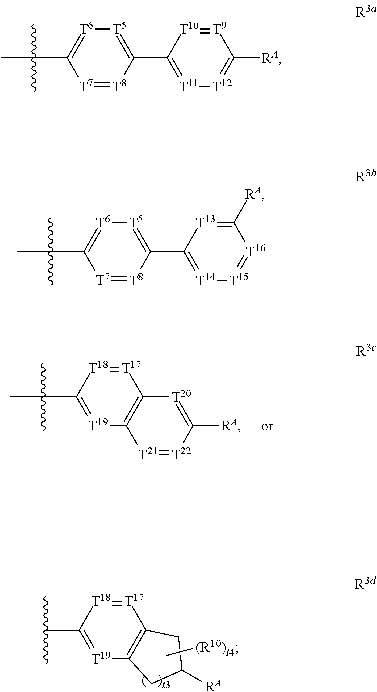

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹ and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4.

In one embodiment, the GIPR antagonist compound is a compound of Formula Ia:

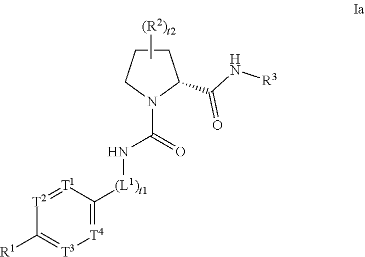

or a pharmaceutically acceptable salt thereof.

In one embodiment, the GIPR antagonist compound is a compound of Formula II:

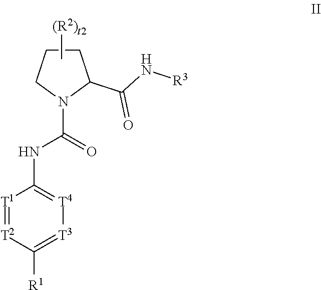

or a pharmaceutically acceptable salt thereof.

In one embodiment, the GIPR antagonist compound is a compound of Formula IIa:

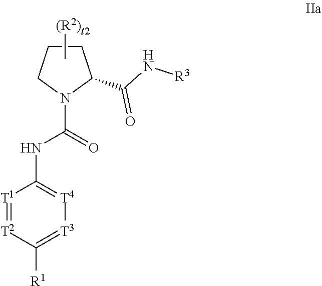

or a pharmaceutically acceptable salt thereof.

In one embodiment, the GIPR antagonist compound is a compound of Formula III:

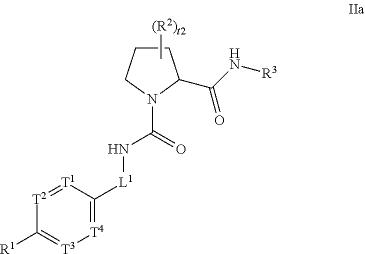

or a pharmaceutically acceptable salt thereof. In some embodiments, L¹ is CH₂.

In one embodiment, the GIPR antagonist compound is a compound of Formula IIIa:

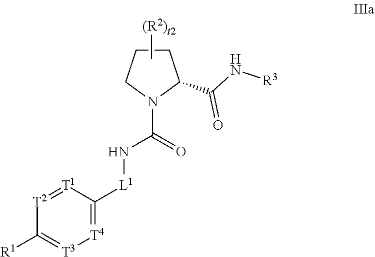

or a pharmaceutically acceptable salt thereof. In some embodiments, L¹ is CH₂.

In one embodiment, the GIPR antagonist compound is a compound of Formula IV:

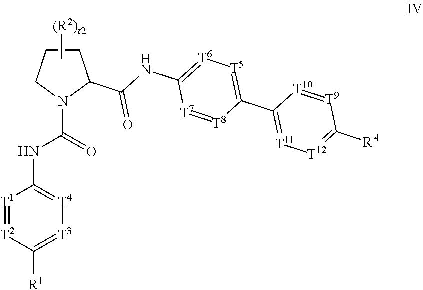

or a pharmaceutically acceptable salt thereof.

In one embodiment, the GIPR antagonist compound is a compound of Formula IV-1:

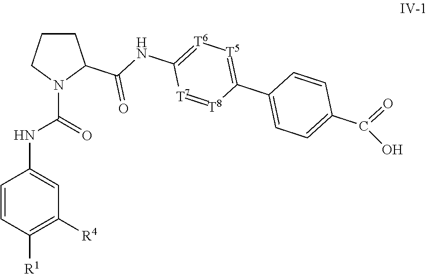

or a pharmaceutically acceptable salt thereof, wherein:
R¹ is propan-2-yl, prop-1-en-2-yl, trifluoromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵; and
each R⁵ is independently H, halogen, —CN, C₁₋₂ alkyl, C₁₋₂ haloalkyl, C₁₋₂ alkoxy, or C₁₋₂ haloalkoxy.

In some embodiments, the GIPR antagonist is a compound of Formula IV or IV-1, wherein R¹ is propan-2-yl, or trifluoromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. In some embodiments, the GIPR antagonist is a compound of Formula IV or IV-1, wherein R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is independently H, F, C₁₋₂ alkyl, C₁₋₂ haloalkyl, C₁₋₂ alkoxy, or C₁₋₂ haloalkoxy. In some embodiments, the GIPR antagonist is a compound of Formula IV or IV-1, wherein R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is H. In some embodiments, the GIPR antagonist is a compound of Formula IV or IV-1, wherein R¹ is propan-2-yl, or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy.

In one embodiment, the GIPR antagonist compound is a compound of Formula IV-2:

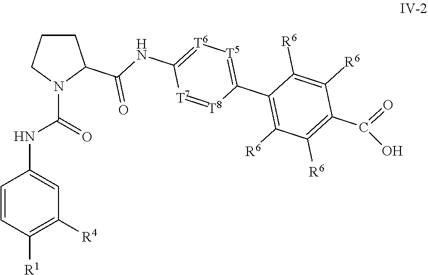

or a pharmaceutically acceptable salt thereof, wherein:
R¹ is propan-2-yl, prop-1-en-2-yl, trifluoromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵;
each R⁵ is independently H, halogen, —CN, C₁₋₂ alkyl, C₁₋₂ haloalkyl, C₁₋₂ alkoxy, or C₁₋₂ haloalkoxy; and
each R⁶ is independently H, halogen, —CN, C₁₋₂ alkyl, C₁₋₂ haloalkyl, C₁₋₂ alkoxy, or C₁₋₂ haloalkoxy, provided that at least one of the four R⁶ is other than H (e.g. one or two R⁶ are other than H).

In some embodiments, the GIPR antagonist is a compound of Formula IV-2, wherein R¹ is propan-2-yl or trifluoromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. In some embodiments, R¹ is propan-2-yl, or trifluoromethyl; R⁴ is H, F, or C₁₋₂ alkyl (e.g. methyl); each of T⁵, T⁶, T⁷, and T⁸ is independently CR⁵; each R⁵ is independently H, F, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl, or trifluoromethyl; R⁴ is H, F, or C₁₋₂ alkyl (e.g. methyl); each of T⁵, T⁶, T⁷, and T⁸ is independently CR⁵; each R⁵ is H; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that one of the four R⁶ is other than H and that the other three of the four R⁶ are H.

In one embodiment, the GIPR antagonist compound is a compound of Formula IVa:

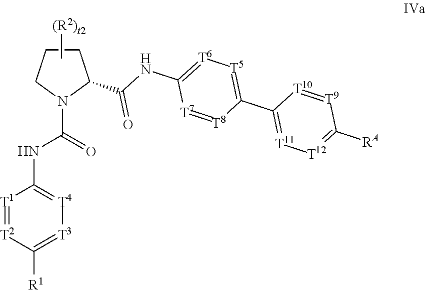

or a pharmaceutically acceptable salt thereof.

In one embodiment, the GIPR antagonist compound is a compound of Formula IVa-1:

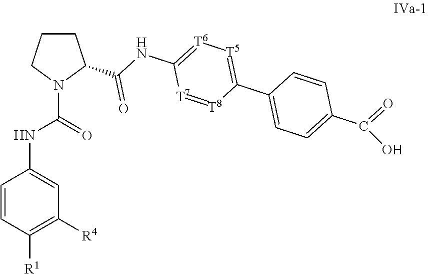

or a pharmaceutically acceptable salt thereof, wherein:
R¹ is propan-2-yl, prop-1-en-2-yl, trifluoromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵; and
each R⁵ is independently H, halogen, —CN, C₁₋₂ alkyl, C₁₋₂ haloalkyl, C₁₋₂ alkoxy, or C₁₋₂ haloalkoxy.

In some embodiments, the GIPR antagonist is a compound of Formula IV or IVa-1, wherein R¹ is propan-2-yl or trifluoromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. In some embodiments, R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is independently H, F, C₁₋₂ alkyl, C₁₋₂ haloalkyl, C₁₋₂ alkoxy, or C₁₋₂ haloalkoxy. In some embodiments, R¹ is propan-2-yl, or trifluoromethyl; R⁴ is H, F, or C₁₋₂ alkyl (e.g. methyl); each of T⁵, T⁶, T⁷, and T⁸ is independently CR⁵; and each R⁵ is H. In some embodiments, R¹ is propan-2-yl, or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy.

In one embodiment, the GIPR antagonist compound is a compound of Formula IVa-2:

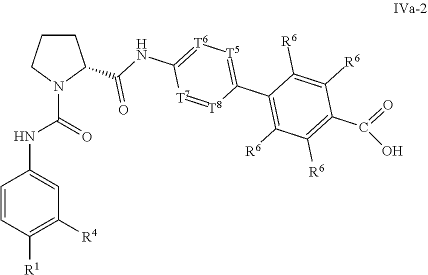

or a pharmaceutically acceptable salt thereof, wherein:
R¹ is propan-2-yl, prop-1-en-2-yl, trifluoromethyl, or cyclopropyl;
R⁴ is H, halo, or C₁₋₂ alkyl;
each of T⁵, T⁶, T⁷, and T⁸ is independently CR⁵, or one of T⁵, T⁶, T⁷, and T⁸ is N, and each of the other three of T⁵, T⁶, T⁷, and T⁸ is independently CR⁵;
each R⁵ is independently H, halogen, —CN, C₁₋₂ alkyl, C₁₋₂ haloalkyl, C₁₋₂ alkoxy, or C₁₋₂ haloalkoxy; and
each R⁶ is independently H, halogen, —CN, C₁₋₂ alkyl, C₁₋₂ haloalkyl, C₁₋₂ alkoxy, or C₁₋₂ haloalkoxy, provided that at least one of the four R⁶ is other than H (e.g. one or two R⁶ are other than H).

In some embodiments, the GIPR antagonist is a compound of Formula IV or IVa-2, wherein R¹ is propan-2-yl or trifluoromethyl; R⁴ is H, F, Cl, or C₁₋₂ alkyl; each of T⁵, T⁶, T⁷, and T⁸ is independently CR⁵; and each R⁵ is independently H, F, Cl, C₁₋₂ alkyl, C₁₋₂ haloalkyl, C₁₋₂ alkoxy, or C₁₋₂ haloalkoxy. In some embodiments, R¹ is propan-2-yl or trifluoromethyl; R⁴ is H, F, or C₁₋₂ alkyl (e.g. methyl); each of T⁵, T⁶, T⁷, and T⁸ is independently CR⁵; each R⁵ is independently H, F, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl or trifluoromethyl; R⁴ is H, F, or C₁₋₂ alkyl (e.g. methyl); each of T⁵, T⁶, T⁷, and T⁸ is independently CR⁵; each R⁵ is H; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that at least one of the four R⁶ is other than H. In some embodiments, R¹ is propan-2-yl or trifluoromethyl; R⁴ is H, F, Cl, or C₁₋₂ alkyl; one of T⁵, T⁶, T⁷, and T⁸ is N, and each of the other three of T⁵, T⁶, T⁷, and T⁸ is independently CR⁵; each R⁵ is independently H, F, Cl, C₁₋₂ alkyl, C₁₋₂ haloalkyl, C₁₋₂ alkoxy, or C₁₋₂ haloalkoxy; and each R⁶ is independently H, halogen, C₁₋₂ alkyl, C₁₋₂ haloalkyl, C₁₋₂ alkoxy, or C₁₋₂ haloalkoxy, provided that one of the four R⁶ is other than H and that the other three R⁶ are H.

In one embodiment, the GIPR antagonist compound is a compound of Formula V:

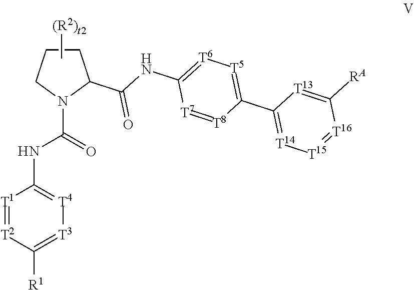

or a pharmaceutically acceptable salt thereof.

In one embodiment, the GIPR antagonist compound is a compound of Formula Va:

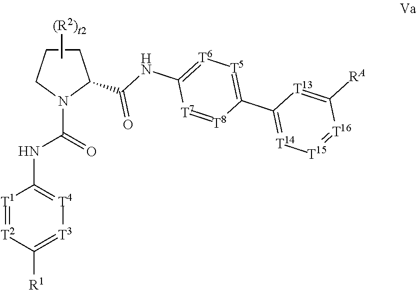

or a pharmaceutically acceptable salt thereof.

In one embodiment, the GIPR antagonist compound is a compound of Formula VI:

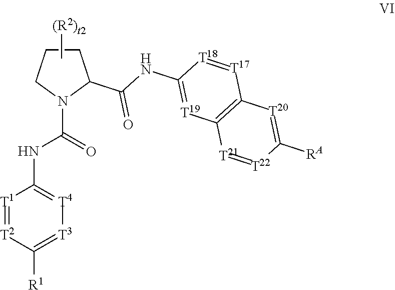

or a pharmaceutically acceptable salt thereof.

In one embodiment, the GIPR antagonist compound is a compound of Formula VIa:

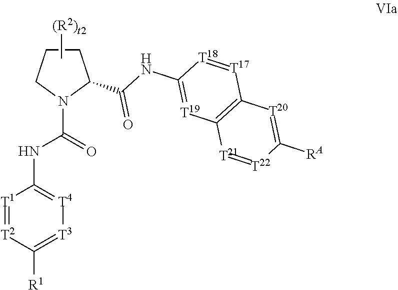

or a pharmaceutically acceptable salt thereof.

In one embodiment, the GIPR antagonist compound is a compound of Formula VII:

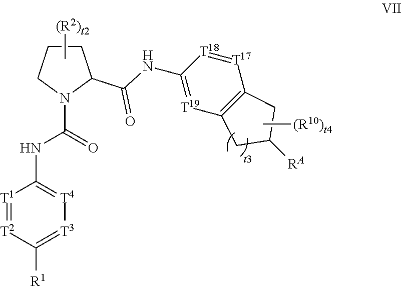

or a pharmaceutically acceptable salt thereof.

In one embodiment, the GIPR antagonist compound is a compound of Formula VIIa:

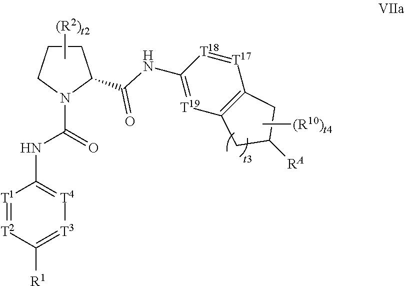

or a pharmaceutically acceptable salt thereof.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R¹ is halogen, —CN, C₁₋₈ alkyl, C₂₋₃ alkenyl, (C₃₋₆ cycloalkyl)-C₁₋₄ alkyl-, or C₃₋₆ cycloalkyl, wherein each of the C₁₋₈ alkyl, C₂₋₃ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy In some embodiments, R¹ is C₁₋₈ alkyl optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. In some embodiments, R¹ is C₂₋₆ alkyl optionally substituted with 1, 2, 3, 4, 5, or 6 substituents each independently selected from halogen, —OH, —CN, C₁₋₄ alkoxy, and C₁₋₄ haloalkoxy In some embodiments, R¹ is C₂₋₄ alkyl optionally substituted with 1, 2, 3, 4, 5, or 6 substituents each independently selected from halogen, —OH, —CN, C₁₋₄ alkoxy, and C₁₋₄ haloalkoxy. In some embodiments, R¹ is C₂₋₄ alkyl optionally substituted with 1, 2, 3, or 4 substituents each independently selected from halogen, —OH, —CN, C₁₋₄ alkoxy, and C₁₋₄ haloalkoxy. In some embodiments, R¹ is C₂₋₄ alkyl optionally substituted with 1, 2, 3, or 4 substituents each independently selected from halogen, —OH, C₁₋₄ alkoxy, and C₁₋₄ haloalkoxy.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R¹ is halogen, C₃₋₆ alkyl, C₃₋₆ alkenyl, (C₃₋₆ cycloalkyl)-C₁₋₄ alkyl-, or C₃₋₆ cycloalkyl, wherein each of the C₃₋₆ alkyl, C₃₋₆ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. In some embodiments, R¹ is cyclobutyl optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy. In some embodiments, R¹ is cyclobutyl optionally substituted with 1 or 2 substituents each independently selected from halogen, —OH, —CN, C₁₋₄ alkyl, C₁₋₄ haloalkyl, C₁₋₄ alkoxy, and C₁₋₄ haloalkoxy. In some embodiments, R¹ is C₃₋₄ alkyl optionally substituted with 1, 2, 3, 4, 5, or 6 substituents each independently selected from halogen, —OH, —CN, C₁₋₄ alkoxy, and C₁₋₄ haloalkoxy. In some embodiments, R¹ is C₃₋₄ alkyl optionally substituted with 1, 2, 3, or 4 substituents each independently selected from halogen, —OH, —CN, C₁₋₄ alkoxy, and C₁₋₄ haloalkoxy. In some embodiments, R¹ is C₃₋₄ alkyl optionally substituted with 1, 2, 3, or 4 substituents each independently selected from halogen, —OH, C₁₋₄ alkoxy, and C₁₋₄ haloalkoxy.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R¹ is cyclopropyl, cyclobutyl, R^{1a}, R^{1b}, or R^{1c},

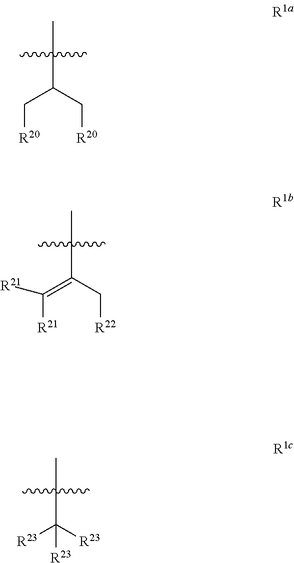

wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy. In some embodiments, R¹ is R^{1a}. In some embodiments, R¹ is R^{1a} and each R²⁰ is independently H, halogen, —OH, C₁₋₂ alkyl, or C₁₋₂ haloalkyl. In some embodiments, R¹ is R^{1a} and each R²⁰ is independently H, —OH, C₁₋₂ alkyl, or C₁₋₂ haloalkyl. In some embodiments, R¹ is R^{1a} and each R²⁰ is independently H, —OH, or C₁₋₂ alkyl. In some embodiments, R¹ is R^{1b}; each R²¹ is independently H or C₁₋₂ alkyl; and R²² is H, C₁₋₂ alkyl, or C₁₋₂ hydroxylalkyl. In some embodiments, R¹ is R^{1b}; each R²¹ is independently H or C₁₋₂ alkyl; and R²² is H or C₁₋₂ alkyl. In some embodiments, R¹ is R^{1c}. In some embodiments, R¹ is R^{1c}; and each R²³ is independently halogen, C₁₋₂ alkyl, C₁₋₂ hydroxylalkyl, or C₁₋₂ haloalkyl. In some embodiments, R¹ is R^{1c}; and each R²³ is independently C₁₋₂ alkyl, C₁₋₂ hydroxylalkyl, or C₁₋₂ haloalkyl. In some embodiments, R¹ is R^{1c}; and each R²³ is independently C₁₋₂ alkyl. In some embodiments, R¹ is R^{1c}; and each R²³ is methyl.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R¹ is propan-2-yl, prop-1-en-2-yl, trifluoromethyl, or cyclopropyl. In some embodiments, R¹ is propan-2-yl, prop-1-en-2-yl, or cyclopropyl. In some embodiments, R¹ is propan-2-yl, prop-1-en-2-yl, or trifluoromethyl. In some embodiments, R¹ is trifluoromethyl. In some embodiments, R¹ is propan-2-yl. In some embodiments, R¹ is prop-1-en-2-yl. In some embodiments, R¹ is cyclopropyl or cyclobutyl, each optionally substituted with 1 or 2 substituents, each of which is independently C₁₋₂ alkyl or C₁₋₂ haloalkyl. In some embodiments, R¹ is cyclopropyl or cyclobutyl, each optionally substituted with one C₁₋₂ alkyl or C₁₋₂ haloalkyl (e.g. CF₃). In some embodiments, R¹ is cyclopropyl optionally substituted with one C₁₋₂ alkyl or C₁₋₂ haloalkyl (e.g. CF₃). In some embodiments, R¹ is cyclopropyl.

In some embodiments, R¹ is cyclobutyl.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R¹ is C₁₋₄ haloalkyl or halo. In some embodiments, R¹ is C₁₋₄ haloalkyl, for example, R¹ is C₁₋₂ haloalkyl. In some embodiments, R¹ is C₁₋₂ haloalkyl, for example, R¹ is C₁₋₂ fluoroalkyl. In some embodiments, R¹ is trifluoromethyl.

In some embodiments, R¹ is halo, for example, Cl.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T¹, T², T³, and T⁴ is independently CR⁴. In some embodiments, each of T¹, T², and T⁴ is CH; and T³ is CR⁴. In some embodiments, each of T¹, T², and T⁴ is CH; T³ is CR⁴; and R⁴ is H, halo, C₁₋₂ alkyl, or C₁₋₂ haloalkyl. In some embodiments, each of T¹, T², and T⁴ is CH; T³ is CR⁴; and R⁴ is H, F, or methyl.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein one of T¹, T², T³, and T⁴ is N, and the other three are each independently CR⁴. In some embodiments, T¹ is N, and each of T², T³, and T⁴ is independently CR⁴. In some embodiments, T² is N, and each of T¹, T³, and T⁴ is independently CR⁴. In some embodiments, T¹, T², T³, and T⁴ are N, and the other two are each independently CR⁴.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁴ is independently H, halo, C₁₋₂ alkyl, or C₁₋₂ haloalkyl. In some embodiments, each R⁴ is independently H, halo, or C₁₋₂ alkyl. In some embodiments, each R⁴ is independently H, F, or methyl. In some embodiments, each R⁴ is independently H or F. In some embodiments, each R⁴ is independently H or F. In some embodiments, each R⁴ is independently H or C₁₋₂ alkyl. In some embodiments, each R⁴ is independently H or methyl. In some embodiments, each R⁴ is H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-; and t2 is 0 or 1. In some embodiments, each R² is independently halogen, —OH, C₁₋₂ alkyl, C₁₋₂ hydroxylalkyl, C₁₋₂ haloalkyl, C₁₋₂ alkoxy, C₁₋₂ haloalkoxy, or C₃₋₄ cycloalkyl; and t2 is 0 or 1. In some embodiments, R² is —OH, C₁₋₂ alkyl, C₁₋₂ hydroxylalkyl, C₁₋₂ haloalkyl, C₁₋₂ alkoxy, or C₁₋₂ haloalkoxy; and t2 is 0 or 1. In some embodiments, R² is —OH, C₁₋₂ alkyl, or C₁₋₂ alkoxy; and t2 is 0 or 1.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t2 is 0. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t2 is 2 or 3; and two R², which are attached to two adjacent ring carbon atoms of the proline ring in Formula I, together with the two ring carbon atoms to which they are attached, form C₃₋₆ cycloalkyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. In some embodiments, t2 is 2; and two R², which are attached to two adjacent ring carbon atoms of the proline ring in Formula I, together with the two ring carbon atoms to which they are attached, form C₃₋₆ cycloalkyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. In some embodiments, t2 is 2; and two R², which are attached to two adjacent ring carbon atoms of the proline ring in Formula I, together with the two ring carbon atoms to which they are attached, form cyclopropyl that is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy. In some embodiments, t2 is 2; and two R², which are attached to two adjacent ring carbon atoms of the proline ring in Formula I, together with the two ring carbon atoms to which they are attached, form cyclopropyl fused to the proline ring, and the resulting fused bicyclic ring is a 3-azabicyclo[3.1.0]hexane ring.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T⁵, T⁶, T⁷, and T⁸ is independently CR⁵. In some embodiments, each of T⁵, T⁶, T⁷, and T⁸ is CH. In some embodiments, three of T⁵, T⁶, T⁷, and T⁸ are CH; one of T⁵, T⁶, T⁷, and T⁸ is CR⁵; and R⁵ is halogen, —CN, C₁₋₂ alkyl, C₁₋₂ haloalkyl, C₁₋₂ alkoxy, or C₁₋₂ haloalkoxy. In some embodiments, three of T⁵, T⁶, T⁷, and T⁸ are CH; one of T⁵, T⁶, T⁷, and T⁸ is CR⁵; and R⁵ is F, Cl, C₁₋₂ alkyl, C₁₋₂ haloalkyl, C₁₋₂ alkoxy, or C₁₋₂ haloalkoxy. In some embodiments, three of T⁵, T⁶, T⁷, and T⁸ are CH; one of T⁵, T⁶, T⁷, and T⁸ is CR⁵; and R⁵ is F, methyl, or methoxy. In some embodiments, at least one of the four R⁵ in T⁵, T⁶, T⁷, and T⁸ is other than H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein one of T⁵, T⁶, T⁷, and T⁸ is N and the other three are each independently CR⁵. In some embodiments, each of the three R⁵ is H. In some embodiments, one of the three R⁵ is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁵ is F, Cl, C₁₋₂ alkyl, C₁₋₂ haloalkyl, C₁₋₂ alkoxy, or C₁₋₂ haloalkoxy; and the other two R⁵ are H. In some embodiments, one of the three R⁵ is F, methyl, or methoxy; and the other two R⁵ are H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T⁵ is N and each of T⁶, T⁷, and T⁸ is independently CR⁵. In some embodiments, each of the three R⁵ is H. In some embodiments, one of the three R⁵ (e.g. the R⁵ in T⁸) is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁵ (e.g. the R⁵ in T⁸) is F, Cl, C₁₋₂ alkyl, C₁₋₂ haloalkyl, C₁₋₂ alkoxy, or C₁₋₂ haloalkoxy; and the other two R⁵ are H. In some embodiments, one of the three R⁵ (e.g. the R⁵ in T⁸) is F, methyl, or methoxy; and the other two R⁵ are H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T⁶ is N and each of T⁵, T⁷, and T⁸ is independently CR⁵. In some embodiments, each of the three R⁵ is H. In some embodiments, one of the three R⁵ is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁵ is F, Cl, C₁₋₂ alkyl, C₁₋₂ haloalkyl, C₁₋₂ alkoxy, or C₁₋₂ haloalkoxy; and the other two R⁵ are H. In some embodiments, one of the three R⁵ is F, methyl, or methoxy; and the other two R⁵ are H. In some embodiments, two of T⁵, T⁶, T⁷, and T⁸ are N and the other two are each independently CR⁵.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁵ is independently H, halo, C₁₋₂ alkyl, or C₁₋₂ haloalkyl. In some embodiments, each R⁵ is independently H, halo, or C₁₋₂ alkyl. In some embodiments, each R⁵ is independently H or halo. In some embodiments, each R⁵ is H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T⁹, T¹⁰, T¹¹, and T¹² is independently CR⁶. In some embodiments, each of T⁹, T¹⁰, T¹¹, and T¹² is CH. In some embodiments, three of T⁹, T¹⁰, T¹¹, and T¹² are CH; one of T⁹, T¹⁰, T¹¹, and T¹² (e.g. T⁹) is CR⁶; and R⁶ is halogen, —CN, C₁₋₂ alkyl, C₁₋₂ haloalkyl, C₁₋₂ alkoxy, or C₁₋₂ haloalkoxy. In some embodiments, three of T⁹, T¹⁰, T¹¹, and T¹² are CH; one of T⁹, T¹⁰, T¹¹, and T¹² (e.g. T⁹) is CR⁶; and R⁶ is F, Cl, C₁₋₂ alkyl, C₁₋₂ haloalkyl, C₁₋₂ alkoxy, or C₁₋₂ haloalkoxy. In some embodiments, three of T⁹, T¹⁰, T¹¹, and T¹² are CH; one of T⁹, T¹⁰, T¹¹, and T¹² (e.g. T⁹) is CR⁶; and R⁶ is F, methyl, or methoxy. In some embodiments, at least one of the four R⁶ in T⁹, T¹⁰, T¹¹, and T¹² is other than H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein one of T⁹, T¹⁰, T¹¹, and T¹² is N and the other three are each independently CR⁶. In some embodiments, each of the three R⁶ is H. In some embodiments, one of the three R⁶ is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁶ is halogen, C₁₋₂ alkyl, or C₁₋₂ haloalkyl; and the other two R⁶ are H. In some embodiments, one of the three R⁶ is F or methyl; and the other two R⁵ are H. In some embodiments, one of the three R⁶ is methyl; and the other two R⁵ are H. In some embodiments, one of the three R⁶ is halogen; and the other two R⁵ are H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T⁹ is N and each of T¹⁰, T¹¹, and T¹² is independently CR⁶. In some embodiments, each of the three R⁶ is H. In some embodiments, one of the three R⁶ (e.g. the R⁶ in T¹⁰ or T¹²) is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁶ (e.g. the R⁶ in T¹⁰) is C₁₋₂ alkyl or C₁₋₂ haloalkyl; and the other two R⁶ are H. In some embodiments, one of the three R⁶ (e.g. the R⁶ in T¹⁰) is methyl; and the other two R⁵ are H. In some embodiments, one of the three R⁶ (e.g. the R⁶ in T¹²) is halogen, C₁₋₂ alkyl, C₁₋₂ haloalkyl, C₁₋₂ alkoxy, or C₁₋₂ haloalkoxy; and the other two R⁵ are H. In some embodiments, one of the three R⁶ (e.g. the R⁶ in T¹²) is halogen or C₁₋₂ alkyl; and the other two R⁵ are H. In some embodiments, one of the three R⁶ (e.g. the R⁶ in T¹²) is F; and the other two R⁵ are H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T¹⁰ is N and each of T⁹, T¹¹, and T¹² is independently CR⁶. In some embodiments, each of the three R⁶ is H. In some embodiments, one of the three R⁶ (e.g. the R⁶ in T⁹ or T¹¹) is halogen, —CN, C₁₋₂ alkyl, C₁₋₂ haloalkyl, C₁₋₂ alkoxy, or C₁₋₂ haloalkoxy; and the other two R⁵ are H. In some embodiments, one of the three R⁶ (e.g. the R⁶ in T⁹) is C₁₋₂ alkyl or C₁₋₂ haloalkyl; and the other two R⁶ are H. In some embodiments, one of the three R⁶ (e.g. the R⁶ in T⁹ or T¹¹) is methyl; and the other two R⁵ are H. In some embodiments, one of the three R⁶ (e.g. the R⁶ in T¹¹) is halogen, C₁₋₂ alkyl, C₁₋₂ haloalkyl, C₁₋₂ alkoxy, or C₁₋₂ haloalkoxy; and the other two R⁵ are H. In some embodiments, one of the three R⁶ (e.g. the R⁶ in T¹¹) is halogen or C₁₋₂ alkyl; and the other two R⁵ are H. In some embodiments, one of the three R⁶ (e.g. the R⁶ in T¹¹) is F; and the other two R⁵ are H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T⁹ is N, T¹⁰ is CH or C(CH₃), T¹¹ is CH, and T¹² is CH. In some embodiments, T⁹ is N, T¹⁰ is C(CH₃), T¹¹ is CH, and T¹² is CH. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein two of T⁹, T¹⁰, T¹¹, and T¹² are N and the other two are each independently CR⁶. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T¹⁰ and T¹¹ is N and each of T⁹ and T¹² is independently CR⁶.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁶ is independently H, halo, C₁₋₂ alkyl, or C₁₋₂ haloalkyl. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁶ is independently H, halo, or C₁₋₂ alkyl. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁶ is independently H or halo. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁶ is independently H or C₁₋₂ alkyl.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T¹³, T¹⁴, T¹⁵, and T¹⁶ is independently CR⁷. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein one of T¹³, T¹⁴, T¹⁵, and T¹⁶ is N and the other three are each independently CR⁷. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein T¹³ is N and each of T¹⁴, T¹⁵, and T¹⁶ is independently CR⁷. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein two of T¹³, T¹⁴, T¹⁵, and T¹⁶ are N and the other two are each independently CR⁷.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each R⁷ is independently H, halo, C₁₋₂ alkyl, or C₁₋₂ haloalkyl. In some embodiments, each R⁷ is independently H, halo, or C₁₋₂ alkyl. In some embodiments, each R⁷ is independently H or halo. In some embodiments, each R⁷ is independently H or C₁₋₂ alkyl. In some embodiments, each R⁷ is H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T¹⁷, T¹⁸, and T¹⁹ is independently CR⁸. In some embodiments, one of T¹⁷, T¹⁸, and T¹⁹ is N, and the other two are independently CR⁸. In some embodiments, each R⁸ is independently H, halo, C₁₋₂ alkyl, or C₁₋₂ haloalkyl. In some embodiments, each R⁸ is independently H, halo, or C₁₋₂ alkyl. In some embodiments, each R⁸ is independently H or C₁₋₂ alkyl. In some embodiments, each R⁸ is independently H or halo. In some embodiments, each R⁸ is H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein each of T²⁰, T²¹, and T²² is independently CR⁹. In some embodiments, one of T²⁰, T²¹, and T¹¹ is N, and the other two are each independently CR⁹. In some embodiments, T²⁰ is N, and each of T²¹ and T²² is independently CR⁹. In some embodiments, each R⁹ is independently H, halo, C₁₋₂ alkyl, or C₁₋₂ haloalkyl. In some embodiments, each R⁹ is independently H, halo, or C₁₋₂ alkyl. In some embodiments, each R⁹ is independently H or halo. In some embodiments, each R⁹ is independently H or C₁₋₂ alkyl. In some embodiments, each R⁹ is H.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t3 is 1. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t3 is 2 (e.g. wherein the compound has the structure of Formula VII or Formula VIIa and t3 is 2, or a pharmaceutically acceptable salt thereof). In some embodiments, each of T¹⁷ and T¹⁸ is independently CR⁸; and T¹⁹ is N. In some embodiments, each of T¹⁷ and T¹⁸ is CH; and T¹⁹ is N. In some embodiments, each of T¹⁷ and T¹⁸ is independently CR⁸; T¹⁹ is N, and R^{A} is C(═O)OH. In some embodiments, each of T¹⁷ and T¹⁸ is independently CH; T¹⁹ is N, and R^{A} is C(═O)OH.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t4 is 0, 1, or 2; and each R¹⁰ is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein t4 is 0 or 1; and each R¹⁰ is halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R^{A} is —C(═O)—OH. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R^{A} is —C(═O)—NH₂. In some embodiments, the GIPR antagonist is a compound of Formula I, Ia, II, IIa, III, IIIa, IV, IV-1, IV-2, IVa, IVa-1, IVa-2, V, Va, VI, Via, VII, or VIIa, wherein R^{A} is —OH.

The GIPR antagonist compounds of the disclosure are compounds comprising a molecular weight of from about 400 Da to about 600 Da, from about 400 Da to about 500 Da, from about 500 Da to about 600 Da, from about 450 Da to about 550 Da, from about 400 Da to about 450 Da, from about 450 Da to about 500 Da, from about 500 Da to about 550 Da, from about 550 Da to about 600 Da. In some embodiments, the GIPR antagonist compounds of the disclosure comprise a molecular weight of from about 450 Da to about 500 Da. In some embodiments, the GIPR antagonist compounds of the disclosure comprise a molecular weight of from about 500 Da to about 550 Da.

TABLE 1 shows GIPR antagonist compounds of the disclosure that can be used in combination with the GLP1R agonist compounds disclosed herein.

TABLE 1
GIPR Antagonist Compounds of the disclosure

| GIPR | | |
|---|---|---|
| Ant | Structure | IUPAC Name |
| 1A | 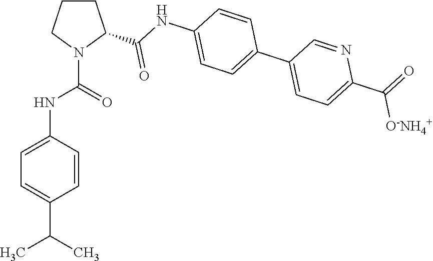 | Ammonium 5-{4-[(1-{[4-(propan-2- yl)phenyl]carbamoyl}-D- prolyl)amino]phenyl}pyridine-2-carboxylate |
| 2A | 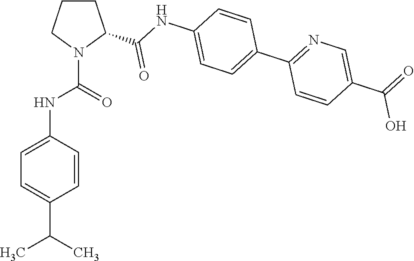 [SMILES: CC(C)c1ccc(NC(=O)N2CCC[C@@H]2C(=O)Nc2ccc(-c3ccc(C(=O)O)cn3)cc2)cc1] | 6-{4-[(1-{[4-(Propan-2- yl)phenyl]carbamoyl}-D- prolyl)amino]phenyl}pyridine-3-carboxylic acid |
| 3A | 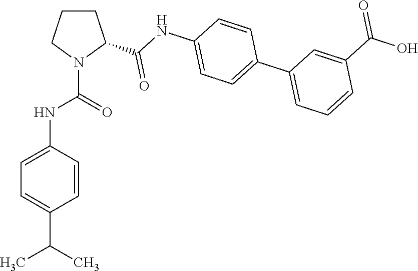 [SMILES: CC(C)c1ccc(NC(=O)N2CCC[C@@H]2C(=O)Nc2ccc(-c3cccc(C(=O)O)c3)cc2)cc1] | 4′-[(1-{[4-(Propan-2-yl)phenyl]carbamoyl}- D-prolyl)amino][1,1′-biphenyl]-3-carboxylic acid |
| 4A | 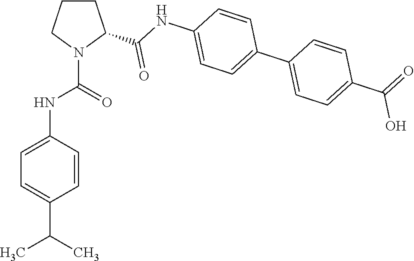 [SMILES: CC(C)c1ccc(NC(=O)N2CCC[C@@H]2C(=O)Nc2ccc(-c3ccc(C(=O)O)cc3)cc2)cc1] | 4′-[(1-{[4-(Propan-2-yl)phenyl]carbamoyl}- D-prolyl)amino][1,1′-biphenyl]-4-carboxylic acid |
| 5A | 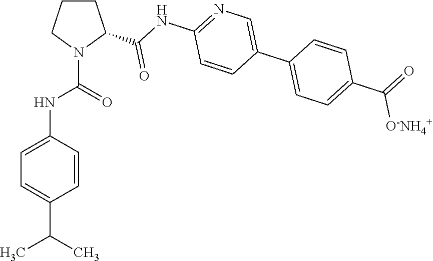 | Ammonium 4-{6-[(1-{[4-(propan-2- yl)phenyl]carbamoyl}-D- prolyl)amino]pyridin-3-yl}benzoate |
| 6A | 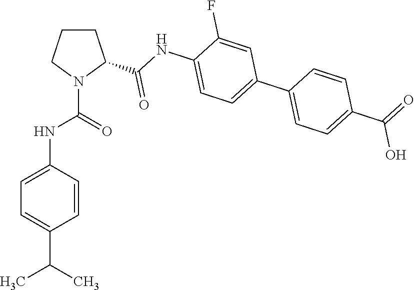 [SMILES: CC(C)c1ccc(NC(=O)N2CCC[C@@H]2C(=O)Nc2ccc(-c3ccc(C(=O)O)cc3)cc2F)cc1] | 3′-Fluoro-4′-[(1-{[4-(propan-2- yl)phenyl]carbamoyl}-D-prolyl)amino][1,1′- biphenyl]-4-carboxylic acid |
| 7A | 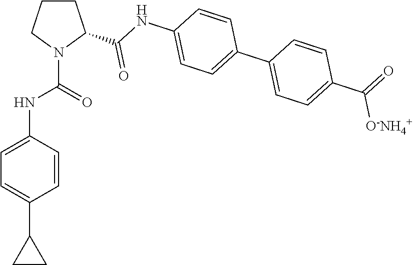 | Ammonium 4′-({1-[(4- cyclopropylphenyl)carbamoyl]-D- prolyl}amino)[1,1′-biphenyl]-4-carboxylate |
| 8A | 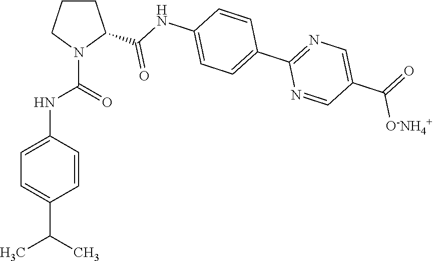 | ammonium 2-{4-[(1-{[4-(propan-2- yl)phenyl]carbamoyl}-D- prolyl)amino]phenyl}pyrimidine-5- carboxylate |
| 9A | 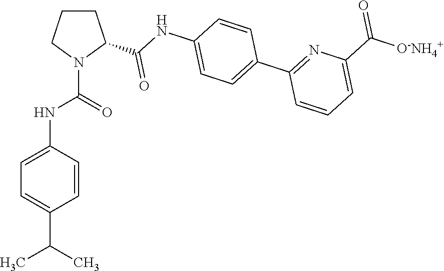 | ammonium 6-{4-[(1-{[4-(propan-2- yl)phenyl]carbamoyl}-D- prolyl)amino]phenyl}pyridine-2-carboxylate |
| 10A | 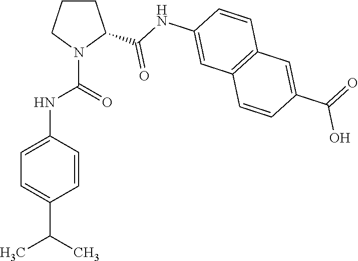 [SMILES: CC(C)c1ccc(NC(=O)N2CCC[C@@H]2C(=O)Nc2ccc3cc(C(=O)O)ccc3c2)cc1] | 6-[(1-{[4-(propan-2-yl)phenyl]carbamoyl}- D-prolyl)amino]naphthalene-2-carboxylic acid |
| 11A | 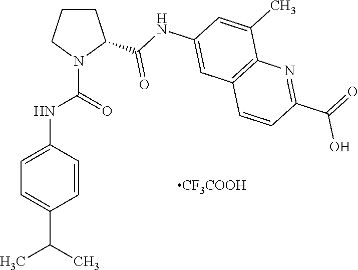 [SMILES: Cc1cc(NC(=O)[C@H]2CCCN2C(=O)Nc2ccc(C(C)C)cc2)cc2ccc(C(=O)O)nc12 ; "·CF3COOH"] | 8-methyl-6-[(1-{[4-(propan-2- yl)phenyl]carbamoyl}-D- prolyl)amino]quinoline-2-carboxylic acid, trifluoroacetate salt |
| 12A | 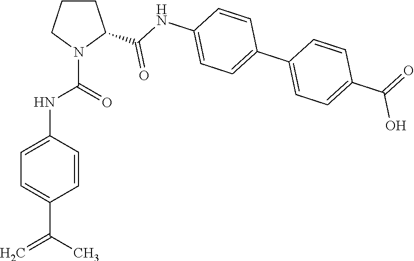 [SMILES: C=C(C)c1ccc(NC(=O)N2CCC[C@@H]2C(=O)Nc2ccc(-c3ccc(C(=O)O)cc3)cc2)cc1] | 4′-[(1-{[4-(prop-1-en-2- yl)phenyl]carbamoyl}-D-prolyl)amino][1,1′- biphenyl]-4-carboxylic acid |
| 13A | 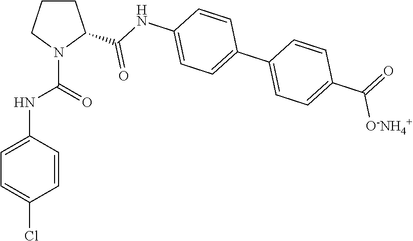 | ammonium 4′-({1-[(4- chlorophenyl)carbamoyl]-D- prolyl}amino)[1,1′-biphenyl]-4-carboxylate |
| 14A | 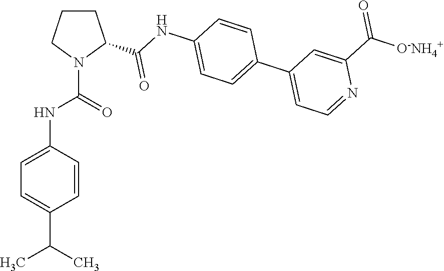 | ammonium 4-{4-[(1-{[4-(propan-2- yl)phenyl]carbamoyl}-D- prolyl)amino]phenyl}pyridine-2-carboxylate |
| 15A | 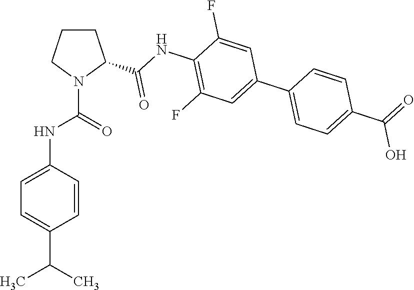 [SMILES: CC(C)c1ccc(NC(=O)N2CCC[C@@H]2C(=O)Nc2c(F)cc(-c3ccc(C(=O)O)cc3)cc2F)cc1] | 3′,5′-difluoro-4′-[(1-{[4-(propan-2- yl)phenyl]carbamoyl}-D-prolyl)amino][1,1′- biphenyl]-4-carboxylic acid |
| 16A | 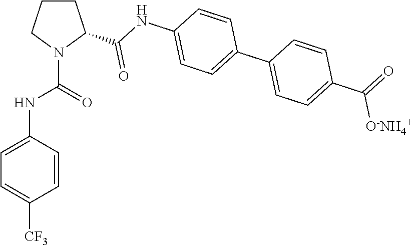 | Ammonium 4′-[(1-{[4- (trifluoromethyl)phenyl]carbamoyl}-D- prolyl)amino][1,1′-biphenyl]-4-carboxylate |
| 17A | 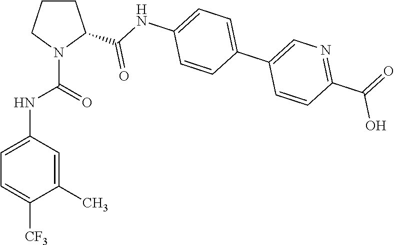 [SMILES: Cc1cc(NC(=O)N2CCC[C@@H]2C(=O)Nc2ccc(-c3ccc(C(=O)O)nc3)cc2)ccc1C(F)(F)F] | 5-{4-[(1-{[3-Methyl-4- (trifluoromethyl)phenyl]carbamoyl}-D- prolyl)amino]phenyl}pyridine-2-carboxylic acid |
| 18A | 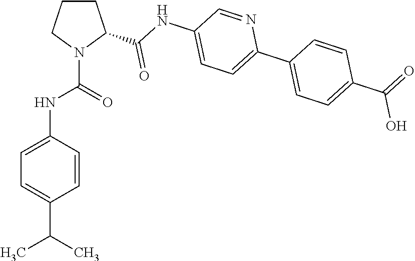 [SMILES: CC(C)c1ccc(NC(=O)N2CCC[C@@H]2C(=O)Nc2ccc(-c3ccc(C(=O)O)cc3)nc2)cc1] | 4-{5-[(1-{[4-(Propan-2- yl)phenyl]carbamoyl}-D- prolyl)amino]pyridin-2-yl}benzoic acid |
| 19A | 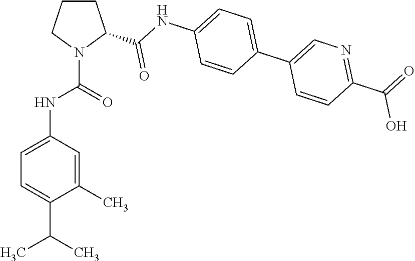 [SMILES: Cc1cc(NC(=O)N2CCC[C@@H]2C(=O)Nc2ccc(-c3ccc(C(=O)O)nc3)cc2)ccc1C(C)C] | 5-{4-[(1-{[3-Methyl-4-(propan-2- yl)phenyl]carbamoyl}-D- prolyl)amino]phenyl}pyridine-2-carboxylic acid |
| 20A | 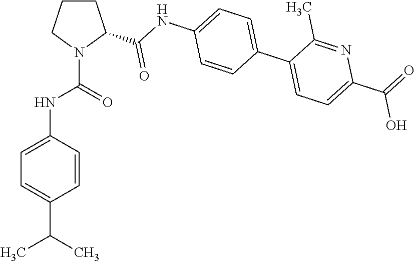 [SMILES: Cc1nc(C(=O)O)ccc1-c1ccc(NC(=O)[C@H]2CCCN2C(=O)Nc2ccc(C(C)C)cc2)cc1] | 6-Methyl-5-{4-[(1-{[4-(propan-2- yl)phenyl]carbamoyl}-D- prolyl)amino]phenyl}pyridine-2-carboxylic acid |
| 21A | 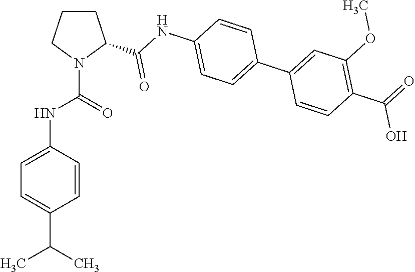 [SMILES: COc1cc(-c2ccc(NC(=O)[C@H]3CCCN3C(=O)Nc3ccc(C(C)C)cc3)cc2)ccc1C(=O)O] | 3-Methoxy-4′-[(1-{[4-(propan-2- yl)phenyl]carbamoyl}-D-prolyl)amino][1,1′- biphenyl]-4-carboxylic acid |
| 22A | 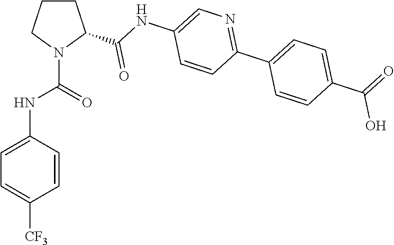 [SMILES: O=C(O)c1ccc(-c2ccc(NC(=O)[C@H]3CCCN3C(=O)Nc3ccc(C(F)(F)F)cc3)cn2)cc1] | 4-{5-[(1-{[4- (Trifluoromethyl)phenyl]carbamoyl}-D- prolyl)amino]pyridin-2-yl}benzoic acid |
| 23A | 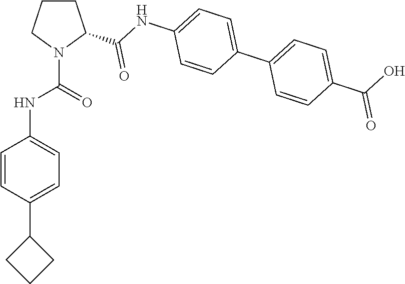 [SMILES: O=C(O)c1ccc(-c2ccc(NC(=O)[C@H]3CCCN3C(=O)Nc3ccc(C4CCC4)cc3)cc2)cc1] | 4′-({1-[(4-Cyclobutylphenyl)carbamoyl]-D- prolyl}amino)[1,1′-biphenyl]-4-carboxylic acid |
| 24A | 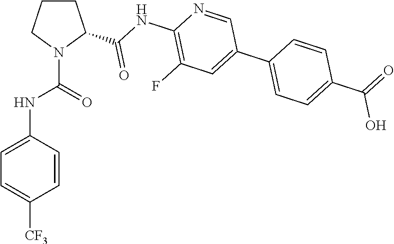 [SMILES: O=C(O)c1ccc(-c2cnc(NC(=O)[C@H]3CCCN3C(=O)Nc3ccc(C(F)(F)F)cc3)c(F)c2)cc1] | 4-{5-Fluoro-6-[(1-{[4- (trifluoromethyl)phenyl]carbamoyl}-D- prolyl)amino]pyridin-3-yl}benzoic acid |
| 25A | 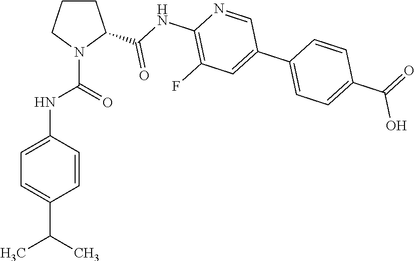 [SMILES: CC(C)c1ccc(NC(=O)N2CCC[C@@H]2C(=O)Nc2ncc(-c3ccc(C(=O)O)cc3)cc2F)cc1] | 4-{5-Fluoro-6-[(1-{[4-(propan-2- yl)phenyl]carbamoyl}-D- prolyl)amino]pyridin-3-yl}benzoic acid |
| 26A | 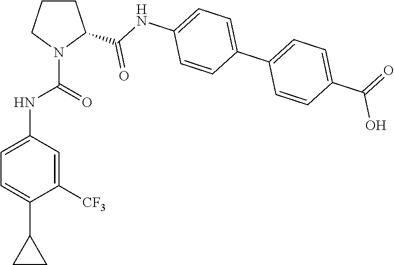 [SMILES: O=C(O)c1ccc(-c2ccc(NC(=O)[C@H]3CCCN3C(=O)Nc3ccc(C4CC4)c(C(F)(F)F)c3)cc2)cc1] | 4′-[(1-{[4-Cyclopropyl-3- (trifluoromethyl)phenyl]carbamoyl}-D- prolyl)amino][1,1′-biphenyl]-4-carboxylic acid |
| 27A | 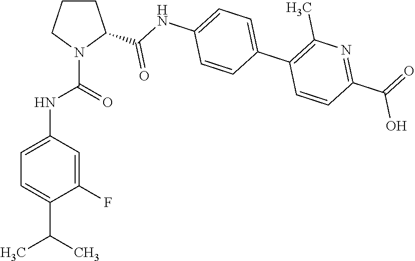 [SMILES: Cc1nc(C(=O)O)ccc1-c1ccc(NC(=O)[C@H]2CCCN2C(=O)Nc2ccc(C(C)C)c(F)c2)cc1] | 5-{4-[(1-{[3-Fluoro-4-(propan-2- yl)phenyl]carbamoyl}-D- prolyl)amino]phenyl}-6-methylpyridine-2- carboxylic acid |
| 28A | 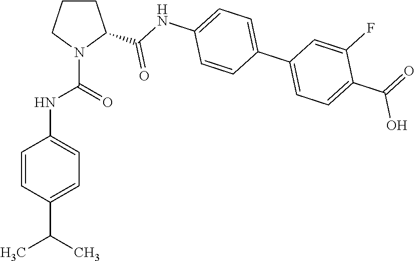 [SMILES: CC(C)c1ccc(NC(=O)N2CCC[C@@H]2C(=O)Nc2ccc(-c3ccc(C(=O)O)c(F)c3)cc2)cc1] | 3-Fluoro-4′-[(1-{[4-(propan-2- yl)phenyl]carbamoyl}-D-prolyl)amino][1,1′- biphenyl]-4-carboxylic acid |
| 29A | 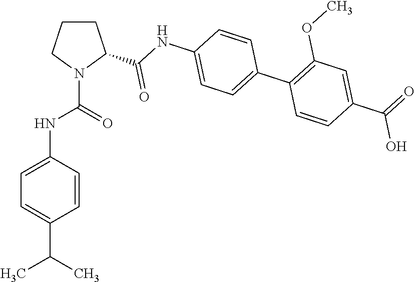 [SMILES: COc1cc(C(=O)O)ccc1-c1ccc(NC(=O)[C@H]2CCCN2C(=O)Nc2ccc(C(C)C)cc2)cc1] | 2-Methoxy-4′-[(1-{[4-(propan-2- yl)phenyl]carbamoyl}-D-prolyl)amino][1,1′- biphenyl]-4-carboxylic acid |
| 30A | 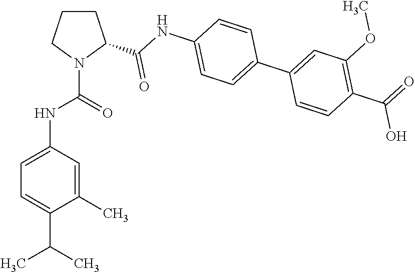 [SMILES: COc1cc(-c2ccc(NC(=O)[C@H]3CCCN3C(=O)Nc3ccc(C(C)C)c(C)c3)cc2)ccc1C(=O)O] | 3-Methoxy-4′-[(1-{[3-methyl-4-(propan-2- yl)phenyl]carbamoyl}-D-prolyl)amino][1,1′- biphenyl]-4-carboxylic acid |
| 31A | 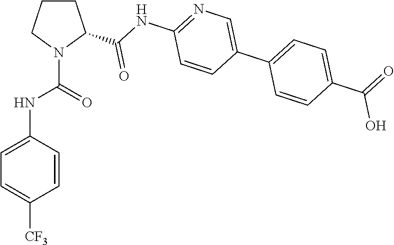 [SMILES: O=C(O)c1ccc(-c2ccc(NC(=O)[C@H]3CCCN3C(=O)Nc3ccc(C(F)(F)F)cc3)nc2)cc1] | 4-{6-[(1-{[4- (Trifluoromethyl)phenyl]carbamoyl}-D- prolyl)amino]pyridin-3-yl}benzoic acid |
| 32A | 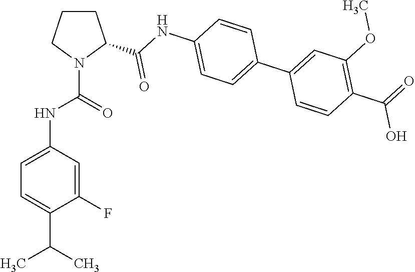 [SMILES: COc1cc(-c2ccc(NC(=O)[C@H]3CCCN3C(=O)Nc3ccc(C(C)C)c(F)c3)cc2)ccc1C(=O)O] | 4′-[(1-{[3-Fluoro-4-(propan-2- yl)phenyl]carbamoyl}-D-prolyl)amino]-3- methoxy[1,1′-biphenyl]-4-carboxylic acid |
| 33A | 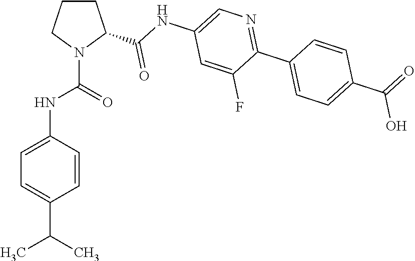 [SMILES: CC(C)c1ccc(NC(=O)N2CCC[C@@H]2C(=O)Nc2cnc(-c3ccc(C(=O)O)cc3)c(F)c2)cc1] | 4-{3-Fluoro-5-[(1-{[4-(propan-2- yl)phenyl]carbamoyl}-D- prolyl)amino]pyridin-2-yl}benzoic acid |
| 34A | 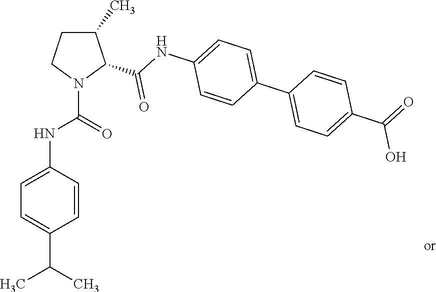 [SMILES: CC(C)c1ccc(NC(=O)N2CC[C@H](C)[C@@H]2C(=O)Nc2ccc(-c3ccc(C(=O)O)cc3)cc2)cc1] | 4′-{[(3S)-3-Methyl-1-{[4-(propan-2- yl)phenyl]carbamoyl}-D-prolyl]amino}[1,1′- biphenyl]-4-carboxylic acid or 4′-{[(3R)-3- Methyl-1-{[4-(propan-2- yl)phenyl]carbamoyl}-L-prolyl]amino}[1,1′- biphenyl]-4-carboxylic acid |
| | 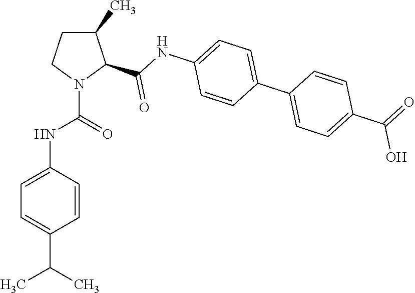 [SMILES: CC(C)c1ccc(NC(=O)N2CC[C@@H](C)[C@H]2C(=O)Nc2ccc(-c3ccc(C(=O)O)cc3)cc2)cc1] | |
| 35A | 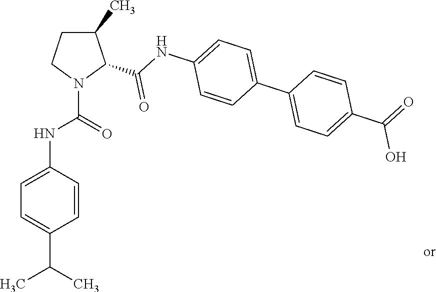 [SMILES: CC(C)c1ccc(NC(=O)N2CC[C@@H](C)[C@@H]2C(=O)Nc2ccc(-c3ccc(C(=O)O)cc3)cc2)cc1] | 4′-{[(3R)-3-Methyl-1-{[4-(propan-2- yl)phenyl]carbamoyl}-D-prolyl]amino}[1,1′- biphenyl]-4-carboxylic acid or 4′-{[(3S)-3- Methyl-1-{[4-(propan-2- yl)phenyl]carbamoyl}-L-prolyl]amino}[1,1′- biphenyl]-4-carboxylic acid |
| | 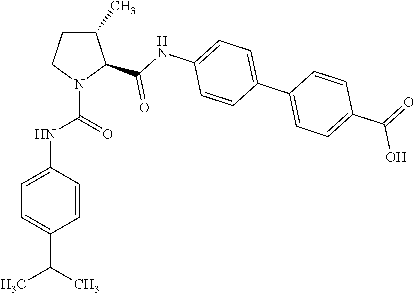 [SMILES: CC(C)c1ccc(NC(=O)N2CC[C@H](C)[C@H]2C(=O)Nc2ccc(-c3ccc(C(=O)O)cc3)cc2)cc1] | |
| 36A | 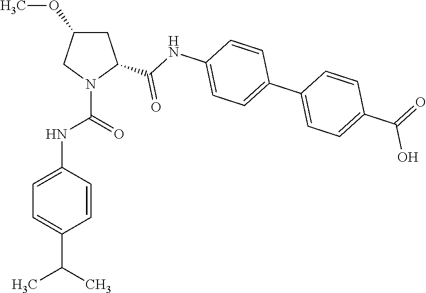 [SMILES: CO[C@@H]1C[C@H](C(=O)Nc2ccc(-c3ccc(C(=O)O)cc3)cc2)N(C(=O)Nc2ccc(C(C)C)cc2)C1] | 4′-{[(4R)-4-Methoxy-1-{[4-(propan-2- yl)phenyl]carbamoyl}-D-prolyl]amino}[1,1′- biphenyl]-4-carboxylic acid |
| 37A | 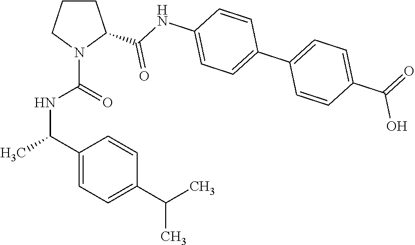 [SMILES: CC(C)c1ccc([C@H](C)NC(=O)N2CCC[C@@H]2C(=O)Nc2ccc(-c3ccc(C(=O)O)cc3)cc2)cc1] | 4′-{[1-({(1S)-1-[4-(propan-2- yl)phenyl]ethyl}carbamoyl)-D- prolyl]amino}[1,1′-biphenyl]-4-carboxylic acid |
| 38A | 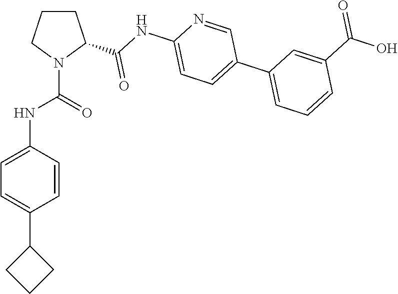 [SMILES: O=C(O)c1cccc(-c2ccc(NC(=O)[C@H]3CCCN3C(=O)Nc3ccc(C4CCC4)cc3)nc2)c1] | 3-[6-({1-[(4-cyclobutylphenyl)carbamoyl]-D- prolyl}amino)pyridin-3-yl]benzoic acid |
| 39A | 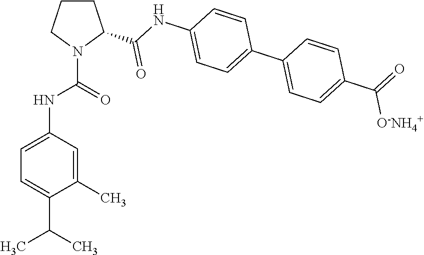 | ammonium 4′-[(1-{[3-methyl-4-(propan-2- yl)phenyl]carbamoyl}-D-prolyl)amino][1,1′- biphenyl]-4-carboxylate |
| 40A | 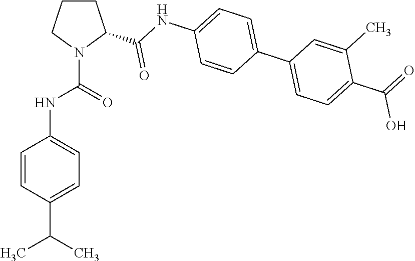 [SMILES: Cc1cc(-c2ccc(NC(=O)[C@H]3CCCN3C(=O)Nc3ccc(C(C)C)cc3)cc2)ccc1C(=O)O] | 3-methyl-4′-[(1-{[4-(propan-2- yl)phenyl]carbamoyl}-D-prolyl)amino][1,1′- biphenyl]-4-carboxylic acid |
| 41A | 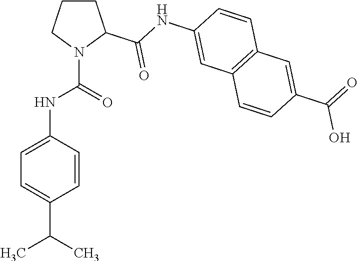 [SMILES: CC(C)c1ccc(NC(=O)N2CCCC2C(=O)Nc2ccc3cc(C(=O)O)ccc3c2)cc1] | 6-[(1-{[4-(propan-2- yl)phenyl]carbamoyl}prolyl)amino] naphthalene-2-carboxylic acid |
| 42A | 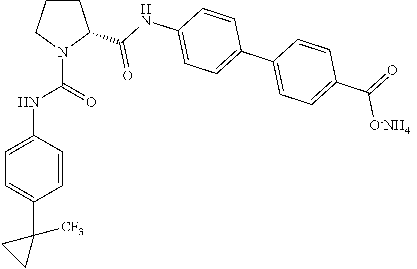 | ammonium 4′-{[1-({4-[1- (trifluoromethyl)cyclopropyl]phenyl} carbamoyl)-D-prolyl]amino}[1,1′-biphenyl]-4- carboxylate |
| 43A | 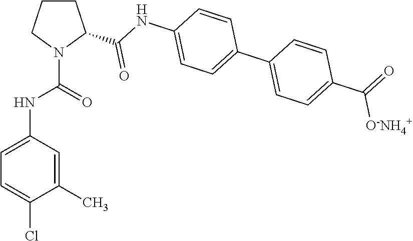 | ammonium 4′-({1-[(4-chloro-3- methylphenyl)carbamoyl]-D- prolyl}amino)[1,1′-biphenyl]-4-carboxylate |
| 44A | 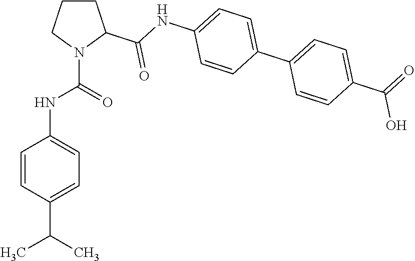 [SMILES: CC(C)c1ccc(NC(=O)N2CCCC2C(=O)Nc2ccc(-c3ccc(C(=O)O)cc3)cc2)cc1] | 4′-[(1-{[4-(propan-2- yl)phenyl]carbamoyl}prolyl)amino][1,1′- biphenyl]-4-carboxylic acid |
| 45A | 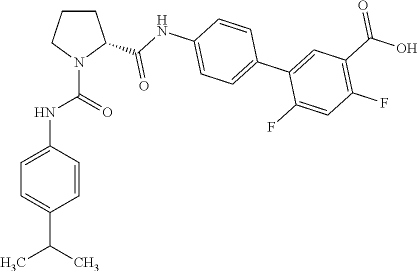 [SMILES: CC(C)c1ccc(NC(=O)N2CCC[C@@H]2C(=O)Nc2ccc(-c3cc(C(=O)O)c(F)cc3F)cc2)cc1] | 4,6-difluoro-4′-[(1-{[4-(propan-2- yl)phenyl]carbamoyl}-D-prolyl)amino][1,1′- biphenyl]-3-carboxylic acid |
| 46A | 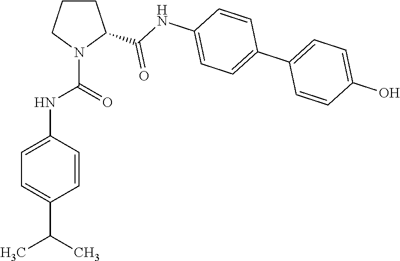 [SMILES: CC(C)c1ccc(NC(=O)N2CCC[C@@H]2C(=O)Nc2ccc(-c3ccc(O)cc3)cc2)cc1] | (2R)-N²-(4′-hydroxy[1,1′-biphenyl]-4-yl)-N¹- [4-(propan-2-yl)phenyl]pyrrolidine-1,2- dicarboxamide |
| 47A | 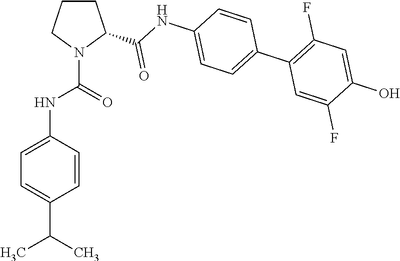 [SMILES: CC(C)c1ccc(NC(=O)N2CCC[C@@H]2C(=O)Nc2ccc(-c3cc(F)c(O)cc3F)cc2)cc1] | (2R)-N²-(2′,5′-difluoro-4′-hydroxy[1,1′- biphenyl]-4-yl)-N¹-[4-(propan-2- yl)phenyl]pyrrolidine-1,2-dicarboxamide |
| 48A | 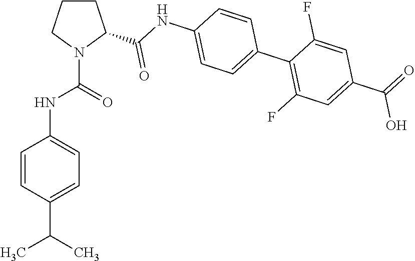 [SMILES: CC(C)c1ccc(NC(=O)N2CCC[C@@H]2C(=O)Nc2ccc(-c3c(F)cc(C(=O)O)cc3F)cc2)cc1] | 2,6-difluoro-4′-[(1-{[4-(propan-2- yl)phenyl]carbamoyl}-D-prolyl)amino][1,1′- biphenyl]-4-carboxylic acid |
| 49A | 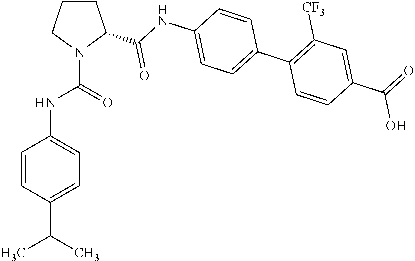 [SMILES: CC(C)c1ccc(NC(=O)N2CCC[C@@H]2C(=O)Nc2ccc(-c3ccc(C(=O)O)cc3C(F)(F)F)cc2)cc1] | 4′-[(1-{[4-(propan-2-yl)phenyl]carbamoyl}- D-prolyl)amino]-2-(trifluoromethyl)[1,1′- biphenyl]-4-carboxylic acid |
| 50A | 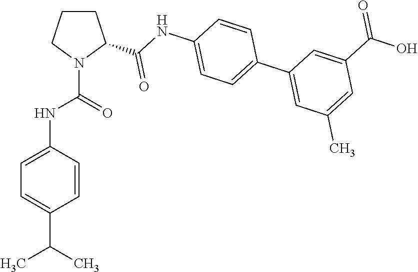 [SMILES: Cc1cc(C(=O)O)cc(-c2ccc(NC(=O)[C@H]3CCCN3C(=O)Nc3ccc(C(C)C)cc3)cc2)c1] | 5-methyl-4′-[(1-{[4-(propan-2- yl)phenyl]carbamoyl}-D-prolyl)amino][1,1′- biphenyl]-3-carboxylic acid |
| 51A | 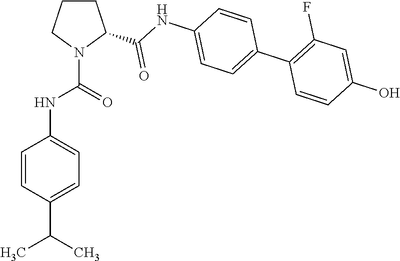 [SMILES: CC(C)c1ccc(NC(=O)N2CCC[C@@H]2C(=O)Nc2ccc(-c3ccc(O)cc3F)cc2)cc1] | (2R)-N²-(2′-fluoro-4′-hydroxy[1,1′-biphenyl]- 4-yl)-N¹-[4-(propan-2-yl)phenyl]pyrrolidine- 1,2-dicarboxamide |
| 52A | 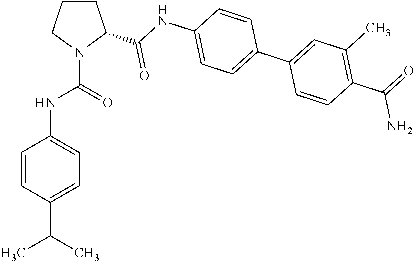 [SMILES: Cc1cc(-c2ccc(NC(=O)[C@H]3CCCN3C(=O)Nc3ccc(C(C)C)cc3)cc2)ccc1C(N)=O] | (2R)-N²-(4′-carbamoyl-3′-methyl[1,1′- biphenyl]-4-yl)-N¹-[4-(propan-2- yl)phenyl]pyrrolidine-1,2-dicarboxamide |
| 53A | 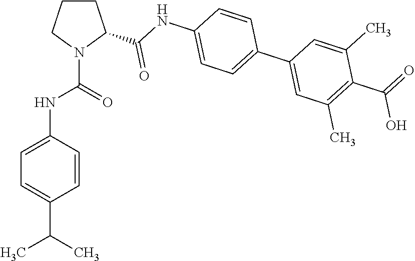 [SMILES: Cc1cc(-c2ccc(NC(=O)[C@H]3CCCN3C(=O)Nc3ccc(C(C)C)cc3)cc2)cc(C)c1C(=O)O] | 3,5-dimethyl-4′-[(1-{[4-(propan-2- yl)phenyl]carbamoyl}-D-prolyl)amino][1,1′- biphenyl]-4-carboxylic acid |
| 54A | 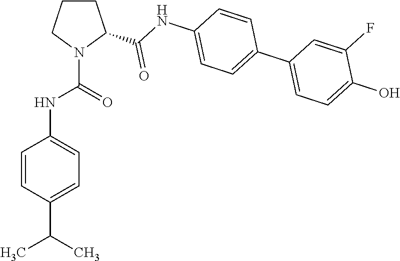 [SMILES: CC(C)c1ccc(NC(=O)N2CCC[C@@H]2C(=O)Nc2ccc(-c3ccc(O)c(F)c3)cc2)cc1] | (2R)-N²-(3′-fluoro-4′-hydroxy[1,1′-biphenyl]- 4-yl)-N¹-[4-(propan-2-yl)phenyl]pyrrolidine- 1,2-dicarboxamide |
| 55A | 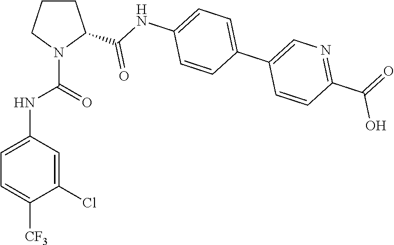 [SMILES: O=C(O)c1ccc(-c2ccc(NC(=O)[C@H]3CCCN3C(=O)Nc3ccc(C(F)(F)F)c(Cl)c3)cc2)cn1] | 5-{4-[(1-{[3-chloro-4- (trifluoromethyl)phenyl]carbamoyl}-D- prolyl)amino]phenyl}pyridine-2-carboxylic acid |
| 56A | 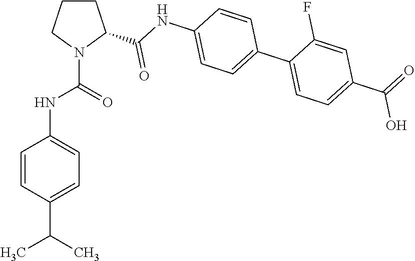 [SMILES: CC(C)c1ccc(NC(=O)N2CCC[C@@H]2C(=O)Nc2ccc(-c3ccc(C(=O)O)cc3F)cc2)cc1] | 2-fluoro-4′-[(1-{[4-(propan-2- yl)phenyl]carbamoyl}-D-prolyl)amino][1,1′- biphenyl]-4-carboxylic acid |
| 57A | 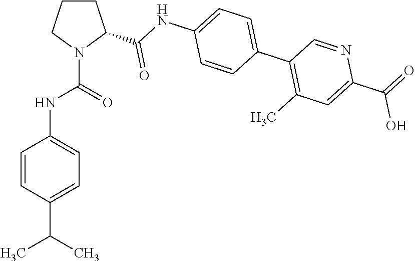 [SMILES: Cc1cc(C(=O)O)ncc1-c1ccc(NC(=O)[C@H]2CCCN2C(=O)Nc2ccc(C(C)C)cc2)cc1] | 4-methyl-5-{4-[(1-{[4-(propan-2- yl)phenyl]carbamoyl}-D- prolyl)amino]phenyl}pyridine-2-carboxylic acid |
| 58A | 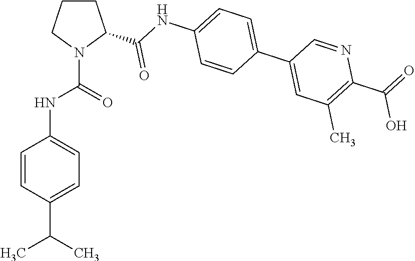 [SMILES: Cc1cc(-c2ccc(NC(=O)[C@H]3CCCN3C(=O)Nc3ccc(C(C)C)cc3)cc2)cnc1C(=O)O] | 3-methyl-5-{4-[(1-{[4-(propan-2- yl)phenyl]carbamoyl}-D- prolyl)amino]phenyl}pyridine-2-carboxylic acid |
| 59A | 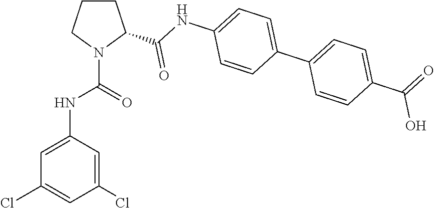 [SMILES: O=C(O)c1ccc(-c2ccc(NC(=O)[C@H]3CCCN3C(=O)Nc3cc(Cl)cc(Cl)c3)cc2)cc1] | 4′-({1-[(3,5-dichlorophenyl)carbamoyl]-D- prolyl}amino)[1,1′-biphenyl]-4-carboxylic acid |
| 60A | 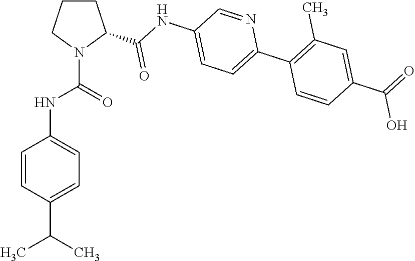 [SMILES: Cc1cc(C(=O)O)ccc1-c1ccc(NC(=O)[C@H]2CCCN2C(=O)Nc2ccc(C(C)C)cc2)cn1] | 3-methyl-4-{5-[(1-{[4-(propan-2- yl)phenyl]carbamoyl}-D- prolyl)amino]pyridin-2-yl}benzoic acid |
| 61A | 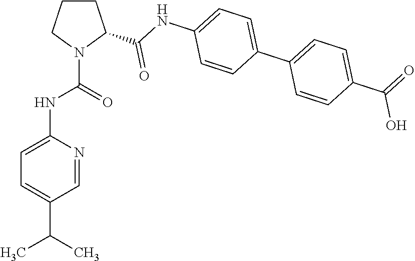 [SMILES: CC(C)c1ccc(NC(=O)N2CCC[C@@H]2C(=O)Nc2ccc(-c3ccc(C(=O)O)cc3)cc2)nc1] | 4′-[(1-{[5-(propan-2-yl)pyridin-2- yl]carbamoyl}-D-prolyl)amino][1,1′- biphenyl]-4-carboxylic acid |
| 62A | 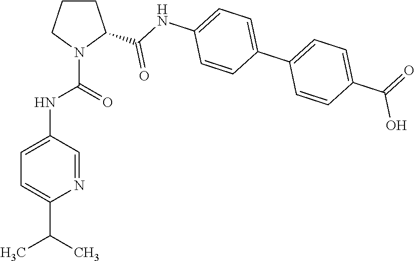 [SMILES: CC(C)c1ccc(NC(=O)N2CCC[C@@H]2C(=O)Nc2ccc(-c3ccc(C(=O)O)cc3)cc2)cn1] | 4′-[(1-{[6-(propan-2-yl)pyridin-3- yl]carbamoyl}-D-prolyl)amino][1,1′- biphenyl]-4-carboxylic acid |
| 63A | 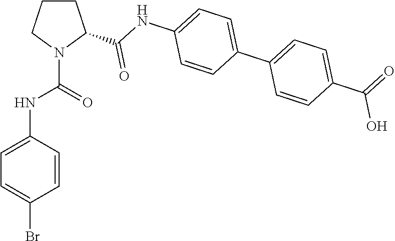 [SMILES: O=C(O)c1ccc(-c2ccc(NC(=O)[C@H]3CCCN3C(=O)Nc3ccc(Br)cc3)cc2)cc1] | 4′-({1-[(4-bromophenyl)carbamoyl]-D- prolyl}amino)[1,1′-biphenyl]-4-carboxylic acid |
| 64A | 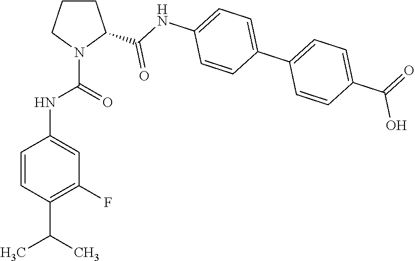 [SMILES: CC(C)c1ccc(NC(=O)N2CCC[C@@H]2C(=O)Nc2ccc(-c3ccc(C(=O)O)cc3)cc2)cc1F] | 4′-[(1-{[3-fluoro-4-(propan-2- yl)phenyl]carbamoyl}-D-prolyl)amino][1,1′- biphenyl]-4-carboxylic acid |
| 65A | 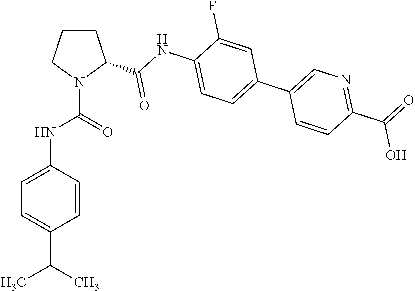 [SMILES: CC(C)c1ccc(NC(=O)N2CCC[C@@H]2C(=O)Nc2ccc(-c3ccc(C(=O)O)nc3)cc2F)cc1] | 5-{3-fluoro-4-[(1-{[4-(propan-2- yl)phenyl]carbamoyl}-D- prolyl)amino]phenyl}pyridine-2-carboxylic acid |
| 66A | 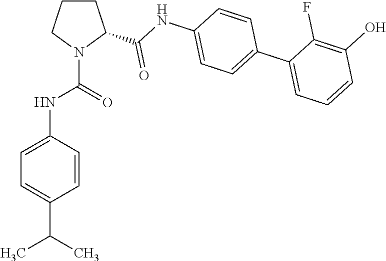 [SMILES: CC(C)c1ccc(NC(=O)N2CCC[C@@H]2C(=O)Nc2ccc(-c3cccc(O)c3F)cc2)cc1] | (2R)-N²-(2′-fluoro-3′-hydroxy[1,1′-biphenyl]- 4-yl)-N¹-[4-(propan-2-yl)phenyl]pyrrolidine- 1,2-dicarboxamide |
| 67A | 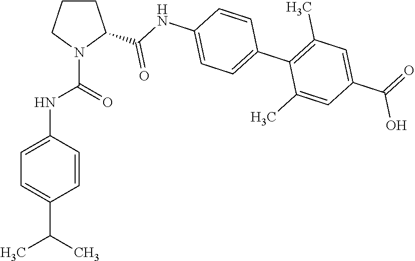 [SMILES: Cc1cc(C(=O)O)cc(C)c1-c1ccc(NC(=O)[C@H]2CCCN2C(=O)Nc2ccc(C(C)C)cc2)cc1] | 2,6-dimethyl-4′-[(1-{[4-(propan-2- yl)phenyl]carbamoyl}-D-prolyl)amino][1,1′- biphenyl]-4-carboxylic acid |
| 68A | 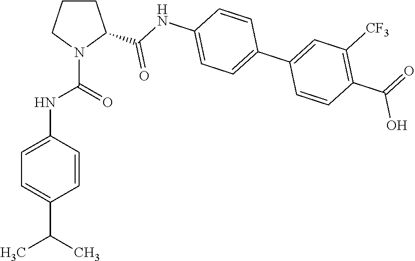 [SMILES: CC(C)c1ccc(NC(=O)N2CCC[C@@H]2C(=O)Nc2ccc(-c3ccc(C(=O)O)c(C(F)(F)F)c3)cc2)cc1] | 4′-[(1-{[4-(propan-2-yl)phenyl]carbamoyl}- D-prolyl)amino]-3-(trifluoromethyl)[1,1′- biphenyl]-4-carboxylic acid |
| 69A | 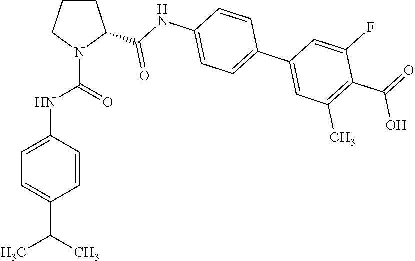 [SMILES: Cc1cc(-c2ccc(NC(=O)[C@H]3CCCN3C(=O)Nc3ccc(C(C)C)cc3)cc2)cc(F)c1C(=O)O] | 3-fluoro-5-methyl-4′-[(1-{[4-(propan-2- yl)phenyl]carbamoyl}-D-prolyl)amino][1,1′- biphenyl]-4-carboxylic acid |
| 70A | 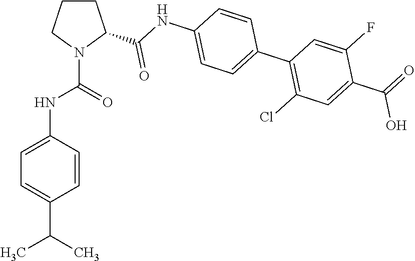 [SMILES: CC(C)c1ccc(NC(=O)N2CCC[C@@H]2C(=O)Nc2ccc(-c3cc(F)c(C(=O)O)cc3Cl)cc2)cc1] | 2-chloro-5-fluoro-4′-[(1-{[4-(propan-2- yl)phenyl]carbamoyl}-D-prolyl)amino][1,1′- biphenyl]-4-carboxylic acid |
| 71A | 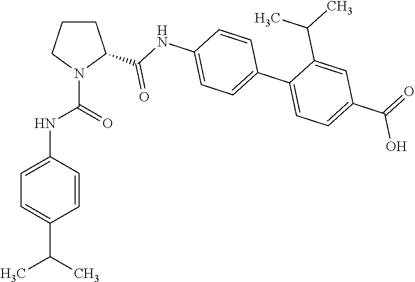 [SMILES: CC(C)c1ccc(NC(=O)N2CCC[C@@H]2C(=O)Nc2ccc(-c3ccc(C(=O)O)cc3C(C)C)cc2)cc1] | 2-(propan-2-yl)-4′-[(1-{[4-(propan-2- yl)phenyl]carbamoyl}-D-prolyl)amino][1,1′- biphenyl]-4-carboxylic acid |
| 72A | 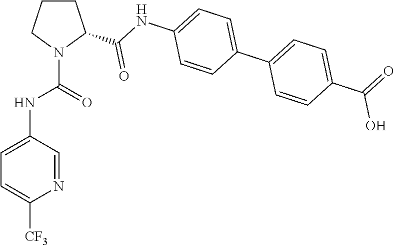 [SMILES: O=C(O)c1ccc(-c2ccc(NC(=O)[C@H]3CCCN3C(=O)Nc3ccc(C(F)(F)F)nc3)cc2)cc1] | 4′-[(1-{[6-(trifluoromethyl)pyridin-3- yl]carbamoyl}-D-prolyl)amino][1,1′- biphenyl]-4-carboxylic acid |
| 73A | 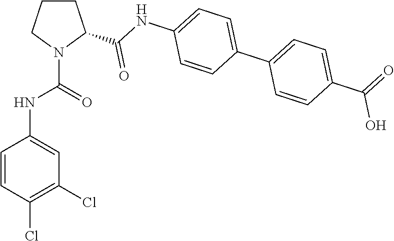 [SMILES: O=C(O)c1ccc(-c2ccc(NC(=O)[C@H]3CCCN3C(=O)Nc3ccc(Cl)c(Cl)c3)cc2)cc1] | 4′-({1-[(3,4-dichlorophenyl)carbamoyl]-D- prolyl}amino)[1,1′-biphenyl]-4-carboxylic acid |
| 74A | 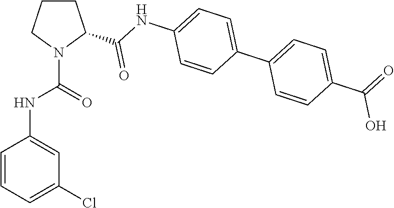 [SMILES: O=C(O)c1ccc(-c2ccc(NC(=O)[C@H]3CCCN3C(=O)Nc3cccc(Cl)c3)cc2)cc1] | 4′-({1-[(3-chlorophenyl)carbamoyl]-D- prolyl}amino)[1,1′-biphenyl]-4-carboxylic acid |
| 75A | 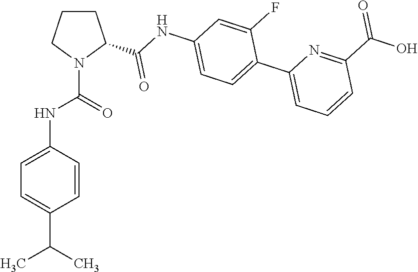 [SMILES: CC(C)c1ccc(NC(=O)N2CCC[C@@H]2C(=O)Nc2ccc(-c3cccc(C(=O)O)n3)c(F)c2)cc1] | 6-{2-fluoro-4-[(1-{[4-(propan-2- yl)phenyl]carbamoyl}-D- prolyl)amino]phenyl}pyridine-2-carboxylic acid |
| 76A | 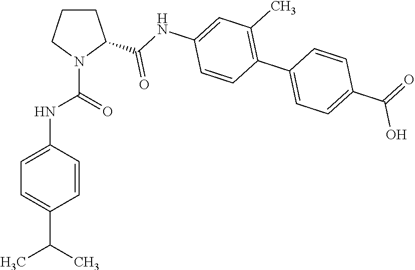 [SMILES: Cc1cc(NC(=O)[C@H]2CCCN2C(=O)Nc2ccc(C(C)C)cc2)ccc1-c1ccc(C(=O)O)cc1] | 2′-methyl-4′-[(1-{[4-(propan-2- yl)phenyl]carbamoyl}-D-prolyl)amino][1,1′- biphenyl]-4-carboxylic acid |
| 77A | 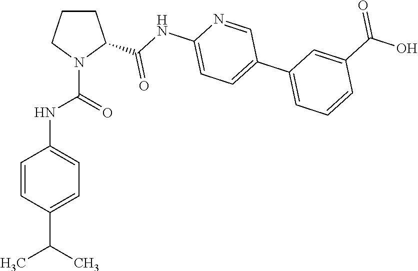 [SMILES: CC(C)c1ccc(NC(=O)N2CCC[C@@H]2C(=O)Nc2ccc(-c3cccc(C(=O)O)c3)cn2)cc1] | 3-{6-[(1-{[4-(propan-2- yl)phenyl]carbamoyl}-D- prolyl)amino]pyridin-3-yl}benzoic acid |
| 78A | 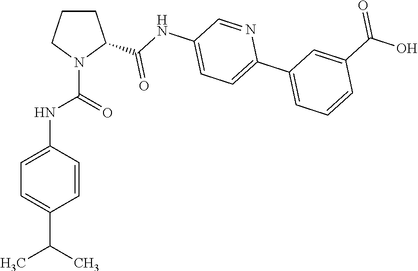 [SMILES: CC(C)c1ccc(NC(=O)N2CCC[C@@H]2C(=O)Nc2ccc(-c3cccc(C(=O)O)c3)nc2)cc1] | 3-{5-[(1-{[4-(propan-2- yl)phenyl]carbamoyl}-D- prolyl)amino]pyridin-2-yl}benzoic acid |
| 79A | 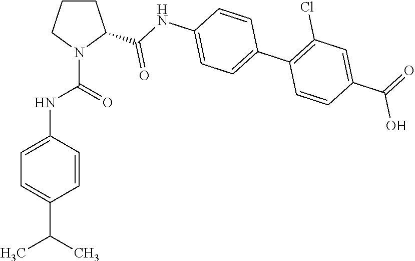 [SMILES: CC(C)c1ccc(NC(=O)N2CCC[C@@H]2C(=O)Nc2ccc(-c3ccc(C(=O)O)cc3Cl)cc2)cc1] | 2-chloro-4′-[(1-{[4-(propan-2- yl)phenyl]carbamoyl}-D-prolyl)amino][1,1′- biphenyl]-4-carboxylic acid |
| 80A | 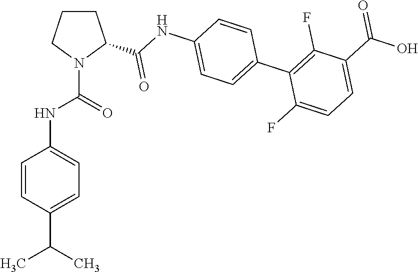 [SMILES: CC(C)c1ccc(NC(=O)N2CCC[C@@H]2C(=O)Nc2ccc(-c3c(F)ccc(C(=O)O)c3F)cc2)cc1] | 2,6-difluoro-4′-[(1-{[4-(propan-2- yl)phenyl]carbamoyl}-D-prolyl)amino][1,1′- biphenyl]-3-carboxylic acid |
| 81A | 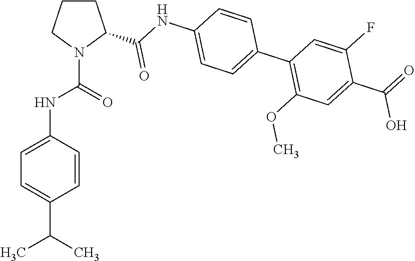 [SMILES: COc1cc(C(=O)O)c(F)cc1-c1ccc(NC(=O)[C@H]2CCCN2C(=O)Nc2ccc(C(C)C)cc2)cc1] | 5-fluoro-2-methoxy-4′-[(1-{[4-(propan-2- yl)phenyl]carbamoyl}-D-prolyl)amino][1,1′- biphenyl]-4-carboxylic acid |
| 82A | 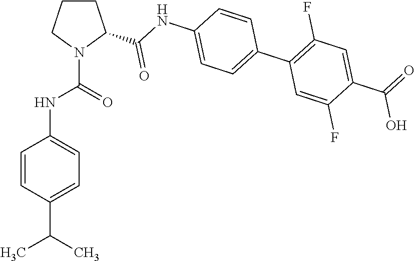 [SMILES: CC(C)c1ccc(NC(=O)N2CCC[C@@H]2C(=O)Nc2ccc(-c3cc(F)c(C(=O)O)cc3F)cc2)cc1] | 2,5-difluoro-4′-[(1-{[4-(propan-2- yl)phenyl]carbamoyl}-D-prolyl)amino][1,1′- biphenyl]-4-carboxylic acid |
| 83A | 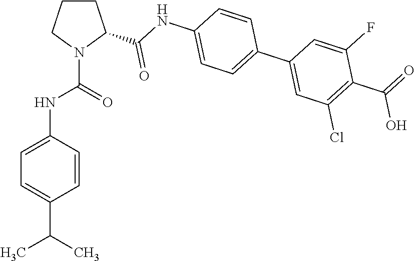 [SMILES: CC(C)c1ccc(NC(=O)N2CCC[C@@H]2C(=O)Nc2ccc(-c3cc(F)c(C(=O)O)c(Cl)c3)cc2)cc1] | 3-chloro-5-fluoro-4′-[(1-{[4-(propan-2- yl)phenyl]carbamoyl}-D-prolyl)amino][1,1′- biphenyl]-4-carboxylic acid |
| 84A | 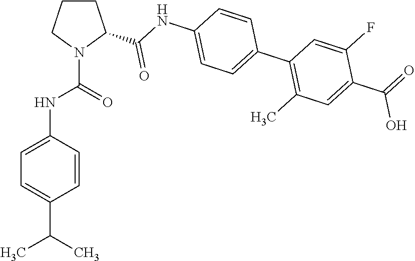 [SMILES: Cc1cc(C(=O)O)c(F)cc1-c1ccc(NC(=O)[C@H]2CCCN2C(=O)Nc2ccc(C(C)C)cc2)cc1] | 5-fluoro-2-methyl-4′-[(1-{[4-(propan-2- yl)phenyl]carbamoyl}-D-prolyl)amino][1,1′- biphenyl]-4-carboxylic acid |
| 85A | 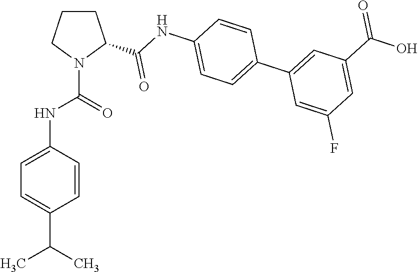 [SMILES: CC(C)c1ccc(NC(=O)N2CCC[C@@H]2C(=O)Nc2ccc(-c3cc(F)cc(C(=O)O)c3)cc2)cc1] | 5-fluoro-4′-[(1-{[4-(propan-2- yl)phenyl]carbamoyl}-D-prolyl)amino][1,1′- biphenyl]-3-carboxylic acid |
| 86A | 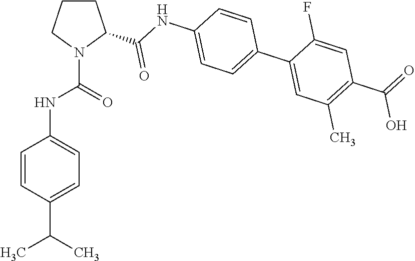 [SMILES: Cc1cc(-c2ccc(NC(=O)[C@H]3CCCN3C(=O)Nc3ccc(C(C)C)cc3)cc2)c(F)cc1C(=O)O] | 2-fluoro-5-methyl-4′-[(1-{[4-(propan-2- yl)phenyl]carbamoyl}-D-prolyl)amino][1,1′- biphenyl]-4-carboxylic acid |
| 87A | 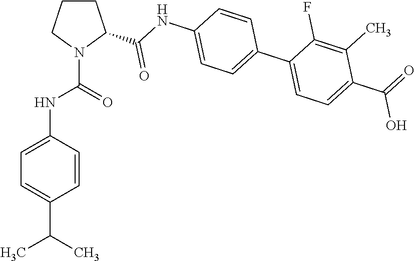 [SMILES: Cc1c(C(=O)O)ccc(-c2ccc(NC(=O)[C@H]3CCCN3C(=O)Nc3ccc(C(C)C)cc3)cc2)c1F] | 2-fluoro-3-methyl-4′-[(1-{[4-(propan-2- yl)phenyl]carbamoyl}-D-prolyl)amino][1,1′- biphenyl]-4-carboxylic acid |
| 88A | 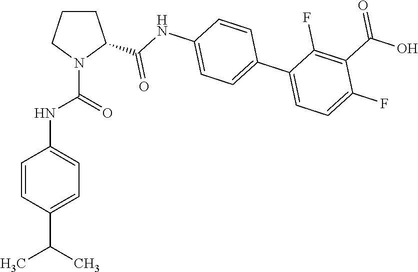 [SMILES: CC(C)c1ccc(NC(=O)N2CCC[C@@H]2C(=O)Nc2ccc(-c3ccc(F)c(C(=O)O)c3F)cc2)cc1] | 2,4-difluoro-4′-[(1-{[4-(propan-2- yl)phenyl]carbamoyl}-D-prolyl)amino][1,1′- biphenyl]-3-carboxylic acid |
| 89A | 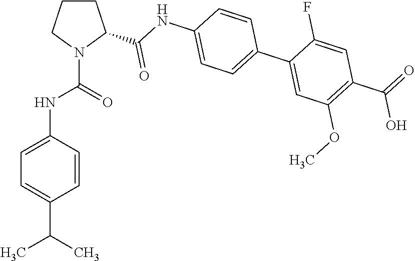 [SMILES: COc1cc(-c2ccc(NC(=O)[C@H]3CCCN3C(=O)Nc3ccc(C(C)C)cc3)cc2)c(F)cc1C(=O)O] | 2-fluoro-5-methoxy-4′-[(1-{[4-(propan-2- yl)phenyl]carbamoyl}-D-prolyl)amino][1,1′- biphenyl]-4-carboxylic acid |
| 90A | 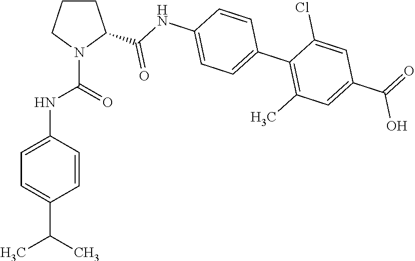 [SMILES: Cc1cc(C(=O)O)cc(Cl)c1-c1ccc(NC(=O)[C@H]2CCCN2C(=O)Nc2ccc(C(C)C)cc2)cc1] | 2-chloro-6-methyl-4′-[(1-{[4-(propan-2- yl)phenyl]carbamoyl}-D-prolyl)amino][1,1′- biphenyl]-4-carboxylic acid |
| 91A | 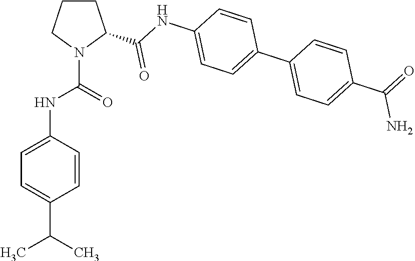 [SMILES: CC(C)c1ccc(NC(=O)N2CCC[C@@H]2C(=O)Nc2ccc(-c3ccc(C(N)=O)cc3)cc2)cc1] | (2R)-N²-(4′-carbamoyl[1,1′-biphenyl]-4-yl)- N¹-[4-(propan-2-yl)phenyl]pyrrolidine-1,2- dicarboxamide |
| 92A | 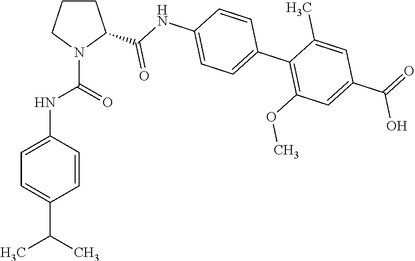 [SMILES: COc1cc(C(=O)O)cc(C)c1-c1ccc(NC(=O)[C@H]2CCCN2C(=O)Nc2ccc(C(C)C)cc2)cc1] | 2-methoxy-6-methyl-4′-[(1-{[4-(propan-2- yl)phenyl]carbamoyl}-D-prolyl)amino][1,1′- biphenyl]-4-carboxylic acid |
| 93A | 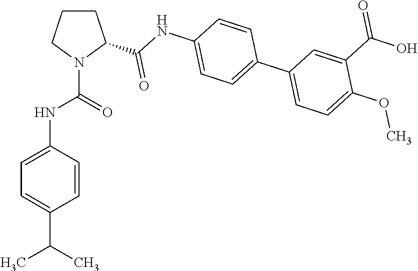 [SMILES: COc1ccc(-c2ccc(NC(=O)[C@H]3CCCN3C(=O)Nc3ccc(C(C)C)cc3)cc2)cc1C(=O)O] | 4-methoxy-4′-[(1-{[4-(propan-2- yl)phenyl]carbamoyl}-D-prolyl)amino][1,1′- biphenyl]-3-carboxylic acid |
| 94A | 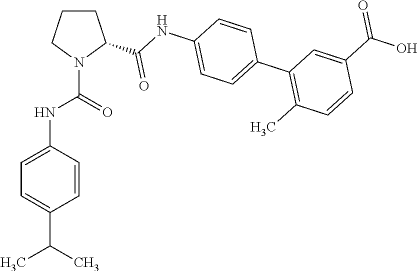 [SMILES: Cc1ccc(C(=O)O)cc1-c1ccc(NC(=O)[C@H]2CCCN2C(=O)Nc2ccc(C(C)C)cc2)cc1] | 6-methyl-4′-[(1-{[4-(propan-2- yl)phenyl]carbamoyl}-D-prolyl)amino][1,1′- biphenyl]-3-carboxylic acid |
| 95A | 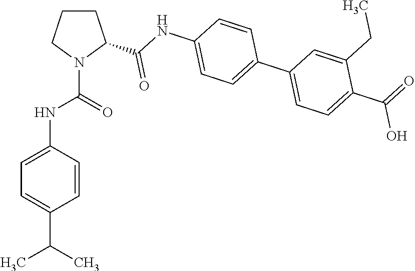 [SMILES: CCc1cc(-c2ccc(NC(=O)[C@H]3CCCN3C(=O)Nc3ccc(C(C)C)cc3)cc2)ccc1C(=O)O] | 3-ethyl-4′-[(1-{[4-(propan-2- yl)phenyl]carbamoyl}-D-prolyl)amino][1,1′- biphenyl]-4-carboxylic acid |
| 96A | 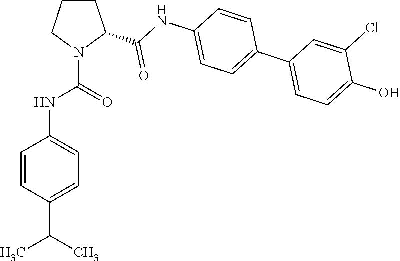 [SMILES: CC(C)c1ccc(NC(=O)N2CCC[C@@H]2C(=O)Nc2ccc(-c3ccc(O)c(Cl)c3)cc2)cc1] | (2R)-N²-(3′-chloro-4′-hydroxy[1,1′- biphenyl]-4-yl)-N¹-[4-(propan-2- yl)phenyl]pyrrolidine-1,2-dicarboxamide |
| 97A | 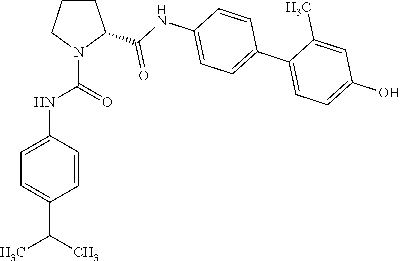 [SMILES: Cc1cc(O)ccc1-c1ccc(NC(=O)[C@H]2CCCN2C(=O)Nc2ccc(C(C)C)cc2)cc1] | (2R)-N²-(4′-hydroxy-2′-methyl[1,1′- biphenyl]-4-yl)-N¹-[4-(propan-2- yl)phenyl]pyrrolidine-1,2-dicarboxamide |
| 98A | 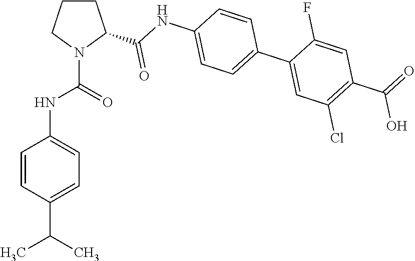 [SMILES: CC(C)c1ccc(NC(=O)N2CCC[C@@H]2C(=O)Nc2ccc(-c3cc(Cl)c(C(=O)O)cc3F)cc2)cc1] | 5-chloro-2-fluoro-4′-[(1-{[4-(propan-2- yl)phenyl]carbamoyl}-D-prolyl)amino][1,1′- biphenyl]-4-carboxylic acid |
| 99A | 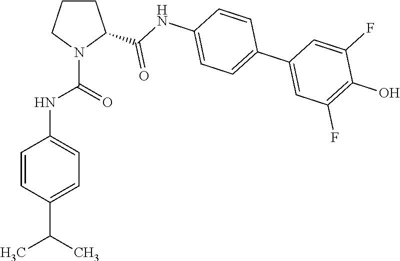 [SMILES: CC(C)c1ccc(NC(=O)N2CCC[C@@H]2C(=O)Nc2ccc(-c3cc(F)c(O)c(F)c3)cc2)cc1] | (2R)-N²-(3′,5′-difluoro-4′-hydroxy[1,1′- biphenyl]-4-yl)-N¹-[4-(propan-2- yl)phenyl]pyrrolidine-1,2-dicarboxamide |
| 100A | 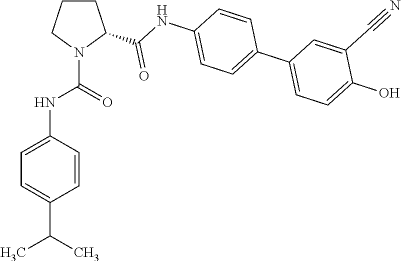 [SMILES: CC(C)c1ccc(NC(=O)N2CCC[C@@H]2C(=O)Nc2ccc(-c3ccc(O)c(C#N)c3)cc2)cc1] | (2R)-N²-(3′-cyano-4′-hydroxy[1,1′- biphenyl]-4-yl)-N¹-[4-(propan-2- yl)phenyl]pyrrolidine-1,2-dicarboxamide |
| 101A | 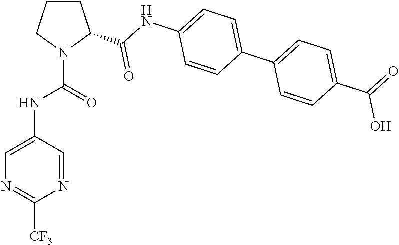 [SMILES: O=C(O)c1ccc(-c2ccc(NC(=O)[C@H]3CCCN3C(=O)Nc3cnc(C(F)(F)F)nc3)cc2)cc1] | 4′-[(1-{[2-(trifluoromethyl)pyrimidin-5- yl]carbamoyl}-D-prolyl)amino][1,1′- biphenyl]-4-carboxylic acid |
| 102A | 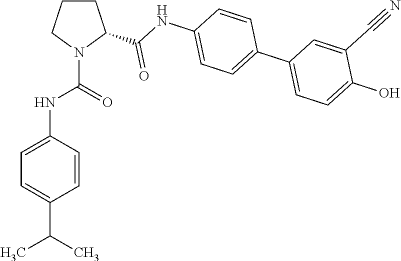 [SMILES: CC(C)c1ccc(NC(=O)N2CCC[C@@H]2C(=O)Nc2ccc(-c3ccc(O)c(C#N)c3)cc2)cc1] | 4′-[(1-{[6-methyl-5-(propan-2-yl)pyridin-2- yl]carbamoyl}-D-prolyl)amino][1,1′- biphenyl]-4-carboxylic acid |
| 103A | 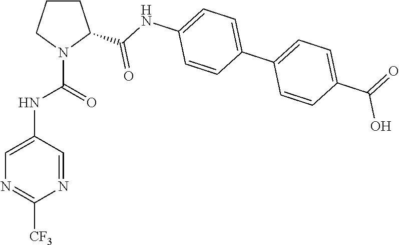 [SMILES: O=C(O)c1ccc(-c2ccc(NC(=O)[C@H]3CCCN3C(=O)Nc3cnc(C(F)(F)F)nc3)cc2)cc1] | 4′-({1-[(2,4-dichlorophenyl)carbamoyl]-D- prolyl}amino)[1,1′-biphenyl]-4-carboxylic acid |
| 104A | 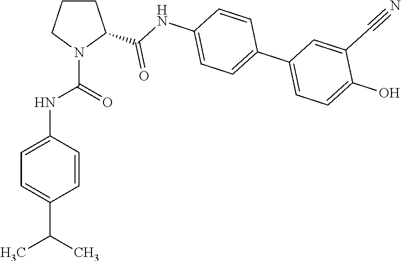 [SMILES: CC(C)c1ccc(NC(=O)N2CCC[C@@H]2C(=O)Nc2ccc(-c3ccc(O)c(C#N)c3)cc2)cc1] | 4′-{[(1R,2R,5S)-3-{[4-(propan-2- yl)phenyl]carbamoyl}-3- azabicyclo[3.1.0]hexane-2- carbonyl]amino}[1,1′-biphenyl]-4-carboxylic acid |
| 105A | 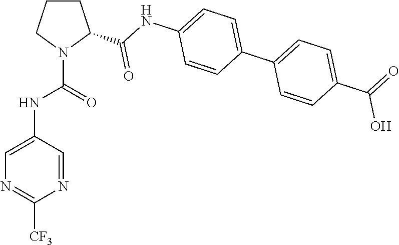 [SMILES: O=C(O)c1ccc(-c2ccc(NC(=O)[C@H]3CCCN3C(=O)Nc3cnc(C(F)(F)F)nc3)cc2)cc1] | 4′-{[(1S,2R,5R)-3-{[4-(propan-2- yl)phenyl]carbamoyl}-3- azabicyclo[3.1.0]hexane-2- carbonyl]amino}[1,1′-biphenyl]-4-carboxylic acid |
| 106A | 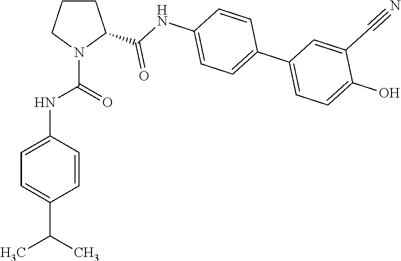 [SMILES: CC(C)c1ccc(NC(=O)N2CCC[C@@H]2C(=O)Nc2ccc(-c3ccc(O)c(C#N)c3)cc2)cc1] | 4′-[(1-{[3-methyl-4- (trifluoromethyl)phenyl]carbamoyl}-D- prolyl)amino][1,1′-biphenyl]-4-carboxylic acid |
| 107A | 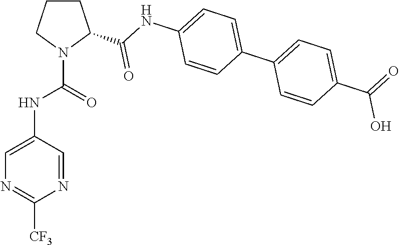 [SMILES: O=C(O)c1ccc(-c2ccc(NC(=O)[C@H]3CCCN3C(=O)Nc3cnc(C(F)(F)F)nc3)cc2)cc1] | 4′-[(1-{[(4-chloro-3- cyanophenyl)methyl]carbamoyl}-D- prolyl)amino][1,1′-biphenyl]-4-carboxylic acid |
| 108A | 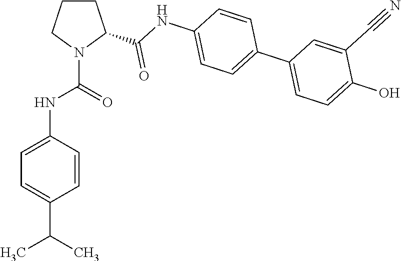 [SMILES: CC(C)c1ccc(NC(=O)N2CCC[C@@H]2C(=O)Nc2ccc(-c3ccc(O)c(C#N)c3)cc2)cc1] | 4′-{[1-({[2-(trifluoromethyl)pyrimidin-5- yl]methyl}carbamoyl)-D-prolyl]amino}[1,1′- biphenyl]-4-carboxylic acid |
| 109A | 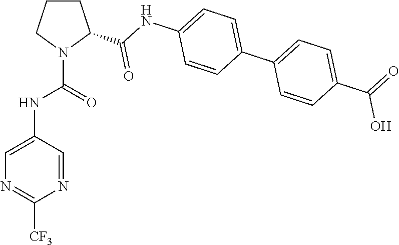 [SMILES: O=C(O)c1ccc(-c2ccc(NC(=O)[C@H]3CCCN3C(=O)Nc3cnc(C(F)(F)F)nc3)cc2)cc1] | 4′-{[1-({[4-(propan-2- yl)phenyl]methyl}carbamoyl)-D- prolyl]amino}[1,1′-biphenyl]-4-carboxylic acid |
| 110A | 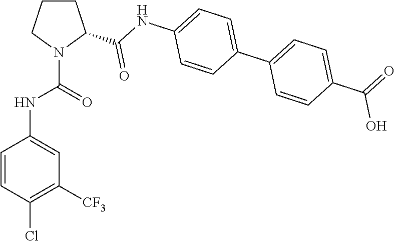 [SMILES: O=C(O)c1ccc(-c2ccc(NC(=O)[C@H]3CCCN3C(=O)Nc3ccc(Cl)c(C(F)(F)F)c3)cc2)cc1] | 4′-[(1-{[4-chloro-3- (trifluoromethyl)phenyl]carbamoyl}-D- prolyl)amino][1,1′-biphenyl]-4-carboxylic acid |
| 111A | 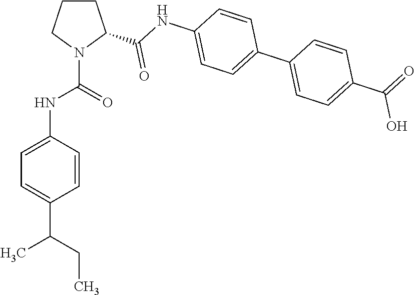 [SMILES: CCC(C)c1ccc(NC(=O)N2CCC[C@@H]2C(=O)Nc2ccc(-c3ccc(C(=O)O)cc3)cc2)cc1] | 4′-[(1-{[4-(butan-2-yl)phenyl]carbamoyl}-D- prolyl)amino][1,1′-biphenyl]-4-carboxylic acid |
| 112A | 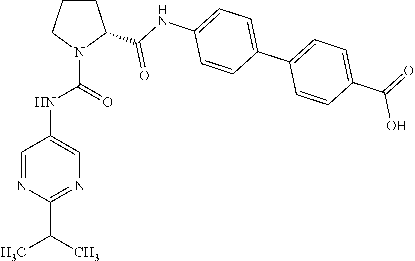 [SMILES: CC(C)c1ncc(NC(=O)N2CCC[C@@H]2C(=O)Nc2ccc(-c3ccc(C(=O)O)cc3)cc2)cn1] | 4′-[(1-{[2-(propan-2-yl)pyrimidin-5- yl]carbamoyl}-D-prolyl)amino][1,1′- biphenyl]-4-carboxylic acid |
| 113A | 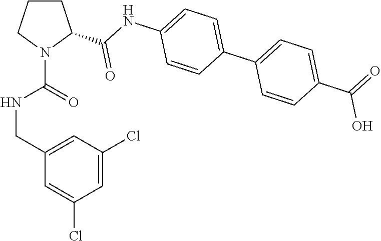 [SMILES: O=C(O)c1ccc(-c2ccc(NC(=O)[C@H]3CCCN3C(=O)NCc3cc(Cl)cc(Cl)c3)cc2)cc1] | 4′-[(1-{[(3,5- dichlorophenyl)methyl]carbamoyl}-D- prolyl)amino][1,1′-biphenyl]-4-carboxylic acid |
| 114A | 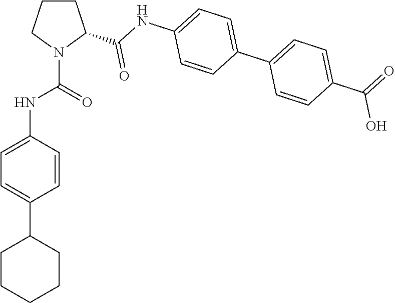 [SMILES: O=C(O)c1ccc(-c2ccc(NC(=O)[C@H]3CCCN3C(=O)Nc3ccc(C4CCCCC4)cc3)cc2)cc1] | 4′-({1-[(4-cyclohexylphenyl)carbamoyl]-D- prolyl}amino)[1,1′-biphenyl]-4-carboxylic acid |
| 115A | 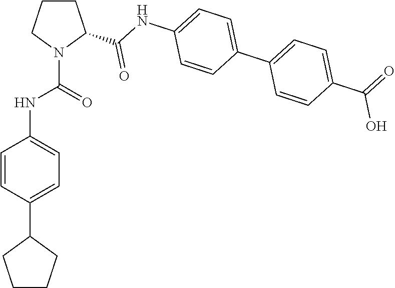 [SMILES: O=C(O)c1ccc(-c2ccc(NC(=O)[C@H]3CCCN3C(=O)Nc3ccc(C4CCCC4)cc3)cc2)cc1] | 4′-({1-[(4-cyclopentylphenyl)carbamoyl]-D- prolyl}amino)[1,1′-biphenyl]-4-carboxylic acid |
| 116A | 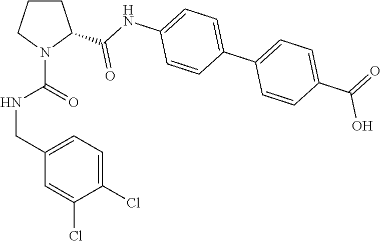 [SMILES: O=C(O)c1ccc(-c2ccc(NC(=O)[C@H]3CCCN3C(=O)NCc3ccc(Cl)c(Cl)c3)cc2)cc1] | 4′-[(1-{[(3,4- dichlorophenyl)methyl]carbamoyl}-D- prolyl)amino][1,1′-biphenyl]-4-carboxylic acid |
| 117A | 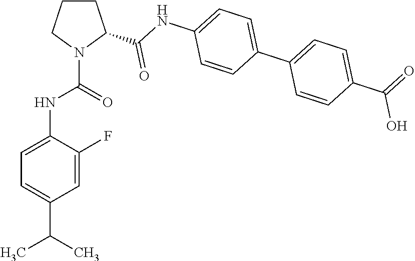 [SMILES: CC(C)c1ccc(NC(=O)N2CCC[C@@H]2C(=O)Nc2ccc(-c3ccc(C(=O)O)cc3)cc2)c(F)c1] | 4′-[(1-{[2-fluoro-4-(propan-2- yl)phenyl]carbamoyl}-D-prolyl)amino][1,1′- biphenyl]-4-carboxylic acid |
| 118A | 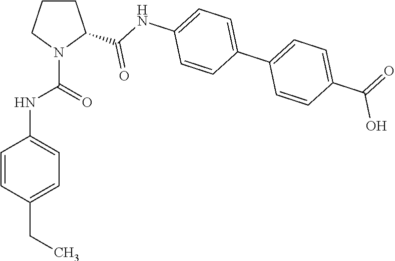 [SMILES: CCc1ccc(NC(=O)N2CCC[C@@H]2C(=O)Nc2ccc(-c3ccc(C(=O)O)cc3)cc2)cc1] | 4′-({1-[(4-ethylphenyl)carbamoyl]-D- prolyl}amino)[1,1′-biphenyl]-4-carboxylic acid |
| 119A | 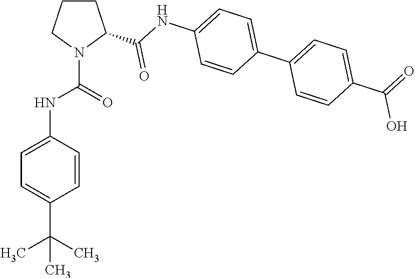 [SMILES: CC(C)(C)c1ccc(NC(=O)N2CCC[C@@H]2C(=O)Nc2ccc(-c3ccc(C(=O)O)cc3)cc2)cc1] | 4′-({1-[(4-tert-butylphenyl)carbamoyl]-D- prolyl}amino)[1,1′-biphenyl]-4-carboxylic acid |
| 120A | 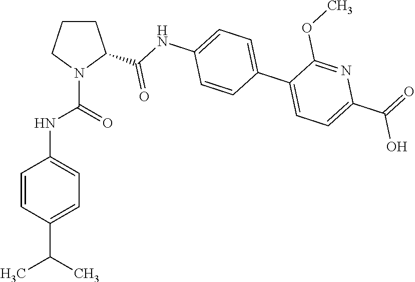 [SMILES: COc1nc(C(=O)O)ccc1-c1ccc(NC(=O)[C@H]2CCCN2C(=O)Nc2ccc(C(C)C)cc2)cc1] | 6-methoxy-5-{4-[(1-{[4-(propan-2- yl)phenyl]carbamoyl}-D- prolyl)amino]phenyl}pyridine-2-carboxylic acid |
| 121A | 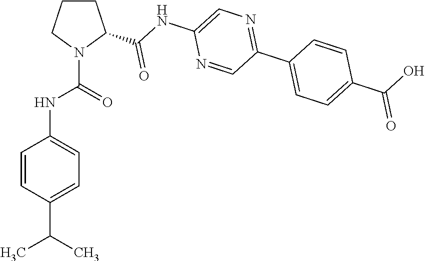 [SMILES: CC(C)c1ccc(NC(=O)N2CCC[C@@H]2C(=O)Nc2cnc(-c3ccc(C(=O)O)cc3)cn2)cc1] | 4-{5-[(1-{[4-(propan-2- yl)phenyl]carbamoyl}-D- prolyl)amino]pyrazin-2-yl}benzoic acid |
| 122A | 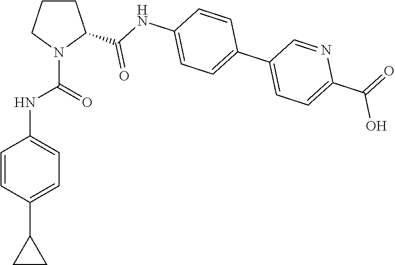 [SMILES: O=C(O)c1ccc(-c2ccc(NC(=O)[C@H]3CCCN3C(=O)Nc3ccc(C4CC4)cc3)cc2)cn1] | 5-[4-({1-[(4-cyclopropylphenyl)carbamoyl]- D-prolyl}amino)phenyl]pyridine-2- carboxylic acid |
| 123A | 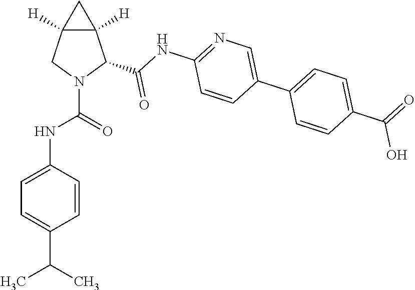 [SMILES: CC(C)c1ccc(NC(=O)N2C[C@H]3C[C@H]3[C@@H]2C(=O)Nc2ccc(-c3ccc(C(=O)O)cc3)cn2)cc1] | 4-(6-{[(1R,2R,5S)-3-{[4-(propan-2- yl)phenyl]carbamoyl}-3- azabicyclo[3.1.0]hexane-2- carbonyl]amino}pyridin-3-yl)benzoic acid |
| 124A | 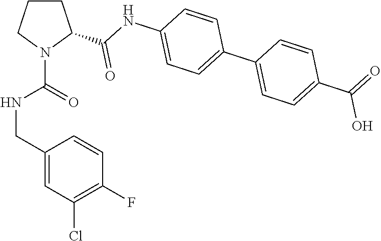 [SMILES: O=C(O)c1ccc(-c2ccc(NC(=O)[C@H]3CCCN3C(=O)NCc3ccc(F)c(Cl)c3)cc2)cc1] | 4′-[(1-{[(3-chloro-4- fluorophenyl)methyl]carbamoyl}-D- prolyl)amino][1,1′-biphenyl]-4-carboxylic acid |
| 125A | 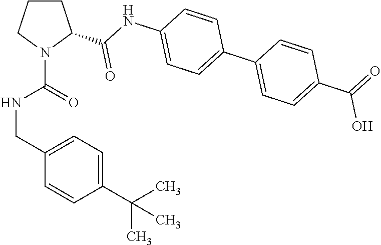 [SMILES: CC(C)(C)c1ccc(CNC(=O)N2CCC[C@@H]2C(=O)Nc2ccc(-c3ccc(C(=O)O)cc3)cc2)cc1] | 4′-[(1-{[(4-tert- butylphenyl)methyl]carbamoyl}-D- prolyl)amino][1,1′-biphenyl]-4-carboxylic acid |
| 126A | 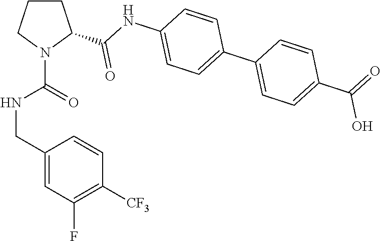 [SMILES: O=C(O)c1ccc(-c2ccc(NC(=O)[C@H]3CCCN3C(=O)NCc3ccc(C(F)(F)F)c(F)c3)cc2)cc1] | 4′-{[1-({[3-fluoro-4- (trifluoromethyl)phenyl]methyl}carbamoyl)- D-prolyl]amino}[1,1′-biphenyl]-4-carboxylic acid |
| 127A | 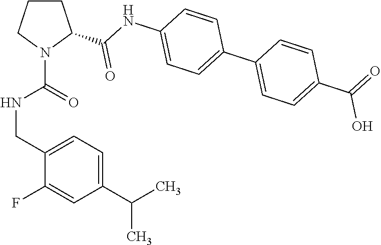 [SMILES: CC(C)c1ccc(CNC(=O)N2CCC[C@@H]2C(=O)Nc2ccc(-c3ccc(C(=O)O)cc3)cc2)c(F)c1] | 4′-{[1-({[2-fluoro-4-(propan-2- yl)phenyl]methyl}carbamoyl)-D- prolyl]amino}[1,1′-biphenyl]-4-carboxylic acid |
| 128A | 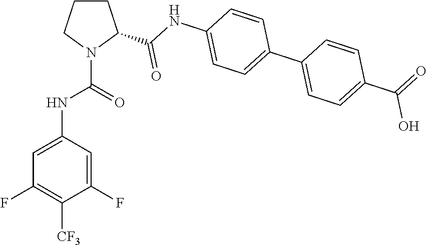 [SMILES: O=C(O)c1ccc(-c2ccc(NC(=O)[C@H]3CCCN3C(=O)Nc3cc(F)c(C(F)(F)F)c(F)c3)cc2)cc1] | 4′-[(1-{[3,5-difluoro-4- (trifluoromethyl)phenyl]carbamoyl}-D- prolyl)amino][1,1′-biphenyl]-4-carboxylic acid |
| 129A | 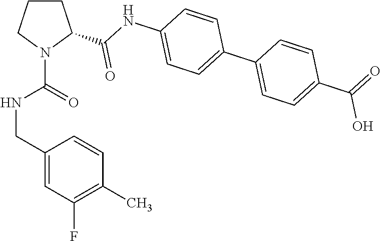 [SMILES: Cc1ccc(CNC(=O)N2CCC[C@@H]2C(=O)Nc2ccc(-c3ccc(C(=O)O)cc3)cc2)cc1F] | 4′-[(1-{[(3-fluoro-4- methylphenyl)methyl]carbamoyl}-D- prolyl)amino][1,1′-biphenyl]-4-carboxylic acid |
| 130A | 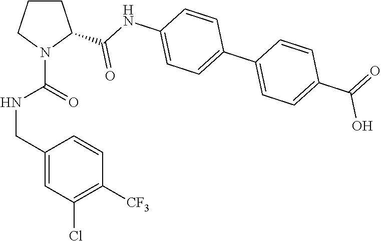 [SMILES: O=C(O)c1ccc(-c2ccc(NC(=O)[C@H]3CCCN3C(=O)NCc3ccc(C(F)(F)F)c(Cl)c3)cc2)cc1] | 4′-{[1-({[3-chloro-4- (trifluoromethyl)phenyl]methyl}carbamoyl)- D-prolyl]amino}[1,1′-biphenyl]-4-carboxylic acid |
| 131A | 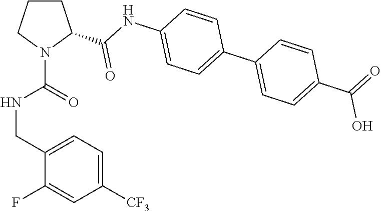 [SMILES: O=C(O)c1ccc(-c2ccc(NC(=O)[C@H]3CCCN3C(=O)NCc3ccc(C(F)(F)F)cc3F)cc2)cc1] | 4′-{[1-({[2-fluoro-4- (trifluoromethyl)phenyl]methyl}carbamoyl)- D-prolyl]amino}[1,1′-biphenyl]-4-carboxylic acid |
| 132A | 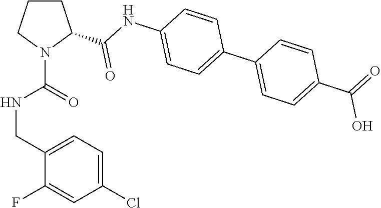 [SMILES: O=C(O)c1ccc(-c2ccc(NC(=O)[C@H]3CCCN3C(=O)NCc3ccc(Cl)cc3F)cc2)cc1] | 4′-[(1-{[(4-chloro-2- fluorophenyl)methyl]carbamoyl}-D- prolyl)amino][1,1′-biphenyl]-4-carboxylic acid |
| 133A | 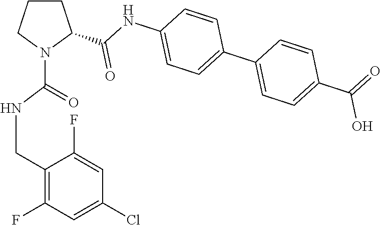 [SMILES: O=C(O)c1ccc(-c2ccc(NC(=O)[C@H]3CCCN3C(=O)NCc3c(F)cc(Cl)cc3F)cc2)cc1] | 4′-[(1-{[(4-chloro-2,6- difluorophenyl)methyl]carbamoyl}-D- prolyl)amino][1,1′-biphenyl]-4-carboxylic acid |
| 134A | 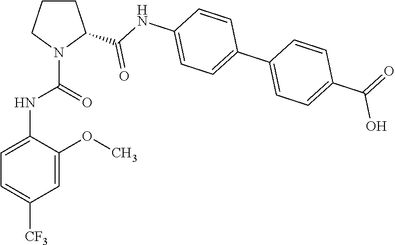 [SMILES: COc1cc(C(F)(F)F)ccc1NC(=O)N1CCC[C@@H]1C(=O)Nc1ccc(-c2ccc(C(=O)O)cc2)cc1] | 4′-[(1-{[2-methoxy-4- (trifluoromethyl)phenyl]carbamoyl}-D- prolyl)amino][1,1′-biphenyl]-4-carboxylic acid |
| 135A | 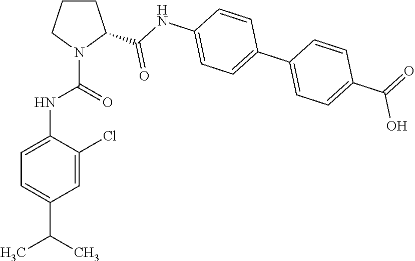 [SMILES: CC(C)c1ccc(NC(=O)N2CCC[C@@H]2C(=O)Nc2ccc(-c3ccc(C(=O)O)cc3)cc2)c(Cl)c1] | 4′-[(1-{[2-chloro-4-(propan-2- yl)phenyl]carbamoyl}-D-prolyl)amino][1,1′- biphenyl]-4-carboxylic acid |
| 136A | 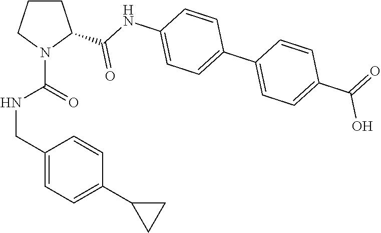 [SMILES: O=C(O)c1ccc(-c2ccc(NC(=O)[C@H]3CCCN3C(=O)NCc3ccc(C4CC4)cc3)cc2)cc1] | 4′-[(1-{[(4- cyclopropylphenyl)methyl]carbamoyl}-D- prolyl)amino][1,1′-biphenyl]-4-carboxylic acid |
| 137A | 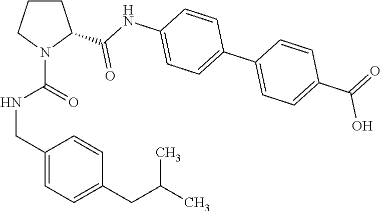 [SMILES: CC(C)Cc1ccc(CNC(=O)N2CCC[C@@H]2C(=O)Nc2ccc(-c3ccc(C(=O)O)cc3)cc2)cc1] | 4′-{[1-({[4-(2- methylpropyl)phenyl]methyl}carbamoyl)-D- prolyl]amino}[1,1′-biphenyl]-4-carboxylic acid |
| 138A | 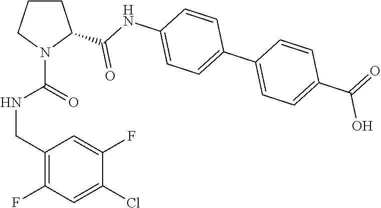 [SMILES: O=C(O)c1ccc(-c2ccc(NC(=O)[C@H]3CCCN3C(=O)NCc3cc(F)c(Cl)cc3F)cc2)cc1] | 4′-[(1-{[(4-chloro-2,5- difluorophenyl)methyl]carbamoyl}-D- prolyl)amino][1,1′-biphenyl]-4-carboxylic acid |
| 139A | 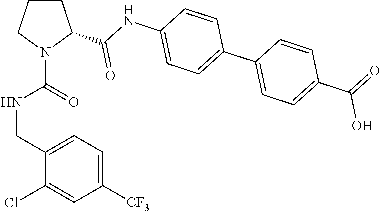 [SMILES: O=C(O)c1ccc(-c2ccc(NC(=O)[C@H]3CCCN3C(=O)NCc3ccc(C(F)(F)F)cc3Cl)cc2)cc1] | 4′-{[1-({[2-chloro-4- (trifluoromethyl)phenyl]methyl}carbamoyl)- D-prolyl]amino}[1,1′-biphenyl]-4-carboxylic acid |
| 140A | 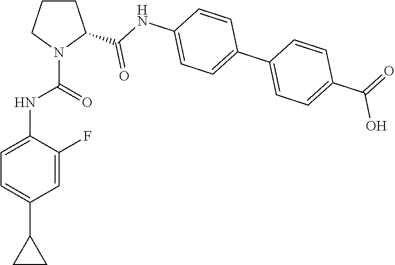 [SMILES: O=C(O)c1ccc(-c2ccc(NC(=O)[C@H]3CCCN3C(=O)Nc3ccc(C4CC4)cc3F)cc2)cc1] | 4′-({1-[(4-cyclopropyl-2- fluorophenyl)carbamoyl]-D- prolyl}amino)[1,1′-biphenyl]-4-carboxylic acid |
| 141A | 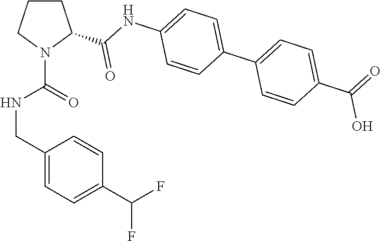 [SMILES: O=C(O)c1ccc(-c2ccc(NC(=O)[C@H]3CCCN3C(=O)NCc3ccc(C(F)F)cc3)cc2)cc1] | 4′-{[1-({[4- (difluoromethyl)phenyl]methyl}carbamoyl)- D-prolyl]amino}[1,1′-biphenyl]-4-carboxylic acid |
| 142A | 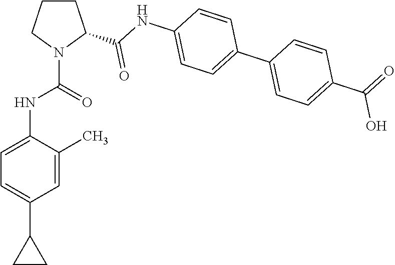 [SMILES: Cc1cc(C2CC2)ccc1NC(=O)N1CCC[C@@H]1C(=O)Nc1ccc(-c2ccc(C(=O)O)cc2)cc1] | 4′-({1-[(4-cyclopropyl-2- methylphenyl)carbamoyl]-D- prolyl}amino)[1,1′-biphenyl]-4-carboxylic acid |
| 143A | 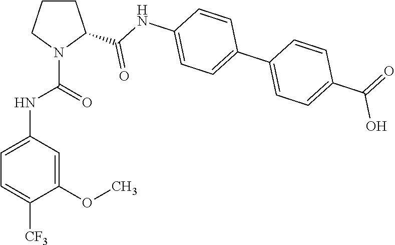 [SMILES: COc1cc(NC(=O)N2CCC[C@@H]2C(=O)Nc2ccc(-c3ccc(C(=O)O)cc3)cc2)ccc1C(F)(F)F] | 4′-[(1-{[3-methoxy-4- (trifluoromethyl)phenyl]carbamoyl}-D- prolyl)amino][1,1′-biphenyl]-4-carboxylic acid |
| 144A | 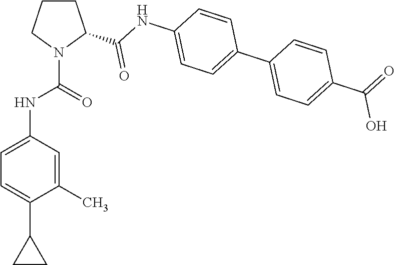 [SMILES: Cc1cc(NC(=O)N2CCC[C@@H]2C(=O)Nc2ccc(-c3ccc(C(=O)O)cc3)cc2)ccc1C1CC1] | 4′-({1-[(4-cyclopropyl-3- methylphenyl)carbamoyl]-D- prolyl}amino)[1,1′-biphenyl]-4-carboxylic acid |
| 145A | 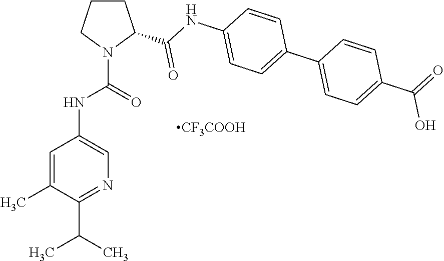 [SMILES: Cc1cc(NC(=O)N2CCC[C@@H]2C(=O)Nc2ccc(-c3ccc(C(=O)O)cc3)cc2)cnc1C(C)C ; "·CF3COOH"] | 4′-[(1-{[5-methyl-6-(propan-2-yl)pyridin-3- yl]carbamoyl}-D-prolyl)amino][1,1′- biphenyl]-4-carboxylic acid, trifluoroacetate salt |
| 146A | 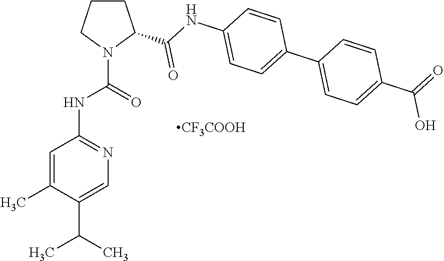 [SMILES: Cc1cc(NC(=O)N2CCC[C@@H]2C(=O)Nc2ccc(-c3ccc(C(=O)O)cc3)cc2)ncc1C(C)C ; "·CF3COOH"] | 4′-[(1-{[4-methyl-5-(propan-2-yl)pyridin-2- yl]carbamoyl}-D-prolyl)amino][1,1′- biphenyl]-4-carboxylic acid, trifluoroacetate salt |
| 147A | 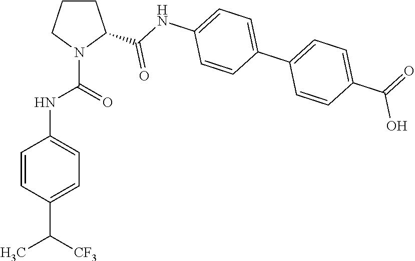 [SMILES: CC(c1ccc(NC(=O)N2CCC[C@@H]2C(=O)Nc2ccc(-c3ccc(C(=O)O)cc3)cc2)cc1)C(F)(F)F] | 4′-[(1-{[4-(1,1,1-trifluoropropan-2- yl)phenyl]carbamoyl}-D-prolyl)amino][1,1′- biphenyl]-4-carboxylic acid |
| 148A | 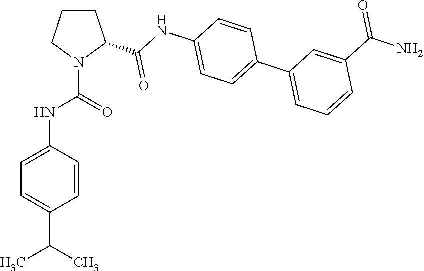 [SMILES: CC(C)c1ccc(NC(=O)N2CCC[C@@H]2C(=O)Nc2ccc(-c3cccc(C(N)=O)c3)cc2)cc1] | (2R)-N²-(3′-carbamoyl[1,1′-biphenyl]-4-yl)- N¹-[4-(propan-2-yl)phenyl]pyrrolidine-1,2- dicarboxamide |
| 149A | 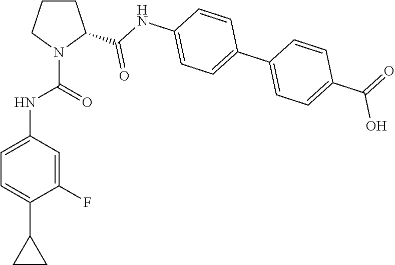 [SMILES: O=C(O)c1ccc(-c2ccc(NC(=O)[C@H]3CCCN3C(=O)Nc3ccc(C4CC4)c(F)c3)cc2)cc1] | 4′-({1-[(4-cyclopropyl-3- fluorophenyl)carbamoyl]-D- prolyl}amino)[1,1′-biphenyl]-4-carboxylic acid |
| 150A | 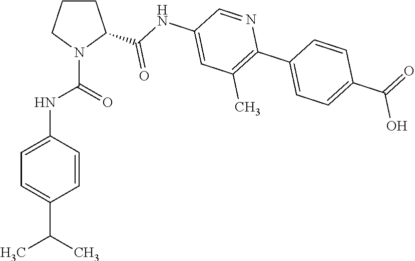 [SMILES: Cc1cc(NC(=O)[C@H]2CCCN2C(=O)Nc2ccc(C(C)C)cc2)cnc1-c1ccc(C(=O)O)cc1] | 4-{3-methyl-5-[(1-{[4-(propan-2- yl)phenyl]carbamoyl}-D- prolyl)amino]pyridin-2-yl}benzoic acid |
| 151A | 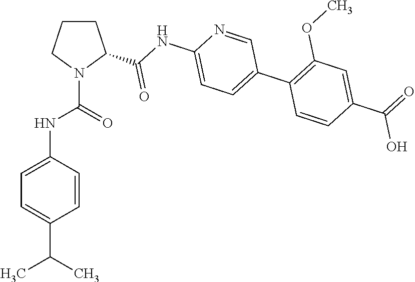 [SMILES: COc1cc(C(=O)O)ccc1-c1ccc(NC(=O)[C@H]2CCCN2C(=O)Nc2ccc(C(C)C)cc2)nc1] | 3-methoxy-4-{6-[(1-{[4-(propan-2- yl)phenyl]carbamoyl}-D- prolyl)amino]pyridin-3-yl}benzoic acid |
| 152A | 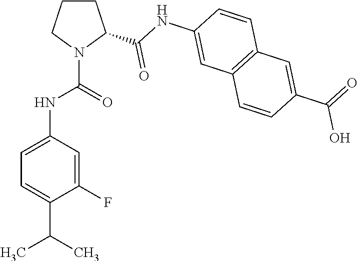 [SMILES: CC(C)c1ccc(NC(=O)N2CCC[C@@H]2C(=O)Nc2ccc3cc(C(=O)O)ccc3c2)cc1F] | 6-[(1-{[3-fluoro-4-(propan-2- yl)phenyl]carbamoyl}-D- prolyl)amino]naphthalene-2-carboxylic acid |
| 153A | 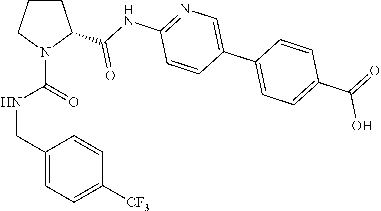 [SMILES: O=C(O)c1ccc(-c2ccc(NC(=O)[C@H]3CCCN3C(=O)NCc3ccc(C(F)(F)F)cc3)nc2)cc1] | 4-(6-{[1-({[4- (trifluoromethyl)phenyl]methyl}carbamoyl)- D-prolyl]amino}pyridin-3-yl)benzoic acid |
| 154A | 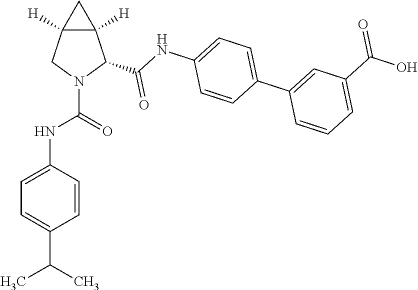 [SMILES: CC(C)c1ccc(NC(=O)N2C[C@H]3C[C@H]3[C@@H]2C(=O)Nc2ccc(-c3cccc(C(=O)O)c3)cc2)cc1] | 4′-{[(1R,2R,5S)-3-{[4-(propan-2- yl)phenyl]carbamoyl}-3- azabicyclo[3.1.0]hexane-2- carbonyl]amino}[1,1′-biphenyl]-3-carboxylic acid |
| 155A | 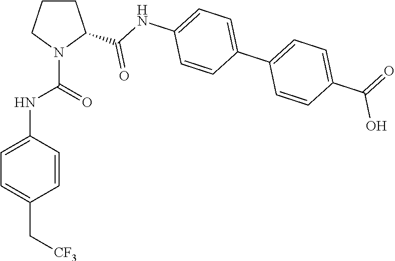 [SMILES: O=C(O)c1ccc(-c2ccc(NC(=O)[C@H]3CCCN3C(=O)Nc3ccc(CC(F)(F)F)cc3)cc2)cc1] | 4′-[(1-{[4-(2,2,2- trifluoroethyl)phenyl]carbamoyl}-D- prolyl)amino][1,1′-biphenyl]-4-carboxylic acid |
| 156A | 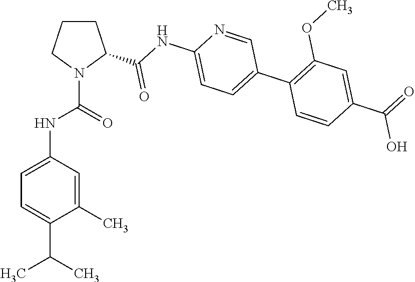 [SMILES: COc1cc(C(=O)O)ccc1-c1ccc(NC(=O)[C@H]2CCCN2C(=O)Nc2ccc(C(C)C)c(C)c2)nc1] | 3-methoxy-4-{6-[(1-{[3-methyl-4-(propan-2- yl)phenyl]carbamoyl}-D- prolyl)amino]pyridin-3-yl}benzoic acid |
| 157A | 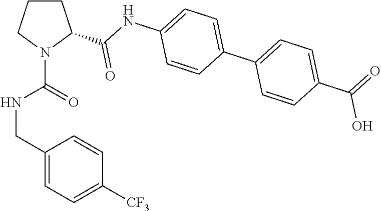 [SMILES: O=C(O)c1ccc(-c2ccc(NC(=O)[C@H]3CCCN3C(=O)NCc3ccc(C(F)(F)F)cc3)cc2)cc1] | 4′-{[1-({[4- (trifluoromethyl)phenyl]methyl}carbamoyl)- D-prolyl]amino}[1,1′-biphenyl]-4-carboxylic acid |
| 158A | 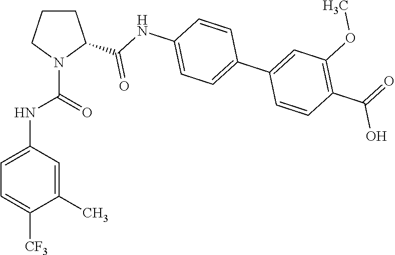 [SMILES: COc1cc(-c2ccc(NC(=O)[C@H]3CCCN3C(=O)Nc3ccc(C(F)(F)F)c(C)c3)cc2)ccc1C(=O)O] | 3-methoxy-4′-[(1-{[3-methyl-4- (trifluoromethyl)phenyl]carbamoyl}-D- prolyl)amino][1,1′-biphenyl]-4-carboxylic acid |
| 159A | 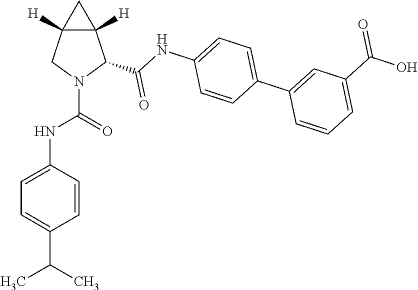 [SMILES: CC(C)c1ccc(NC(=O)N2C[C@@H]3C[C@@H]3[C@@H]2C(=O)Nc2ccc(-c3cccc(C(=O)O)c3)cc2)cc1] | 4′-{[(1S,2R,5R)-3-{[4-(propan-2- yl)phenyl]carbamoyl}-3- azabicyclo[3.1.0]hexane-2- carbonyl]amino}[1,1′-biphenyl]-3-carboxylic acid |
| 160A | 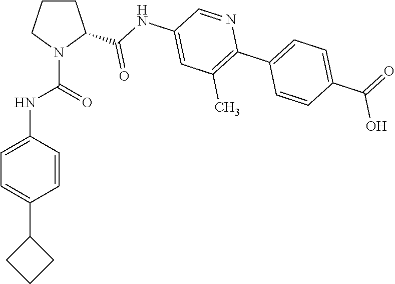 [SMILES: Cc1cc(NC(=O)[C@H]2CCCN2C(=O)Nc2ccc(C3CCC3)cc2)cnc1-c1ccc(C(=O)O)cc1] | 4-[5-({1-[(4-cyclobutylphenyl)carbamoyl]-D- prolyl}amino)-3-methylpyridin-2-yl]benzoic acid |
| 161A | 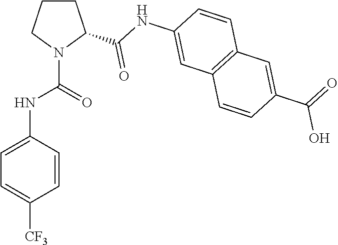 [SMILES: O=C(O)c1ccc2cc(NC(=O)[C@H]3CCCN3C(=O)Nc3ccc(C(F)(F)F)cc3)ccc2c1] | 6-[(1-{[4- (trifluoromethyl)phenyl]carbamoyl}-D- prolyl)amino]naphthalene-2-carboxylic acid |
| 162A | 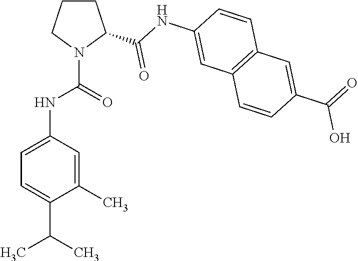 [SMILES: Cc1cc(NC(=O)N2CCC[C@@H]2C(=O)Nc2ccc3cc(C(=O)O)ccc3c2)ccc1C(C)C] | 6-[(1-{[3-methyl-4-(propan-2- yl)phenyl]carbamoyl}-D- prolyl)amino]naphthalene-2-carboxylic acid |
| 163A | 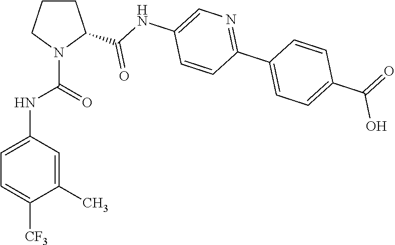 [SMILES: Cc1cc(NC(=O)N2CCC[C@@H]2C(=O)Nc2ccc(-c3ccc(C(=O)O)cc3)nc2)ccc1C(F)(F)F] | 4-{5-[(1-{[3-methyl-4- (trifluoromethyl)phenyl]carbamoyl}-D- prolyl)amino]pyridin-2-yl}benzoic acid |
| 164A | 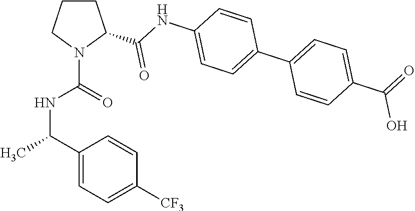 [SMILES: C[C@H](NC(=O)N1CCC[C@@H]1C(=O)Nc1ccc(-c2ccc(C(=O)O)cc2)cc1)c1ccc(C(F)(F)F)cc1] | 4′-{[1-({(1S)-1-[4- (trifluoromethyl)phenyl]ethyl}carbamoyl)-D- prolyl]amino}[1,1′-biphenyl]-4-carboxylic acid |
| 165A | 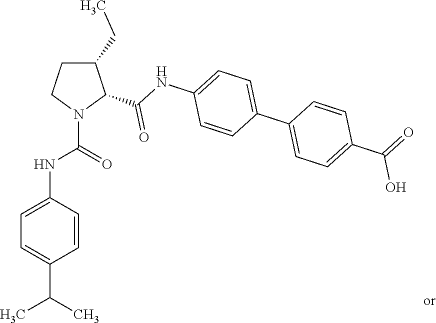 [SMILES: CC[C@H]1CCN(C(=O)Nc2ccc(C(C)C)cc2)[C@H]1C(=O)Nc1ccc(-c2ccc(C(=O)O)cc2)cc1] | 4′-{[(3R)-3-ethyl-1-{[4-(propan-2- yl)phenyl]carbamoyl}-L-prolyl]amino}[1,1′- biphenyl]-4-carboxylic acid or 4′-{[(3S)-3-ethyl-1-{[4-(propan-2- yl)phenyl]carbamoyl}-D-prolyl]amino}[1,1′- biphenyl]-4-carboxylic acid |
| | 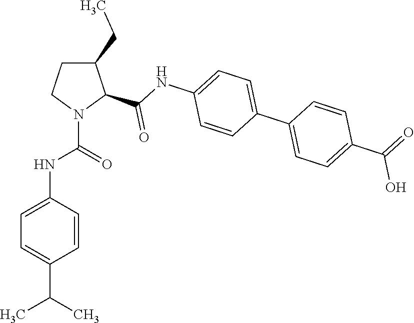 [SMILES: CC[C@@H]1CCN(C(=O)Nc2ccc(C(C)C)cc2)[C@@H]1C(=O)Nc1ccc(-c2ccc(C(=O)O)cc2)cc1] | |
| 166A | 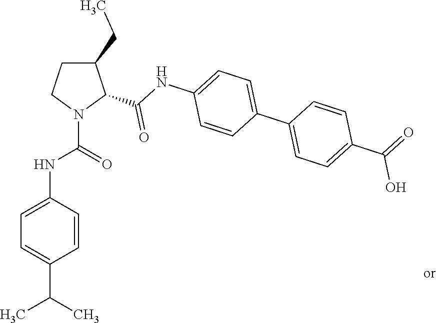 [SMILES: CC[C@@H]1CCN(C(=O)Nc2ccc(C(C)C)cc2)[C@H]1C(=O)Nc1ccc(-c2ccc(C(=O)O)cc2)cc1] | 4′-{[(3R)-3-ethyl-1-{[4-(propan-2- yl)phenyl]carbamoyl}-D-prolyl]amino}[1,1′- biphenyl]-4-carboxylic acid or 4′-{[(3S)-3-ethyl-1-{[4-(propan-2- yl)phenyl]carbamoyl}-L-prolyl]amino}[1,1′- biphenyl]-4-carboxylic acid |
| | 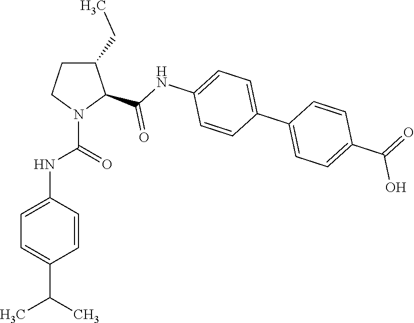 [SMILES: CC[C@H]1CCN(C(=O)Nc2ccc(C(C)C)cc2)[C@@H]1C(=O)Nc1ccc(-c2ccc(C(=O)O)cc2)cc1] | |
| 167A | 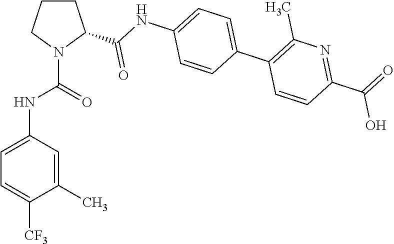 [SMILES: Cc1cc(NC(=O)N2CCC[C@@H]2C(=O)Nc2ccc(-c3ccc(C(=O)O)nc3C)cc2)ccc1C(F)(F)F] | 6-methyl-5-{4-[(1-{[3-methyl-4- (trifluoromethyl)phenyl]carbamoyl}-D- prolyl)amino]phenyl}pyridine-2-carboxylic acid |
| 168A | 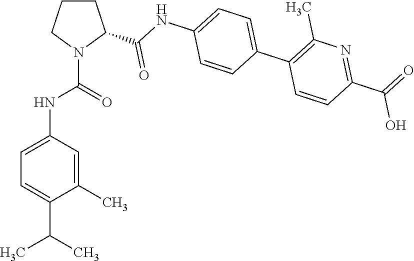 [SMILES: Cc1cc(NC(=O)N2CCC[C@@H]2C(=O)Nc2ccc(-c3ccc(C(=O)O)nc3C)cc2)ccc1C(C)C] | 6-methyl-5-{4-[(1-{[3-methyl-4-(propan-2- yl)phenyl]carbamoyl}-D- prolyl)amino]phenyl}pyridine-2-carboxylic acid |
| 169A | 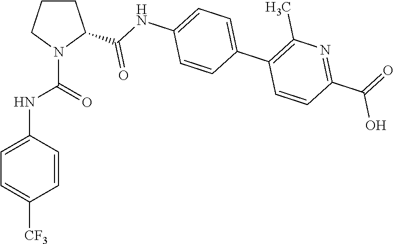 [SMILES: Cc1nc(C(=O)O)ccc1-c1ccc(NC(=O)[C@H]2CCCN2C(=O)Nc2ccc(C(F)(F)F)cc2)cc1] | 6-methyl-5-{4-[(1-{[4- (trifluoromethyl)phenyl]carbamoyl}-D- prolyl)amino]phenyl}pyridine-2-carboxylic acid |
| 170A | 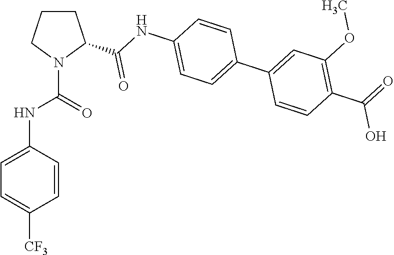 [SMILES: COc1cc(-c2ccc(NC(=O)[C@H]3CCCN3C(=O)Nc3ccc(C(F)(F)F)cc3)cc2)ccc1C(=O)O] | 3-methoxy-4′-[(1-{[4- (trifluoromethyl)phenyl]carbamoyl}-D- prolyl)amino][1,1′-biphenyl]-4-carboxylic acid |
| 171A | 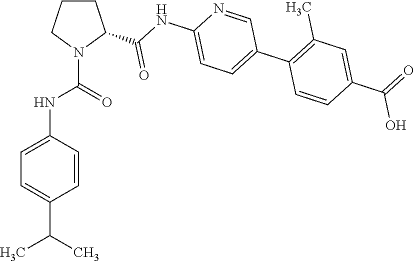 [SMILES: Cc1cc(C(=O)O)ccc1-c1ccc(NC(=O)[C@H]2CCCN2C(=O)Nc2ccc(C(C)C)cc2)nc1] | 3-methyl-4-{6-[(1-{[4-(propan-2- yl)phenyl]carbamoyl}-D- prolyl)amino]pyridin-3-yl}benzoic acid |
| 172A | 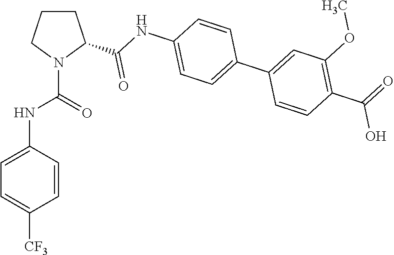 [SMILES: COc1cc(-c2ccc(NC(=O)[C@H]3CCCN3C(=O)Nc3ccc(C(F)(F)F)cc3)cc2)ccc1C(=O)O] | 3-methyl-4-{6-[(1-{[4- (trifluoromethyl)phenyl]carbamoyl}-D- prolyl)amino]pyridin-3-yl}benzoic acid |
| 173A | 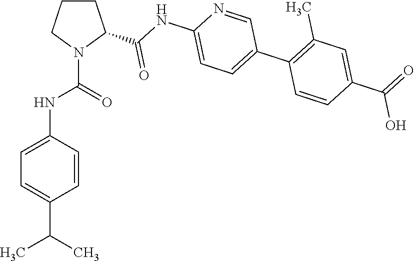 [SMILES: Cc1cc(C(=O)O)ccc1-c1ccc(NC(=O)[C@H]2CCCN2C(=O)Nc2ccc(C(C)C)cc2)nc1] | 3-methyl-4-{6-[(1-{[3-methyl-4-(propan-2- yl)phenyl]carbamoyl}-D- prolyl)amino]pyridin-3-yl}benzoic acid |
| 174A | 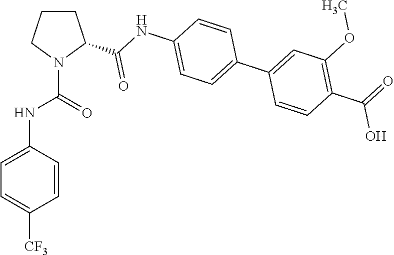 [SMILES: COc1cc(-c2ccc(NC(=O)[C@H]3CCCN3C(=O)Nc3ccc(C(F)(F)F)cc3)cc2)ccc1C(=O)O] | 5-[4-({1-[(4-cyclopropyl-3- methylphenyl)carbamoyl]-D- prolyl}amino)phenyl]pyridine-2-carboxylic acid |
| 175A | 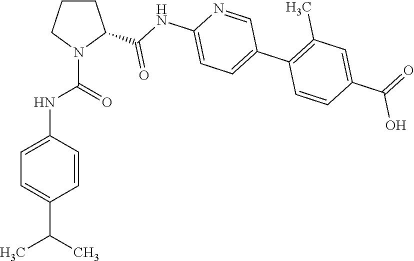 [SMILES: Cc1cc(C(=O)O)ccc1-c1ccc(NC(=O)[C@H]2CCCN2C(=O)Nc2ccc(C(C)C)cc2)nc1] | 3-{6-[(1-{[3-methyl-4- (trifluoromethyl)phenyl]carbamoyl}-D- prolyl)amino]pyridin-3-yl}benzoic acid |
| 176A | 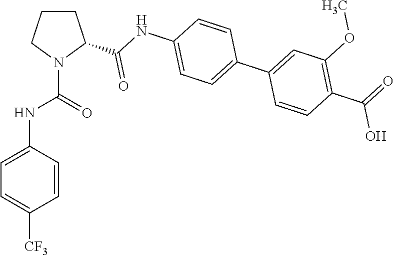 [SMILES: COc1cc(-c2ccc(NC(=O)[C@H]3CCCN3C(=O)Nc3ccc(C(F)(F)F)cc3)cc2)ccc1C(=O)O] | 4-{6-[(1-{[3-fluoro-4-(propan-2- yl)phenyl]carbamoyl}-D- prolyl)amino]pyridin-3-yl}-3-methylbenzoic acid |
| 177A | 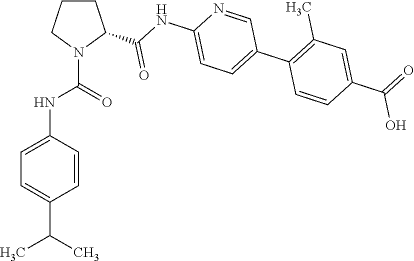 [SMILES: Cc1cc(C(=O)O)ccc1-c1ccc(NC(=O)[C@H]2CCCN2C(=O)Nc2ccc(C(C)C)cc2)nc1] | 4-{6-[(1-{[3-methyl-4- (trifluoromethyl)phenyl]carbamoyl}-D- prolyl)amino]pyridin-3-yl}benzoic acid |
| 178A | 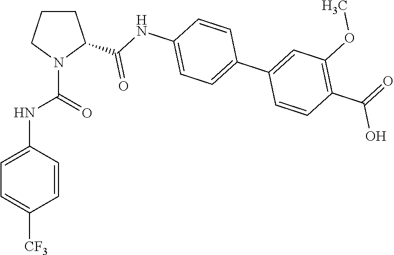 [SMILES: COc1cc(-c2ccc(NC(=O)[C@H]3CCCN3C(=O)Nc3ccc(C(F)(F)F)cc3)cc2)ccc1C(=O)O] | 4-(6-{[1-({4-[1- (trifluoromethyl)cyclopropyl]phenyl} carbamoyl)-D-prolyl]amino}pyridin-3-yl) benzoic acid |
| 179A | 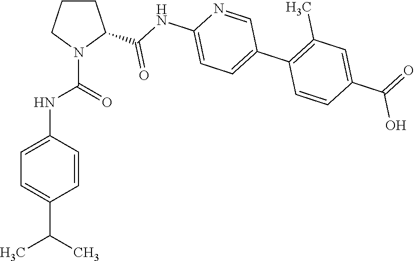 [SMILES: Cc1cc(C(=O)O)ccc1-c1ccc(NC(=O)[C@H]2CCCN2C(=O)Nc2ccc(C(C)C)cc2)nc1] | 4-[6-({1-[(4-cyclopropyl-3- methylphenyl)carbamoyl]-D- prolyl}amino)pyridin-3-yl]benzoic acid |
| 180A | 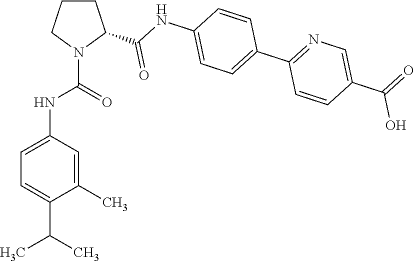 [SMILES: Cc1cc(NC(=O)N2CCC[C@@H]2C(=O)Nc2ccc(-c3ccc(C(=O)O)cn3)cc2)ccc1C(C)C] | 6-{4-[(1-{[3-methyl-4-(propan-2- yl)phenyl]carbamoyl}-D- prolyl)amino]phenyl}pyridine-3-carboxylic acid |
| 181A | 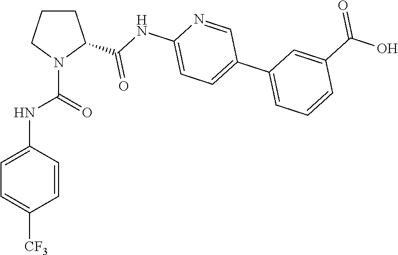 [SMILES: O=C(O)c1cccc(-c2ccc(NC(=O)[C@H]3CCCN3C(=O)Nc3ccc(C(F)(F)F)cc3)nc2)c1] | 3-{6-[(1-{[4- (trifluoromethyl)phenyl]carbamoyl}-D- prolyl)amino]pyridin-3-yl}benzoic acid |
| 182A | 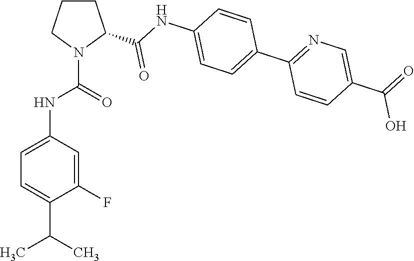 [SMILES: CC(C)c1ccc(NC(=O)N2CCC[C@@H]2C(=O)Nc2ccc(-c3ccc(C(=O)O)cn3)cc2)cc1F] | 6-{4-[(1-{[3-fluoro-4-(propan-2- yl)phenyl]carbamoyl}-D- prolyl)amino]phenyl}pyridine-3-carboxylic acid |
| 183A | 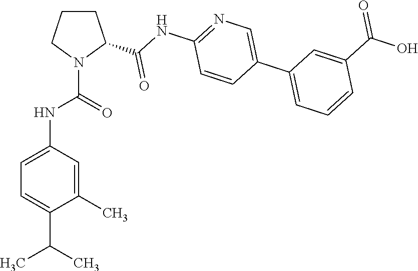 [SMILES: Cc1cc(NC(=O)N2CCC[C@@H]2C(=O)Nc2ccc(-c3cccc(C(=O)O)c3)cn2)ccc1C(C)C] | 3-{6-[(1-{[3-methyl-4-(propan-2- yl)phenyl]carbamoyl}-D- prolyl)amino]pyridin-3-yl}benzoic acid |
| 184A | 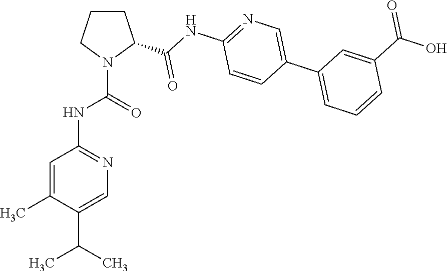 [SMILES: Cc1cc(NC(=O)N2CCC[C@@H]2C(=O)Nc2ccc(-c3cccc(C(=O)O)c3)cn2)ncc1C(C)C] | 3-{6-[(1-{[4-methyl-5-(propan-2-yl)pyridin- 2-yl]carbamoyl}-D-prolyl)amino]pyridin-3- yl}benzoic acid |
| 185A | 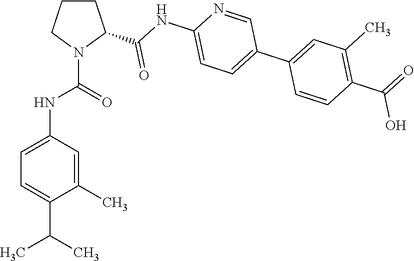 [SMILES: Cc1cc(-c2ccc(NC(=O)[C@H]3CCCN3C(=O)Nc3ccc(C(C)C)c(C)c3)nc2)ccc1C(=O)O] | 2-methyl-4-{6-[(1-{[3-methyl-4-(propan-2- yl)phenyl]carbamoyl}-D- prolyl)amino]pyridin-3-yl}benzoic acid |
| 186A | 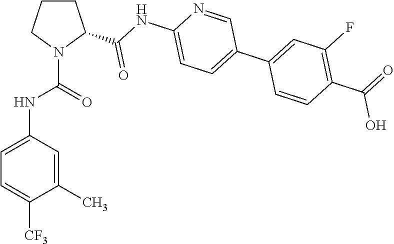 [SMILES: Cc1cc(NC(=O)N2CCC[C@@H]2C(=O)Nc2ccc(-c3ccc(C(=O)O)c(F)c3)cn2)ccc1C(F)(F)F] | 2-fluoro-4-{6-[(1-{[3-methyl-4- (trifluoromethyl)phenyl]carbamoyl}-D- prolyl)amino]pyridin-3-yl}benzoic acid |
| 187A | 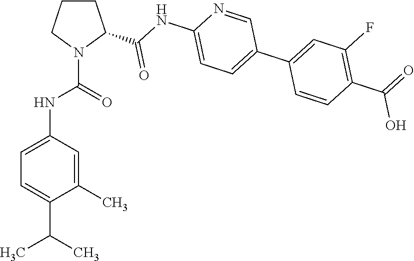 [SMILES: Cc1cc(NC(=O)N2CCC[C@@H]2C(=O)Nc2ccc(-c3ccc(C(=O)O)c(F)c3)cn2)ccc1C(C)C] | 2-fluoro-4-{6-[(1-{[3-methyl-4-(propan-2- yl)phenyl]carbamoyl}-D- prolyl)amino]pyridin-3-yl}benzoic acid |
| 188A | 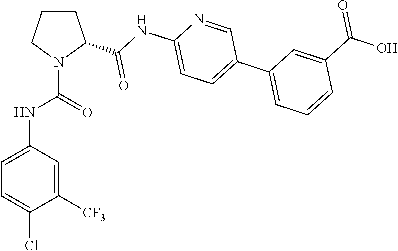 [SMILES: O=C(O)c1cccc(-c2ccc(NC(=O)[C@H]3CCCN3C(=O)Nc3ccc(Cl)c(C(F)(F)F)c3)nc2)c1] | 3-{6-[(1-{[4-chloro-3- (trifluoromethyl)phenyl]carbamoyl}-D- prolyl)amino]pyridin-3-yl}benzoic acid |
| 189A | 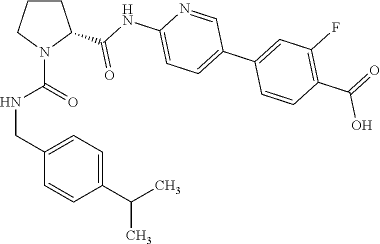 [SMILES: CC(C)c1ccc(CNC(=O)N2CCC[C@@H]2C(=O)Nc2ccc(-c3ccc(C(=O)O)c(F)c3)cn2)cc1] | 2-fluoro-4-(6-{[1-({[4-(propan-2- yl)phenyl]methyl}carbamoyl)-D- prolyl]amino}pyridin-3-yl)benzoic acid |
| 190A | 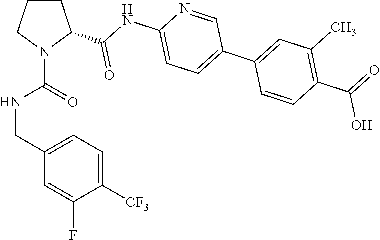 [SMILES: Cc1cc(-c2ccc(NC(=O)[C@H]3CCCN3C(=O)NCc3ccc(C(F)(F)F)c(F)c3)nc2)ccc1C(=O)O] | 4-(6-{[1-({[3-fluoro-4- (trifluoromethyl)phenyl]methyl}carbamoyl)- D-prolyl]amino}pyridin-3-yl)-2- methylbenzoic acid |
| 191A | 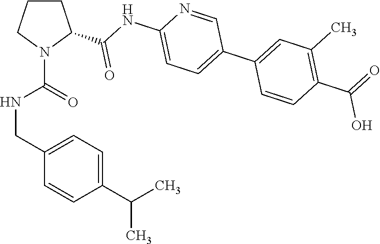 [SMILES: Cc1cc(-c2ccc(NC(=O)[C@H]3CCCN3C(=O)NCc3ccc(C(C)C)cc3)nc2)ccc1C(=O)O] | 2-methyl-4-(6-{[1-({[4-(propan-2- yl)phenyl]methyl}carbamoyl)-D- prolyl]amino}pyridin-3-yl)benzoic acid |
| 192A | 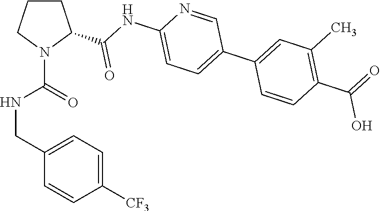 [SMILES: Cc1cc(-c2ccc(NC(=O)[C@H]3CCCN3C(=O)NCc3ccc(C(F)(F)F)cc3)nc2)ccc1C(=O)O] | 2-methyl-4-(6-{[1-({[4- (trifluoromethyl)phenyl]methyl}carbamoyl)- D-prolyl]amino}pyridin-3-yl)benzoic acid |
| 193A | 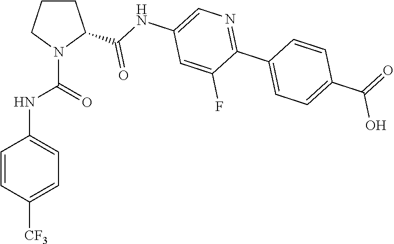 [SMILES: O=C(O)c1ccc(-c2ncc(NC(=O)[C@H]3CCCN3C(=O)Nc3ccc(C(F)(F)F)cc3)cc2F)cc1] | 4-{3-fluoro-5-[(1-{[4- (trifluoromethyl)phenyl]carbamoyl}-D- prolyl)amino]pyridin-2-yl}benzoic acid |
| 194A | 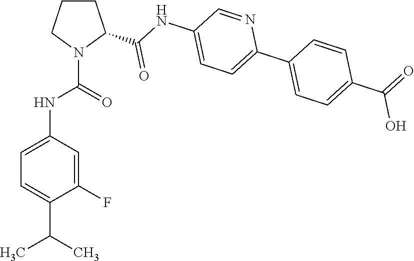 [SMILES: CC(C)c1ccc(NC(=O)N2CCC[C@@H]2C(=O)Nc2ccc(-c3ccc(C(=O)O)cc3)nc2)cc1F] | 4-{5-[(1-{[3-fluoro-4-(propan-2- yl)phenyl]carbamoyl}-D- prolyl)amino]pyridin-2-yl}benzoic acid |
| 195A | 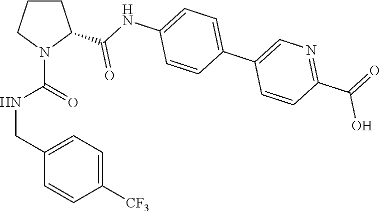 [SMILES: O=C(O)c1ccc(-c2ccc(NC(=O)[C@H]3CCCN3C(=O)NCc3ccc(C(F)(F)F)cc3)cc2)cn1] | 5-(4-{[1-({[4- (trifluoromethyl)phenyl]methyl}carbamoyl)- D-prolyl]amino}phenyl)pyridine-2- carboxylic acid |
| 196A | 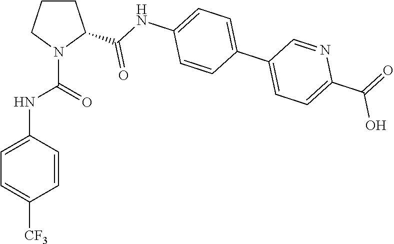 [SMILES: O=C(O)c1ccc(-c2ccc(NC(=O)[C@H]3CCCN3C(=O)Nc3ccc(C(F)(F)F)cc3)cc2)cn1] | 5-{4-[(1-{[4- (trifluoromethyl)phenyl]carbamoyl}-D- prolyl)amino]phenyl}pyridine-2-carboxylic acid |
| 197A | 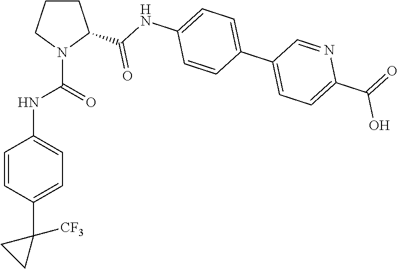 [SMILES: O=C(O)c1ccc(-c2ccc(NC(=O)[C@H]3CCCN3C(=O)Nc3ccc(C4(C(F)(F)F)CC4)cc3)cc2)cn1] | 5-(4-{[1-({4-[1- (trifluoromethyl)cyclopropyl]phenyl} carbamoyl)-D-prolyl]amino}phenyl) pyridine-2-carboxylic acid |
| 198A | 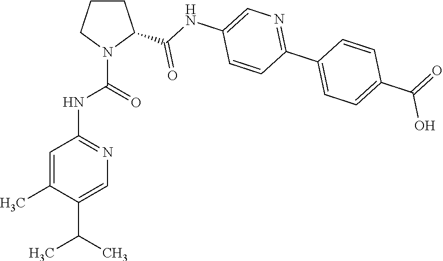 [SMILES: Cc1cc(NC(=O)N2CCC[C@@H]2C(=O)Nc2ccc(-c3ccc(C(=O)O)cc3)nc2)ncc1C(C)C] | 4-{5-[(1-{[4-methyl-5-(propan-2-yl)pyridin- 2-yl]carbamoyl}-D-prolyl)amino]pyridin-2- yl}benzoic acid |
| 199A | 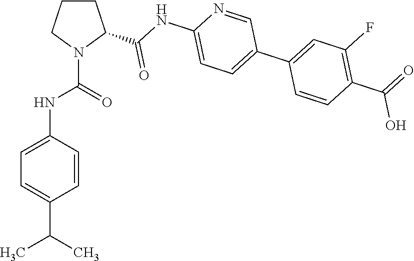 [SMILES: CC(C)c1ccc(NC(=O)N2CCC[C@@H]2C(=O)Nc2ccc(-c3ccc(C(=O)O)c(F)c3)cn2)cc1] | 2-fluoro-4-{6-[(1-{[4-(propan-2- yl)phenyl]carbamoyl}-D- prolyl)amino]pyridin-3-yl}benzoic acid |
| 200A | 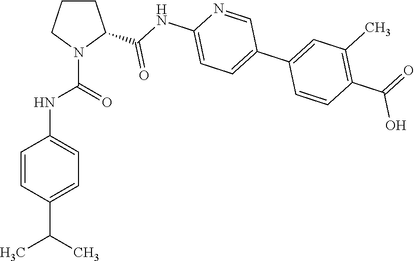 [SMILES: Cc1cc(-c2ccc(NC(=O)[C@H]3CCCN3C(=O)Nc3ccc(C(C)C)cc3)nc2)ccc1C(=O)O] | 2-methyl-4-{6-[(1-{[4-(propan-2- yl)phenyl]carbamoyl}-D- prolyl)amino]pyridin-3-yl}benzoic acid |
| 201A | 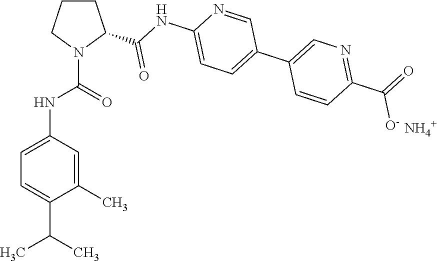 [SMILES: Cc1cc(NC(=O)N2CCC[C@@H]2C(=O)Nc2ccc(-c3ccc(C(=O)[O-])nc3)cn2)ccc1C(C)C ; [NH4+]] | ammonium 6′-[(1-{[3-methyl-4-(propan-2- yl)phenyl]carbamoyl}-D-prolyl)amino][3,3′- bipyridine]-6-carboxylate |
| 202A | 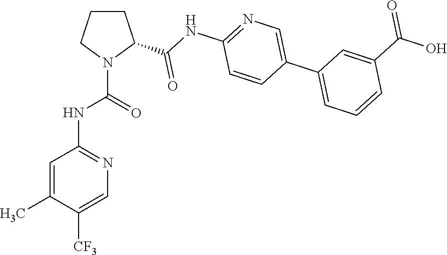 [SMILES: Cc1cc(NC(=O)N2CCC[C@@H]2C(=O)Nc2ccc(-c3cccc(C(=O)O)c3)cn2)ncc1C(F)(F)F] | 3-{6-[(1-{[4-methyl-5- (trifluoromethyl)pyridin-2-yl]carbamoyl}-D- prolyl)amino]pyridin-3-yl}benzoic acid |
| 203A | 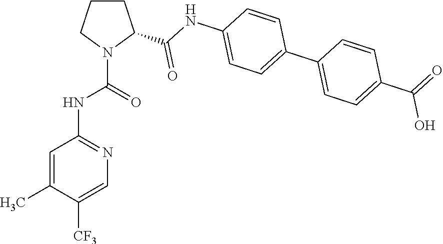 [SMILES: Cc1cc(NC(=O)N2CCC[C@@H]2C(=O)Nc2ccc(-c3ccc(C(=O)O)cc3)cc2)ncc1C(F)(F)F] | 4′-[(1-{[4-methyl-5-(trifluoromethyl)pyridin- 2-yl]carbamoyl}-D-prolyl)amino][1,1′- biphenyl]-4-carboxylic acid |
| 204A | 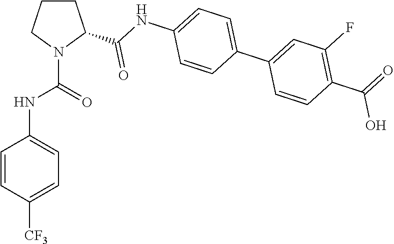 [SMILES: O=C(O)c1ccc(-c2ccc(NC(=O)[C@H]3CCCN3C(=O)Nc3ccc(C(F)(F)F)cc3)cc2)cc1F] | 3-fluoro-4′-[(1-{[4- (trifluoromethyl)phenyl]carbamoyl}-D- prolyl)amino][1,1′-biphenyl]-4-carboxylic acid |
| 205A | 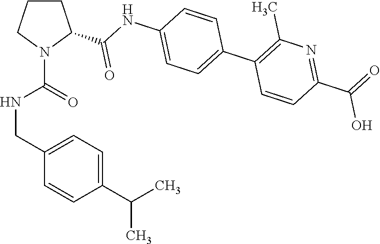 [SMILES: Cc1nc(C(=O)O)ccc1-c1ccc(NC(=O)[C@H]2CCCN2C(=O)NCc2ccc(C(C)C)cc2)cc1] | 6-methyl-5-(4-{[1-({[4-(propan-2- yl)phenyl]methyl}carbamoyl)-D- prolyl]amino}phenyl)pyridine-2-carboxylic acid |
| 206A | 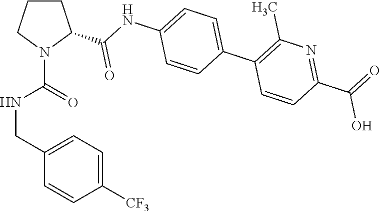 [SMILES: Cc1nc(C(=O)O)ccc1-c1ccc(NC(=O)[C@H]2CCCN2C(=O)NCc2ccc(C(F)(F)F)cc2)cc1] | 6-methyl-5-(4-{[1-({[4- (trifluoromethyl)phenyl]methyl}carbamoyl)- D-prolyl]amino}phenyl)pyridine-2- carboxylic acid |
| 207A | 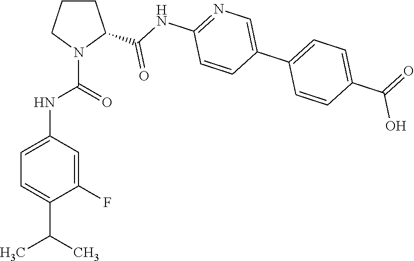 [SMILES: CC(C)c1ccc(NC(=O)N2CCC[C@@H]2C(=O)Nc2ccc(-c3ccc(C(=O)O)cc3)cn2)cc1F] | 4-{6-[(1-{[3-fluoro-4-(propan-2- yl)phenyl]carbamoyl}-D- prolyl)amino]pyridin-3-yl}benzoic acid |
| 208A | 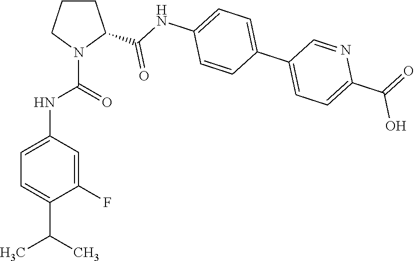 [SMILES: CC(C)c1ccc(NC(=O)N2CCC[C@@H]2C(=O)Nc2ccc(-c3ccc(C(=O)O)nc3)cc2)cc1F] | 5-{4-[(1-{[3-fluoro-4-(propan-2- yl)phenyl]carbamoyl}-D- prolyl)amino]phenyl}pyridine-2-carboxylic acid |
| 209A | 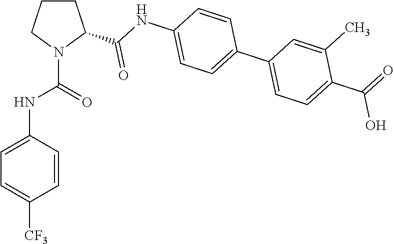 [SMILES: Cc1cc(-c2ccc(NC(=O)[C@H]3CCCN3C(=O)Nc3ccc(C(F)(F)F)cc3)cc2)ccc1C(=O)O] | 3-methyl-4′-[(1-{[4- (trifluoromethyl)phenyl]carbamoyl}-D- prolyl)amino][1,1′-biphenyl]-4-carboxylic acid |
| 210A | 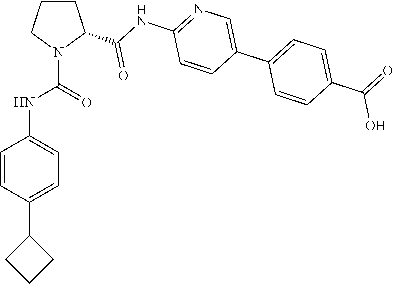 [SMILES: O=C(O)c1ccc(-c2ccc(NC(=O)[C@H]3CCCN3C(=O)Nc3ccc(C4CCC4)cc3)nc2)cc1] | 4-[6-({1-[(4-cyclobutylphenyl)carbamoyl]-D- prolyl}amino)pyridin-3-yl]benzoic acid |
| 211A | 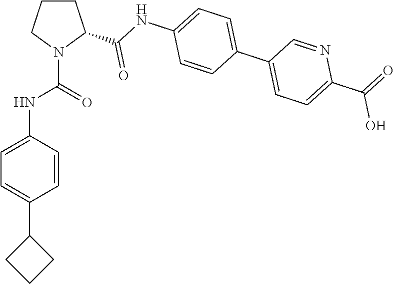 [SMILES: O=C(O)c1ccc(-c2ccc(NC(=O)[C@H]3CCCN3C(=O)Nc3ccc(C4CCC4)cc3)cc2)cn1] | 5-[4-({1-[(4-cyclobutylphenyl)carbamoyl]-D- prolyl}amino)phenyl]pyridine-2-carboxylic acid |
| 212A | 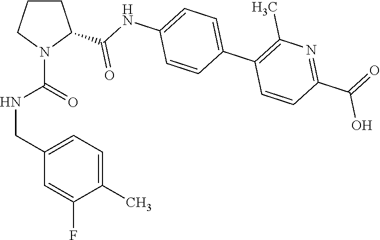 [SMILES: Cc1ccc(CNC(=O)N2CCC[C@@H]2C(=O)Nc2ccc(-c3ccc(C(=O)O)nc3C)cc2)cc1F] | 5-{4-[(1-{[(3-fluoro-4- methylphenyl)methyl]carbamoyl}-D- prolyl)amino]phenyl}-6-methylpyridine-2- carboxylic acid |
| 213A | 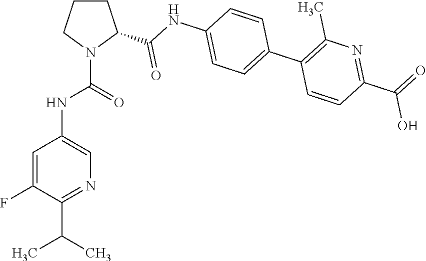 [SMILES: Cc1nc(C(=O)O)ccc1-c1ccc(NC(=O)[C@H]2CCCN2C(=O)Nc2cnc(C(C)C)c(F)c2)cc1] | 5-{4-[(1-{[5-fluoro-6-(propan-2-yl)pyridin-3- yl]carbamoyl}-D-prolyl)amino]phenyl}-6- methylpyridine-2-carboxylic acid |
| 214A | 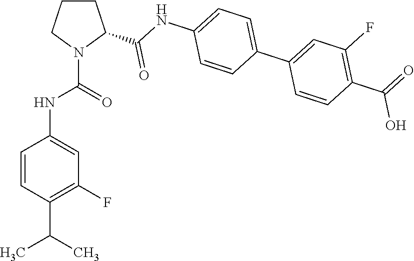 [SMILES: CC(C)c1ccc(NC(=O)N2CCC[C@@H]2C(=O)Nc2ccc(-c3ccc(C(=O)O)c(F)c3)cc2)cc1F] | 3-fluoro-4′-[(1-{[3-fluoro-4-(propan-2- yl)phenyl]carbamoyl}-D-prolyl)amino][1,1′- biphenyl]-4-carboxylic acid |
| 215A | 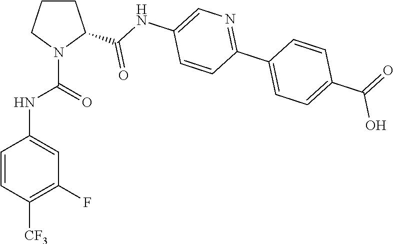 [SMILES: O=C(O)c1ccc(-c2ccc(NC(=O)[C@H]3CCCN3C(=O)Nc3ccc(C(F)(F)F)c(F)c3)cn2)cc1] | 4-{5-[(1-{[3-fluoro-4- (trifluoromethyl)phenyl]carbamoyl}-D- prolyl)amino]pyridin-2-yl}benzoic acid |
| 216A | 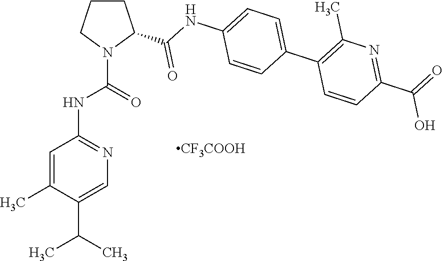 [SMILES: Cc1cc(NC(=O)N2CCC[C@@H]2C(=O)Nc2ccc(-c3ccc(C(=O)O)nc3C)cc2)ncc1C(C)C ; "·CF3COOH"] | 6-methyl-5-{4-[(1-{[4-methyl-5-(propan-2- yl)pyridin-2-yl]carbamoyl}-D- prolyl)amino]phenyl}pyridine-2-carboxylic acid, trifluoroacetate salt |
| 217A | 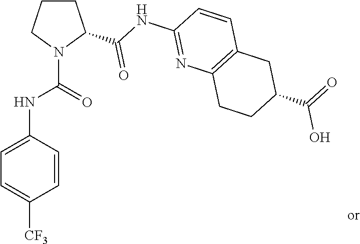 [SMILES: O=C(O)[C@@H]1CCc2nc(NC(=O)[C@H]3CCCN3C(=O)Nc3ccc(C(F)(F)F)cc3)ccc2C1] | 2-[(1-{[4- (trifluoromethyl)phenyl]carbamoyl}-D- prolyl)amino]-5,6,7,8-tetrahydroquinoline- 6-carboxylic acid, DIAST-1 |
| | 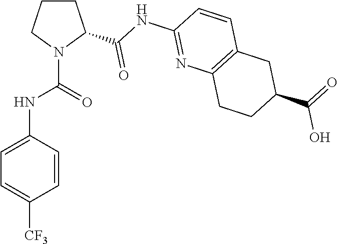 [SMILES: O=C(O)[C@H]1CCc2nc(NC(=O)[C@H]3CCCN3C(=O)Nc3ccc(C(F)(F)F)cc3)ccc2C1] | |
| 218A | 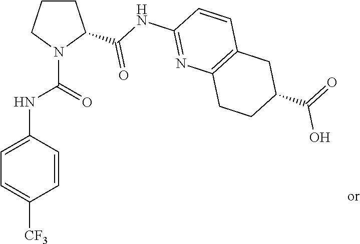 [SMILES: O=C(O)[C@@H]1CCc2nc(NC(=O)[C@H]3CCCN3C(=O)Nc3ccc(C(F)(F)F)cc3)ccc2C1] | 2-[(1-{[4- (trifluoromethyl)phenyl]carbamoyl}-D- prolyl)amino]-5,6,7,8-tetrahydroquinoline- 6-carboxylic acid, DIAST-2 |
| | 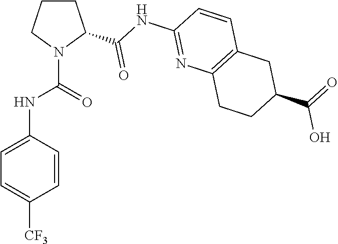 [SMILES: O=C(O)[C@H]1CCc2nc(NC(=O)[C@H]3CCCN3C(=O)Nc3ccc(C(F)(F)F)cc3)ccc2C1] | |
| 219A | 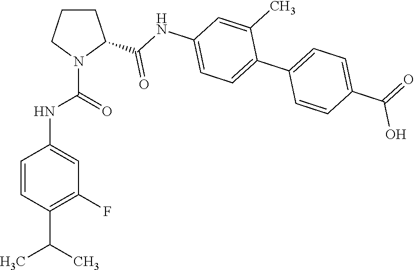 [SMILES: Cc1cc(NC(=O)[C@H]2CCCN2C(=O)Nc2ccc(C(C)C)c(F)c2)ccc1-c1ccc(C(=O)O)cc1] | 4′-[(1-{[3-fluoro-4-(propan-2- yl)phenyl]carbamoyl}-D-prolyl)amino]-2′- methyl[1,1′-biphenyl]-4-carboxylic acid |
| 220A | 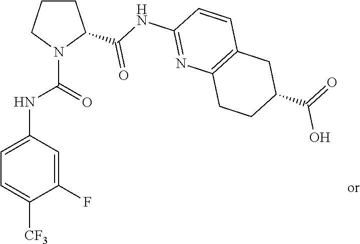 [SMILES: O=C(O)[C@@H]1CCc2nc(NC(=O)[C@H]3CCCN3C(=O)Nc3ccc(C(F)(F)F)c(F)c3)ccc2C1] | 2-[(1-{[3-fluoro-4- (trifluoromethyl)phenyl]carbamoyl}-D- prolyl)amino]-5,6,7,8-tetrahydroquinoline- 6-carboxylic acid, DIAST-1 |
| | 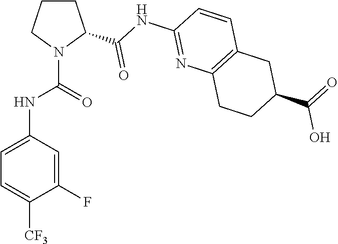 [SMILES: O=C(O)[C@H]1CCc2nc(NC(=O)[C@H]3CCCN3C(=O)Nc3ccc(C(F)(F)F)c(F)c3)ccc2C1] | |
| 221A | 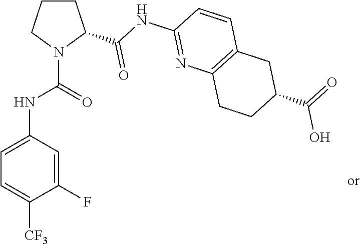 [SMILES: O=C(O)[C@@H]1CCc2nc(NC(=O)[C@H]3CCCN3C(=O)Nc3ccc(C(F)(F)F)c(F)c3)ccc2C1] | 2-[(1-{[3-fluoro-4- (trifluoromethyl)phenyl]carbamoyl}-D- prolyl)amino]-5,6,7,8-tetrahydroquinoline- 6-carboxylic acid, DIAST-2 |
| | 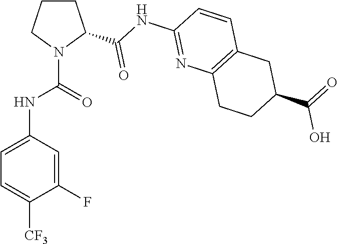 [SMILES: O=C(O)[C@H]1CCc2nc(NC(=O)[C@H]3CCCN3C(=O)Nc3ccc(C(F)(F)F)c(F)c3)ccc2C1] | |
| 222A | 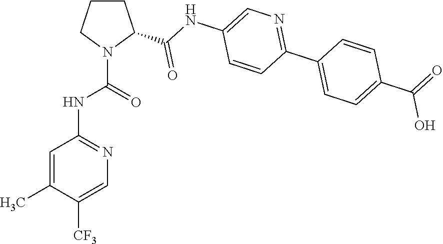 [SMILES: Cc1cc(NC(=O)N2CCC[C@@H]2C(=O)Nc2ccc(-c3ccc(C(=O)O)cc3)nc2)ncc1C(F)(F)F] | 4-{5-[(1-{[4-methyl-5- (trifluoromethyl)pyridin-2-yl]carbamoyl}-D- prolyl)amino]pyridin-2-yl}benzoic acid |
| 223A | 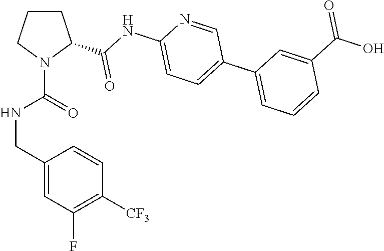 [SMILES: O=C(O)c1cccc(-c2ccc(NC(=O)[C@H]3CCCN3C(=O)NCc3ccc(C(F)(F)F)c(F)c3)nc2)c1] | 3-(6-{[1-({[3-fluoro-4- (trifluoromethyl)phenyl]methyl}carbamoyl)- D-prolyl]amino}pyridin-3-yl)benzoic acid |
| 224A | 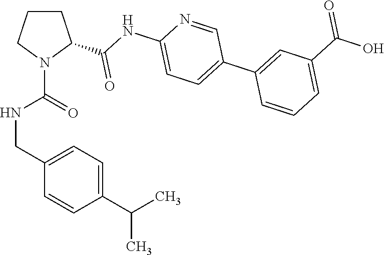 [SMILES: CC(C)c1ccc(CNC(=O)N2CCC[C@@H]2C(=O)Nc2ccc(-c3cccc(C(=O)O)c3)cn2)cc1] | 3-(6-{[1-({[4-(propan-2- yl)phenyl]methyl}carbamoyl)-D- prolyl]amino}pyridin-3-yl)benzoic acid |
| 225A | 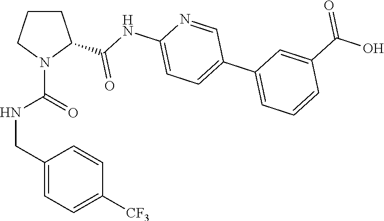 [SMILES: O=C(O)c1cccc(-c2ccc(NC(=O)[C@H]3CCCN3C(=O)NCc3ccc(C(F)(F)F)cc3)nc2)c1] | 3-(6-{[1-({[4- (trifluoromethyl)phenyl]methyl}carbamoyl)- D-prolyl]amino}pyridin-3-yl)benzoic acid |
| 226A | 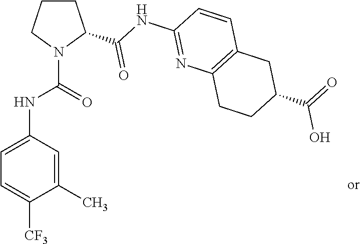 [SMILES: Cc1cc(NC(=O)N2CCC[C@@H]2C(=O)Nc2ccc3c(n2)CC[C@@H](C(=O)O)C3)ccc1C(F)(F)F] | 2-[(1-{[3-methyl-4- (trifluoromethyl)phenyl]carbamoyl}-D- prolyl)amino]-5,6,7,8-tetrahydroquinoline- 6-carboxylic acid, DIAST-1 |
| | 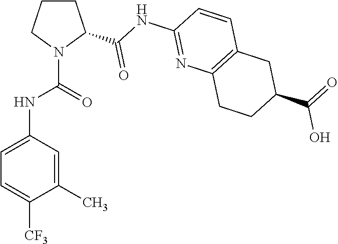 [SMILES: Cc1cc(NC(=O)N2CCC[C@@H]2C(=O)Nc2ccc3c(n2)CC[C@H](C(=O)O)C3)ccc1C(F)(F)F] | |
| 227A | 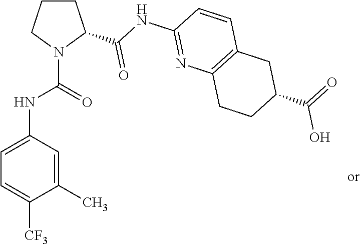 [SMILES: Cc1cc(NC(=O)N2CCC[C@@H]2C(=O)Nc2ccc3c(n2)CC[C@@H](C(=O)O)C3)ccc1C(F)(F)F] | 2-[(1-{[3-methyl-4- (trifluoromethyl)phenyl]carbamoyl}-D- prolyl)amino]-5,6,7,8-tetrahydroquinoline- 6-carboxylic acid, DIAST-2 |
| | 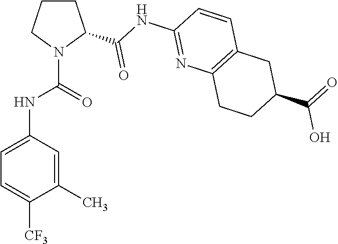 [SMILES: Cc1cc(NC(=O)N2CCC[C@@H]2C(=O)Nc2ccc3c(n2)CC[C@H](C(=O)O)C3)ccc1C(F)(F)F] | |
| 228A | 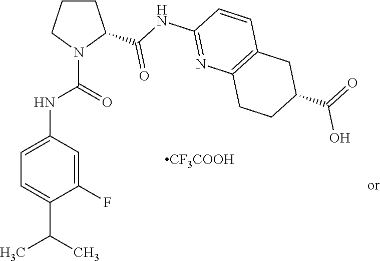 [SMILES: CC(C)c1ccc(NC(=O)N2CCC[C@@H]2C(=O)Nc2ccc3c(n2)CC[C@@H](C(=O)O)C3)cc1F ; "·CF3COOH"] | 2-[(1-{[3-fluoro-4-(propan-2- yl)phenyl]carbamoyl}-D-prolyl)amino]- 5,6,7,8-tetrahydroquinoline-6-carboxylic acid, trifluoroacetate salt, DIAST-1 |
| | 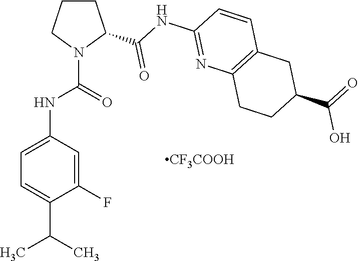 [SMILES: CC(C)c1ccc(NC(=O)N2CCC[C@@H]2C(=O)Nc2ccc3c(n2)CC[C@H](C(=O)O)C3)cc1F ; "·CF3COOH"] | |
| 229A | 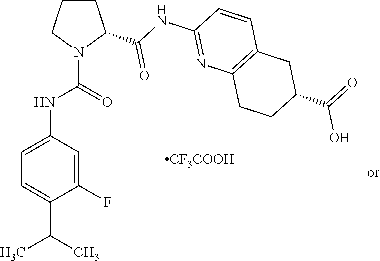 [SMILES: CC(C)c1ccc(NC(=O)N2CCC[C@@H]2C(=O)Nc2ccc3c(n2)CC[C@@H](C(=O)O)C3)cc1F ; "·CF3COOH"] | 2-[(1-{[3-fluoro-4-(propan-2- yl)phenyl]carbamoyl}-D-prolyl)amino]- 5,6,7,8-tetrahydroquinoline-6-carboxylic acid, trifluoroacetate salt, DIAST-2 |
| | 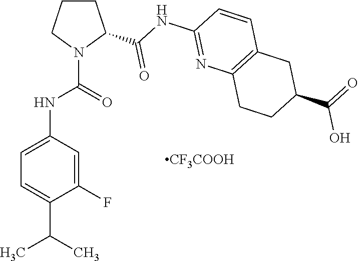 [SMILES: CC(C)c1ccc(NC(=O)N2CCC[C@@H]2C(=O)Nc2ccc3c(n2)CC[C@H](C(=O)O)C3)cc1F ; "·CF3COOH"] | |

### GLP-1R Agonist Compounds

In one embodiment, the GLP-1R agonist compound is a compound of Formula B-I:

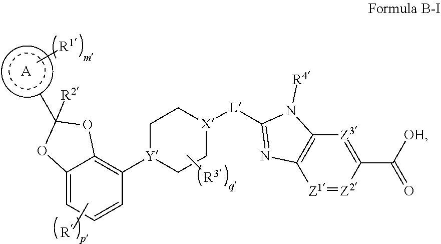

or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O}, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}, and —N(R^{N}′)₂
R^{5′} is a 4- to 6-membered heterocycloalkyl, wherein said heterocycloalkyl may be substituted with 0 to 2 substituents as valency allows independently selected from:
   0 to 1 oxo (═O),
   0 to 1 —CN,
   0 to 2 F atoms, and
   0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O}′;
R^{6′} is a 5- to 6-membered heteroaryl, wherein said heteroaryl may be substituted with 0 to 2 substituents as valency allows independently selected from:
   0 to 2 halogens,
   0 to 1 substituent selected from —OR^{O}′ and —N(R^{N}′)₂, and
   0 to 2 —C₁₋₃alkyl, wherein the alkyl may be substituted with 0 to 3 substituents as valency allows independently selected from:
   0 to 3 F atoms, and
   0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z}′, or one of Z¹′, Z²′, and Z³′ is N and the other two are —CR^{Z}′;
and
each R^{Z}′ is independently H, F, Cl, or —CH₃.

In one embodiment, the GLP-1R compound is a compound of Formula B-II:

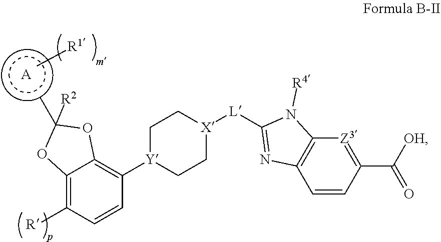

or a pharmaceutically acceptable salt thereof, wherein
R′ is F;
p′ is 0 or 1;
ring A is phenyl or pyridinyl;
m′ is 0, 1, or 2;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or CH₃;
X′-L′ is N—CH₂, or cyclopropyl;
Y′ is CH or N;
Z^{3′} is —CR^{Z′} or N; and
R^{Z′} is H, F, Cl, or —CH₃.

In one embodiment, the GLP-1R compound is a compound of Formula B-III:

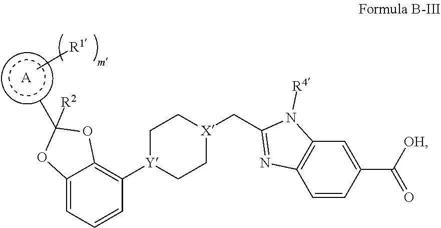

or a pharmaceutically acceptable salt thereof, wherein
ring A is phenyl or pyridinyl;
m′ is 0, 1, or 2;
each R^{1′} is independently selected from F, Cl, and —CN;
R^{2′} is H or CH₃; and
Y′ is CH or N.

In one embodiment, the GLP-1R compound is a compound of Formula B-IV:

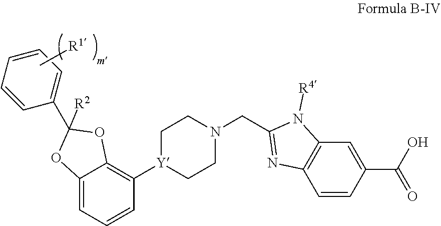

or a pharmaceutically acceptable salt thereof, wherein
m′ is 0, 1, or 2;
each R^{1′} is independently selected from F, Cl, and —CN;
R^{2′} is H or CH₃; and
Y′ is CH or N.

In one embodiment, the GLP-1R compound is a compound of Formula B-V

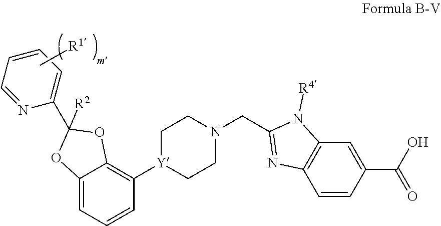

or a pharmaceutically acceptable salt thereof, wherein
m′ is 0 or 1;
R^{1′} is F, Cl, or —CN;
R^{2′} is H or CH₃; and
Y′ is CH or N.

In another embodiment, the GLP-1R compound is a compound of Formula B-IV or B-V, or a pharmaceutically acceptable salt thereof, wherein the phenyl or pyridinyl of Ring A has one R^{1′} para substituted relative to carbon of said phenyl or pyridinyl attached to the dioxolane to provide:

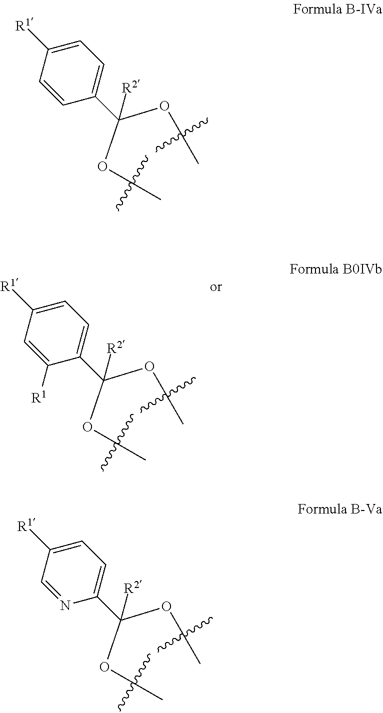

wherein
each R^{1′} is independently selected from F, Cl, and —CN;
R^{2′} is H or CH₃; and
Y′ is CH or N.

Another embodiment concerns compounds of other embodiments herein, e.g., compounds of Formulas B-I or B-II, or a pharmaceutically acceptable salt thereof, wherein X′-L′ is N—CH₂; and Y′ is CH or N. From the embodiments described herein, in such a case, X is N and L′ is CH₂.

Another embodiment concerns compounds of other embodiments herein, e.g., compounds of Formulas B-I, or B-II, or a pharmaceutically acceptable salt thereof, wherein X′-L′ is CHCH₂; and Y′ is N. From the embodiments described herein, in such a case, X is CH and L′ is CH₂.

Another embodiment concerns compounds of other embodiments herein, e.g., compounds of Formulas B-I, or B-II, or a pharmaceutically acceptable salt thereof, wherein X′-L′ is CHCH₂; and Y′ is CH. From the embodiments described herein, in such a case, X is CH and L′ is CH₂.

Another embodiment concerns compounds of other embodiments herein, e.g., compounds of Formulas B-I, or B-II, or a pharmaceutically acceptable salt thereof, wherein X′-L′ is cyclopropyl; and Y′ is N.

In the embodiments where X′-L′ is cyclopropyl, the compounds of Formulas B-I, or B-II would provide:

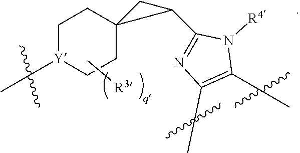

Another embodiment concerns compounds of Formulas B-I, B-II, B-III, B-IV, or B-V, wherein R^{4′} is —CH₂CH₂OCH₃, C₁₋₃alkylene-R⁵, or C₁₋₃alkylene-R^{6′}, or a pharmaceutically acceptable salt thereof.

Another embodiment concerns compounds of Formulas B-II, B-III, B-IV, or B-V, wherein R^{4′} is as defined for compounds of Formula B-I, or a pharmaceutically acceptable salt thereof.

Another embodiment concerns compounds of Formulas B-I, B-II, B-III, B-IV, or B-V, wherein R^{4′} is —C₁₋₃alkyl, wherein said alkyl may be substituted as valency allows with 0 to 1 substituent selected from —C₀₋₁alkylene-OR^{O}, and —N(R^{N}′)₂, or a pharmaceutically acceptable salt thereof.

Another embodiment concerns compounds of Formulas B-I, B-II, B-III, B-IV, or B-V, wherein R^{4′} is —(CH₂)₂OCH₃, or —(CH₂)₂N(CH₃)₂, or a pharmaceutically acceptable salt thereof.

Another embodiment concerns compounds of Formulas B-I, B-II, B-III, B-IV, or B-V, wherein R^{4′} is —CH₂—R^{5′}, wherein R^{5′} is the 4- to 5-membered heterocycloalkyl, wherein said heterocycloalkyl may be substituted with 0 to 2 substituents as valency allows independently selected from: 0 to 2 F atoms, and 0 to 1 substituent selected from —OCH₃ and —CH₂OCH₃; or a pharmaceutically acceptable salt thereof.

Another embodiment concerns compounds of Formulas B-I, B-II, B-III, B-IV, or B-V, wherein the heterocycloalkyl is

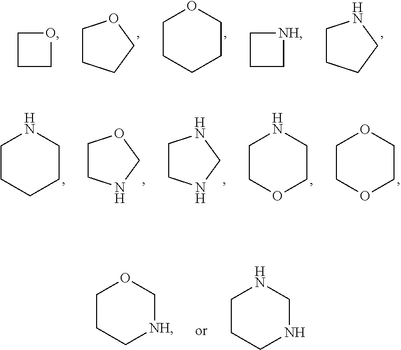

wherein the heterocycloalkyl may be substituted with 0 to 2 substituents as valency allows, e.g., replacing hydrogen, independently selected from:
0 to 1 oxo (O═),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be independently substituted with 0 to 3 substituents as valency allows independently selected from:
   0 to 3 F atoms,
   0 to 1 —CN, and
   0 to 1 —OR^{O},
      or a pharmaceutically acceptable salt thereof.

Another embodiment concerns compounds of Formulas B-I, B-II, B-III, B-IV, or B-V, wherein the heterocycloalkyl is

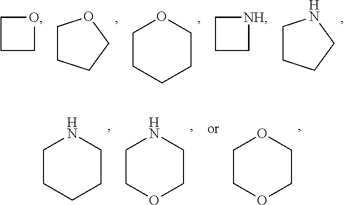

wherein the heterocycloalkyl may be substituted with 0 to 2 substituents as valency allows, e.g., replacing hydrogen, independently selected from:
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be independently substituted with 0 to 3 substituents as valency allows independently selected from:
   0 to 3 F atoms,
   0 to 1 —CN, and
   0 to 1 —OR^{O}′, or a pharmaceutically acceptable salt thereof.

Another embodiment concerns compounds of Formulas B-I, B-II, B-III, B-IV, or B-V, wherein the heterocycloalkyl is

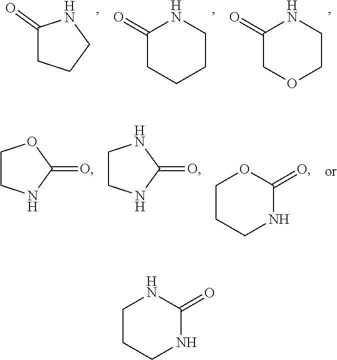

wherein the heterocycloalkyl may be substituted with 0 to 1 substituent as valency allows, e.g., replacing hydrogen, selected from:
—CN,
F atom, and
0 to 1 substituent independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O}′,
      or a pharmaceutically acceptable salt thereof.

Another embodiment concerns compounds of Formulas B-I, B-II, B-III, B-IV, or B-V, wherein the heterocycloalkyl is

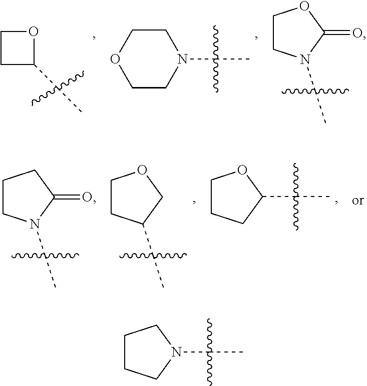

or a pharmaceutically acceptable salt thereof. Another embodiment concerns compounds of Formulas B-I, B-II, B-III, B-IV, or B-V, wherein the heterocycloalkyl is

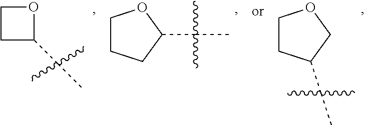 [SMILES: *[C@@H]1CCO1 ; *[C@@H]1CCCO1 ; *[C@@H]1CCOC1]

wherein the heterocycloalkyl may be substituted as valency allows with 0 to 1 methyl, wherein said methyl may be substituted with 0 to 3 F atoms, or a pharmaceutically acceptable salt thereof. Another embodiment concerns compounds of Formulas B-I, B-II, B-III, B-IV, or B-V, wherein the heterocycloalkyl is

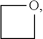

wherein the heterocycloalkyl is unsubstituted.

Another embodiment concerns compounds of Formulas B-I, B-II, B-III, B-IV, or B-V, wherein —CH₂—R^{5′} and the nitrogen to which R^{4′} is attached provides:

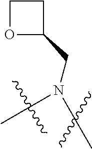 [SMILES: *N(*)C[C@@H]1CCO1]

or a pharmaceutically acceptable salt thereof.

Another embodiment concerns compounds of Formulas B-I, B-II, B-III, B-IV, or B-V, wherein
R⁴ is —CH₂—R^{6′}, wherein R^{6′} is the 5-membered heteroaryl, wherein said heteroaryl may be substituted with 0 to 2 substituents as valency allows independently selected from:
0 to 2 halogens, wherein the halogen is independently selected from F and Cl,
0 to 1 —OCH₃, and
0 to 1 —CH₃, —CH₂CH₃, —CF₃, or —CH₂CH₂OCH₃;
or a pharmaceutically acceptable salt thereof.

Another embodiment concerns compounds of Formulas B-I, B-II, B-III, B-IV, or B-V, wherein the heteroaryl is

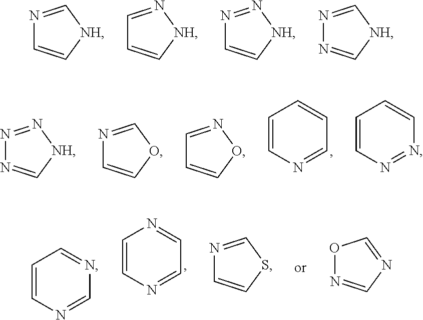

wherein said heteroaryl may be substituted with 0 to 2 substituents as valency allows, e.g., replacing hydrogen, independently selected from:
0 to 2 halogens, wherein the halogen is independently selected from F and C,
0 to 1 substituent selected from —OR^{O}′ and —N(R^{N}′)₂, or
0 to 2 —C₁₋₃alkyl, wherein the alkyl may be substituted with 0 to 3 substituents as valency allows independently selected from:
   0 to 3 F atoms, and
   0 to 1 —OR^{O}′;
      or a pharmaceutically acceptable salt thereof.

Another embodiment concerns compounds of Formulas B-I, B-II, B-III, B-IV, or B-V, wherein the heteroaryl is

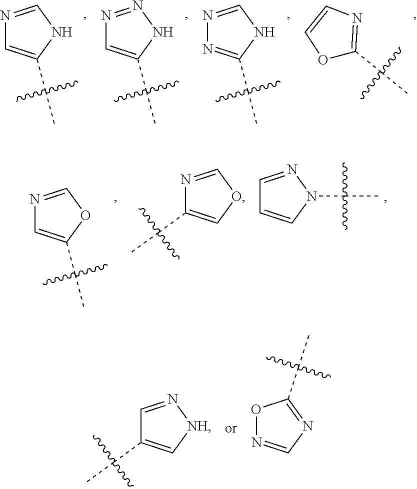

wherein said heteroaryl may be substituted with 0 to 2 substituents as valency allows, e.g., replacing hydrogen, independently selected from:
0 to 2 halogens, wherein the halogen is independently selected from F and C,
0 to 1 substituent selected from —OR^{O}′ and —N(R^{N}′)₂, or
0 to 2 —C₁₋₃alkyl, wherein the alkyl may be substituted with 0 to 3 substituents as valency allows independently selected from:
   0 to 3 F atoms, and
   0 to 1 —OR^{O′};
      or a pharmaceutically acceptable salt thereof.

Another embodiment concerns compounds of Formulas B-I, B-II, B-III, B-IV, or B-V, wherein the heteroaryl is

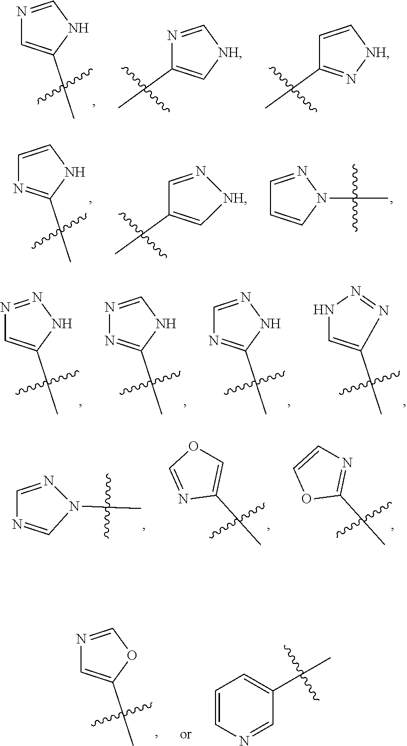

wherein said heteroaryl may be substituted with 0 to 1 substituent as valency allows with —C₁₋₂alkyl, wherein the alkyl may be substituted with 0 to 3 substituents as valency allows independently selected from:
   0 to 3 F atoms, and
   0 to 1 —OR^{O′}; and
each R^{O}′ is independently H, or —C₁₋₃alkyl;
   or a pharmaceutically acceptable salt thereof. One will recognize that any substituent would replace H on the carbon or nitrogen being substituted. A non-limiting example of substituted heteroaryls are:

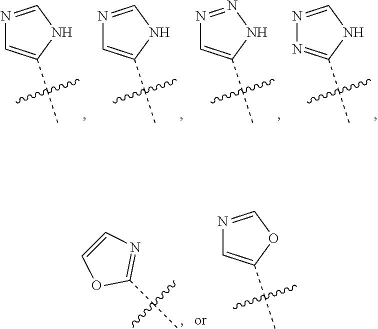

One will recognize that H is replaced with a substituent, e.g., R^{6s} (substituent allowed on any heteroaryl of R⁶), to provide:

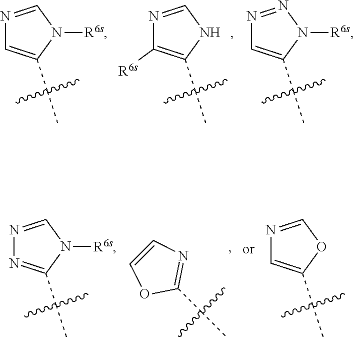

wherein R^{6s} is —C₁₋₂alkyl, wherein the alkyl may be substituted with 0 to 3 substituents as valency allows independently selected from:
0 to 3 F atoms, and
0 to 1 —OR^{O′}; and
each R^{O} is independently H, or —C₁₋₃alkyl;
or a pharmaceutically acceptable salt thereof.

Another embodiment concerns compounds of Formulas B-I, B-II, B-III, B-IV, or B-V, wherein the heteroaryl is

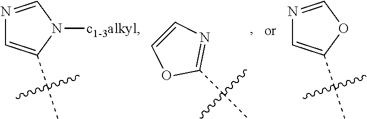

or a pharmaceutically acceptable salt thereof.

Another embodiment concerns compounds of other embodiments herein, e.g., compounds of Formulas B-I, B-II, B-III, B-IV, or B-V, wherein Z^{1′}, Z^{2′}, and Z^{3′} are each CR^{Z}′, or a pharmaceutically acceptable salt thereof. Another embodiment concerns compounds of other embodiments herein, e.g., compounds of Formulas B-I, B-II, B-III, B-IV, or B-V, wherein R^{Z}′ is H, or a pharmaceutically acceptable salt thereof. Another embodiment concerns compounds of other embodiments herein, e.g., compounds of Formulas B-I, B-II, B-III, B-IV, or B-V, wherein Z^{1′}, Z^{2′}, and Z^{3′} are each CH, or a pharmaceutically acceptable salt thereof.

Another embodiment concerns compounds of other embodiments herein, e.g., compounds of Formulas B-I, B-II, B-III, B-IV, or B-V, wherein R^{3′} is —CH₃, or —CF₃; and q is 1, or a pharmaceutically acceptable salt thereof.

Another embodiment concerns compounds of other embodiments herein, e.g., compounds of Formulas B-I, B-II, B-III, B-IV, or B-V, wherein each R^{1′} is independently F, Cl, or —CN, or a pharmaceutically acceptable salt thereof.

Another embodiment concerns compounds of other embodiments herein, e.g., compounds of Formulas B-I, B-II, B-III, B-IV, or B-V, wherein R^{4′} is —CH₂—R^{5′}, or a pharmaceutically acceptable salt thereof.

Another embodiment concerns compounds of other embodiments herein, e.g., compounds of Formulas B-I, B-II, B-III, B-IV, or B-V, wherein R^{4′} is —CH₂—R^{6′}, or a pharmaceutically acceptable salt thereof.

Another embodiment concerns compounds of other embodiments herein, e.g., compounds of Formulas B-I, B-II, B-III, B-IV, or B-V, wherein the compound is the free acid.

Another embodiment concerns any embodiment of compounds of Formulas B-I, B-II, B-III, B-IV, or B-V, wherein Ring A and R^{2′} provide:

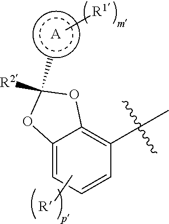

or a pharmaceutically acceptable salt thereof, wherein
R′ is F, Cl, or —CN;
p′ is or 1;
m′ is 0, 1, or 2; and
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms.

Another embodiment concerns compounds of Formulas B-I, B-II, B-III, B-IV, or B-V, wherein R^{2′} is H, or a pharmaceutically acceptable salt thereof.

Another embodiment concerns compounds on the invention, wherein the compound is
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid; or
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
   or a pharmaceutically acceptable salt thereof.

Another embodiment concerns compounds on the invention, wherein the compound is
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid; or
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
   or a pharmaceutically acceptable salt thereof.

Another embodiment concerns compounds on the invention, wherein R² is CH₃, or a pharmaceutically acceptable salt thereof.

Another embodiment concerns compounds on the invention, wherein the compound is
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid; or
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
   or a pharmaceutically acceptable salt thereof.

Another embodiment concerns compounds on the invention, wherein the compound is
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid; or
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
   or a pharmaceutically acceptable salt thereof.

Another embodiment concerns compounds on the invention, wherein the compound is
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; or
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
   or a pharmaceutically acceptable salt thereof.

Another embodiment concerns compounds on the invention, wherein the compound is
2-({4-[(2R)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; or
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
   or a pharmaceutically acceptable salt thereof.

Another embodiment includes a compound that is 2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid, or a pharmaceutically acceptable salt thereof, wherein the salt is a tris salt.

Another embodiment includes a compound that is 2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid, as a free acid.

Another embodiment includes a compound that is

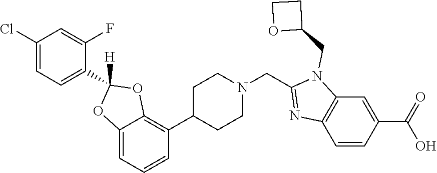 [SMILES: O=C(O)c1ccc2nc(CN3CCC(c4cccc5c4O[C@@H](c4ccc(Cl)cc4F)O5)CC3)n(C[C@@H]3CCO3)c2c1]

or a pharmaceutically acceptable salt thereof.

Another embodiment includes a compound that is 2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid, or a pharmaceutically acceptable salt, wherein the salt is a tris salt {the tris salt of this compound is also known as: 1,3-dihydroxy-2-(hydroxymethyl)propan-2-aminium 2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylate}.

In some embodiments, the present invention provides a crystal form of anhydrous tris salt of 2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid. In some further embodiments, the crystal form of anhydrous (anhydrate) tris salt of 2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid is designated as “Form I” that is characterized according to its unique solid state signatures with respect to, for example, powder X-ray diffraction (PXRD). In some embodiments, Form I exhibits a powder X-ray diffraction pattern comprising at least two characteristic peaks, in terms of 2θ, selected from at 3.7±0.2°; 7.3±0.2°; 8.5±0.2°; 10.1±0.2°; 14.7±0.2°; and 16.9±0.2°. In some embodiments, Form I exhibits a powder X-ray diffraction pattern comprising at least three characteristic peaks, in terms of 2θ, selected from at 3.7±0.2; 7.3±0.2°; 8.5±0.2°; 10.1±0.2°; 14.7±0.2°; and 16.9±0.2°. In some embodiments, Form I exhibits a powder X-ray diffraction pattern comprising at least four characteristic peaks, in terms of 2θ, selected from at 3.7±0.2°; 7.3±0.2°; 8.5±0.2°; 10.1±0.2°; 14.7±0.2°; and 16.9±0.2°. In some embodiments, Form I exhibits a powder X-ray diffraction pattern comprising at least five characteristic peaks, in terms of 2θ, selected from at 3.7±0.2°; 7.3±0.2°; 8.5±0.2°; 10.1±0.2°; 14.7±0.2°; and 16.9±0.2°.

In some embodiments, Form I exhibits a powder X-ray diffraction pattern comprising characteristic peaks, in terms of 2θ, at 3.7±0.2° and 7.3±0.2°. In some embodiments, Form I exhibits a powder X-ray diffraction pattern comprising peaks, in terms of 2θ, at 3.7±0.2°; 7.3±0.2°; and 14.7±0.2°. In some further embodiments, Form I exhibits the X-ray powder diffraction pattern further comprises at least one peak, in terms of 2θ, selected from at 8.5±0.2°; 10.1±0.2°; and 16.9±0.2°. In some embodiments, Form I exhibits a powder X-ray diffraction pattern comprising peaks, in terms of 2θ, at 3.7±0.2°; 7.3±0.2°; 14.7±0.2°; and 16.9±0.2°. In some embodiments, Form I exhibits a powder X-ray diffraction pattern comprising peaks, in terms of 2θ, at 3.7±0.2; 7.3±0.2°; 8.5±0.2°; 10.1±0.2°; 14.7±0.2°; and 16.9±0.2°.

As is well known in the art of powder diffraction, the relative intensities of the peaks (reflections) can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

Another embodiment includes a compound that is 2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid, as a free acid.

Another embodiment includes a compound that is

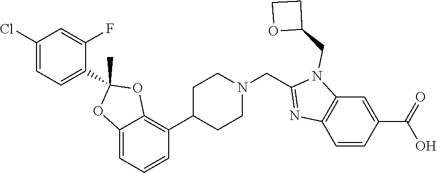 [SMILES: C[C@]1(c2ccc(Cl)cc2F)Oc2cccc(C3CCN(Cc4nc5ccc(C(=O)O)cc5n4C[C@@H]4CCO4)CC3)c2O1]

or a pharmaceutically acceptable salt thereof.

Another embodiment includes a compound that is
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; or
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; as the free acid.

Another embodiment includes a compound that is
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; or
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid; or a pharmaceutically acceptable salt thereof, wherein the salt is a tris salt.

Another embodiment includes a compound that is 2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2:

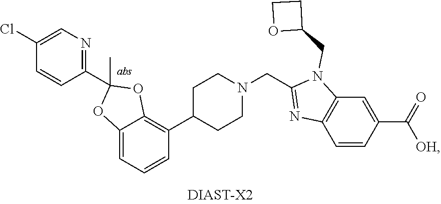 [SMILES: CC1(c2ccc(Cl)cn2)Oc2cccc(C3CCN(Cc4nc5ccc(C(=O)O)cc5n4C[C@@H]4CCO4)CC3)c2O1]

or pharmaceutically acceptable salt thereof. In some further embodiments, the present invention provides a compound that is 2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2, or tris salt [i.e. 1,3-dihydroxy-2-(hydroxymethyl)propan-2-amine salt] thereof. The chiral center on the left part of the compound structure is marked as “abs” to indicate that chiral center has only one stereo-configuration (i.e., not a racemate with respect to that chiral center).

In some embodiments, the present invention provides a crystal form of anhydrous tris salt of 2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2. In some further embodiments, the crystal form of anhydrous (anhydrate) tris salt of 2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2, is designated as “Form A” that is characterized according to its unique solid state signatures with respect to, for example, powder X-ray diffraction (PXRD). In some embodiments, Form A exhibits a powder X-ray diffraction pattern comprising at least two characteristic peaks, in terms of 2θ, selected from at 7.7±0.2°; 15.2±0.2°; 15.7±0.2°; and 17.6±0.2°. In some embodiments, Form A exhibits a powder X-ray diffraction pattern comprising at least three characteristic peaks, in terms of 2θ, selected from at 7.7±0.2°; 15.2±0.2°; 15.7±0.2°; and 17.6±0.2°. In some embodiments, Form A exhibits a powder X-ray diffraction pattern comprising characteristic peaks, in terms of 2θ, selected from at 7.7±0.2°; 15.2±0.2°; 15.7±0.2°; and 17.6±0.2°.

In some embodiments, Form I exhibits a powder X-ray diffraction pattern comprising characteristic peaks, in terms of 2θ, at 7.7±0.2° and 17.6±0.2°. In some embodiments, Form A exhibits a powder X-ray diffraction pattern comprising peaks, in terms of 2θ, at 7.7±0.2°; 15.2±0.2°; and 17.6±0.2°. In some embodiments, Form I exhibits a powder X-ray diffraction pattern comprising peaks, in terms of 2θ, at 7.7±0.2°; 15.2±0.2°; and 15.7±0.2°. In some embodiments, Form I exhibits a powder X-ray diffraction pattern comprising peaks, in terms of 2θ, at 7.7±0.2°; 15.2±0.2; 15.7±0.2°; and 17.6±0.2°.

As is well known in the art of powder diffraction, the relative intensities of the peaks (reflections) can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

The GLP-1R antagonist compounds of the disclosure are compounds comprising a molecular weight of from about 400 Da to about 700 Da, from about 400 Da to about 500 Da, from about 500 Da to about 600 Da, from about 600 Da to about 700 Da, from about 450 Da to about 550 Da, from about 400 Da to about 450 Da, from about 450 Da to about 500 Da, from about 500 Da to about 550 Da, from about 550 Da to about 600 Da, from about 600 Da to about 650 Da, and from about 650 Da to about 700 Da. In some embodiments, the GLP-1R antagonist compounds of the disclosure comprise a molecular weight of from about 450 Da to about 500 Da. In some embodiments, the GLP-1R antagonist compounds of the disclosure comprise a molecular weight of from about 500 Da to about 550 Da. In some embodiments, the GLP-1R antagonist compounds of the disclosure comprise a molecular weight of from about 550 Da to about 600 Da.

TABLE 2 shows GLP-1R agonists compounds of the disclosure that can be used in combination with the GIPR antagonist compounds disclosed herein.

TABLE 2
GLP-1R Agonist compounds for GIPR antagonist combination therapy

| GLP1R | | |
|---|---|---|
| Ag | Structure | IUPAC Name |
| 1B | 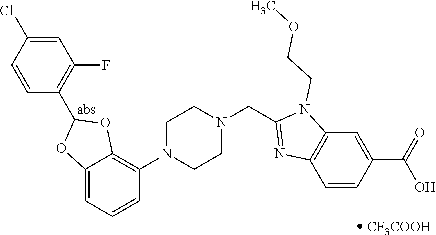 [SMILES: COCCn1c(CN2CCN(c3cccc4c3OC(c3ccc(Cl)cc3F)O4)CC2)nc2ccc(C(=O)O)cc21 ; "· CF3COOH"] | 2-({4-[2-(4-Chloro-2-fluorophenyl)-1,3- benzodioxol-4-yl]piperazin-1-yl}methyl)-1- (2-methoxyethyl)-1H-benzimidazole-6- carboxylic acid, ENT-X1, trifluoroacetate salt |
| | ENT-X1 | |
| 2B | 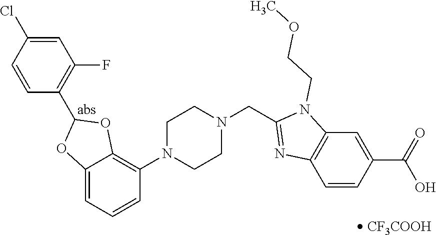 [SMILES: COCCn1c(CN2CCN(c3cccc4c3OC(c3ccc(Cl)cc3F)O4)CC2)nc2ccc(C(=O)O)cc21 ; "· CF3COOH"] | 2-({4-[2-(4-Chloro-2-fluorophenyl)-1,3- benzodioxol-4-yl]piperazin-1-yl}methyl)-1- (2-methoxyethyl)-1H-benzimidazole-6- carboxylic acid, ENT-X2, trifluoroacetate salt |
| | ENT-X2 | |
| 3B | 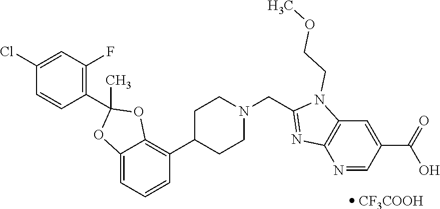 [SMILES: COCCn1c(CN2CCC(c3cccc4c3OC(C)(c3ccc(Cl)cc3F)O4)CC2)nc2ncc(C(=O)O)cc21 ; "· CF3COOH"] | 2-({4-[2-(4-Chloro-2-fluorophenyl)-2- methyl-1,3-benzodioxol-4-yl]piperidin-1- yl}methyl)-1-(2-methoxyethyl)-1H- imidazo[4,5-b]pyridine-6-carboxylic acid, trifluoroacetate salt |
| 4B | 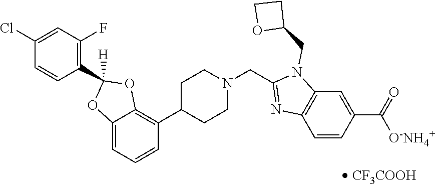 | Ammonium 2-({4-[(2R)-2-(4-chloro-2- fluorophenyl)-1,3-benzodioxol-4- yl]piperidin-1-yl}methyl)-1-[(2S)-oxetan-2- ylmethyl]-1H-benzimidazole-6-carboxylate |
| 5B | 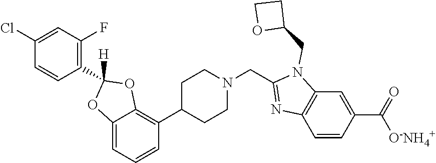 | Ammonium 2-({4-[(2S)-2-(4-chloro-2- fluorophenyl)-1,3-benzodioxol-4- yl]piperidin-1-yl}methyl)-1-[(2S)-oxetan-2- ylmethyl]-1H-benzimidazole-6-carboxylate |
| 6B | 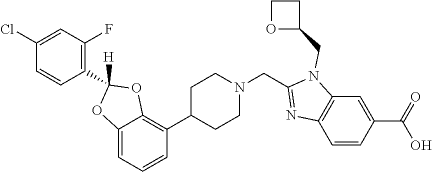 [SMILES: O=C(O)c1ccc2nc(CN3CCC(c4cccc5c4O[C@@H](c4ccc(Cl)cc4F)O5)CC3)n(C[C@@H]3CCO3)c2c1] | 2-({4-[(2S)-2-(4-Chloro-2-fluorophenyl)-1,3- benzodioxol-4-yl]piperidin-1-yl}methyl)-1- [(2S)-oxetan-2-ylmethyl]-1H- benzimidazole-6-carboxylic acid |
| 7B | 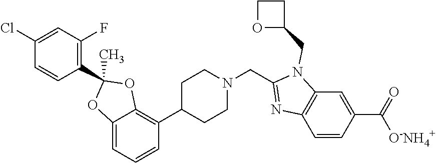 | Ammonium 2-({4-[(2R)-2-(4-chloro-2- fluorophenyl)-2-methyl-1,3-benzodioxol-4- yl]piperidin-1-yl}methyl)-1-[(2S)-oxetan-2- ylmethyl]-1H-benzimidazole-6-carboxylate |
| 8B | 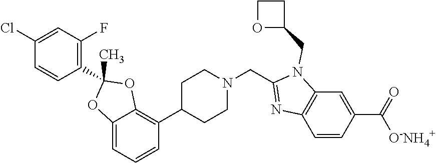 | Ammonium 2-({4-[(2S)-2-(4-chloro-2- fluorophenyl)-2-methyl-1,3-benzodioxol-4- yl]piperidin-1-yl}methyl)-1-[(2S)-oxetan-2- ylmethyl]-1H-benzimidazole-6-carboxylate |
| 9B | 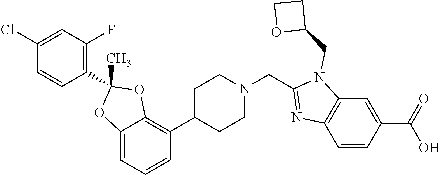 [SMILES: C[C@]1(c2ccc(Cl)cc2F)Oc2cccc(C3CCN(Cc4nc5ccc(C(=O)O)cc5n4C[C@@H]4CCO4)CC3)c2O1] | 2-({4-[(2S)-2-(4-Chloro-2-fluorophenyl)-2- methyl-1,3-benzodioxol-4-yl]piperidin-1- yl}methyl)-1-[(2S)-oxetan-2-ylmethyl]-1H- benzimidazole-6-carboxylic acid |
| 10B | 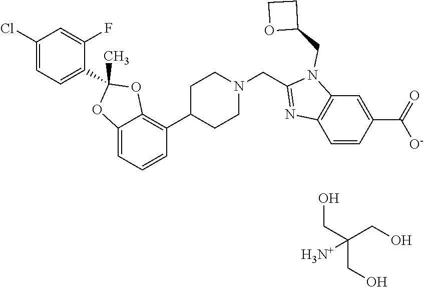 [SMILES: C[C@]1(c2ccc(Cl)cc2F)Oc2cccc(C3CCN(Cc4nc5ccc(C(=O)[O-])cc5n4C[C@@H]4CCO4)CC3)c2O1 ; [NH3+]C(CO)(CO)CO] | 2-({4-[(2S)-2-(4-Chloro-2-fluorophenyl)-2- methyl-1,3-benzodioxol-4-yl]piperidin-1- yl}methyl)-1-[(2S)-oxetan-2-ylmethyl]-1H- benzimidazole-6-carboxylic acid 1,3- dihydroxy-2-(hydroxymethyl)propan-2- aminium salt |
| 11B | 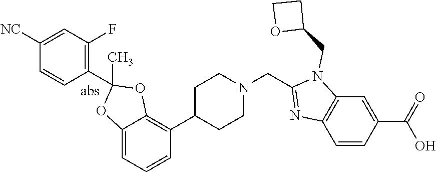 [SMILES: CC1(c2ccc(C#N)cc2F)Oc2cccc(C3CCN(Cc4nc5ccc(C(=O)O)cc5n4C[C@@H]4CCO4)CC3)c2O1] | 2-({4-[2-(4-Cyano-2-fluorophenyl)-2- methyl-1,3-benzodioxol-4-yl]piperidin-1- yl}methyl)-1-[(2S)-oxetan-2-ylmethyl]-1H- benzimidazole-6-carboxylic acid, DIAST- X1 |
| | DIAST-X1 | |
| 12B | 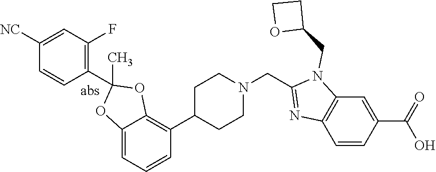 [SMILES: CC1(c2ccc(C#N)cc2F)Oc2cccc(C3CCN(Cc4nc5ccc(C(=O)O)cc5n4C[C@@H]4CCO4)CC3)c2O1] | 2-({4-[2-(4-Cyano-2-fluorophenyl)-2- methyl-1,3-benzodioxol-4-yl]piperidin-1- yl}methyl)-1-[(2S)-oxetan-2-ylmethyl]-1H- benzimidazole-6-carboxylic acid, DIAST- X2 |
| | DIAST-X2 | |
| 13B | 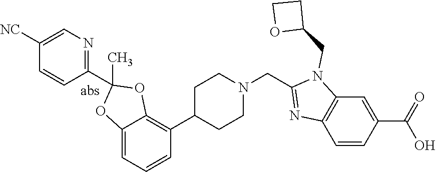 [SMILES: CC1(c2ccc(C#N)cn2)Oc2cccc(C3CCN(Cc4nc5ccc(C(=O)O)cc5n4C[C@@H]4CCO4)CC3)c2O1] | 2-({4-[2-(5-Chloropyridin-2-yl)-2-methyl- 1,3-benzodioxol-4-yl]piperidin-1-yl}methyl)- 1-[(2S)-oxetan-2-ylmethyl]-1H- benzimidazole-6-carboxylic acid, DIAST- X2 |
| | DIAST-X2 | |
| 14B | 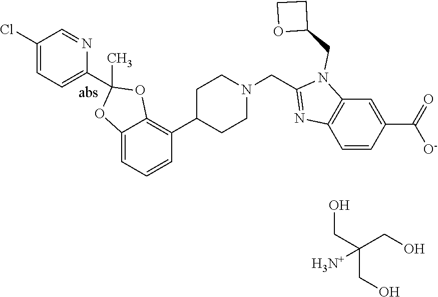 [SMILES: CC1(c2ccc(Cl)cn2)Oc2cccc(C3CCN(Cc4nc5ccc(C(=O)[O-])cc5n4C[C@@H]4CCO4)CC3)c2O1 ; [NH3+]C(CO)(CO)CO] | 2-({4-[2-(5-Chloropyridin-2-yl)-2-methyl- 1,3-benzodioxol-4-yl]piperidin-1-yl}methyl)- 1-[(2S)-oxetan-2-ylmethyl]-1H- benzimidazole-6-carboxylic acid 1,3- dihydroxy-2-(hydroxymethyl)propan-2- aminium salt |
| | DIAST-X2 | |
| 15B | 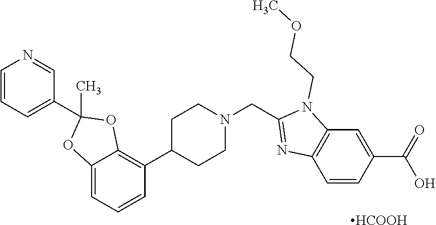 [SMILES: COCCn1c(CN2CCC(c3cccc4c3OC(C)(c3cccnc3)O4)CC2)nc2ccc(C(=O)O)cc21 ; "·HCOOH"] | 1-(2-Methoxyethyl)-2-({4-[2-methyl-2- (pyridin-3-yl)-1,3-benzodioxol-4- yl]piperidin-1-yl}methyl)-1H-benzimidazole- 6-carboxylic acid, formate salt |
| 16B | 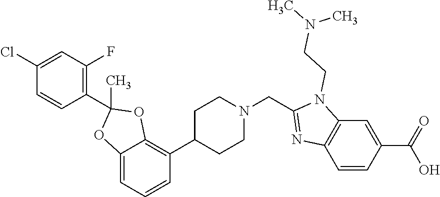 [SMILES: CN(C)CCn1c(CN2CCC(c3cccc4c3OC(C)(c3ccc(Cl)cc3F)O4)CC2)nc2ccc(C(=O)O)cc21] | 2-({4-[2-(4-Chloro-2-fluorophenyl)-2- methyl-1,3-benzodioxol-4-yl]piperidin-1- yl}methyl)-1-[2-(dimethylamino)ethyl]-1H- benzimidazole-6-carboxylic acid |
| 17B | 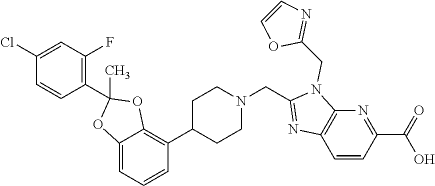 [SMILES: CC1(c2ccc(Cl)cc2F)Oc2cccc(C3CCN(Cc4nc5ccc(C(=O)O)nc5n4Cc4ncco4)CC3)c2O1] | 2-({4-[2-(4-Chloro-2-fluorophenyl)-2- methyl-1,3-benzodioxol-4-yl]piperidin-1- yl}methyl)-3-(1,3-oxazol-2-ylmethyl)-3H- imidazo[4,5-b]pyridine-5-carboxylic acid |
| 18B | 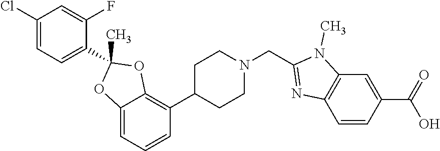 [SMILES: Cn1c(CN2CCC(c3cccc4c3O[C@@](C)(c3ccc(Cl)cc3F)O4)CC2)nc2ccc(C(=O)O)cc21] | 2-({4-[(2S)-2-(4-Chloro-2-fluorophenyl)-2- methyl-1,3-benzodioxol-4-yl]piperidin-1- yl}methyl)-1-methyl-1H-benzimidazole-6- carboxylic acid |
| 19B | 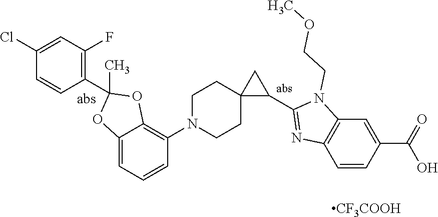 [SMILES: COCCn1c(C2CC23CCN(c2cccc4c2OC(C)(c2ccc(Cl)cc2F)O4)CC3)nc2ccc(C(=O)O)cc21 ; "·CF3COOH"] | 2-{6-[2-(4-Chloro-2-fluorophenyl)-2-methyl- 1,3-benzodioxol-4-yl]-6-azaspiro[2.5]oct-1- yl}-1-(2-methoxyethyl)-1H-benzimidazole- 6-carboxylic acid, DIAST-X1, trifluoroacetate salt |
| | DIAST-X1 | |
| 20B | 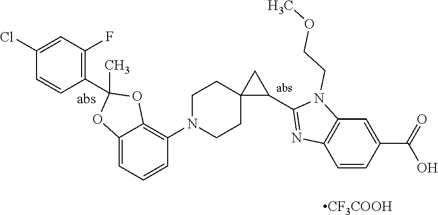 [SMILES: COCCn1c(C2CC23CCN(c2cccc4c2OC(C)(c2ccc(Cl)cc2F)O4)CC3)nc2ccc(C(=O)O)cc21 ; "·CF3COOH"] | 2-{6-[2-(4-Chloro-2-fluorophenyl)-2-methyl- 1,3-benzodioxol-4-yl]-6-azaspiro[2.5]oct-1- yl}-1-(2-methoxyethyl)-1H-benzimidazole- 6-carboxylic acid, DIAST-X2, trifluoroacetate salt |
| | DIAST-X2 | |
| 21B | 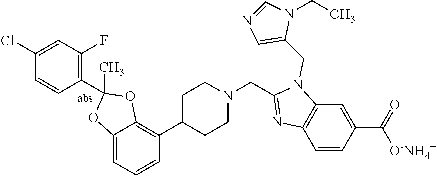 | Ammonium 2-({4-[2-(4-chloro-2- fluorophenyl)-2-methyl-1,3-benzodioxol-4- yl]piperidin-1-yl}methyl)-1-[(1-ethyl-1H- imidazol-5-yl)methyl]-1H-benzimidazole-6- carboxylate, ENT-1 |
| | ENT-1 | |
| 22B | 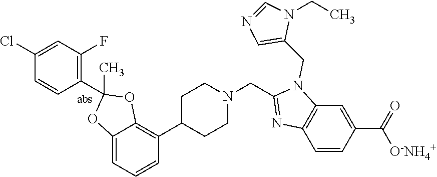 | Ammonium 2-({4-[2-(4-chloro-2- fluorophenyl)-2-methyl-1,3-benzodioxol-4- yl]piperidin-1-yl}methyl)-1-[(1-ethyl-1H- imidazol-5-yl)methyl]-1H-benzimidazole-6- carboxylate, ENT-2 |
| | ENT-2 | |
| 23B | 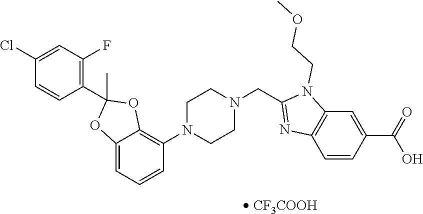 [SMILES: COCCn1c(CN2CCN(c3cccc4c3OC(C)(c3ccc(Cl)cc3F)O4)CC2)nc2ccc(C(=O)O)cc21 ; "· CF3COOH"] | 2-({4-[2-(4-chloro-2-fluorophenyl)-2-methyl- 1,3-benzodioxol-4-yl]piperazin-1- yl}methyl)-1-(2-methoxyethyl)-1H- benzimidazole-6-carboxylic acid, trifluoroacetate salt |
| 24B | 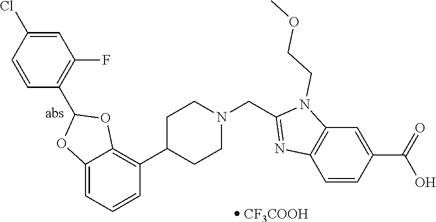 [SMILES: COCCn1c(CN2CCC(c3cccc4c3OC(c3ccc(Cl)cc3F)O4)CC2)nc2ccc(C(=O)O)cc21 ; "· CF3COOH"] | 2-({4-[2-(4-chloro-2-fluorophenyl)-1,3- benzodioxol-4-yl]piperidin-1-yl}methyl)-1- (2-methoxyethyl)-1H-benzimidazole-6- carboxylic acid, ENT-X2, trifluoroacetate salt |
| | ENT-X2 | |
| 25B | 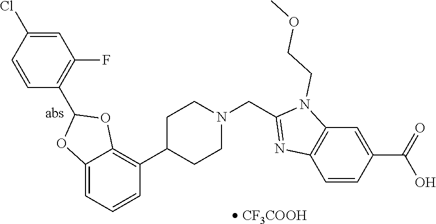 [SMILES: COCCn1c(CN2CCC(c3cccc4c3OC(c3ccc(Cl)cc3F)O4)CC2)nc2ccc(C(=O)O)cc21 ; "· CF3COOH"] | 2-({4-[2-(4-chloro-2-fluorophenyl)-1,3- benzodioxol-4-yl]piperidin-1-yl}methyl)-1- (2-methoxyethyl)-1H-benzimidazole-6- carboxylic acid, ENT-X1, trifluoroacetate salt |
| | ENT-X1 | |
| 26B | 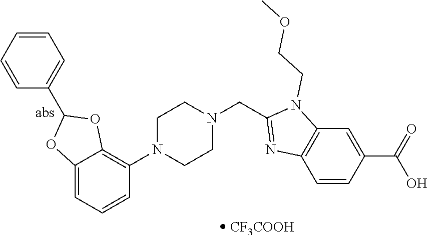 [SMILES: COCCn1c(CN2CCN(c3cccc4c3OC(c3ccccc3)O4)CC2)nc2ccc(C(=O)O)cc21 ; "· CF3COOH"] | 1-(2-methoxyethyl)-2-{[4-(2-phenyl-1,3- benzodioxol-4-yl)piperazin-1-yl]methyl}- 1H-benzimidazole-6-carboxylic acid, ENT- X1, trifluoroacetate salt |
| | ENT-X1 | |
| 27B | 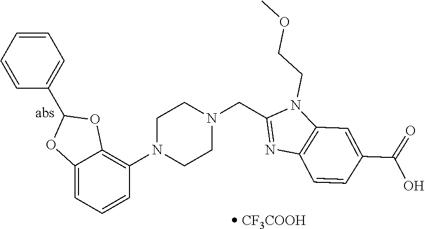 [SMILES: COCCn1c(CN2CCN(c3cccc4c3OC(c3ccccc3)O4)CC2)nc2ccc(C(=O)O)cc21 ; "· CF3COOH"] | 1-(2-methoxyethyl)-2-{[4-(2-phenyl-1,3- benzodioxol-4-yl)piperazin-1-yl]methyl}- 1H-benzimidazole-6-carboxylic acid, ENT- X2, trifluoroacetate salt |
| | ENT-X2 | |
| 28B | 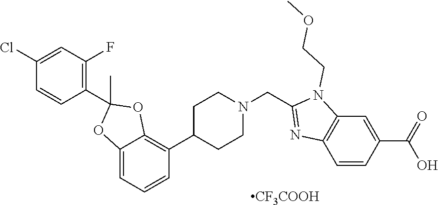 [SMILES: COCCn1c(CN2CCC(c3cccc4c3OC(C)(c3ccc(Cl)cc3F)O4)CC2)nc2ccc(C(=O)O)cc21 ; "·CF3COOH"] | 2-({4-[2-(4-chloro-2-fluorophenyl)-2-methyl- 1,3-benzodioxol-4-yl]piperidin-1-yl}methyl)- 1-(2-methoxyethyl)-1H-benzimidazole-6- carboxylic acid, trifluoroacetate salt |
| 29B | 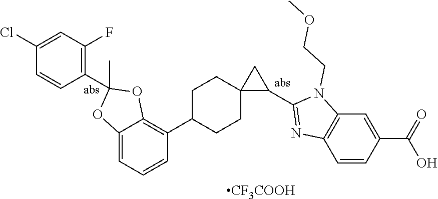 [SMILES: COCCn1c(C2CC23CCC(c2cccc4c2OC(C)(c2ccc(Cl)cc2F)O4)CC3)nc2ccc(C(=O)O)cc21 ; "·CF3COOH"] | 2-{6-[2-(4-chloro-2-fluorophenyl)-2-methyl- 1,3-benzodioxol-4-yl]-6-azaspiro[2.5]oct-1- yl}-1-(2-methoxyethyl)-1H-benzimidazole- 6-carboxylic acid, DIAST-Z2, trifluoroacetate salt |
| | DIAST-Z2 | |
| 30B | 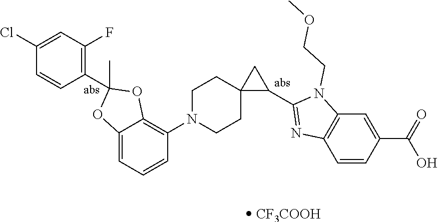 [SMILES: COCCn1c(C2CC23CCN(c2cccc4c2OC(C)(c2ccc(Cl)cc2F)O4)CC3)nc2ccc(C(=O)O)cc21 ; "· CF3COOH"] | 2-{6-[2-(4-chloro-2-fluorophenyl)-2-methyl- 1,3-benzodioxol-4-yl]-6-azaspiro[2.5]oct-1- yl}-1-(2-methoxyethyl)-1H-benzimidazole- 6-carboxylic acid, DIAST-Z1, trifluoroacetate salt |
| | DIAST-Z1 | |
| 31B | 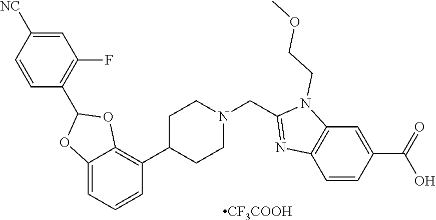 [SMILES: COCCn1c(CN2CCC(c3cccc4c3OC(c3ccc(C#N)cc3F)O4)CC2)nc2ccc(C(=O)O)cc21 ; "·CF3COOH"] | 2-({4-[2-(4-cyano-2-fluorophenyl)-1,3- benzodioxol-4-yl]piperidin-1-yl}methyl)-1- (2-methoxyethyl)-1H-benzimidazole-6- carboxylic acid, trifluoroacetate salt |
| 32B | 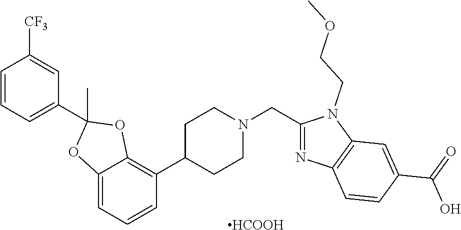 [SMILES: COCCn1c(CN2CCC(c3cccc4c3OC(C)(c3cccc(C(F)(F)F)c3)O4)CC2)nc2ccc(C(=O)O)cc21 ; "·HCOOH"] | 1-(2-methoxyethyl)-2-[(4-{2-methyl-2-[3- (trifluoromethyl)phenyl]-1,3-benzodioxol-4- yl}piperidin-1-yl)methyl]-1H-benzimidazole- 6-carboxylic acid, formate salt |
| 33B | 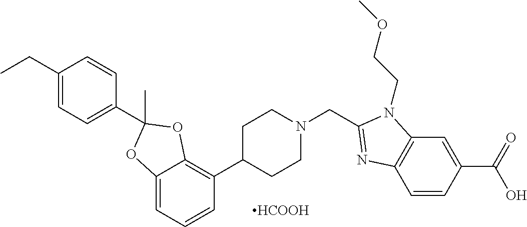 [SMILES: CCc1ccc(C2(C)Oc3cccc(C4CCN(Cc5nc6ccc(C(=O)O)cc6n5CCOC)CC4)c3O2)cc1 ; "·HCOOH"] | 2-({4-[2-(4-ethylphenyl)-2-methyl-1,3- benzodioxol-4-yl]piperidin-1-yl}methyl)-1- (2-methoxyethyl)-1H-benzimidazole-6- carboxylic acid, formate salt |
| 34B | 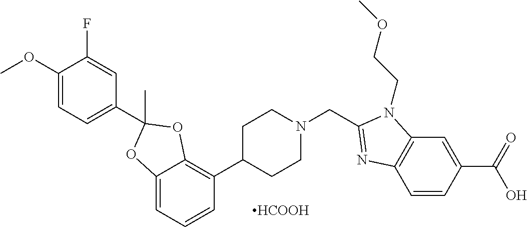 [SMILES: COCCn1c(CN2CCC(c3cccc4c3OC(C)(c3ccc(OC)c(F)c3)O4)CC2)nc2ccc(C(=O)O)cc21 ; "·HCOOH"] | 2-({4-[2-(3-fluoro-4-methoxyphenyl)-2- methyl-1,3-benzodioxol-4-yl]piperidin-1- yl}methyl)-1-(2-methoxyethyl)-1H- benzimidazole-6-carboxylic acid, formate salt |
| 35B | 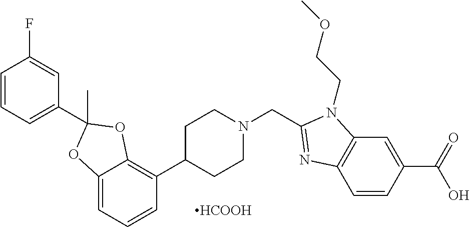 [SMILES: COCCn1c(CN2CCC(c3cccc4c3OC(C)(c3cccc(F)c3)O4)CC2)nc2ccc(C(=O)O)cc21 ; "·HCOOH"] | 2-({4-[2-(3-fluorophenyl)-2-methyl-1,3- benzodioxol-4-yl]piperidin-1-yl}methyl)-1- (2-methoxyethyl)-1H-benzimidazole-6- carboxylic acid, formate salt |
| 36B | 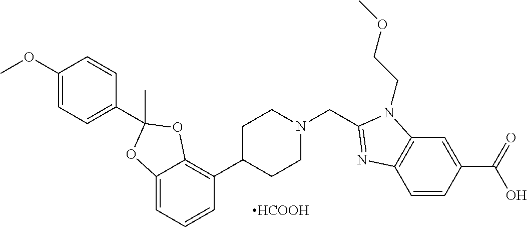 [SMILES: COCCn1c(CN2CCC(c3cccc4c3OC(C)(c3ccc(OC)cc3)O4)CC2)nc2ccc(C(=O)O)cc21 ; "·HCOOH"] | 1-(2-methoxyethyl)-2-({4-[2-(4- methoxyphenyl)-2-methyl-1,3-benzodioxol- 4-yl]piperidin-1-yl}methyl)-1H- benzimidazole-6-carboxylic acid, formate salt |
| 37B | 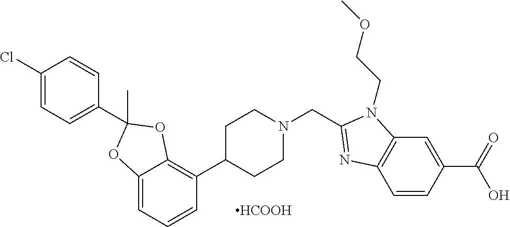 [SMILES: COCCn1c(CN2CCC(c3cccc4c3OC(C)(c3ccc(Cl)cc3)O4)CC2)nc2ccc(C(=O)O)cc21 ; "·HCOOH"] | 2-({4-[2-(4-chlorophenyl)-2-methyl-1,3- benzodioxol-4-yl]piperidin-1-yl}methyl)-1- (2-methoxyethyl)-1H-benzimidazole-6- carboxylic acid, formate salt |
| 38B | 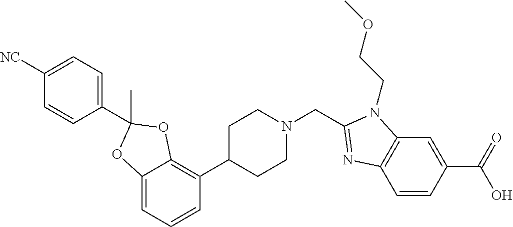 [SMILES: COCCn1c(CN2CCC(c3cccc4c3OC(C)(c3ccc(C#N)cc3)O4)CC2)nc2ccc(C(=O)O)cc21] | 2-({4-[2-(4-cyanophenyl)-2-methyl-1,3- benzodioxol-4-yl]piperidin-1-yl}methyl)-1- (2-methoxyethyl)-1H-benzimidazole-6- carboxylic acid |
| 39B | 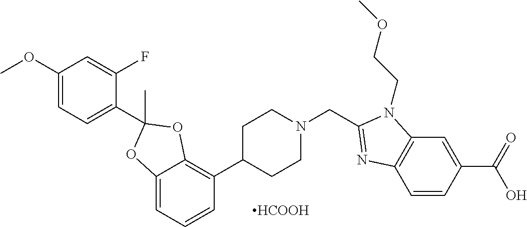 [SMILES: COCCn1c(CN2CCC(c3cccc4c3OC(C)(c3ccc(OC)cc3F)O4)CC2)nc2ccc(C(=O)O)cc21 ; "·HCOOH"] | 2-({4-[2-(2-fluoro-4-methoxyphenyl)-2- methyl-1,3-benzodioxol-4-yl]piperidin-1- yl}methyl)-1-(2-methoxyethyl)-1H- benzimidazole-6-carboxylic acid, formate salt |
| 40B | 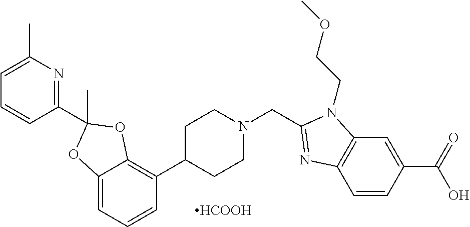 [SMILES: COCCn1c(CN2CCC(c3cccc4c3OC(C)(c3cccc(C)n3)O4)CC2)nc2ccc(C(=O)O)cc21 ; "·HCOOH"] | 1-(2-methoxyethyl)-2-({4-[2-methyl-2-(6- methylpyridin-2-yl)-1,3-benzodioxol-4- yl]piperidin-1-yl}methyl)-1H-benzimidazole- 6-carboxylic acid, formate salt |
| 41B | 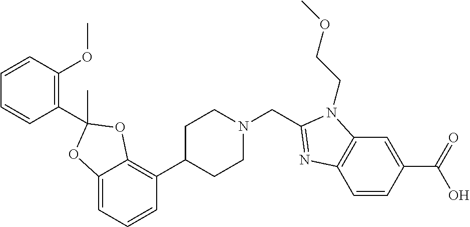 [SMILES: COCCn1c(CN2CCC(c3cccc4c3OC(C)(c3ccccc3OC)O4)CC2)nc2ccc(C(=O)O)cc21] | 1-(2-methoxyethyl)-2-({4-[2-(2- methoxyphenyl)-2-methyl-1,3-benzodioxol- 4-yl]piperidin-1-yl}methyl)-1H- benzimidazole-6-carboxylic acid |
| 42B | 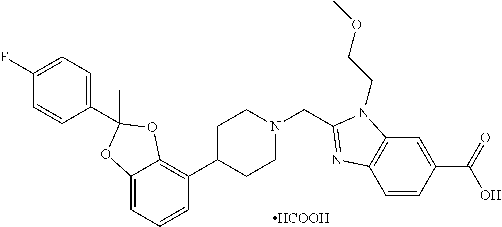 [SMILES: COCCn1c(CN2CCC(c3cccc4c3OC(C)(c3ccc(F)cc3)O4)CC2)nc2ccc(C(=O)O)cc21 ; "·HCOOH"] | 2-({4-[2-(4-fluorophenyl)-2-methyl-1,3- benzodioxol-4-yl]piperidin-1-yl}methyl)-1- (2-methoxyethyl)-1H-benzimidazole-6- carboxylic acid, formate salt |
| 43B | 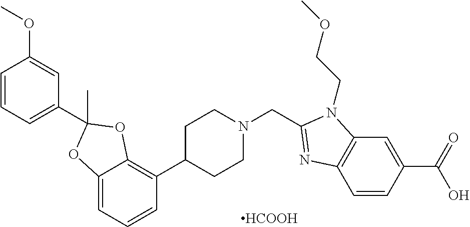 [SMILES: COCCn1c(CN2CCC(c3cccc4c3OC(C)(c3cccc(OC)c3)O4)CC2)nc2ccc(C(=O)O)cc21 ; "·HCOOH"] | 1-(2-methoxyethyl)-2-({4-[2-(3- methoxyphenyl)-2-methyl-1,3-benzodioxol- 4-yl]piperidin-1-yl}methyl)-1H- benzimidazole-6-carboxylic acid, formate salt |
| 44B | 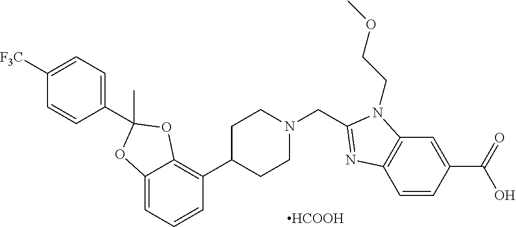 [SMILES: COCCn1c(CN2CCC(c3cccc4c3OC(C)(c3ccc(C(F)(F)F)cc3)O4)CC2)nc2ccc(C(=O)O)cc21 ; "·HCOOH"] | 1-(2-methoxyethyl)-2-[(4-{2-methyl-2-[4- (trifluoromethyl)phenyl]-1,3-benzodioxol-4- yl}piperidin-1-yl)methyl]-1H-benzimidazole- 6-carboxylic acid, formate salt |
| 45B | 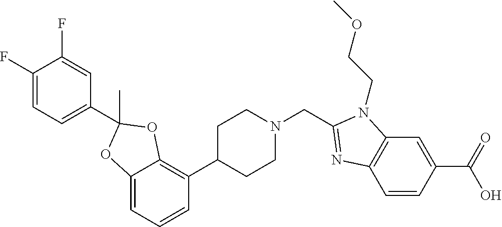 [SMILES: COCCn1c(CN2CCC(c3cccc4c3OC(C)(c3ccc(F)c(F)c3)O4)CC2)nc2ccc(C(=O)O)cc21] | 2-({4-[2-(3,4-difluorophenyl)-2-methyl-1,3- benzodioxol-4-yl]piperidin-1-yl}methyl)-1- (2-methoxyethyl)-1H-benzimidazole-6- carboxylic acid |
| 46B | 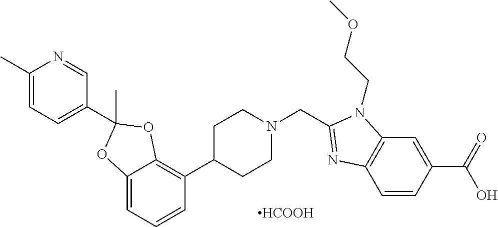 [SMILES: COCCn1c(CN2CCC(c3cccc4c3OC(C)(c3ccc(C)nc3)O4)CC2)nc2ccc(C(=O)O)cc21 ; "·HCOOH"] | 1-(2-methoxyethyl)-2-({4-[2-methyl-2-(6- methylpyridin-3-yl)-1,3-benzodioxol-4- yl]piperidin-1-yl}methyl)-1H-benzimidazole- 6-carboxylic acid, formate salt |
| 47B | 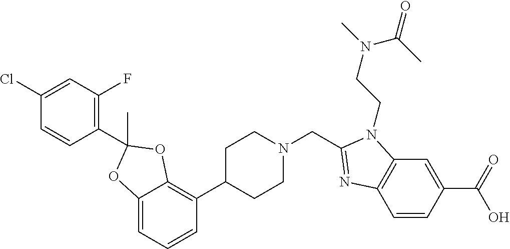 [SMILES: CC(=O)N(C)CCn1c(CN2CCC(c3cccc4c3OC(C)(c3ccc(Cl)cc3F)O4)CC2)nc2ccc(C(=O)O)cc21] | 1-{2-[acetyl(methyl)amino]ethyl}-2-({4-[2- (4-chloro-2-fluorophenyl)-2-methyl-1,3- benzodioxol-4-yl]piperidin-1-yl}methyl)-1H- benzimidazole-6-carboxylic acid |
| 48B | 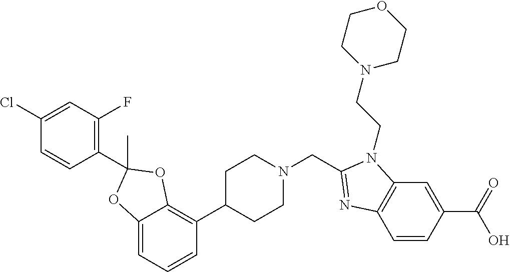 [SMILES: CC1(c2ccc(Cl)cc2F)Oc2cccc(C3CCN(Cc4nc5ccc(C(=O)O)cc5n4CCN4CCOCC4)CC3)c2O1] | 2-({4-[2-(4-chloro-2-fluorophenyl)-2-methyl- 1,3-benzodioxol-4-yl]piperidin-1-yl}methyl)- 1-[2-(morpholin-4-yl)ethyl]-1H- benzimidazole-6-carboxylic acid |
| 49B | 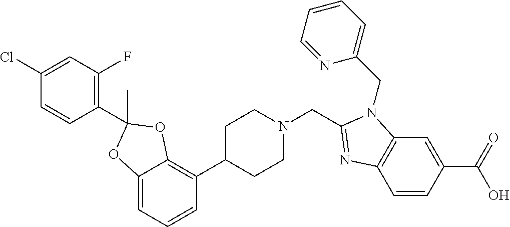 [SMILES: CC1(c2ccc(Cl)cc2F)Oc2cccc(C3CCN(Cc4nc5ccc(C(=O)O)cc5n4Cc4ccccn4)CC3)c2O1] | 2-({4-[2-(4-chloro-2-fluorophenyl)-2-methyl- 1,3-benzodioxol-4-yl]piperidin-1-yl}methyl)- 1-(pyridin-2-ylmethyl)-1H-benzimidazole-6- carboxylic acid |
| 50B | 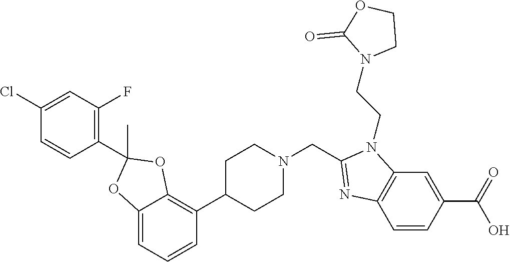 [SMILES: CC1(c2ccc(Cl)cc2F)Oc2cccc(C3CCN(Cc4nc5ccc(C(=O)O)cc5n4CCN4CCOC4=O)CC3)c2O1] | 2-({4-[2-(4-chloro-2-fluorophenyl)-2-methyl- 1,3-benzodioxol-4-yl]piperidin-1-yl}methyl)- 1-[2-(2-oxo-1,3-oxazolidin-3-yl)ethyl]-1H- benzimidazole-6-carboxylic acid |
| 51B | 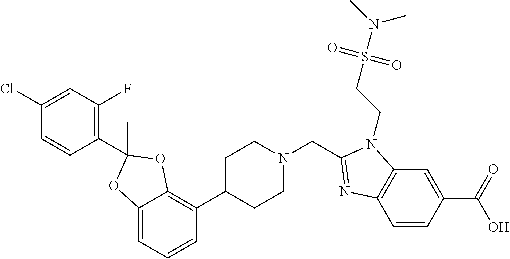 [SMILES: CN(C)S(=O)(=O)CCn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[2-(dimethylsulfamoyl)ethyl]-1H- benzimidazole-6-carboxylic acid |
| 52B | 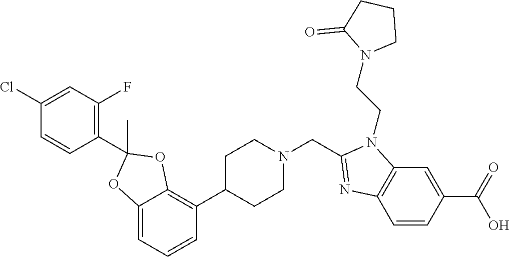 [SMILES: CC1(c2ccc(Cl)cc2F)Oc2cccc(C3CCN(Cc4nc5ccc(C(=O)O)cc5n4CCN4CCCC4=O)CC3)c2O1] | 2-({4-[2-(4-chloro-2-fluorophenyl)-2-methyl- 1,3-benzodioxol-4-yl]piperidin-1-yl}methyl)- 1-[2-(2-oxopyrrolidin-1-yl)ethyl]-1H- benzimidazole-6-carboxylic acid |
| 53B | 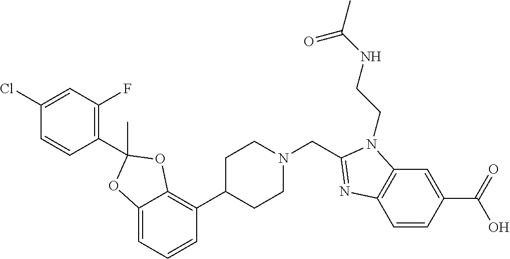 [SMILES: CC(=O)NCCn1c(CN2CCC(c3cccc4c3OC(C)(c3ccc(Cl)cc3F)O4)CC2)nc2ccc(C(=O)O)cc21] | 1-[2-(acetylamino)ethyl]-2-({4-[2-(4-chloro- 2-fluorophenyl)-2-methyl-1,3-benzodioxol- 4-yl]piperidin-1-yl}methyl)-1H- benzimidazole-6-carboxylic acid |
| 54B | 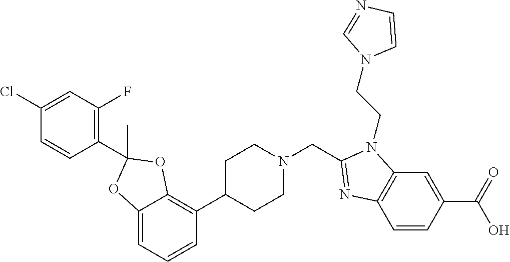 [SMILES: CC1(c2ccc(Cl)cc2F)Oc2cccc(C3CCN(Cc4nc5ccc(C(=O)O)cc5n4CCn4ccnc4)CC3)c2O1] | 2-({4-[2-(4-chloro-2-fluorophenyl)-2-methyl- 1,3-benzodioxol-4-yl]piperidin-1-yl}methyl)- 1-[2-(1H-imidazol-1-yl)ethyl]-1H- benzimidazole-6-carboxylic acid |
| 55B | 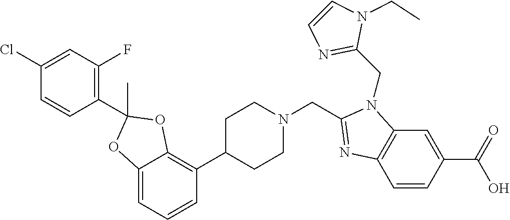 [SMILES: CCn1ccnc1Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[(1-ethyl-1H-imidazol-2-yl)methyl]-1H- benzimidazole-6-carboxylic acid |
| 56B | 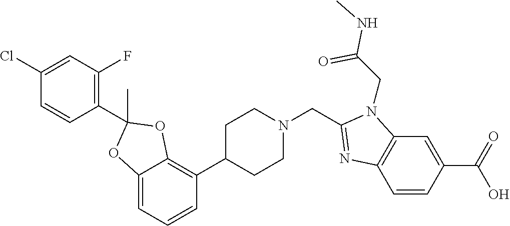 [SMILES: CNC(=O)Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[2-(methylamino)-2-oxoethyl]-1H- benzimidazole-6-carboxylic acid |
| 57B | 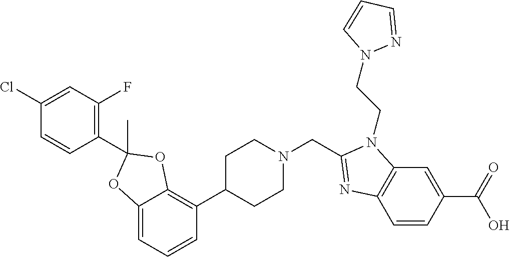 [SMILES: CC1(c2ccc(Cl)cc2F)Oc2cccc(C3CCN(Cc4nc5ccc(C(=O)O)cc5n4CCn4cccn4)CC3)c2O1] | 2-({4-[2-(4-chloro-2-fluorophenyl)-2-methyl- 1,3-benzodioxol-4-yl]piperidin-1-yl}methyl)- 1-[2-(1H-pyrazol-1-yl)ethyl]-1H- benzimidazole-6-carboxylic acid |
| 58B | 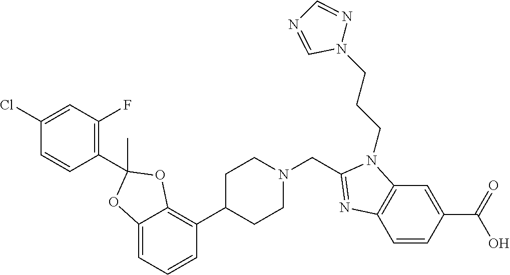 [SMILES: CC1(c2ccc(Cl)cc2F)Oc2cccc(C3CCN(Cc4nc5ccc(C(=O)O)cc5n4CCCn4cncn4)CC3)c2O1] | 2-({4-[2-(4-chloro-2-fluorophenyl)-2-methyl- 1,3-benzodioxol-4-yl]piperidin-1-yl}methyl)- 1-[3-(1H-1,2,4-triazol-1-yl)propyl]-1H- benzimidazole-6-carboxylic acid |
| 59B | 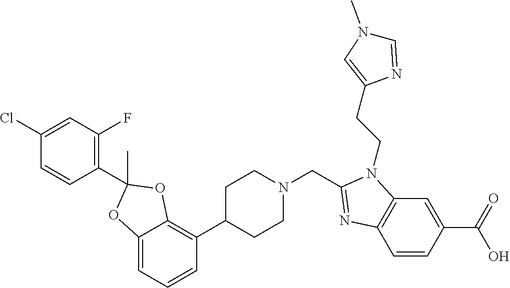 [SMILES: Cn1cnc(CCn2c(CN3CCC(c4cccc5c4OC(C)(c4ccc(Cl)cc4F)O5)CC3)nc3ccc(C(=O)O)cc32)c1] | 2-({4-[2-(4-chloro-2-fluorophenyl)-2-methyl- 1,3-benzodioxol-4-yl]piperidin-1-yl}methyl)- 1-[2-(1-methyl-1H-imidazol-4-yl)ethyl]-1H- benzimidazole-6-carboxylic acid |
| 60B | 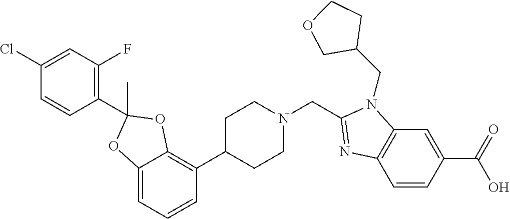 [SMILES: CC1(c2ccc(Cl)cc2F)Oc2cccc(C3CCN(Cc4nc5ccc(C(=O)O)cc5n4CC4CCOC4)CC3)c2O1] | 2-({4-[2-(4-chloro-2-fluorophenyl)-2-methyl- 1,3-benzodioxol-4-yl]piperidin-1-yl}methyl)- 1-(tetrahydrofuran-3-ylmethyl)-1H- benzimidazole-6-carboxylic acid |
| 61B | 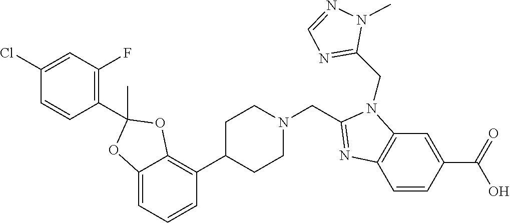 [SMILES: Cn1ncnc1Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[(1-methyl-1H-1,2,4-triazol-5-yl)methyl- 1H-benzimidazole-6-carboxylic acid |
| 62B | 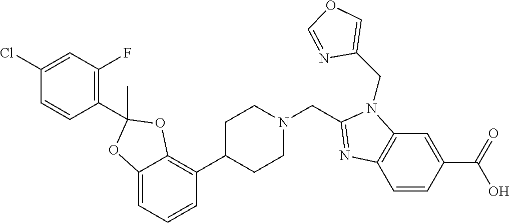 [SMILES: CC1(c2ccc(Cl)cc2F)Oc2cccc(C3CCN(Cc4nc5ccc(C(=O)O)cc5n4Cc4cocn4)CC3)c2O1] | 2-({4-[2-(4-chloro-2-fluorophenyl)-2-methyl- 1,3-benzodioxol-4-yl]piperidin-1-yl}methyl)- 1-(1,3-oxazol-4-ylmethyl)-1H- benzimidazole-6-carboxylic acid |
| 63B | 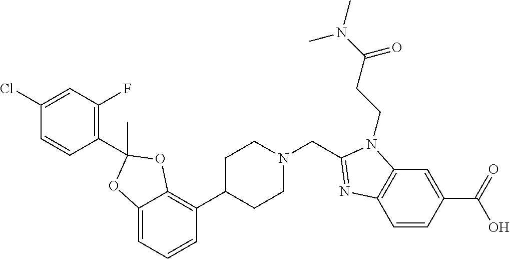 [SMILES: CN(C)C(=O)CCn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[3-(dimethylamino)-3-oxopropyl]-1H- benzimidazole-6-carboxylic acid |
| 64B | 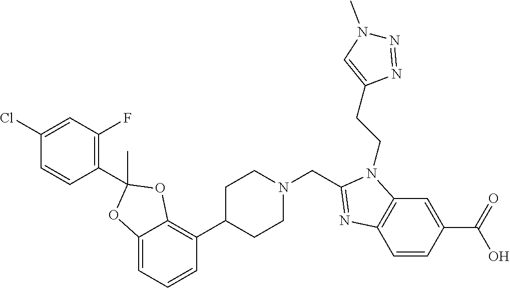 [SMILES: Cn1cc(CCn2c(CN3CCC(c4cccc5c4OC(C)(c4ccc(Cl)cc4F)O5)CC3)nc3ccc(C(=O)O)cc32)nn1] | 2-({4-[2-(4-chloro-2-fluorophenyl)-2-methyl- 1,3-benzodioxol-4-yl]piperidin-1-yl}methyl)- 1-[2-(1-methyl-1H-1,2,3-triazol-4-yl)ethyl]- 1H-benzimidazole-6-carboxylic acid |
| 65B | 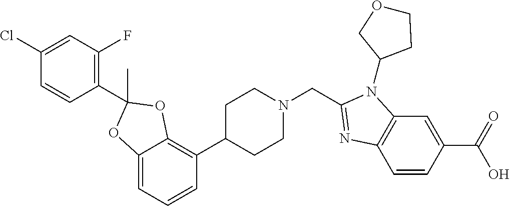 [SMILES: CC1(c2ccc(Cl)cc2F)Oc2cccc(C3CCN(Cc4nc5ccc(C(=O)O)cc5n4C4CCOC4)CC3)c2O1] | 2-({4-[2-(4-chloro-2-fluorophenyl)-2-methyl- 1,3-benzodioxol-4-yl]piperidin-1-yl}methyl)- 1-(tetrahydrofuran-3-yl)-1H-benzimidazole- 6-carboxylic acid |
| 66B | 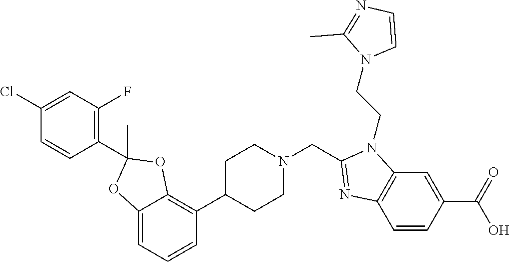 [SMILES: Cc1nccn1CCn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[2-(2-methyl-1H-imidazol-1-yl)ethyl]-1H- benzimidazole-6-carboxylic acid |
| 67B | 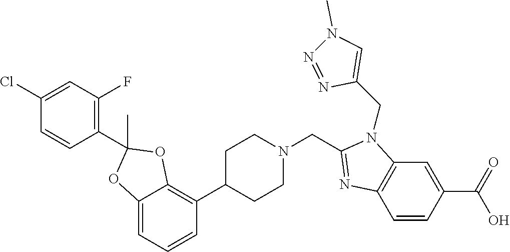 [SMILES: Cn1cc(Cn2c(CN3CCC(c4cccc5c4OC(C)(c4ccc(Cl)cc4F)O5)CC3)nc3ccc(C(=O)O)cc32)nn1] | 2-({4-[2-(4-chloro-2-fluorophenyl)-2-methyl- 1,3-benzodioxol-4-yl]piperidin-1-yl}methyl)- 1-[(1-methyl-1H-1,2,3-triazol-4-yl)methyl]- 1H-benzimidazole-6-carboxylic acid |
| 68B | 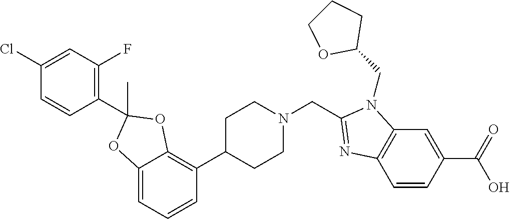 [SMILES: CC1(c2ccc(Cl)cc2F)Oc2cccc(C3CCN(Cc4nc5ccc(C(=O)O)cc5n4C[C@H]4CCCO4)CC3)c2O1] | 2-({4-[2-(4-chloro-2-fluorophenyl)-2-methyl- 1,3-benzodioxol-4-yl]piperidin-1-yl}methyl)- 1-[(2R)-tetrahydrofuran-2-ylmethyl]-1H- benzimidazole-6-carboxylic acid |
| 69B | 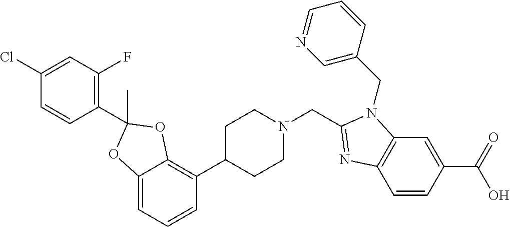 [SMILES: CC1(c2ccc(Cl)cc2F)Oc2cccc(C3CCN(Cc4nc5ccc(C(=O)O)cc5n4Cc4cccnc4)CC3)c2O1] | 2-({4-[2-(4-chloro-2-fluorophenyl)-2-methyl- 1,3-benzodioxol-4-yl]piperidin-1-yl}methyl)- 1-(pyridin-3-ylmethyl)-1H-benzimidazole-6- carboxylic acid |
| 70B | 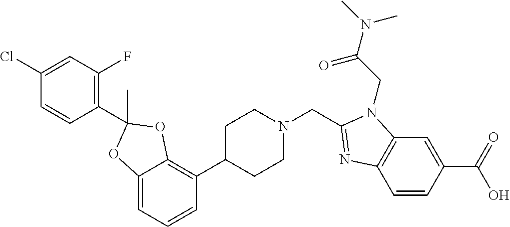 [SMILES: CN(C)C(=O)Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[2-(dimethylamino)-2-oxoethyl]-1H- benzimidazole-6-carboxylic acid |
| 71B | 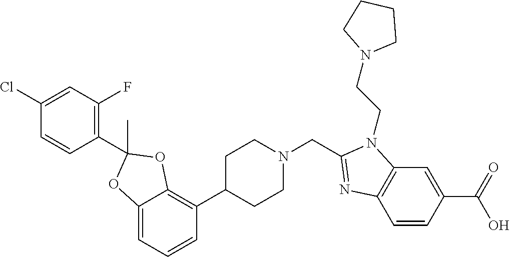 [SMILES: CC1(c2ccc(Cl)cc2F)Oc2cccc(C3CCN(Cc4nc5ccc(C(=O)O)cc5n4CCN4CCCC4)CC3)c2O1] | 2-({4-[2-(4-chloro-2-fluorophenyl)-2-methyl- 1,3-benzodioxol-4-yl]piperidin-1-yl}methyl)- 1-[2-(pyrrolidin-1-yl)ethyl]-1H- benzimidazole-6-carboxylic acid |
| 72B | 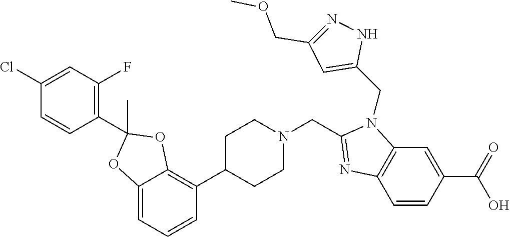 [SMILES: COCc1cc(Cn2c(CN3CCC(c4cccc5c4OC(C)(c4ccc(Cl)cc4F)O5)CC3)nc3ccc(C(=O)O)cc32)[nH]n1] | 2-({4-[2-(4-chloro-2-fluorophenyl)-2-methyl- 1,3-benzodioxol-4-yl]piperidin-1-yl}methyl)- 1-{[3-(methoxymethyl)-1H-pyrazol-5- yl]methyl}-1H- benzimidazole-6-carboxylic acid |
| 73B | 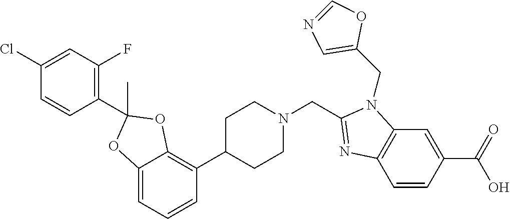 [SMILES: CC1(c2ccc(Cl)cc2F)Oc2cccc(C3CCN(Cc4nc5ccc(C(=O)O)cc5n4Cc4cnco4)CC3)c2O1] | 2-({4-[2-(4-chloro-2-fluorophenyl)-2-methyl- 1,3-benzodioxol-4-yl]piperidin-1-yl}methyl)- 1-(1,3-oxazol-5-ylmethyl)-1H- benzimidazole-6-carboxylic acid |
| 74B | 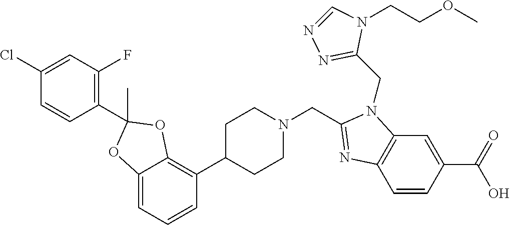 [SMILES: COCCn1cnnc1Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-{[4-(2-methoxyethyl)-4H-1,2,4-triazol-3- yl]methyl}-1H- benzimidazole-6-carboxylic acid |
| 75B | 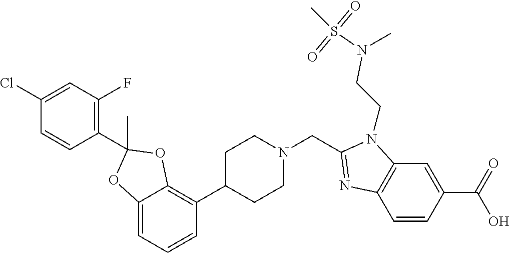 [SMILES: CN(CCn1c(CN2CCC(c3cccc4c3OC(C)(c3ccc(Cl)cc3F)O4)CC2)nc2ccc(C(=O)O)cc21)S(C)(=O)=O] | 2-({4-[2-(4-chloro-2-fluorophenyl)-2-methyl- 1,3-benzodioxol-4-yl]piperidin-1-yl}methyl)- 1-{2-[methyl(methylsulfonyl)amino]ethyl}- 1H-benzimidazole-6-carboxylic acid |
| 76B | 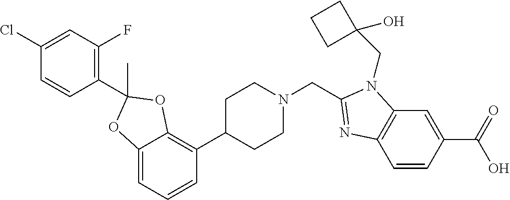 [SMILES: CC1(c2ccc(Cl)cc2F)Oc2cccc(C3CCN(Cc4nc5ccc(C(=O)O)cc5n4CC4(O)CCC4)CC3)c2O1] | 2-({4-[2-(4-chloro-2-fluorophenyl)-2-methyl- 1,3-benzodioxol-4-yl]piperidin-1-yl}methyl)- 1-[(1-hydroxycyclobutyl)methyl]-1H- benzimidazole-6-carboxylic acid |
| 77B | 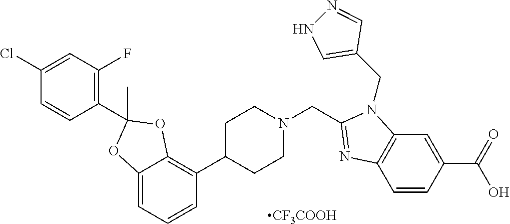 [SMILES: CC1(c2ccc(Cl)cc2F)Oc2cccc(C3CCN(Cc4nc5ccc(C(=O)O)cc5n4Cc4cn[nH]c4)CC3)c2O1 ; "·CF3COOH"] | 2-({4-[2-(4-chloro-2-fluorophenyl)-2-methyl- 1,3-benzodioxol-4-yl]piperidin-1-yl}methyl)- 1-(1H-pyrazol-4-ylmethyl)-1H- benzimidazole-6-carboxylic acid, trifluoroacetate salt |
| 78B | 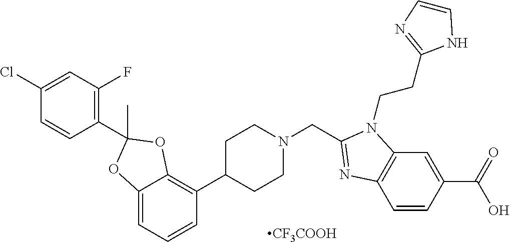 [SMILES: CC1(c2ccc(Cl)cc2F)Oc2cccc(C3CCN(Cc4nc5ccc(C(=O)O)cc5n4CCc4ncc[nH]4)CC3)c2O1 ; "·CF3COOH"] | 2-({4-[2-(4-chloro-2-fluorophenyl)-2-methyl- 1,3-benzodioxol-4-yl]piperidin-1-yl}methyl)- 1-[2-(1H-imidazol-2-yl)ethyl]-1H- benzimidazole-6-carboxylic acid, trifluoroacetate salt |
| 79B | 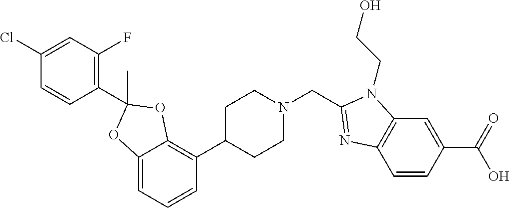 [SMILES: CC1(c2ccc(Cl)cc2F)Oc2cccc(C3CCN(Cc4nc5ccc(C(=O)O)cc5n4CCO)CC3)c2O1] | 2-({4-[2-(4-chloro-2-fluorophenyl)-2-methyl- 1,3-benzodioxol-4-yl]piperidin-1-yl}methyl)- 1-(2-hydroxyethyl)-1H-benzimidazole-6- carboxylic acid |
| 80B | 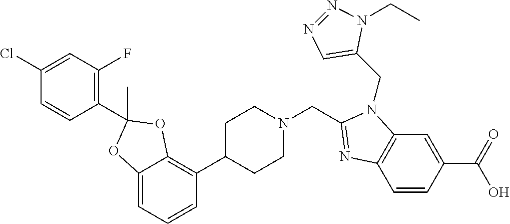 [SMILES: CCn1nncc1Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[(1-ethyl-1H-1,2,3-triazol-5-yl)methyl]- 1H-benzimidazole-6-carboxylic acid |
| 81B | 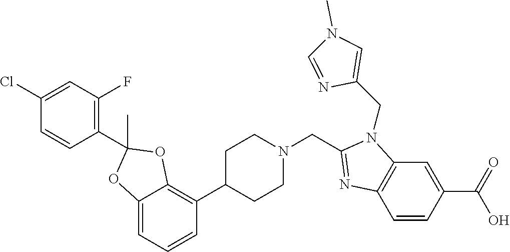 [SMILES: Cn1cnc(Cn2c(CN3CCC(c4cccc5c4OC(C)(c4ccc(Cl)cc4F)O5)CC3)nc3ccc(C(=O)O)cc32)c1] | 2-({4-[2-(4-chloro-2-fluorophenyl)-2-methyl- 1,3-benzodioxol-4-yl]piperidin-1-yl}methyl)- 1-[(1-methyl-1H-imidazol-4-yl)methyl]-1H- benzimidazole-6-carboxylic acid |
| 82B | 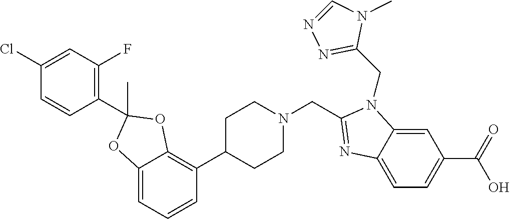 [SMILES: Cn1cnnc1Cn1c(CN2CCC(c3cccc4c3OC(C)(c3ccc(Cl)cc3F)O4)CC2)nc2ccc(C(=O)O)cc21] | 2-({4-[2-(4-chloro-2-fluorophenyl)-2-methyl- 1,3-benzodioxol-4-yl]piperidin-1-yl}methyl)- 1-[(4-methyl-4H-1,2,4-triazol-3-yl)methyl]- 1H-benzimidazole-6-carboxylic acid |
| 83B | 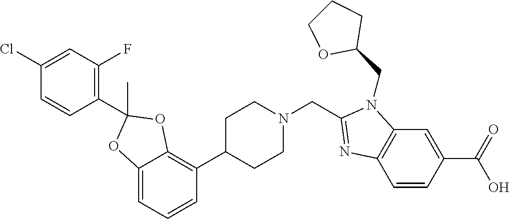 [SMILES: CC1(c2ccc(Cl)cc2F)Oc2cccc(C3CCN(Cc4nc5ccc(C(=O)O)cc5n4C[C@@H]4CCCO4)CC3)c2O1] | 2-({4-[2-(4-chloro-2-fluorophenyl)-2-methyl- 1,3-benzodioxol-4-yl]piperidin-1-yl}methyl)- 1-[(2S)-tetrahydrofuran-2-ylmethyl]-1H- benzimidazole-6-carboxylic acid |
| 84B | 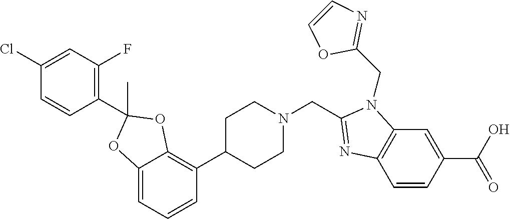 [SMILES: CC1(c2ccc(Cl)cc2F)Oc2cccc(C3CCN(Cc4nc5ccc(C(=O)O)cc5n4Cc4ncco4)CC3)c2O1] | 2-({4-[2-(4-chloro-2-fluorophenyl)-2-methyl- 1,3-benzodioxol-4-yl]piperidin-1-yl}methyl)- 1-(1,3-oxazol-2-ylmethyl)-1H- benzimidazole-6-carboxylic acid |
| 85B | 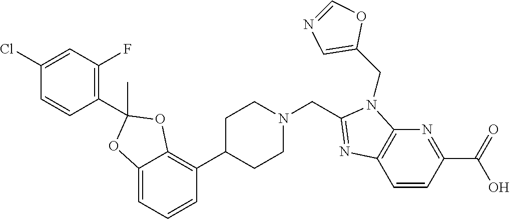 [SMILES: CC1(c2ccc(Cl)cc2F)Oc2cccc(C3CCN(Cc4nc5ccc(C(=O)O)nc5n4Cc4cnco4)CC3)c2O1] | 2-({4-[2-(4-chloro-2-fluorophenyl)-2-methyl- 1,3-benzodioxol-4-yl]piperidin-1-yl}methyl)- 3-(1,3-oxazol-5-ylmethyl)-3H-imidazo[4,5- b]pyridine-5-carboxylic acid |
| 86B | 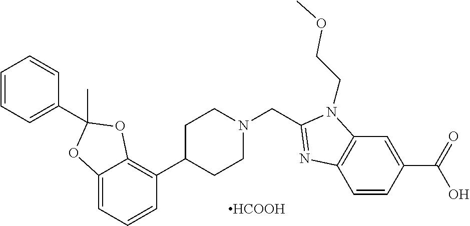 [SMILES: COCCn1c(CN2CCC(c3cccc4c3OC(C)(c3ccccc3)O4)CC2)nc2ccc(C(=O)O)cc21 ; "·HCOOH"] | 1-(2-methoxyethyl)-2-{[4-(2-methyl-2- phenyl-1,3-benzodioxol-4-yl)piperidin-1- yl]methyl}-1H-benzimidazole-6-carboxylic acid, formate salt |
| 87B | 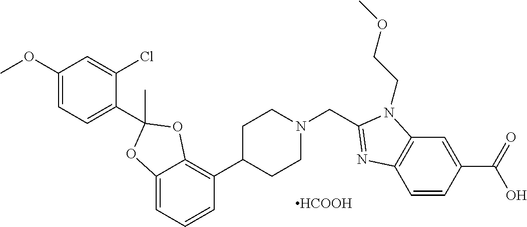 [SMILES: COCCn1c(CN2CCC(c3cccc4c3OC(C)(c3ccc(OC)cc3Cl)O4)CC2)nc2ccc(C(=O)O)cc21 ; "·HCOOH"] | 2-({4-[2-(2-chloro-4-methoxyphenyl)-2- methyl-1,3-benzodioxol-4-yl]piperidin-1- yl}methyl)-1-(2-methoxyethyl)-1H- benzimidazole-6-carboxylic acid, formate salt |
| 88B | 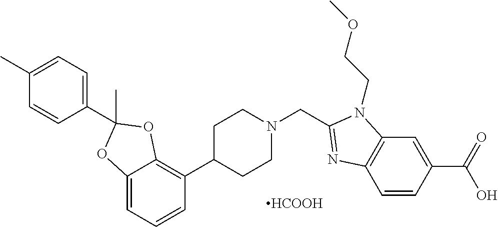 [SMILES: COCCn1c(CN2CCC(c3cccc4c3OC(C)(c3ccc(C)cc3)O4)CC2)nc2ccc(C(=O)O)cc21 ; "·HCOOH"] | 1-(2-methoxyethyl)-2-({4-[2-methyl-2-(4- methylphenyl)-1,3-benzodioxol-4- yl]piperidin-1-yl}methyl)-1H-benzimidazole- 6-carboxylic acid, formate salt |
| 89B | 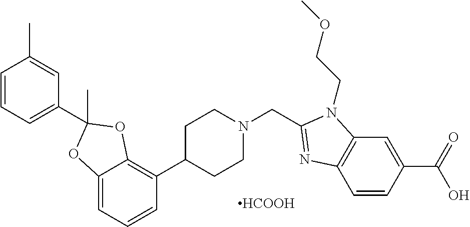 [SMILES: COCCn1c(CN2CCC(c3cccc4c3OC(C)(c3cccc(C)c3)O4)CC2)nc2ccc(C(=O)O)cc21 ; "·HCOOH"] | 1-(2-methoxyethyl)-2-({4-[2-methyl-2-(3- methylphenyl)-1,3-benzodioxol-4- yl]piperidin-1-yl}methyl)-1H-benzimidazole- 6-carboxylic acid, formate salt |
| 90B | 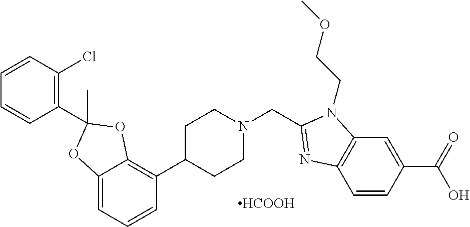 [SMILES: COCCn1c(CN2CCC(c3cccc4c3OC(C)(c3ccccc3Cl)O4)CC2)nc2ccc(C(=O)O)cc21 ; "·HCOOH"] | 2-({4-[2-(2-chlorophenyl)-2-methyl-1,3- benzodioxol-4-yl]piperidin-1-yl}methyl)-1- (2-methoxyethyl)-1H- benzimidazole-6-carboxylic acid, formate salt |
| 91B | 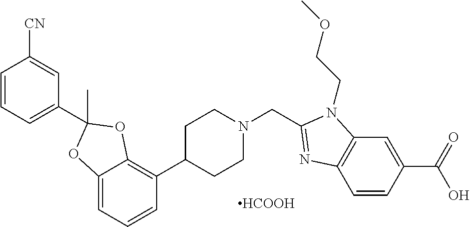 [SMILES: COCCn1c(CN2CCC(c3cccc4c3OC(C)(c3cccc(C#N)c3)O4)CC2)nc2ccc(C(=O)O)cc21 ; "·HCOOH"] | 2-({4-[2-(3-cyanophenyl)-2-methyl-1,3- benzodioxol-4-yl]piperidin-1-yl}methyl)-1- (2-methoxyethyl)-1H- benzimidazole-6-carboxylic acid, formate salt |
| 92B | 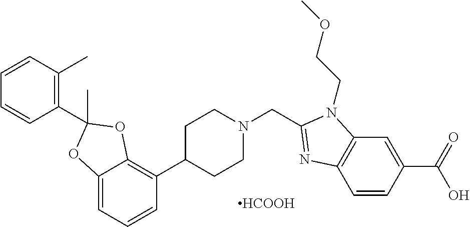 [SMILES: COCCn1c(CN2CCC(c3cccc4c3OC(C)(c3ccccc3C)O4)CC2)nc2ccc(C(=O)O)cc21 ; "·HCOOH"] | 1-(2-methoxyethyl)-2-({4-[2-methyl-2-(2- methylphenyl)-1,3-benzodioxol-4- yl]piperidin-1-yl}methyl)-1H-benzimidazole- 6-carboxylic acid, formate salt |
| 93B | 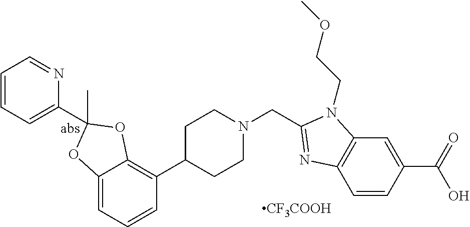 | 1-(2-methoxyethyl)-2-({4-[2-methyl-2- (pyridin-2-yl)-1,3-benzodioxol-4- yl]piperidin-1-yl}methyl)-1H-benzimidazole- 6-carboxylic acid, ENT-X2, trifluoroacetate salt |
| | ENT-X2 | |
| 94B | 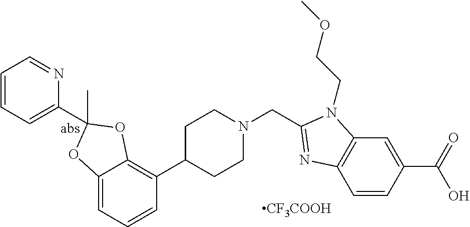 | 1-(2-methoxyethyl)-2-({4-[2-methyl-2- (pyridin-2-yl)-1,3-benzodioxol-4- yl]piperidin-1-yl}methyl)-1H- benzoimidazole-6-carboxylic acid, ENT-X1, trifluoroacetate salt |
| | ENT-X1 | |
| 95B | 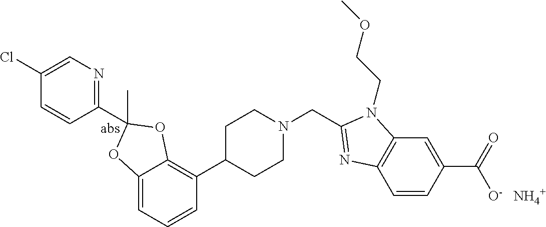 [SMILES: COCCn1c(CN2CCC(c3cccc4c3OC(C)(c3ccc(Cl)cn3)O4)CC2)nc2ccc(C(=O)[O-])cc21 ; [NH4+]] | ammonium 2-({4-[2-(5-chloropyridin-2-yl)- 2-methyl-1,3-benzodioxol-4-yl]piperidin-1- yl}methyl)-1-(2-methoxyethyl)-1H- benzimidazole-6-carboxylate, ENT-X1 |
| | ENT-X1 | |
| 96B | 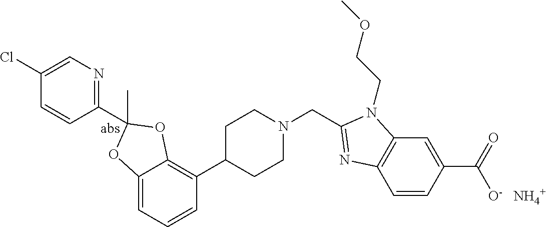 [SMILES: COCCn1c(CN2CCC(c3cccc4c3OC(C)(c3ccc(Cl)cn3)O4)CC2)nc2ccc(C(=O)[O-])cc21 ; [NH4+]] | ammonium 2-({4-[2-(5-chloropyridin-2-yl)- 2-methyl-1,3-benzodioxol-4-yl]piperidin-1- yl}methyl)-1-(2-methoxyethyl)-1H- benzimidazole-6-carboxylate, ENT-X2 |
| | ENT-X2 | |
| 97B | 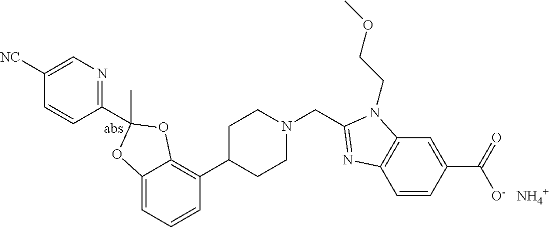 [SMILES: COCCn1c(CN2CCC(c3cccc4c3OC(C)(c3ccc(C#N)cn3)O4)CC2)nc2ccc(C(=O)[O-])cc21 ; [NH4+]] | ammonium 2-({4-[2-(5-cyanopyridin-2-yl)- 2-methyl-1,3-benzodioxol-4-yl]piperidin-1- yl}methyl)-1-(2-methoxyethyl)-1H- benzimidazole-6-carboxylate, ENT-X1 |
| | ENT-X1 | |
| 98B | 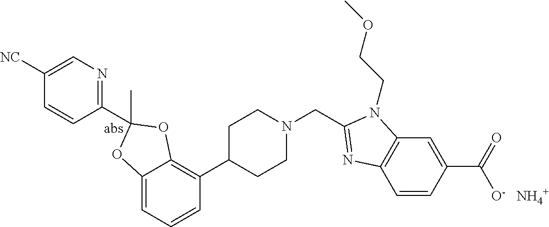 [SMILES: COCCn1c(CN2CCC(c3cccc4c3OC(C)(c3ccc(C#N)cn3)O4)CC2)nc2ccc(C(=O)[O-])cc21 ; [NH4+]] | ammonium 2-({4-[2-(5-cyanopyridin-2-yl)- 2-methyl-1,3-benzodioxol-4-yl]piperidin-1- yl}methyl)-1-(2-methoxyethyl)-1H- benzimidazole-6-carboxylate, ENT-X2 |
| | ENT-X2 | |
| 99B | 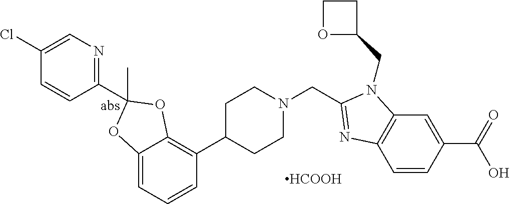 [SMILES: CC1(c2ccc(Cl)cn2)Oc2cccc(C3CCN(Cc4nc5ccc(C(=O)O)cc5n4C[C@@H]4CCO4)CC3)c2O1 ; "·HCOOH"] | 2-({4-[2-(5-chloropyridin-2-yl)-2-methyl-1,3- benzodioxol-4-yl]piperidin-1-yl}methyl)-1- [(2S)-oxetan-2-ylmethyl]-1H- benzimidazole-6-carboxylic acid, DIAST- X1 |
| | DIAST-X1 | |
| 100B | 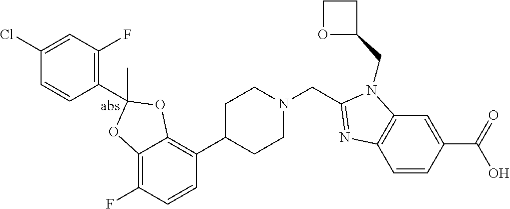 [SMILES: CC1(c2ccc(Cl)cc2F)Oc2c(F)ccc(C3CCN(Cc4nc5ccc(C(=O)O)cc5n4C[C@@H]4CCO4)CC3)c2O1] | 2-({4-[2-(4-chloro-2-fluorophenyl)-7-fluoro- 2-methyl-1,3-benzodioxol-4-yl]piperidin-1- yl}methyl)-1-[(2S)-oxetan-2-ylmethyl]-1H- benzimidazole-6-carboxylic acid, DIAST-1 |
| | DIAST-1 | |
| 101B | 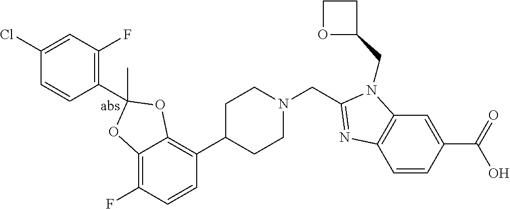 [SMILES: CC1(c2ccc(Cl)cc2F)Oc2c(F)ccc(C3CCN(Cc4nc5ccc(C(=O)O)cc5n4C[C@@H]4CCO4)CC3)c2O1] | 2-({4-[2-(4-chloro-2-fluorophenyl)-7-fluoro- 2-methyl-1,3-benzodioxol-4-yl]piperidin-1- yl}methyl)-1-[(2S)-oxetan-2-ylmethyl]-1H- benzimidazole-6-carboxylic acid, DIAST-2 |
| | DIAST-2 | |
| 102B | 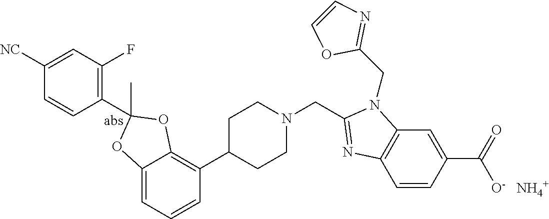 [SMILES: CC1(c2ccc(C#N)cc2F)Oc2cccc(C3CCN(Cc4nc5ccc(C(=O)[O-])cc5n4Cc4ncco4)CC3)c2O1 ; [NH4+]] | ammonium 2-({4-[2-(4-cyano-2- fluorophenyl)-2-methyl-1,3-benzodioxol-4- yl]piperidin-1-yl}methyl)-1-(1,3-oxazol-2- ylmethyl)-1H-benzimidazole-6-carboxylate, ENT-X2 |
| | ENT-X2 | |
| 103B | 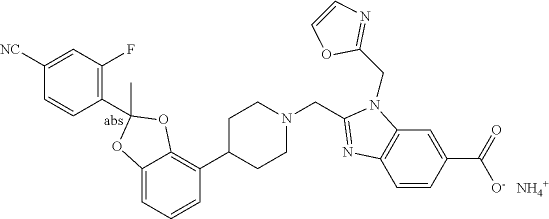 [SMILES: CC1(c2ccc(C#N)cc2F)Oc2cccc(C3CCN(Cc4nc5ccc(C(=O)[O-])cc5n4Cc4ncco4)CC3)c2O1 ; [NH4+]] | ammonium 2-({4-[2-(4-cyano-2- fluorophenyl)-2-methyl-1,3-benzodioxol-4- yl]piperidin-1-yl}methyl)-1-(1,3-oxazol-2- ylmethyl)-1H-benzimidazole-6-carboxylate, ENT-X1 |
| | ENT-X1 | |
| 104B | 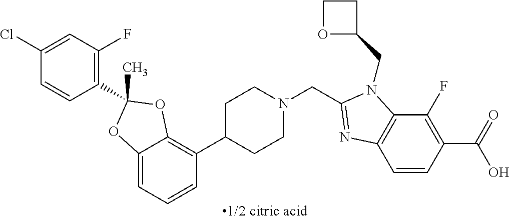 [SMILES: C[C@]1(c2ccc(Cl)cc2F)Oc2cccc(C3CCN(Cc4nc5ccc(C(=O)O)c(F)c5n4C[C@@H]4CCO4)CC3)c2O1] | 2-({4-[(2S)-2-(4-chloro-2-fluorophenyl)-2- methyl-1,3-benzodioxol-4-yl]piperidin-1- yl}methyl)-7-fluoro-1-[(2S)-oxetan-2- ylmethyl]-1H-benzimidazole-6-carboxylic acid, hemicitrate salt |
| 105B | 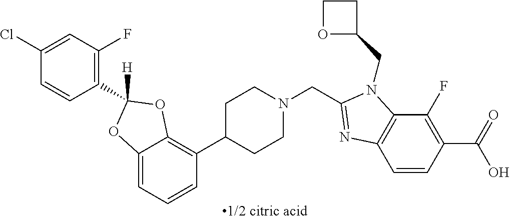 [SMILES: O=C(O)c1ccc2nc(CN3CCC(c4cccc5c4O[C@@H](c4ccc(Cl)cc4F)O5)CC3)n(C[C@@H]3CCO3)c2c1F] | 2-({4-[(2S)-2-(4-chloro-2-fluorophenyl)-1,3- benzodioxol-4-yl]piperidin-1-yl}methyl)-7- fluoro-1-[(2S)-oxetan-2-ylmethyl]-1H- benzimidazole-6-carboxylic acid, hemicitrate salt |
| 106B | 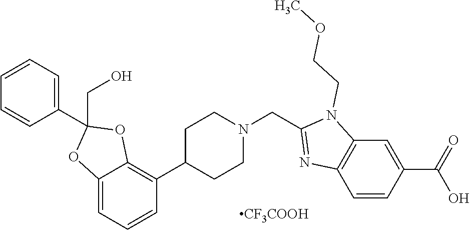 [SMILES: COCCn1c(CN2CCC(c3cccc4c3OC(CO)(c3ccccc3)O4)CC2)nc2ccc(C(=O)O)cc21 ; "·CF3COOH"] | 2-({4-[2-(hydroxymethyl)-2-phenyl-1,3- benzodioxol-4-yl]piperidin-1-yl}methyl)-1- (2-methoxyethyl)-1H-benzimidazole-6- carboxylic acid, trifluoroacetate salt |

The GLP1R agonist compound used in combination therapy with a GIPR antagonist compound disclosed herein can be a compound of Formula C-I:

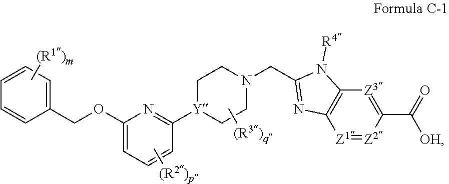

or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{5″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
   0 to 3 F atoms, and
   0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
each R^{Z″} is independently H, F, Cl, or —CH₃.

Another embodiment concerns compounds of Formula C-II

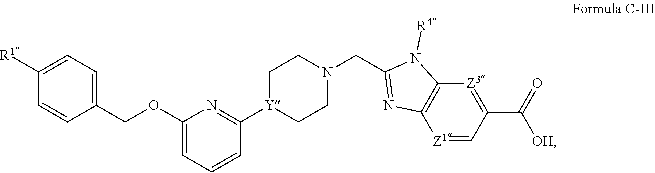

or a pharmaceutically acceptable salt thereof, wherein
m″ is 0 or 1;
R^{2″} is F;
p″ is 0, or 1; and
q″ is 0 or 1.

In some embodiments, disclosed herein is a compound of Formula C-I or Formula C-II, wherein:
m″ is 0 or 1;
q″ is 0 or 1; and
R^{3″} is —F, —CH₃, —CH₂CH₃, —CH₂OH, —CF₃, isopropyl, or cyclopropyl, or a pharmaceutically acceptable salt thereof.

A further embodiment concerns a compound of Formula C-III:

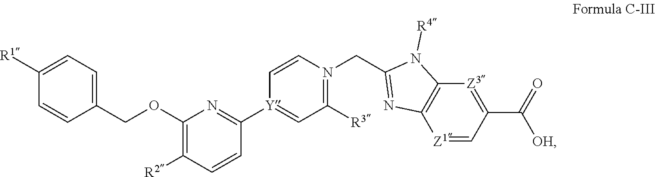

or a pharmaceutically acceptable salt thereof, wherein
m″ is 0 or 1;
R^{2″} is F;
p″ is 0, or 1;
R^{3″} is —C₁₋₂alkyl, wherein —C₁₋₂alkyl may be substituted as valency allows with 0 to 3 F atoms; and
q″ is 0 or 1.

In some embodiments, the compound has the Formula C-I, C-II, or C-III, wherein each R^{1″} is independently F, Cl, —CN, —CH₃, or —CF₃, or a pharmaceutically acceptable salt thereof. In some embodiments, the compound has the Formula C-I, C-II, or C-III, R^{3″} is —CH₃; q is 0 or 1; and R^{4″} is —CH₂CH₂OCH₃, C₁₋₃alkylene-R^{5″}, or C₁₋₃alkylene-R^{6″}. In some embodiments, the compound has the Formula C-I, C-II, or C-III, R^{4″} is —CH₂—R^{5″}, wherein R^{5″} is 4-membered or 5-membered heterocycloalkyl, wherein said heterocycloalkyl is substituted with 0 to 2 substituents as valency allows independently selected from 0 to 2 F atoms and 0 to 1 substituent selected from —OCH₃ and —CH₂OCH₃.

In some embodiments, the compound has the Formula C-I, C-II, or C-III, heterocycloalkyl is selected from the group consisting of:

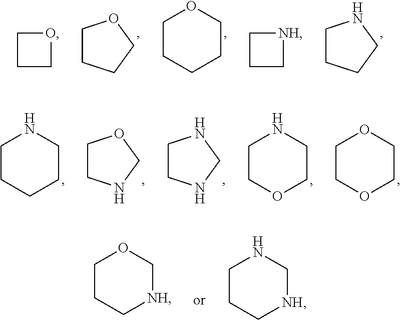

each of which is independently substituted with 0 to 2 substituents as valency allows, selected from the group consisting of: 0 to 1 oxo (0=), 0 to 1 —CN, 0 to 2 F atoms, and 0 to 2 substituents independently selected from —C₁₋₃alkyl and —OC₁₋₃alkyl, wherein the alkyl of C₁₋₃alkyl and OC₁₋₃alkyl may be independently substituted with 0 to 3 substituents as valency allows independently selected from: 0 to 3 F atoms, 0 to 1 —CN, and 0 to 1 —OR^{O″}.

Another embodiment concerns compounds of Formulas C-I, C-II, or C-III, wherein the heterocycloalkyl is

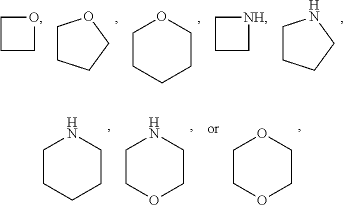

wherein the heterocycloalkyl may be substituted with 0 to 2 substituents as valency allows, e.g., replacing hydrogen, independently selected from:
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be independently substituted with 0 to 3 substituents as valency allows independently selected from:
   0 to 3 F atoms,
   0 to 1 —CN, and
   0 to 1 —OR^{O″}, or a pharmaceutically acceptable salt thereof.

Another embodiment concerns compounds of Formulas C-I, C-II, or C-III, wherein the heterocycloalkyl is

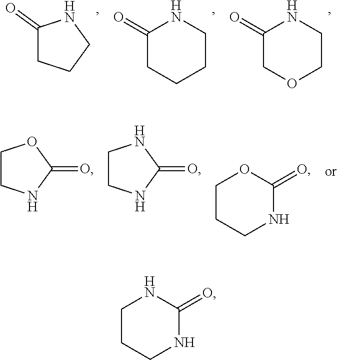

wherein the heterocycloalkyl may be substituted with 0 to 1 substituent as valency allows, e.g., replacing hydrogen, selected from:
—CN,
F atom, and
0 to 1 substituent independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O″}, or a pharmaceutically acceptable salt thereof.

Another embodiment concerns compounds of Formulas C-I, C-II, or C-III, wherein the heterocycloalkyl is

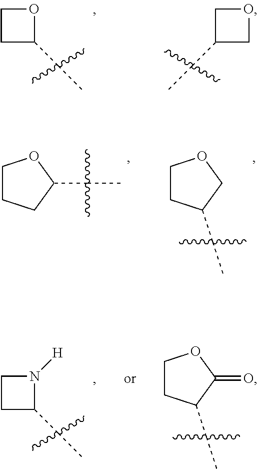

and wherein the heterocycloalkyl may be substituted with 0 to 1 substituent as valency allows, e.g., replacing hydrogen, selected from:
—CN,
F atom, and
0 to 1 substituent independently selected from —C₁₋₃alkyl and —OC₁₋₃alkyl, wherein the alkyl of C₁₋₃alkyl and OC₁₋₃alkyl may be substituted with 0 to 3 substituents as valency allows with:
   0 to 3 F atoms,
   0 to 1 —CN, or
   0 to 1 —OR^{O″}, or a pharmaceutically acceptable salt thereof.

Another embodiment concerns compounds of Formulas C-I, C-II, or C-III, wherein the heterocycloalkyl is

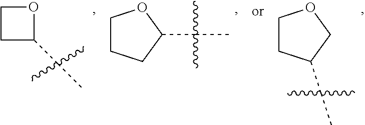 [SMILES: *[C@@H]1CCO1 ; *[C@@H]1CCCO1 ; *[C@@H]1CCOC1]

and wherein the heterocycloalkyl may be substituted as valency allows with 0 to 1 methyl, wherein said methyl may be substituted with 0 to 3 F atoms, or a pharmaceutically acceptable salt thereof.

Another embodiment concerns compounds of Formulas C-I, C-II, or C-III, wherein
R^{4″} is —CH₂—R^{6″}, wherein R^{6″} is the 5-membered heteroaryl, wherein said heteroaryl may be substituted with 0 to 2 substituents as valency allows independently selected from:
0 to 2 halogens, wherein the halogen is independently selected from F and Cl,
0 to 1 —OCH₃, and
0 to 1 —CH₃, —CH₂CH₃, —CF₃, or —CH₂CH₂OCH₃;
   or a pharmaceutically acceptable salt thereof.

Another embodiment concerns compounds of Formulas C-I, C-II, or C-III, wherein the heteroaryl is

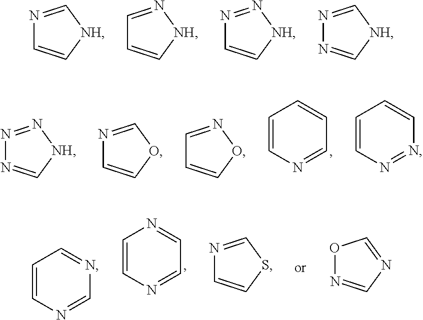

wherein said heteroaryl may be substituted with 0 to 2 substituents as valency allows, e.g., replacing hydrogen, independently selected from:
0 to 2 halogens, wherein the halogen is independently selected from F and Cl,
0 to 1 substituent selected from —OR^{O} and —N(R^{N})₂, or
0 to 2 —C₁₋₃alkyl, wherein the alkyl may be substituted with 0 to 3 substituents as valency allows independently selected from:
   0 to 3 F atoms, and
   0 to 1 —OR^{O″};
      or a pharmaceutically acceptable salt thereof.

Another embodiment concerns compounds of Formulas C-I, C-II, or C-III, wherein the heteroaryl is

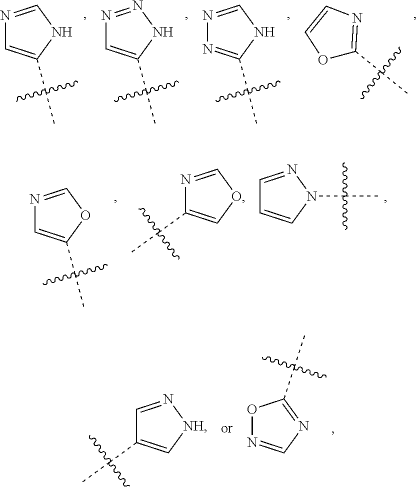

wherein said heteroaryl may be substituted with 0 to 2 substituents as valency allows, e.g., replacing hydrogen, independently selected from:
0 to 2 halogens, wherein the halogen is independently selected from F and Cl,
0 to 1 substituent selected from —OR^{O″} and —N(R^{N″})₂, or
0 to 2 —C₁₋₃alkyl, wherein the alkyl may be substituted with 0 to 3 substituents as valency allows independently selected from:
   0 to 3 F atoms, and
   0 to 1 —OR^{O″};
      or a pharmaceutically acceptable salt thereof.

Another embodiment concerns compounds of Formulas C-I, C-II, or C-III, wherein the heteroaryl is

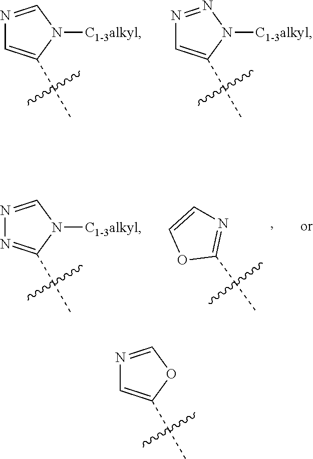

wherein C₁₋₃ alkyl on said heteroaryl may be substituted with 0 to 3 substituents as valency allows, e.g., replacing hydrogen, independently selected from:
0 to 3 F atoms, and
0 to 1 —OR^{O″};
   or a pharmaceutically acceptable salt thereof.

Another embodiment concerns compounds of other embodiments herein, e.g., compounds of Formulas C-I, C-II, or C-III, wherein Z^{1″}, Z^{2″}, and Z^{3″} are each CR^{Z″}, or a pharmaceutically acceptable salt thereof. Another embodiment concerns compounds of other embodiments herein, e.g., compounds of Formulas C-I, C-II, or C-III, wherein R^{Z″} is H, or a pharmaceutically acceptable salt thereof. Another embodiment concerns compounds of other embodiments herein, e.g., compounds of Formulas C-I, C-II, or C-III, wherein Z^{1″}, Z^{2″}, and Z^{3″} are each CH, or a pharmaceutically acceptable salt thereof.

Another embodiment concerns compounds of other embodiments herein, e.g., compounds of Formulas C-I, C-II, or C-III, wherein each R^{1″} is independently F, Cl, or —CN, or a pharmaceutically acceptable salt thereof.

Another embodiment concerns compounds of other embodiments herein, e.g., compounds of Formulas C-I, C-II, or C-III, wherein p″ is 0 or 1; and R^{2″} is F.

Another embodiment concerns compounds of other embodiments herein, e.g., compounds of Formulas C-I, C-II, or C-III, wherein R^{3″} is —CH₃, or —CF₃; and q″ is 1, or a pharmaceutically acceptable salt thereof.

Another embodiment concerns compounds of other embodiments herein, e.g., compounds of Formulas C-I, C-II, or C-III, wherein R^{4″} is —CH₂—R⁶, or a pharmaceutically acceptable salt thereof. Another embodiment concerns compounds of other embodiments herein, e.g., compounds of Formulas C-I, C-II, or C-III, wherein R^{4″} is —CH₂—R^{6″}, or a pharmaceutically acceptable salt thereof.

In some embodiments, the GLP-1R agonist is a compound selected from the group consisting of:
2-[(4-{6-[(4-chloro-2-fluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2R)-oxetan-2-ylmethyl]-1H-benzimidazole-6-carboxylic acid;
2-{[4-(6-{[(4-cyano-2-fluorophenyl)(methyl-d2)]oxy}pyridin-2-yl)piperidin-1-yl]methyl}-1-[(2S)-oxetan-2-ylmethyl]-1H-benzimidazole-6-carboxylic acid;
2-[(4-{6-[(4-cyano-2-fluorobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-chloro-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(2,4-difluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(2,4-difluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-chloro-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-chloro-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3S)-tetrahydrofuran-3-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2R)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2R)-tetrahydrofuran-2-ylmethyl]-1H-benzimidazole-6-carboxylic acid;
2-{[(2S)-4-{6-[(2,4-difluorobenzyl)oxy]-5-fluoropyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(2,4-difluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid; and
2-{[(2S)-4-{6-[(4-cyanobenzyl)oxy]pyridin-2-yl}-2-methylpiperazin-1-yl]methyl}-1-[(2S)-oxetan-2-ylmethyl]-1H-benzimidazole-6-carboxylic acid;
   or a pharmaceutically acceptable salt thereof.

In some embodiments, the GLP-1R agonist is 2-[(4-{6-[(4-chloro-2-fluorobenzyl)oxy]pyridin-2-yl}piperidin-1-yl)methyl]-1-[(2S)-oxetan-2-ylmethyl]-1H-benzimidazole-6-carboxylic acid, or a pharmaceutically acceptable salt thereof. In some embodiments, the GLP-1R agonist is 2-[(4-{6-[(4-cyano-2-fluorobenzyl)oxy]pyridin-2-yl}piperazin-1-yl)methyl]-1-[(2S)-oxetan-2-ylmethyl]-1H-benzimidazole-6-carboxylic acid, or a pharmaceutically acceptable salt thereof. In some embodiments, the GLP-1R agonist is 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid, or a pharmaceutically acceptable salt thereof.

In some embodiments, the GLP-1R agonist is a tris salt of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid. In some embodiments, the GLP-1R agonist is a free acid of 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid. In some embodiments, the GLP-1R agonist is 2-{[4-(6-{[(4-cyano-2-fluorophenyl)(methyl-d2)]oxy}pyridin-2-yl)piperidin-1-yl]methyl}-1-[(2S)-oxetan-2-ylmethyl]-1H-benzimidazole-6-carboxylic acid, or a pharmaceutically acceptable salt thereof. In some embodiments, the GLP-1R agonist is 2-[(4-{6-[(4-cyano-2-fluorobenzyl)oxy]-5-fluoropyridin-2-yl}piperidin-1-yl)methyl]-1-[(2S)-oxetan-2-ylmethyl]-1H-benzimidazole-6-carboxylic acid, or a pharmaceutically acceptable salt thereof.

In some embodiments, the GLP-1R agonist is a compound selected from the group consisting of:
2-[(4-{6-[(4-cyano-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-5-ylmethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-5-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-cyano-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-4-ylmethyl)-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,2-oxazol-5-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,2-oxazol-3-ylmethyl)-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid; and
2-[(4-{6-[(4-cyano-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
   or a pharmaceutically acceptable salt thereof.

In some embodiments, the GLP-1R agonist is a compound selected from the group consisting of:
2-[(4-{6-[(4-cyano-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-5-ylmethyl)-1H-benzimidazole-6-carboxylic acid; or
   2-[(4-{6-[(4-cyano-2-fluorobenzyl)oxy]pyridin-2-yl}piperazin-1-yl)methyl]-1-(1,3-oxazol-5-ylmethyl)-1H-benzimidazole-6-carboxylic acid;
      or a pharmaceutically acceptable salt thereof.

In some embodiments, the GLP-1R agonist is a compound selected from the group consisting of:
2-[(4-{6-[(4-cyanobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-cyanobenzyl)oxy]pyridin-2-yl}piperazin-1-yl)methyl]-1-(2-methoxyethyl)-1H-benzimidazole-6-carboxylic acid;
2-{[(2S)-4-{6-[(4-cyanobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-chloro-2-fluorobenzyl)oxy]-5-fluoropyridin-2-yl}-2-methylpiperazin-1-yl]methyl}-1-(2-methoxyethyl)-1H-benzimidazole-6-carboxylic acid;
2-{[(2S)-4-{6-[(4-cyanobenzyl)oxy]-5-fluoropyridin-2-yl}-2-methylpiperazin-1-yl]methyl}-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-1-(2-methoxyethyl)-1H-benzimidazole-6-carboxylic acid; or
   2-[(4-{6-[(4-chloro-2-fluorobenzyl)oxy]pyridin-2-yl}piperidin-1-yl)methyl]-1-[(1-methoxycyclobutyl)methyl]-1H-benzimidazole-6-carboxylic;
      or a pharmaceutically acceptable salt thereof.

The GLP-1R antagonist compounds of the disclosure are compounds comprising a molecular weight of from about 400 Da to about 700 Da, from about 400 Da to about 500 Da, from about 500 Da to about 600 Da, from about 600 Da to about 700 Da, from about 450 Da to about 550 Da, from about 400 Da to about 450 Da, from about 450 Da to about 500 Da, from about 500 Da to about 550 Da, from about 550 Da to about 600 Da, from about 600 Da to about 650 Da, and from about 650 Da to about 700 Da. In some embodiments, the GLP-1R antagonist compounds of the disclosure comprise a molecular weight of from about 450 Da to about 500 Da. In some embodiments, the GLP-1R antagonist compounds of the disclosure comprise a molecular weight of from about 500 Da to about 550 Da. In some embodiments, the GLP-1R antagonist compounds of the disclosure comprise a molecular weight of from about 550 Da to about 600 Da.

TABLE 3 shows additional GLP-1R agonists compounds of the disclosure that can be used in combination with the GIPR antagonist compounds disclosed herein.

TABLE 3
Additional GLP-1R agonist compounds for GIPR antagonist combination therapy

| GLP1R | | |
|---|---|---|
| Ag | Structure | IUPAC Name |
| 107B | 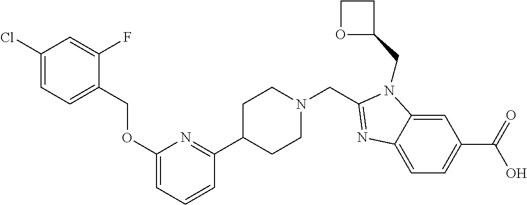 [SMILES: O=C(O)c1ccc2nc(CN3CCC(c4cccc(OCc5ccc(Cl)cc5F)n4)CC3)n(C[C@@H]3CCO3)c2c1] | 2-[(4-{6-[(4-chloro-2- fluorobenzyl)oxy]pyridin- 2-yl}piperidin-1-yl)methyl]- 1-[(2S)-oxetan-2-ylmethyl]-1H- benzimidazole-6-carboxylic acid |
| 108B | 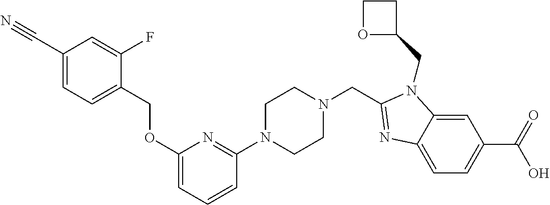 [SMILES: N#Cc1ccc(COc2cccc(N3CCN(Cc4nc5ccc(C(=O)O)cc5n4C[C@@H]4CCO4)CC3)n2)c(F)c1] | 2-[(4-{6-[(4-cyano-2- fluorobenzyl)oxy]pyridin- 2-yl}piperazin-1-yl)methyl]- 1-[(2S)-oxetan-2-ylmethyl]- 1H-benzimidazole-6- carboxylic acid |
| 109B | 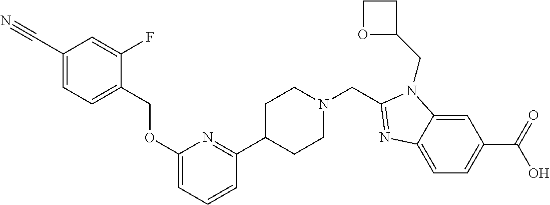 [SMILES: N#Cc1ccc(COc2cccc(C3CCN(Cc4nc5ccc(C(=O)O)cc5n4CC4CCO4)CC3)n2)c(F)c1] | 2-[(4-{6-[(4-cyano-2- fluorobenzyl)oxy]pyridin- 2-yl}piperidin-1-yl)methyl]- 1-[oxetan-2-ylmethyl]-1H- benzimidazole-6- carboxylic acid |
| 110B | 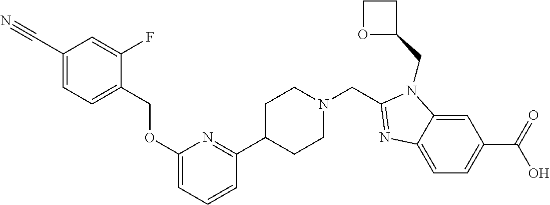 [SMILES: N#Cc1ccc(COc2cccc(C3CCN(Cc4nc5ccc(C(=O)O)cc5n4C[C@@H]4CCO4)CC3)n2)c(F)c1] | 2-[(4-{6-[(4-cyano-2- fluorobenzyl)oxy]pyridin- 2-yl}piperidin-1- yl)methyl]-1-[(2S)-oxetan- 2-ylmethyl]-1H- benzimidazole-6- carboxylic acid |
| 111B | 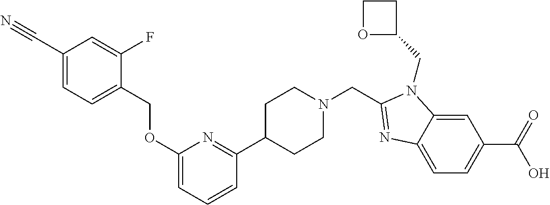 [SMILES: N#Cc1ccc(COc2cccc(C3CCN(Cc4nc5ccc(C(=O)O)cc5n4C[C@H]4CCO4)CC3)n2)c(F)c1] | 2-[(4-{6-[(4-cyano-2- fluorobenzyl)oxy]pyridin- 2-yl}piperidin-1- yl)methyl]-1-[(2R)-oxetan- 2-ylmethyl]-1H- benzimidazole-6- carboxylic acid |
| 112B | 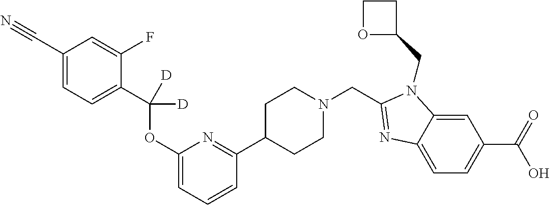 [SMILES: [2H]C([2H])(Oc1cccc(C2CCN(Cc3nc4ccc(C(=O)O)cc4n3C[C@@H]3CCO3)CC2)n1)c1ccc(C#N)cc1F] | 2-{[4-(6-{[(4-cyano-2- fluorophenyl)(methyl- d2)]oxy}pyridin-2-yl) piperidin-1-yl]methyl}- 1-[(2S)-oxetan-2-ylmethyl]- 1H-benzimidazole-6- carboxylic acid |
| 113B | 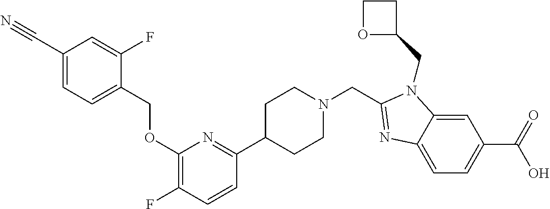 [SMILES: N#Cc1ccc(COc2nc(C3CCN(Cc4nc5ccc(C(=O)O)cc5n4C[C@@H]4CCO4)CC3)ccc2F)c(F)c1] | 2-[(4-{6-[(4-cyano-2- fluorobenzyl)oxy]-5- fluoropyridin-2-yl}piperidin- 1-yl)methyl]-1- [(2S)-oxetan-2-ylmethyl]-1H- benzimidazole-6- carboxylic acid |
| 114B | 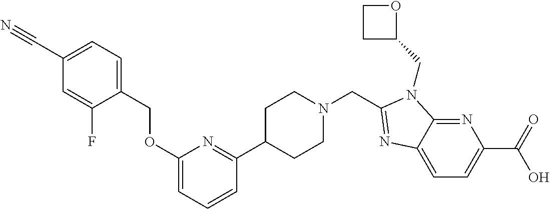 [SMILES: N#Cc1ccc(COc2cccc(C3CCN(Cc4nc5ccc(C(=O)O)nc5n4C[C@@H]4CCO4)CC3)n2)c(F)c1] | 2-[(4-{6-[(4-cyano-2- fluorobenzyl)oxy]pyridin- 2-yl}piperidin-1-yl)methyl]- 3-[(2S)-oxetan-2-ylmethyl]- 3H-imidazo[4,5-b]pyridine- 5-carboxylic acid |
| 115B | 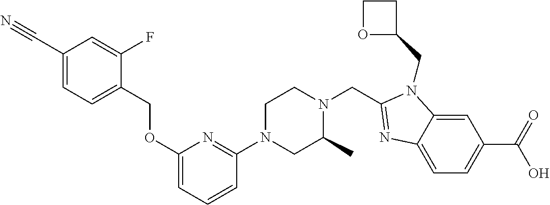 [SMILES: C[C@H]1CN(c2cccc(OCc3ccc(C#N)cc3F)n2)CCN1Cc1nc2ccc(C(=O)O)cc2n1C[C@@H]1CCO1] | 2-{[(2S)-4-{6-[(4-cyano-2- fluorobenzyl)oxy]pyridin- 2-yl}-2-methylpiperazin- 1-yl]methyl}-1-[(2S)- oxetan-2-ylmethyl]-1H- benzimidazole-6- carboxylic acid |
| 116B | 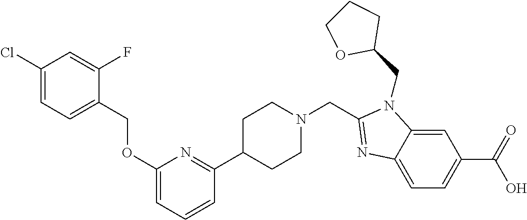 [SMILES: O=C(O)c1ccc2nc(CN3CCC(c4cccc(OCc5ccc(Cl)cc5F)n4)CC3)n(C[C@@H]3CCCO3)c2c1] | 2-[(4-{6-[(4-chloro-2- fluorobenzyl)oxy]pyridin- 2-yl}piperidin-1- yl)methyl]-1-[(2S)- tetrahydrofuran-2- ylmethyl]-1H-benzimidazole- 6-carboxylic acid |
| 117B | 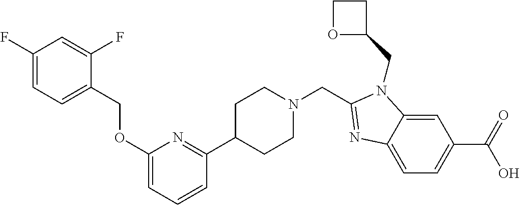 [SMILES: O=C(O)c1ccc2nc(CN3CCC(c4cccc(OCc5ccc(F)cc5F)n4)CC3)n(C[C@@H]3CCO3)c2c1] | 2-[(4-{6-[(2,4-difluorobenzyl) oxy]pyridin-2-yl}piperidin-1- yl)methyl]-1-[(2S)-oxetan-2- ylmethyl]-1H-benzimidazole- 6-carboxylic acid |
| 118B | 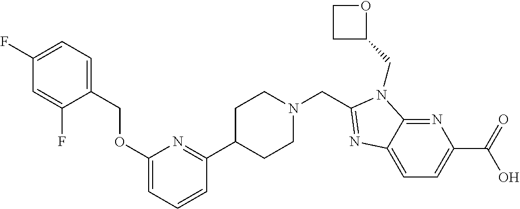 [SMILES: O=C(O)c1ccc2nc(CN3CCC(c4cccc(OCc5ccc(F)cc5F)n4)CC3)n(C[C@@H]3CCO3)c2n1] | 2-[(4-{6-[(2,4-difluorobenzyl) oxy]pyridin-2-yl}piperidin-1- yl)methyl]-3-[(2S)-oxetan-2- ylmethyl]-3H-imidazo[4,5-b] pyridine-5-carboxylic acid |
| 119B | 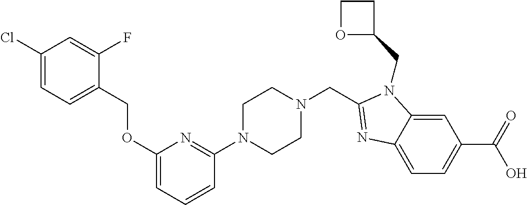 [SMILES: O=C(O)c1ccc2nc(CN3CCN(c4cccc(OCc5ccc(Cl)cc5F)n4)CC3)n(C[C@@H]3CCO3)c2c1] | 2-[(4-{6-[(4-chloro-2- fluorobenzyl)oxy]pyridin- 2-yl}piperazin-1- yl)methyl]-1-[(2S)-oxetan- 2-ylmethyl]-1H- benzimidazole-6- carboxylic acid |
| 120B | 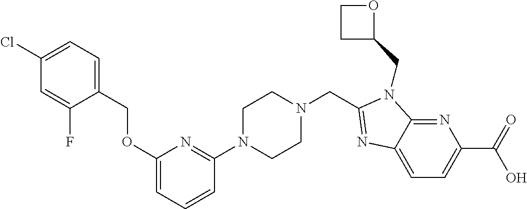 [SMILES: O=C(O)c1ccc2nc(CN3CCN(c4cccc(OCc5ccc(Cl)cc5F)n4)CC3)n(C[C@H]3CCO3)c2n1] | 2-[(4-{6-[(4-chloro-2- fluorobenzyl)oxy] pyridin-2-yl}piperazin-1- yl)methyl]-3-[(2S)-oxetan- 2-ylmethyl]-3H- imidazo[4,5-b]pyridine- 5-carboxylic acid |
| 121B | 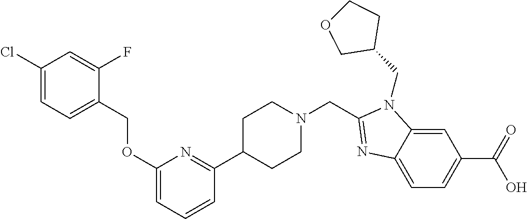 [SMILES: O=C(O)c1ccc2nc(CN3CCC(c4cccc(OCc5ccc(Cl)cc5F)n4)CC3)n(C[C@H]3CCOC3)c2c1] | 2-[(4-{6-[(4-chloro-2- fluorobenzyl)oxy]pyridin- 2-yl}piperidin-1- yl)methyl]-1-[(3R)- tetrahydrofuran-3- ylmethyl]-1H- benzimidazole-6- carboxylic acid |
| 122B | 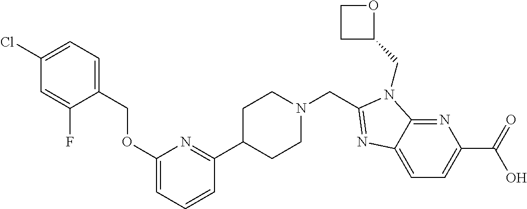 [SMILES: O=C(O)c1ccc2nc(CN3CCC(c4cccc(OCc5ccc(Cl)cc5F)n4)CC3)n(C[C@@H]3CCO3)c2n1] | 2-[(4-{6-[(4-chloro-2- fluorobenzyl)oxy]pyridin- 2-yl}piperidin-1- yl)methyl]-3-[(2S)-oxetan- 2-ylmethyl]-3H- imidazo[4,5-b]pyridine- 5-carboxylic acid |
| 123B | 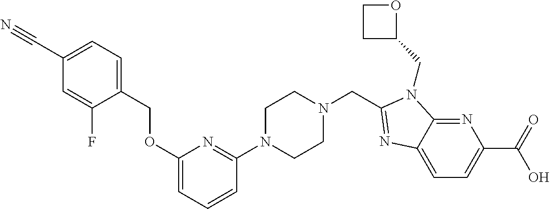 [SMILES: N#Cc1ccc(COc2cccc(N3CCN(Cc4nc5ccc(C(=O)O)nc5n4C[C@@H]4CCO4)CC3)n2)c(F)c1] | 2-[(4-{6-[(4-cyano-2- fluorobenzyl)oxy]pyridin- 2-yl}piperazin-1- yl)methyl]-3-[(2S)-oxetan- 2-ylmethyl]-3H- imidazo[4,5-b]pyridine- 5-carboxylic acid |
| 124B | 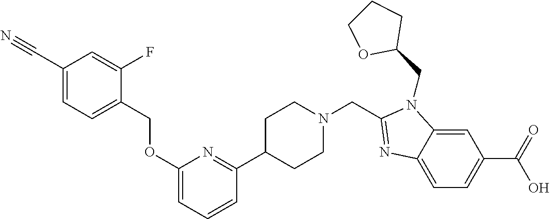 [SMILES: N#Cc1ccc(COc2cccc(C3CCN(Cc4nc5ccc(C(=O)O)cc5n4C[C@@H]4CCCO4)CC3)n2)c(F)c1] | 2-[(4-{6-[(4-cyano-2- fluorobenzyl)oxy]pyridin- 2-yl}piperidin-1- yl)methyl]-1-[(2S)- tetrahydrofuran-2- ylmethyl]-1H-benzimidazole- 6-carboxylic acid |
| 125B | 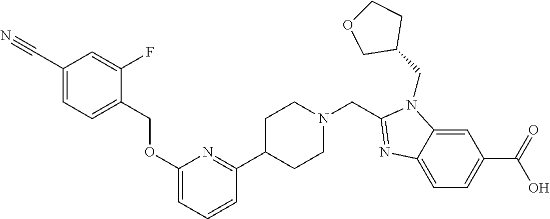 [SMILES: N#Cc1ccc(COc2cccc(C3CCN(Cc4nc5ccc(C(=O)O)cc5n4C[C@H]4CCOC4)CC3)n2)c(F)c1] | 2-[(4-{6-[(4-cyano-2- fluorobenzyl)oxy]pyridin- 2-yl}piperidin-1- yl)methyl]-1-[(3R)- tetrahydrofuran-3- ylmethyl]-1H- benzimidazole-6- carboxylic acid |
| 126B | 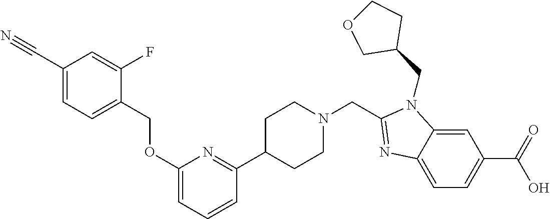 [SMILES: N#Cc1ccc(COc2cccc(C3CCN(Cc4nc5ccc(C(=O)O)cc5n4C[C@@H]4CCOC4)CC3)n2)c(F)c1] | 2-[(4-{6-[(4-cyano-2- fluorobenzyl)oxy]pyridin- 2-yl}piperidin-1- yl)methyl]-1-[(3S)- tetrahydrofuran-3- ylmethyl]-1H- benzimidazole-6- carboxylic acid |
| 127B | 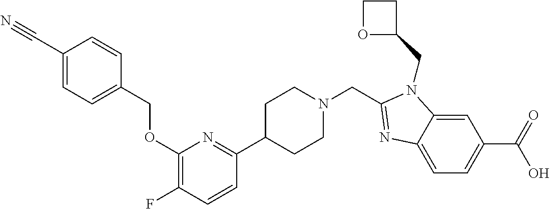 [SMILES: N#Cc1ccc(COc2nc(C3CCN(Cc4nc5ccc(C(=O)O)cc5n4C[C@@H]4CCO4)CC3)ccc2F)cc1] | 2-[(4-{6-[(4-cyanobenzyl) oxy]-5-fluoropyridin-2- yl}piperidin-1-yl)methyl]-1- [(2S)-oxetan-2-ylmethyl]-1H- benzimidazole-6- carboxylic acid |
| 128B | 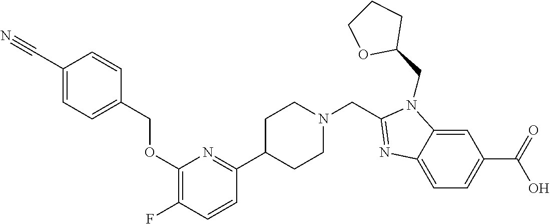 [SMILES: N#Cc1ccc(COc2nc(C3CCN(Cc4nc5ccc(C(=O)O)cc5n4C[C@@H]4CCCO4)CC3)ccc2F)cc1] | 2-[(4-{6-[(4-cyanobenzyl) oxy]-5-fluoropyridin-2- yl}piperidin-1-yl)methyl]-1- [(2S)-tetrahydrofuran-2- ylmethyl]-1H- benzimidazole-6- carboxylic acid |
| 129B | 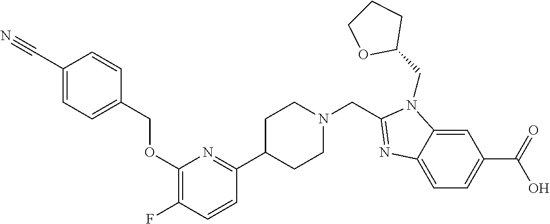 [SMILES: N#Cc1ccc(COc2nc(C3CCN(Cc4nc5ccc(C(=O)O)cc5n4C[C@H]4CCCO4)CC3)ccc2F)cc1] | 2-[(4-{6-[(4-cyanobenzyl) oxy]-5-fluoropyridin-2-yl} piperidin-1-yl)methyl]-1- [(2R)-tetrahydrofuran-2- ylmethyl]-1H- benzimidazole-6- carboxylic acid |
| 130B | 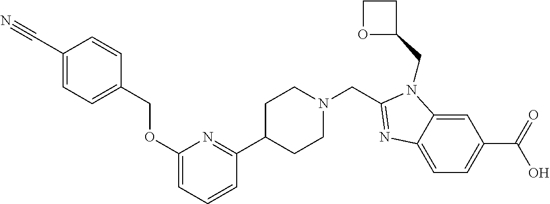 [SMILES: N#Cc1ccc(COc2cccc(C3CCN(Cc4nc5ccc(C(=O)O)cc5n4C[C@@H]4CCO4)CC3)n2)cc1] | 2-[(4-{6-[(4-cyanobenzyl) oxy]pyridin-2-yl}piperidin- 1-yl)methyl]-1-[(2S)-oxetan-2- ylmethyl]-1H-benzimidazole- 6-carboxylic acid |
| 131B | 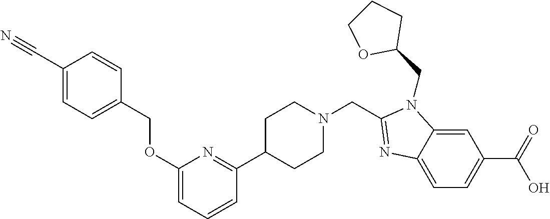 [SMILES: N#Cc1ccc(COc2cccc(C3CCN(Cc4nc5ccc(C(=O)O)cc5n4C[C@@H]4CCCO4)CC3)n2)cc1] | 2-[(4-{6-[(4-cyanobenzyl) oxy]pyridin-2-yl}piperidin- 1-yl)methyl]-1-[(2S)- tetrahydrofuran-2-ylmethyl]- 1H-benzimidazole-6- carboxylic acid |
| 132B | 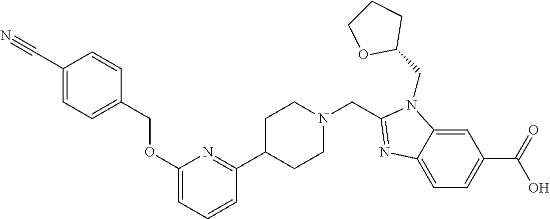 [SMILES: N#Cc1ccc(COc2cccc(C3CCN(Cc4nc5ccc(C(=O)O)cc5n4C[C@H]4CCCO4)CC3)n2)cc1] | 2-[(4-{6-[(4-cyanobenzyl)oxy] pyridin-2-yl}piperidin-1-yl) methyl]-1-[(2R)-tetrahydrofuran- 2-ylmethyl]-1H- benzimidazole-6-carboxylic acid |
| 133B | 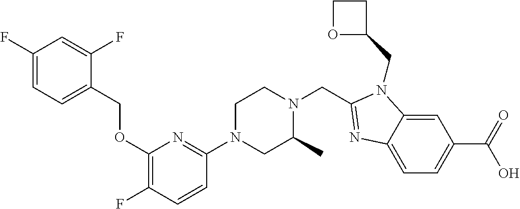 [SMILES: C[C@H]1CN(c2ccc(F)c(OCc3ccc(F)cc3F)n2)CCN1Cc1nc2ccc(C(=O)O)cc2n1C[C@@H]1CCO1] | 2-{[(2S)-4-{6-[(2,4- difluorobenzyl)oxy]-5- fluoropyridin-2-yl}-2- methylpiperazin-1- yl]methyl}-1-[(2S)-oxetan- 2-ylmethyl]-1H- benzimidazole-6- carboxylic acid |
| 134B | 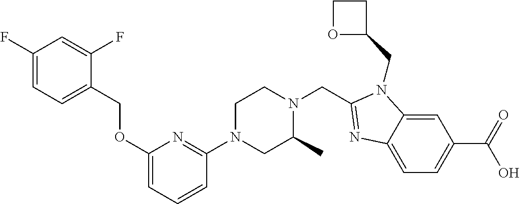 [SMILES: C[C@H]1CN(c2cccc(OCc3ccc(F)cc3F)n2)CCN1Cc1nc2ccc(C(=O)O)cc2n1C[C@@H]1CCO1] | 2-{[(2S)-4-{6-[(2,4- difluorobenzyl)oxy]pyridin- 2-yl}-2-methylpiperazin- 1-yl]methyl}-1-[(2S)- oxetan-2-ylmethyl]-1H- benzimidazole-6- carboxylic acid |
| 135B | 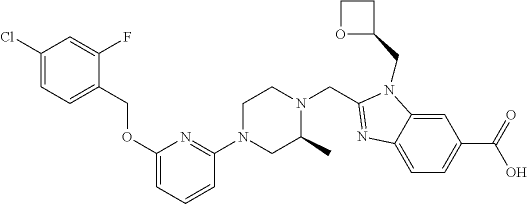 [SMILES: C[C@H]1CN(c2cccc(OCc3ccc(Cl)cc3F)n2)CCN1Cc1nc2ccc(C(=O)O)cc2n1C[C@@H]1CCO1] | 2-{[(2S)-4-{6-[(4-chloro-2- fluorobenzyl)oxy]pyridin- 2-yl}-2-methylpiperazin- 1-yl]methyl}-1-[(2S)- oxetan-2-ylmethyl]-1H- benzimidazole-6- carboxylic acid |
| 136B | 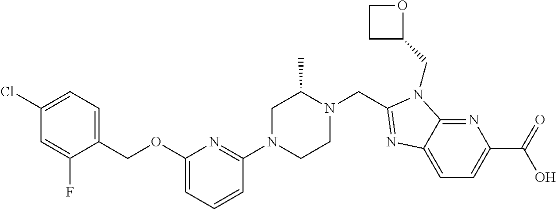 [SMILES: C[C@H]1CN(c2cccc(OCc3ccc(Cl)cc3F)n2)CCN1Cc1nc2ccc(C(=O)O)nc2n1C[C@@H]1CCO1] | 2-{[(2S)-4-{6-[(4-chloro-2- fluorobenzyl)oxy]pyridin- 2-yl}-2-methylpiperazin-1- yl]methyl}-3-[(2S)- oxetan-2-ylmethyl]-3H- imidazo[4,5- b]pyridine-5-carboxylic acid |
| 137B | 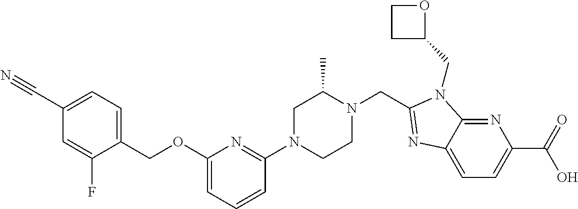 [SMILES: C[C@H]1CN(c2cccc(OCc3ccc(C#N)cc3F)n2)CCN1Cc1nc2ccc(C(=O)O)nc2n1C[C@@H]1CCO1] | 2-{[(2S)-4-{6-[(4-cyano-2- fluorobenzyl)oxy]pyridin-2- yl}-2-methylpiperazin-1- yl]methyl}-3-[(2S)- oxetan-2-ylmethyl]- 3H-imidazo[4,5- b]pyridine-5- carboxylic acid |
| 138B | 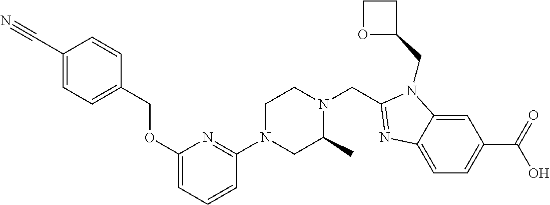 [SMILES: C[C@H]1CN(c2cccc(OCc3ccc(C#N)cc3)n2)CCN1Cc1nc2ccc(C(=O)O)cc2n1C[C@@H]1CCO1] | 2-{[(2S)-4-{6-[(4-cyanobenzyl) oxy]pyridin-2-yl}-2- methylpiperazin-1-yl]methyl}-1- [(2S)-oxetan-2-ylmethyl]-1H- benzimidazole-6-carboxylic acid |
| 139B | 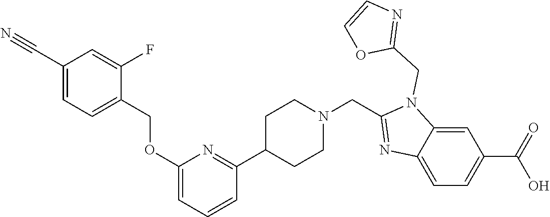 [SMILES: N#Cc1ccc(COc2cccc(C3CCN(Cc4nc5ccc(C(=O)O)cc5n4Cc4ncco4)CC3)n2)c(F)c1] | 2-[(4-{6-[(4-cyano-2- fluorobenzyl)oxy]pyridin- 2-yl}piperidin-1- yl)methyl]-1-(1,3-oxazol- 2-ylmethyl)-1H- benzimidazole-6- carboxylic acid |
| 140B | 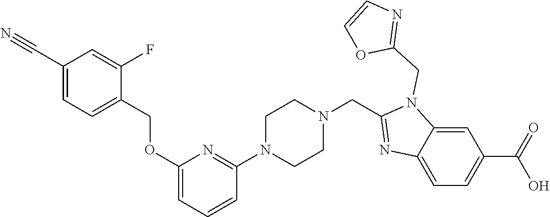 [SMILES: N#Cc1ccc(COc2cccc(N3CCN(Cc4nc5ccc(C(=O)O)cc5n4Cc4ncco4)CC3)n2)c(F)c1] | 2-[(4-{6-[(4-cyano-2- fluorobenzyl)oxy]pyridin- 2-yl}piperazin-1- yl)methyl]-1-(1,3-oxazol- 2-ylmethyl)-1H- benzimidazole-6- carboxylic acid |
| 141B | 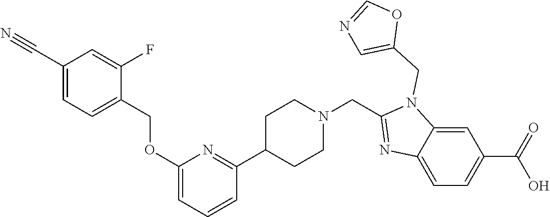 [SMILES: N#Cc1ccc(COc2cccc(C3CCN(Cc4nc5ccc(C(=O)O)cc5n4Cc4cnco4)CC3)n2)c(F)c1] | 2-[(4-{6-[(4-cyano-2- fluorobenzyl)oxy]pyridin- 2-yl}piperidin-1- yl)methyl]-1-(1,3- oxazol-5-ylmethyl)-1H- benzimidazole-6- carboxylic acid |
| 142B | 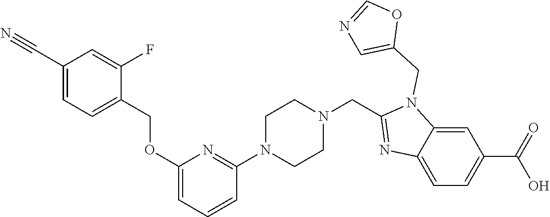 [SMILES: N#Cc1ccc(COc2cccc(N3CCN(Cc4nc5ccc(C(=O)O)cc5n4Cc4cnco4)CC3)n2)c(F)c1] | 2-[(4-{6-[(4-cyano-2- fluorobenzyl)oxy]pyridin- 2-yl}piperazin-1- yl)methyl]-1-(1,3-oxazol- 5-ylmethyl)-1H- benzimidazole-6- carboxylic acid |
| 143B | 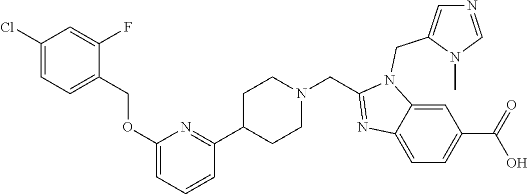 [SMILES: Cn1cncc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[(1-methyl- 1H-imidazol-5-yl)methyl]- 1H-benzimidazole-6- carboxylic acid |
| 144B | 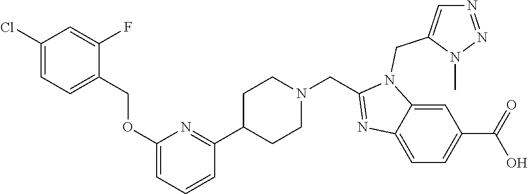 [SMILES: Cn1nncc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[(1-methyl- 1H-1,2,3-triazol-5- yl)methyl]-1H- benzimidazole-6-carboxylic acid |
| 145B | 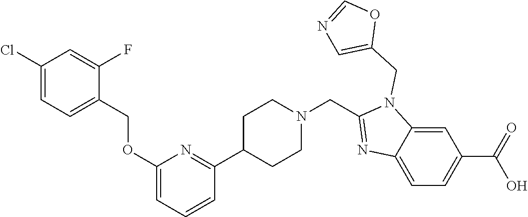 [SMILES: O=C(O)c1ccc2nc(CN3CCC(c4cccc(OCc5ccc(Cl)cc5F)n4)CC3)n(Cc3cnco3)c2c1] | 2-[(4-{6-[(4-chloro-2- fluorobenzyl)oxy]pyridin- 2-yl}piperidin-1- yl)methyl]-1-(1,3-oxazol- 5-ylmethyl)-1H- benzimidazole-6- carboxylic acid |
| 146B | 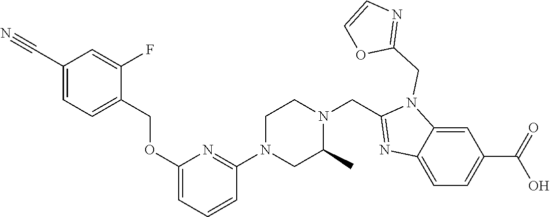 [SMILES: C[C@H]1CN(c2cccc(OCc3ccc(C#N)cc3F)n2)CCN1Cc1nc2ccc(C(=O)O)cc2n1Cc1ncco1] | 2-{[(2S)-4-{6-[(4-cyano-2- fluorobenzyl)oxy]pyridin- 2-yl}-2-methylpiperazin-1- yl]methyl}-1-(1,3-oxazol- 2-ylmethyl)-1H- benzimidazole-6-carboxylic acid |
| 147B | 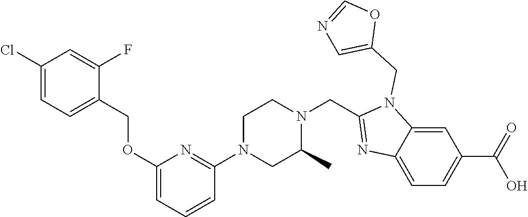 [SMILES: C[C@H]1CN(c2cccc(OCc3ccc(Cl)cc3F)n2)CCN1Cc1nc2ccc(C(=O)O)cc2n1Cc1cnco1] | 2-{[(2S)-4-{6-[(4-chloro-2- fluorobenzyl)oxy]pyridin- 2-yl}-2-methylpiperazin-1- yl]methyl}-1-(1,3-oxazol- 5-ylmethyl)-1H- benzimidazole-6-carboxylic acid |
| 148B | 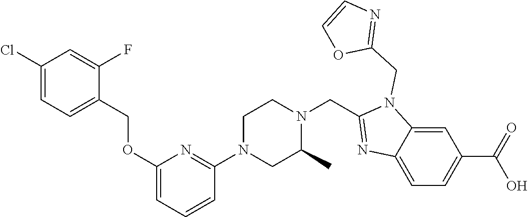 [SMILES: C[C@H]1CN(c2cccc(OCc3ccc(Cl)cc3F)n2)CCN1Cc1nc2ccc(C(=O)O)cc2n1Cc1ncco1] | 2-{[(2S)-4-{6-[(4-chloro-2- fluorobenzyl)oxy]pyridin- 2-yl}-2-methylpiperazin- 1-yl]methyl}-1-(1,3-oxazol- 2-ylmethyl)-1H-benzimidazole- 6-carboxylic acid |
| 149B | 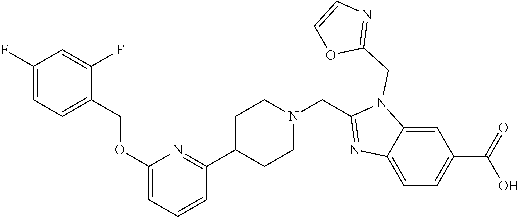 [SMILES: O=C(O)c1ccc2nc(CN3CCC(c4cccc(OCc5ccc(F)cc5F)n4)CC3)n(Cc3ncco3)c2c1] | 2-[(4-{6-[(2,4-difluorobenzyl) oxy]pyridin-2- yl}piperidin-1-yl)methyl]- 1-(1,3-oxazol-2- ylmethyl)-1H-benzimidazole- 6-carboxylic acid |
| 150B | 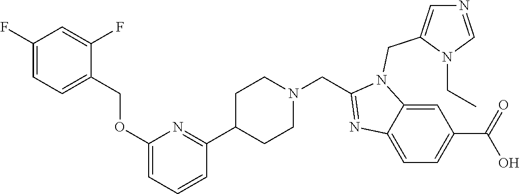 [SMILES: CCn1cncc1Cn1c(CN2CCC(c3cccc(OCc4ccc(F)cc4F)n3)CC2)nc2ccc(C(=O)O)cc21] | 2-[(4-{6-[(2,4-difluorobenzyl) oxy]pyridin-2- yl}piperidin-1-yl)methyl]- 1-[(1-ethyl-1H- imidazol-5-yl)methyl]-1H- benzimidazole-6- carboxylic acid |
| 151B | 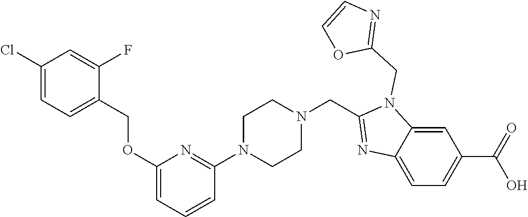 [SMILES: O=C(O)c1ccc2nc(CN3CCN(c4cccc(OCc5ccc(Cl)cc5F)n4)CC3)n(Cc3ncco3)c2c1] | 2-[(4-{6-[(4-chloro-2- fluorobenzyl)oxy]pyridin- 2-yl}piperazin-1- yl)methyl]-1-(1,3-oxazol- 2-ylmethyl)-1H- benzimidazole-6- carboxylic acid |
| 152B | 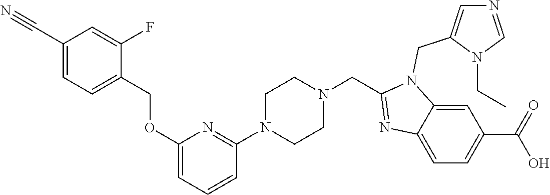 [SMILES: CCn1cncc1Cn1c(CN2CCN(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperazin-1- yl)methyl]-1-[(1-ethyl- 1H-imidazol-5- yl)methyl]-1H- benzimidazole-6-carboxylic acid |
| 153B | 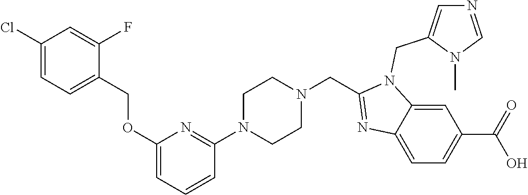 [SMILES: Cn1cncc1Cn1c(CN2CCN(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperazin-1-yl)methyl]- 1-[(1-methyl-1H-imidazol-5- yl)methyl]-1H- benzimidazole-6-carboxylic acid |
| 154B | 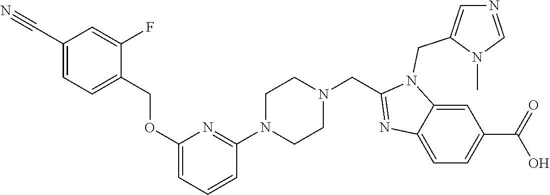 [SMILES: Cn1cncc1Cn1c(CN2CCN(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperazin-1- yl)methyl]-1-[(1-methyl- 1H-imidazol-5- yl)methyl]-1H- benzimidazole-6- carboxylic acid |
| 155B | 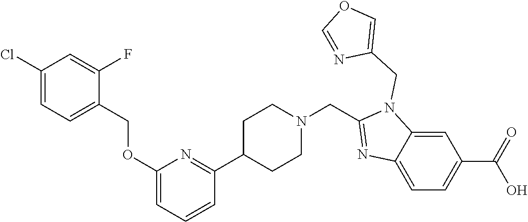 [SMILES: O=C(O)c1ccc2nc(CN3CCC(c4cccc(OCc5ccc(Cl)cc5F)n4)CC3)n(Cc3cocn3)c2c1] | 2-[(4-{6-[(4-chloro-2- fluorobenzyl)oxy]pyridin- 2-yl}piperidin-1- yl)methyl]-1-(1,3-oxazol- 4-ylmethyl)-1H- benzimidazole-6- carboxylic acid |
| 156B | 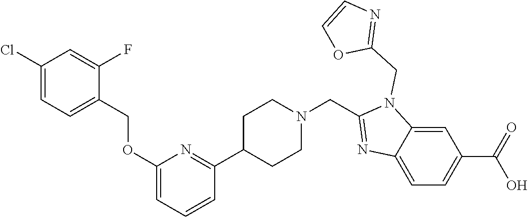 [SMILES: O=C(O)c1ccc2nc(CN3CCC(c4cccc(OCc5ccc(Cl)cc5F)n4)CC3)n(Cc3ncco3)c2c1] | 2-[(4-{6-[(4-chloro-2- fluorobenzyl)oxy]pyridin- 2-yl}piperidin-1- yl)methyl]-1-(1,3-oxazol- 2-ylmethyl)-1H- benzimidazole-6- carboxylic acid |
| 157B | 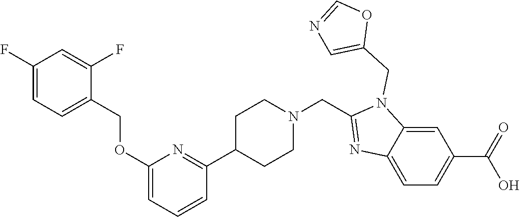 [SMILES: O=C(O)c1ccc2nc(CN3CCC(c4cccc(OCc5ccc(F)cc5F)n4)CC3)n(Cc3cnco3)c2c1] | 2-[(4-{6-[(2,4-difluorobenzyl) oxy]pyridin-2- yl}piperidin-1-yl)methyl]- 1-(1,3-oxazol-5- ylmethyl)-1H-benzimidazole- 6-carboxylic acid |
| 158B | 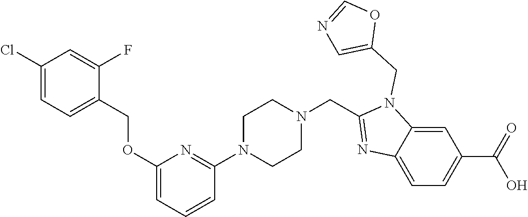 [SMILES: O=C(O)c1ccc2nc(CN3CCN(c4cccc(OCc5ccc(Cl)cc5F)n4)CC3)n(Cc3cnco3)c2c1] | 2-[(4-{6-[(4-chloro-2- fluorobenzyl)oxy]pyridin- 2-yl}piperazin-1- yl)methyl]-1-(1,3-oxazol- 5-ylmethyl)-1H- benzimidazole-6- carboxylic acid |
| 159B | 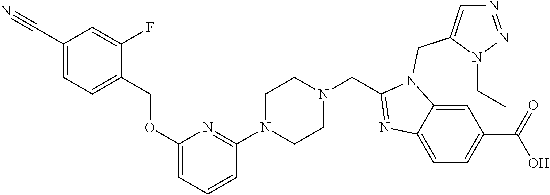 [SMILES: CCn1nncc1Cn1c(CN2CCN(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperazin-1- yl)methyl]-1-[(1-ethyl-1H- 1,2,3-triazol-5- yl)methyl]-1H-benzimidazole- 6-carboxylic acid |
| 160B | 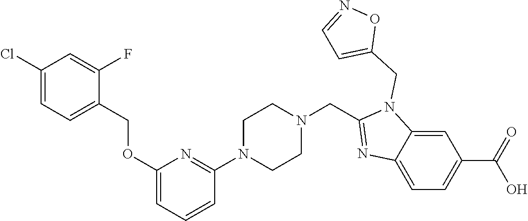 [SMILES: O=C(O)c1ccc2nc(CN3CCN(c4cccc(OCc5ccc(Cl)cc5F)n4)CC3)n(Cc3ccno3)c2c1] | 2-[(4-{6-[(4-chloro-2- fluorobenzyl)oxy] pyridin-2-yl}piperidin-1- yl)methyl]-1-(1,2-oxazol- 5-ylmethyl)-1H- benzimidazole-6- carboxylic acid |
| 161B | 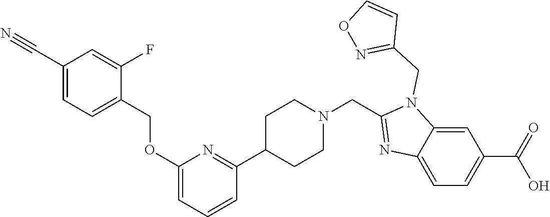 [SMILES: N#Cc1ccc(COc2cccc(C3CCN(Cc4nc5ccc(C(=O)O)cc5n4Cc4ccon4)CC3)n2)c(F)c1] | 2-[(4-{6-[(4-cyano-2- fluorobenzyl)oxy]pyridin- 2-yl}piperidin-1- yl)methyl]-1-(1,2-oxazol- 3-ylmethyl)-1H- benzimidazole-6- carboxylic acid |
| 162B | 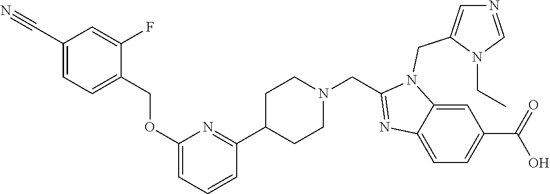 [SMILES: CCn1cncc1Cn1c(CN2CCC(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperidin-1- yl)methyl]-1-[(1-ethyl- 1H-imidazol-5- yl)methyl]-1H- benzimidazole-6-carboxylic acid |
| 163B | 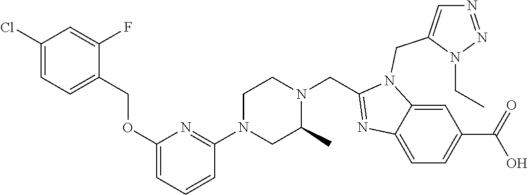 [SMILES: CCn1nncc1Cn1c(CN2CCN(c3cccc(OCc4ccc(Cl)cc4F)n3)C[C@@H]2C)nc2ccc(C(=O)O)cc21] | 2-{[(2S)-4-{6-[(4-chloro-2- fluorobenzyl)oxy]pyridin- 2-yl}-2-methylpiperazin-1- yl]methyl}-1-[(1-ethyl- 1H-imidazol-5-yl)methyl]- 1H-benzimidazole-6- carboxylic acid |
| 164B | 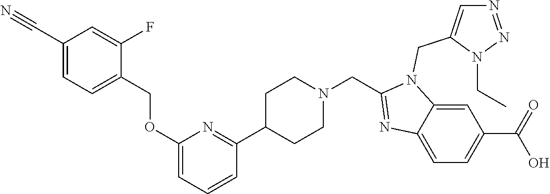 [SMILES: CCn1nncc1Cn1c(CN2CCC(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperidin-1- yl)methyl]-1-[(1-ethyl- 1H-1,2,3-triazol-5- yl)methyl]-1H- benzimidazole-6-carboxylic acid |
| 165B | 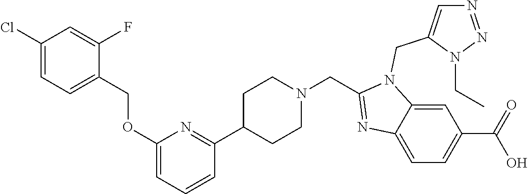 [SMILES: CCn1nncc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[(1-ethyl- 1H-imidazol-5- yl)methyl]-1H- benzimidazole-6-carboxylic acid |
| 166B | 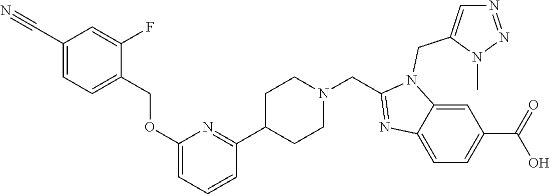 [SMILES: Cn1nncc1Cn1c(CN2CCC(c3cccc(OCc4ccc(C#N)cc4F)n3)CC2)nc2ccc(C(=O)O)cc21] | 2-[(4-{6-[(4-cyano-2- fluorobenzyl)oxy]pyridin- 2-yl}piperidin-1- yl)methyl]-1-[(1-methyl- 1H-imidazol-5- yl)methyl]-1H- benzimidazole-6-carboxylic acid |
| 167B | 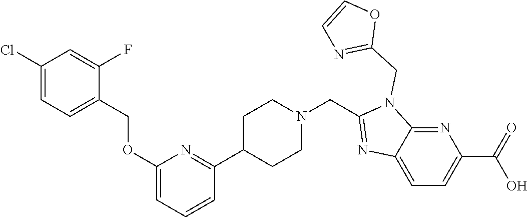 [SMILES: O=C(O)c1ccc2nc(CN3CCC(c4cccc(OCc5ccc(Cl)cc5F)n4)CC3)n(Cc3ncco3)c2n1] | 2-[(4-{6-[(4-chloro-2- fluorobenzyl)oxy]pyridin- 2-yl}piperidin-1- yl)methyl]-3-(1,3-oxazol- 2-ylmethyl)-3H- imidazo[4,5-b]pyridine- 5-carboxylic acid |
| 168B | 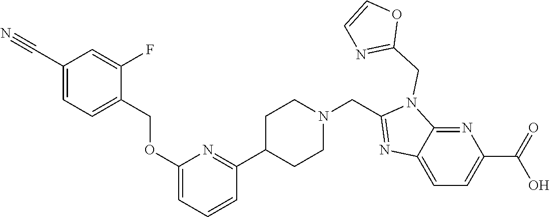 [SMILES: N#Cc1ccc(COc2cccc(C3CCN(Cc4nc5ccc(C(=O)O)nc5n4Cc4ncco4)CC3)n2)c(F)c1] | 2-[(4-{6-[(4-cyano-2- fluorobenzyl)oxy]pyridin- 2-yl}piperidin-1- yl)methyl]-3-(1,3-oxazol- 2-ylmethyl)-3H- imidazo[4,5-b]pyridine-5- carboxylic acid |
| 169B | 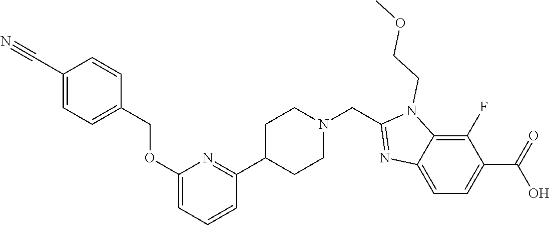 [SMILES: COCCn1c(CN2CCC(c3cccc(OCc4ccc(C#N)cc4)n3)CC2)nc2ccc(C(=O)O)c(F)c21] | 2-[(4-{6-[(4-cyanobenzyl) oxy]pyridin-2- yl}piperidin-1-yl) methyl]-7-fluoro-1-(2- methoxyethyl)-1H- benzimidazole-6- carboxylic acid |
| 170B | 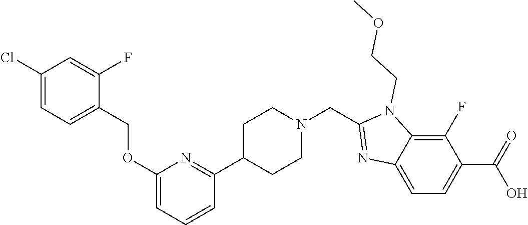 [SMILES: COCCn1c(CN2CCC(c3cccc(OCc4ccc(Cl)cc4F)n3)CC2)nc2ccc(C(=O)O)c(F)c21] | 2-[(4-{6-[(4-chloro-2- fluorobenzyl)oxy]pyridin- 2-yl}piperidin-1- yl)methyl]-7-fluoro-1- (2-methoxyethyl)-1H- benzimidazole-6- carboxylic acid |
| 171B | 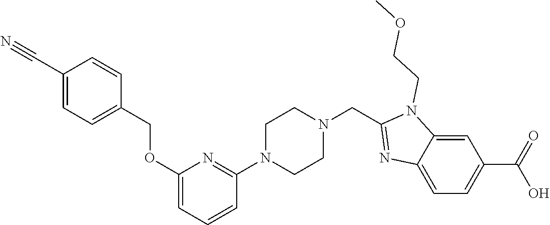 [SMILES: COCCn1c(CN2CCN(c3cccc(OCc4ccc(C#N)cc4)n3)CC2)nc2ccc(C(=O)O)cc21] | 2-[(4-{6-[(4-cyanobenzyl) oxy]pyridin-2- yl}piperazin-1- yl)methyl]-1-(2- methoxyethyl)-1H- benzimidazole-6- carboxylic acid |
| 172B | 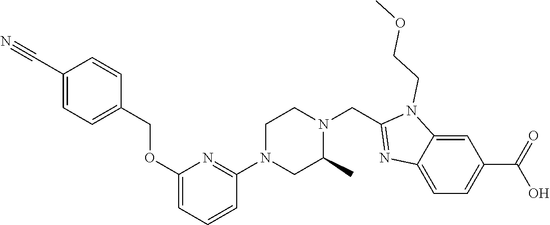 [SMILES: COCCn1c(CN2CCN(c3cccc(OCc4ccc(C#N)cc4)n3)C[C@@H]2C)nc2ccc(C(=O)O)cc21] | 2-{[(2S)-4-{6-[(4- cyanobenzyl)oxy]pyridin- 2-yl}-2-methylpiperazin- 1-yl]methyl}-1-(2- methoxyethyl)-1H- benzimidazole-6- carboxylic acid |
| 173B | 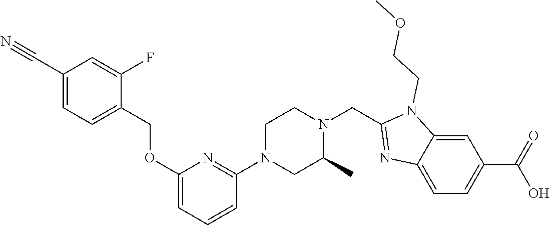 [SMILES: COCCn1c(CN2CCN(c3cccc(OCc4ccc(C#N)cc4F)n3)C[C@@H]2C)nc2ccc(C(=O)O)cc21] | 2-{[(2S)-4-{6-[(4-cyano-2- fluorobenzyl)oxy]pyridin- 2-yl}-2-methylpiperazin- 1-yl]methyl}-1-(2- methoxyethyl)-1H- benzimidazole-6- carboxylic acid |
| 174B | 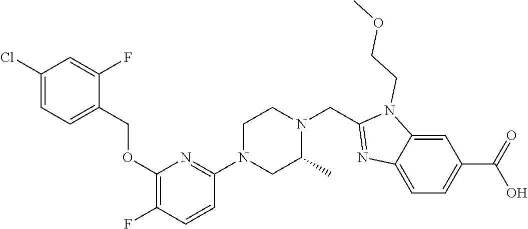 [SMILES: COCCn1c(CN2CCN(c3ccc(F)c(OCc4ccc(Cl)cc4F)n3)C[C@H]2C)nc2ccc(C(=O)O)cc21] | 2-{[(2S)-4-{6-[(4-chloro-2- fluorobenzyl)oxy]-5- fluoropyridin-2-yl}-2- methylpiperazin-1- yl]methyl}-1-(2- methoxyethyl)-1H- benzimidazole-6- carboxylic acid |
| 175B | 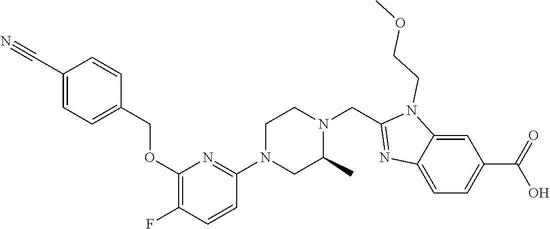 [SMILES: COCCn1c(CN2CCN(c3ccc(F)c(OCc4ccc(C#N)cc4)n3)C[C@@H]2C)nc2ccc(C(=O)O)cc21] | 2-{[(2S)-4-{6-[(4- cyanobenzyl)oxy]-5- fluoropyridin-2-yl}-2- methylpiperazin-1- yl]methyl}-1-(2- methoxyethyl)-1H- benzimidazole-6- carboxylic acid |
| 176B | 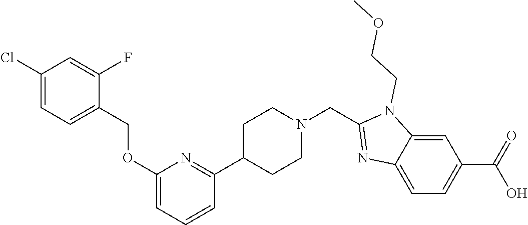 [SMILES: COCCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-(2- methoxyethyl)-1H- benzimidazole-6- carboxylic acid |
| 177B | 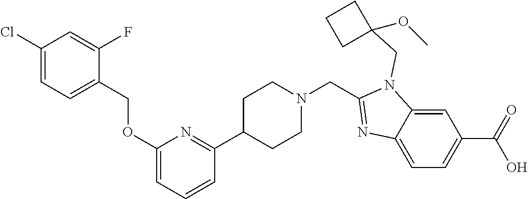 [SMILES: COC1(Cn2c(CN3CCC(c4cccc(OCc5ccc(Cl)cc5F)n4)CC3)nc3ccc(C(=O)O)cc32)CCC1] | 2-[(4-{6-[(4-chloro-2- fluorobenzyl)oxy]pyridin- 2-yl}piperidin-1- yl)methyl]-1-[(1- methoxycyclobutyl)methyl]- 1H-benzimidazole-6- carboxylic acid |
| 178B | 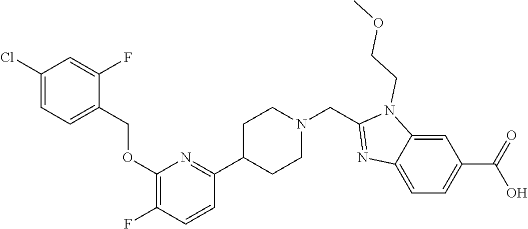 [SMILES: COCCn1c(CN2CCC(c3ccc(F)c(OCc4ccc(Cl)cc4F)n3)CC2)nc2ccc(C(=O)O)cc21] | 2-[(4-{6-[(4-chloro-2- fluorobenzyl)oxy]-5- fluoropyridin-2-yl}piperidin- 1-yl)methyl]-1- (2-methoxyethyl)-1H- benzimidazole-6- carboxylic acid |
| 179B | 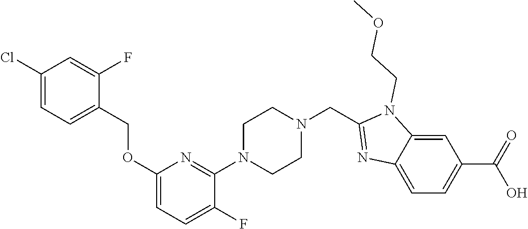 [SMILES: COCCn1c(CN2CCN(c3nc(OCc4ccc(Cl)cc4F)ccc3F)CC2)nc2ccc(C(=O)O)cc21] | 2-[(4-{6-[(4-chloro-2- fluorobenzyl)oxy]-3- fluoropyridin-2-yl}piperazin- 1-yl)methyl]-1- (2-methoxyethyl)-1H- benzimidazole-6- carboxylic acid |
| 180B | 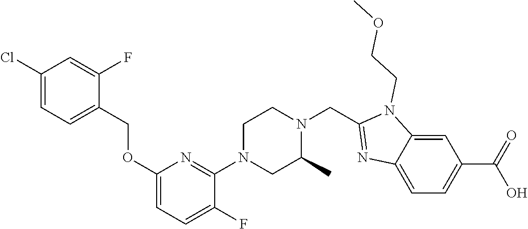 [SMILES: COCCn1c(CN2CCN(c3nc(OCc4ccc(Cl)cc4F)ccc3F)C[C@@H]2C)nc2ccc(C(=O)O)cc21] | 2-{[(2S)-4-{6-[(4-chloro-2- fluorobenzyl)oxy]-3- fluoropyridin-2-yl}-2- methylpiperazin-1-yl] methyl}-1-(2-methoxyethyl)- 1H-benzimidazole-6- carboxylic acid |
| 181B | 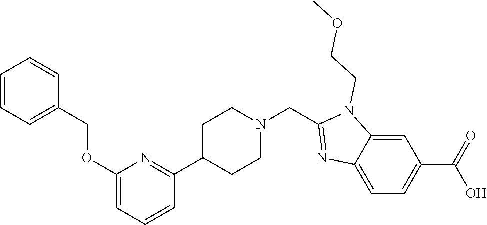 [SMILES: COCCn1c(CN2CCC(c3cccc(OCc4ccccc4)n3)CC2)nc2ccc(C(=O)O)cc21] | 2-({4-[6-(benzyloxy) pyridin-2-yl]piperidin-1- yl}methyl)-1-(2- methoxyethyl)-1H- benzimidazole-6- carboxylic acid |
| 182B | 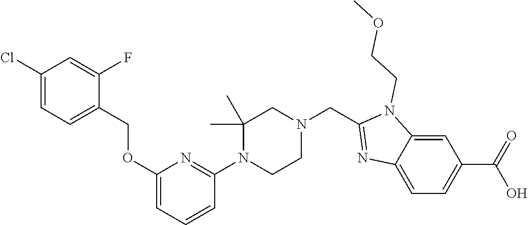 [SMILES: COCCn1c(CN2CCN(c3cccc(OCc4ccc(Cl)cc4F)n3)C(C)(C)C2)nc2ccc(C(=O)O)cc21] | 2-[(4-{6-[(4-chloro-2- fluorobenzyl)oxy]pyridin- 2-yl}-3,3-dimethylpiperazin- 1-yl)methyl]-1-(2- methoxyethyl)-1H- benzimidazole-6- carboxylic acid |
| 183B | 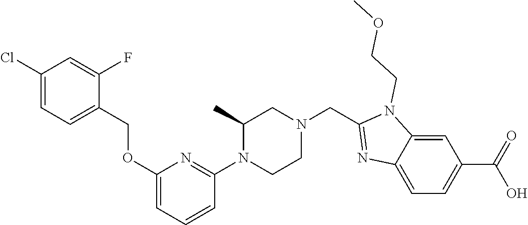 [SMILES: COCCn1c(CN2CCN(c3cccc(OCc4ccc(Cl)cc4F)n3)[C@@H](C)C2)nc2ccc(C(=O)O)cc21] | 2-{[(3S)-4-{6-[(4-chloro-2- fluorobenzyl)oxy]pyridin- 2-yl}-3-methylpiperazin- 1-yl]methyl}-1-(2- methoxyethyl)-1H- benzimidazole-6- carboxylic acid |
| 184B | 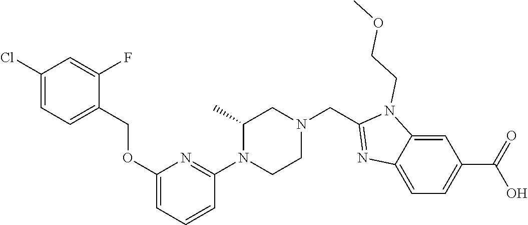 [SMILES: COCCn1c(CN2CCN(c3cccc(OCc4ccc(Cl)cc4F)n3)[C@H](C)C2)nc2ccc(C(=O)O)cc21] | 2-{[(3R)-4-{6-[(4-chloro-2- fluorobenzyl)oxy]pyridin- 2-yl}-3-methylpiperazin-1- yl]methyl}-1-(2- methoxyethyl)-1H- benzimidazole-6- carboxylic acid |
| 185B | 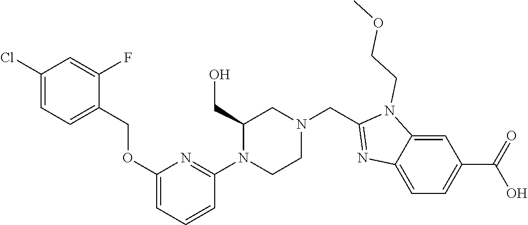 [SMILES: COCCn1c(CN2CCN(c3cccc(OCc4ccc(Cl)cc4F)n3)[C@@H](CO)C2)nc2ccc(C(=O)O)cc21] | 2-{[(3R)-4-{6-[(4-chloro-2- fluorobenzyl)oxy]pyridin-2- yl}-3-(hydroxymethyl) piperazin-1-yl]methyl}-1- (2-methoxyethyl)-1H- benzimidazole-6- carboxylic acid |
| 186B | 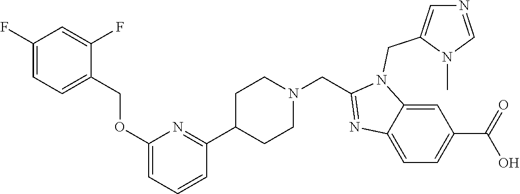 [SMILES: Cn1cncc1Cn1c(CN2CCC(c3cccc(OCc4ccc(F)cc4F)n3)CC2)nc2ccc(C(=O)O)cc21] | 2-[(4-{6-[(2,4-difluorobenzyl) oxy]pyridin-2- yl}piperidin-1-yl)methyl]- 1-[(1-methyl-1H- imidazol-5-yl)methyl]- 1H-benzimidazole-6- carboxylic acid |
| 187B | 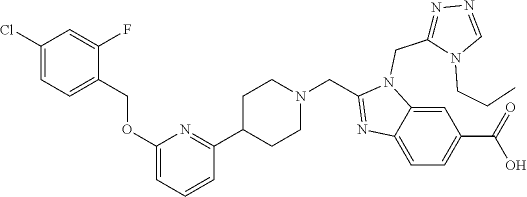 [SMILES: CCCn1cnnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy] pyridin-2-yl}piperidin-1- yl)methyl]-1-[(4-propyl- 4H-1,2,4-triazol-3- yl)methyl]-1H- benzimidazole-6- carboxylic acid |
| 188B | 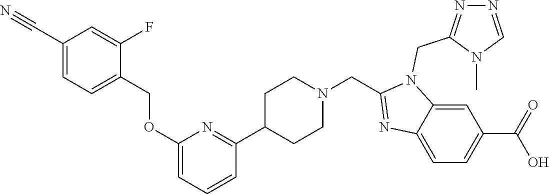 [SMILES: Cn1cnnc1Cn1c(CN2CCC(c3cccc(OCc4ccc(C#N)cc4F)n3)CC2)nc2ccc(C(=O)O)cc21] | 2-[(4-{6-[(4-cyano-2- fluorobenzyl)oxy] pyridin-2-yl}piperidin-1- yl)methyl]-1-[(4-methyl- 4H-1,2,4-triazol-3- yl)methyl]-1H- benzimidazole-6- carboxylic acid |
| 189B | 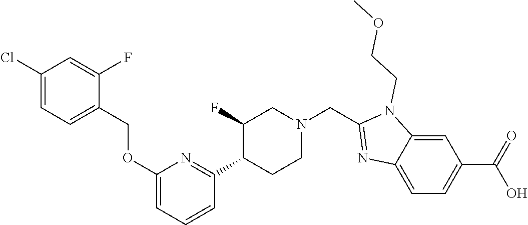 [SMILES: COCCn1c(CN2CC[C@H](c3cccc(OCc4ccc(Cl)cc4F)n3)[C@@H](F)C2)nc2ccc(C(=O)O)cc21] | rac 2-{[(3S,4S)-4-{6- [(4-chloro-2- fluorobenzyl)oxy]pyridin- 2-yl}-3-fluoropiperidin- 1-yl]methyl}-1-(2- methoxyethyl)-1H- benzimidazole-6- carboxylic acid |
| 190B | 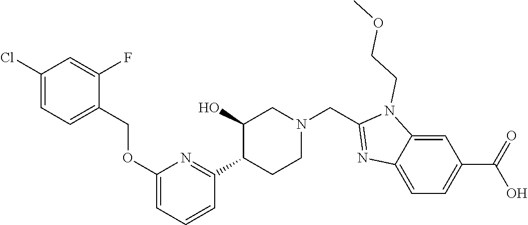 [SMILES: COCCn1c(CN2CC[C@H](c3cccc(OCc4ccc(Cl)cc4F)n3)[C@@H](O)C2)nc2ccc(C(=O)O)cc21] | rac-2-{[(3S,4S)-4-{6- [(4-chloro-2- fluorobenzyl)oxy]pyridin- 2-yl}-3-hydroxypiperidin- 1-yl]methyl}-1-(2- methoxyethyl)-1H- benzimidazole-6- carboxylic acid |
| 191B | 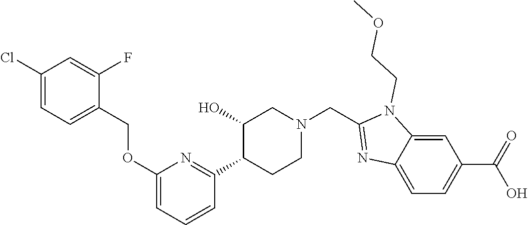 [SMILES: COCCn1c(CN2CC[C@H](c3cccc(OCc4ccc(Cl)cc4F)n3)[C@H](O)C2)nc2ccc(C(=O)O)cc21] | rac-2-{[(3R,4S)-4-{6- [(4-chloro-2- fluorobenzyl)oxy]pyridin- 2-yl}-3- hydroxypiperidin-1-yl] methyl}- 1-(2- methoxyethyl)-1H- benzimidazole-6- carboxylic acid |
| 192B | 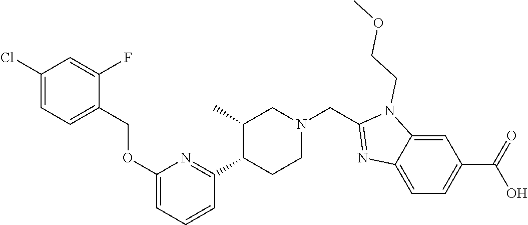 [SMILES: COCCn1c(CN2CC[C@H](c3cccc(OCc4ccc(Cl)cc4F)n3)[C@H](C)C2)nc2ccc(C(=O)O)cc21] | rac-2-{[(3R,4R)-4-{6- [(4-chloro-2- fluorobenzyl)oxy]pyridin- 2-yl}-3-methylpiperidin- 1-yl]methyl}-1-(2- methoxyethyl)-1H- benzimidazole-6- carboxylic acid |
| 193B | 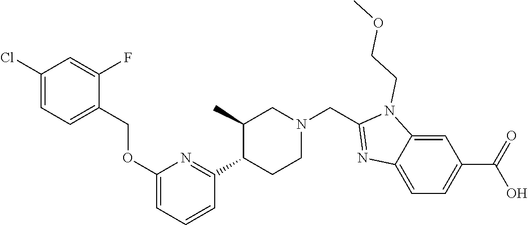 [SMILES: COCCn1c(CN2CC[C@H](c3cccc(OCc4ccc(Cl)cc4F)n3)[C@@H](C)C2)nc2ccc(C(=O)O)cc21] | rac-2-{[(3S,4R)-4-{6- [(4-chloro-2-fluorobenzyl) oxy]pyridin-2-yl}-3- methylpiperidin-1-yl] methyl}-1-(2- methoxyethyl)-1H- benzimidazole-6- carboxylic acid |
| 194B | 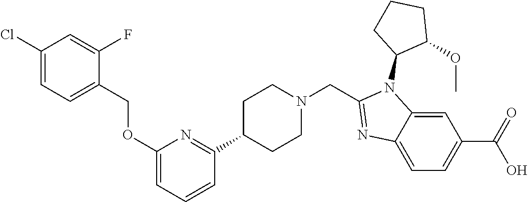 | 2-[(4-{6-[(4-chloro-2- fluorobenzyl)oxy]pyridin- 2-yl}piperidin-1- yl)methyl]-1-[(1R,2R)-2- methoxycyclopentyl]-1H- benzimidazole-6- carboxylic acid |
| 195B | 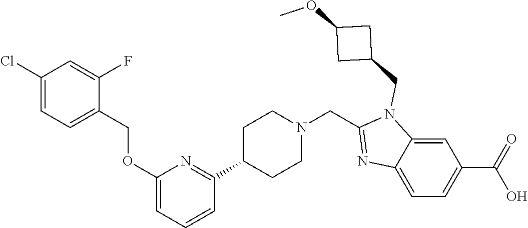 | 2-[(4-{6-[(4-chloro-2- fluorobenzyl)oxy]pyridin- 2-yl}piperidin-1- yl)methyl]-1-[(cis-3- methoxycyclobutyl) methyl]-1H- benzimidazole-6- carboxylic acid |
| 196B | 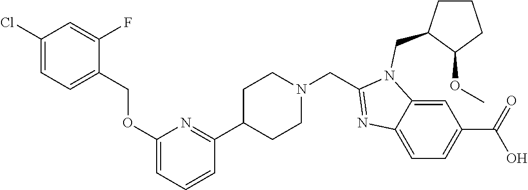 [SMILES: CO[C@@H]1CCC[C@@H]1Cn1c(CN2CCC(c3cccc(OCc4ccc(Cl)cc4F)n3)CC2)nc2ccc(C(=O)O)cc21] | rac-2-[(4-{6-[(4-chloro-2- fluorobenzyl)oxy] pyridin-2-yl}piperidin-1- yl)methyl]-1-{[(1S,2S)-2- methoxycyclopentyl]methyl}- 1H-benzimidazole-6- carboxylic acid |
| 197B | 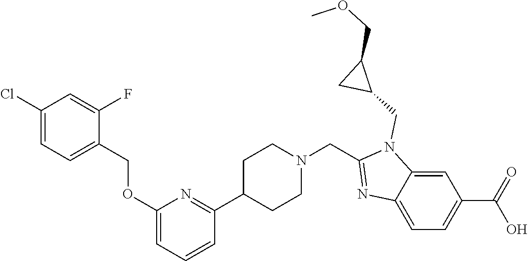 [SMILES: COC[C@@H]1C[C@H]1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl} piperidin-1- yl)methyl]-1-{[(1R,2R)-2- (methoxymethyl) cyclopropyl]methyl}-1H- benzimidazole-6- carboxylic acid |
| 198B | 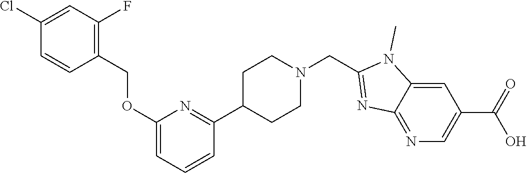 [SMILES: Cn1c(CN2CCC(c3cccc(OCc4ccc(Cl)cc4F)n3)CC2)nc2ncc(C(=O)O)cc21] | 2-((4-(6-((4-Chloro-2- fluorobenzyl)oxy)pyridin- 2-yl)piperidin-1- yl)methyl)-1-methyl- 1H-benzo[d]imidazole- 6-carboxylic acid hydrochloride |
| 199B | 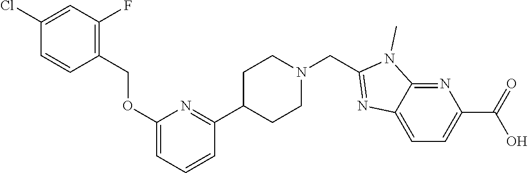 [SMILES: Cn1c(CN2CCC(c3cccc(OCc4ccc(Cl)cc4F)n3)CC2)nc2ccc(C(=O)O)nc21] | 2-((4-(6-((4-Chloro-2- fluorobenzyl)oxy)pyridin- 2-yl)piperidin-1- yl)methyl)-3-methyl- 3H-imidazo[4,5- b]pyridine-5-carboxylic acid hydrochloride |
| 200B | 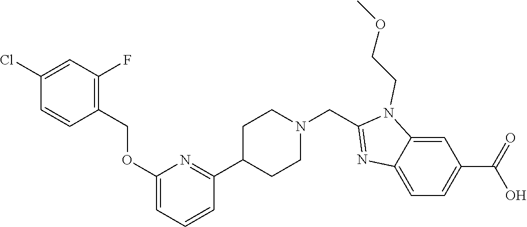 [SMILES: COCCn1c(CN2CCC(c3cccc(OCc4ccc(Cl)cc4F)n3)CC2)nc2ccc(C(=O)O)cc21] | 2-((4-(6-((4-Chloro-2- fluorobenzyl)oxy)pyridin- 2-yl)piperidin-1- yl)methyl)-1-(2- methoxyethyl)-1H- imidazo[4,5-b]pyridine- 6-carboxylic acid hydrochloride |
| 201B | 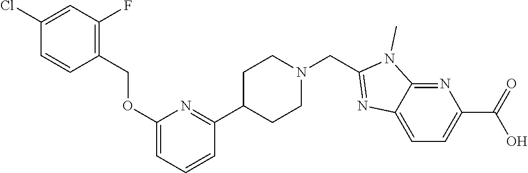 [SMILES: Cn1c(CN2CCC(c3cccc(OCc4ccc(Cl)cc4F)n3)CC2)nc2ccc(C(=O)O)nc21] | 2-((4-(6-((4-Chloro-2- fluorobenzyl)oxy)pyridin- 2-yl)piperidin-1- yl)methyl)-1-methyl- 1H-imidazo[4,5- c]pyridine-6- carboxylic acid |
| 202B | 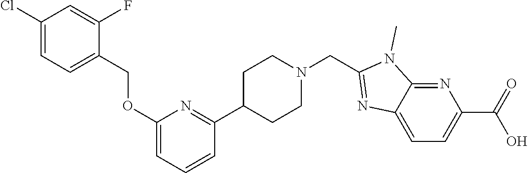 [SMILES: Cn1c(CN2CCC(c3cccc(OCc4ccc(Cl)cc4F)n3)CC2)nc2ccc(C(=O)O)nc21] | 2-[(4-{6-[(4-chloro-2- fluorobenzyl)oxy]pyridin- 2-yl}piperidin-1- yl)methyl]-1-methyl- 1H-imidazo[4,5- b]pyridine-6- carboxylic acid |
| 203B | 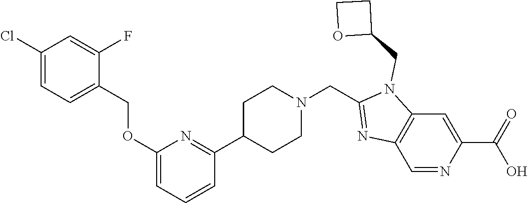 [SMILES: O=C(O)c1cc2c(cn1)nc(CN1CCC(c3cccc(OCc4ccc(Cl)cc4F)n3)CC1)n2C[C@@H]1CCO1] | 2-[(4-{6-[(4-chloro-2- fluorobenzyl)oxy]pyridin- 2-yl}piperidin-1- yl)methyl]-1-[(2S)-oxetan- 2-ylmethyl]-1H- imidazo[4,5-c]pyridine- 6-carboxylic acid |
| 204B | 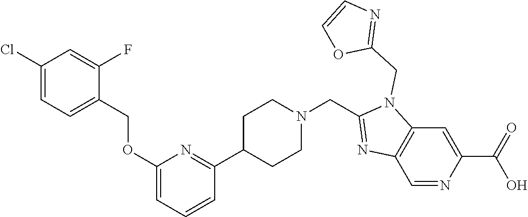 [SMILES: O=C(O)c1cc2c(cn1)nc(CN1CCC(c3cccc(OCc4ccc(Cl)cc4F)n3)CC1)n2Cc1ncco1] | 2-[(4-{6-[(4-chloro-2- fluorobenzyl)oxy]pyridin- 2-yl}piperidin-1- yl)methyl]-1-(1,3-oxazol- 2-ylmethyl)-1H- imidazo[4,5-c]pyridine- 6-carboxylic acid |
| 205B | 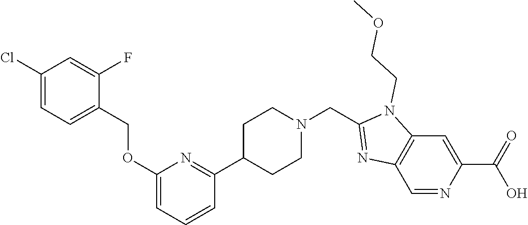 [SMILES: COCCn1c(CN2CCC(c3cccc(OCc4ccc(Cl)cc4F)n3)CC2)nc2cnc(C(=O)O)cc21] | 2-[(4-{6-[(4-chloro-2- fluorobenzyl)oxy]pyridin- 2-yl}piperidin-1- yl)methyl]-1-(2- methoxyethyl)-1H- imidazo[4,5-c]pyridine- 6-carboxylic acid |
| 206B | 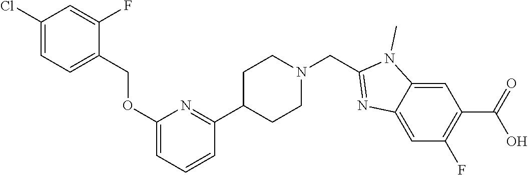 [SMILES: Cn1c(CN2CCC(c3cccc(OCc4ccc(Cl)cc4F)n3)CC2)nc2cc(F)c(C(=O)O)cc21] | 2-[(4-{6-[(4-chloro-2- fluorobenzyl)oxy]pyridin- 2-yl} piperidin-1- yl)methyl]-5-fluoro- 1-methyl-1H- benzimidazole-6- carboxylic acid |
| 207B | 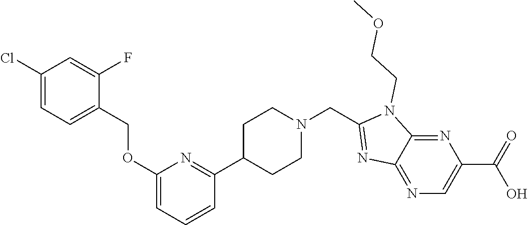 [SMILES: COCCn1c(CN2CCC(c3cccc(OCc4ccc(Cl)cc4F)n3)CC2)nc2ncc(C(=O)O)nc21] | 2-[(4-{6-[(4-chloro-2- fluorobenzyl)oxy]pyridin- 2-yl}piperidin-1- yl)methyl]-1-(2- methoxyethyl)-1H- imidazo[4,5-b]pyrazine- 6-carboxylic acid |
| 208B | 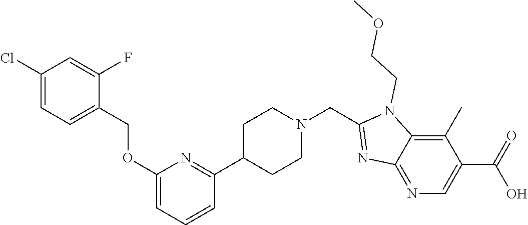 [SMILES: COCCn1c(CN2CCC(c3cccc(OCc4ccc(Cl)cc4F)n3)CC2)nc2ncc(C(=O)O)c(C)c21] | 2-[(4-{6-[(4-chloro-2- fluorobenzyl)oxy]pyridin- 2-yl}piperidin-1- yl)methyl]-1-(2- methoxyethyl)-7-methyl-1H- imidazo[4,5-b]pyridine-6- carboxylic acid |
| 209B | 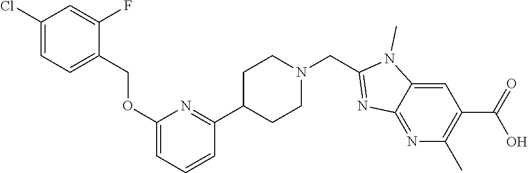 [SMILES: Cc1nc2nc(CN3CCC(c4cccc(OCc5ccc(Cl)cc5F)n4)CC3)n(C)c2cc1C(=O)O] | 2-[(4-{6-[(4-chloro-2- fluorobenzyl)oxy]pyridin- 2-yl}piperidin-1- yl)methyl]-1,5-dimethyl- 1H-imidazo[4,5- b]pyridine-6- carboxylic acid |
| 210B | 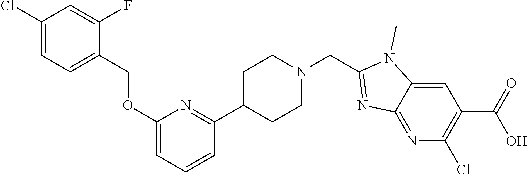 [SMILES: Cn1c(CN2CCC(c3cccc(OCc4ccc(Cl)cc4F)n3)CC2)nc2nc(Cl)c(C(=O)O)cc21] | 5-chloro-2-[(4-{6-[(4-chloro-2- fluorobenzyl)oxy]pyridin-2- yl}piperidin-1- yl)methyl]-1-methyl-1H- benzimidazole-6- carboxylic acid |
| 211B | 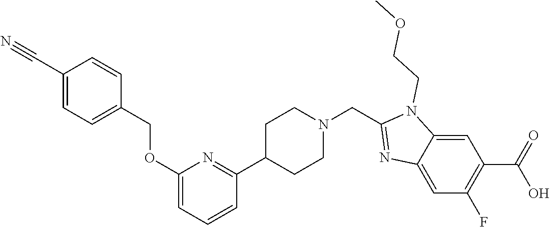 [SMILES: COCCn1c(CN2CCC(c3cccc(OCc4ccc(C#N)cc4)n3)CC2)nc2cc(F)c(C(=O)O)cc21] | 2-[(4-{6-[(4-cyanobenzyl) oxy]pyridin-2- yl}piperidin-1-yl)methyl]- 5-fluoro-1-(2- methoxyethyl)-1H- benzimidazole-6- carboxylic acid |
| 212B | 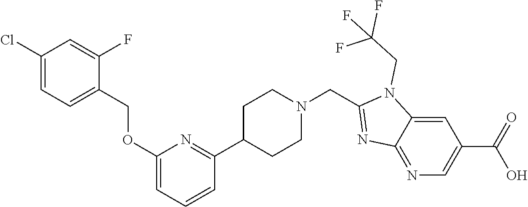 [SMILES: O=C(O)c1cnc2nc(CN3CCC(c4cccc(OCc5ccc(Cl)cc5F)n4)CC3)n(CC(F)(F)F)c2c1] | 2-[(4-{6-[(4-chloro-2- fluorobenzyl)oxy]pyridin- 2-yl}piperidin-1- yl)methyl]-1-(2,2,2- trifluoroethyl)-1H- imidazo[4,5-b]pyridine- 6-carboxylic acid |
| 213B | 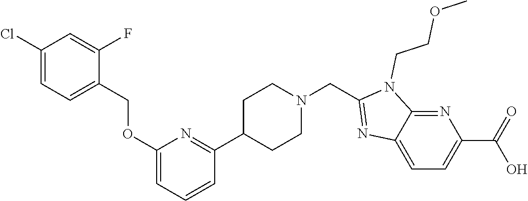 [SMILES: COCCn1c(CN2CCC(c3cccc(OCc4ccc(Cl)cc4F)n3)CC2)nc2ccc(C(=O)O)nc21] | 2-[(4-{6-[(4-chloro-2- fluorobenzyl)oxy]pyridin- 2-yl}piperidin-1- yl)methyl]-3-(2- methoxyethyl)-3H- imidazo[4,5-b]pyridine- 5-carboxylic acid |
| 214B | 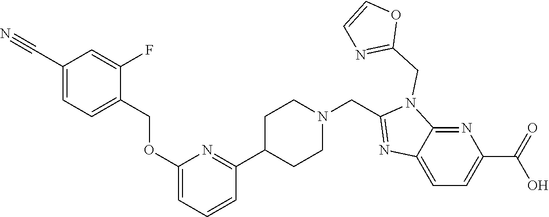 [SMILES: N#Cc1ccc(COc2cccc(C3CCN(Cc4nc5ccc(C(=O)O)nc5n4Cc4ncco4)CC3)n2)c(F)c1] | 2-[(4-{6-[(4-cyano-2- fluorobenzyl)oxy]pyridin- 2-yl}piperidin-1- yl)methyl]-3-(1,3-oxazol- 2-ylmethyl)-3H- imidazo[4,5-b]pyridine- 5-carboxylic acid |
| 215B | 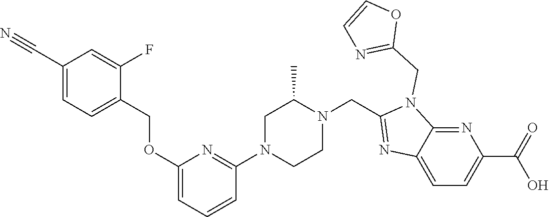 [SMILES: C[C@H]1CN(c2cccc(OCc3ccc(C#N)cc3F)n2)CCN1Cc1nc2ccc(C(=O)O)nc2n1Cc1ncco1] | 2-{[(2S)-4-{6-[(4-cyano-2- fluorobenzyl)oxy]pyridin-2- yl}-2-methylpiperazin-1- yl]methyl}-3-(1,3-oxazol- 2-ylmethyl)-3H-imidazo [4,5-b]pyridine-5- carboxylic acid |
| 216B | 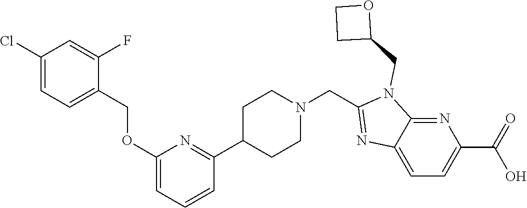 [SMILES: O=C(O)c1ccc2nc(CN3CCC(c4cccc(OCc5ccc(Cl)cc5F)n4)CC3)n(C[C@H]3CCO3)c2n1] | 2-[(4-{6-[(4-chloro-2- fluorobenzyl)oxy]pyridin- 2-yl}piperidin-1-yl)methyl]- 3-[(2R)-oxetan-2-ylmethyl]- 3H-imidazo[4,5-b]pyridine- 5-carboxylic acid |
| 217B | 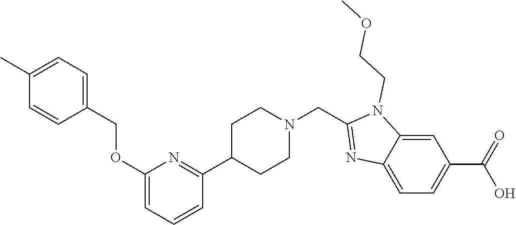 [SMILES: COCCn1c(CN2CCC(c3cccc(OCc4ccc(C)cc4)n3)CC2)nc2ccc(C(=O)O)cc21] | Ammonium 2-((4-(6-((4- Methylbenzyl)oxy)pyridin- 2-yl)piperidin-1- yl)methyl)-1-(2- methoxyethyl)-1H- benzo[d]imidazole-6- carboxylate |
| 218B | 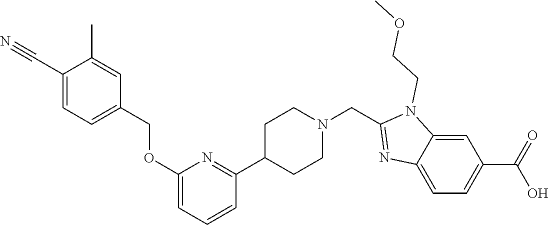 [SMILES: COCCn1c(CN2CCC(c3cccc(OCc4ccc(C#N)c(C)c4)n3)CC2)nc2ccc(C(=O)O)cc21] | 2-((4-(6-((4-cyano-3- methylbenzyl)oxy)pyridin- 2-yl)piperidin-1- yl)methyl)-1-(2- methoxyethyl)-1H- benzo[d]imidazole-6- carboxylic acid |
| 219B | 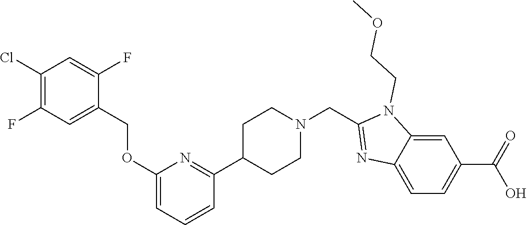 [SMILES: COCCn1c(CN2CCC(c3cccc(OCc4cc(F)c(Cl)cc4F)n3)CC2)nc2ccc(C(=O)O)cc21] | 2-((4-(6-((4-chloro-2,5- difluorobenzyl)oxy) pyridin-2-yl)piperidin-1- yl)methyl)-1-(2- methoxyethyl)-1H- benzo[d]imidazole-6- carboxylic acid |
| 220B | 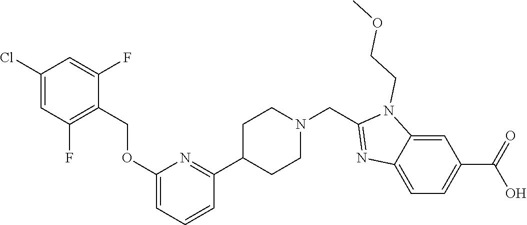 [SMILES: COCCn1c(CN2CCC(c3cccc(OCc4c(F)cc(Cl)cc4F)n3)CC2)nc2ccc(C(=O)O)cc21] | 2-((4-(6-((4-chloro-2,6- difluorobenzyl)oxy) pyridin-2-yl)piperidin-1- yl)methyl)-1-(2- methoxyethyl)-1H- benzo[d]imidazole-6- carboxylic acid |
| 221B | 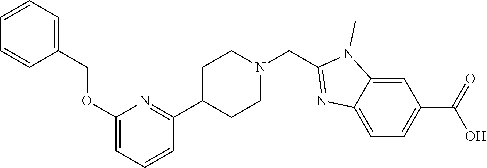 [SMILES: Cn1c(CN2CCC(c3cccc(OCc4ccccc4)n3)CC2)nc2ccc(C(=O)O)cc21] | 2-((4-(6-(Benzyloxy) pyridin-2-yl)piperidin- 1-yl)methyl)-1-methyl- 1H-benzo[d]imidazole- 6-carboxylic acid |
| 222B | 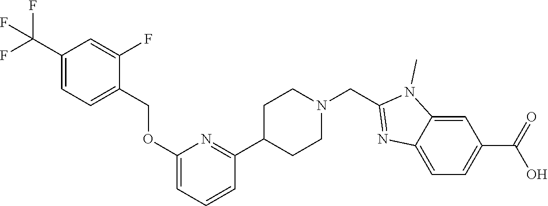 [SMILES: Cn1c(CN2CCC(c3cccc(OCc4ccc(C(F)(F)F)cc4F)n3)CC2)nc2ccc(C(=O)O)cc21] | 2-{[4-(6-{[2-fluoro-4- (trifluoromethyl)benzyl] oxy}pyridin-2-yl)piperidin- 1-yl]methyl}-1-methyl-1H- benzimidazole-6- carboxylic acid |
| 223B | 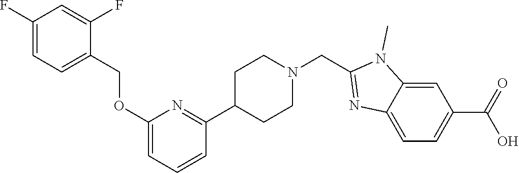 [SMILES: Cn1c(CN2CCC(c3cccc(OCc4ccc(F)cc4F)n3)CC2)nc2ccc(C(=O)O)cc21] | 2-[(4-{6-[(2,4-difluorobenzyl) oxy]pyridin-2-yl}piperidin-1- yl)methyl]-1-methyl-1H- benzimidazole-6-carboxylic acid |
| 224B | 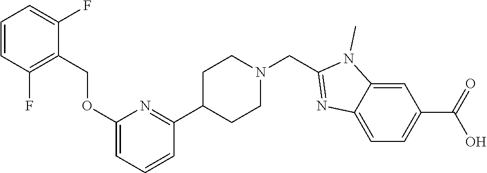 [SMILES: Cn1c(CN2CCC(c3cccc(OCc4c(F)cccc4F)n3)CC2)nc2ccc(C(=O)O)cc21] | 2-[(4-{6-[(2,6-difluorobenzyl) oxy]pyridin-2-yl}piperidin-1- yl)methyl]-1-methyl-1H- benzimidazole-6- carboxylic acid |
| 225B | 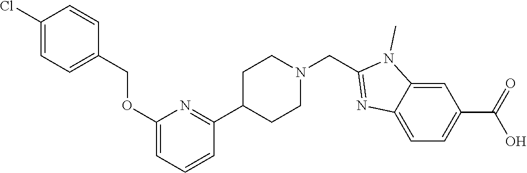 [SMILES: Cn1c(CN2CCC(c3cccc(OCc4ccc(Cl)cc4)n3)CC2)nc2ccc(C(=O)O)cc21] | 2-[(4-{6-[(4-chlorobenzyl) oxy]pyridin-2-yl}piperidin-1- yl)methyl]-1-methyl-1H- benzimidazole-6- carboxylic acid |
| 226B | 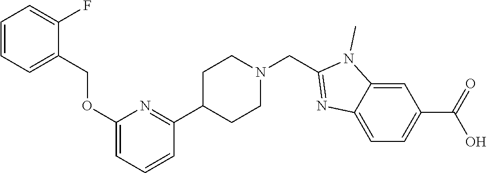 [SMILES: Cn1c(CN2CCC(c3cccc(OCc4ccccc4F)n3)CC2)nc2ccc(C(=O)O)cc21] | 2-[(4-{6-[(2-fluorobenzyl) oxy]pyridin-2-yl}piperidin- 1-yl)methyl]-1-methyl-1H- benzimidazole-6- carboxylic acid |
| 227B | 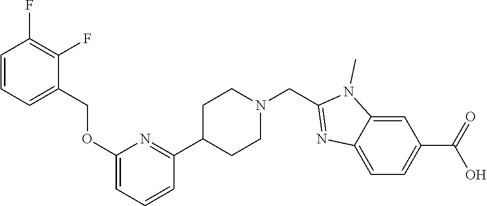 [SMILES: Cn1c(CN2CCC(c3cccc(OCc4cccc(F)c4F)n3)CC2)nc2ccc(C(=O)O)cc21] | 2-[(4-{6-[(2,3-difluorobenzyl) oxy]pyridin-2-yl}piperidin-1- yl)methyl]-1-methyl-1H- benzimidazole-6-carboxylic acid |
| 228B | 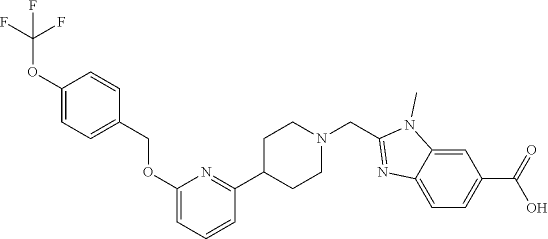 [SMILES: Cn1c(CN2CCC(c3cccc(OCc4ccc(OC(F)(F)F)cc4)n3)CC2)nc2ccc(C(=O)O)cc21] | 1-methyl-2-{[4-(6-{[4- (trifluoromethoxy)benzyl] oxy}pyridin-2-yl)piperidin- 1-yl]methyl}-1H- benzimidazole- 6-carboxylic acid |
| 229B | 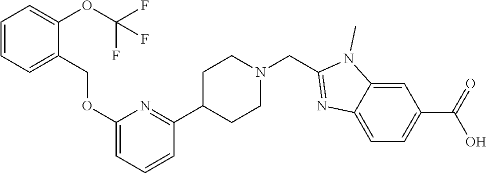 [SMILES: Cn1c(CN2CCC(c3cccc(OCc4ccccc4OC(F)(F)F)n3)CC2)nc2ccc(C(=O)O)cc21] | 1-methyl-2-{[4-(6-{[2- (trifluoromethoxy)benzyl] oxy}pyridin-2-yl) piperidin- 1-yl]methyl}-1H- benzimidazole- 6-carboxylic acid |
| 230B | 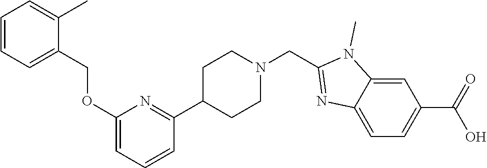 [SMILES: Cc1ccccc1COc1cccc(C2CCN(Cc3nc4ccc(C(=O)O)cc4n3C)CC2)n1] | 1-methyl-2-[(4-{6-[(2- methylbenzyl)oxy]pyridin- 2-yl}piperidin-1-yl)methyl]- 1H-benzimidazole-6- carboxylic acid |
| 231B | 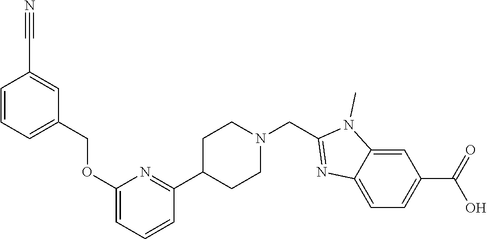 [SMILES: Cn1c(CN2CCC(c3cccc(OCc4cccc(C#N)c4)n3)CC2)nc2ccc(C(=O)O)cc21] | 2-[(4-{6-[(3-cyanobenzyl) oxy]pyridin-2-yl}piperidin- 1-yl)methyl]-1-methyl-1H- benzimidazole-6- carboxylic acid |
| 232B | 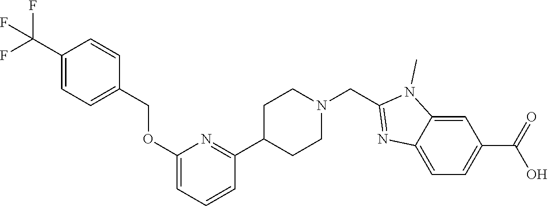 [SMILES: Cn1c(CN2CCC(c3cccc(OCc4ccc(C(F)(F)F)cc4)n3)CC2)nc2ccc(C(=O)O)cc21] | 1-methyl-2-{[4-(6-{[4- (trifluoromethyl)benzyl] oxy}pyridin-2-yl)piperidin- 1-yl]methyl}-1H- benzimidazole- 6-carboxylic acid |
| 233B | 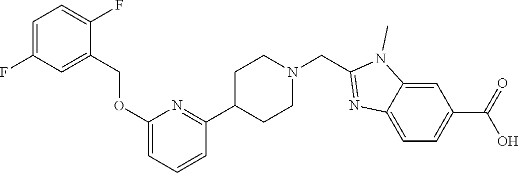 [SMILES: Cn1c(CN2CCC(c3cccc(OCc4cc(F)ccc4F)n3)CC2)nc2ccc(C(=O)O)cc21] | 2-[(4-{6-[(2,5-difluorobenzyl) oxy]pyridin-2-yl}piperidin-1- yl)methyl]-1-methyl-1H- benzimidazole-6- carboxylic acid |
| 234B | 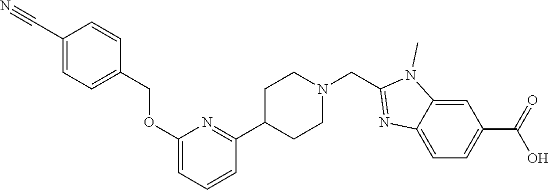 [SMILES: Cn1c(CN2CCC(c3cccc(OCc4ccc(C#N)cc4)n3)CC2)nc2ccc(C(=O)O)cc21] | 2-[(4-{6-[(4-cyanobenzyl) oxy]pyridin-2-yl}piperidin-1- yl)methyl]-1-methyl-1H- benzimidazole-6- carboxylic acid |
| 235B | 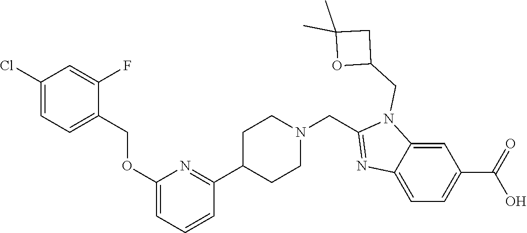 [SMILES: CC1(C)CC(Cn2c(CN3CCC(c4cccc(OCc5ccc(Cl)cc5F)n4)CC3)nc3ccc(C(=O)O)cc32)O1] | 2-[(4-{6-[(4-chloro-2- fluorobenzyl)oxy]pyridin-2- yl}piperidin-1-yl)methyl]-1- [(4,4-dimethyloxetan-2- yl)methyl]-1H-benzimidazole- 6-carboxylic acid |
| 236B | 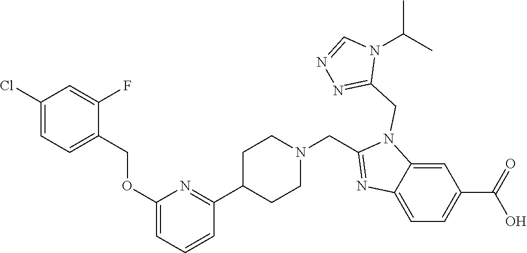 [SMILES: CC(C)n1cnnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-{[4-(propan-2-yl)-4H-1,2,4- triazol-3-yl]methyl}-1H- benzimidazole-6- carboxylic acid |
| 237B | 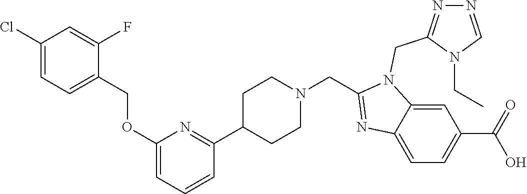 [SMILES: CCn1cnnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-[(4-ethyl-4H-1,2,4-triazol-3- yl)methyl]-1H-benzimidazole- 6-carboxylic acid |
| 238B | 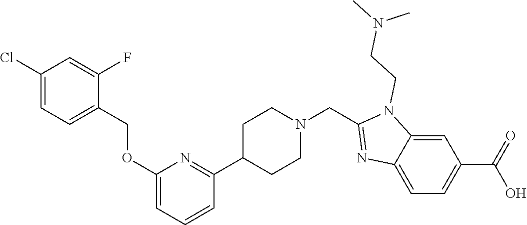 [SMILES: CN(C)CCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-[2-(dimethylamino)ethyl]-1H- benzimidazole-6-carboxylic acid |
| 239B | 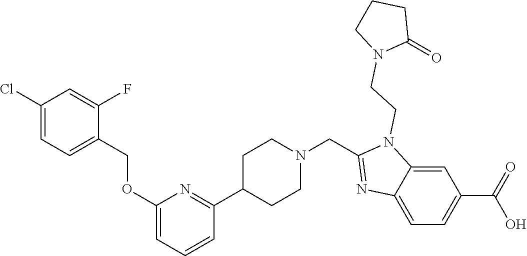 [SMILES: O=C(O)c1ccc2nc(CN3CCC(c4cccc(OCc5ccc(Cl)cc5F)n4)CC3)n(CCN3CCCC3=O)c2c1] | 2-[(4-{6-[(4-chloro-2- fluorobenzyl)oxy]pyridin- 2-yl} piperidin-1-yl)methyl]- 1-[2-(2-oxopyrrolidin-1- yl)ethyl]-1H-benzimidazole- 6-carboxylic acid |
| 240B | 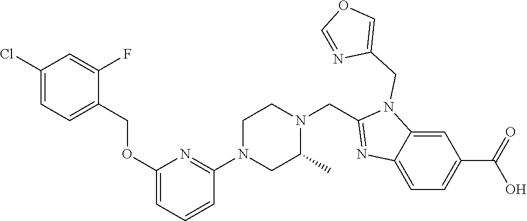 [SMILES: C[C@@H]1CN(c2cccc(OCc3ccc(Cl)cc3F)n2)CCN1Cc1nc2ccc(C(=O)O)cc2n1Cc1cocn1] | 2-{[(2S)-4-{6-[(4-chloro-2- fluorobenzyl)oxy]pyridin- 2-yl}-2-methylpiperazin- 1-yl]methyl}-1-(1,3-oxazol- 4-ylmethyl)-1H- benzimidazole-6-carboxylic acid |
| 241B | 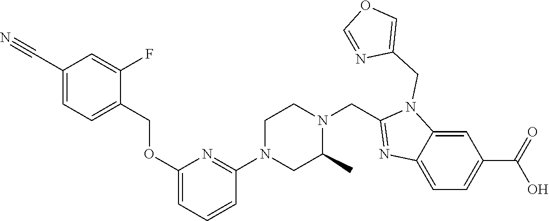 [SMILES: C[C@H]1CN(c2cccc(OCc3ccc(C#N)cc3F)n2)CCN1Cc1nc2ccc(C(=O)O)cc2n1Cc1cocn1] | 2-{[(2S)-4-{6-[(4-cyano-2- fluorobenzyl)oxy]pyridin-2- yl}-2-methylpiperazin-1- yl]methyl}-1-(1,3-oxazol- 4-ylmethyl)-1H- benzimidazole-6-carboxylic acid |
| 242B | 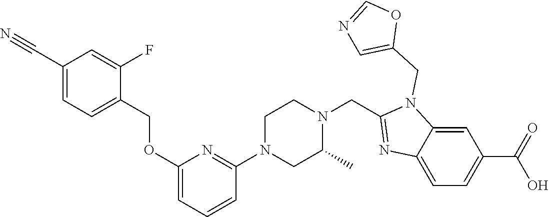 [SMILES: C[C@@H]1CN(c2cccc(OCc3ccc(C#N)cc3F)n2)CCN1Cc1nc2ccc(C(=O)O)cc2n1Cc1cnco1] | 2-{[(2S)-4-{6-[(4-cyano-2- fluorobenzyl)oxy]pyridin- 2-yl}-2-methylpiperazin-1- yl]methyl}-1-(1,3-oxazol- 5-ylmethyl)-1H-benzimidazole- 6-carboxylic acid |
| 243B | 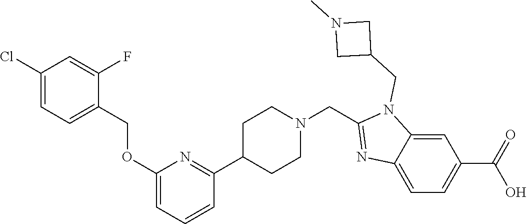 [SMILES: CN1CC(Cn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin- 2-yl}piperidin-1-yl)methyl]- 1-[(1-methylazetidin-3- yl)methyl]-1H-benzimidazole- 6-carboxylic acid |
| 244B | 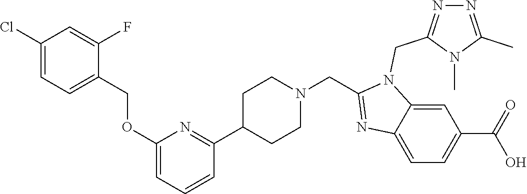 [SMILES: Cc1nnc(Cn2c(CN3CCC(c4cccc(OCc5ccc(Cl)cc5F)n4)CC3)nc3ccc(C(=O)O)cc32)n1C] | 2-[(4-{6-[(4-chloro-2- fluorobenzyl)oxy]pyridin- 2-yl}piperidin-1-yl)methyl]- 1-[(4,5-dimethyl-4H-1,2,4- triazol-3-yl)methyl]-1H- benzimidazole-6- carboxylic acid |
| 245B | 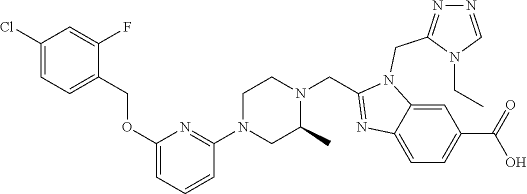 [SMILES: CCn1cnnc1Cn1c(CN2CCN(c3cccc(OCc4ccc(Cl)cc4F)n3)C[C@@H]2C)nc2ccc(C(=O)O)cc21] | 2-{[(2S)-4-{6-[(4-chloro-2- fluorobenzyl)oxy]pyridin-2- yl}-2-methylpiperazin-1- yl]methyl}-1-[(4-ethyl- 4H-1,2,4-triazol-3-yl)methyl]- 1H-benzimidazole-6- carboxylic acid |
| 246B | 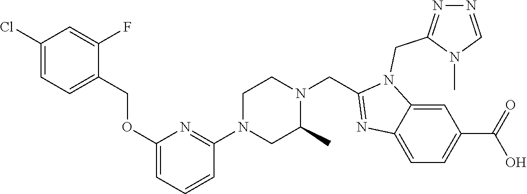 [SMILES: C[C@H]1CN(c2cccc(OCc3ccc(Cl)cc3F)n2)CCN1Cc1nc2ccc(C(=O)O)cc2n1Cc1nncn1C] | 2-{[(2S)-4-{6-[(4-chloro-2- fluorobenzyl)oxy]pyridin-2- yl}-2-methylpiperazin-1- yl]methyl}-1-[(1-methyl- 1H-1,2,3-triazol-5- yl)methyl]-1H-benzimidazole- 6-carboxylic acid |
| 247B | 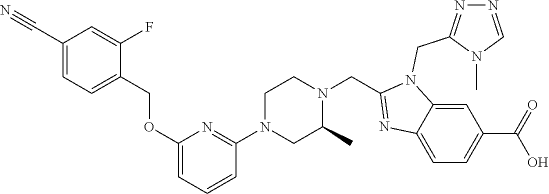 [SMILES: C[C@H]1CN(c2cccc(OCc3ccc(C#N)cc3F)n2)CCN1Cc1nc2ccc(C(=O)O)cc2n1Cc1nncn1C] | 2-{[(2S)-4-{6-[(4-cyano-2- fluorobenzyl)oxy]pyridin-2-yl}-2- methylpiperazin-1-yl]methyl}-1- [(1-methyl-1H-1,2,3-triazol-5- yl)methyl]-1H-benzimidazole- 6-carboxylic acid |
| 248B | 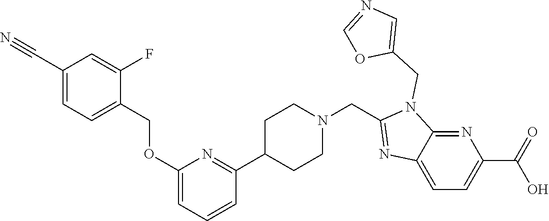 [SMILES: N#Cc1ccc(COc2cccc(C3CCN(Cc4nc5ccc(C(=O)O)nc5n4Cc4cnco4)CC3)n2)c(F)c1] | 2-[(4-{6-[(4-cyano-2- fluorobenzyl)oxy]pyridin-2- yl}piperidin-1-yl)methyl]-3- (1,3-oxazol-5-ylmethyl)-3H- imidazo[4,5-b]pyridine-5- carboxylic acid |
| 249B | 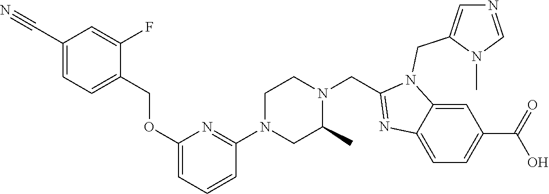 [SMILES: C[C@H]1CN(c2cccc(OCc3ccc(C#N)cc3F)n2)CCN1Cc1nc2ccc(C(=O)O)cc2n1Cc1cncn1C] | 2-{[(2S)-4-{6-[(4-cyano-2- fluorobenzyl)oxy]pyridin-2- yl}-2-methylpiperazin-1- yl]methyl}-1-[(1-methyl- 1H-imidazol-5-yl)methyl]- 1H-benzimidazole-6- carboxylic acid |
| 251B | 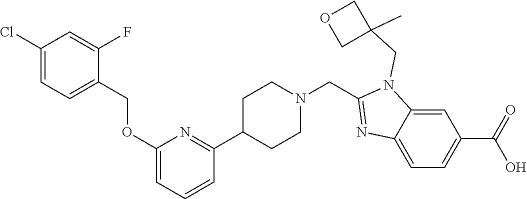 [SMILES: CC1(Cn2c(CN3CCC(c4cccc(OCc5ccc(Cl)cc5F)n4)CC3)nc3ccc(C(=O)O)cc32)COC1] | 2-[(4-{6-[(4-chloro-2- fluorobenzyl)oxy]pyridin- 2-yl}piperidin-1- yl)methyl]-1-[(3- methyloxetan-3-yl)methyl]- 1H-benzimidazole-6- carboxylic acid |
| 252B | 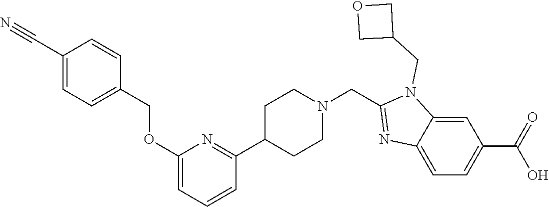 [SMILES: N#Cc1ccc(COc2cccc(C3CCN(Cc4nc5ccc(C(=O)O)cc5n4CC4COC4)CC3)n2)cc1] | 2-[(4-{6-[(4- cyanobenzyl)oxy]pyridin-2- yl}piperidin-1-yl)methyl]-1- (oxetan-3-ylmethyl)-1H- benzimidazole-6-carboxylic acid |
| 253B | 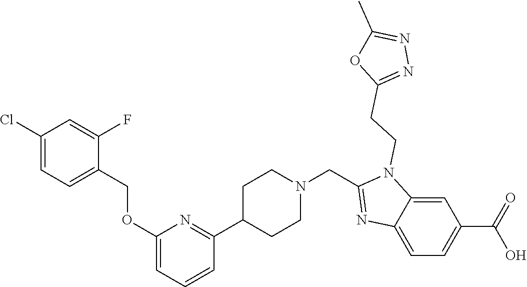 [SMILES: Cc1nnc(CCn2c(CN3CCC(c4cccc(OCc5ccc(Cl)cc5F)n4)CC3)nc3ccc(C(=O)O)cc32)o1] | 2-[(4-{6-[(4-chloro-2- fluorobenzyl)oxy]pyridin-2- yl}piperidin-1-yl)methyl]-1- [2-(5-methyl-1,3,4-oxadiazol- 2-yl)ethyl]-1H-benzimidazole- 6-carboxylic acid |
| 254B | 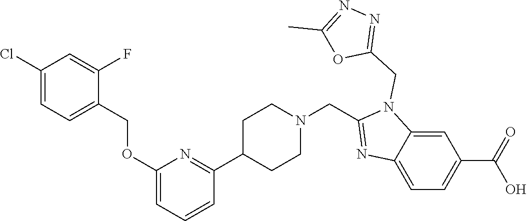 [SMILES: Cc1nnc(Cn2c(CN3CCC(c4cccc(OCc5ccc(Cl)cc5F)n4)CC3)nc3ccc(C(=O)O)cc32)o1] | 2-[(4-{6-[(4-chloro-2- fluorobenzyl)oxy]pyridin- 2-yl}piperidin-1-yl)methyl]- 1-[(5-methyl-1,3,4-oxadiazol- 2-yl)methyl]-1H- benzimidazole-6-carboxylic acid |
| 255B | 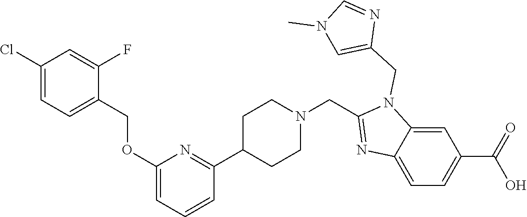 [SMILES: Cn1cnc(Cn2c(CN3CCC(c4cccc(OCc5ccc(Cl)cc5F)n4)CC3)nc3ccc(C(=O)O)cc32)c1] | 2-[(4-{6-[(4-chloro-2- fluorobenzyl)oxy]pyridin- 2-yl} piperidin-1- yl)methyl]-1-[(1-methyl- 1H-imidazol-4- yl)methyl]-1H- benzimidazole-6- carboxylic acid |
| 256B | 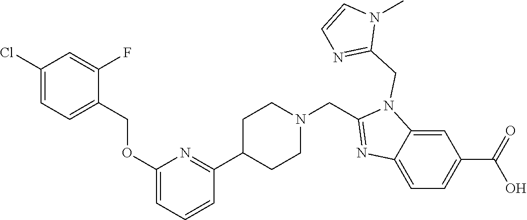 [SMILES: Cn1ccnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[(1-methyl- 1H-imidazol-2- yl)methyl]-1H- benzimidazole-6- carboxylic acid |
| 257B | 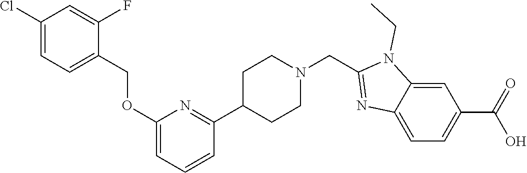 [SMILES: CCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-ethyl-1H- benzimidazole-6- carboxylic acid |
| 258B | 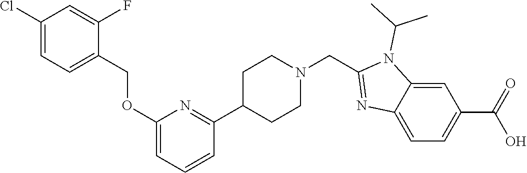 [SMILES: CC(C)n1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-(propan-2- yl)-1H-benzimidazole- 6-carboxylic acid |
| 259B | 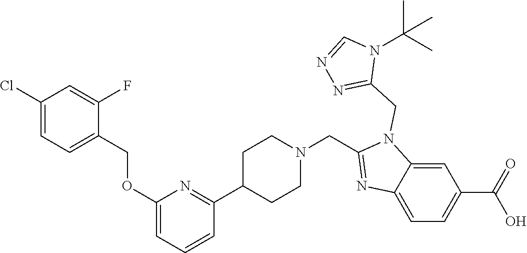 [SMILES: CC(C)(C)n1cnnc1Cn1c(CN2CCC(c3cccc(OCc4ccc(Cl)cc4F)n3)CC2)nc2ccc(C(=O)O)cc21] | 1-[(4-tert-butyl-4H-1,2,4- triazol-3-yl)methyl]-2-[(4- {6-[(4-chloro-2- fluorobenzyl)oxy]pyridin- 2-yl}piperidin-1- yl)methyl]-1H- benzimidazole-6-carboxylic acid |
| 260B | 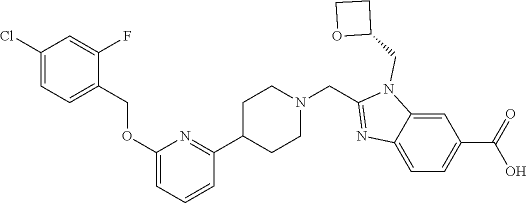 [SMILES: O=C(O)c1ccc2nc(CN3CCC(c4cccc(OCc5ccc(Cl)cc5F)n4)CC3)n(C[C@H]3CCO3)c2c1] | 2-[(4-{6-[(4-chloro-2- fluorobenzyl)oxy]pyridin- 2-yl}piperazin-1- yl)methyl]-1-[(2R)-oxetan- 2-ylmethyl]-1H- benzimidazole-6- carboxylic acid |
| 261B | 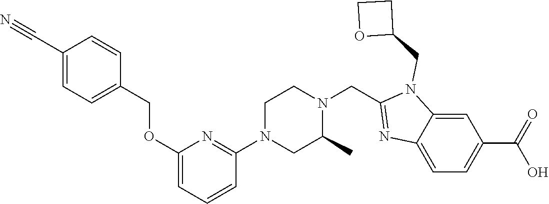 [SMILES: C[C@H]1CN(c2cccc(OCc3ccc(C#N)cc3)n2)CCN1Cc1nc2ccc(C(=O)O)cc2n1C[C@@H]1CCO1] | 2-{[(2S)-4-{6-[(4- cyanobenzyl)oxy]pyridin- 2-yl}-2-methylpiperazin- 1-yl]methyl}-1-[(2S)- oxetan-2-ylmethyl]-1H- benzimidazole-6- carboxylic acid |
| 262B | 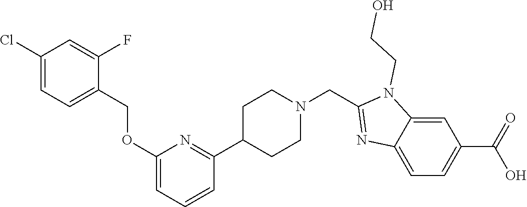 [SMILES: O=C(O)c1ccc2nc(CN3CCC(c4cccc(OCc5ccc(Cl)cc5F)n4)CC3)n(CCO)c2c1] | 2-[(4-{6-[(4-chloro-2- fluorobenzyl)oxy]pyridin- 2-yl} piperidin-1- yl)methyl]-1-(2-hydroxyethyl)- 1H-benzimidazole-6- carboxylic acid |
| 263B | 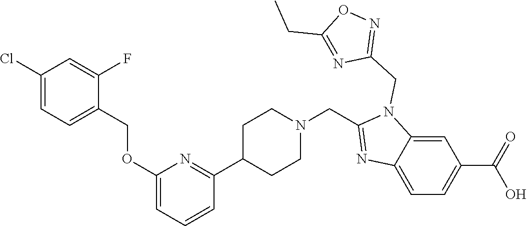 [SMILES: CCc1nc(Cn2c(CN3CCC(c4cccc(OCc5ccc(Cl)cc5F)n4)CC3)nc3ccc(C(=O)O)cc32)no1] | 2-[(4-{6-[(4-chloro-2- fluorobenzyl)oxy]pyridin-2- yl}piperidin-1- yl)methyl]-1-[(5-ethyl-1,2,4- oxadiazol-3-yl)methyl]-1H- benzimidazole-6-carboxylic acid |
| 264B | 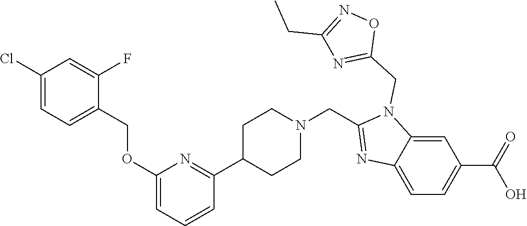 [SMILES: CCc1noc(Cn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin- 2-yl}piperidin-1- yl)methyl]-1-[(3-ethyl- 1,2,4-oxadiazol-5- yl)methyl]-1H-benzimidazole- 6-carboxylic acid |
| 265B | 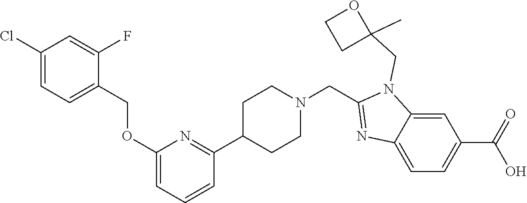 [SMILES: CC1(Cn2c(CN3CCC(c4cccc(OCc5ccc(Cl)cc5F)n4)CC3)nc3ccc(C(=O)O)cc32)CCO1] | 2-[(4-{6-[(4-chloro-2- fluorobenzyl)oxy]pyridin- 2-yl}piperidin-1- yl)methyl]-1-[(2- methyloxetan-2-yl)methyl]- 1H-benzimidazole-6- carboxylic acid |
| 266B | 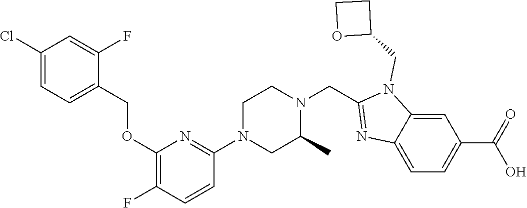 [SMILES: C[C@H]1CN(c2ccc(F)c(OCc3ccc(Cl)cc3F)n2)CCN1Cc1nc2ccc(C(=O)O)cc2n1C[C@H]1CCO1] | 2-{[(2S)-4-{6-[(2,4- difluorobenzyl)oxy]-5- fluoropyridin-2-yl}-2- methylpiperazin-1- yl]methyl}-1-[(2R)-oxetan- 2-ylmethyl]-1H- benzimidazole-6- carboxylic acid |
| 267B | 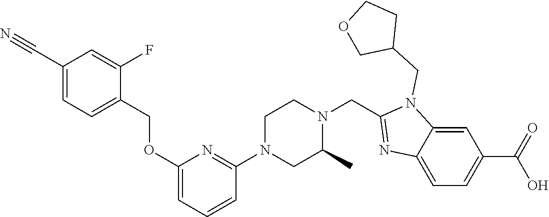 [SMILES: C[C@H]1CN(c2cccc(OCc3ccc(C#N)cc3F)n2)CCN1Cc1nc2ccc(C(=O)O)cc2n1CC1CCOC1] | 2-{[(2S)-4-{6-[(4-cyano-2- fluorobenzyl)oxy]pyridin- 2-yl}-2-methylpiperazin-1- yl]methyl}-1-[(3R)- tetrahydrofuran-3-ylmethyl]- 1H-benzimidazole-6- carboxylic acid |
| 268B | 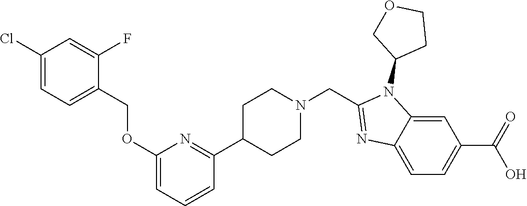 [SMILES: O=C(O)c1ccc2nc(CN3CCC(c4cccc(OCc5ccc(Cl)cc5F)n4)CC3)n([C@@H]3CCOC3)c2c1] | 2-[(4-{6-[(4-chloro-2- fluorobenzyl)oxy]pyridin- 2-yl}piperidin-1-yl)methyl]- 1-[(3R)-tetrahydrofuran-3- yl]-1H-benzimidazole- 6-carboxylic acid |
| 269B | 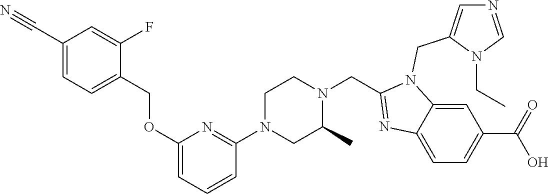 [SMILES: CCn1cncc1Cn1c(CN2CCN(c3cccc(OCc4ccc(C#N)cc4F)n3)C[C@@H]2C)nc2ccc(C(=O)O)cc21] | 2-{[(2S)-4-{6-[(4-cyano-2- fluorobenzyl)oxy]pyridin- 2-yl}-2-methylpiperazin- 1-yl]methyl}-1-[(1-ethyl- 1H-imidazol-5-yl)methyl]- 1H-benzimidazole-6- carboxylic acid |
| 270B | 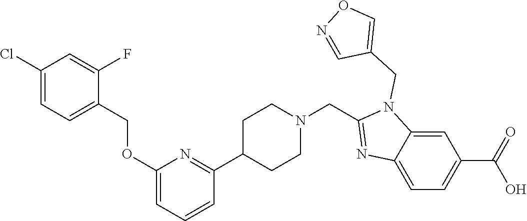 [SMILES: O=C(O)c1ccc2nc(CN3CCC(c4cccc(OCc5ccc(Cl)cc5F)n4)CC3)n(Cc3cnoc3)c2c1] | 2-[(4-{6-[(4-chloro-2- fluorobenzyl)oxy]pyridin- 2-yl}piperidin-1-yl)methyl]- 1-(1,2-oxazol-4-ylmethyl)-1H- benzimidazole-6- carboxylic acid |
| 271B | 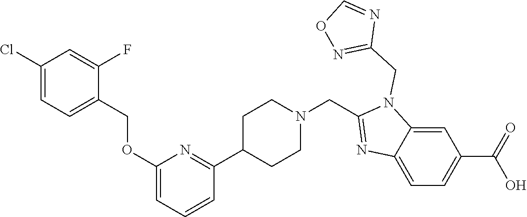 [SMILES: O=C(O)c1ccc2nc(CN3CCC(c4cccc(OCc5ccc(Cl)cc5F)n4)CC3)n(Cc3ncon3)c2c1] | 2-[(4-{6-[(4-chloro-2- fluorobenzyl)oxy]pyridin- 2-yl}piperidin-1- yl)methyl]-1-(1,2,4- oxadiazol-3-ylmethyl)- 1H-benzimidazole-6- carboxylic acid |
| 272B | 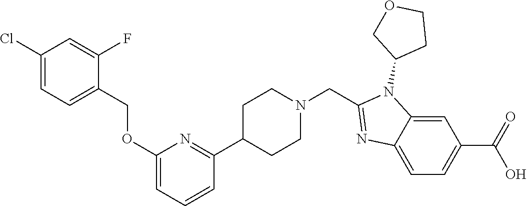 [SMILES: O=C(O)c1ccc2nc(CN3CCC(c4cccc(OCc5ccc(Cl)cc5F)n4)CC3)n([C@H]3CCOC3)c2c1] | 2-[(4-{6-[(4-chloro-2- fluorobenzyl)oxy]pyridin- 2-yl}piperidin-1- yl)methyl]-1-[(3S)- tetrahydrofuran-3-yl]- 1H-benzimidazole-6- carboxylic acid |
| 273B | 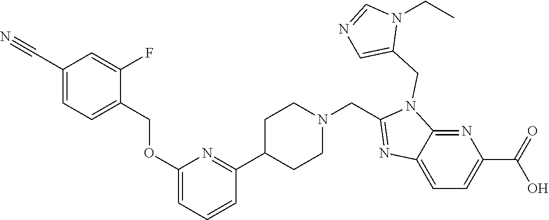 [SMILES: CCn1cncc1Cn1c(CN2CCC(c3cccc(OCc4ccc(C#N)cc4F)n3)CC2)nc2ccc(C(=O)O)nc21] | 2-[(4-{6-[(4-cyano-2- fluorobenzyl)oxy]pyridin- 2-yl}piperidin-1- yl)methyl]-3-[(1-ethyl- 1H-imidazol-5-yl)methyl]- 3H-imidazo[4,5-b]pyridine- 5-carboxylic acid |
| 274B | 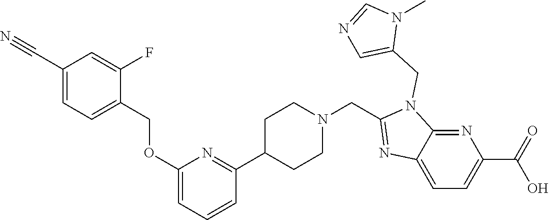 [SMILES: Cn1cncc1Cn1c(CN2CCC(c3cccc(OCc4ccc(C#N)cc4F)n3)CC2)nc2ccc(C(=O)O)nc21] | 2-[(4-{6-[(4-cyano-2- fluorobenzyl)oxy]pyridin- 2-yl}piperidin-1-yl)methyl]- 3-[(1-methyl-1H-imidazol-5- yl)methyl]-3H-imidazo[4,5- b]pyridine-5-carboxylic acid |
| 275B | 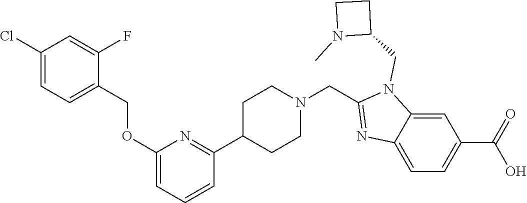 [SMILES: CN1CC[C@@H]1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [(1-methylazetidin-2- yl)methyl]-1H-benzimidazole- 6-carboxylic acid |
| 276B | 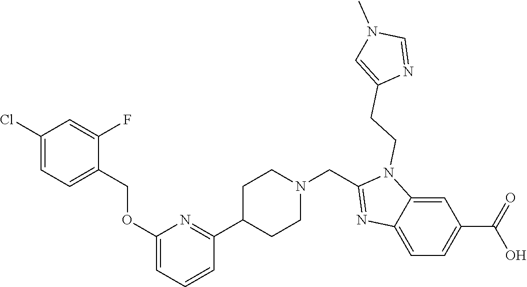 [SMILES: Cn1cnc(CCn2c(CN3CCC(c4cccc(OCc5ccc(Cl)cc5F)n4)CC3)nc3ccc(C(=O)O)cc32)c1] | 2-[(4-{6-[(4-chloro-2- fluorobenzyl)oxy]pyridin- 2-yl}piperidin-1- yl)methyl]-1-[2-(1-methyl- 1H-imidazol-4-yl)ethyl]- 1H-benzimidazole-6- carboxylic acid |
| 277B | 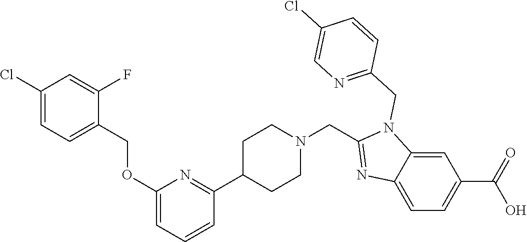 [SMILES: O=C(O)c1ccc2nc(CN3CCC(c4cccc(OCc5ccc(Cl)cc5F)n4)CC3)n(Cc3ccc(Cl)cn3)c2c1] | 2-[(4-{6-[(4-chloro-2- fluorobenzyl)oxy]pyridin- 2-yl} piperidin-1-yl)methyl]- 1-[(5-chloropyridin-2-yl)methyl]- 1H-benzimidazole-6- carboxylic acid |
| 278B | 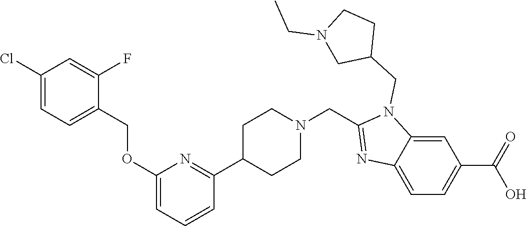 [SMILES: CCN1CCC(Cn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin- 2-yl} piperidin-1- yl)methyl]-1-[(1- ethylpyrrolidin-3-yl)methyl]- 1H-benzimidazole-6- carboxylic acid |
| 279B | 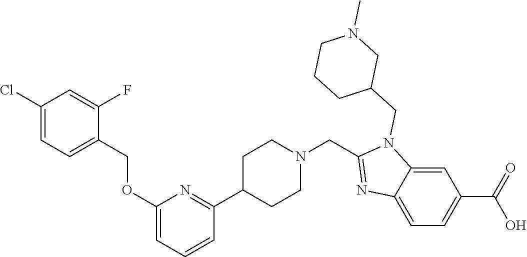 [SMILES: CN1CCCC(Cn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin- 2-yl} piperidin-1-yl)methyl]- 1-[(1-methylpiperidin-3- yl)methyl]-1H-benzimidazole- 6-carboxylic acid |
| 280B | 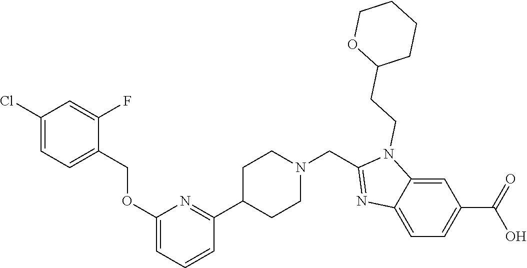 [SMILES: O=C(O)c1ccc2nc(CN3CCC(c4cccc(OCc5ccc(Cl)cc5F)n4)CC3)n(CCC3CCCCO3)c2c1] | 2-[(4-{6-[(4-chloro-2- fluorobenzyl)oxy]pyridin- 2-yl}piperidin-1-yl)methyl]- 1-[2-(tetrahydro-2H-pyran-2- yl)ethyl]-1H-benzimidazole- 6-carboxylic acid |
| 281B | 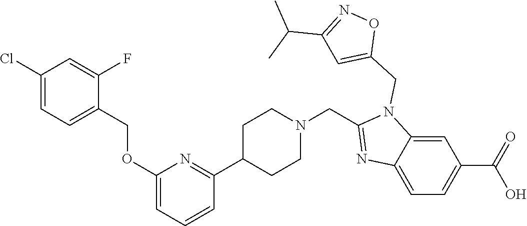 [SMILES: CC(C)c1cc(Cn2c(CN3CCC(c4cccc(OCc5ccc(Cl)cc5F)n4)CC3)nc3ccc(C(=O)O)cc32)on1] | 2-[(4-{6-[(4-chloro-2- fluorobenzyl)oxy]pyridin-2- yl}piperidin-1-yl)methyl]-1- {[3-(propan-2-yl)-1,2-oxazol-5- yl]methyl}-1H-benzimidazole- 6-carboxylic acid |
| 282B | 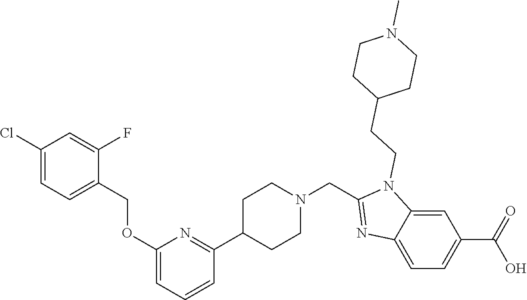 [SMILES: CN1CCC(CCn2c(CN3CCC(c4cccc(OCc5ccc(Cl)cc5F)n4)CC3)nc3ccc(C(=O)O)cc32)CC1] | 2-[(4-{6-[(4-chloro-2- fluorobenzyl)oxy]pyridin-2- yl}piperidin-1-yl)methyl]-1- [2-(1-methylpiperidin-4- yl)ethyl]-1H-benzimidazole- 6-carboxylic acid |
| 283B | 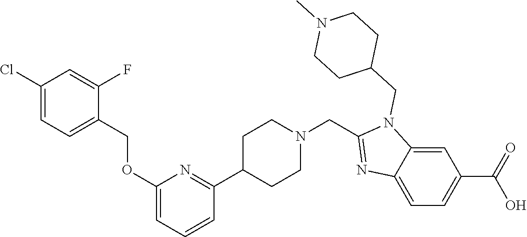 [SMILES: CN1CCC(Cn2c(CN3CCC(c4cccc(OCc5ccc(Cl)cc5F)n4)CC3)nc3ccc(C(=O)O)cc32)CC1] | 2-[(4-{6-[(4-chloro-2- fluorobenzyl)oxy]pyridin- 2-yl}piperidin-1- yl)methyl]-1-[(1- methylpiperidin-4- yl)methyl]-1H- benzimidazole-6- carboxylic acid |
| 284B | 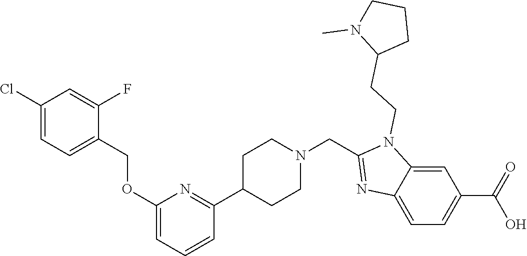 [SMILES: CN1CCCC1CCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[2-(1- methylpyrrolidin-2- yl)ethyl]-1H-benzimidazole- 6-carboxylic acid |
| 285B | 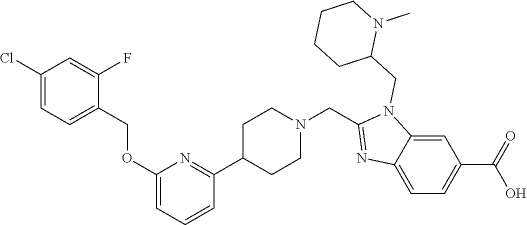 [SMILES: CN1CCCCC1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-[(1-methylpiperidin-2- yl)methyl]-1H-benzimidazole- 6-carboxylic acid |
| 286B | 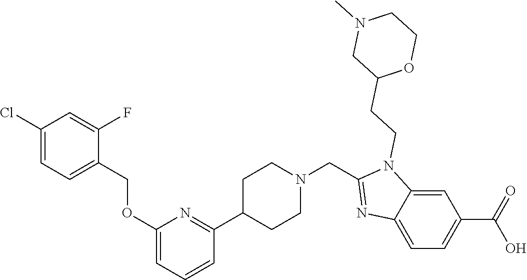 [SMILES: CN1CCOC(CCn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin-2- yl}piperidin-1-yl)methyl]-1- [2-(4-methylmorpholin-2- yl)ethyl]-1H-benzimidazole- 6-carboxylic acid |
| 287B | 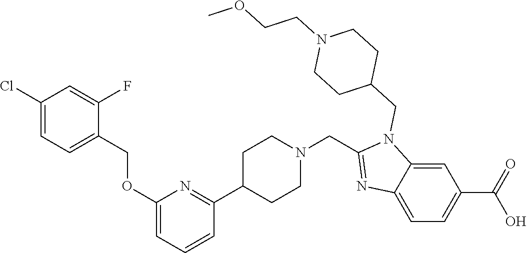 [SMILES: COCCN1CCC(Cn2c(CN3CCC(c4cccc(OCc5ccc(Cl)cc5F)n4)CC3)nc3ccc(C(=O)O)cc32)CC1] | 2-[(4-{6-[(4-chloro-2- fluorobenzyl)oxy]pyridin-2- yl}piperidin-1-yl)methyl]- 1-{[1-(2-methoxyethyl) piperidin-4-yl]methyl}-1H- benzimidazole-6- carboxylic acid |
| 288B | 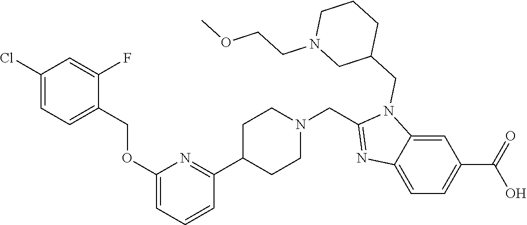 [SMILES: COCCN1CCCC(Cn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin- 2-yl} piperidin-1- yl)methyl]-1-{[1-(2- methoxyethyl)piperidin- 3-yl]methyl}-1H- benzimidazole-6- carboxylic acid |
| 289B | 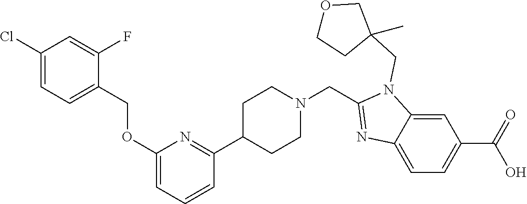 [SMILES: CC1(Cn2c(CN3CCC(c4cccc(OCc5ccc(Cl)cc5F)n4)CC3)nc3ccc(C(=O)O)cc32)CCOC1] | 2-[(4-{6-[(4-chloro-2- fluorobenzyl)oxy]pyridin- 2-yl}piperidin-1-yl)methyl]- 1-[(3-methyltetrahydrofuran- 3-yl)methyl]-1H- benzimidazole-6-carboxylic acid |
| 290B | 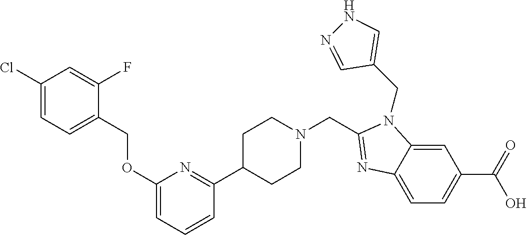 [SMILES: O=C(O)c1ccc2nc(CN3CCC(c4cccc(OCc5ccc(Cl)cc5F)n4)CC3)n(Cc3cn[nH]c3)c2c1] | 2-[(4-{6-[(4-chloro-2- fluorobenzyl)oxy]pyridin-2- yl}piperidin-1-yl)methyl]- 1-(1H-pyrazol-4-ylmethyl)- 1H-benzimidazole-6- carboxylic acid |
| 291B | 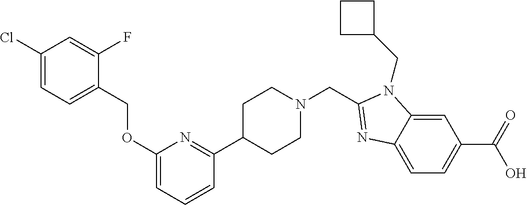 [SMILES: O=C(O)c1ccc2nc(CN3CCC(c4cccc(OCc5ccc(Cl)cc5F)n4)CC3)n(CC3CCC3)c2c1] | 2-[(4-{6-[(4-chloro-2- fluorobenzyl)oxy]pyridin- 2-yl}piperidin-1-yl)methyl]- 1-(cyclobutylmethyl)-1H- benzimidazole-6- carboxylic acid |
| 292B | 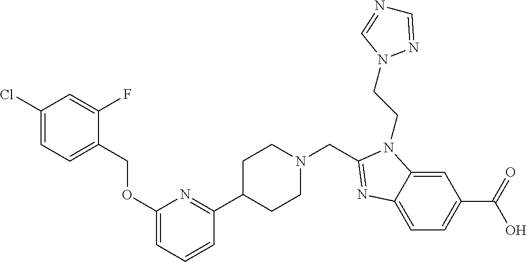 [SMILES: O=C(O)c1ccc2nc(CN3CCC(c4cccc(OCc5ccc(Cl)cc5F)n4)CC3)n(CCn3cncn3)c2c1] | 2-[(4-{6-[(4-chloro-2- fluorobenzyl)oxy]pyridin-2- yl}piperidin-1-yl)methyl]-1- [2-(1H-1,2,4-triazol-1-yl) ethyl]-1H-benzimidazole- 6-carboxylic acid |
| 293B | 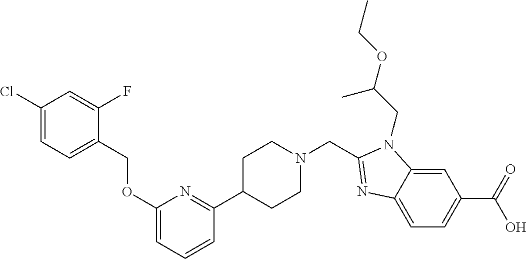 [SMILES: CCOC(C)Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-(2-ethoxypropyl)-1H- benzimidazole-6- carboxylic acid |
| 294B | 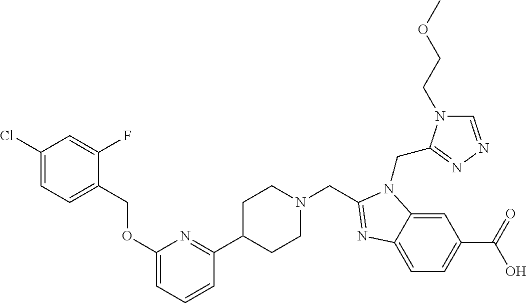 [SMILES: COCCn1cnnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl} piperidin-1-yl)methyl]- 1-{[4-(2-methoxyethyl)-4H- 1,2,4-triazol-3-yl]methyl}- 1H-benzimidazole-6- carboxylic acid |
| 295B | 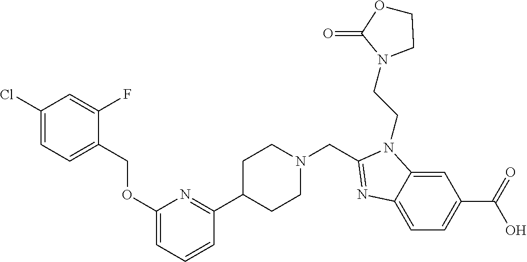 [SMILES: O=C(O)c1ccc2nc(CN3CCC(c4cccc(OCc5ccc(Cl)cc5F)n4)CC3)n(CCN3CCOC3=O)c2c1] | 2-[(4-{6-[(4-chloro-2- fluorobenzyl)oxy]pyridin-2- yl}piperidin-1-yl)methyl]- 1-[2-(2-oxo-1,3-oxazolidin-3- yl)ethyl]-1H-benzimidazole- 6-carboxylic acid |
| 296B | 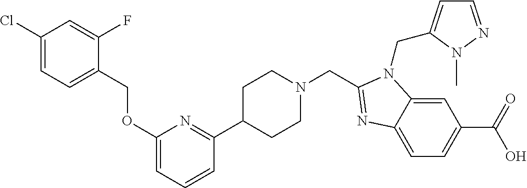 [SMILES: Cn1nccc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1- yl)methyl]-1-[(1-methyl- 1H-pyrazol-5-yl)methyl]-1H- benzimidazole-6-carboxylic acid |
| 297B | 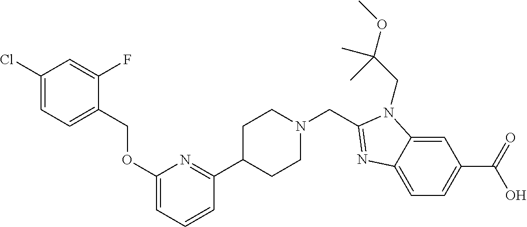 [SMILES: COC(C)(C)Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-(2-methoxy-2- methylpropyl)-1H- benzimidazole-6-carboxylic acid |
| 298B | 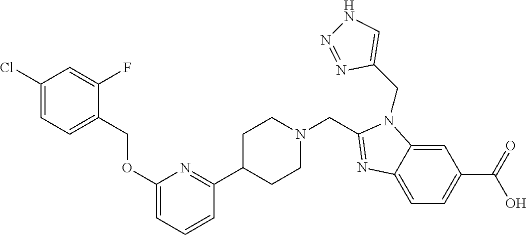 [SMILES: O=C(O)c1ccc2nc(CN3CCC(c4cccc(OCc5ccc(Cl)cc5F)n4)CC3)n(Cc3c[nH]nn3)c2c1] | 2-[(4-{6-[(4-chloro-2- fluorobenzyl)oxy]pyridin-2- yl}piperidin-1-yl)methyl]- 1-(1H-1,2,3-triazol-4-ylmethyl)- 1H-benzimidazole-6-carboxylic acid |
| 299B | 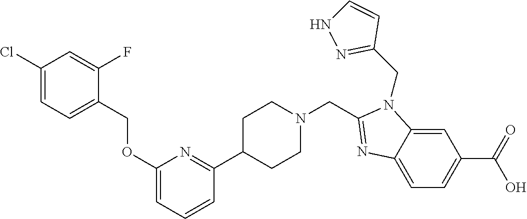 [SMILES: O=C(O)c1ccc2nc(CN3CCC(c4cccc(OCc5ccc(Cl)cc5F)n4)CC3)n(Cc3cc[nH]n3)c2c1] | 2-[(4-{6-[(4-chloro-2- fluorobenzyl)oxy]pyridin-2- yl}piperidin-1-yl)methyl]- 1-(1H-pyrazol-3-ylmethyl)- 1H-benzimidazole-6- carboxylic acid |
| 300B | 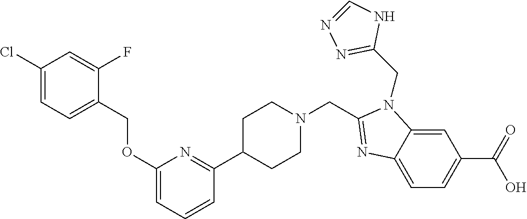 [SMILES: O=C(O)c1ccc2nc(CN3CCC(c4cccc(OCc5ccc(Cl)cc5F)n4)CC3)n(Cc3nnc[nH]3)c2c1] | 2-[(4-{6-[(4-chloro-2- fluorobenzyl)oxy]pyridin-2- yl}piperidin-1-yl)methyl]- 1-(4H-1,2,4-triazol-3-ylmethyl)- 1H-benzimidazole-6- carboxylic acid |
| 301B | 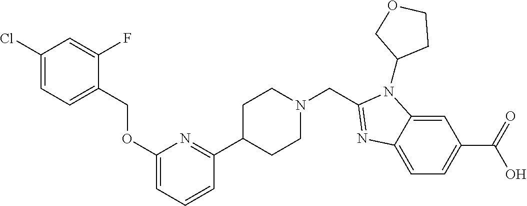 [SMILES: O=C(O)c1ccc2nc(CN3CCC(c4cccc(OCc5ccc(Cl)cc5F)n4)CC3)n(C3CCOC3)c2c1] | 2-[(4-{6-[(4-chloro-2- fluorobenzyl)oxy]pyridin-2- yl}piperidin-1-yl)methyl]-1- (tetrahydrofuran-3-yl)-1H- benzimidazole-6- carboxylic acid |
| 302B | 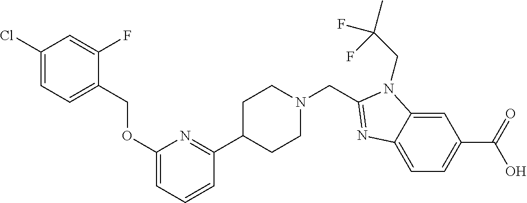 [SMILES: CC(F)(F)Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- (2,2-difluoropropyl)-1H- benzimidazole-6- carboxylic acid |
| 303B | 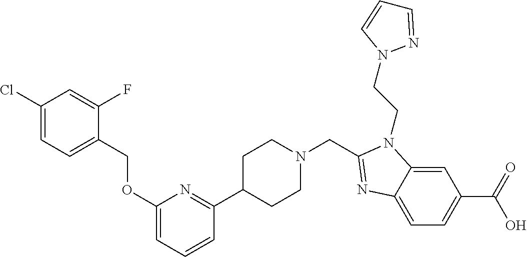 [SMILES: O=C(O)c1ccc2nc(CN3CCC(c4cccc(OCc5ccc(Cl)cc5F)n4)CC3)n(CCn3cccn3)c2c1] | 2-[(4-{6-[(4-chloro-2- fluorobenzyl)oxy]pyridin-2- yl}piperidin-1-yl)methyl]-1- [2-(1H-pyrazol-1-yl)ethyl]- 1H-benzimidazole-6- carboxylic acid |
| 304B | 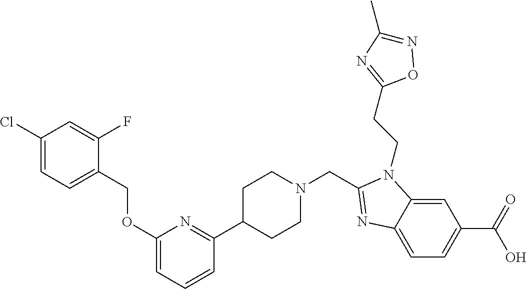 [SMILES: Cc1noc(CCn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin-2- yl}piperidin-1-yl)methyl]-1- [2-(3-methyl-1,2,4-oxadiazol- 5-yl)ethyl]-1H-benzimidazole- 6-carboxylic acid |
| 305B | 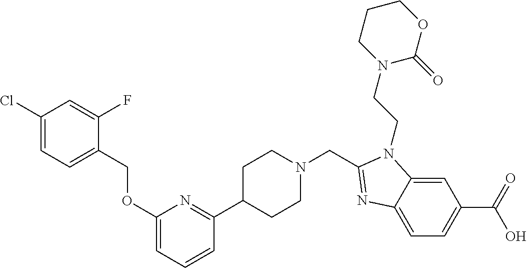 [SMILES: O=C(O)c1ccc2nc(CN3CCC(c4cccc(OCc5ccc(Cl)cc5F)n4)CC3)n(CCN3CCCOC3=O)c2c1] | 2-[(4-{6-[(4-chloro-2- fluorobenzyl)oxy]pyridin-2-yl} piperidin-1-yl)methyl]-1-[2-(2- oxo-1,3-oxazinan-3-yl)ethyl]- 1H-benzimidazole-6- carboxylic acid |
| 306B | 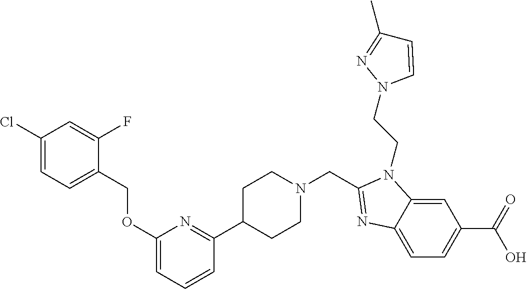 [SMILES: Cc1ccn(CCn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin-2-yl} piperidin-1-yl)methyl]-1-[2-(3- methyl-1H-pyrazol-1- yl)ethyl]-1H-benzimidazole- 6-carboxylic acid |
| 307B | 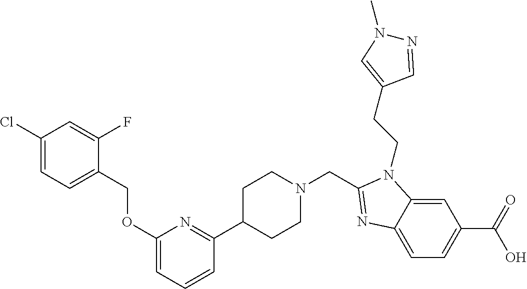 [SMILES: Cn1cc(CCn2c(CN3CCC(c4cccc(OCc5ccc(Cl)cc5F)n4)CC3)nc3ccc(C(=O)O)cc32)cn1] | 2-[(4-{6-[(4-chloro-2- fluorobenzyl)oxy]pyridin-2- yl}piperidin-1-yl)methyl]-1- [2-(1-methyl-1H-pyrazol-4- yl)ethyl]-1H-benzimidazole- 6-carboxylic acid |
| 308B | 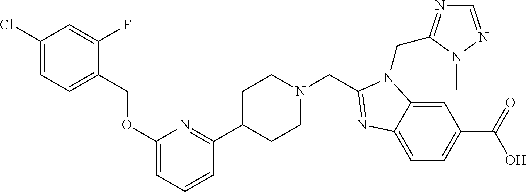 [SMILES: Cn1ncnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [(1-methyl-1H-1,2,4-triazol- 5-yl)methyl]-1H- benzimidazole-6-carboxylic acid |
| 309B | 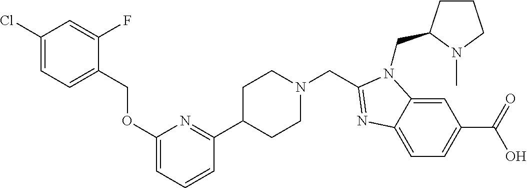 [SMILES: CN1CCC[C@@H]1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]- 1-{[(2R)-1-methylpyrrolidin- 2-yl]methyl}-1H- benzimidazole-6-carboxylic acid |
| 310B | 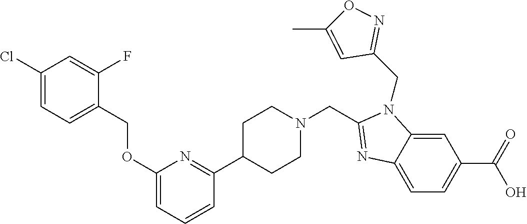 [SMILES: Cc1cc(Cn2c(CN3CCC(c4cccc(OCc5ccc(Cl)cc5F)n4)CC3)nc3ccc(C(=O)O)cc32)no1] | 2-[(4-{6-[(4-chloro-2- fluorobenzyl)oxy]pyridin-2- yl}piperidin-1-yl)methyl]-1- [(5-methyl-1,2-oxazol-3- yl)methyl]-1H- benzimidazole-6-carboxylic acid |
| 311B | 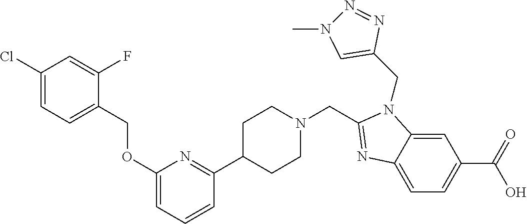 [SMILES: Cn1cc(Cn2c(CN3CCC(c4cccc(OCc5ccc(Cl)cc5F)n4)CC3)nc3ccc(C(=O)O)cc32)nn1] | 2-[(4-{6-[(4-chloro-2- fluorobenzyl)oxy]pyridin-2- yl}piperidin-1-yl)methyl]-1- [(1-methyl-1H-1,2,3-triazol-4- yl)methyl]-1H-benzimidazole- 6-carboxylic acid |
| 312B | 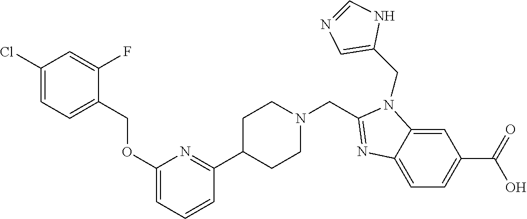 [SMILES: O=C(O)c1ccc2nc(CN3CCC(c4cccc(OCc5ccc(Cl)cc5F)n4)CC3)n(Cc3cnc[nH]3)c2c1] | 2-[(4-{6-[(4-chloro-2- fluorobenzyl)oxy]pyridin-2- yl}piperidin-1-yl)methyl]-1- (1H-imidazol-5-ylmethyl)- 1H-benzimidazole-6- carboxylic acid |
| 313B | 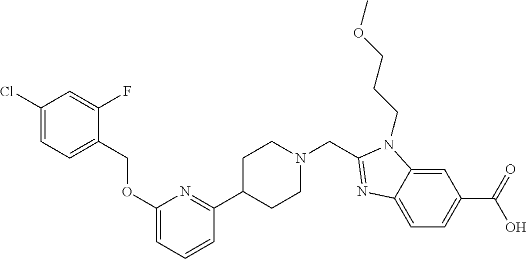 [SMILES: COCCCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin- 2-yl}piperidin-1-yl)methyl]- 1-(3-methoxypropyl)-1H- benzimidazole-6- carboxylic acid |
| 314B | 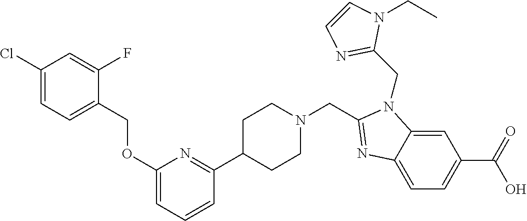 [SMILES: CCn1ccnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [(1-ethyl-1H-imidazol-2- yl)methyl]-1H- benzimidazole-6-carboxylic acid |
| 315B | 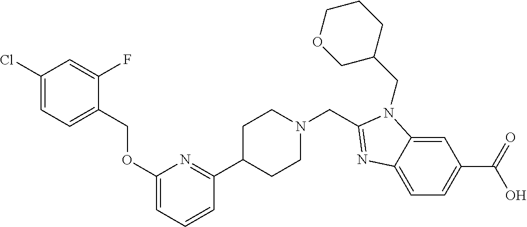 [SMILES: O=C(O)c1ccc2nc(CN3CCC(c4cccc(OCc5ccc(Cl)cc5F)n4)CC3)n(CC3CCCOC3)c2c1] | 2-[(4-{6-[(4-chloro-2- fluorobenzyl)oxy]pyridin-2-yl} piperidin-1-yl)methyl]-1- (tetrahydro-2H-pyran-3- ylmethyl)-1H-benzimidazole- 6-carboxylic acid |
| 316B | 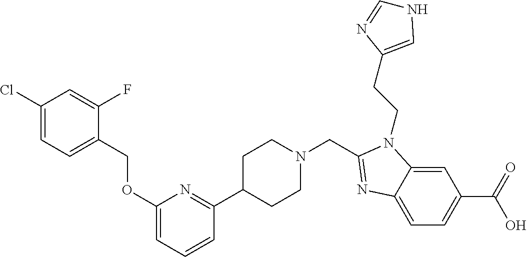 [SMILES: O=C(O)c1ccc2nc(CN3CCC(c4cccc(OCc5ccc(Cl)cc5F)n4)CC3)n(CCc3c[nH]cn3)c2c1] | 2-[(4-{6-[(4-chloro-2- fluorobenzyl)oxy]pyridin-2- yl}piperidin-1-yl)methyl]-1- [2-(1H-imidazol-4-yl)ethyl]- 1H-benzimidazole-6- carboxylic acid |
| 317B | 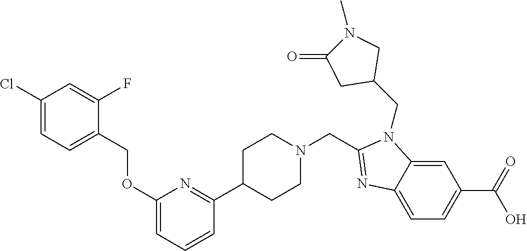 [SMILES: CN1CC(Cn2c(CN3CCC(c4cccc(OCc5ccc(Cl)cc5F)n4)CC3)nc3ccc(C(=O)O)cc32)CC1=O] | 2-[(4-{6-[(4-chloro-2- fluorobenzyl)oxy]pyridin-2- yl}piperidin-1-yl)methyl]-1- [(1-methyl-5-oxopyrrolidin- 3-yl)methyl]-1H- benzimidazole-6-carboxylic acid |
| 318B | 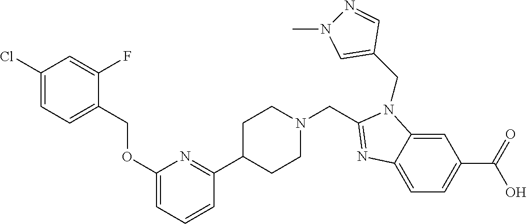 [SMILES: Cn1cc(Cn2c(CN3CCC(c4cccc(OCc5ccc(Cl)cc5F)n4)CC3)nc3ccc(C(=O)O)cc32)cn1] | 2-[(4-{6-[(4-chloro-2- fluorobenzyl)oxy]pyridin-2- yl}piperidin-1-yl)methyl]-1- [(1-methyl-1H-pyrazol-4- yl)methyl]-1H- benzimidazole-6-carboxylic acid |
| 319B | 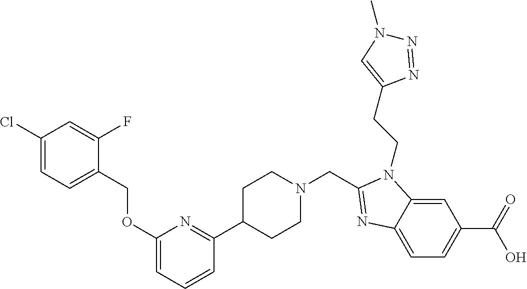 [SMILES: Cn1cc(CCn2c(CN3CCC(c4cccc(OCc5ccc(Cl)cc5F)n4)CC3)nc3ccc(C(=O)O)cc32)nn1] | 2-[(4-{6-[(4-chloro-2- fluorobenzyl)oxy]pyridin-2- yl}piperidin-1-yl)methyl]-1- [2-(1-methyl-1H-1,2,3-triazol- 4-yl)ethyl]-1H-benzimidazole- 6-carboxylicacid |
| 320B | 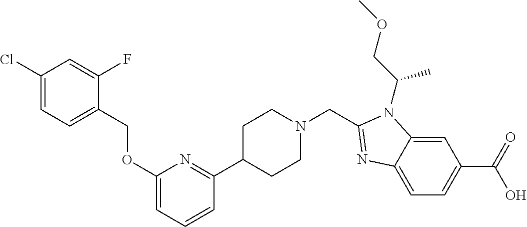 [SMILES: COC[C@H](C)n1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [(2S)-1-methoxypropan-2- yl]-1H-benzimidazole-6- carboxylic acid |
| 321B | 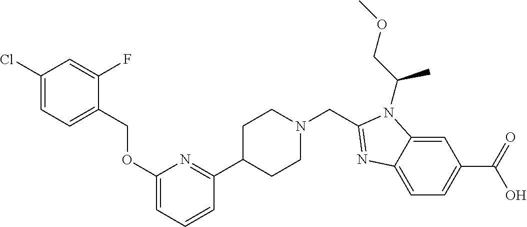 [SMILES: COC[C@@H](C)n1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [(2R)-1-methoxypropan-2- yl]-1H-benzimidazole-6- carboxylic acid |
| 322B | 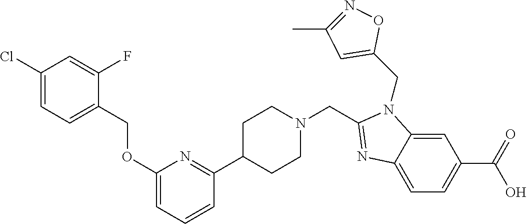 [SMILES: Cc1cc(Cn2c(CN3CCC(c4cccc(OCc5ccc(Cl)cc5F)n4)CC3)nc3ccc(C(=O)O)cc32)on1] | 2-[(4-{6-[(4-chloro-2- fluorobenzyl)oxy]pyridin- 2-yl}piperidin-1-yl)methyl]- 1-[(3-methyl-1,2-oxazol-5- yl)methyl]-1H- benzimidazole-6-carboxylic acid |
| 323B | 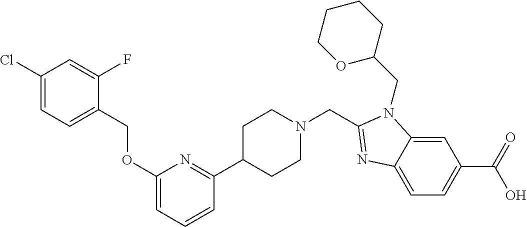 [SMILES: O=C(O)c1ccc2nc(CN3CCC(c4cccc(OCc5ccc(Cl)cc5F)n4)CC3)n(CC3CCCCO3)c2c1] | 2-[(4-{6-[(4-chloro-2- fluorobenzyl)oxy]pyridin-2- yl}piperidin-1-yl)methyl]- 1-(tetrahydro-2H-pyran-2- ylmethyl)-1H-benzimidazole- 6-carboxylic acid |
| 324B | 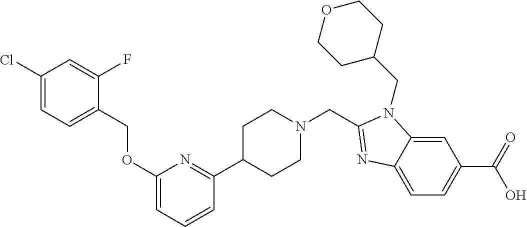 [SMILES: O=C(O)c1ccc2nc(CN3CCC(c4cccc(OCc5ccc(Cl)cc5F)n4)CC3)n(CC3CCOCC3)c2c1] | 2-[(4-{6-[(4-chloro-2- fluorobenzyl)oxy]pyridin-2- yl}piperidin-1-yl)methyl]- 1-(tetrahydro-2H-pyran-4- ylmethyl)-1H-benzimidazole- 6-carboxylic acid |
| 325B | 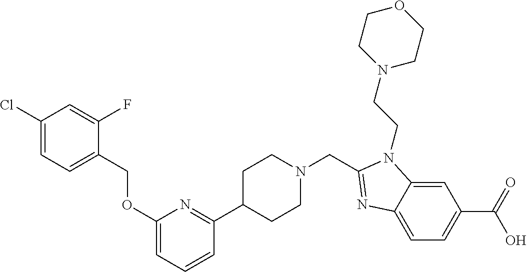 [SMILES: O=C(O)c1ccc2nc(CN3CCC(c4cccc(OCc5ccc(Cl)cc5F)n4)CC3)n(CCN3CCOCC3)c2c1] | 2-[(4-{6-[(4-chloro-2- fluorobenzyl)oxy]pyridin-2- yl}piperidin-1-yl)methyl]-1- [2-(morpholin-4-yl)ethyl]- 1H-benzimidazole-6- carboxylic acid |
| 326B | 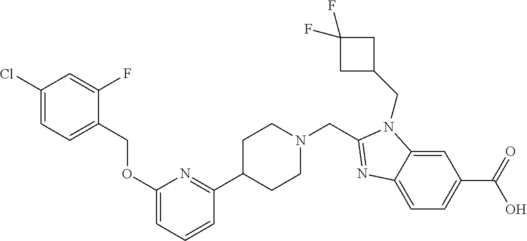 [SMILES: O=C(O)c1ccc2nc(CN3CCC(c4cccc(OCc5ccc(Cl)cc5F)n4)CC3)n(CC3CC(F)(F)C3)c2c1] | 2-[(4-{6-[(4-chloro-2- fluorobenzyl)oxy]pyridin-2- yl}piperidin-1-yl)methyl]-1- [(3,3-difluorocyclobutyl) methyl]-1H-benzimidazole- 6-carboxylic acid |
| 327B | 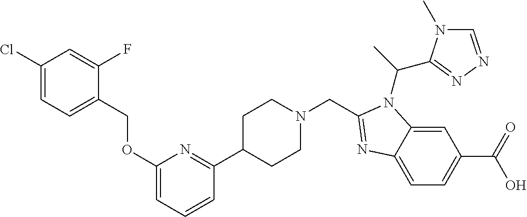 [SMILES: CC(c1nncn1C)n1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2-yl} piperidin-1-yl)methyl]-1-[1- (4-methyl-4H-1,2,4-triazol- 3-yl)ethyl]-1H-benzimidazole- 6-carboxylic acid |
| 328B | 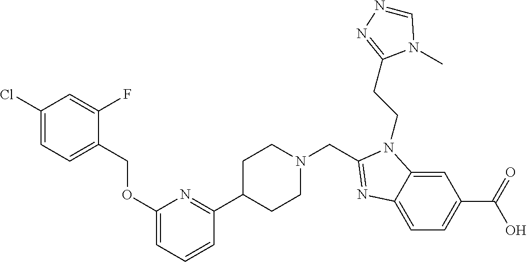 [SMILES: Cn1cnnc1CCn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2-yl} piperidin-1-yl)methyl]-1-[2-(4- methyl-4H-1,2,4-triazol-3-yl) ethyl]-1H-benzimidazole-6- carboxylic acid |
| 329B | 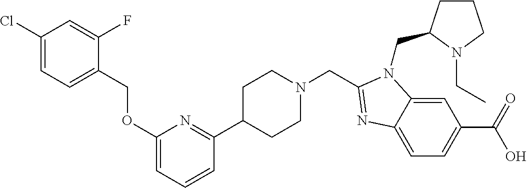 [SMILES: CCN1CCC[C@@H]1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- {[(2R)-1-ethylpyrrolidin-2- yl]methyl}-1H-benzimidazole- 6-carboxylic acid |
| 330B | 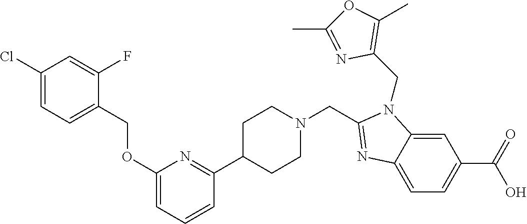 [SMILES: Cc1nc(Cn2c(CN3CCC(c4cccc(OCc5ccc(Cl)cc5F)n4)CC3)nc3ccc(C(=O)O)cc32)c(C)o1] | 2-[(4-{6-[(4-chloro-2- fluorobenzyl)oxy]pyridin-2- yl}piperidin-1-yl)methyl]-1- [(2,5-dimethyl-1,3-oxazol-4- yl)methyl]-1H-benzimidazole- 6-carboxylic acid |
| 331B | 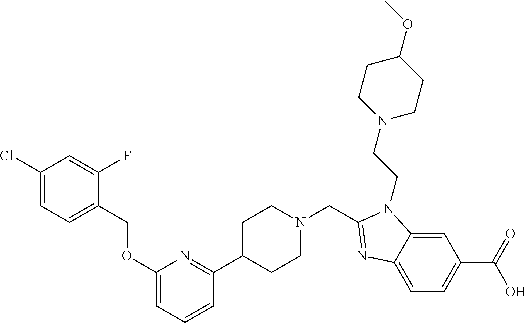 [SMILES: COC1CCN(CCn2c(CN3CCC(c4cccc(OCc5ccc(Cl)cc5F)n4)CC3)nc3ccc(C(=O)O)cc32)CC1] | 2-[(4-{6-[(4-chloro-2- fluorobenzyl)oxy]pyridin-2- yl}piperidin-1-yl)methyl]-1- [2-(4-methoxypiperidin-1- yl)ethyl]-1H-benzimidazole- 6-carboxylic acid |
| 332B | 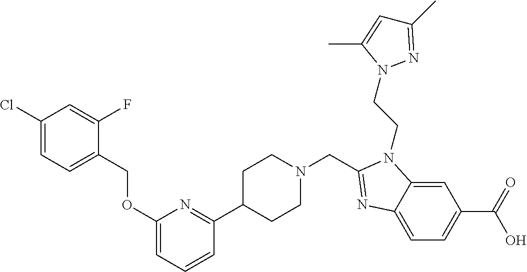 [SMILES: Cc1cc(C)n(CCn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin-2-yl} piperidin-1-yl)methyl]-1-[2- (3,5-dimethyl-1H-pyrazol-1- yl)ethyl]-1H-benzimidazole- 6-carboxylic acid |
| 333B | 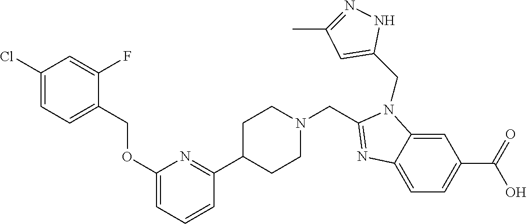 [SMILES: Cc1cc(Cn2c(CN3CCC(c4cccc(OCc5ccc(Cl)cc5F)n4)CC3)nc3ccc(C(=O)O)cc32)[nH]n1] | 2-[(4-{6-[(4-chloro-2- fluorobenzyl)oxy]pyridin-2- yl}piperidin-1-yl)methyl]-1- [(3-methyl-1H-pyrazol-5- yl)methyl]-1H- benzimidazole-6-carboxylic acid |
| 334B | 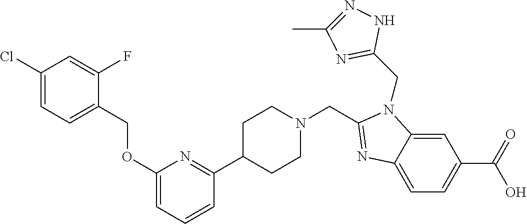 [SMILES: Cc1n[nH]c(Cn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin-2- yl}piperidin-1-yl)methyl]-1- [(3-methyl-1H-1,2,4-triazol- 5-yl)methyl]-1H- benzimidazole-6-carboxylic acid |
| 335B | 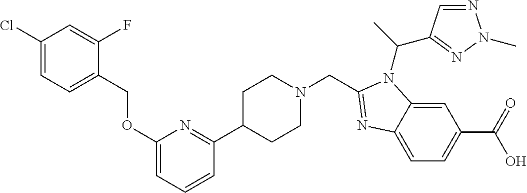 [SMILES: CC(c1cnn(C)n1)n1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]-1- [1-(2-methyl-2H-1,2,3-triazol- 4-yl)ethyl]-1H-benzimidazole- 6-carboxylic acid |
| 336B | 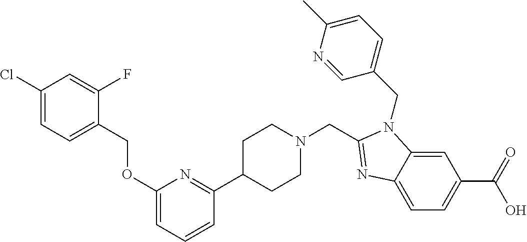 [SMILES: Cc1ccc(Cn2c(CN3CCC(c4cccc(OCc5ccc(Cl)cc5F)n4)CC3)nc3ccc(C(=O)O)cc32)cn1] | 2-[(4-{6-[(4-chloro-2- fluorobenzyl)oxy]pyridin-2- l} piperidin-1-yl)methyl]-1- [(6-methylpyridin-3-yl) methyl]-1H-benzimidazole- 6-carboxylic acid |
| 337B | 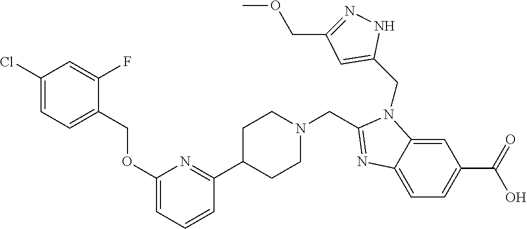 [SMILES: COCc1cc(Cn2c(CN3CCC(c4cccc(OCc5ccc(Cl)cc5F)n4)CC3)nc3ccc(C(=O)O)cc32)[nH]n1] | 2-[(4-{6-[(4-chloro-2- fluorobenzyl)oxy]pyridin-2- yl}piperidin-1-yl)methyl]- 1-{[3-(methoxymethyl)- 1H-pyrazol-5-yl]methyl}- 1H-benzimidazole-6- carboxylic acid |
| 338B | 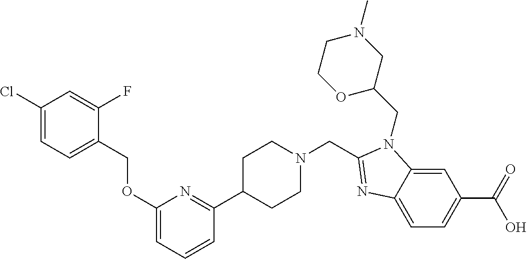 [SMILES: CN1CCOC(Cn2c(CN3CCC(c4cccc(OCc5ccc(Cl)cc5F)n4)CC3)nc3ccc(C(=O)O)cc32)C1] | 2-[(4-{6-[(4-chloro-2- fluorobenzyl)oxy]pyridin-2- yl}piperidin-1-yl)methyl]-1- [(4-methylmorpholin-2- yl)methyl]-1H- benzimidazole-6-carboxylic acid |
| 339B | 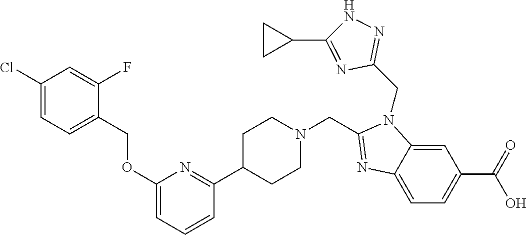 [SMILES: O=C(O)c1ccc2nc(CN3CCC(c4cccc(OCc5ccc(Cl)cc5F)n4)CC3)n(Cc3n[nH]c(C4CC4)n3)c2c1] | 2-[(4-{6-[(4-chloro-2- fluorobenzyl)oxy]pyridin-2- yl}piperidin-1-yl)methyl]-1- [(5-cyclopropyl-1H-1,2,4- triazol-3-yl)methyl]-1H- benzimidazole-6- carboxylic acid |
| 340B | 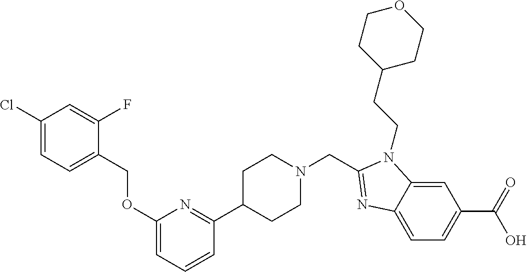 [SMILES: O=C(O)c1ccc2nc(CN3CCC(c4cccc(OCc5ccc(Cl)cc5F)n4)CC3)n(CCC3CCOCC3)c2c1] | 2-[(4-{6-[(4-chloro-2- fluorobenzyl)oxy]pyridin-2- yl}piperidin-1-yl)methyl]-1- [2-(tetrahydro-2H-pyran-4- yl)ethyl]-1H-benzimidazole- 6-carboxylic acid |
| 341B | 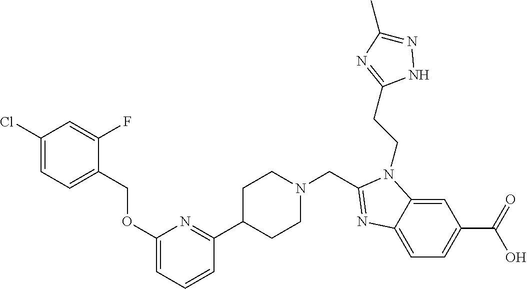 [SMILES: Cc1n[nH]c(CCn2c(CN3CCC(c4cccc(OCc5ccc(Cl)cc5F)n4)CC3)nc3ccc(C(=O)O)cc32)n1] | 2-[(4-{6-[(4-chloro-2- fluorobenzyl)oxy]pyridin-2- yl} piperidin-1-yl)methyl]-1- [2-(3-methyl-1H-1,2,4-triazol- 5-yl)ethyl]-1H-benzimidazole- 6-carboxylic acid |
| 342B | 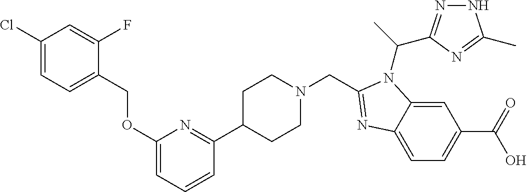 [SMILES: Cc1nc(C(C)n2c(CN3CCC(c4cccc(OCc5ccc(Cl)cc5F)n4)CC3)nc3ccc(C(=O)O)cc32)n[nH]1] | 2-[(4-{6-[(4-chloro-2- fluorobenzyl)oxy]pyridin-2- yl}piperidin-1-yl)methyl]-1- [1-(5-methyl-1H-1,2,4-triazol- 3-yl)ethyl]-1H-benzimidazole- 6-carboxylic acid |
| 343B | 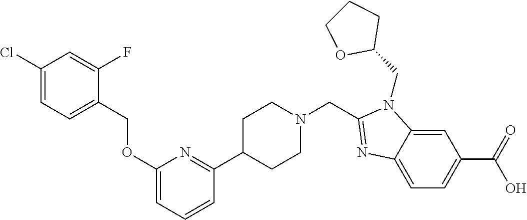 [SMILES: O=C(O)c1ccc2nc(CN3CCC(c4cccc(OCc5ccc(Cl)cc5F)n4)CC3)n(C[C@H]3CCCO3)c2c1] | 2-[(4-{6-[(4-chloro-2- fluorobenzyl)oxy]pyridin-2- yl}piperidin-1-yl)methyl]-1- [(2R)-tetrahydrofuran-2- ylmethyl]-1H-benzimidazole- 6-carboxylic acid |
| 344B | 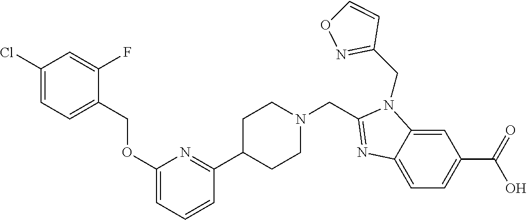 [SMILES: O=C(O)c1ccc2nc(CN3CCC(c4cccc(OCc5ccc(Cl)cc5F)n4)CC3)n(Cc3ccon3)c2c1] | 2-[(4-{6-[(4-chloro-2- fluorobenzyl)oxy]pyridin-2- yl}piperidin-1-yl)methyl]-1- (1,2-oxazol-3-ylmethyl)-1H- benzimidazole-6- carboxylic acid |
| 345B | 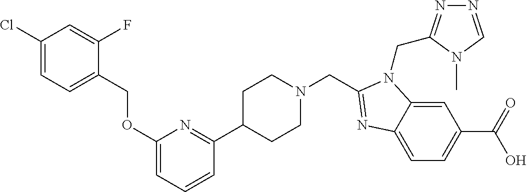 [SMILES: Cn1cnnc1Cn1c(CN2CCC(c3cccc(OCc4ccc(Cl)cc4F)n3)CC2)nc2ccc(C(=O)O)cc21] | 2-[(4-{6-[(4-chloro-2- fluorobenzyl)oxy]pyridin-2- yl}piperidin-1-yl)methyl]- 1-[(4-methyl-4H-1,2,4- triazol-3-yl)methyl]-1H- benzimidazole-6-carboxylic acid |
| 346B | 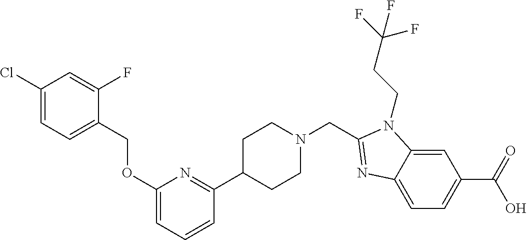 [SMILES: O=C(O)c1ccc2nc(CN3CCC(c4cccc(OCc5ccc(Cl)cc5F)n4)CC3)n(CCC(F)(F)F)c2c1] | 2-[(4-{6-[(4-chloro-2- fluorobenzyl)oxy]pyridin-2- yl}piperidin-1-yl)methyl]-1- (3,3,3-trifluoropropyl)-1H- benzimidazole-6- carboxylic acid |
| 347B | 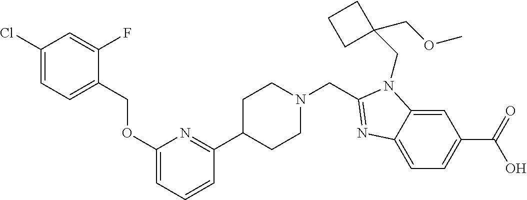 [SMILES: COCC1(Cn2c(CN3CCC(c4cccc(OCc5ccc(Cl)cc5F)n4)CC3)nc3ccc(C(=O)O)cc32)CCC1] | 2-[(4-{6-[(4-chloro-2- fluorobenzyl)oxy]pyridin-2- yl}piperidin-1-yl)methyl]- 1-{[1-(methoxymethyl) cyclobutyl]methyl}-1H- benzimidazole-6- carboxylic acid |
| 348B | 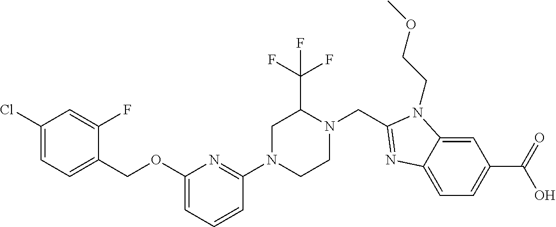 [SMILES: COCCn1c(CN2CCN(c3cccc(OCc4ccc(Cl)cc4F)n3)CC2C(F)(F)F)nc2ccc(C(=O)O)cc21] | 2-((4-(6-((4-Chloro-2- fluorobenzyl)oxy)pyridin-2- yl)-2-(trifluoromethyl) piperazin-1-yl)methyl)-1- (2-methoxyethyl)-1H- benzo[d]imidazole-6- carboxylic acid |
| 349B | 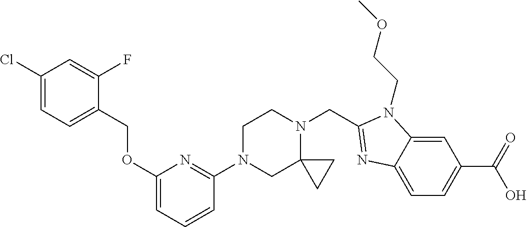 [SMILES: COCCn1c(CN2CCN(c3cccc(OCc4ccc(Cl)cc4F)n3)CC23CC3)nc2ccc(C(=O)O)cc21] | 2-[(7-{6-[(4-chloro-2- fluorobenzyl)oxy]pyridin-2- yl}-4,7-diazaspiro[2.5]oct- 4-yl)methyl]-1-(2- methoxyethyl)-1H- benzimidazole-6- carboxylic acid |
| 350B | 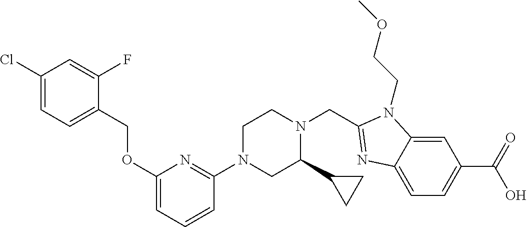 [SMILES: COCCn1c(CN2CCN(c3cccc(OCc4ccc(Cl)cc4F)n3)C[C@@H]2C2CC2)nc2ccc(C(=O)O)cc21] | 2-{[(2S)-4-{6-[(4-chloro-2- fluorobenzyl)oxy]pyridin-2- yl}-2-cyclopropylpiperazin- 1-yl]methyl}- 1-(2- methoxyethyl)-1H- benzimidazole-6- carboxylic acid |
| 351B | 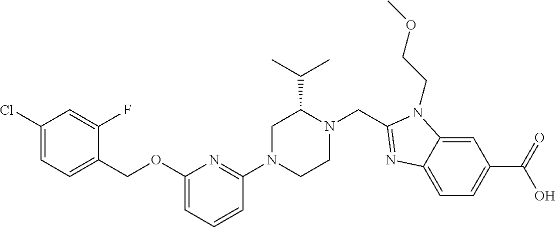 [SMILES: COCCn1c(CN2CCN(c3cccc(OCc4ccc(Cl)cc4F)n3)C[C@@H]2C(C)C)nc2ccc(C(=O)O)cc21] | 2-{[(2S)-4-{6-[(4-chloro-2- fluorobenzyl)oxy]pyridin-2- yl}-2-(propan-2- yl)piperazin-1-yl]methyl}- 1-(2-methoxyethyl)-1H- benzimidazole-6- carboxylic acid |
| 352B | 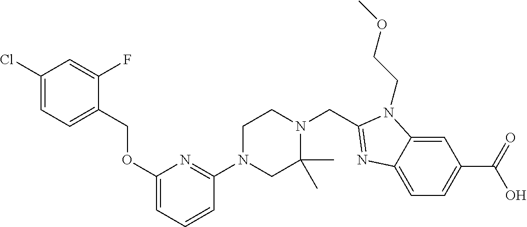 [SMILES: COCCn1c(CN2CCN(c3cccc(OCc4ccc(Cl)cc4F)n3)CC2(C)C)nc2ccc(C(=O)O)cc21] | 2-[(4-{6-[(4-chloro-2- fluorobenzyl)oxy]pyridin-2- yl}-2,2-dimethylpiperazin- 1-yl)methyl]-1-(2- methoxyethyl)-1H- benzimidazole-6- carboxylic acid |
| 353B | 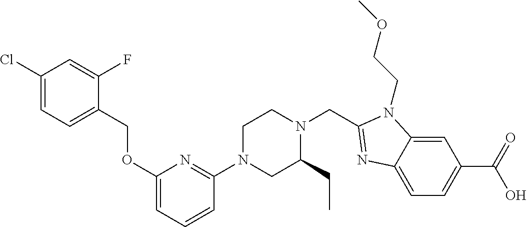 [SMILES: CC[C@H]1CN(c2cccc(OCc3ccc(Cl)cc3F)n2)CCN1Cc1nc2ccc(C(=O)O)cc2n1CCOC] | 2-{[(2S)-4-{6-[(4-chloro-2- fluorobenzyl)oxy]pyridin-2- yl}-2-ethylpiperazin-1- yl]methyl}-1-(2- methoxyethyl)-1H- benzimidazole-6- carboxylic acid |
| 354B | 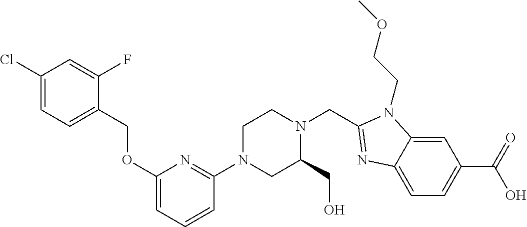 [SMILES: COCCn1c(CN2CCN(c3cccc(OCc4ccc(Cl)cc4F)n3)C[C@@H]2CO)nc2ccc(C(=O)O)cc21] | 2-{[(2R)-4-{6-[(4-chloro-2- fluorobenzyl)oxy]pyridin-2- yl}-2-(hydroxymethyl) piperazin-1-yl]methyl}-1- (2-methoxyethyl)-1H- benzimidazole-6- carboxylic acid |
| 355B | 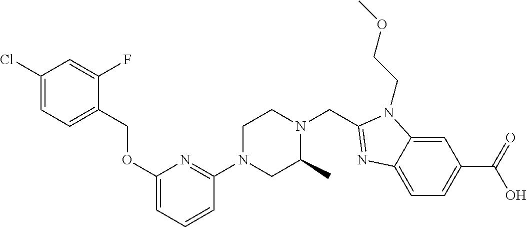 [SMILES: COCCn1c(CN2CCN(c3cccc(OCc4ccc(Cl)cc4F)n3)C[C@@H]2C)nc2ccc(C(=O)O)cc21] | 2-{[(2S)-4-{6-[(4-chloro-2- fluorobenzyl)oxy]pyridin-2- yl}-2-methylpiperazin-1- yl]methyl}-1-(2- methoxyethyl)-1H- benzimidazole-6- carboxylic acid |
| 356B | 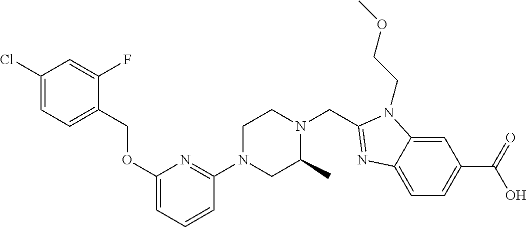 [SMILES: COCCn1c(CN2CCN(c3cccc(OCc4ccc(Cl)cc4F)n3)C[C@@H]2C)nc2ccc(C(=O)O)cc21] | 2-{[(2R)-4-{6-[(4-chloro-2- fluorobenzyl)oxy]pyridin-2- yl}-2-methylpiperazin-1- yl]methyl}-1-(2- methoxyethyl)-1H- benzimidazole-6- carboxylic acid |
| 357B | 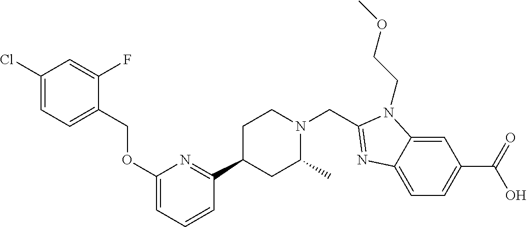 [SMILES: COCCn1c(CN2CC[C@H](c3cccc(OCc4ccc(Cl)cc4F)n3)C[C@H]2C)nc2ccc(C(=O)O)cc21] | trans 2-{[4-{6-[(4-Chloro-2- fluorobenzyl)oxy]pyridin-2- yl}-2-methylpiperidin-1- yl]methyl}-1-(2- methoxyethyl)-1H- benzimidazole-6- carboxylic acid |
| 358B | 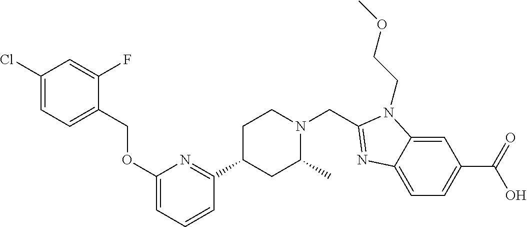 [SMILES: COCCn1c(CN2CC[C@@H](c3cccc(OCc4ccc(Cl)cc4F)n3)C[C@H]2C)nc2ccc(C(=O)O)cc21] | cis 2-{[4-{6-[(4-Chloro-2- fluorobenzyl)oxy]pyridin-2- yl}-2-methylpiperidin-1- yl]methyl}-1-(2- methoxyethyl)-1H- benzimidazole-6- carboxylic acid |
| 359B | 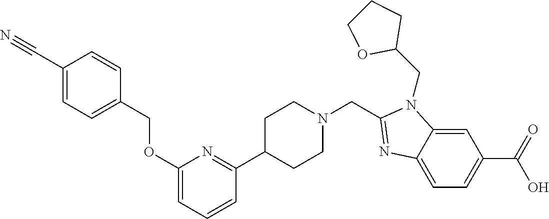 [SMILES: N#Cc1ccc(COc2cccc(C3CCN(Cc4nc5ccc(C(=O)O)cc5n4CC4CCCO4)CC3)n2)cc1] | 2-[(4-{6-[(4-cyanobenzyl) oxy]pyridin-2-yl}piperidin- 1-yl)methyl]-1- (tetrahydrofuran- 2-ylmethyl)-1H- benzimidazole-6-carboxylic acid |
| 360B | 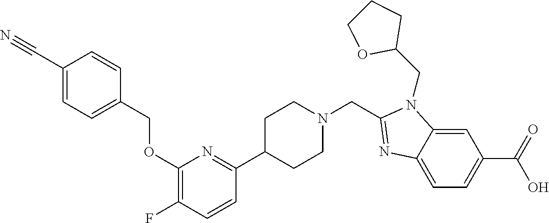 [SMILES: N#Cc1ccc(COc2nc(C3CCN(Cc4nc5ccc(C(=O)O)cc5n4CC4CCCO4)CC3)ccc2F)cc1] | 2-[(4-{6-[(4-cyanobenzyl) oxy]-5-fluoropyridin-2-yl} piperidin-1-yl)methyl]-1- (tetrahydrofuran-2-ylmethyl)- 1H-benzimidazole-6- carboxylic acid |
| 361B | 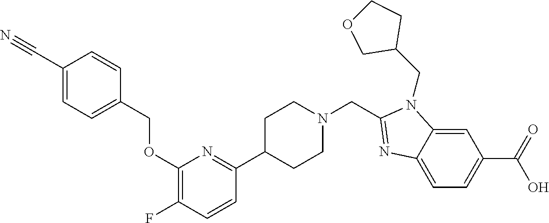 [SMILES: N#Cc1ccc(COc2nc(C3CCN(Cc4nc5ccc(C(=O)O)cc5n4CC4CCOC4)CC3)ccc2F)cc1] | 2-[(4-{6-[(4-cyanobenzyl) oxy]-5-fluoropyridin-2-yl} piperidin-1-yl)methyl]-1- (tetrahydrofuran-3-ylmethyl)- 1H-benzimidazole-6- carboxylic acid |
| 362B | 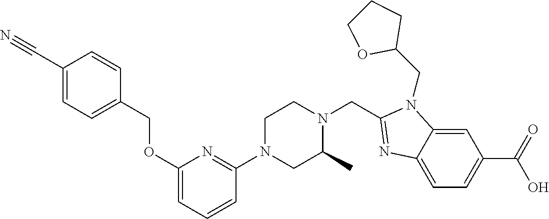 [SMILES: C[C@H]1CN(c2cccc(OCc3ccc(C#N)cc3)n2)CCN1Cc1nc2ccc(C(=O)O)cc2n1CC1CCCO1] | 2-{[(2S)-4-{6-[(4-cyanobenzyl) oxy]pyridin-2-yl}-2- methylpiperazin-1-yl]methyl}-1- (tetrahydrofuran-2-ylmethyl)-1H- benzimidazole-6-carboxylic acid |
| 363B | 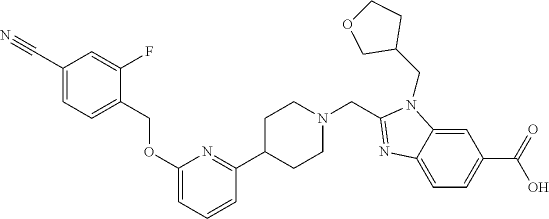 [SMILES: N#Cc1ccc(COc2cccc(C3CCN(Cc4nc5ccc(C(=O)O)cc5n4CC4CCOC4)CC3)n2)c(F)c1] | 2-[(4-{6-[(4-cyano-2- fluorobenzyl)oxy]pyridin- 2-yl}piperidin-1- yl)methyl]-1- (tetrahydrofuran-3- ylmethyl)-1H- benzimidazole-6- carboxylic acid |
| 364B | 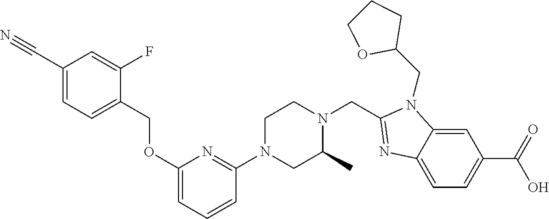 [SMILES: C[C@H]1CN(c2cccc(OCc3ccc(C#N)cc3F)n2)CCN1Cc1nc2ccc(C(=O)O)cc2n1CC1CCCO1] | 2-{[(2S)-4-{6-[(4-cyano-2- fluorobenzyl)oxy]pyridin-2- yl}-2-methylpiperazin-1- yl]methyl}-1- (tetrahydrofuran-2-ylmethyl)- 1H-benzimidazole-6- carboxylic acid |

In some embodiments, the GLP-1R agonist compound is a compound selected from the group consisting of:

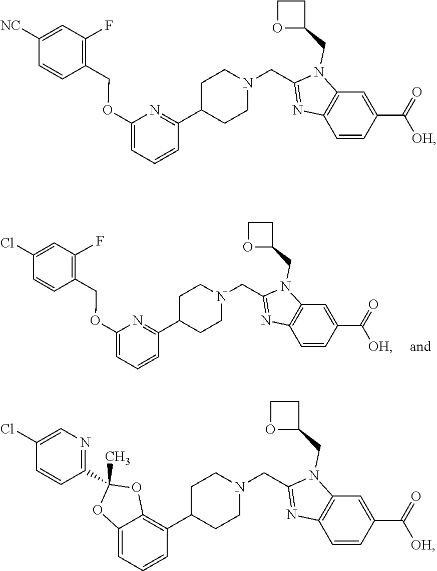 [SMILES: N#Cc1ccc(COc2cccc(C3CCN(Cc4nc5ccc(C(=O)O)cc5n4C[C@@H]4CCO4)CC3)n2)c(F)c1 ; O=C(O)c1ccc2nc(CN3CCC(c4cccc(OCc5ccc(Cl)cc5F)n4)CC3)n(C[C@@H]3CCO3)c2c1 ; C[C@]1(c2ccc(Cl)cn2)Oc2cccc(C3CCN(Cc4nc5ccc(C(=O)O)cc5n4C[C@@H]4CCO4)CC3)c2O1]

or a pharmaceutically acceptable salt thereof.

In some embodiments, the GLP-1R agonist compound is a compound of the structure:

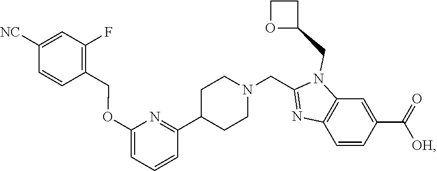 [SMILES: N#Cc1ccc(COc2cccc(C3CCN(Cc4nc5ccc(C(=O)O)cc5n4C[C@@H]4CCO4)CC3)n2)c(F)c1]

or a pharmaceutically acceptable salt thereof.

In some embodiments, the GLP-1R agonist compound is a compound of the structure:

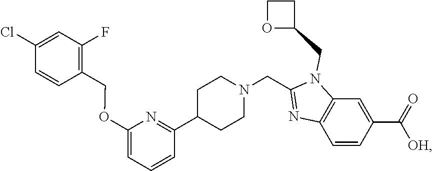 [SMILES: O=C(O)c1ccc2nc(CN3CCC(c4cccc(OCc5ccc(Cl)cc5F)n4)CC3)n(C[C@@H]3CCO3)c2c1]

or a pharmaceutically acceptable salt thereof.

In some embodiments, the GLP-1R agonist compound is a compound of the structure:

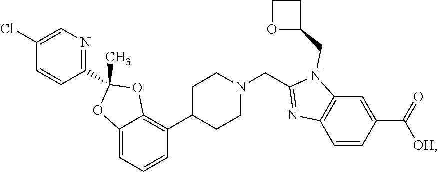 [SMILES: C[C@]1(c2ccc(Cl)cn2)Oc2cccc(C3CCN(Cc4nc5ccc(C(=O)O)cc5n4C[C@@H]4CCO4)CC3)c2O1]

or a pharmaceutically acceptable salt thereof.

### GLP-1R Modulating Compounds

In one embodiment, the GLP-1 modulating compound is a small molecule GLP-1R agonist. In one embodiment, the GLP-1 modulating compound is a small molecule GLP-1R glucagon dual receptor agonist.

The GIPR antagonist compounds of the disclosure can be administered to a subject in combination with one or more of the GLP-1R agonist compounds described herein.

In one embodiment, the GLP-1R agonist is a compound of Formula D-I:

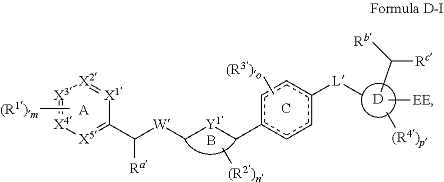

or a pharmaceutically acceptable salt thereof,
wherein:
   [symbol] indicates a single bond or a double bond;
   X^{1′}, X^{2′}, X^{3′}, X^{4′}, and X^{5′} are each independently selected from N and CH;
   W is selected from O, S, CR^{5′}R^{6′}, and NR^{5′a};
   ring B is 6-membered heteroaryl, 6-membered monocyclic heterocyclyl, or phenyl, wherein Y^{1′} is selected from N, NH, CH, and CH₂;
   ring C is cyclohexyl, phenyl, or pyridyl;
   L′ is CHR^{d′}, O, S, or NR^{5′a};
   ring D is bicyclic heteroaryl;
   EE is —COOH or a carboxylic group surrogate, optionally, the carboxylic group surrogate is:

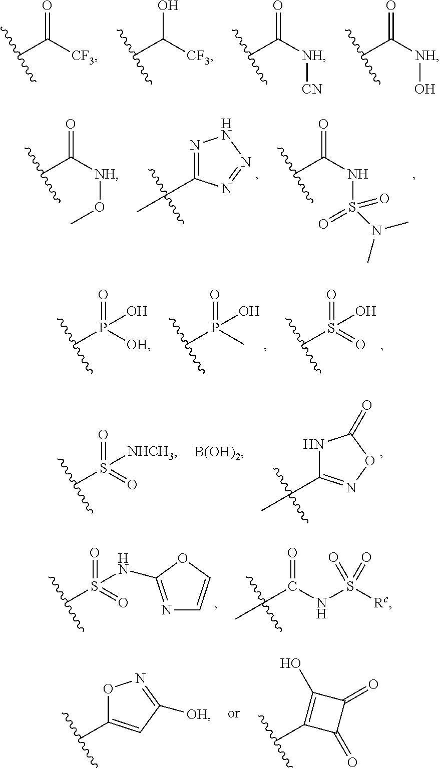

each R^{a′} and R^{b′} are independently selected from hydrogen, deuterium, halogen, —CN, C₁-C₆alkyl, C₁-C₆alkoxy, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆alkyl or C₁-C₆alkoxy represented by R^{a′}/R^{b′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{a′}/R^{b′} or in the group represented by R^{a′}/R^{b′} is optionally substituted with one or more groups selected from halogen, oxo, CN, OH, C₁-C₃ alkyl (optionally substituted with 1 to 3 groups selected from F, OH, and —OCH₃), and C₁-C₃ alkoxy (optionally substituted with 1 to 3 groups selected from F, OH, and —OCH₃), and NR^{5′a}R^{6′a};
each R^{c′} and R^{d′} are independently selected from hydrogen, deuterium, halogen, —CN, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{c′}/R^{d′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{c′}/R^{d′} or in the group represented by R^{c′}/R^{d′} is optionally substituted with one or more groups selected from halogen, oxo, CN, and NR^{5′a}R^{6′a};
each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{2′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{2′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{2′} or in the group represented by R^{2′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{3′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{3′} or in the group represented by R^{3′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆ alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃);
R^{5′} and R^{6′} are each independently selected from hydrogen, deuterium, halogen, CN, OH, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{5′} or R^{6′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{5′} or R^{6′} or in the group represented by R^{5′} or R^{6′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
R^{5′a} and R^{6′a} are each independently selected from hydrogen and C₁-C₆ alkyl;
wherein optionally two R^{1′}; two R^{2′}; two R^{3′}; two R^{4′}; R^{1′} and R^{2′}; R^{2′} and R^{3′}; R^{a′} and R^{1′}; R^{a′} and R^{2′}; R^{1′} and any of R^{5′}, R^{5′a} (in the group represented by W) or R^{6′}; R^{a′} and any of R^{5′}, R^{5′a} (in the group represented by W) or R^{6′}; R^{2′} and any of R^{5′}, R^{5′} (in the group represented by W) or R^{6′}; R^{5′} and R^{6′}; any of two groups selected from R^{c′}, R^{d′}, R^{e′}, and R^{f′}; or R^{4′} and any one of R^{c′}, R^{d′}, R^{e′}, and R^{f′}; taken together with their respective intervening carbon or hetero atom(s), form phenyl, 5-6 membered heteroaryl, 4-8 membered saturated or partially saturated cycloalkyl or 4-8 membered saturated or partially saturated heterocyclyl, and each of which is optionally substituted with one or more groups selected from halogen, —CN, —OH, CF₃, C₁-C₆ alkyl, C₁-C₆ alkoxy, —NH₂, —NHC₁-C₆ alkyl, —N(C₁-C₆ alkyl)₂, oxo, and saturated or partially saturated C₃-C₆ cycloalkyl, wherein the C₁-C₆ alkyl and C₁-C₆ alkoxy is optionally substituted with one or more groups selected from halogen, oxo, CN, CF₃, OH, OCH₃, OCH₂CH₃, and saturated or partially saturated C₃-C₆ cycloalkyl, wherein the cycloalkyl is optionally substituted with one or more groups selected from halogen, oxo, CN, CF₃, OH, OCH₃, OCH₂CH₃;
m′ is an integer selected from 0, 1, 2, 3, and 4;
n′ is an integer selected from 0, 1, 2, 3, 4, and 5;
o′ is an integer selected from 0, 1, 2, 3, and 4; and
p′ is an integer selected from 0, 1, 2, 3, and 4.

In one embodiment, the GLP-1R agonist is a compound of Formula D-II:

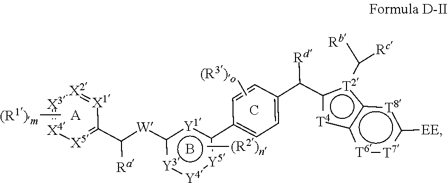

or a pharmaceutically acceptable salt thereof,
wherein:
   X^{1′}, X^{2′}, X^{3′}, X^{4′}, and X^{5′} are each independently selected from N and CH; wherein no more than three of X^{1′}, X^{2′}, X^{3′}, X^{4′}, and X^{5′} are N, and wherein ring A does not contain 3 nitrogen ring atoms at 3 contiguous positions;
   ring B is 6-membered heteroaryl or phenyl, wherein Y^{1′}, Y^{3′}, Y^{4′}, and Y^{5′} are each independently selected from N or CH; wherein there are no more than 3 nitrogen ring atoms in ring B, and wherein ring B does not contain 3 nitrogen ring atoms at 3 contiguous positions;
   T^{2′} is selected from N and C;
   T^{4′} is selected from N, NR^{4′}, O, S, and CR^{4′};
   T^{6′}, T^{7′}, and T^{8′} are each independently selected from N and CR^{4′};
      wherein no more than 4 of T^{2′}, T^{4′}, T^{6′}, T^{7′}, and T^{8′} are selected from N, O, and S.

In one embodiment, the GLP-1R agonist is a compound of Formula D-I or D-II, or a pharmaceutically acceptable salt thereof, wherein:
W is O, NH, or CH₂;
R^{a′} is H, CH₃, or CF₃;
R^{b′} is selected from hydrogen, C₁-C₆ alkyl, C₁-C₆ alkoxy, NR^{5′a}R^{6′a}, 6-10 membered aryl, 5-6 membered heteroaryl, 3-6 membered saturated or partially saturated cycloalkyl and 3-7 membered saturated or partially saturated heterocyclyl, wherein the C₁-C₆ alkyl or C₁-C₆ alkoxy represented by R^{b′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{b′} or in the group represented by R^{b′} is optionally substituted with one or more groups selected from halogen, oxo (when R^{b′} is non-aromatic), CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
R^{c′} is selected from hydrogen, halogen, and C₁-C₄ alkyl optionally substituted with one or more groups selected from halogen and hydroxy;
R^{d′} is H, F, CH₃, or CF₃; and
each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo (when R^{1′} is non-aromatic), CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{2′} and R^{3′} are independently selected from H, deuterium, halogen, —CN, OH, oxo, C₁-C₆ alkyl, C₁-C₆ alkoxy, NR^{5′a}R^{6′a}, wherein the C₁-C₆ alkyl or C₁-C₆ alkoxy represented by R^{2′} and/or R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃);
each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃); and
o′ is an integer selected from 0, 1, 2, 3, and 4.

In one embodiment, the GLP-1R agonist is a compound of Formula D-III:

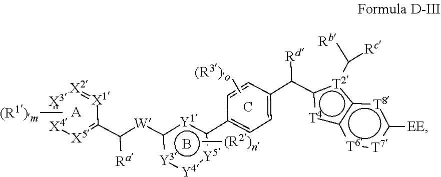

or a pharmaceutically acceptable salt, stereoisomer, solvate, or hydrate thereof, wherein R^{4′} is H, F, Cl, methyl, or methoxy.

In one embodiment, the GLP-1R agonist is a compound of Formula D-I, D-II, or D-III, or a pharmaceutically acceptable salt, stereoisomer, solvate, or hydrate thereof, wherein:

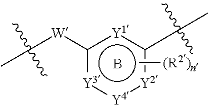

is

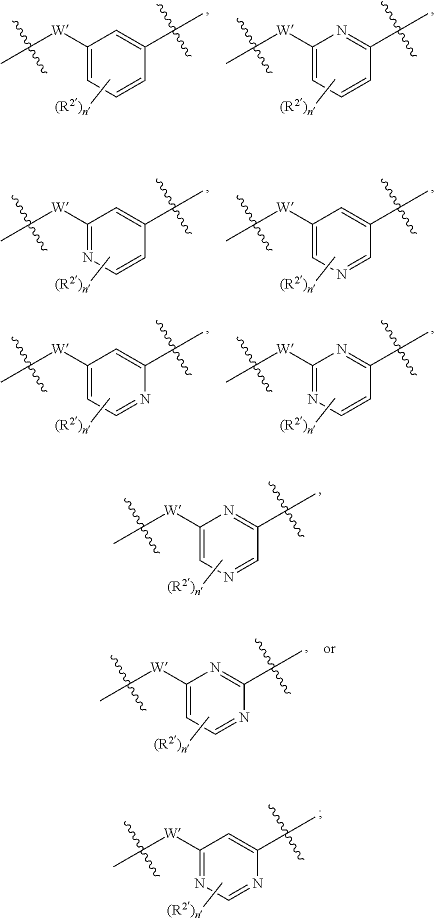

n′ is an integer selected from 0, 1, 2, 3, and 4.

In one embodiment, the GLP-1R agonist is a compound of Formula D-I, D-II, or D-III, or a pharmaceutically acceptable salt, stereoisomer, solvate, or hydrate thereof, wherein:
ring A is:

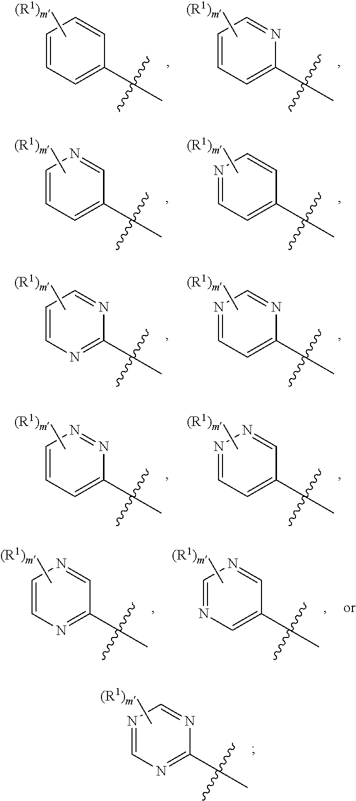

each R¹ is independently selected from halogen, OH, CN, C₁-C₄ alkyl, C₁-C₄ haloalkyl, C₁-C₄hydroxyalkyl, C₁-C₄alkoxy, C₁-C₄haloalkoxy, C₁-C₄hydroxyalkoxy, C₂-C₄ alkenyl, C₂-C₄ alkynyl, —NH₂, —NHC₁-C₄ alkyl, —N(C₁-C₄ alkyl)₂; and
m′ is an integer selected from 0, 1, and 2.

In one embodiment, the GLP-1R agonist is a compound of Formula D-I, D-II, or D-III, or a pharmaceutically acceptable salt, stereoisomer, solvate, or hydrate thereof, wherein EE is COOH.

In one embodiment, the GLP-1R agonist is a compound of Formula D-I, D-II, or D-III, or a pharmaceutically acceptable salt, stereoisomer, solvate, or hydrate thereof, wherein: R^{b′} is:

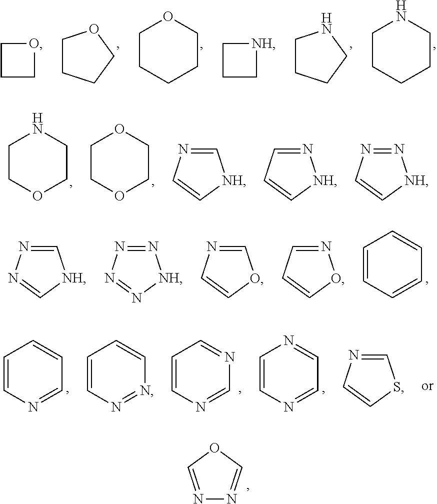

each of which is optionally substituted with 1 or 2 groups selected from halogen, oxo (when R^{b′} is non-aromatic), CN, NR^{5′a}R^{6′a}, C₁-C₄ alkyl, and C₁-C₄ alkoxy, wherein the C₁-C₄ alkyl or C₁-C₄alkoxy in the group represented by R^{b′} is optionally substituted with 1 or 2 groups selected from F, OH, and OCH₃.

In one embodiment, the GLP-1R agonist is a compound of Formula D-I, D-II, or D-III, or a pharmaceutically acceptable salt, stereoisomer, solvate, or hydrate thereof, wherein

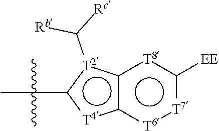

is:

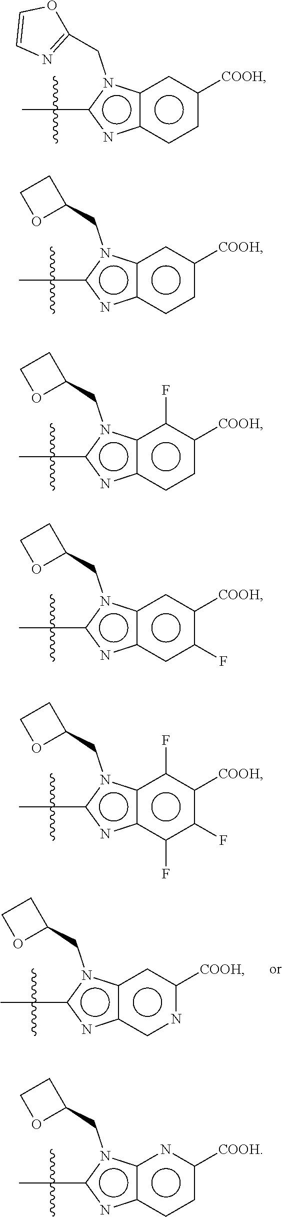

In one embodiment, the GLP-1R agonist is a compound of Formula D-I, D-II, or D-III, or a pharmaceutically acceptable salt, stereoisomer, solvate, or hydrate thereof, wherein:
R^{3′} is halogen, CN, OH, C₁-C₄ alkyl, C₁-C₄ haloalkyl, C₁-C₄alkoxy, or NR^{5′a}R^{6′a}; and
o′ is an integer selected from 0, 1, 2, 3, and 4.

In one embodiment, the GLP-1R agonist is a compound of Formula D-I, D-II, or D-III, or a pharmaceutically acceptable salt, stereoisomer, solvate, or hydrate thereof, wherein R^{2′} is independently selected from deuterium, halogen, —CN, OH, C₁-C₂ alkyl, C₁-C₂ haloalkyl, and C₁-C₂ alkoxy; and n is an integer selected from 0, 1, 2, 3, and 4. In one embodiment, the GLP-1R agonist is a compound of Formula D-I, D-II, or D-III, or a pharmaceutically acceptable salt, stereoisomer, solvate, or hydrate thereof, wherein:
ring A is

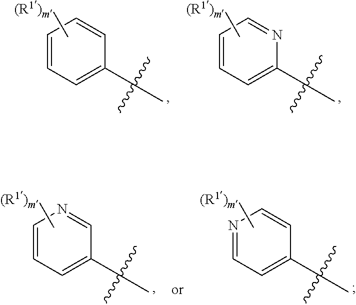

each R^{1′} is independently selected from halogen, OH, CN, C₁-C₂ alkyl, C₁-C₂ haloalkyl, C₁-C₂ hydroxyalkyl, C₁-C₂alkoxy, C₁-C₂haloalkoxy, C₁-C₂hydroxyalkoxy, and C₂-C₄ alkynyl; and
m′ is an integer selected from 0, 1, and 2.

In one embodiment, the GLP-1R agonist is a compound of Formula D-I, D-II, or D-III, or a pharmaceutically acceptable salt, stereoisomer, solvate, or hydrate thereof, wherein

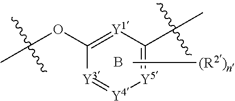

is

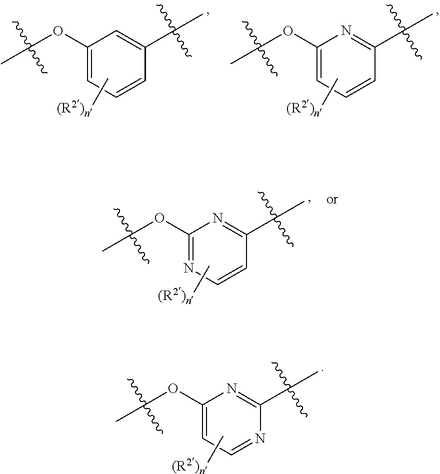

In one embodiment, the GLP-1R agonist is a compound of Formula D-I, D-II, or D-III, or a pharmaceutically acceptable salt, stereoisomer, solvate, or hydrate thereof, wherein

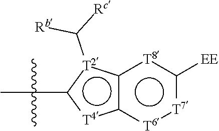

is

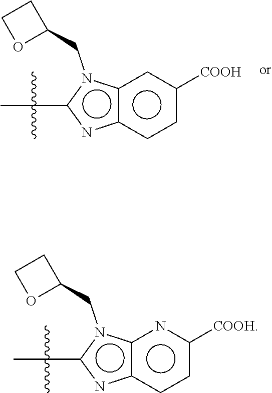

In one embodiment, the GLP-1R agonist is a compound of Formula D-I, D-II, or D-III, or a pharmaceutically acceptable salt, stereoisomer, solvate, or hydrate thereof, wherein each R^{2′} is independently selected from halogen or deuterium; and n is an integer selected from 0, 1, and 2, provided that when R^{2′} is deuterium, ring B is fully substituted with deuterium. In one embodiment, the GLP-1R agonist is a compound of Formula D-I, D-II, or D-III, or a pharmaceutically acceptable salt, stereoisomer, solvate, or hydrate thereof, wherein R^{3′} is F, Cl, or CH₃; and o′ is 0, 1, or 2.

In one embodiment, the GLP-1R agonist is a compound of Formula D-I, D-II, or D-III, or a pharmaceutically acceptable salt, stereoisomer, solvate, or hydrate thereof, wherein ring A is:

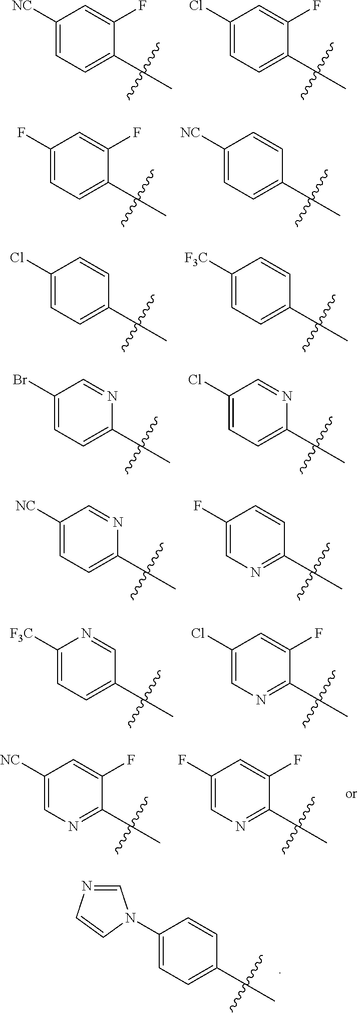

In one embodiment, the GLP-1R agonist is a compound of Formula D-I, D-II, or D-II, or a pharmaceutically acceptable salt, stereoisomer, solvate, or hydrate thereof, wherein ring A is

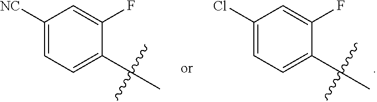 [SMILES: *c1ccc(C#N)cc1F ; *c1ccc(Cl)cc1F]

In one embodiment, the GLP-1R agonist is a compound of Formula D-I, D-II, or D-III, or a pharmaceutically acceptable salt, stereoisomer, solvate, or hydrate thereof, wherein ring B is

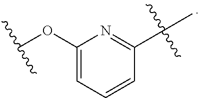 [SMILES: *Oc1cccc(*)n1]

In one embodiment, the GLP-1R agonist is a compound of Formula D-I1 or D-I2:

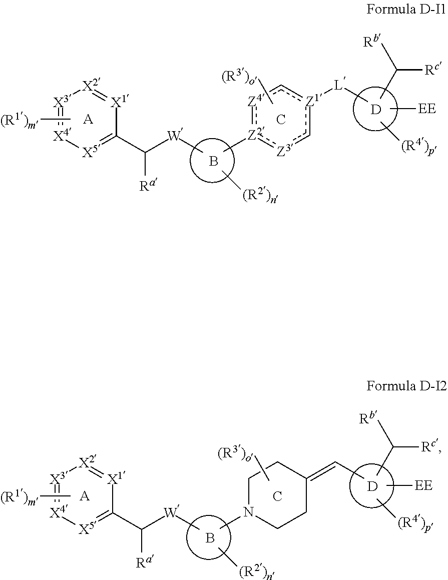

or a pharmaceutically acceptable salt, stereoisomer, solvate, or hydrate thereof, wherein:
[symbol] indicates a single bond or a double bond;
X^{1′}, X^{2′}, X^{3′}, X^{4′}, and X^{5′} are each independently selected from N and CH; wherein no more than three of X^{1′}, X^{2′}, X^{3′}, X^{4′}, and X^{5′} are N, and wherein ring A does not contain 3 nitrogen ring atoms at 3 contiguous positions;
W is selected from O, S, CR^{5′}R^{6′}, and NR5′^{a};
ring B is 6-membered heteroaryl, 6-membered monocyclic heterocyclyl, or phenyl;
Z^{1′} and Z^{2′} are each independently selected from N, C, and CH;
Z^{3′} and Z^{4′} are each independently selected from a bond, CH, CH₂, CH═CH, CH₂CH₂, CH₂CH, and CHCH₂, wherein ring C contains no more than two double bonds;
L′ is CHR^{d′}, O, S, or NR^{5′a};
ring D is bicyclic heteroaryl;
EE is —COOH or a carboxylic group surrogate, optionally, the carboxylic group surrogate is:

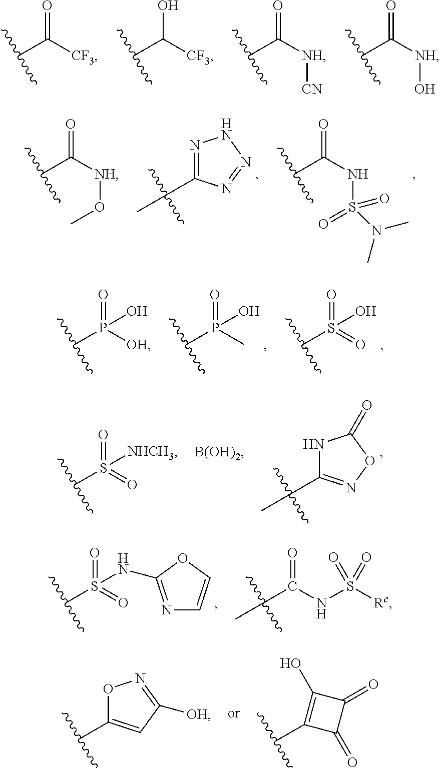

each R^{a′} and R^{b′} are independently selected from hydrogen, deuterium, halogen, —CN, C₁-C₆alkyl, C₁-C₆alkoxy, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆alkyl or C₁-C₆alkoxy represented by R^{a′}/R^{b′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{a′}/R^{b′} or in the group represented by R^{a′}/R^{b′} is optionally substituted with one or more groups selected from halogen, oxo, CN, OH, C₁-C₃ alkyl (optionally substituted with 1 to 3 groups selected from F, OH, and —OCH₃), and C₁-C₃ alkoxy (optionally substituted with 1 to 3 groups selected from F, OH, and —OCH₃), and NR^{5′a}R^{6′a};
each R^{c′} and R^{d′} are independently selected from hydrogen, deuterium, halogen, —CN, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{c′}/R^{d′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{c′}/R^{d′} or in the group represented by R^{c′}/R^{d′} is optionally substituted with one or more groups selected from halogen, oxo, CN, and NR^{5′a}R^{6′a};
each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{2′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{2′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{2′} or in the group represented by R^{2′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{3′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{3′} or in the group represented by R^{3′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆ alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃);
R^{5′} and R^{6′} are each independently selected from hydrogen, deuterium, halogen, CN, OH, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{5′} or R^{6′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{5′} or R^{6′} or in the group represented by R^{5′} or R^{6′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
R^{5′a} and R^{6′a} are each independently selected from hydrogen and C₁-C₆ alkyl;
wherein optionally two R^{1′}; two R^{2′}; two R^{3′}; two R^{4′}; R^{1′} and R^{2′}; R^{2′} and R^{3′}; R^{a′} and R^{1′}; R^{a′} and R^{2′}; R^{1′} and any of R^{5′}, R^{5′a} (in the group represented by W) or R^{6′}; R^{a′} and any of R^{5′}, R^{5′a} (in the group represented by W) or R^{6′}; R^{2′} and any of R^{5′}, R^{5′a} (in the group represented by W) or R^{6′}; R^{5′} and R^{6′}; any of two groups selected from R^{c′}, R^{d′}, R^{e′}, and R^{f′}; or R^{4′} and any one of R^{c′}, R^{d′}, R^{e′}, and R^{f′}; taken together with their respective intervening carbon or hetero atom(s), form phenyl, 5-6 membered heteroaryl, 4-8 membered saturated or partially saturated cycloalkyl or 4-8 membered saturated or partially saturated heterocyclyl, or the carbon atom of —C(R^{a′})—, W′, and R², taken together with two adjacent carbon atoms of ring B form

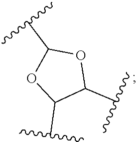 [SMILES: *C1OC(*)C(*)O1]

and each of which is optionally substituted with one or more groups selected from halogen, —CN, —OH, CF₃, C₁-C₆ alkyl, C₁-C₆ alkoxy, —NH₂, —NHC₁-C₆ alkyl, —N(C₁-C₆ alkyl)₂, oxo, and saturated or partially saturated C₃-C₆ cycloalkyl, wherein the C₁-C₆ alkyl and C₁-C₆ alkoxy is optionally substituted with one or more groups selected from halogen, oxo, CN, CF₃, OH, OCH₃, OCH₂CH₃, and saturated or partially saturated C₃-C₆ cycloalkyl, wherein the cycloalkyl is optionally substituted with one or more groups selected from halogen, oxo, CN, CF₃, OH, OCH₃, OCH₂CH₃;
m′ is an integer selected from 0, 1, 2, 3, and 4;
n′ is an integer selected from 0, 1, 2, 3, 4, and 5;
o′ is an integer selected from 0, 1, 2, 3, and 4; and
p′ is an integer selected from 0, 1, 2, 3, and 4.

In one embodiment, the GLP-1R agonist is a compound of Formula D-IA, D-IB, D-IC, or D-ID:

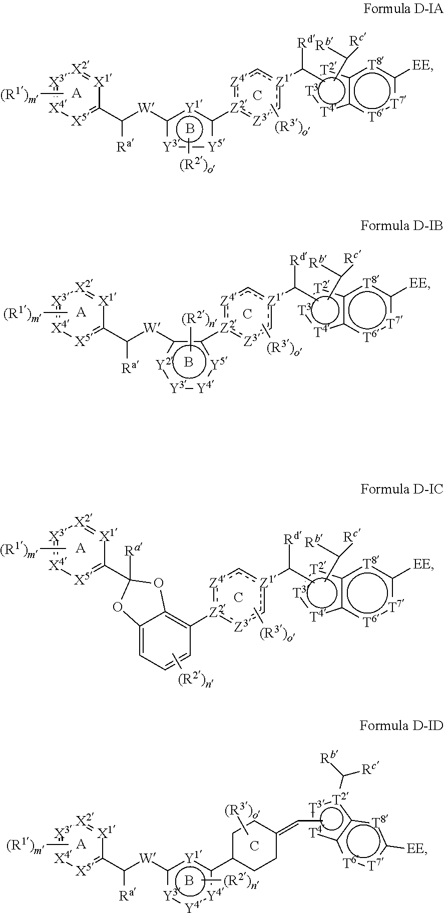

or a pharmaceutically acceptable salt, stereoisomer, solvate, or hydrate thereof, wherein:
[symbol] indicates a single bond or a double bond;
X^{1′}, X^{2′}, X^{3′}, X^{4′}, and X^{5′} are each independently selected from N and CH; wherein no more than three of X^{1′}, X^{2′}, X^{3′}, X^{4′}, and X^{5′} are N, and wherein ring A does not contain 3 nitrogen ring atoms at 3 contiguous positions;
W is selected from O, S, CR^{5′}R^{6′}, and NR5′^{a};
Y^{1′} is N or CH;
Y^{3′} and Y^{5′} are each independently selected from N, CH, O, and S;
Y^{4′} is absent, N, or CH;
Y^{2′a} are each independently n or CH;
Y^{3′a} and Y^{5′a} are each independently selected from N, CH, O, and S; and
Y^{4′a} is absent, N, or CH;
wherein there are no more than 3 hetero ring atoms in ring B, and wherein ring B does not contain 3 hetero ring atoms at 3 contiguous positions;
Z^{1′} and Z^{2′} are each independently selected from N, C, and CH; wherein at least one of Z^{1′} and Z^{2′} is N;
Z^{3′} and Z^{4′} are each independently selected from a bond, CH, CH₂, CH═CH, CH₂CH₂, CH₂CH, and CHCH₂, wherein ring C contains no more than two double bonds;
T², T³, and T⁴ are each independently selected from N, NR⁴, O, S, C, and CR^{4′};
T^{6′}, T^{7′}, and T^{8′} are each independently selected from N and CR^{4′};
wherein no more than 4 of T^{2′}, T^{3′}, T^{4′}, T^{6′}, T^{7′}, and T^{8′} are selected from N, O, and S;
and the remainder of the variables are as defined in in Formula D-I1 or D-I2.

In one embodiment, the GLP-1R agonist is a compound of Formula D-IIA, D-IIB, D-IIB′, D-IIC, or D-IID:

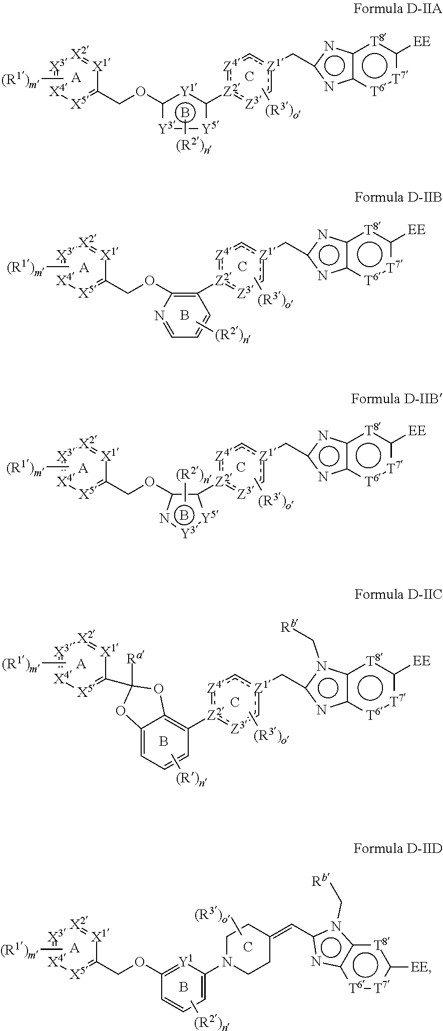

or a pharmaceutically acceptable salt, stereoisomer, solvate, or hydrate thereof, wherein the variables are as defined in the previous embodiments for the compounds represented by Formulas DI-1, DI-2, D-IA, D-IB, D-IC, or D-ID.

In some embodiments, the GLP-1R agonist is a compound of Formula D-I1, D-I2, D-IA, D-IB, B-IC, B-IIA, B-IIB, B-IIB′, B-IIC, or B-IID, or a pharmaceutically acceptable salt, stereoisomer, solvate, or hydrate thereof, wherein ring A is:

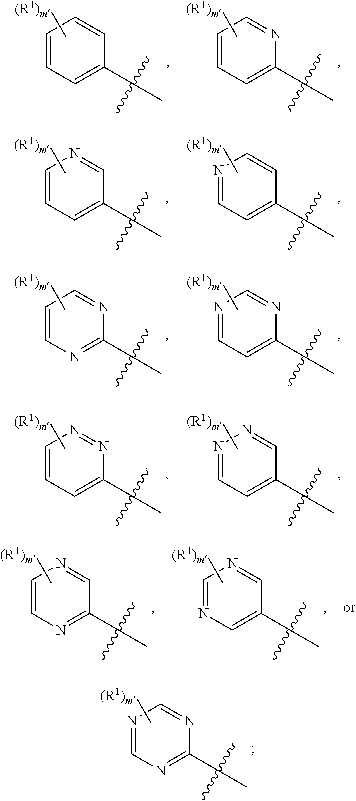

each R^{1′} is independently selected from halogen, OH, CN, C₁-C₄ alkyl, C₁-C₄ haloalkyl, C₁-C₄ hydroxyalkyl, C₁-C₄alkoxy, C₁-C₄haloalkoxy, C₁-C₄hydroxyalkoxy, C₂-C₄ alkenyl, C₂-C₄ alkynyl, —NH₂, —NHC₁-C₄ alkyl, —N(C₁-C₄ alkyl)₂; and m′ is an integer selected from 0, 1, and 2. In some embodiments, the GLP-1R agonist is a compound of Formula D-I1, D-I2, D-IA, D-IB, D-IC, D-IIA, D-IIB, D-IIB′, D-IIC, or D-IID, or a pharmaceutically acceptable salt, stereoisomer, solvate, or hydrate thereof, wherein ring A is:

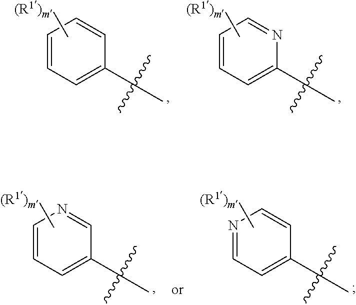

wherein each R^{1′} is independently selected from halogen, OH, CN, C₁-C₂ alkyl, C₁-C₂ haloalkyl, C₁-C₂ hydroxyalkyl, C₁-C₂alkoxy, C₁-C₂ haloalkoxy, C₁-C₂hydroxyalkoxy, C₂-C₄ alkenyl, C₂-C₄ alkynyl; and m′ is an integer selected from 0, 1, and 2. In one embodiment, ring A is

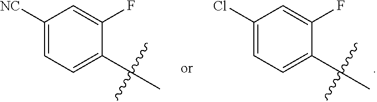 [SMILES: *c1ccc(C#N)cc1F ; *c1ccc(Cl)cc1F]

In one embodiment, the GLP-1R agonist is a compound of Formula D-I1, D-I2, D-IA, D-IB, D-IC, D-IIA, D-IIB, D-IIB′, D-IIC, or D-IID, or a pharmaceutically acceptable salt, stereoisomer, solvate, or hydrate thereof,
wherein

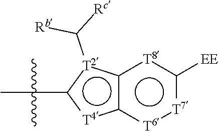

is:

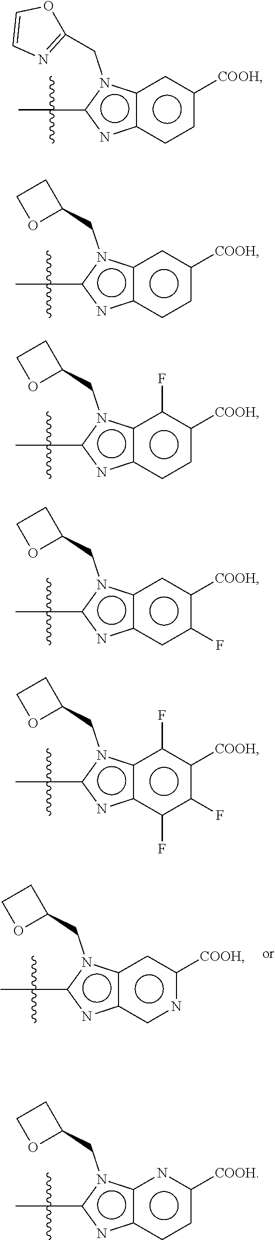

In one embodiment,

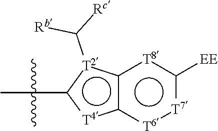

is

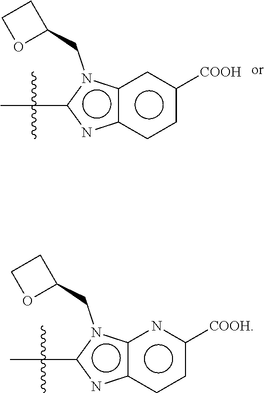

In one embodiment, the GLP-1R agonist is a compound of Formula D-I1, D-I2, D-IA, D-IB, D-IC, D-IIA, D-IIB, D-IIB′, D-IIC, or D-IID, or a pharmaceutically acceptable salt, stereoisomer, solvate, or hydrate thereof,
wherein

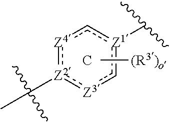

is:

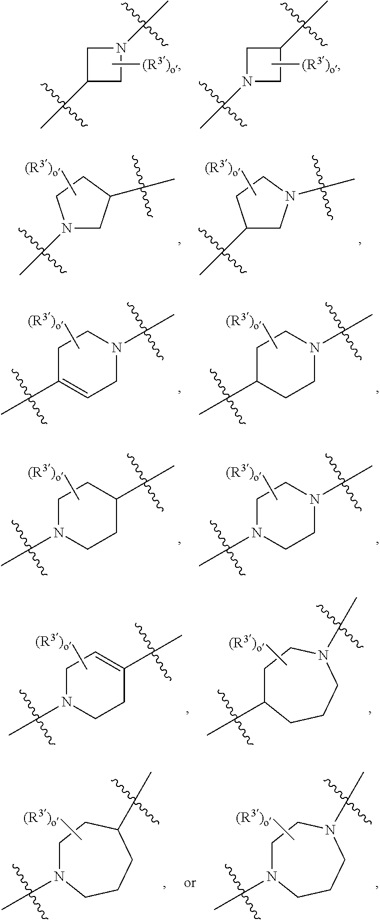

wherein R^{3′} is halogen, ON, OH, oxo, C₁-C₄ alkyl, C₁-C₄ haloalkyl, C₁-C₄ alkoxy, or NR^{5′a}NR^{6′a}; and
o′ is an integer selected from 0, 1, 2, and 3. In one embodiment,

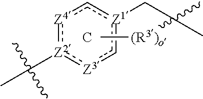

is

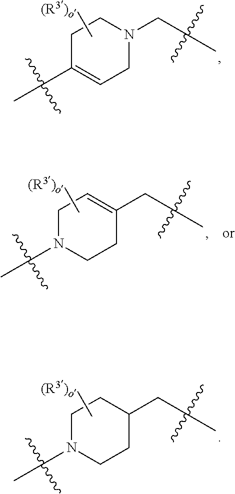

In one embodiment,

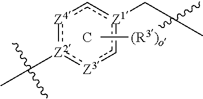

is

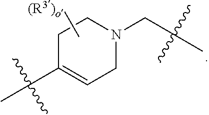

In one embodiment, the GLP-1R agonist is a compound of Formula D-IIA or D-IIB′, or a pharmaceutically acceptable salt, stereoisomer, solvate, or hydrate thereof, wherein

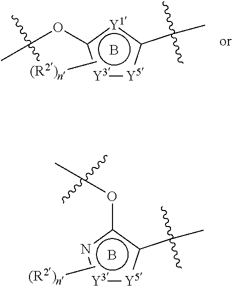

is

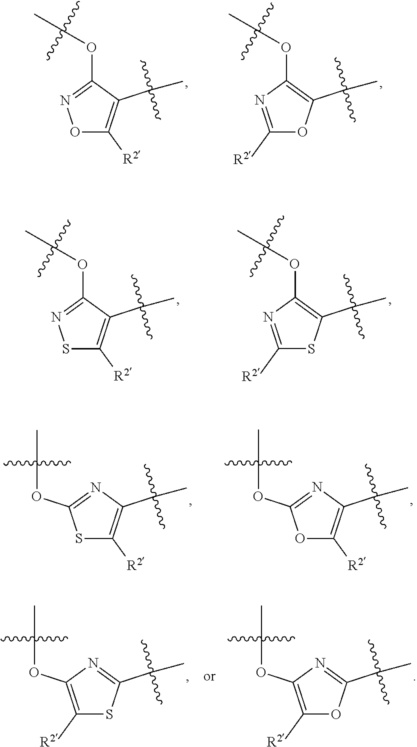

In one embodiment,

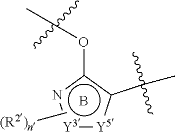

is

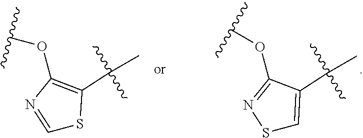 [SMILES: *Oc1ncsc1* ; *Oc1nscc1*]

In one embodiment,

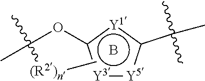

is

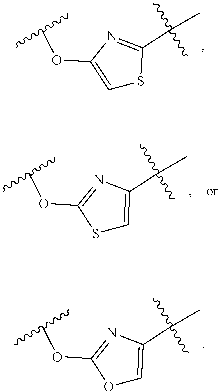 [SMILES: *Oc1csc(*)n1 ; *Oc1nc(*)cs1 ; *Oc1nc(*)co1]

In one embodiment, the GLP-1R agonist is a compound of Formula D-IID, or a pharmaceutically acceptable salt, stereoisomer, solvate, or hydrate thereof, wherein

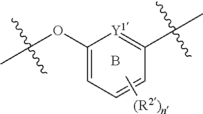

is

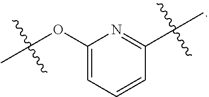 [SMILES: *Oc1cccc(*)n1]

The GLP-1R agonist compounds of the disclosure are compounds comprising a molecular weight of from about 400 Da to about 1000 Da, from about 400 Da to about 500 Da, from about 500 Da to about 600 Da, from about 600 Da to about 1000 Da, from about 450 Da to about 550 Da, from about 400 Da to about 450 Da, from about 450 Da to about 500 Da, from about 500 Da to about 550 Da, from about 550 Da to about 600 Da, from about 600 Da to about 650 Da, from about 650 Da to about 700 Da, from about 700 Da to about 750 Da, from about 750 Da to about 800 Da, from about 800 Da to about 850 Da, from about 850 Da to about 900 Da, from about 900 Da to about 950 Da, and from about 950 Da to about 1000 Da. In some embodiments, the GLP-1R agonist compounds of the disclosure comprise a molecular weight of from about 450 Da to about 500 Da. In some embodiments, the GLP-1R agonist compounds of the disclosure comprise a molecular weight of from about 500 Da to about 550 Da. In some embodiments, the GLP-1R agonist compounds of the disclosure comprise a molecular weight of from about 550 Da to about 600 Da. In some embodiments, the GLP-1R agonist compounds of the disclosure comprise a molecular weight of from about 600 Da to about 1000 Da.

TABLE 4 describes GLP-1R agonist compounds that can be used in combination with the GIPR antagonist compounds described herein.

TABLE 4
GLP-1R Agonist compounds for use in GIPR antagonist combination therapy

| GLP | | |
|---|---|---|
| 1R | | |
| Ag | Structure | IUPAC |
| 1C | 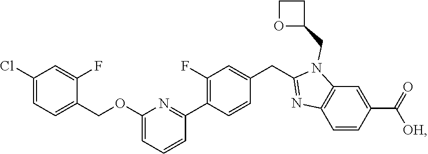 [SMILES: O=C(O)c1ccc2nc(Cc3ccc(-c4cccc(OCc5ccc(Cl)cc5F)n4)c(F)c3)n(C[C@@H]3CCO3)c2c1] | (S)-2-(4-(6-((4-chloro-2- fluorobenzyl)oxy)pyridin-2-yl)-3- fluorobenzyl)-1-(oxetan-2-ylmethyl)-1H- benzo[d]imidazole-6-carboxylic acid |
| 2C | 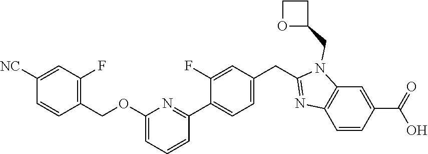 [SMILES: N#Cc1ccc(COc2cccc(-c3ccc(Cc4nc5ccc(C(=O)O)cc5n4C[C@@H]4CCO4)cc3F)n2)c(F)c1] | (S)-2-(4-(6-(4-cyano-2- fluorobenzyloxy)pyridin-2-yl)-3- fluorobenzyl)-1-(oxetan-2-ylmethyl)-1H- benzo[d]imidazole-6-carboxylic acid |
| 3C | 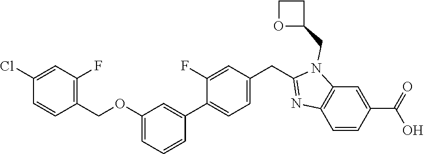 [SMILES: O=C(O)c1ccc2nc(Cc3ccc(-c4cccc(OCc5ccc(Cl)cc5F)c4)c(F)c3)n(C[C@@H]3CCO3)c2c1] | (S)-2-((3′-((4-chloro-2-fluorobenzyl)oxy)- 2-fluoro-[1,1′-biphenyl]-4-yl)methyl)-1- (oxetan-2-ylmethyl)-1H- benzo[d]imidazole-6-carboxylic acid |
| 4C | 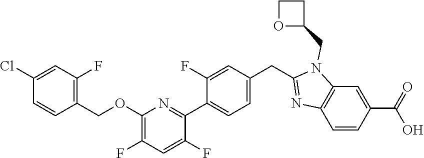 [SMILES: O=C(O)c1ccc2nc(Cc3ccc(-c4nc(OCc5ccc(Cl)cc5F)c(F)cc4F)c(F)c3)n(C[C@@H]3CCO3)c2c1] | (S)-2-(4-(6-((4-chloro-2- fluorobenzyl)oxy)-3,5-difluoropyridin-2- yl)-3-fluorobenzyl)-1-(oxetan-2- ylmethyl)-1H-benzo[d]imidazole-6- carboxylic acid |
| 5C | 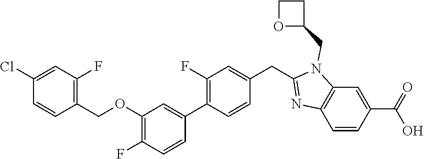 [SMILES: O=C(O)c1ccc2nc(Cc3ccc(-c4ccc(F)c(OCc5ccc(Cl)cc5F)c4)c(F)c3)n(C[C@@H]3CCO3)c2c1] | (S)-2-((3′-((4-chloro-2-fluorobenzyl)oxy)- 2,4′-difluoro-[1,1′-biphenyl]-4-yl)methyl)- 1-(oxetan-2-ylmethyl)-1H- benzo[d]imidazole-6-carboxylic acid |
| 6C | 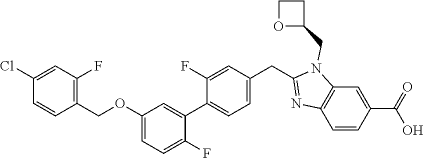 [SMILES: O=C(O)c1ccc2nc(Cc3ccc(-c4cc(OCc5ccc(Cl)cc5F)ccc4F)c(F)c3)n(C[C@@H]3CCO3)c2c1] | (S)-2-((5′-((4-chloro-2-fluorobenzyl)oxy)- 2,2′-difluoro-[1,1′-biphenyl]-4-yl)methyl)- 1-(oxetan-2-ylmethyl)-1H- benzo[d]imidazole-6-carboxylic acid |
| 7C | 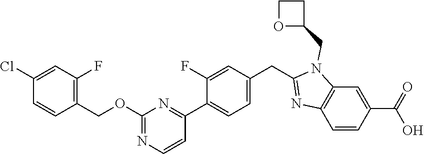 [SMILES: O=C(O)c1ccc2nc(Cc3ccc(-c4ccnc(OCc5ccc(Cl)cc5F)n4)c(F)c3)n(C[C@@H]3CCO3)c2c1] | (S)-2-(4-(2-((4-chloro-2- fluorobenzyl)oxy)pyrimidin-4-yl)-3- fluorobenzyl)-1-(oxetan-2-ylmethyl)-1H- benzo[d]imidazole-6-carboxylic acid |
| 8C | 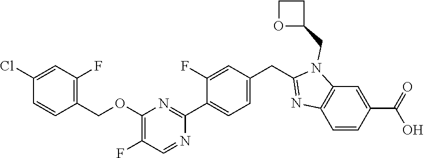 [SMILES: O=C(O)c1ccc2nc(Cc3ccc(-c4ncc(F)c(OCc5ccc(Cl)cc5F)n4)c(F)c3)n(C[C@@H]3CCO3)c2c1] | (S)-2-(4-(4-(4-chloro-2-fluorobenzyloxy)- 5-fluoropyrimidin-2-yl)-3-fluorobenzyl)-1- (oxetan-2-ylmethyl)-1H- benzo[d]imidazole-6-carboxylic acid |
| 9C | 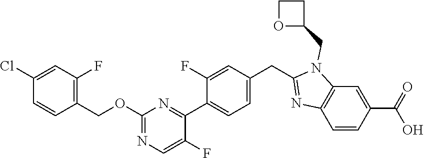 [SMILES: O=C(O)c1ccc2nc(Cc3ccc(-c4nc(OCc5ccc(Cl)cc5F)ncc4F)c(F)c3)n(C[C@@H]3CCO3)c2c1] | (S)-2-(4-(2-(4-chloro-2-fluorobenzyloxy)- 5-fluoropyrimidin-4-yl)-3-fluorobenzyl)-1- (oxetan-2-ylmethyl)-1H- benzo[d]imidazole-6-carboxylic acid |
| 10C | 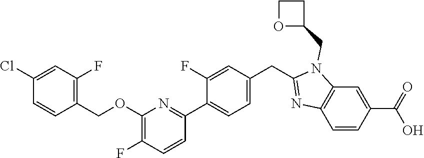 [SMILES: O=C(O)c1ccc2nc(Cc3ccc(-c4ccc(F)c(OCc5ccc(Cl)cc5F)n4)c(F)c3)n(C[C@@H]3CCO3)c2c1] | (S)-2-(4-(6-((4-chloro-2- fluorobenzyl)oxy)-5-fluoropyridin-2-yl)-3- fluorobenzyl)-1-(oxetan-2-ylmethyl)-1H- benzo[d]imidazole-6-carboxylic acid |
| 11C | 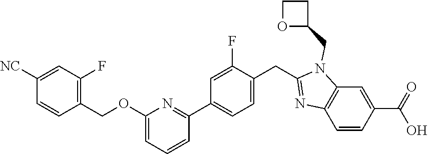 [SMILES: N#Cc1ccc(COc2cccc(-c3ccc(Cc4nc5ccc(C(=O)O)cc5n4C[C@@H]4CCO4)c(F)c3)n2)c(F)c1] | (S)-2-(4-(6-((4-cyano-2- fluorobenzyl)oxy)pyridin-2-yl)-2- fluorobenzyl)-1-(oxetan-2-ylmethyl)-1H- benzo[d]imidazole-6-carboxylic acid |
| 12C | 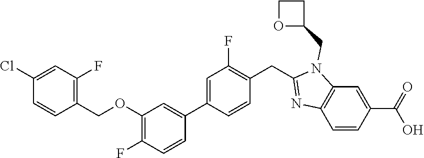 [SMILES: O=C(O)c1ccc2nc(Cc3ccc(-c4ccc(F)c(OCc5ccc(Cl)cc5F)c4)cc3F)n(C[C@@H]3CCO3)c2c1] | (S)-2-((3′-((4-chloro-2-fluorobenzyl)oxy)- 3,4′-difluoro-[1,1′-biphenyl]-4-yl)methyl)- 1-(oxetan-2-ylmethyl)-1H- benzo[d]imidazole-6-carboxylic acid |
| 13C | 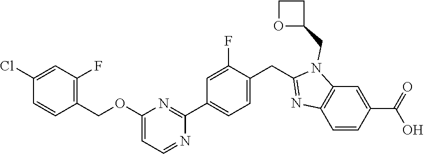 [SMILES: O=C(O)c1ccc2nc(Cc3ccc(-c4nccc(OCc5ccc(Cl)cc5F)n4)cc3F)n(C[C@@H]3CCO3)c2c1] | (S)-2-(4-(4-((4-chloro-2- fluorobenzyl)oxy)pyrimidin-2-yl)-2- fluorobenzyl)-1-(oxetan-2-ylmethyl)-1H- benzo[d]imidazole-6-carboxylic acid |
| 14C | 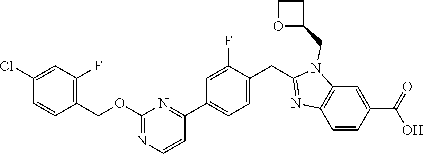 [SMILES: O=C(O)c1ccc2nc(Cc3ccc(-c4ccnc(OCc5ccc(Cl)cc5F)n4)cc3F)n(C[C@@H]3CCO3)c2c1] | (S)-2-(4-(2-((4-chloro-2- fluorobenzyl)oxy)pyrimidin-4-yl)-2- fluorobenzyl)-1-(oxetan-2-ylmethyl)-1H- benzo[d]imidazole-6-carboxylic acid |
| 15C | 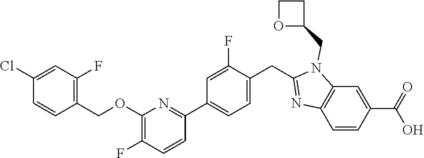 [SMILES: O=C(O)c1ccc2nc(Cc3ccc(-c4ccc(F)c(OCc5ccc(Cl)cc5F)n4)cc3F)n(C[C@@H]3CCO3)c2c1] | (S)-2-(4-(6-(4-chloro-2-fluorobenzyloxy)- 5-fluoropyridin-2-yl)-2-fluorobenzyl)-1- (oxetan-2-ylmethyl)-1H- benzo[d]imidazole-6-carboxylic acid |
| 16C | 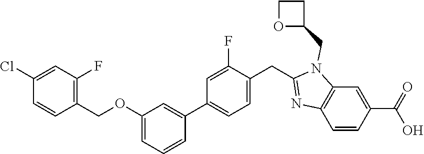 [SMILES: O=C(O)c1ccc2nc(Cc3ccc(-c4cccc(OCc5ccc(Cl)cc5F)c4)cc3F)n(C[C@@H]3CCO3)c2c1] | (S)-2-((3′-((4-chloro-2-fluorobenzyl)oxy)- 3-fluoro-[1, 1′-biphenyl]-4-yl)methyl)-1- (oxetan-2-ylmethyl)-1H- benzo[d]imidazole-6-carboxylic acid |
| 17C | 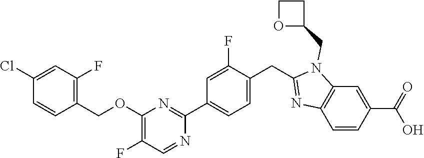 [SMILES: O=C(O)c1ccc2nc(Cc3ccc(-c4ncc(F)c(OCc5ccc(Cl)cc5F)n4)cc3F)n(C[C@@H]3CCO3)c2c1] | (S)-2-(4-(4-((4-chloro-2-fluorobenzyl) oxy)-5-fluoropyrimidin-2-yl)- 2-fluorobenzyl)-1-(oxetan-2-ylmethyl)- 1H-benzo[d]imidazole-6-carboxylic acid |
| 18C | 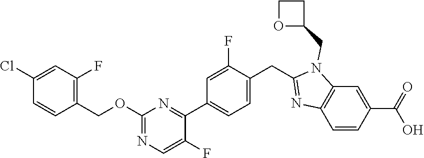 [SMILES: O=C(O)c1ccc2nc(Cc3ccc(-c4nc(OCc5ccc(Cl)cc5F)ncc4F)cc3F)n(C[C@@H]3CCO3)c2c1] | (S)-2-(4-(2-(4-chloro-2-fluorobenzyloxy)- 5-fluoropyrimidin-4-yl)-2-fluorobenzyl)-1- (oxetan-2-ylmethyl)-1H- benzo[d]imidazole-6-carboxylic acid |
| 19C | 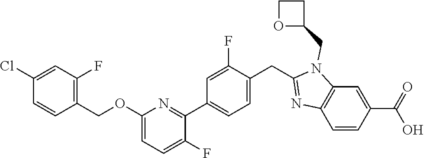 [SMILES: O=C(O)c1ccc2nc(Cc3ccc(-c4nc(OCc5ccc(Cl)cc5F)ccc4F)cc3F)n(C[C@@H]3CCO3)c2c1] | (S)-2-(4-(6-((4-chloro-2- fluorobenzyl)oxy)-3-fluoropyridin-2-yl)-2- fluorobenzyl)-1-(oxetan-2-ylmethyl)-1H- benzo[d]imidazole-6-carboxylic acid |
| 20C | 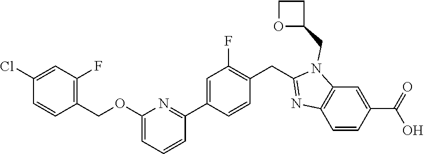 [SMILES: O=C(O)c1ccc2nc(Cc3ccc(-c4cccc(OCc5ccc(Cl)cc5F)n4)cc3F)n(C[C@@H]3CCO3)c2c1] | (S)-2-(4-(6-((4-chloro-2- fluorobenzyl)oxy)pyridin-2-yl)-2- fluorobenzyl)-1-(oxetan-2-ylmethyl)-1H- benzo[d]imidazole-6-carboxylic acid |
| 21C | 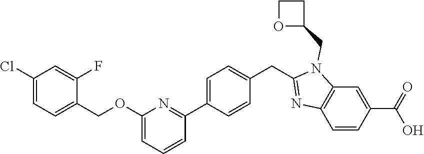 [SMILES: O=C(O)c1ccc2nc(Cc3ccc(-c4cccc(OCc5ccc(Cl)cc5F)n4)cc3)n(C[C@@H]3CCO3)c2c1] | (S)-2-(4-(6-((4-chloro-2- fluorobenzyl)oxy)pyridin-2-yl)benzyl)-1- (oxetan-2-ylmethyl)-1H- benzo[d]imidazole-6-carboxylic acid |
| 22C | 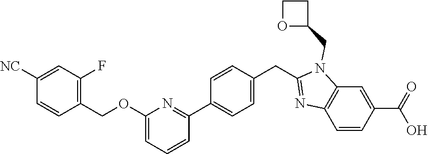 [SMILES: N#Cc1ccc(COc2cccc(-c3ccc(Cc4nc5ccc(C(=O)O)cc5n4C[C@@H]4CCO4)cc3)n2)c(F)c1] | (S)-2-(4-(6-((4-cyano-2- fluorobenzyl)oxy)pyridin-2-yl)benzyl)-1- (oxetan-2-ylmethyl)-1H- benzo[d]imidazole-6-carboxylic acid |
| 23C | 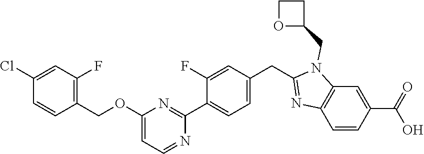 [SMILES: O=C(O)c1ccc2nc(Cc3ccc(-c4nccc(OCc5ccc(Cl)cc5F)n4)c(F)c3)n(C[C@@H]3CCO3)c2c1] | (S)-2-(4-(4-((4-chloro-2- fluorobenzyl)oxy)pyrimidin-2-yl)-3- fluorobenzyl)-1-(oxetan-2-ylmethyl)-1H- benzo[d]imidazole-6-carboxylic acid |
| 24C | 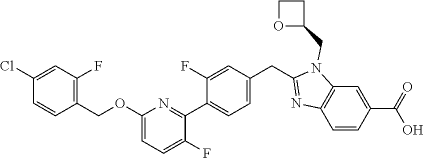 [SMILES: O=C(O)c1ccc2nc(Cc3ccc(-c4nc(OCc5ccc(Cl)cc5F)ccc4F)c(F)c3)n(C[C@@H]3CCO3)c2c1] | (S)-2-(4-(6-(4-chloro-2-fluorobenzyloxy)- 3-fluoropyridin-2-yl)-3-fluorobenzyl)-1- (oxetan-2-ylmethyl)-1H- benzo[d]imidazole-6-carboxylic acid |
| 25C | 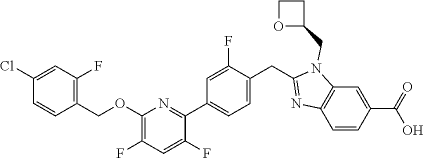 [SMILES: O=C(O)c1ccc2nc(Cc3ccc(-c4nc(OCc5ccc(Cl)cc5F)c(F)cc4F)cc3F)n(C[C@@H]3CCO3)c2c1] | (S)-2-(4-(6-(4-chloro-2-fluorobenzyloxy)- 3,5-difluoropyridin-2-yl)-2-fluorobenzyl)- 3-(oxetan-2-ylmethyl)-3H- benzo[d]imidazole-5-carboxylic acid |
| 26C | 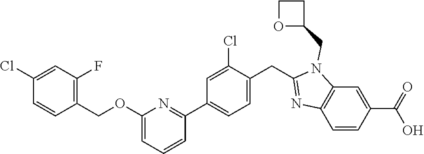 [SMILES: O=C(O)c1ccc2nc(Cc3ccc(-c4cccc(OCc5ccc(Cl)cc5F)n4)cc3Cl)n(C[C@@H]3CCO3)c2c1] | (S)-2-(2-chloro-4-(6-((4-chloro-2- fluorobenzyl)oxy)pyridin-2-yl)benzyl)-1- (oxetan-2-ylmethyl)-1H- benzo[d]imidazole-6-carboxylic acid |
| 27C | 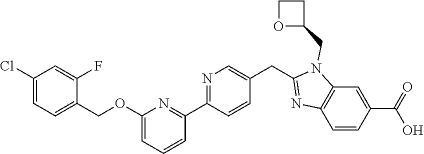 [SMILES: O=C(O)c1ccc2nc(Cc3ccc(-c4cccc(OCc5ccc(Cl)cc5F)n4)nc3)n(C[C@@H]3CCO3)c2c1] | (S)-2-((6′-(4-chloro-2-fluorobenzyloxy)- 2,2′-bipyridin-5-yl)methyl)-1-(oxetan-2- ylmethyl)-1H-benzo[d]imidazole-6- carboxylic acid |
| 28C | 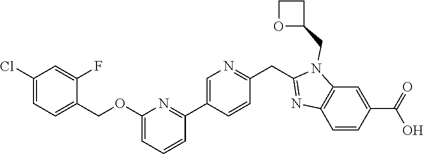 [SMILES: O=C(O)c1ccc2nc(Cc3ccc(-c4cccc(OCc5ccc(Cl)cc5F)n4)cn3)n(C[C@@H]3CCO3)c2c1] | (S)-2-((6-((4-chloro-2-fluorobenzyl)oxy)- [2,3′-bipyridin]-6′-yl)methyl)-1-(oxetan-2- ylmethyl)-1H-benzo[d]imidazole-6- carboxylic acid |
| 29C | 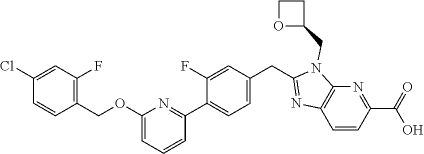 [SMILES: O=C(O)c1ccc2nc(Cc3ccc(-c4cccc(OCc5ccc(Cl)cc5F)n4)c(F)c3)n(C[C@@H]3CCO3)c2n1] | (S)-2-(4-(6-((4-chloro-2- fluorobenzyl)oxy)pyridin-2-yl)-3- fluorobenzyl)-3-(oxetan-2-ylmethyl)-3H- imidazo[4,5-b]pyridine-5-carboxylic acid |
| 30C | 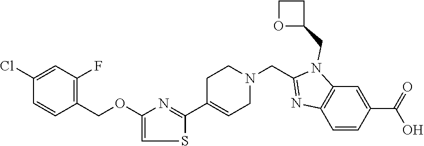 [SMILES: O=C(O)c1ccc2nc(CN3CC=C(c4nc(OCc5ccc(Cl)cc5F)cs4)CC3)n(C[C@@H]3CCO3)c2c1] | (S)-2-((4-(4-((4-chloro-2- fluorobenzyl)oxy)thiazol-2-yl)-3,6- dihydropyridin-1(2H)-yl)methyl)-1- (oxetan-2-ylmethyl)-1H- benzo[d]imidazole-6-carboxylic acid |
| 31C | 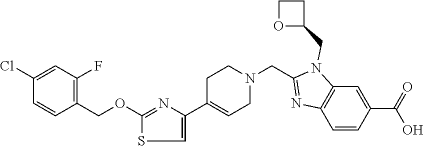 [SMILES: O=C(O)c1ccc2nc(CN3CC=C(c4csc(OCc5ccc(Cl)cc5F)n4)CC3)n(C[C@@H]3CCO3)c2c1] | (S)-2-((4-(2-((4-chloro-2- fluorobenzyl)oxy)thiazol-4-yl)-3,6- dihydropyridin-1(2H)-yl)methyl)-1- (oxetan-2-ylmethyl)-1H- benzo[d]imidazole-6-carboxylic acid |
| 32C | 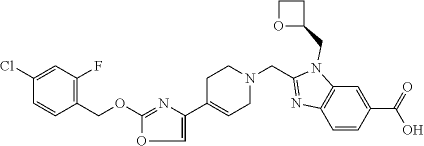 [SMILES: O=C(O)c1ccc2nc(CN3CC=C(c4coc(OCc5ccc(Cl)cc5F)n4)CC3)n(C[C@@H]3CCO3)c2c1] | (S)-2-((4-(2-((4-chloro-2- fluorobenzyl)oxy)oxazol-4-yl)-5,6- dihydropyridin-1(2H)-yl)methyl)-1- (oxetan-2-ylmethyl)-1H- benzo[d]imidazole-6-carboxylic acid |
| 33C | 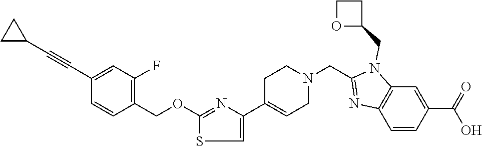 [SMILES: O=C(O)c1ccc2nc(CN3CC=C(c4csc(OCc5ccc(C#CC6CC6)cc5F)n4)CC3)n(C[C@@H]3CCO3)c2c1] | (S)-2-((4-(2-((4-(cyclopropylethynyl)-2- fluorobenzyl)oxy)thiazol-4-yl)-3,6- dihydropyridin-1(2H)yl) methyl)-1-(oxetan-2-ylmethyl)-1H- benzo[d]imidazole-6-carboxylic acid |
| 34C | 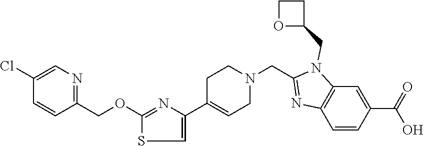 [SMILES: O=C(O)c1ccc2nc(CN3CC=C(c4csc(OCc5ccc(Cl)cn5)n4)CC3)n(C[C@@H]3CCO3)c2c1] | (S)-2-((4-(2-((5-chloropyridin-2- yl)methoxy)thiazol-4-yl)-3,6- dihydropyridin-1(2H)-yl)methyl)-1- (oxetan-2-ylmethyl)-1H- benzo[d]imidazole-6-carboxylic acid |
| 35C | 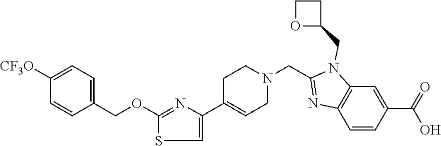 | (S)-1-(oxetan-2-ylmethyl)-2-((4-(2-((4- (trifluoromethoxy)benzyl)oxy)thiazol-4- yl)-3,6-dihydropyridin-1(2H)-yl)methyl)- 1H-benzo[d]imidazole-6-carboxylic acid |
| 36C | 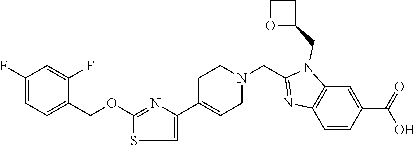 [SMILES: O=C(O)c1ccc2nc(CN3CC=C(c4csc(OCc5ccc(F)cc5F)n4)CC3)n(C[C@@H]3CCO3)c2c1] | (S)-2-((4-(2-((2,4- difluorobenzyl)oxy)thiazol-4-yl)-3,6- dihydropyridin-1(2H)-yl)methyl)-1- (oxetan-2-ylmethyl)-1H- benzo[d]imidazole-6-carboxylic acid |
| 37C | 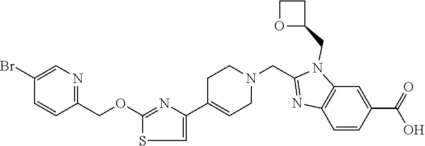 [SMILES: O=C(O)c1ccc2nc(CN3CC=C(c4csc(OCc5ccc(Br)cn5)n4)CC3)n(C[C@@H]3CCO3)c2c1] | (S)-2-((4-(2-((5-bromopyridin-2- yl)methoxy)thiazol-4-yl)-3,6- dihydropyridin-1(2H)-yl)methyl)-1- (oxetan-2-ylmethyl)-1H- benzo[d]imidazole-6-carboxylic acid |
| 38C | 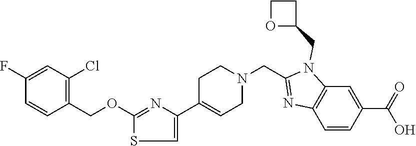 [SMILES: O=C(O)c1ccc2nc(CN3CC=C(c4csc(OCc5ccc(F)cc5Cl)n4)CC3)n(C[C@@H]3CCO3)c2c1] | (S)-2-((4-(2-((2-chloro-4- fluorobenzyl)oxy)thiazol-4-yl)-3,6- dihydropyridin-1(2H)-yl)methyl)-1- (oxetan-2-ylmethyl)-1H- benzo[d]imidazole-6-carboxylic acid |
| 39C | 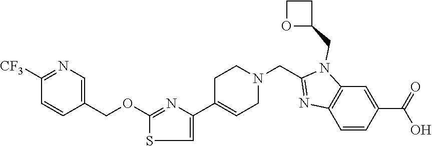 [SMILES: O=C(O)c1ccc2nc(CN3CC=C(c4csc(OCc5ccc(C(F)(F)F)nc5)n4)CC3)n(C[C@@H]3CCO3)c2c1] | (S)-1-(oxetan-2-ylmethyl)-2-((4-(2-((6- (trifluoromethyl)pyridin-3- yl)methoxy)thiazol-4-yl)-3,6- dihydropyridin-1(2H)-yl)methyl)-1H- benzo[d]imidazole-6-carboxylic acid |
| 40C | 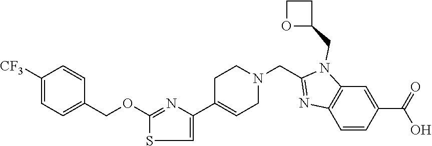 [SMILES: O=C(O)c1ccc2nc(CN3CC=C(c4csc(OCc5ccc(C(F)(F)F)cc5)n4)CC3)n(C[C@@H]3CCO3)c2c1] | (S)-1-(oxetan-2-ylmethyl)-2-((4-(2-((4- (trifluoromethyl)benzyl)oxy)thiazol-4-yl)- 3,6-dihydropyridin-1(2H)-yl)methyl)-1H- benzo[d]imidazole-6-carboxylic acid |
| 41C | 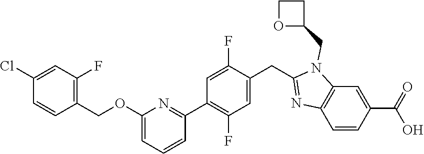 [SMILES: O=C(O)c1ccc2nc(Cc3cc(F)c(-c4cccc(OCc5ccc(Cl)cc5F)n4)cc3F)n(C[C@@H]3CCO3)c2c1] | (S)-2-(4-(6-((4-chloro-2- fluorobenzyl)oxy)pyridin-2-yl)-2,5- difluorobenzyl)-1-(oxetan-2-ylmethyl)- 1H-benzo[d]imidazole-6-carboxylic acid |
| 42C | 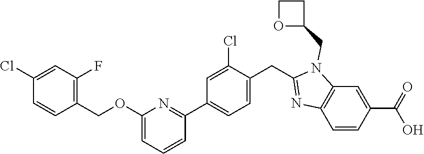 [SMILES: O=C(O)c1ccc2nc(Cc3ccc(-c4cccc(OCc5ccc(Cl)cc5F)n4)cc3Cl)n(C[C@@H]3CCO3)c2c1] | (S)-2-(2-chloro-4-(6-((4-chloro-2- fluorobenzyl)oxy)pyridin-2-yl)benzyl)-1- (oxetan-2-ylmethyl)-1H- benzo[d]imidazole-6-carboxylic acid |
| 43C | 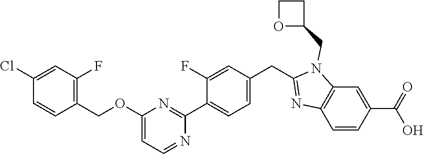 [SMILES: O=C(O)c1ccc2nc(Cc3ccc(-c4nccc(OCc5ccc(Cl)cc5F)n4)c(F)c3)n(C[C@@H]3CCO3)c2c1] | (S)-2-(4-(4-((4-chloro-2- fluorobenzyl)oxy)pyrimidin-2-yl)-3- fluorobenzyl)-1-(oxetan-2-ylmethyl)-1H- benzo[d]imidazole-6-carboxylic acid |
| 44C | 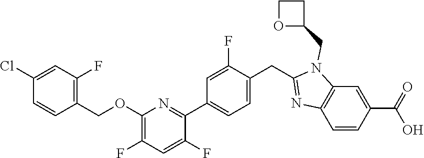 [SMILES: O=C(O)c1ccc2nc(Cc3ccc(-c4nc(OCc5ccc(Cl)cc5F)c(F)cc4F)cc3F)n(C[C@@H]3CCO3)c2c1] | (S)-2-(4-(6-(4-chloro-2-fluorobenzyloxy)- 3,5-difluoropyridin-2-yl)-2-fluorobenzyl)- 1-(oxetan-2-ylmethyl)-1H- benzo[d]imidazole-6-carboxylic acid |
| 45C | 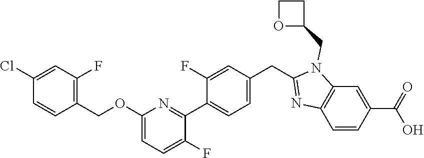 [SMILES: O=C(O)c1ccc2nc(Cc3ccc(-c4nc(OCc5ccc(Cl)cc5F)ccc4F)c(F)c3)n(C[C@@H]3CCO3)c2c1] | (S)-2-(4-(6-(4-chloro-2-fluorobenzyloxy)- 3-fluoropyridin-2-yl)-3-fluorobenzyl)-1- (oxetan-2-ylmethyl)-1H- benzo[d]imidazole-6-carboxylic acid |
| 46C | 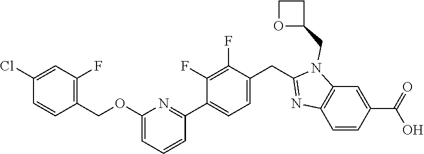 [SMILES: O=C(O)c1ccc2nc(Cc3ccc(-c4cccc(OCc5ccc(Cl)cc5F)n4)c(F)c3F)n(C[C@@H]3CCO3)c2c1] | (S)-2-(4-(6-(4-chloro-2- fluorobenzyloxy)pyridin-2-yl)-2,3- difluorobenzyl)-1-(oxetan-2-ylmethyl)- 1H-benzo[d]imidazole-6-carboxylic acid |
| 47C | 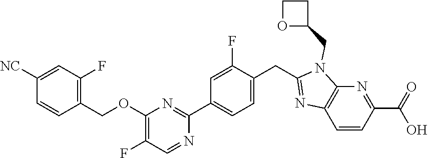 [SMILES: N#Cc1ccc(COc2nc(-c3ccc(Cc4nc5ccc(C(=O)O)nc5n4C[C@@H]4CCO4)c(F)c3)ncc2F)c(F)c1] | (S)-2-(4-(4-(4-cyano-2-fluorobenzyloxy)- 5-fluoropyrimidin-2-yl)-2-fluorobenzyl)-3- (oxetan-2-ylmethyl)-3H-imidazo[4,5- b]pyridine-5-carboxylic acid |
| 48C | 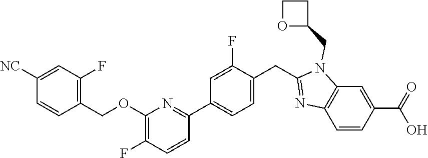 [SMILES: N#Cc1ccc(COc2nc(-c3ccc(Cc4nc5ccc(C(=O)O)cc5n4C[C@@H]4CCO4)c(F)c3)ccc2F)c(F)c1] | (S)-2-(4-(6-((4-cyano-2- fluorobenzyl)oxy)-5-fluoropyridin-2-yl)-2- fluorobenzyl)-1-(oxetan-2-ylmethyl)-1H- benzo[d]imidazole-6-carboxylic acid |
| 49C | 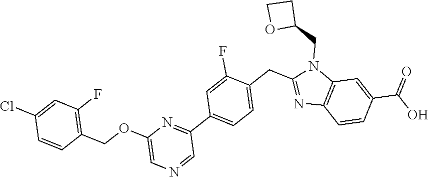 [SMILES: O=C(O)c1ccc2nc(Cc3ccc(-c4cncc(OCc5ccc(Cl)cc5F)n4)cc3F)n(C[C@@H]3CCO3)c2c1] | (S)-2-(4-(6-((4-chloro-2- fluorobenzyl)oxy)pyrazin-2-yl)-2- fluorobenzyl)-1-(oxetan-2-ylmethyl)-1H- benzo[d]imidazole-6-carboxylic acid |
| 50C | 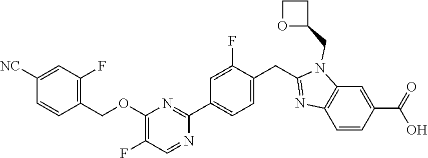 [SMILES: N#Cc1ccc(COc2nc(-c3ccc(Cc4nc5ccc(C(=O)O)cc5n4C[C@@H]4CCO4)c(F)c3)ncc2F)c(F)c1] | (S)-2-(4-(4-((4-cyano-2- fluorobenzyl)oxy)-5-fluoropyrimidin-2-yl)- 2-fluorobenzyl)-1-(oxetan-2-ylmethyl)- 1H-benzo[d]imidazole-6-carboxylic acid |
| 51C | 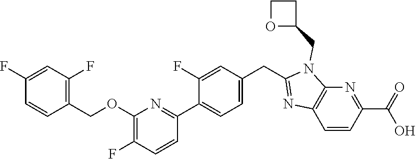 [SMILES: O=C(O)c1ccc2nc(Cc3ccc(-c4ccc(F)c(OCc5ccc(F)cc5F)n4)c(F)c3)n(C[C@@H]3CCO3)c2n1] | (S)-2-(4-(6-(2,4-difluorobenzyloxy)-5- fluoropyridin-2-yl)-3-fluorobenzyl)-3- (oxetan-2-ylmethyl)-3H-imidazo[4,5- b]pyridine-5-carboxylic acid |
| 52C | 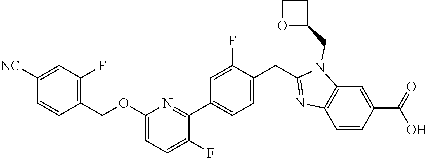 [SMILES: N#Cc1ccc(COc2ccc(F)c(-c3ccc(Cc4nc5ccc(C(=O)O)cc5n4C[C@@H]4CCO4)c(F)c3)n2)c(F)c1] | (S)-2-(4-(6-((4-cyano-2- fluorobenzyl)oxy)-3-fluoropyridin-2-yl)-2- fluorobenzyl)-1-(oxetan-2-ylmethyl)-1H- benzo[d]imidazole-6-carboxylic acid |
| 53C | 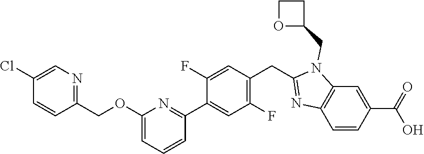 [SMILES: O=C(O)c1ccc2nc(Cc3cc(F)c(-c4cccc(OCc5ccc(Cl)cn5)n4)cc3F)n(C[C@@H]3CCO3)c2c1] | (S)-2-(4-(6-((5-chloropyridin-2- yl)methoxy)pyridin-2-yl)-2,5- difluorobenzyl)-1-(oxetan-2-ylmethyl)- 1H-benzo[d]imidazole-6-carboxylic acid |
| 54C | 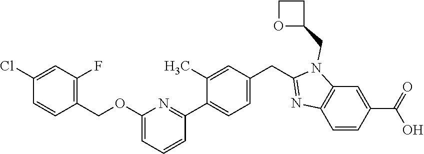 [SMILES: Cc1cc(Cc2nc3ccc(C(=O)O)cc3n2C[C@@H]2CCO2)ccc1-c1cccc(OCc2ccc(Cl)cc2F)n1] | (S)-2-(4-(6-((4-chloro-2- fluorobenzyl)oxy)pyridin-2-yl)-3- methylbenzyl)-1-(oxetan-2-ylmethyl)- 1Hbenzo[d]imidazole-6-carboxylic acid |
| 55C | 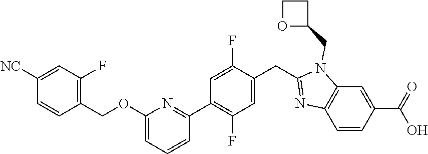 [SMILES: N#Cc1ccc(COc2cccc(-c3cc(F)c(Cc4nc5ccc(C(=O)O)cc5n4C[C@@H]4CCO4)cc3F)n2)c(F)c1] | (S)-2-(4-(6-((4-cyano-2- fluorobenzyl)oxy)pyridin-2-yl)-2,5- difluorobenzyl)-1-(oxetan-2-ylmethyl)- 1H-benzo[d]imidazole-6-carboxylic acid |
| 56C | 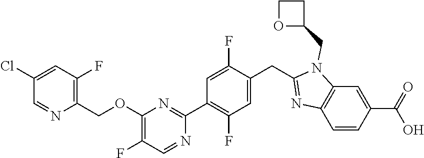 [SMILES: O=C(O)c1ccc2nc(Cc3cc(F)c(-c4ncc(F)c(OCc5ncc(Cl)cc5F)n4)cc3F)n(C[C@@H]3CCO3)c2c1] | (S)-2-(4-(4-((5-chloro-3-fluoropyridin-2- yl)methoxy)-5-fluoropyrimidin-2-yl)-2,5- difluorobenzyl)-1-(oxetan-2-ylmethyl)- 1H-benzo[d]imidazole-6-carboxylic acid |
| 57C | 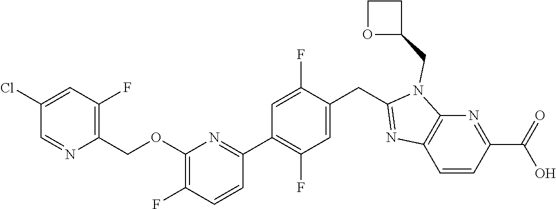 [SMILES: O=C(O)c1ccc2nc(Cc3cc(F)c(-c4ccc(F)c(OCc5ncc(Cl)cc5F)n4)cc3F)n(C[C@@H]3CCO3)c2n1] | (S)-2-(4-(4-((5-chloro-3-fluoropyridin-2- yl)methoxy)-5-fluoropyrimidin-2-yl)-2,5- difluorobenzyl)-3-(oxetan-2-ylmethyl)- 3H-imidazo[4,5-b]pyridine-5-carboxylic acid |
| 58C | 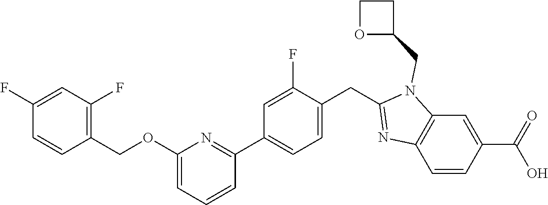 [SMILES: O=C(O)c1ccc2nc(Cc3ccc(-c4cccc(OCc5ccc(F)cc5F)n4)cc3F)n(C[C@@H]3CCO3)c2c1] | (S)-2-(4-(6-((2,4- difluorobenzyl)oxy)pyridin-2-yl)-2- fluorobenzyl)-1-(oxetan-2-ylmethyl)-1H- benzo[d]imidazole-6-carboxylic acid |
| 59C | 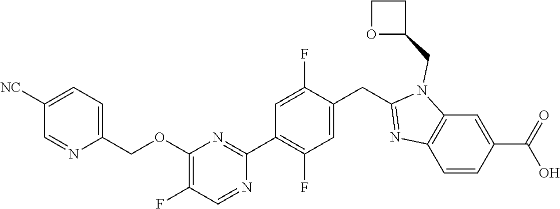 [SMILES: N#Cc1ccc(COc2nc(-c3cc(F)c(Cc4nc5ccc(C(=O)O)cc5n4C[C@@H]4CCO4)cc3F)ncc2F)nc1] | (S)-2-(4-(4-((5-cyanopyridin-2- yl)methoxy)-5-fluoropyrimidin-2-yl)-2,5- difluorobenzyl)-1-(oxetan-2-ylmethyl)- 1H-benzo[d]imidazole-6-carboxylic acid |
| 60C | 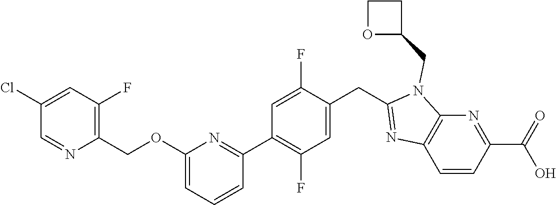 [SMILES: O=C(O)c1ccc2nc(Cc3cc(F)c(-c4cccc(OCc5ncc(Cl)cc5F)n4)cc3F)n(C[C@@H]3CCO3)c2n1] | (S)-2-(4-(6-((5-chloro-3-fluoropyridin-2- yl)methoxy)pyridin-2-yl)-2,5- difluorobenzyl)-3-(oxetan-2-ylmethyl)- 3H-imidazo[4,5-b]pyridine-5-carboxylic acid |
| 61C | 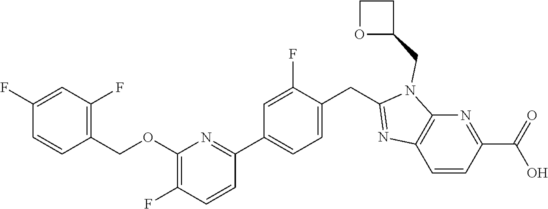 [SMILES: O=C(O)c1ccc2nc(Cc3ccc(-c4ccc(F)c(OCc5ccc(F)cc5F)n4)cc3F)n(C[C@@H]3CCO3)c2n1] | (S)-2-(4-(6-(2,4-difluorobenzyloxy)-5- fluoropyridin-2-yl)-2-fluorobenzyl)-3- (oxetan-2-ylmethyl)-3H-imidazo[4,5- b]pyridine-5-carboxylic acid |
| 62C | 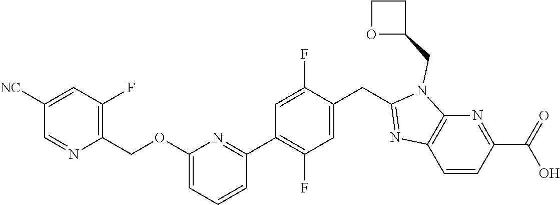 [SMILES: N#Cc1cnc(COc2cccc(-c3cc(F)c(Cc4nc5ccc(C(=O)O)nc5n4C[C@@H]4CCO4)cc3F)n2)c(F)c1] | (S)-2-(4-(6-((5-cyano-3-fluoropyridin-2- yl)methoxy)pyridin-2-yl)-2,5- difluorobenzyl)-3-(oxetan-2-ylmethyl)- 3H-imidazo[4,5-b]pyridine-5-carboxylic acid |
| 63C | 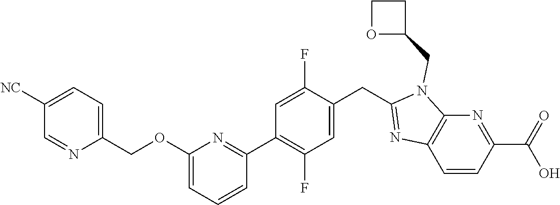 [SMILES: N#Cc1ccc(COc2cccc(-c3cc(F)c(Cc4nc5ccc(C(=O)O)nc5n4C[C@@H]4CCO4)cc3F)n2)nc1] | (S)-2-(4-(6-((5-cyanopyridin-2- yl)methoxy)pyridin-2-yl)-2,5- difluorobenzyl)-3-(oxetan-2-ylmethyl)- 3H-imidazo[4,5-b]pyridine-5-carboxylic acid |
| 64C | 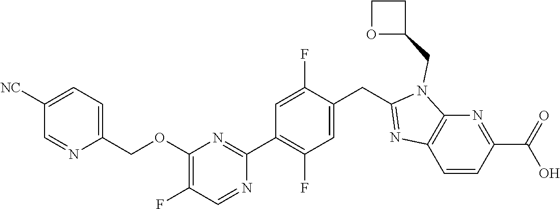 [SMILES: N#Cc1ccc(COc2nc(-c3cc(F)c(Cc4nc5ccc(C(=O)O)nc5n4C[C@@H]4CCO4)cc3F)ncc2F)nc1] | (S)-2-(4-(4-((5-cyanopyridin-2- yl)methoxy)-5-fluoropyrimidin-2-yl)-2,5- difluorobenzyl)-3-(oxetan-2-ylmethyl)- 3H-imidazo[4,5-b]pyridine-5-carboxylic acid |
| 65C | 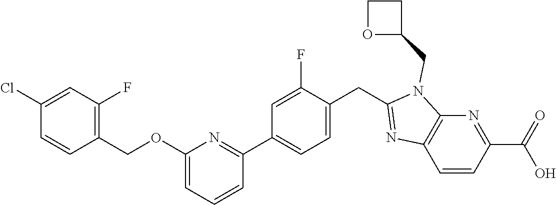 [SMILES: O=C(O)c1ccc2nc(Cc3ccc(-c4cccc(OCc5ccc(Cl)cc5F)n4)cc3F)n(C[C@@H]3CCO3)c2n1] | 2-[[4-[6-[(4-chloro-2-fluoro- phenyl)methoxy]-2-pyridyl]-2-fluoro- phenyl]methyl]-3-[(2S)-oxetan-2- yl]methyl]imidazo[4,5-b]pyridine-5- carboxylic acid |
| 66C | 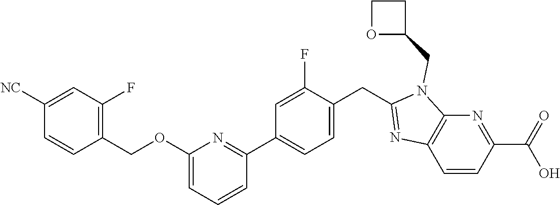 [SMILES: N#Cc1ccc(COc2cccc(-c3ccc(Cc4nc5ccc(C(=O)O)nc5n4C[C@@H]4CCO4)c(F)c3)n2)c(F)c1] | (S)-2-(4-(6-((4-cyano-2- fluorobenzyl)oxy)pyridin-2-yl)-2- fluorobenzyl)-3-(oxetan-2-ylmethyl)-3H- imidazo[4,5-b]pyridine-5-carboxylic acid |
| 67C | 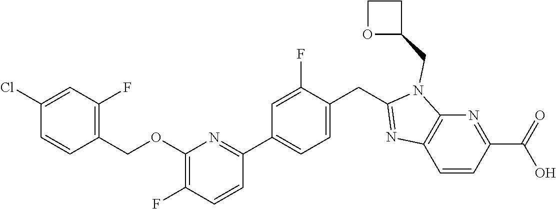 [SMILES: O=C(O)c1ccc2nc(Cc3ccc(-c4ccc(F)c(OCc5ccc(Cl)cc5F)n4)cc3F)n(C[C@@H]3CCO3)c2n1] | (S)-2-(4-(6-(4-chloro-2-fluorobenzyloxy)- 5-fluoropyridin-2-yl)-2-fluorobenzyl)-3- (oxetan-2-ylmethyl)-3H-imidazo[4,5- b]pyridine-5-carboxylic acid |
| 68C | 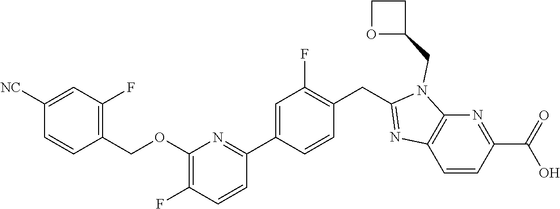 [SMILES: N#Cc1ccc(COc2nc(-c3ccc(Cc4nc5ccc(C(=O)O)nc5n4C[C@@H]4CCO4)c(F)c3)ccc2F)c(F)c1] | (S)-2-(4-(6-(4-cyano-2-fluorobenzyloxy)- 5-fluoropyridin-2-yl)-2-fluorobenzyl)-3- (oxetan-2-ylmethyl)-3H-imidazo[4,5- b]pyridine-5-carboxylic acid |
| 69C | 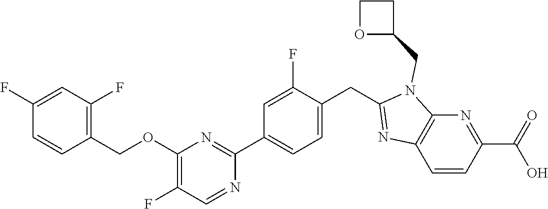 [SMILES: O=C(O)c1ccc2nc(Cc3ccc(-c4ncc(F)c(OCc5ccc(F)cc5F)n4)cc3F)n(C[C@@H]3CCO3)c2n1] | (S)-2-(4-(4-((2,4-difluorobenzyl)oxy)-5- fluoropyrimidin-2-yl)-2-fluorobenzyl)-3- (oxetan-2-ylmethyl)-3H-imidazo[4,5- b]pyridine-5-carboxylic acid |
| 70C | 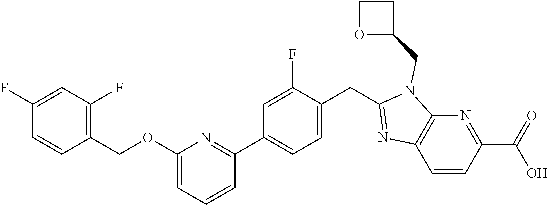 [SMILES: O=C(O)c1ccc2nc(Cc3ccc(-c4cccc(OCc5ccc(F)cc5F)n4)cc3F)n(C[C@@H]3CCO3)c2n1] | (S)-2-(4-(6-((2,4- difluorobenzyl)oxy)pyridin-2-yl)-2- fluorobenzyl)-3-(oxetan-2-ylmethyl)- 3Himidazo[4,5-b]pyridine-5-carboxylic acid |
| 71C | 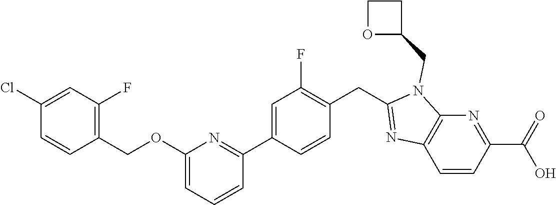 [SMILES: O=C(O)c1ccc2nc(Cc3ccc(-c4cccc(OCc5ccc(Cl)cc5F)n4)cc3F)n(C[C@@H]3CCO3)c2n1] | (S)-2-(4-(6-((4-chloro-2- fluorobenzyl)oxy)pyridin-2-yl)-2- fluorobenzyl)-3-(oxetan-2-ylmethyl)- 3Himidazo[4,5-b]pyridine-5-carboxylic acid |
| 72C | 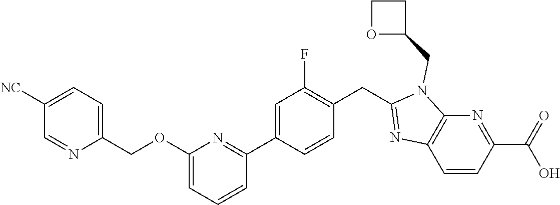 [SMILES: N#Cc1ccc(COc2cccc(-c3ccc(Cc4nc5ccc(C(=O)O)nc5n4C[C@@H]4CCO4)c(F)c3)n2)nc1] | (S)-2-(4-(6-((5-cyanopyridin-2- yl)methoxy)pyridin-2-yl)-2-fluorobenzyl)- 3-(oxetan-2-ylmethyl)-3Himidazo[4,5- b]pyridine-5-carboxylic acid |
| 73C | 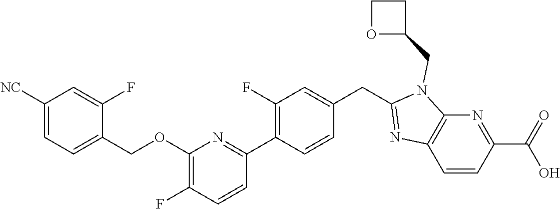 [SMILES: N#Cc1ccc(COc2nc(-c3ccc(Cc4nc5ccc(C(=O)O)nc5n4C[C@@H]4CCO4)cc3F)ccc2F)c(F)c1] | (S)-2-(4-(6-((4-cyano-2- fluorobenzyl)oxy)-5-fluoropyridin-2-yl)-3- fluorobenzyl)-3-(oxetan-2-ylmethyl)-3H- imidazo[4,5-b]pyridine-5-carboxylic acid |
| 74C | 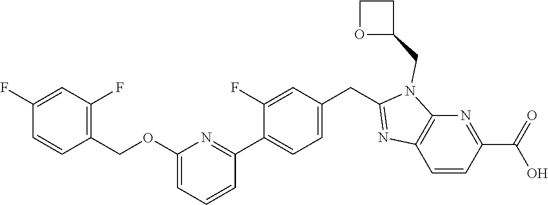 [SMILES: O=C(O)c1ccc2nc(Cc3ccc(-c4cccc(OCc5ccc(F)cc5F)n4)c(F)c3)n(C[C@@H]3CCO3)c2n1] | (S)-2-(4-(6-((2,4- difluorobenzyl)oxy)pyridin-2-yl)-3- fluorobenzyl)-3-(oxetan-2-ylmethyl)- 3Himidazo[4,5-b]pyridine-5-carboxylic acid |
| 75C | 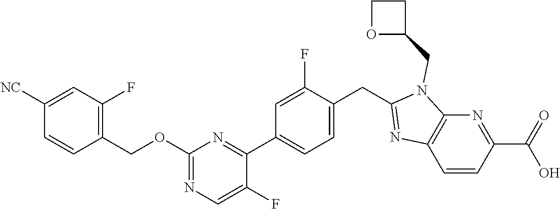 [SMILES: N#Cc1ccc(COc2ncc(F)c(-c3ccc(Cc4nc5ccc(C(=O)O)nc5n4C[C@@H]4CCO4)c(F)c3)n2)c(F)c1] | (S)-2-(4-(2-((4-cyano-2- fluorobenzyl)oxy)-5-fluoropyrimidin-4-yl)- 2-fluorobenzyl)-3-(oxetan-2-ylmethyl)- 3H-imidazo[4,5-b]pyridine-5-carboxylic acid |
| 76C | 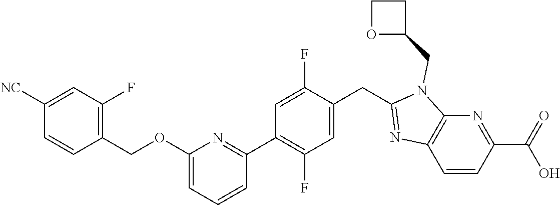 [SMILES: N#Cc1ccc(COc2cccc(-c3cc(F)c(Cc4nc5ccc(C(=O)O)nc5n4C[C@@H]4CCO4)cc3F)n2)c(F)c1] | (S)-2-(4-(6-((4-cyano-2- fluorobenzyl)oxy)pyridin-2-yl)-2,5- difluorobenzyl)-3-(oxetan-2-ylmethyl)- 3H-imidazo[4,5-b]pyridine-5-carboxylic acid |
| 77C | 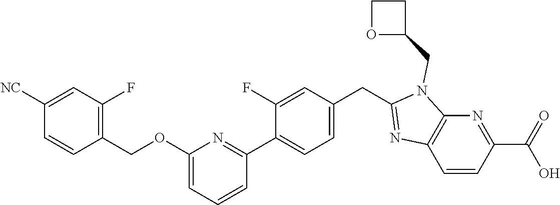 [SMILES: N#Cc1ccc(COc2cccc(-c3ccc(Cc4nc5ccc(C(=O)O)nc5n4C[C@@H]4CCO4)cc3F)n2)c(F)c1] | (S)-2-(4-(6-((4-cyano-2- fluorobenzyl)oxy)pyridin-2-yl)-3- fluorobenzyl)-3-(oxetan-2-ylmethyl)- 3Himidazo[4,5-b]pyridine-5-carboxylic acid |
| 78C | 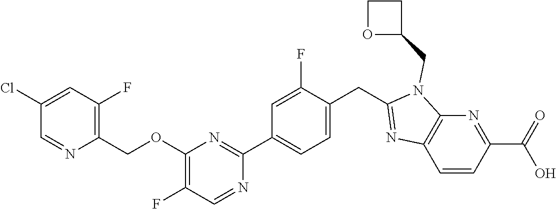 [SMILES: O=C(O)c1ccc2nc(Cc3ccc(-c4ncc(F)c(OCc5ncc(Cl)cc5F)n4)cc3F)n(C[C@@H]3CCO3)c2n1] | (S)-2-(4-(4-((5-chloro-3-fluoropyridin-2- yl)methoxy)-5-fluoropyrimidin-2-yl)-2- fluorobenzyl)-3-(oxetan-2-ylmethyl)-3H- imidazo[4,5-b]pyridine-5-carboxylic acid |
| 79C | 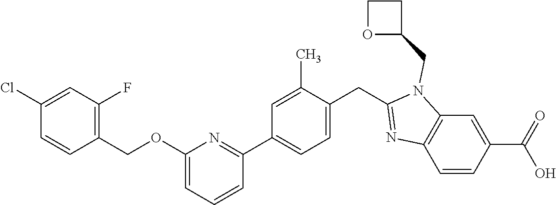 [SMILES: Cc1cc(-c2cccc(OCc3ccc(Cl)cc3F)n2)ccc1Cc1nc2ccc(C(=O)O)cc2n1C[C@@H]1CCO1] | (S)-2-(4-(6-((4-chloro-2- fluorobenzyl)oxy)pyridin-2-yl)-2- methylbenzyl)-1-(oxetan-2-ylmethyl)- 1Hbenzo[d]imidazole-6-carboxylic acid |
| 80C | 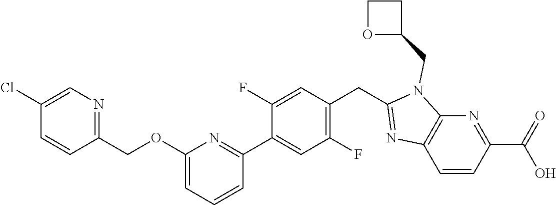 [SMILES: O=C(O)c1ccc2nc(Cc3cc(F)c(-c4cccc(OCc5ccc(Cl)cn5)n4)cc3F)n(C[C@@H]3CCO3)c2n1] | (S)-2-(4-(6-((5-chloropyridin-2- yl)methoxy)pyridin-2-yl)-2,5- difluorobenzyl)-3-(oxetan-2-ylmethyl)- 3H-imidazo[4,5-b]pyridine-5-carboxylic acid |
| 81C | 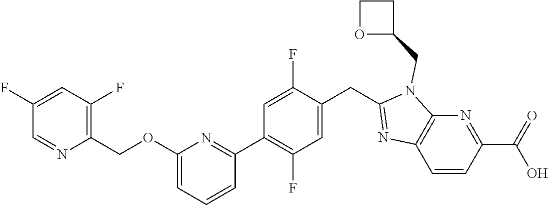 [SMILES: O=C(O)c1ccc2nc(Cc3cc(F)c(-c4cccc(OCc5ncc(F)cc5F)n4)cc3F)n(C[C@@H]3CCO3)c2n1] | (S)-2-(4-(6-((3,5-difluoropyridin-2- yl)methoxy)pyridin-2-yl)-2,5- difluorobenzyl)-3-(oxetan-2-ylmethyl)- 3H-imidazo[4,5-b]pyridine-5-carboxylic acid |
| 82C | 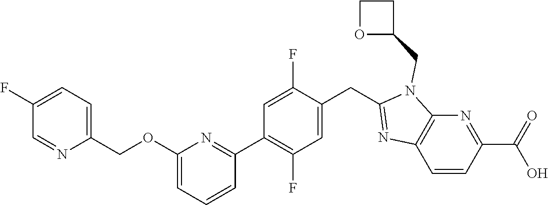 [SMILES: O=C(O)c1ccc2nc(Cc3cc(F)c(-c4cccc(OCc5ccc(F)cn5)n4)cc3F)n(C[C@@H]3CCO3)c2n1] | (S)-2-(2,5-difluoro-4-(6-((5-fluoropyridin- 2-yl)methoxy)pyridin-2-yl)benzyl)-3- (oxetan-2-ylmethyl)-3H-imidazo[4,5- b]pyridine-5-carboxylic acid |
| 83C | 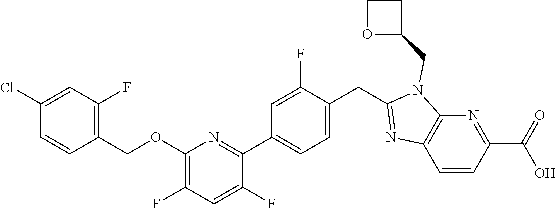 [SMILES: O=C(O)c1ccc2nc(Cc3ccc(-c4nc(OCc5ccc(Cl)cc5F)c(F)cc4F)cc3F)n(C[C@@H]3CCO3)c2n1] | (S)-2-(4-(6-(4-chloro-2-fluorobenzyloxy)- 3,5-difluoropyridin-2-yl)-2-fluorobenzyl)- 3-(oxetan-2-ylmethyl)-3H-imidazo[4,5- b]pyridine-5-carboxylic acid |
| 84C | 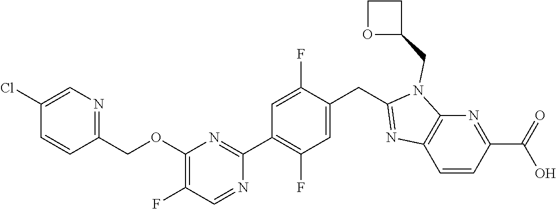 [SMILES: O=C(O)c1ccc2nc(Cc3cc(F)c(-c4ncc(F)c(OCc5ccc(Cl)cn5)n4)cc3F)n(C[C@@H]3CCO3)c2n1] | (S)-2-(4-(4-((5-chloropyridin-2- yl)methoxy)-5-fluoropyrimidin-2-yl)-2,5- difluorobenzyl)-3-(oxetan-2-ylmethyl)- 3H-imidazo[4,5-b]pyridine-5-carboxylic acid |
| 85C | 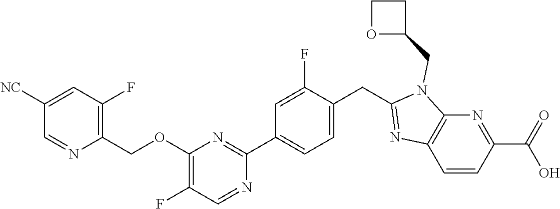 [SMILES: N#Cc1cnc(COc2nc(-c3ccc(Cc4nc5ccc(C(=O)O)nc5n4C[C@@H]4CCO4)c(F)c3)ncc2F)c(F)c1] | (S)-2-(4-(4-((5-cyano-3-fluoropyridin-2- yl)methoxy)-5-fluoropyrimidin-2-yl)-2- fluorobenzyl)-3-(oxetan-2-ylmethyl)-3H- imidazo[4,5-b]pyridine-5-carboxylic acid |
| 86C | 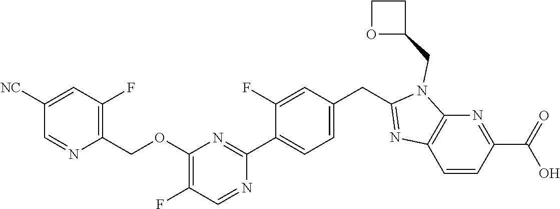 [SMILES: N#Cc1cnc(COc2nc(-c3ccc(Cc4nc5ccc(C(=O)O)nc5n4C[C@@H]4CCO4)cc3F)ncc2F)c(F)c1] | (S)-2-(4-(4-((5-cyano-3-fluoropyridin-2- yl)methoxy)-5-fluoropyrimidin-2-yl)-3- fluorobenzyl)-3-(oxetan-2-ylmethyl)-3H- imidazo[4,5-b]pyridine-5-carboxylic acid |
| 87C | 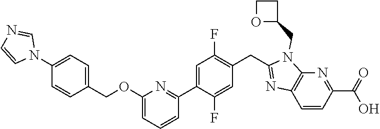 [SMILES: O=C(O)c1ccc2nc(Cc3cc(F)c(-c4cccc(OCc5ccc(-n6ccnc6)cc5)n4)cc3F)n(C[C@@H]3CCO3)c2n1] | (S)-2-(4-(6-(4-(JH-imidazol-1- yl)benzyloxy)pyridin-2-yl)-2,5- difluorobenzyl)-3-(oxetan-2-ylmethyl)- 3H-imidazo[4,5-b]pyridine-5-carboxylic acid |
| 88C | 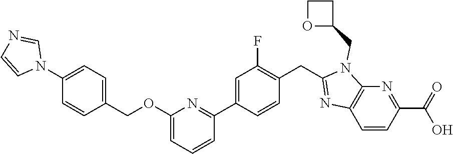 [SMILES: O=C(O)c1ccc2nc(Cc3ccc(-c4cccc(OCc5ccc(-n6ccnc6)cc5)n4)cc3F)n(C[C@@H]3CCO3)c2n1] | (S)-2-(4-(6-((4-(1H-imidazol-1- yl)benzyl)oxy)pyridin-2-yl)-2- fluorobenzyl)-3-(oxetan-2-ylmethyl)- 3H-imidazo[4,5-b]pyridine-5-carboxylic acid |
| 89C | 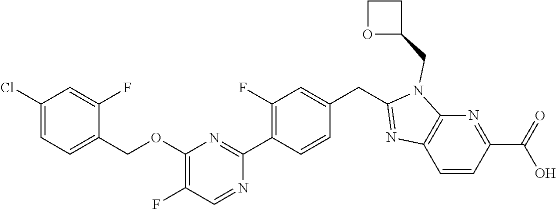 [SMILES: O=C(O)c1ccc2nc(Cc3ccc(-c4ncc(F)c(OCc5ccc(Cl)cc5F)n4)c(F)c3)n(C[C@@H]3CCO3)c2n1] | (S)-2-(4-(6-((4-chloro-2- fluorobenzyl)oxy)-5-fluoropyridin-2-yl)-3- fluorobenzyl)-3-(oxetan-2-ylmethyl)-3H- imidazo[4,5-b]pyridine-5-carboxylic acid |
| 90C | 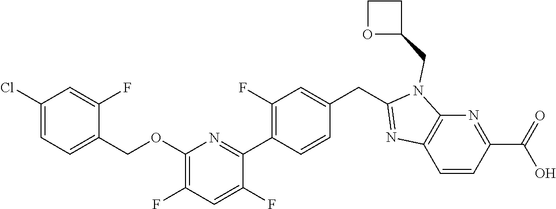 [SMILES: O=C(O)c1ccc2nc(Cc3ccc(-c4nc(OCc5ccc(Cl)cc5F)c(F)cc4F)c(F)c3)n(C[C@@H]3CCO3)c2n1] | (S)-2-(4-(6-((4-chloro-2- fluorobenzyl)oxy)-3,5-difluoropyridin-2- yl)-3-fluorobenzyl)-3-(oxetan-2- ylmethyl)-3H-imidazo[4,5-b]pyridine-5- carboxylic acid |
| 91C | 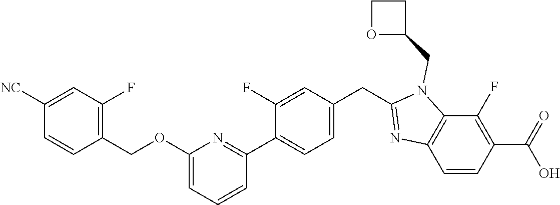 [SMILES: N#Cc1ccc(COc2cccc(-c3ccc(Cc4nc5ccc(C(=O)O)c(F)c5n4C[C@@H]4CCO4)cc3F)n2)c(F)c1] | (S)-2-(4-(6-((4-cyano-2- fluorobenzyl)oxy)pyridin-2-yl)-3- fluorobenzyl)-7-fluoro-1-(oxetan-2- ylmethyl)-1H-benzo[d]imidazole-6- carboxylic acid |
| 92C | 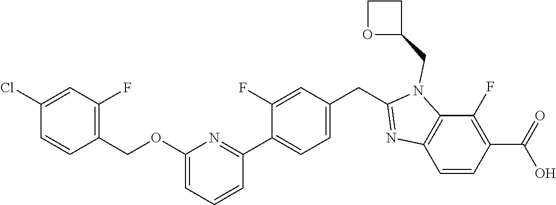 [SMILES: O=C(O)c1ccc2nc(Cc3ccc(-c4cccc(OCc5ccc(Cl)cc5F)n4)c(F)c3)n(C[C@@H]3CCO3)c2c1F] | (S)-2-(4-(6-((4-chloro-2- fluorobenzyl)oxy)pyridin-2-yl)-3- fluorobenzyl)-7-fluoro-1-(oxetan-2- ylmethyl)-1H-benzo[d]imidazole-6- carboxylic acid |
| 93C | 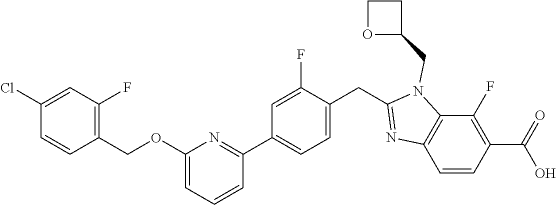 [SMILES: O=C(O)c1ccc2nc(Cc3ccc(-c4cccc(OCc5ccc(Cl)cc5F)n4)cc3F)n(C[C@@H]3CCO3)c2c1F] | (S)-2-(4-(6-((4-chloro-2- fluorobenzyl)oxy)pyridin-2-yl)-2- fluorobenzyl)-7-fluoro-1-(oxetan-2- ylmethyl)-1H-benzo[d]imidazole-6- carboxylic acid |
| 94C | 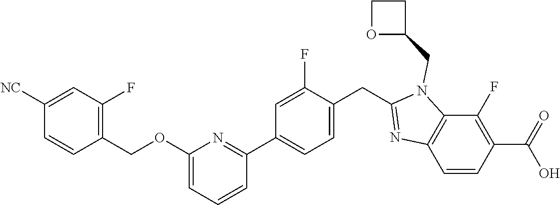 [SMILES: N#Cc1ccc(COc2cccc(-c3ccc(Cc4nc5ccc(C(=O)O)c(F)c5n4C[C@@H]4CCO4)c(F)c3)n2)c(F)c1] | (S)-2-(4-(6-((4-cyano-2- fluorobenzyl)oxy)pyridin-2-yl)-2- fluorobenzyl)-7-fluoro-1-(oxetan-2- ylmethyl)-1H-benzo[d]imidazole-6- carboxylic acid |
| 95C | 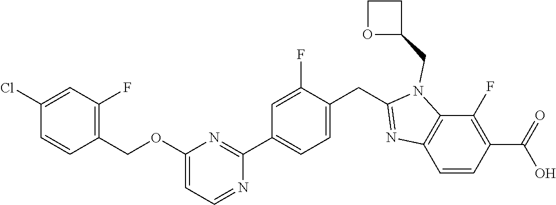 [SMILES: O=C(O)c1ccc2nc(Cc3ccc(-c4nccc(OCc5ccc(Cl)cc5F)n4)cc3F)n(C[C@@H]3CCO3)c2c1F] | (S)-2-(4-(4-((4-chloro-2- fluorobenzyl)oxy)pyrimidin-2-yl)-2- fluorobenzyl)-7-fluoro-1-(oxetan-2- ylmethyl)-1H-benzo[d]imidazole-6- carboxylic acid |
| 96C | 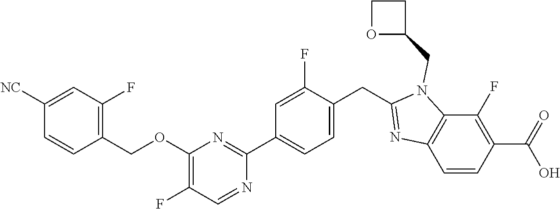 [SMILES: N#Cc1ccc(COc2nc(-c3ccc(Cc4nc5ccc(C(=O)O)c(F)c5n4C[C@@H]4CCO4)c(F)c3)ncc2F)c(F)c1] | (S)-2-(4-(4-((4-cyano-2- fluorobenzyl)oxy)-5-fluoropyrimidin-2-yl)- 2-fluorobenzyl)-7-fluoro-1-(oxetan-2- ylmethyl)-1H-benzo[d]imidazole-6- carboxylic acid |
| 97C | 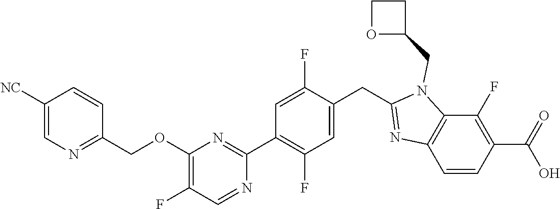 [SMILES: N#Cc1ccc(COc2nc(-c3cc(F)c(Cc4nc5ccc(C(=O)O)c(F)c5n4C[C@@H]4CCO4)cc3F)ncc2F)nc1] | (S)-2-(4-(4-((5-cyanopyridin-2- yl)methoxy)-5-fluoropyrimidin-2-yl)-2,5- difluorobenzyl)-7-fluoro-1-(oxetan-2- ylmethyl)-1H-benzo[d]imidazole-6- carboxylic acid |
| 98C | 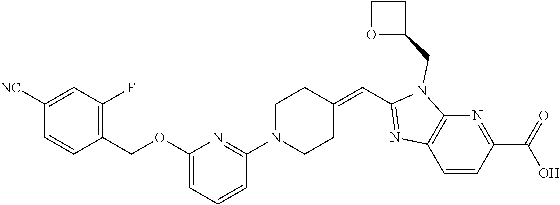 [SMILES: N#Cc1ccc(COc2cccc(N3CCC(=Cc4nc5ccc(C(=O)O)nc5n4C[C@@H]4CCO4)CC3)n2)c(F)c1] | (S)-2-((1-(6-(4-cyano-2- fluorobenzyloxy)pyridin-2-yl)piperidin-4- ylidene)methyl)-3-(oxetan-2-ylmethyl)- 3H-imidazo[4,5-b]pyridine-5-carboxylic acid |
| 99C | 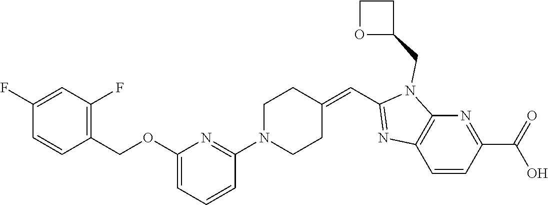 [SMILES: O=C(O)c1ccc2nc(C=C3CCN(c4cccc(OCc5ccc(F)cc5F)n4)CC3)n(C[C@@H]3CCO3)c2n1] | (S)-2-((1-(6-(2,4- difluorobenzyloxy)pyridin-2-yl)piperidin- 4-ylidene)methyl)-3-(oxetan-2-ylmethyl)- 3H-imidazo[4,5-b]pyridine-5-carboxylic acid |
| 100C | 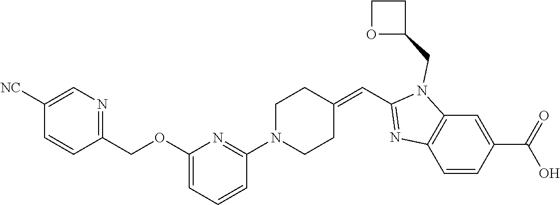 [SMILES: N#Cc1ccc(COc2cccc(N3CCC(=Cc4nc5ccc(C(=O)O)cc5n4C[C@@H]4CCO4)CC3)n2)nc1] | (S)-2-((1-(6-((5-cyanopyridin-2- yl)methoxy)pyridin-2-yl)piperidin-4- ylidene)methyl)-3-(oxetan-2-ylmethyl)- 3H-imidazo[4,5-b]pyridine-5-carboxylic acid |
| 101C | 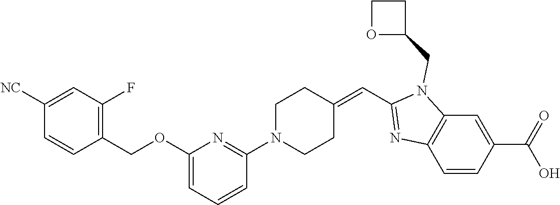 [SMILES: N#Cc1ccc(COc2cccc(N3CCC(=Cc4nc5ccc(C(=O)O)cc5n4C[C@@H]4CCO4)CC3)n2)c(F)c1] | (S)-2-((1-(6-((4-cyano-2- fluorobenzyl)oxy)pyridin-2-yl)piperidin-4- ylidene)methyl)-1-(oxetan-2-ylmethyl)- 1H-benzo[d]imidazole-6-carboxylic acid |
| 102C | 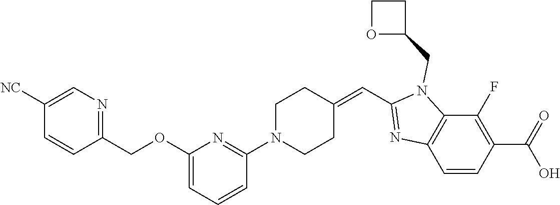 [SMILES: N#Cc1ccc(COc2cccc(N3CCC(=Cc4nc5ccc(C(=O)O)c(F)c5n4C[C@@H]4CCO4)CC3)n2)nc1] | (S)-2-((1-(6-((5-cyanopyridin-2- yl)methoxy)pyridin-2-yl)piperidin-4- ylidene)methyl)-7-fluoro-1-(oxetan-2- ylmethyl)-1H-benzo[d]imidazole-6- carboxylic acid |
| 103C | 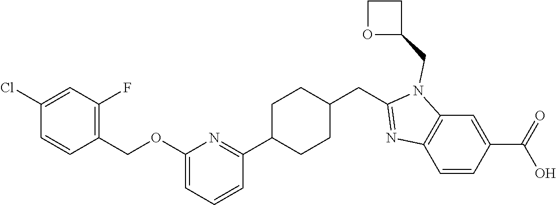 [SMILES: O=C(O)c1ccc2nc(CC3CCC(c4cccc(OCc5ccc(Cl)cc5F)n4)CC3)n(C[C@@H]3CCO3)c2c1] | (S)-2-((4-(6-((4-chloro-2- fluorobenzyl)oxy)pyridin-2- yl)cyclohexyl)methyl)-1-(oxetan-2- ylmethyl) 1H-benzo[d]imidazole-6- carboxylic acid |
| 104C | 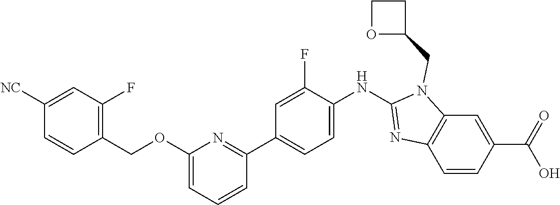 [SMILES: N#Cc1ccc(COc2cccc(-c3ccc(Nc4nc5ccc(C(=O)O)cc5n4C[C@@H]4CCO4)c(F)c3)n2)c(F)c1] | (S)-2-((4-(6-((4-cyano-2- fluorobenzyl)oxy)pyridin-2-yl)-2- fluorophenyl)amino)-1-(oxetan-2- ylmethyl)-1H-benzo[d]imidazole-6- carboxylic acid |
| 105C | 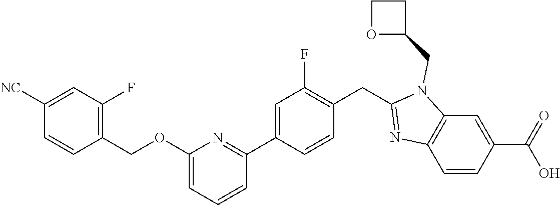 [SMILES: N#Cc1ccc(COc2cccc(-c3ccc(Cc4nc5ccc(C(=O)O)cc5n4C[C@@H]4CCO4)c(F)c3)n2)c(F)c1] | (S)-2-(4-(6-((4-cyano-2- fluorobenzyl)oxy)pyridin-2-yl)-2- fluorophenoxy)-1-(oxetan-2-ylmethyl)- 1H-benzo[d]imidazole-6-carboxylic acid |
| 106C | 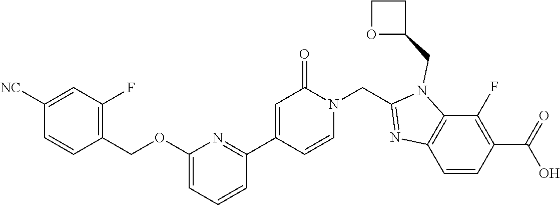 [SMILES: N#Cc1ccc(COc2cccc(-c3ccn(Cc4nc5ccc(C(=O)O)c(F)c5n4C[C@@H]4CCO4)c(=O)c3)n2)c(F)c1] | (S)-2-((6-((4-cyano-2-fluorobenzyl)oxy)- 2′-oxo-[2,4′-bipyridin]-1′(2′H)-yl)methyl)- 7-fluoro-1-(oxetan-2-ylmethyl)-1H- benzo[d]imidazole-6-carboxylic acid |
| 107C | 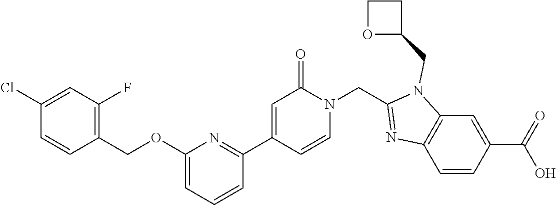 [SMILES: O=C(O)c1ccc2nc(Cn3ccc(-c4cccc(OCc5ccc(Cl)cc5F)n4)cc3=O)n(C[C@@H]3CCO3)c2c1] | (S)-2-((6-((4-chloro-2-fluorobenzyl)oxy)- 2′-oxo-[2,4′-bipyridin]-1′(2′H)-yl)methyl)- 1-(oxetan-2-ylmethyl)-1H- benzo[d]imidazole-6-carboxylic acid |
| 108C | 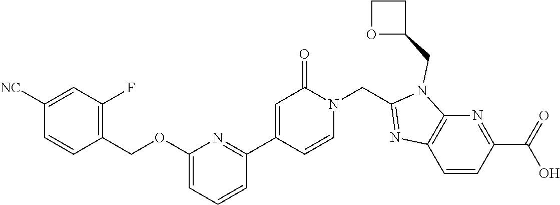 [SMILES: N#Cc1ccc(COc2cccc(-c3ccn(Cc4nc5ccc(C(=O)O)nc5n4C[C@@H]4CCO4)c(=O)c3)n2)c(F)c1] | (S)-2-((6-((4-cyano-2-fluorobenzyl)oxy)- 2′-oxo-[2,4′-bipyridin]-1′(2′H)-yl)methyl)- 3-(oxetan-2-ylmethyl)-3H-imidazo[4,5- b]pyridine-5-carboxylic acid |
| 109C | 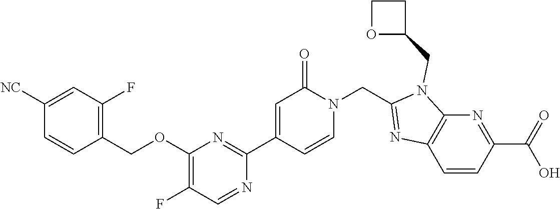 [SMILES: N#Cc1ccc(COc2nc(-c3ccn(Cc4nc5ccc(C(=O)O)nc5n4C[C@@H]4CCO4)c(=O)c3)ncc2F)c(F)c1] | (S)-2-((4-(4-((4-cyano-2- fluorobenzyl)oxy)-5-fluoropyrimidin-2-yl)- 2-oxopyridin-1(2H)-yl)methyl)-3-(oxetan- 2-ylmethyl)-3H-imidazo[4,5-b]pyridine-5- carboxylic acid |
| 110C | 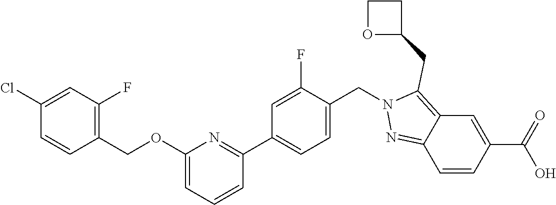 [SMILES: O=C(O)c1ccc2nn(Cc3ccc(-c4cccc(OCc5ccc(Cl)cc5F)n4)cc3F)c(C[C@@H]3CCO3)c2c1] | 2-(4-(6-(4-chloro-2- fluorobenzyloxy)pyridin-2-yl)-2- fluorobenzyl)-3-(oxetan-2-ylmethyl)-2H- indazole-5-carboxylic acid |
| 111C | 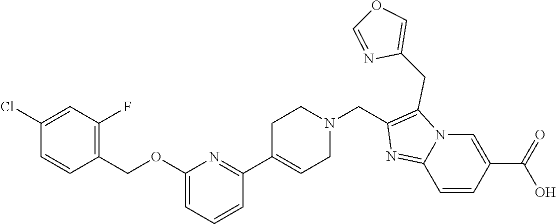 [SMILES: O=C(O)c1ccc2nc(CN3CC=C(c4cccc(OCc5ccc(Cl)cc5F)n4)CC3)c(Cc3cocn3)n2c1] | 2-((6-((4-chloro-2-fluorobenzyl)oxy)-3′,6′- dihydro-[2,4′-bipyridin]-1′(2′H)-yl)methyl)- 3-(oxazol-4-ylmethyl)imidazo[1,2- alpyridine-6-carboxylic acid |
| 112C | 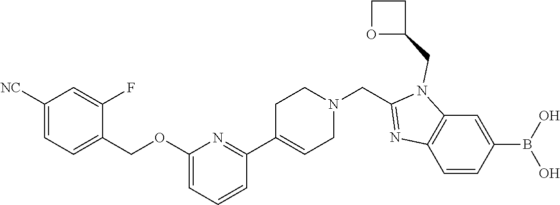 [SMILES: N#Cc1ccc(COc2cccc(C3=CCN(Cc4nc5ccc(B(O)O)cc5n4C[C@@H]4CCO4)CC3)n2)c(F)c1] | (S)-(2-((6-((4-cyano-2-fluorobenzyl)oxy)- 3′,6′-dihydro-[2,4′-bipyridin]-1′(2′H)- yl)methyl)-1-(oxetan-2-ylmethyl)-1H- benzo[d]imidazol-6-yl)boronic acid |
| 113C | 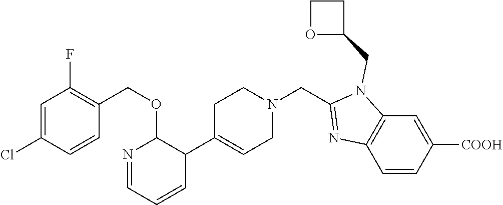 [SMILES: O=C(O)c1ccc2nc(CN3CC=C(C4C=CC=NC4OCc4ccc(Cl)cc4F)CC3)n(C[C@@H]3CCO3)c2c1] | (S)-2-((2-((4-chloro-2-fluorobenzyl)oxy)- 3′,6′-dihydro-[3,4′-bipyridin]-1′(2′H)- yl)methyl)-1-(oxetan-2-ylmethyl)-1H- benzo[d]imidazole-6-carboxylic acid |
| 114C | 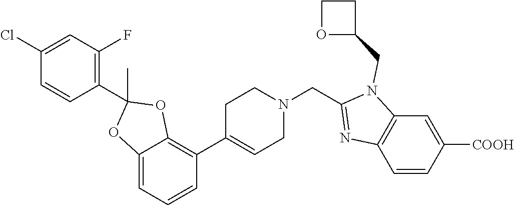 [SMILES: CC1(c2ccc(Cl)cc2F)Oc2cccc(C3=CCN(Cc4nc5ccc(C(=O)O)cc5n4C[C@@H]4CCO4)CC3)c2O1] | 2-((4-(2-(4-chloro-2-fluorophenyl)-2- methylbenzo[d][1,3]dioxol-4-yl)-5,6- dihydropyridin-1(2H)-yl)methyl)-1-((S)- oxetan-2-ylmethyl)-1H- benzo[d]imidazole-6-carboxylic acid |
| 115C | 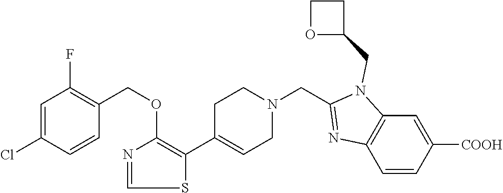 [SMILES: O=C(O)c1ccc2nc(CN3CC=C(c4scnc4OCc4ccc(Cl)cc4F)CC3)n(C[C@@H]3CCO3)c2c1] | (S)-2-((4-(4-((4-chloro-2- fluorobenzyl)oxy)thiazol-5-yl)-3,6- dihydropyridin-1(2H)-yl)methyl)-1- (oxetan-2-ylmethyl)-1H- benzo[d]imidazole-6-carboxylic acid |
| 116C | 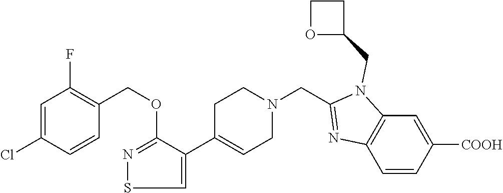 [SMILES: O=C(O)c1ccc2nc(CN3CC=C(c4csnc4OCc4ccc(Cl)cc4F)CC3)n(C[C@@H]3CCO3)c2c1] | (S)-2-((4-(3-(4-chloro-2- fluorobenzyloxy)isothiazol-4-yl)-5,6- dihydropyridin-1(2H)-yl)methyl)-1- (oxetan-2-ylmethyl)-1H- benzo[d]imidazole-6-carboxylic acid |
| 117C | 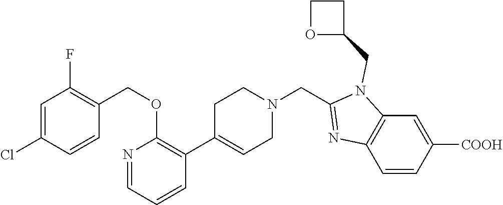 [SMILES: O=C(O)c1ccc2nc(CN3CC=C(c4cccnc4OCc4ccc(Cl)cc4F)CC3)n(C[C@@H]3CCO3)c2c1] | (S)-2-((4-(2-(4-cyano-2- fluorobenzyloxy)pyridin-3-yl)-5,6- dihydropyridin-1(2H)-yl)methyl)-1- (oxetan-2-ylmethyl)-1H- benzo[d]imidazole-6-carboxylic acid |
| 118C | 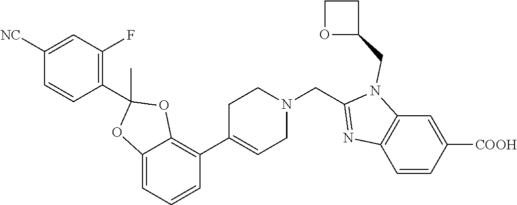 [SMILES: CC1(c2ccc(C#N)cc2F)Oc2cccc(C3=CCN(Cc4nc5ccc(C(=O)O)cc5n4C[C@@H]4CCO4)CC3)c2O1] | 2-((4-(2-(4-cyano-2-fluorophenyl)-2- methylbenzo[d][1,3]dioxol-4-yl)-3,6- dihydropyridin-1(2H)-yl)methyl)-1-(((S)- oxetan-2-yl)methyl)-1H- benzo[d]imidazole-6-carboxylic acid |

In one embodiment, the GLP-1R agonist is a compound of Formula E-I:

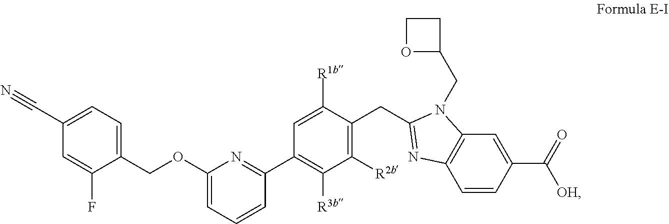

or a pharmaceutically acceptable salt thereof,
wherein:
   R^{1b″} is H or F
   R^{2b″} is H or F; and
   R^{3b″} is H or CH₃.

In one embodiment, the GLP-1R agonist is a compound of Formula E-Ia or C-Ib:

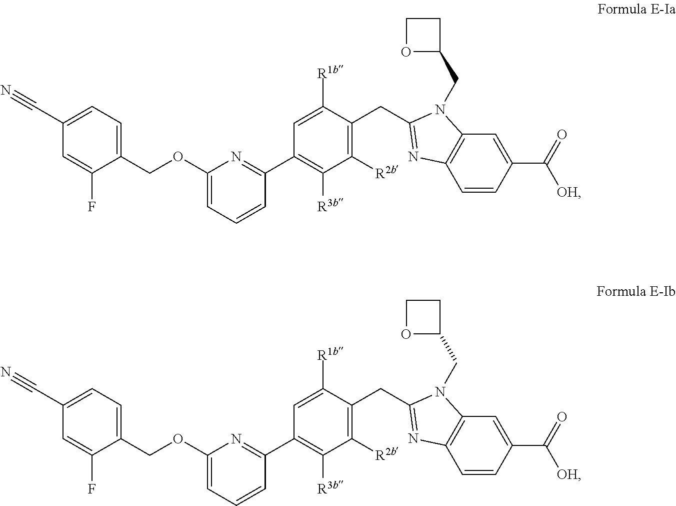

or a pharmaceutically acceptable salt thereof.

In one embodiment, the GLP-1R agonist is a compound of Formula E-II:

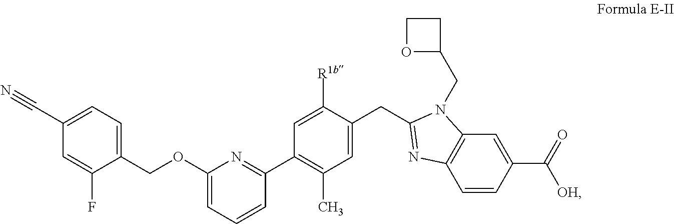

or a pharmaceutically acceptable salt thereof.

In one embodiment, the GLP-1R agonist is a compound of Formula E-IIa or E-IIb:

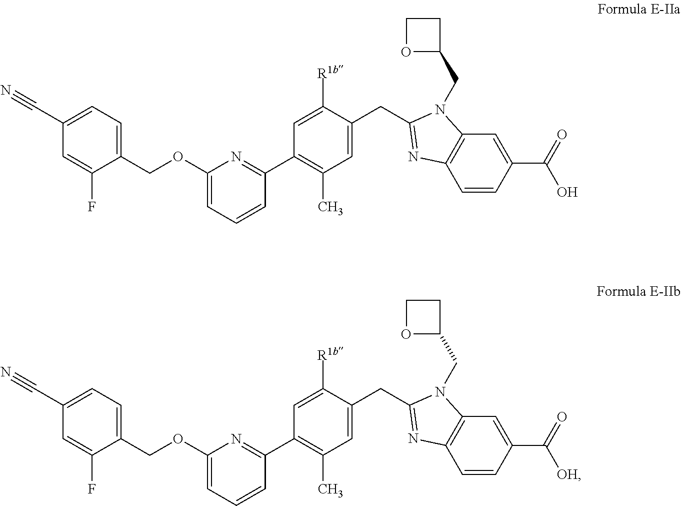

or a pharmaceutically acceptable salt thereof, wherein R^{1″} is H or F.

In one embodiment, the GLP-1R agonist is a compound of Formula E-III:

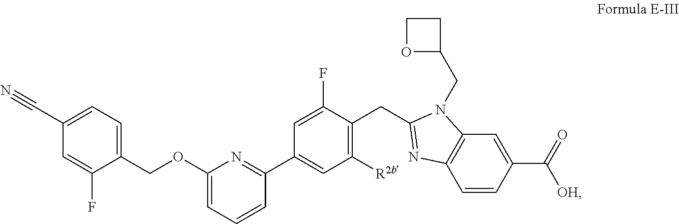

or a pharmaceutically acceptable salt thereof, wherein R^{2b″} is H or F.

In one embodiment, the GLP-1R agonist is a compound of Formula E-IIIa or E-IIIb:

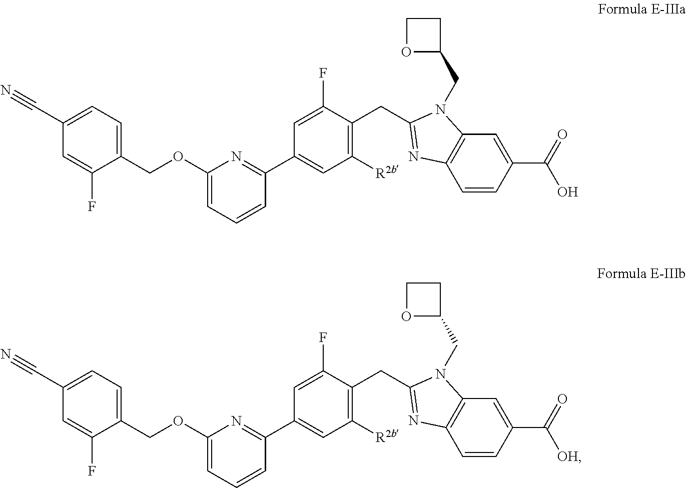

or a pharmaceutically acceptable salt thereof.

The GLP-1R agonist compounds of the disclosure are compounds comprising a molecular weight of from about 400 Da to about 1000 Da, from about 400 Da to about 500 Da, from about 500 Da to about 600 Da, from about 600 Da to about 1000 Da, from about 450 Da to about 550 Da, from about 400 Da to about 450 Da, from about 450 Da to about 500 Da, from about 500 Da to about 550 Da, from about 550 Da to about 600 Da, from about 600 Da to about 650 Da, from about 650 Da to about 700 Da, from about 700 Da to about 750 Da, from about 750 Da to about 800 Da, from about 800 Da to about 850 Da, from about 850 Da to about 900 Da, from about 900 Da to about 950 Da, and from about 950 Da to about 1000 Da. In some embodiments, the GLP-1R agonist compounds of the disclosure comprise a molecular weight of from about 450 Da to about 500 Da. In some embodiments, the GLP-1R agonist compounds of the disclosure comprise a molecular weight of from about 500 Da to about 550 Da. In some embodiments, the GLP-1R agonist compounds of the disclosure comprise a molecular weight of from about 550 Da to about 600 Da. In some embodiments, the GLP-1R agonist compounds of the disclosure comprise a molecular weight of from about 600 Da to about 1000 Da.

TABLE 4 describes additional GLP-1R agonist compounds that can be used in combination with the GIPR antagonist compounds described herein.

TABLE 4
Additional GLP-1R agonist compounds for use in GIPR antagonist combination therapy

| GLP1R | | |
|---|---|---|
| Ag | Structure | IUPAC |
| 119C | 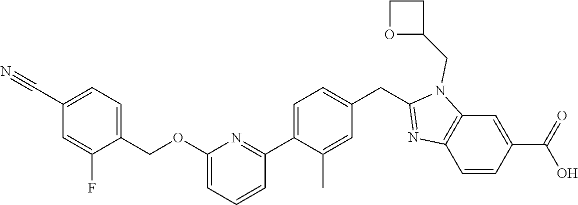 [SMILES: Cc1cc(Cc2nc3ccc(C(=O)O)cc3n2CC2CCO2)ccc1-c1cccc(OCc2ccc(C#N)cc2F)n1] | 2-[[4-[6-[(4-cyano-2-fluoro- phenyl)methoxy]-2- pyridyl]-3-methyl- phenyl]methyl]-3-[oxetan-2- yl]methyl]benzimidazole- 5-carboxylic acid |
| 120C | 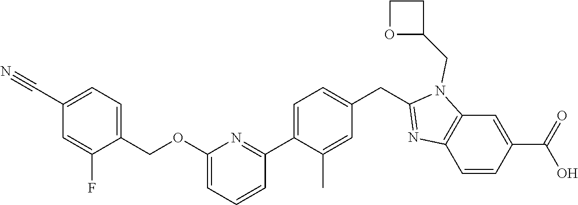 [SMILES: Cc1cc(Cc2nc3ccc(C(=O)O)cc3n2CC2CCO2)ccc1-c1cccc(OCc2ccc(C#N)cc2F)n1] | 2-[[4-[6-[(4-cyano-2-fluoro- phenyl)methoxy]-2- pyridyl]-3-methyl- phenyl]methyl]-3- [(2S)-oxetan-2- yl]methyl]benzimidazole- 5-carboxylic acid |
| 121C | 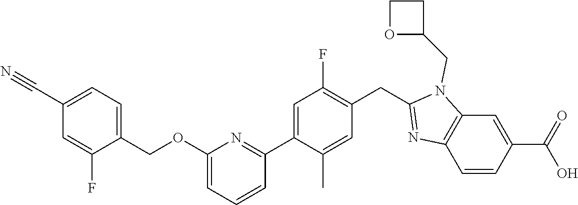 [SMILES: Cc1cc(Cc2nc3ccc(C(=O)O)cc3n2CC2CCO2)c(F)cc1-c1cccc(OCc2ccc(C#N)cc2F)n1] | 2-[4-[6-[(4-cyano-2-fluoro- phenyl)methoxy]-2- pyridyl]-2-fluoro-5- methyl-phenyl]methyl]- 3-[oxetan-2- yl]methyl]benzimidazole- 5-carboxylic acid |
| 122C | 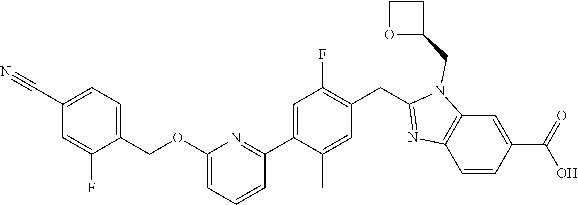 [SMILES: Cc1cc(Cc2nc3ccc(C(=O)O)cc3n2C[C@@H]2CCO2)c(F)cc1-c1cccc(OCc2ccc(C#N)cc2F)n1] | 2-[4-[6-[(4-cyano-2-fluoro- phenyl)methoxy]-2- pyridyl]-2-fluoro-5- methyl-phenyl]methyl]- 3-[(2S)-oxetan-2- yl]methyl]benzimidazole- 5-carboxylic acid |
| 123C | 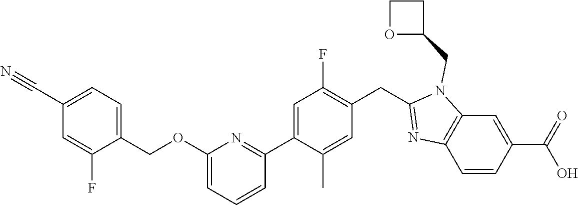 [SMILES: Cc1cc(Cc2nc3ccc(C(=O)O)cc3n2C[C@@H]2CCO2)c(F)cc1-c1cccc(OCc2ccc(C#N)cc2F)n1] | tert-butylammonium; 2-[4-[6-[(4-cyano- 2-fluoro-phenyl)methoxy]- 2-pyridyl]-2- fluoro-5-methyl-phenyl] methyl]-3-[(2S)- oxetan-2-yl]methyl] benzimidazole-5- carboxylate |
| 124C | 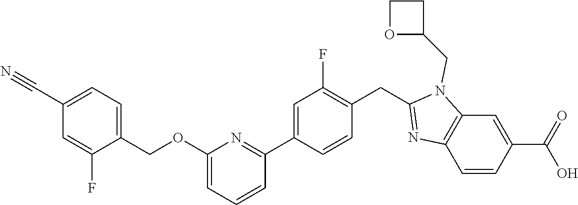 [SMILES: N#Cc1ccc(COc2cccc(-c3ccc(Cc4nc5ccc(C(=O)O)cc5n4CC4CCO4)c(F)c3)n2)c(F)c1] | 2-[[4-[6-[(4-Cyano-2-fluoro- phenyl)methoxy]-2- pyridyl]-2-fluoro- phenyl]methyl]-3-[oxetan-2- ylmethyl]benzimidazole- 5-carboxylic acid |
| 125C | 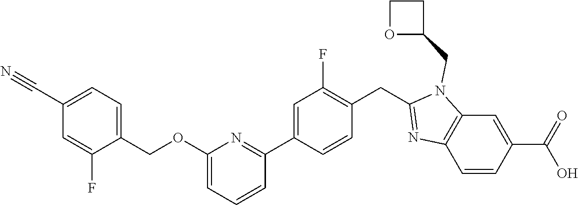 [SMILES: N#Cc1ccc(COc2cccc(-c3ccc(Cc4nc5ccc(C(=O)O)cc5n4C[C@@H]4CCO4)c(F)c3)n2)c(F)c1] | 2-[4-[6-[(4-Cyano-2-fluoro- phenyl)methoxy]- 2-pyridyl]-2-fluoro- phenyl]methyl]-3-[(2S)-oxetan-2- ylmethyl]benzimidazole- 5-carboxylic acid |
| 126C | 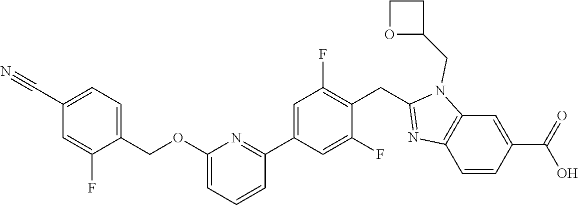 [SMILES: N#Cc1ccc(COc2cccc(-c3cc(F)c(Cc4nc5ccc(C(=O)O)cc5n4CC4CCO4)c(F)c3)n2)c(F)c1] | 2-[[4-[6-[(4-Cyano-2-fluoro- phenyl)methoxy]-2- pyridyl]-2,6-difluoro- phenyl]methyl]-3-[oxetan-2- ylmethyl]benzimidazole- 5-carboxylic acid |
| 127C | 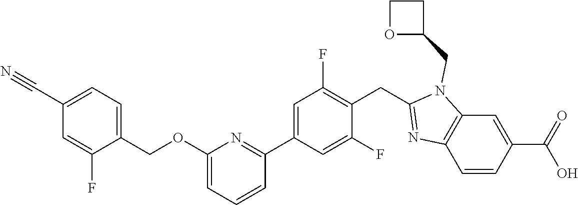 [SMILES: N#Cc1ccc(COc2cccc(-c3cc(F)c(Cc4nc5ccc(C(=O)O)cc5n4C[C@@H]4CCO4)c(F)c3)n2)c(F)c1] | 2-[4-[6-[(4-Cyano-2-fluoro- phenyl)methoxy]-2- pyridyl]-2,6-difluoro- phenyl]methyl]-3-[(2S)-oxetan-2- ylmethyl]benzimidazole- 5-carboxylic acid |
| 128C | 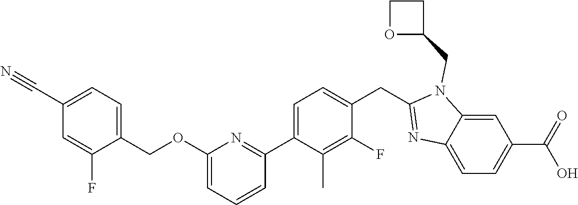 [SMILES: Cc1c(-c2cccc(OCc3ccc(C#N)cc3F)n2)ccc(Cc2nc3ccc(C(=O)O)cc3n2C[C@@H]2CCO2)c1F] | 2-[4-[6-[(4-Cyano-2-fluoro- phenyl)methoxy]-2- pyridyl]-2-fluoro-3- methyl-phenyl]methyl]-3- [(2S)-oxetan-2- yl]methyl]benzimidazole- 5-carboxylic acid |
| 129C | 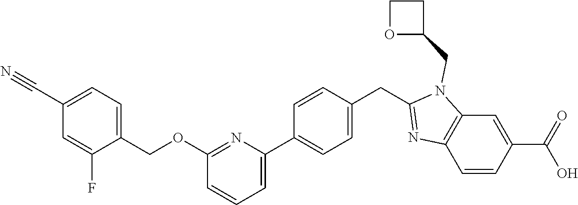 [SMILES: N#Cc1ccc(COc2cccc(-c3ccc(Cc4nc5ccc(C(=O)O)cc5n4C[C@@H]4CCO4)cc3)n2)c(F)c1] | 2-[[4-[6-[(4-Cyano-2-fluoro- phenyl)methoxy]-2- pyridyl]phenyl]methyl]- 3-[(2S)-oxetan- 2-yl]methyl]benzimidazole- 5-carboxylic acid |

In one embodiment, the GLP-1R agonist is a compound of the Formula:

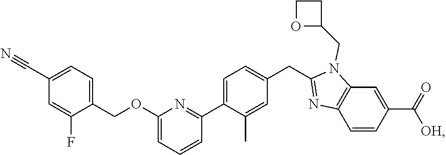 [SMILES: Cc1cc(Cc2nc3ccc(C(=O)O)cc3n2CC2CCO2)ccc1-c1cccc(OCc2ccc(C#N)cc2F)n1]

or a pharmaceutically acceptable salt thereof.

In one embodiment, the GLP-1R agonist is a compound of the Formula:

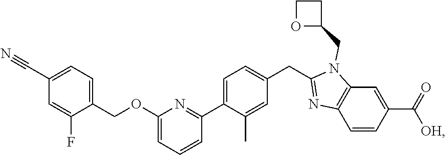 [SMILES: Cc1cc(Cc2nc3ccc(C(=O)O)cc3n2C[C@@H]2CCO2)ccc1-c1cccc(OCc2ccc(C#N)cc2F)n1]

or a pharmaceutically acceptable salt thereof.

In one embodiment, the GLP-1R agonist is a compound of the Formula:

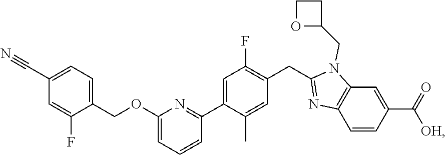 [SMILES: Cc1cc(Cc2nc3ccc(C(=O)O)cc3n2CC2CCO2)c(F)cc1-c1cccc(OCc2ccc(C#N)cc2F)n1]

or a pharmaceutically acceptable salt thereof.

In one embodiment, the GLP-1R agonist is a compound of the Formula:

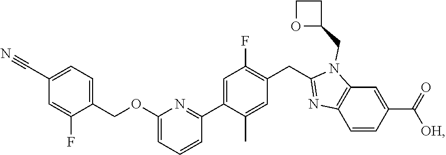 [SMILES: Cc1cc(Cc2nc3ccc(C(=O)O)cc3n2C[C@@H]2CCO2)c(F)cc1-c1cccc(OCc2ccc(C#N)cc2F)n1]

or a pharmaceutically acceptable salt thereof.

In one embodiment, the GLP-1R agonist is a compound of the Formula:

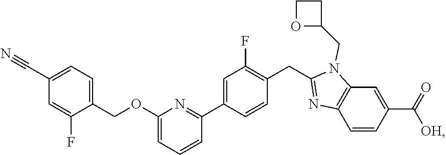 [SMILES: N#Cc1ccc(COc2cccc(-c3ccc(Cc4nc5ccc(C(=O)O)cc5n4CC4CCO4)c(F)c3)n2)c(F)c1]

or a pharmaceutically acceptable salt thereof.

In one embodiment, the GLP-1R agonist is a compound of the Formula:

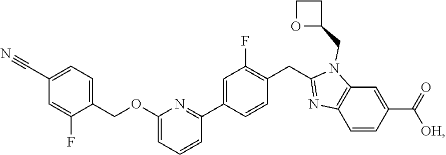 [SMILES: N#Cc1ccc(COc2cccc(-c3ccc(Cc4nc5ccc(C(=O)O)cc5n4C[C@@H]4CCO4)c(F)c3)n2)c(F)c1]

or a pharmaceutically acceptable salt thereof.

In one embodiment, the GLP-1R agonist is a compound of the Formula:

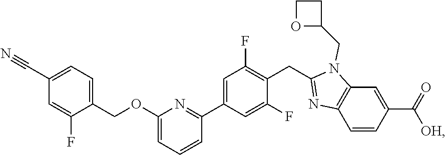 [SMILES: N#Cc1ccc(COc2cccc(-c3cc(F)c(Cc4nc5ccc(C(=O)O)cc5n4CC4CCO4)c(F)c3)n2)c(F)c1]

or a pharmaceutically acceptable salt thereof.

In one embodiment, the GLP-1R agonist is a compound of the Formula:

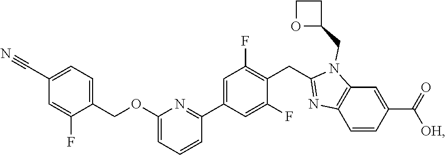 [SMILES: N#Cc1ccc(COc2cccc(-c3cc(F)c(Cc4nc5ccc(C(=O)O)cc5n4C[C@@H]4CCO4)c(F)c3)n2)c(F)c1]

or a pharmaceutically acceptable salt thereof.

In some embodiments, the GLP-1R agonist compound has a structure selected from the group consisting of:

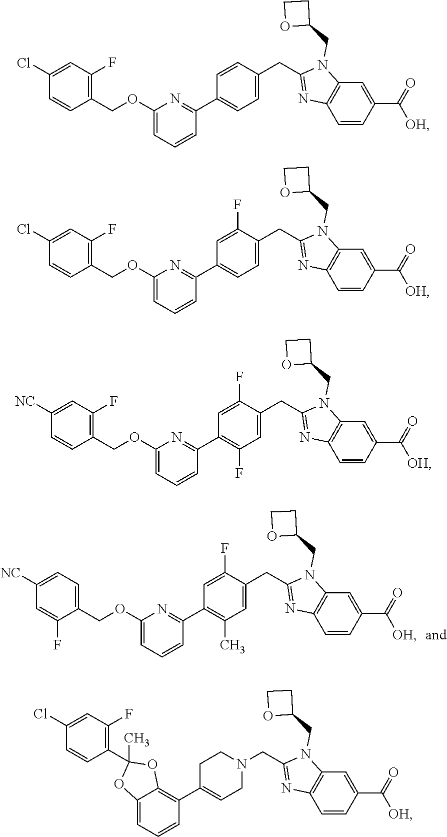 [SMILES: O=C(O)c1ccc2nc(Cc3ccc(-c4cccc(OCc5ccc(Cl)cc5F)n4)cc3)n(C[C@@H]3CCO3)c2c1 ; O=C(O)c1ccc2nc(Cc3ccc(-c4cccc(OCc5ccc(Cl)cc5F)n4)cc3F)n(C[C@@H]3CCO3)c2c1 ; N#Cc1ccc(COc2cccc(-c3cc(F)c(Cc4nc5ccc(C(=O)O)cc5n4C[C@@H]4CCO4)cc3F)n2)c(F)c1 ; Cc1cc(Cc2nc3ccc(C(=O)O)cc3n2C[C@@H]2CCO2)c(F)cc1-c1cccc(OCc2ccc(C#N)cc2F)n1 ; CC1(c2ccc(Cl)cc2F)Oc2cccc(C3=CCN(Cc4nc5ccc(C(=O)O)cc5n4C[C@@H]4CCO4)CC3)c2O1]

or a pharmaceutically acceptable salt thereof.

In some embodiments, the GLP-1R agonist is a compound of the structure

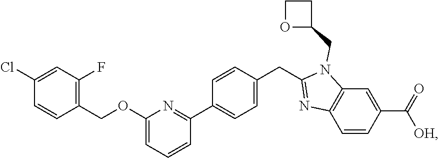 [SMILES: O=C(O)c1ccc2nc(Cc3ccc(-c4cccc(OCc5ccc(Cl)cc5F)n4)cc3)n(C[C@@H]3CCO3)c2c1]

or a pharmaceutically acceptable salt thereof.

In some embodiments, the GLP-1R agonist is a compound of the structure

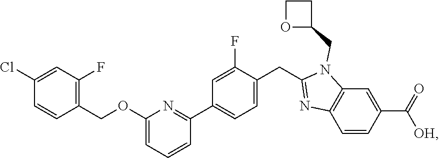 [SMILES: O=C(O)c1ccc2nc(Cc3ccc(-c4cccc(OCc5ccc(Cl)cc5F)n4)cc3F)n(C[C@@H]3CCO3)c2c1]

or a pharmaceutically acceptable salt thereof.

In some embodiments, the GLP-1R agonist is a compound of the structure

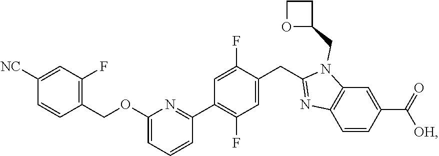 [SMILES: N#Cc1ccc(COc2cccc(-c3cc(F)c(Cc4nc5ccc(C(=O)O)cc5n4C[C@@H]4CCO4)cc3F)n2)c(F)c1]

or a pharmaceutically acceptable salt thereof.

In some embodiments, the GLP-1R agonist is a compound of the structure

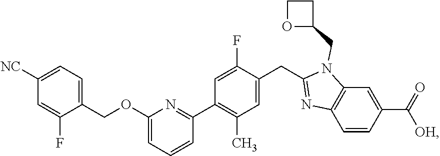 [SMILES: Cc1cc(Cc2nc3ccc(C(=O)O)cc3n2C[C@@H]2CCO2)c(F)cc1-c1cccc(OCc2ccc(C#N)cc2F)n1]

or a pharmaceutically acceptable salt thereof.

In some embodiments, the GLP-1R agonist is a compound of the structure

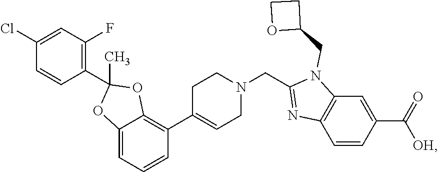 [SMILES: CC1(c2ccc(Cl)cc2F)Oc2cccc(C3=CCN(Cc4nc5ccc(C(=O)O)cc5n4C[C@@H]4CCO4)CC3)c2O1]

or a pharmaceutically acceptable salt thereof.

In one embodiment, the GLP-1 modulating compound is a compound disclosed in WO2023057429A1, WO2023057427A1, WO2023057414A1, WO2023111145A1, or WO2023111144A1.

In one embodiment, the GLP-1 modulating compound is a compound disclosed in US2022409598A1, U.S. Pat. No. 11,643,403B2, US2023002348A1, or WO2023182869A1. In one embodiment, the GLP-1 modulating compound is ID-110521156, or a pharmaceutically acceptable salt thereof. In one embodiment, the compound is:

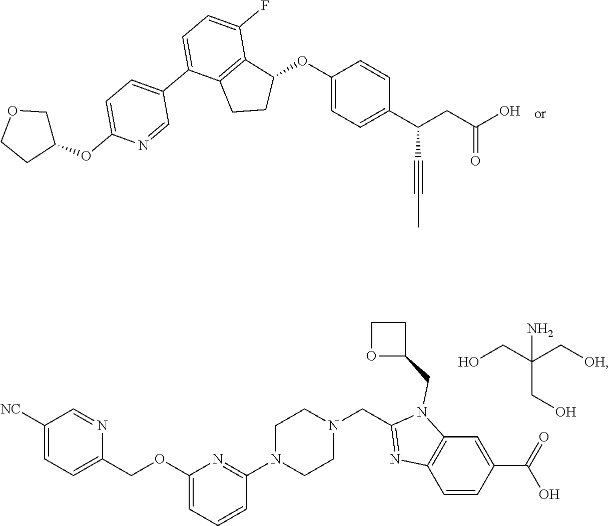

or a pharmaceutically acceptable salt thereof.

In one embodiment, the GLP-1 modulating compound is a compound disclosed in WO2020197926A1, US2022226298A1, U.S. Pat. No. 11,512,065B2, US2023041621A1, US20220289772A1, WO2022216709A1, or WO2023250323A1. In one embodiment, the GLP-1 modulating compound is K-757, or a pharmaceutically acceptable salt thereof. In one embodiment, the GLP-1 modulating compound is K-833, or a pharmaceutically acceptable salt thereof.

In one embodiment, the GLP-1 modulating compound is a compound disclosed in U.S. Pat. No. 11,492,365B2, US2023165846A1, US2023295154A1, U.S. Pat. Nos. 11,897,851B2, 11,926,626B2, US2023322771A1, US2023331732A1, US2023391760A1, WO2023016546A1, WO2023138684A1, WO2023151574A1, WO2023151575A1, WO2023169456A1, WO2023198140A1, or WO2023179542A1. In one embodiment, the GLP-1 modulating compound is GSBR-1290, or a pharmaceutically acceptable salt thereof. In one embodiment, the GLP-1 modulating compound is selected from the group consisting of:

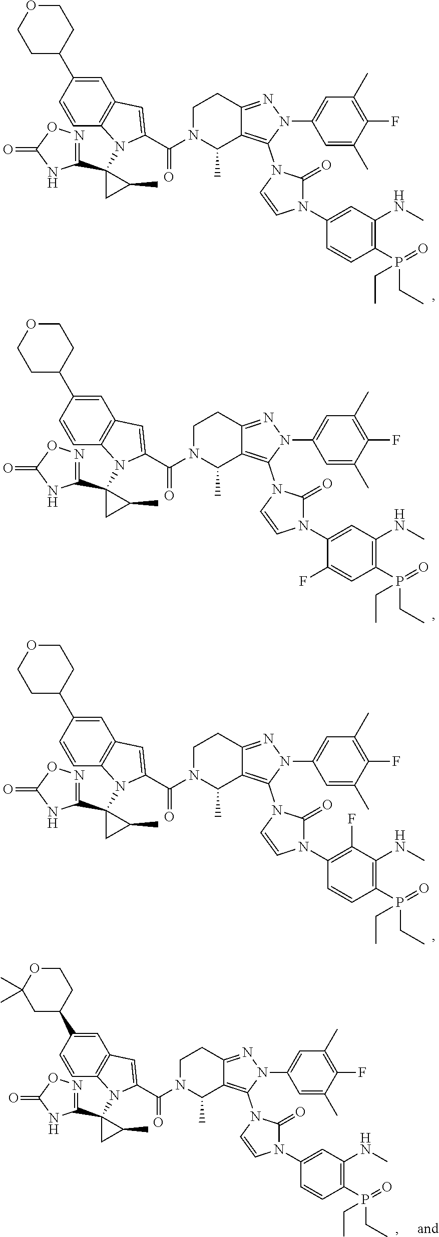

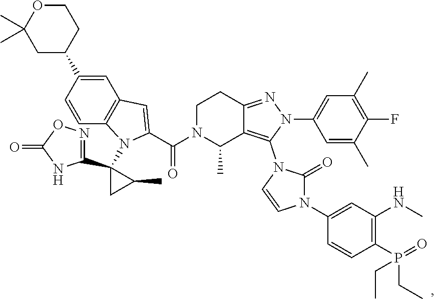 [SMILES: CCP(=O)(CC)c1ccc(-n2ccn(-c3c4c(nn3-c3cc(C)c(F)c(C)c3)CCN(C(=O)c3cc5cc([C@H]6CCOC(C)(C)C6)ccc5n3[C@@]3(c5noc(=O)[nH]5)C[C@@H]3C)[C@H]4C)c2=O)cc1NC]

or a pharmaceutically acceptable salt thereof.

In one embodiment, the GLP-1 modulating compound is

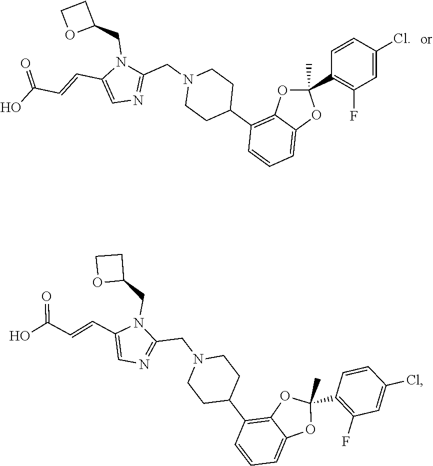 [SMILES: C[C@]1(c2ccc(Cl)cc2F)Oc2cccc(C3CCN(Cc4ncc(/C=C/C(=O)O)n4C[C@@H]4CCO4)CC3)c2O1 ; C[C@@]1(c2ccc(Cl)cc2F)Oc2cccc(C3CCN(Cc4ncc(/C=C/C(=O)O)n4C[C@@H]4CCO4)CC3)c2O1]

or a pharmaceutically acceptable salt thereof.

In one embodiment, the GLP-1 modulating compound is a compound disclosed in U.S. Pat. No. 11,542,272B2. In one embodiment, the GLP-1 modulating compound is XW-014, or a pharmaceutically acceptable salt thereof. In one embodiment, the GLP-1 modulating compound is WXA005, or a pharmaceutically acceptable salt thereof. In one embodiment, the GLP-1 modulating compound is

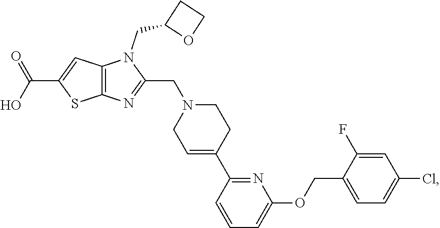 [SMILES: O=C(O)c1cc2c(nc(CN3CC=C(c4cccc(OCc5ccc(Cl)cc5F)n4)CC3)n2C[C@@H]2CCO2)s1]

or a pharmaceutically acceptable salt thereof.

In one embodiment, the GLP-1 modulating compound is a compound disclosed in US2023373927A1, U.S. Pat. No. 8,546,581B2, U.S. Pat. No. 7,947,841B2, WO2020263695A1, U.S. Pat. No. 11,655,242B2, US2023250092A1, or WO2022246019A1. In one embodiment, the GLP-1 modulating compound is LY-3502970, or a pharmaceutically acceptable salt thereof. In one embodiment, the compound is selected from the group consisting of:

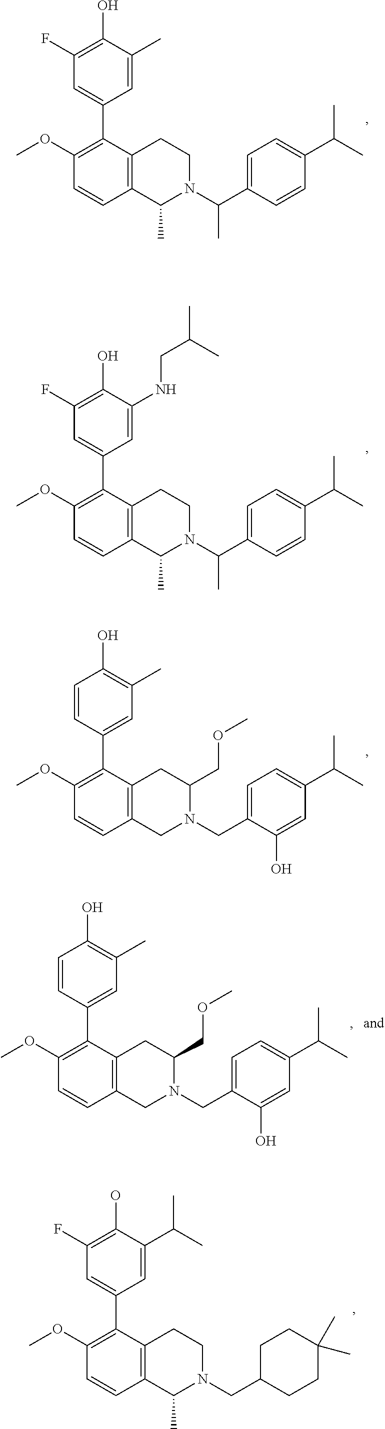

or a pharmaceutically acceptable salt thereof. In one embodiment, the GLP-1 modulating compound is

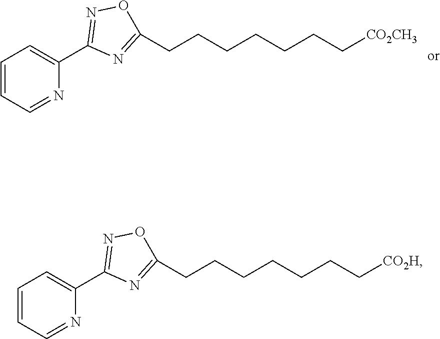 [SMILES: COC(=O)CCCCCCCc1nc(-c2ccccn2)no1 ; O=C(O)CCCCCCCc1nc(-c2ccccn2)no1]

or a pharmaceutically acceptable salt thereof. In one embodiment, the GLP-1 modulating compound is selected from the group consisting of:

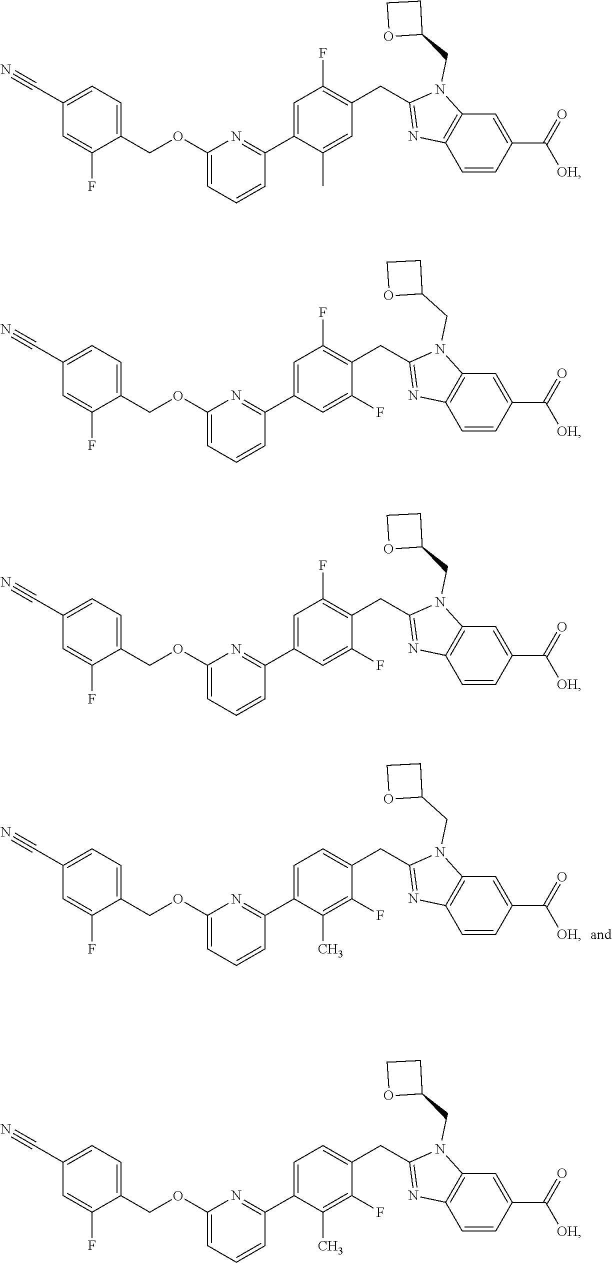 [SMILES: Cc1cc(Cc2nc3ccc(C(=O)O)cc3n2C[C@@H]2CCO2)c(F)cc1-c1cccc(OCc2ccc(C#N)cc2F)n1 ; N#Cc1ccc(COc2cccc(-c3cc(F)c(Cc4nc5ccc(C(=O)O)cc5n4CC4CCO4)c(F)c3)n2)c(F)c1 ; N#Cc1ccc(COc2cccc(-c3cc(F)c(Cc4nc5ccc(C(=O)O)cc5n4C[C@@H]4CCO4)c(F)c3)n2)c(F)c1 ; Cc1c(-c2cccc(OCc3ccc(C#N)cc3F)n2)ccc(Cc2nc3ccc(C(=O)O)cc3n2CC2CCO2)c1F ; Cc1c(-c2cccc(OCc3ccc(C#N)cc3F)n2)ccc(Cc2nc3ccc(C(=O)O)cc3n2C[C@@H]2CCO2)c1F]

or a pharmaceutically acceptable salt thereof.

In one embodiment, the GLP-1 modulating compound is a compound disclosed in WO20220199661A1 or WO2022017338A1. In one embodiment, the GLP-1 modulating compound is ECC5004, or a pharmaceutically acceptable salt thereof. In one embodiment, the GLP-1 modulating compound is selected from the group consisting of:

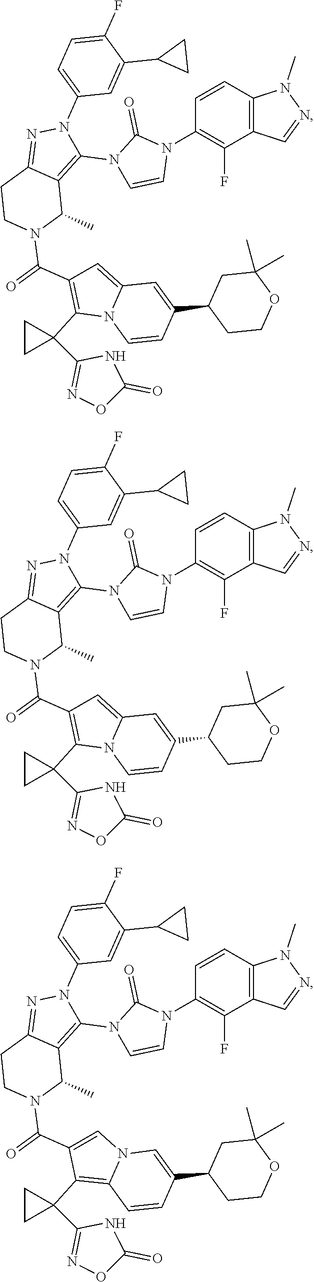

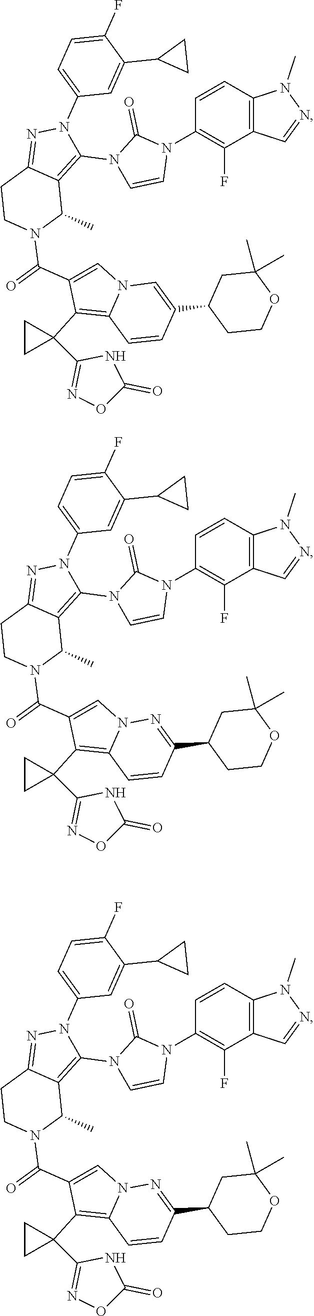

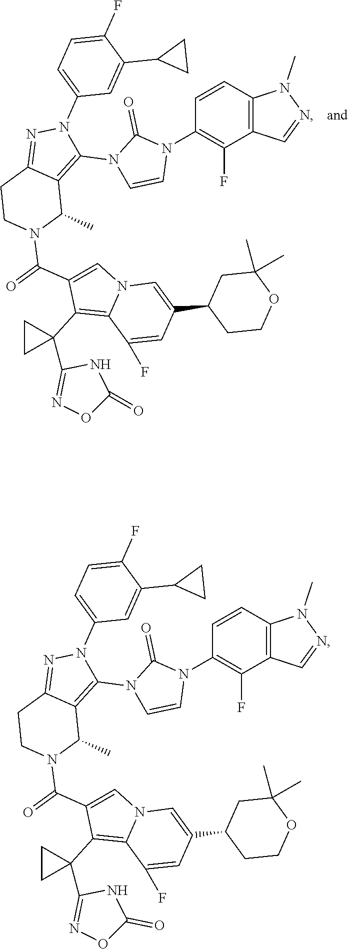 [SMILES: C[C@H]1c2c(nn(-c3ccc(F)c(C4CC4)c3)c2-n2ccn(-c3ccc4c(cnn4C)c3F)c2=O)CCN1C(=O)c1cn2cc([C@@H]3CCOC(C)(C)C3)cc(F)c2c1C1(c2noc(=O)[nH]2)CC1 ; C[C@H]1c2c(nn(-c3ccc(F)c(C4CC4)c3)c2-n2ccn(-c3ccc4c(cnn4C)c3F)c2=O)CCN1C(=O)c1cn2cc([C@H]3CCOC(C)(C)C3)cc(F)c2c1C1(c2noc(=O)[nH]2)CC1]

or a pharmaceutically acceptable salt thereof.

In one embodiment, the GLP-1 modulating compound is a compound disclosed in WO2022235717A1, WO2022165076A1, or WO2024026338A1. In one embodiment, the GLP-1 modulating compound is CT-996, or a pharmaceutically acceptable salt thereof. In one embodiment, the GLP-1 modulating compound is selected from the group consisting of:

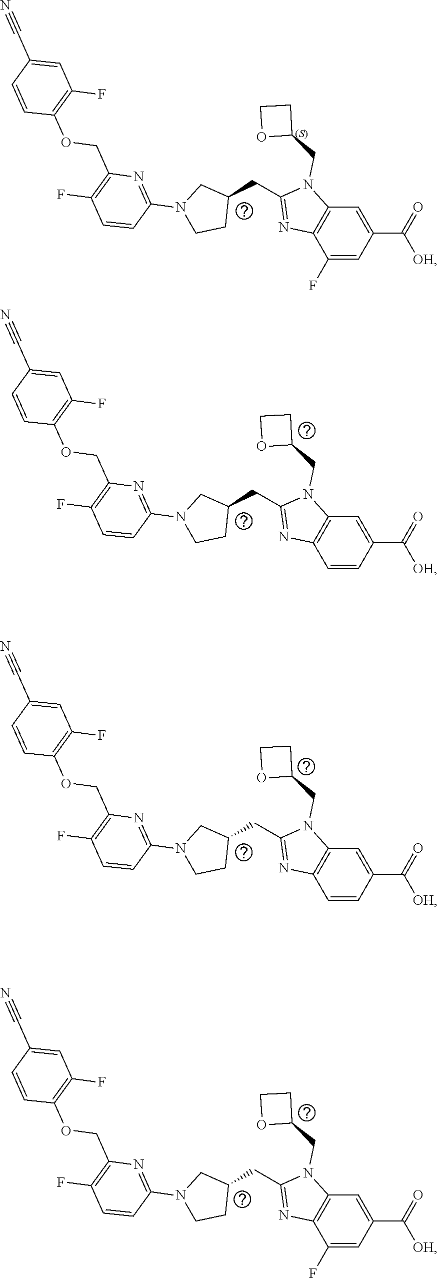

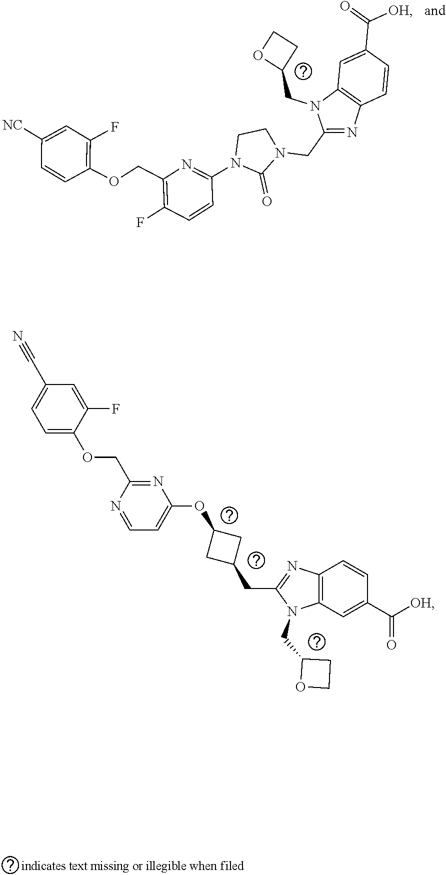

or a pharmaceutically acceptable salt thereof. In one embodiment, the GLP-1 modulating compound is selected from the group consisting of:

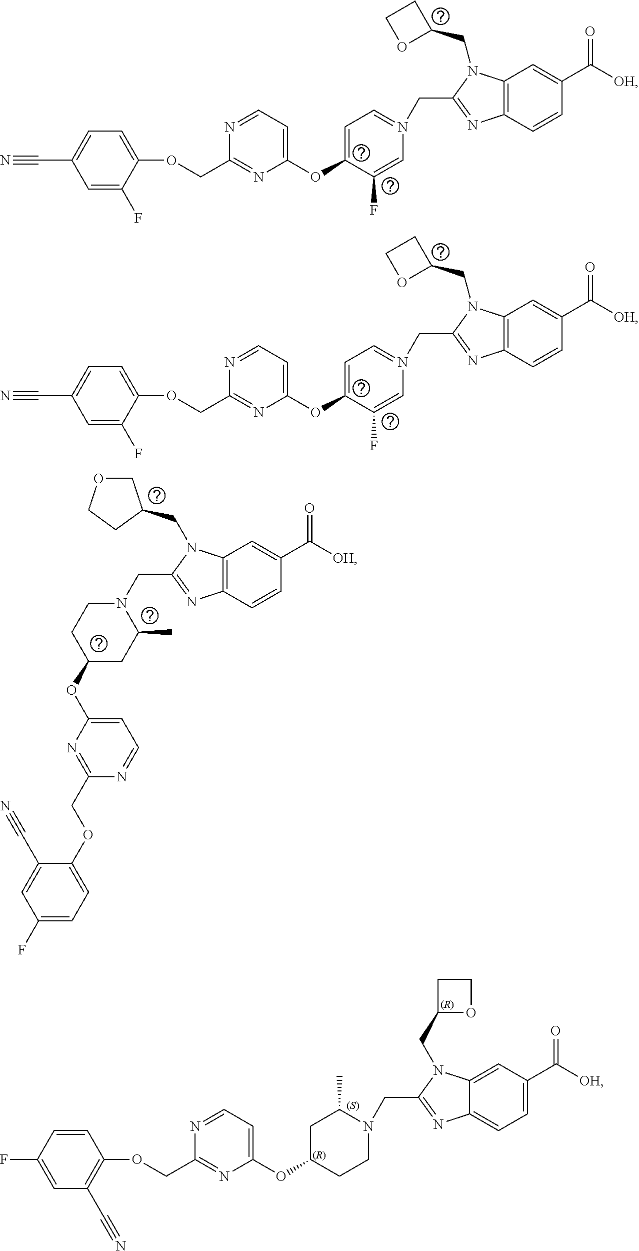

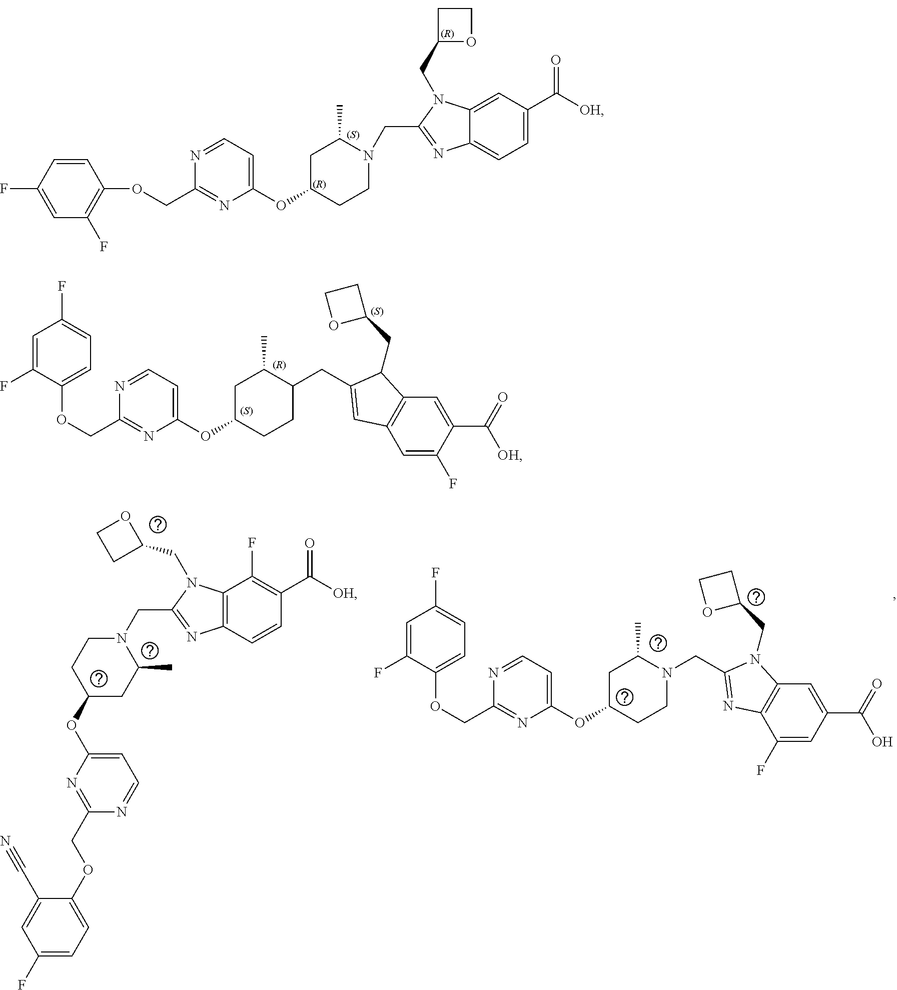 [SMILES: C[C@H]1C[C@@H](Oc2ccnc(COc3ccc(F)cc3F)n2)CCN1Cc1nc2ccc(C(=O)O)cc2n1C[C@H]1CCO1 ; C[C@H]1C[C@@H](Oc2ccnc(COc3ccc(F)cc3F)n2)CCC1CC1=Cc2cc(F)c(C(=O)O)cc2C1C[C@@H]1CCO1 ; C[C@H]1C[C@@H](Oc2ccnc(COc3ccc(F)cc3C#N)n2)CCN1Cc1nc2ccc(C(=O)O)c(F)c2n1C[C@@H]1CCO1 ; C[C@H]1C[C@@H](Oc2ccnc(COc3ccc(F)cc3F)n2)CCN1Cc1nc2c(F)cc(C(=O)O)cc2n1C[C@@H]1CCO1]

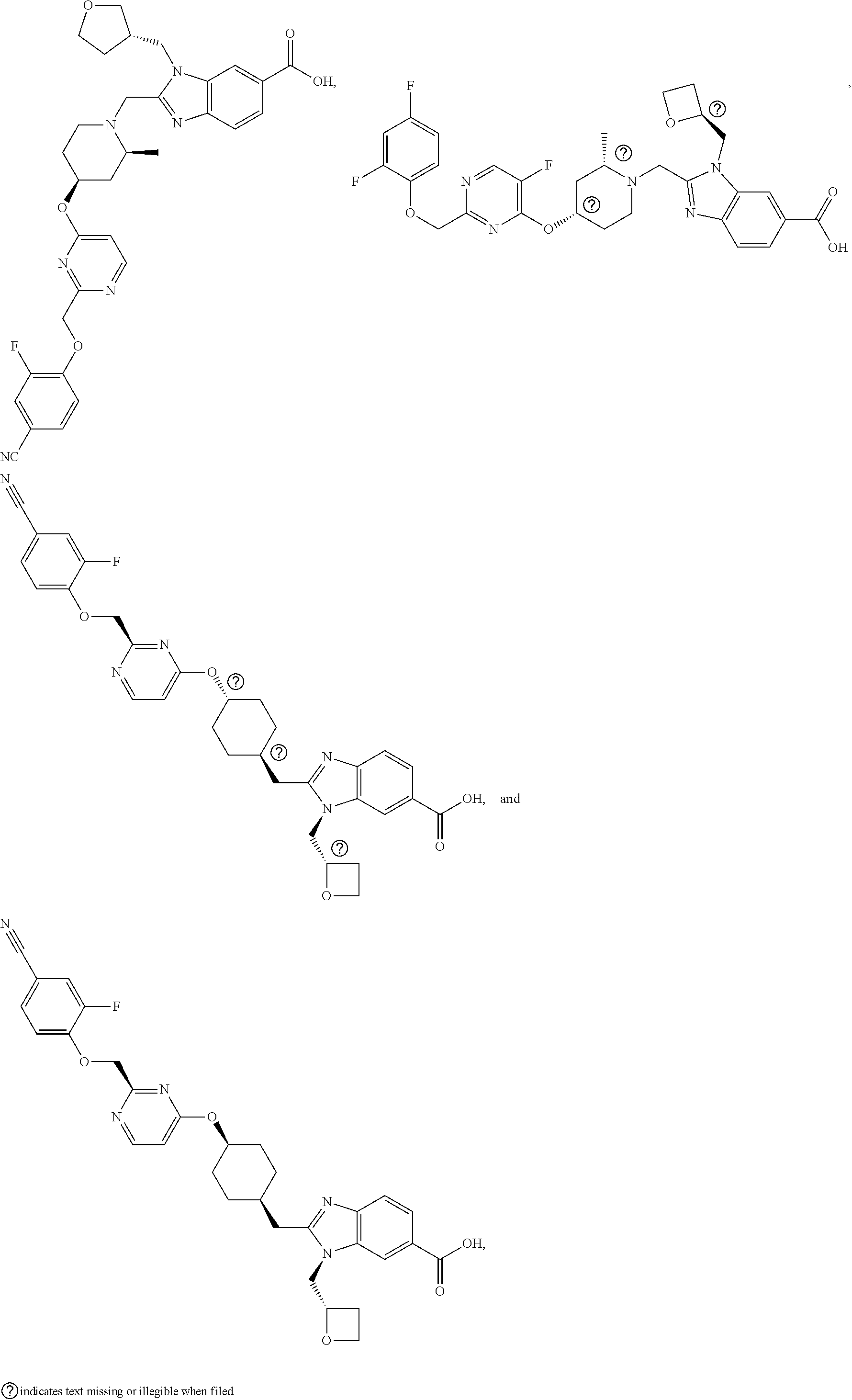

or a pharmaceutically acceptable salt thereof. In one embodiment, the GLP-1 modulating compound is

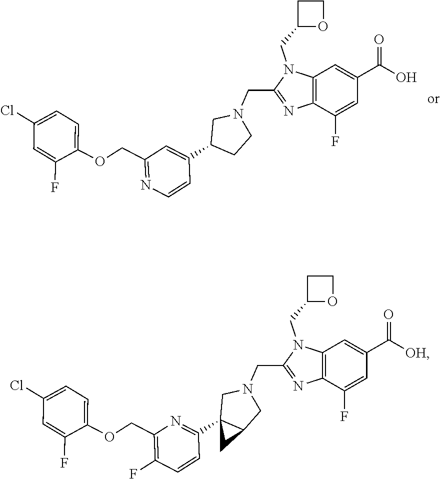 [SMILES: O=C(O)c1cc(F)c2nc(CN3CC[C@H](c4ccnc(COc5ccc(Cl)cc5F)c4)C3)n(C[C@@H]3CCO3)c2c1 ; O=C(O)c1cc(F)c2nc(CN3C[C@H]4C[C@@]4(c4ccc(F)c(COc5ccc(Cl)cc5F)n4)C3)n(C[C@@H]3CCO3)c2c1]

or a pharmaceutically acceptable salt thereof.

In one embodiment, the GLP-1 modulating compound is a compound disclosed in US2023051318A1, WO2016060517A1, or WO2014171762A1. In one embodiment, the GLP-1 modulating compound is HD-7671, or a pharmaceutically acceptable salt thereof.

In one embodiment, the GLP-1 modulating compound is a compound disclosed in CN117069743A or CN101643475A. In one embodiment, the GLP-1 modulating compound is SNG-202, or a pharmaceutically acceptable salt thereof. In one embodiment, the compound is

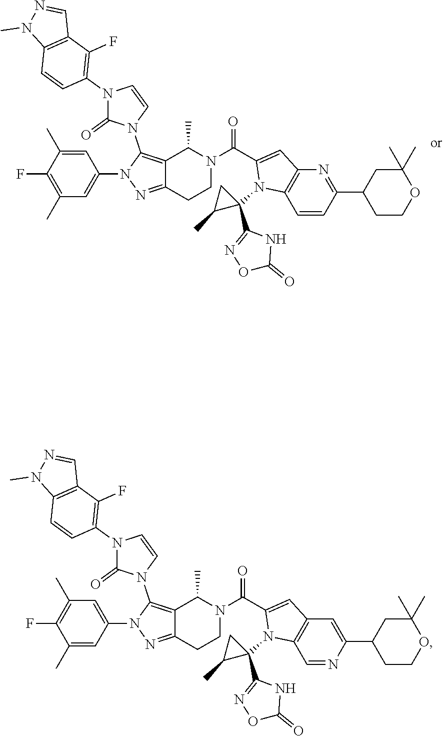

or a pharmaceutically acceptable salt thereof. In one embodiment, the compound is selected from the group consisting of:

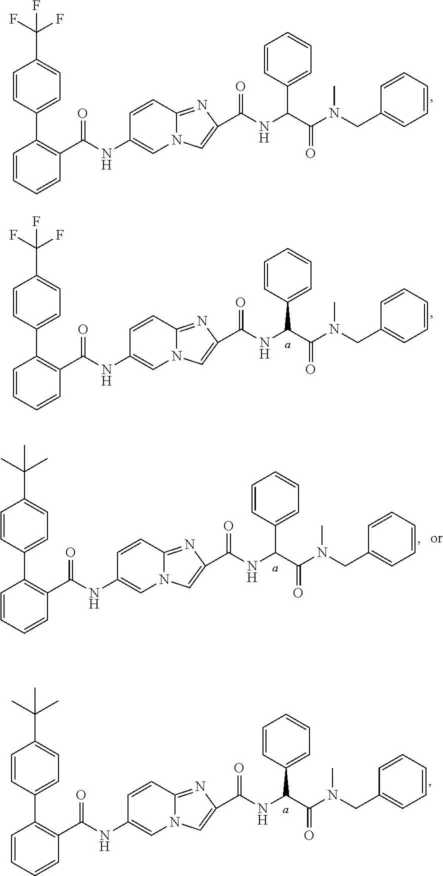

or a pharmaceutically acceptable salt thereof.

In one embodiment, the GLP-1 modulating compound is a compound disclosed in WO2023000834A1, WO2023185821A1, or CN113480534A. In one embodiment, the GLP-1 modulating compound is BEB-808, or a pharmaceutically acceptable salt thereof. In one embodiment, the GLP-1 modulating compound is selected from the group consisting of:

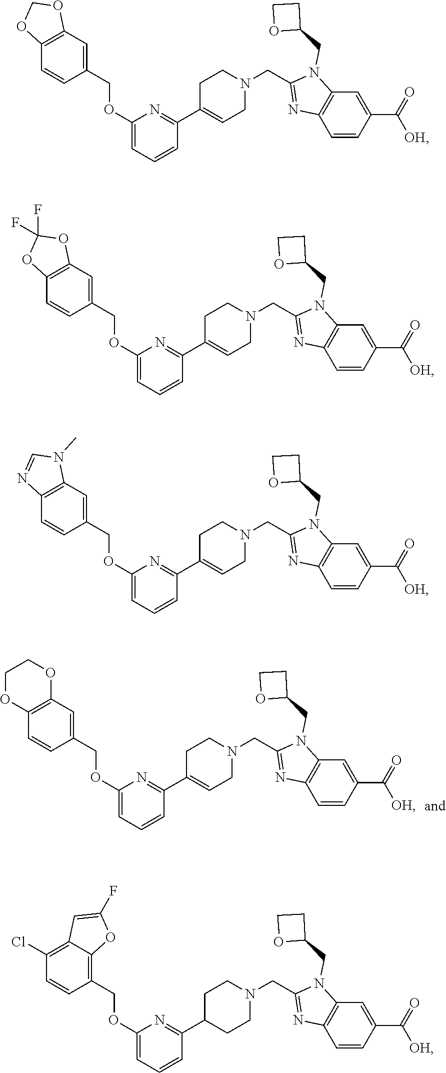 [SMILES: O=C(O)c1ccc2nc(CN3CC=C(c4cccc(OCc5ccc6c(c5)OCO6)n4)CC3)n(C[C@@H]3CCO3)c2c1 ; O=C(O)c1ccc2nc(CN3CC=C(c4cccc(OCc5ccc6c(c5)OC(F)(F)O6)n4)CC3)n(C[C@@H]3CCO3)c2c1 ; Cn1cnc2ccc(COc3cccc(C4=CCN(Cc5nc6ccc(C(=O)O)cc6n5C[C@@H]5CCO5)CC4)n3)cc21 ; O=C(O)c1ccc2nc(CN3CC=C(c4cccc(OCc5ccc6c(c5)OCCO6)n4)CC3)n(C[C@@H]3CCO3)c2c1 ; O=C(O)c1ccc2nc(CN3CCC(c4cccc(OCc5ccc(Cl)c6cc(F)oc56)n4)CC3)n(C[C@@H]3CCO3)c2c1]

or a pharmaceutically acceptable salt thereof.

In one embodiment, the GLP-1 modulating compound is a compound disclosed in WO2011085643A1. In one embodiment, the GLP-1 modulating compound is HSP-004, or a pharmaceutically acceptable salt thereof.

In one embodiment, the GLP-1 modulating compound is a compound disclosed in CN102875567B, WO2010069124A1, CN102241644B, CN101684088B, or CN101684103B. In one embodiment, the GLP-1 modulating compound is TY-705, or a pharmaceutically acceptable salt thereof. In one embodiment, the GLP-1 modulating compound is selected from the group consisting of:

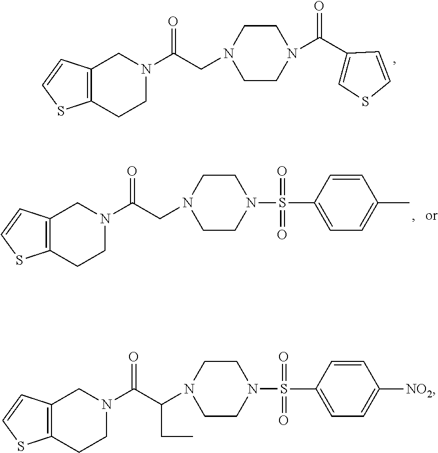 [SMILES: O=C(CN1CCN(C(=O)c2ccsc2)CC1)N1CCc2sccc2C1 ; Cc1ccc(S(=O)(=O)N2CCN(CC(=O)N3CCc4sccc4C3)CC2)cc1 ; CCC(C(=O)N1CCc2sccc2C1)N1CCN(S(=O)(=O)c2ccc([N+](=O)[O-])cc2)CC1]

or a pharmaceutically acceptable salt thereof.

In one embodiment, the GLP-1 modulating compound is a compound disclosed in WO2022061091A1, WO2023051490A1, WO2021242806A1, WO2014113357A1, WO2010114824A1, WO2013142569A1, or WO2009111700A1. In one embodiment, the GLP-1 modulating compound is TTP-273, or a pharmaceutically acceptable salt thereof. In one embodiment, the GLP-1 modulating compound is

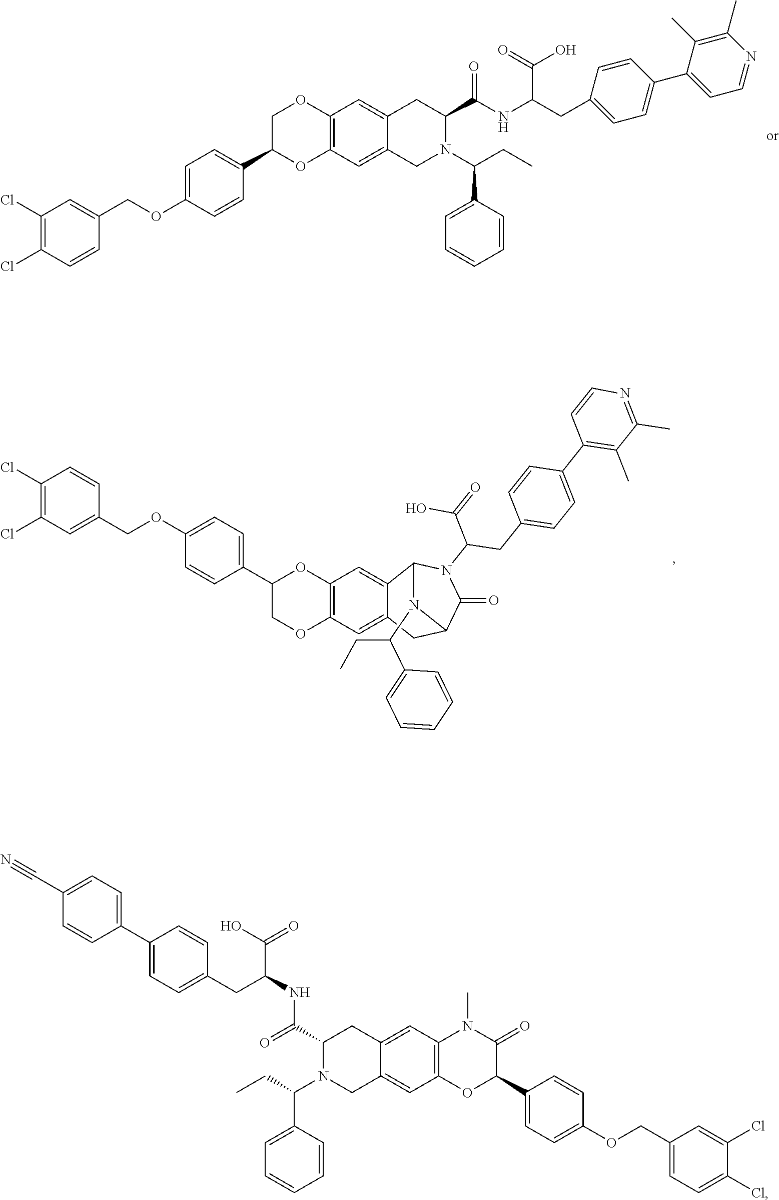 [SMILES: CC[C@@H](c1ccccc1)N1Cc2cc3c(cc2C[C@H]1C(=O)NC(Cc1ccc(-c2ccnc(C)c2C)cc1)C(=O)O)OC[C@H](c1ccc(OCc2ccc(Cl)c(Cl)c2)cc1)O3 ; CCC(c1ccccc1)N1C2Cc3cc4c(cc3C1N(C(Cc1ccc(-c3ccnc(C)c3C)cc1)C(=O)O)C2=O)OC(c1ccc(OCc2ccc(Cl)c(Cl)c2)cc1)CO4 ; CC[C@@H](c1ccccc1)N1Cc2cc3c(cc2C[C@H]1C(=O)N[C@@H](Cc1ccc(-c2ccc(C#N)cc2)cc1)C(=O)O)N(C)C(=O)[C@@H](c1ccc(OCc2ccc(Cl)c(Cl)c2)cc1)O3]

or a pharmaceutically acceptable salt thereof.

In one embodiment, the GLP-1 modulating compound is a compound disclosed in WO2022199458A1, WO2022268152A1, WO2021196949A1, WO2021196951 A1, CN114478497B, WO2022078152A1, CN114907351A, or CN114634510A. In one embodiment, the GLP-1 modulating compound is HDM-1002. In one embodiment, the GLP-1 modulating compound is

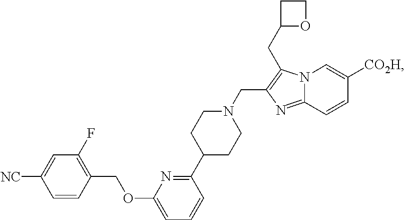 [SMILES: N#Cc1ccc(COc2cccc(C3CCN(Cc4nc5ccc(C(=O)O)cn5c4CC4CCO4)CC3)n2)c(F)c1]

or a pharmaceutically acceptable salt thereof.

In one embodiment, the GLP-1 modulating compound is a compound disclosed in WO2022116693A1. In one embodiment, the GLP-1 modulating compound is VCT-220, or a pharmaceutically acceptable salt thereof. In one embodiment, the GLP-1 modulating compound is

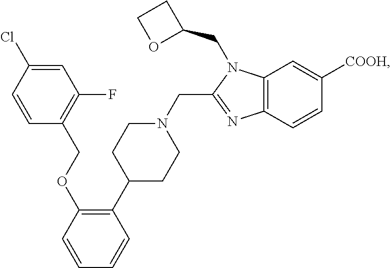 [SMILES: O=C(O)c1ccc2nc(CN3CCC(c4ccccc4OCc4ccc(Cl)cc4F)CC3)n(C[C@@H]3CCO3)c2c1]

or a pharmaceutically acceptable salt thereof.

In one embodiment, the GLP-1 modulating compound is a compound disclosed in WO2022040600A1, WO2023049518A1, WO2023076237A1, or WO2023164050A1. In one embodiment, the GLP-1 modulating compound is TERN-601, or a pharmaceutically acceptable salt thereof.

In one embodiment, the GLP-1 modulating compound is a compound disclosed in WO2023164358A1 or TW202342453A. In one embodiment, the GLP-1 modulating compound is selected from the group consisting of:

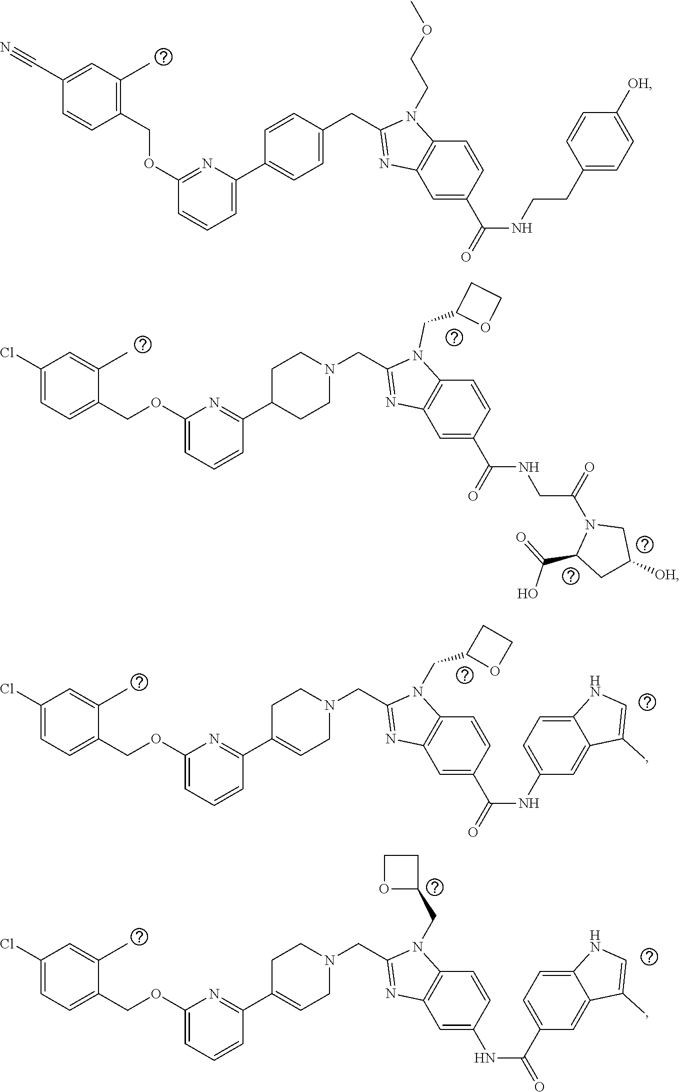

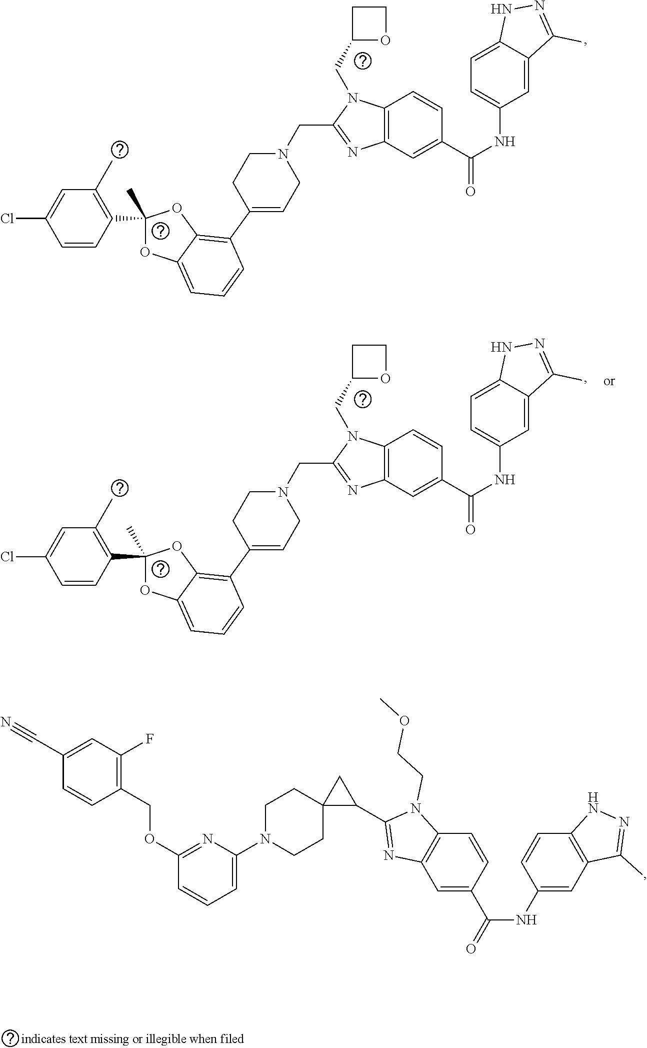

or a pharmaceutically acceptable salt thereof.

In one embodiment, the GLP-1 modulating compound is a compound disclosed in WO2023057427A1, WO2023057429A1, WO2023057414A1, WO2023111144A1, or WO2023111145A1. In one embodiment, the GLP-1 modulating compound is selected from the group consisting of:

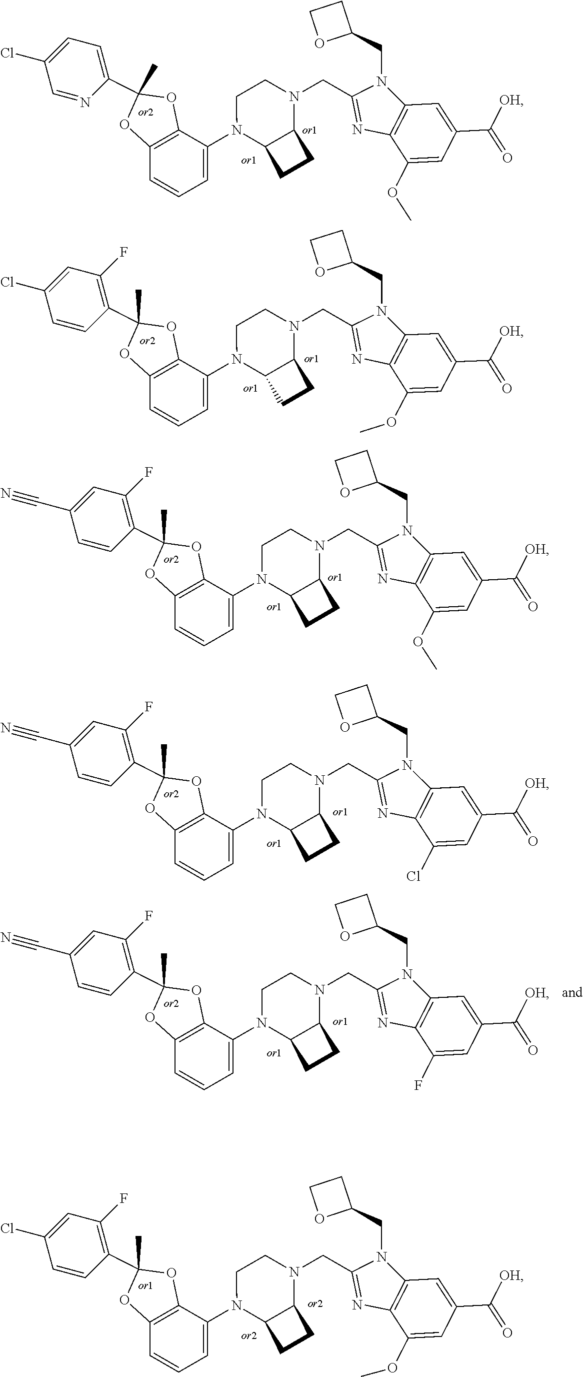

or a pharmaceutically acceptable salt thereof. In one embodiment, the GLP-1 modulating compound is

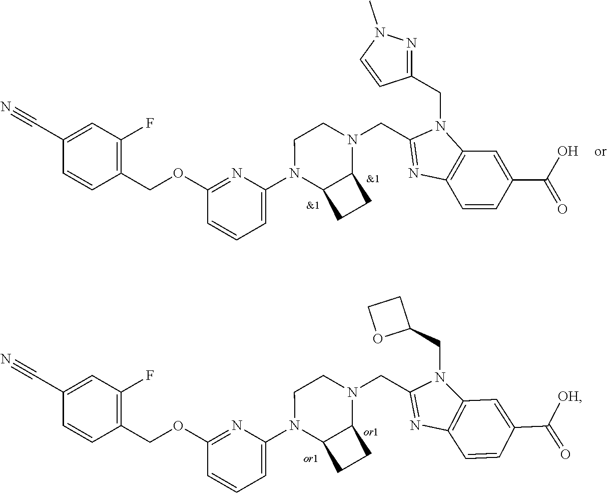 [SMILES: Cn1ccc(Cn2c(CN3CCN(c4cccc(OCc5ccc(C#N)cc5F)n4)[C@@H]4CC[C@@H]43)nc3ccc(C(=O)O)cc32)n1 ; N#Cc1ccc(COc2cccc(N3CCN(Cc4nc5ccc(C(=O)O)cc5n4C[C@@H]4CCO4)[C@H]4CC[C@H]43)n2)c(F)c1]

or a pharmaceutically acceptable salt thereof. In one embodiment, the GLP-1 modulating compound is

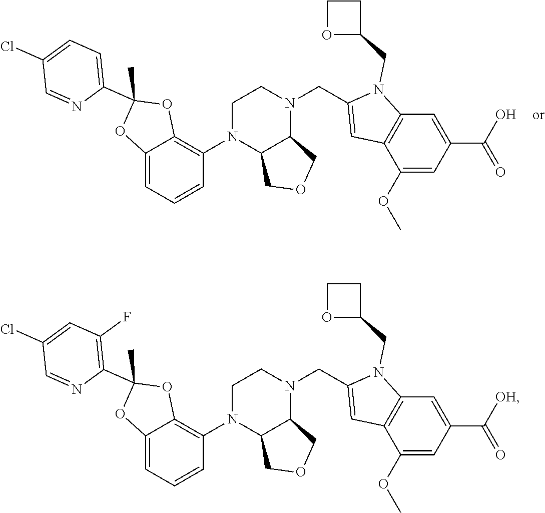 [SMILES: COc1cc(C(=O)O)cc2c1cc(CN1CCN(c3cccc4c3O[C@@](C)(c3ccc(Cl)cn3)O4)[C@@H]3COC[C@@H]31)n2C[C@@H]1CCO1 ; COc1cc(C(=O)O)cc2c1cc(CN1CCN(c3cccc4c3O[C@@](C)(c3ncc(Cl)cc3F)O4)[C@@H]3COC[C@@H]31)n2C[C@@H]1CCO1]

or a pharmaceutically acceptable salt thereof. In one embodiment, the GLP-1 modulating compound is

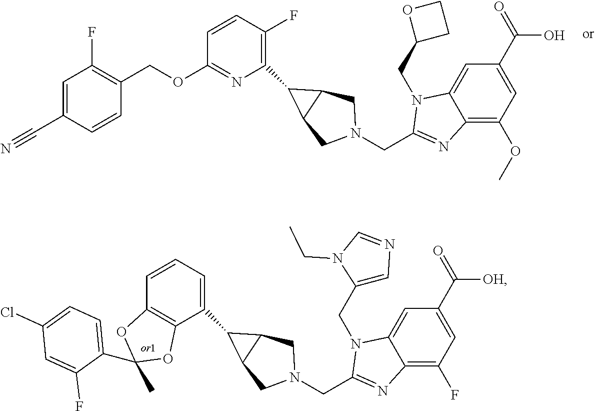 [SMILES: COc1cc(C(=O)O)cc2c1nc(CN1C[C@@H]3[C@H](C1)[C@H]3c1nc(OCc3ccc(C#N)cc3F)ccc1F)n2C[C@@H]1CCO1 ; CCn1cncc1Cn1c(CN2C[C@@H]3[C@H](C2)[C@H]3c2cccc3c2O[C@](C)(c2ccc(Cl)cc2F)O3)nc2c(F)cc(C(=O)O)cc21]

or a pharmaceutically acceptable salt thereof.

In one embodiment, the GLP-1 modulating compound is a compound disclosed in CN117069743A. In one embodiment, the GLP-1 modulating compound is

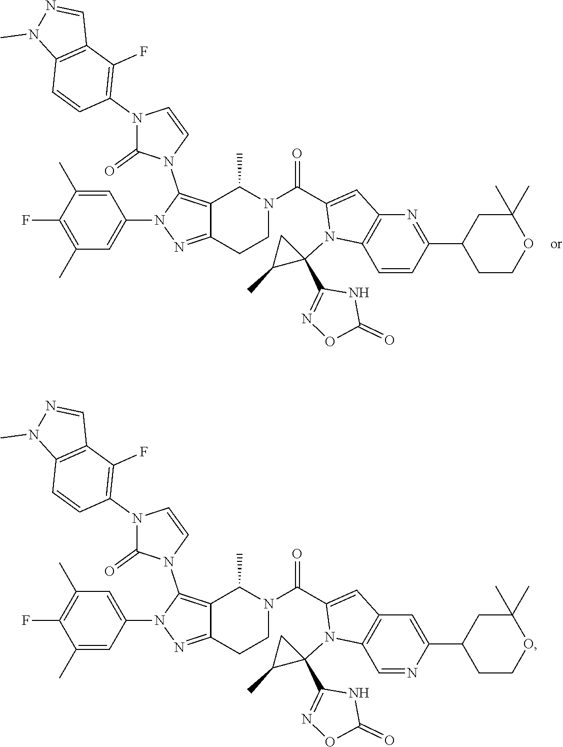

or a pharmaceutically acceptable salt thereof.

In one embodiment, the GLP-1 modulating compound is a compound disclosed in CN116003393A. In one embodiment, the GLP-1 modulating compound is selected from the group consisting of:

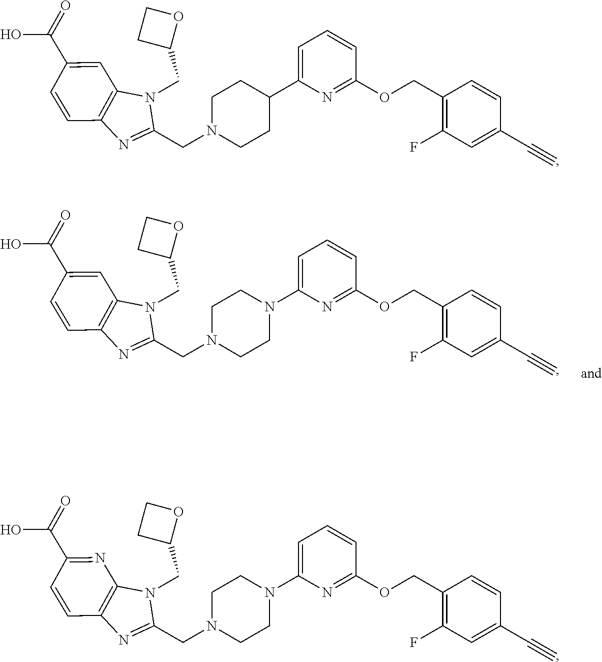

or a pharmaceutically acceptable salt thereof.

In one embodiment, the GLP-1 modulating compound is a compound disclosed in CN113773310B. In one embodiment, the GLP-1 modulating compound is

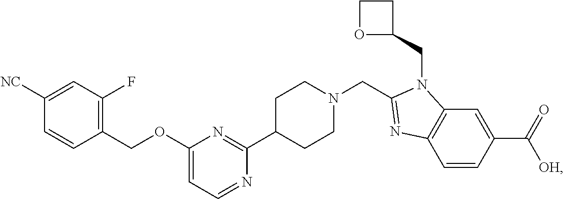 [SMILES: N#Cc1ccc(COc2ccnc(C3CCN(Cc4nc5ccc(C(=O)O)cc5n4C[C@@H]4CCO4)CC3)n2)c(F)c1]

or a pharmaceutically acceptable salt thereof.

In one embodiment, the GLP-1 modulating compound is a compound disclosed in U.S. Pat. No. 11,584,751B1. In one embodiment, the GLP-1 modulating compound is selected from the group consisting of:

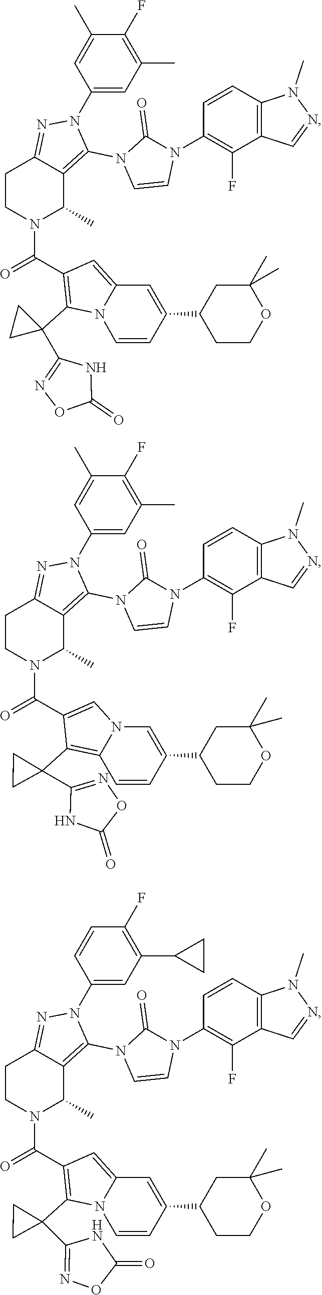

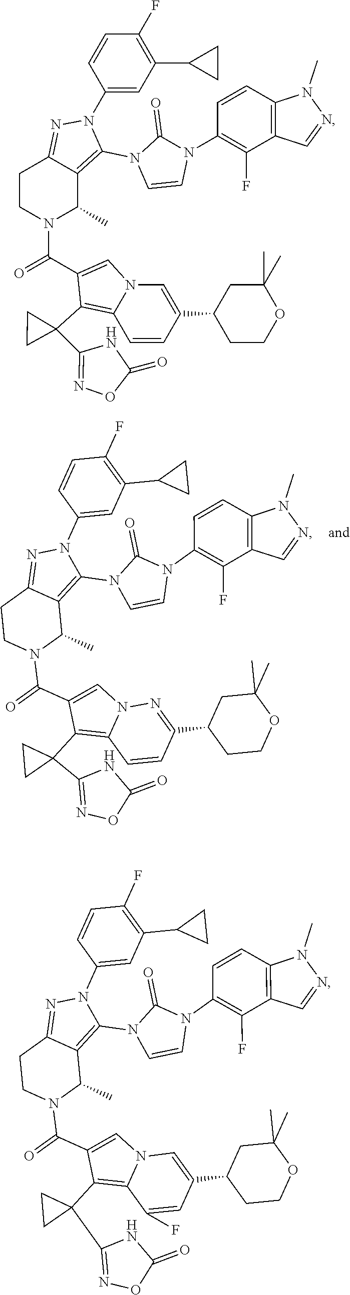

or a pharmaceutically acceptable salt thereof.

In one embodiment, the GLP-1 modulating compound is a compound disclosed in U.S. Ser. No. 10/858,356B32 or US20230382912. In one embodiment, the GLP-1 modulating compound is orforglipron, LY-3502970, or OWL833, or a pharmaceutically acceptable salt thereof. In one embodiment, the GLP-1 modulating compound is orforglipron. In one embodiment, the GLP-1 modulating compound is

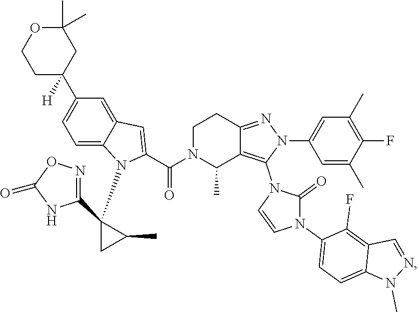

or a pharmaceutically acceptable salt thereof. In one embodiment, the GLP-1 modulating compound is selected from the group consisting of:

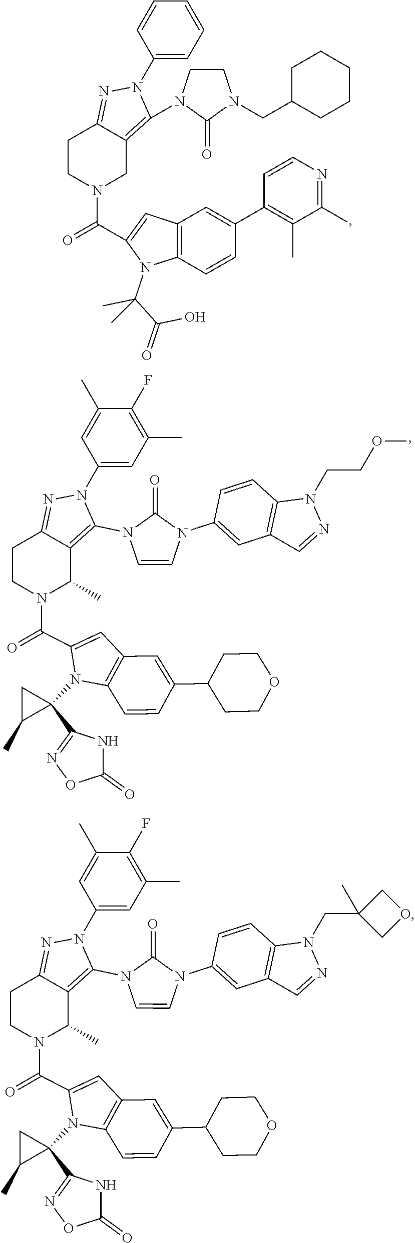

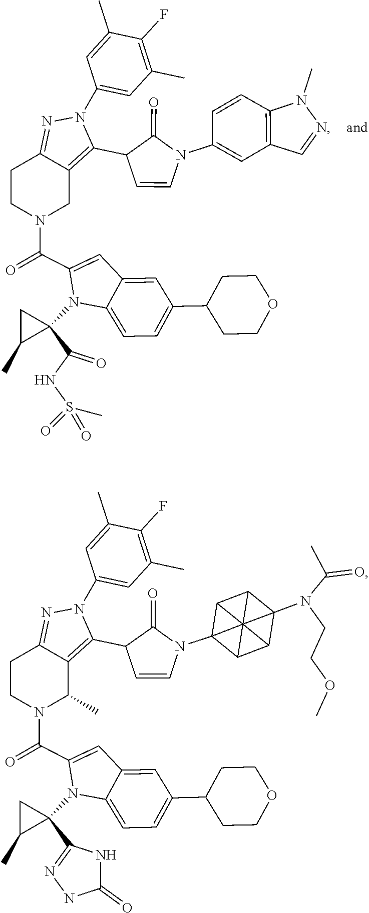

or a pharmaceutically acceptable salt thereof.

In one embodiment, the GLP-1 modulating compound is a compound disclosed in WO2020263695A1 or WO2022246019A1. In one embodiment, the GLP-1 modulating compound is selected from the group consisting of

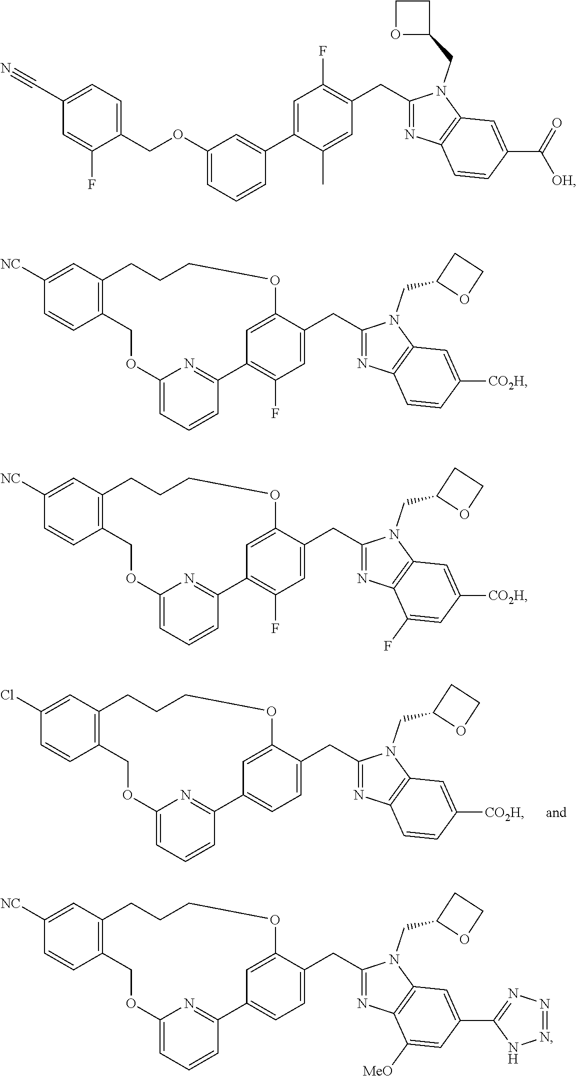

or a pharmaceutically acceptable salt thereof.

In one embodiment, the GLP-1 modulating compound is a compound disclosed in WO2021155841 A1, WO2022052958A1, WO2023016546A1, WO2022048665A1, WO2021219019A1, WO2022042691 A1, WO2022078380A1, WO2023198140A1, WO2023179542A1, WO2023169456A1, WO2023151575A1, WO2023151574A1, US20230192633A1, WO2023138684A1, US20230174565A1, US20230391760A1, US20230331732A1, US20230295154A1, or US20230165846A1. In one embodiment, the GLP-1 modulating compound is selected from the group consisting of:

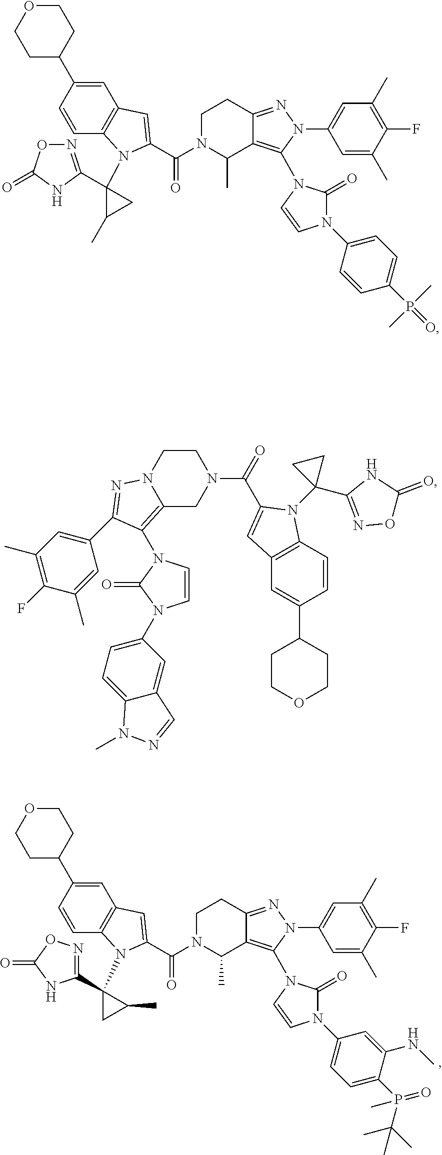 [SMILES: Cc1cc(-n2nc3c(c2-n2ccn(-c4ccc(P(C)(C)=O)cc4)c2=O)C(C)N(C(=O)c2cc4cc(C5CCOCC5)ccc4n2C2(c4noc(=O)[nH]4)CC2C)CC3)cc(C)c1F ; Cc1cc(-c2nn3c(c2-n2ccn(-c4ccc5c(cnn5C)c4)c2=O)CN(C(=O)c2cc4cc(C5CCOCC5)ccc4n2C2(c4noc(=O)[nH]4)CC2)CC3)cc(C)c1F ; CNc1cc(-n2ccn(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)ccc1P(C)(=O)C(C)(C)C]

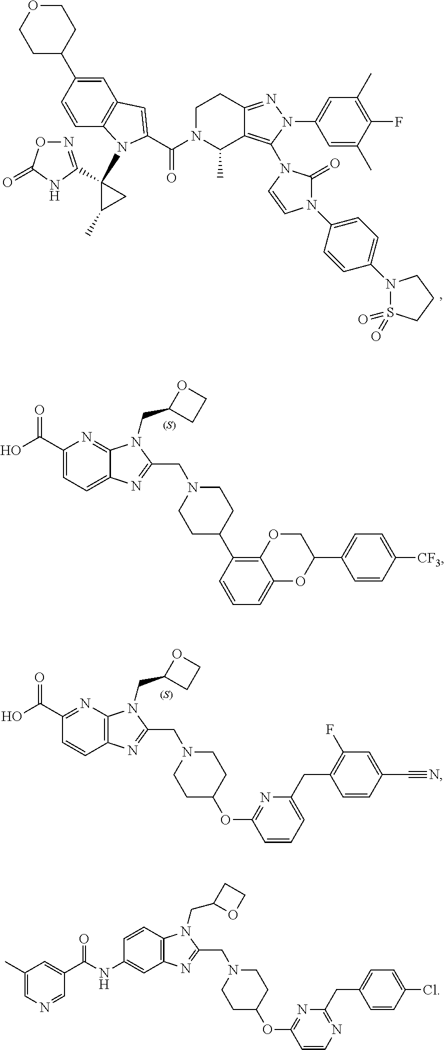

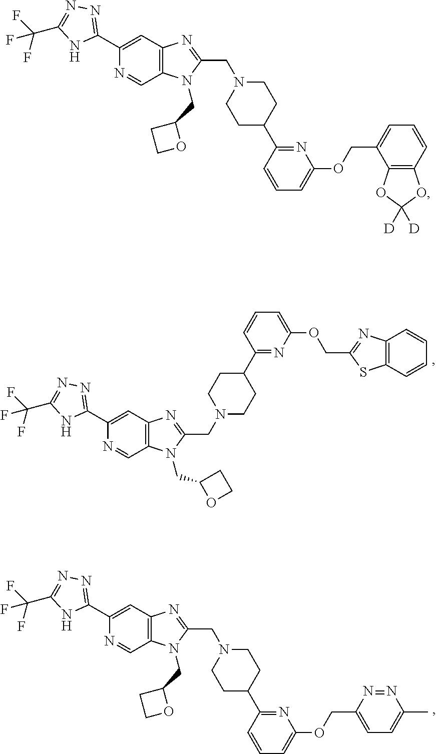 [SMILES: [2H]C1([2H])Oc2cccc(COc3cccc(C4CCN(Cc5nc6cc(-c7nnc(C(F)(F)F)[nH]7)ncc6n5C[C@@H]5CCO5)CC4)n3)c2O1 ; FC(F)(F)c1nnc(-c2cc3nc(CN4CCC(c5cccc(OCc6nc7ccccc7s6)n5)CC4)n(C[C@@H]4CCO4)c3cn2)[nH]1 ; Cc1ccc(COc2cccc(C3CCN(Cc4nc5cc(-c6nnc(C(F)(F)F)[nH]6)ncc5n4C[C@@H]4CCO4)CC3)n2)nn1]

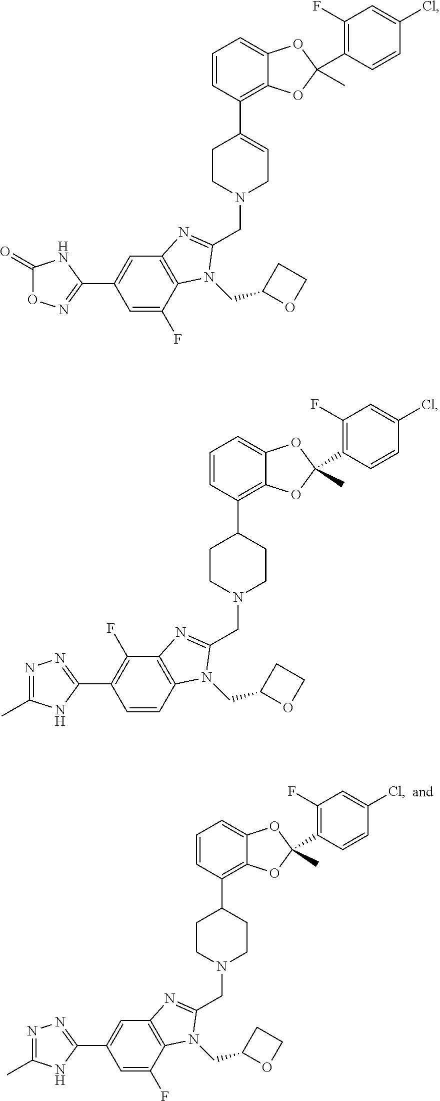 [SMILES: CC1(c2ccc(Cl)cc2F)Oc2cccc(C3=CCN(Cc4nc5cc(-c6noc(=O)[nH]6)cc(F)c5n4C[C@@H]4CCO4)CC3)c2O1 ; Cc1nnc(-c2ccc3c(nc(CN4CCC(c5cccc6c5O[C@@](C)(c5ccc(Cl)cc5F)O6)CC4)n3C[C@@H]3CCO3)c2F)[nH]1 ; Cc1nnc(-c2cc(F)c3c(c2)nc(CN2CCC(c4cccc5c4O[C@@](C)(c4ccc(Cl)cc4F)O5)CC2)n3C[C@@H]2CCO2)[nH]1]

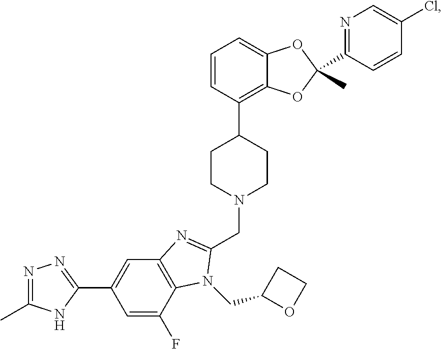 [SMILES: Cc1nnc(-c2cc(F)c3c(c2)nc(CN2CCC(c4cccc5c4O[C@@](C)(c4ccc(Cl)cn4)O5)CC2)n3C[C@@H]2CCO2)[nH]1]

or a pharmaceutically acceptable salt thereof. In one embodiment, the GLP-1 modulating compound is selected from the group consisting of

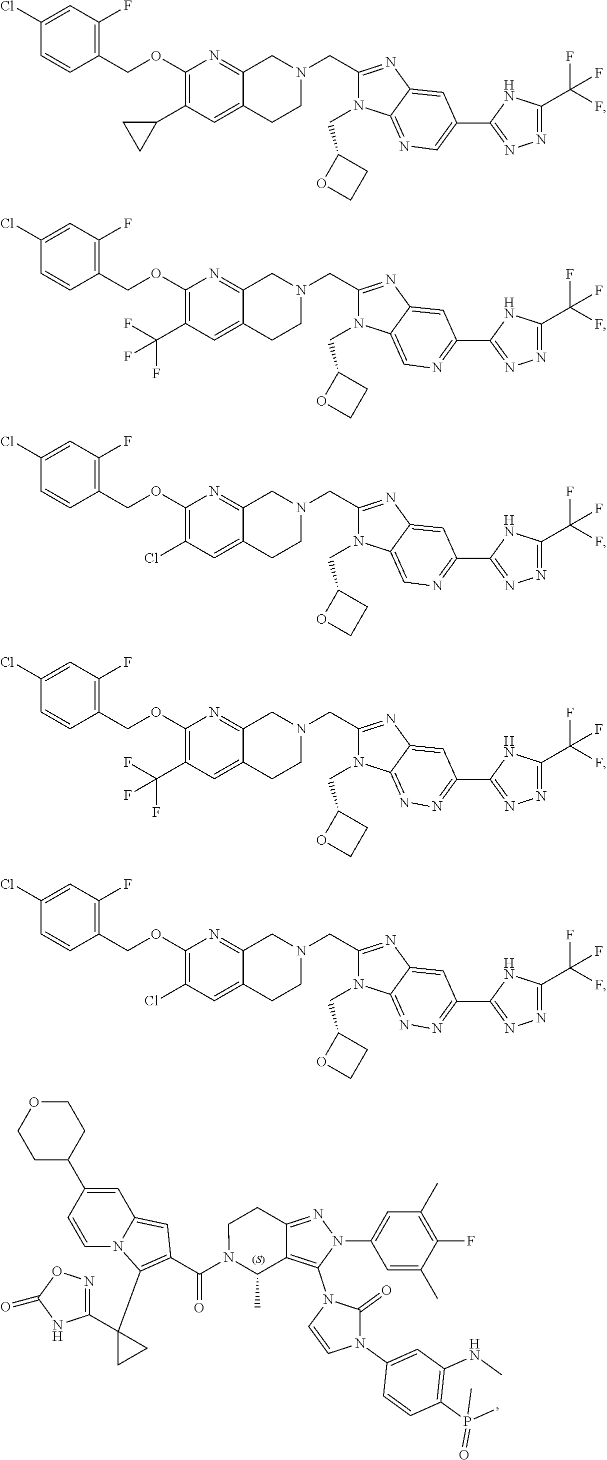

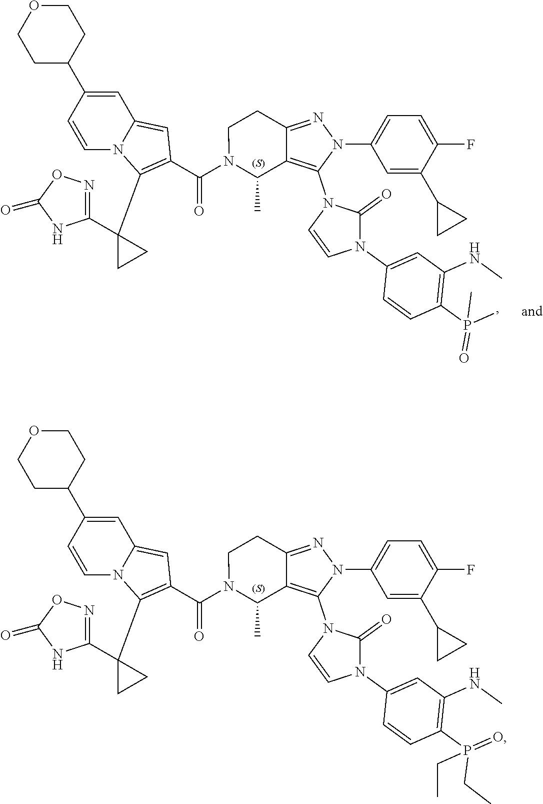

or a pharmaceutically acceptable salt thereof. In one embodiment, the GLP-1 modulating compound is selected from the group consisting of

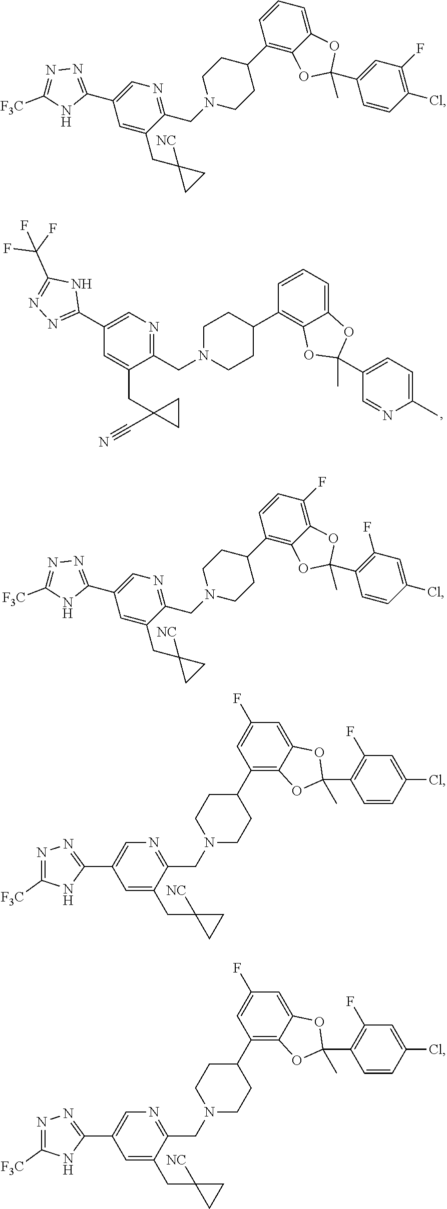 [SMILES: CC1(c2ccc(Cl)c(F)c2)Oc2cccc(C3CCN(Cc4ncc(-c5nnc(C(F)(F)F)[nH]5)cc4CC4(C#N)CC4)CC3)c2O1 ; Cc1ccc(C2(C)Oc3cccc(C4CCN(Cc5ncc(-c6nnc(C(F)(F)F)[nH]6)cc5CC5(C#N)CC5)CC4)c3O2)cn1 ; CC1(c2ccc(Cl)cc2F)Oc2c(F)ccc(C3CCN(Cc4ncc(-c5nnc(C(F)(F)F)[nH]5)cc4CC4(C#N)CC4)CC3)c2O1 ; CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1 ; CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1]

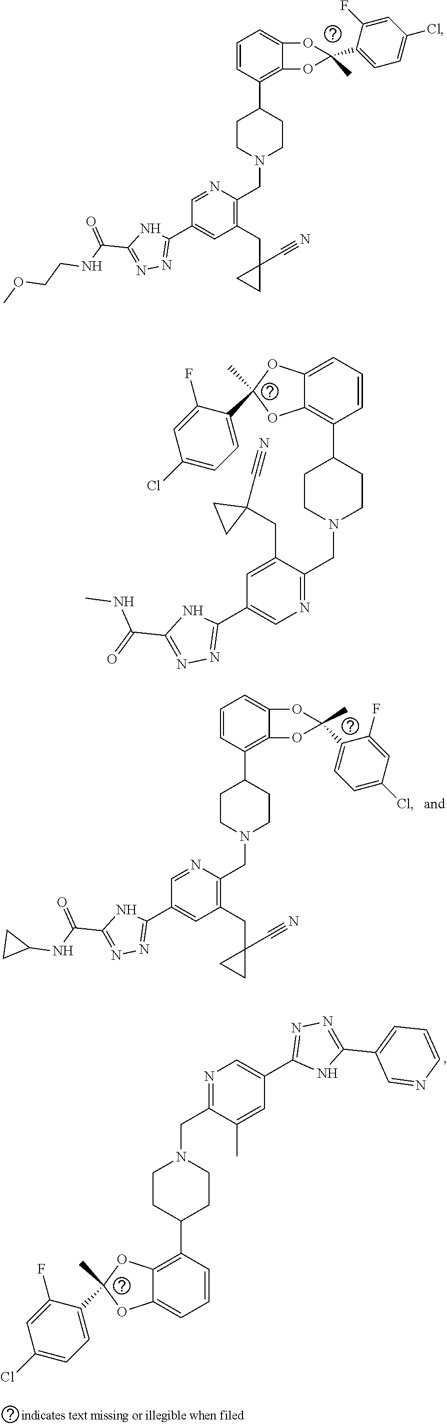 [SMILES: COCCNC(=O)c1nnc(-c2cnc(CN3CCC(c4cccc5c4O[C@@](C)(c4ccc(Cl)cc4F)O5)CC3)c(CC3(C#N)CC3)c2)[nH]1 ; CNC(=O)c1nnc(-c2cnc(CN3CCC(c4cccc5c4O[C@@](C)(c4ccc(Cl)cc4F)O5)CC3)c(CC3(C#N)CC3)c2)[nH]1 ; C[C@]1(c2ccc(Cl)cc2F)Oc2cccc(C3CCN(Cc4ncc(-c5nnc(C(=O)NC6CC6)[nH]5)cc4CC4(C#N)CC4)CC3)c2O1 ; "and" ; Cc1cc(-c2nnc(-c3cccnc3)[nH]2)cnc1CN1CCC(c2cccc3c2O[C@@](C)(c2ccc(Cl)cc2F)O3)CC1]

or a pharmaceutically acceptable salt thereof. In one embodiment, the GLP-1 modulating compound is selected from the group consisting of

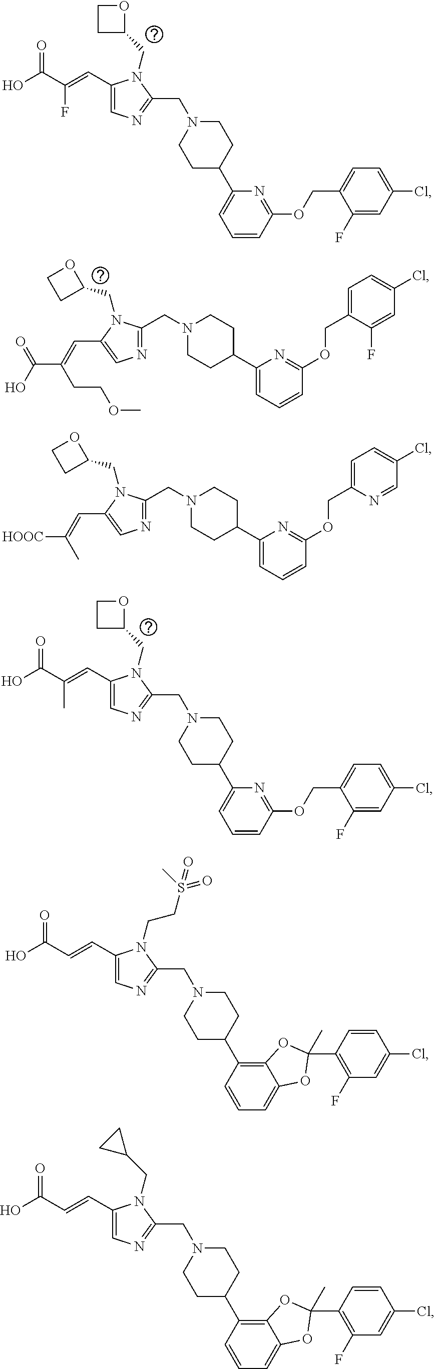 [SMILES: O=C(O)/C(F)=C/c1cnc(CN2CCC(c3cccc(OCc4ccc(Cl)cc4F)n3)CC2)n1C[C@@H]1CCO1 ; COCC/C(=C\c1cnc(CN2CCC(c3cccc(OCc4ccc(Cl)cc4F)n3)CC2)n1C[C@@H]1CCO1)C(=O)O ; C/C(=C\c1cnc(CN2CCC(c3cccc(OCc4ccc(Cl)cn4)n3)CC2)n1C[C@@H]1CCO1)C(=O)O ; C/C(=C\c1cnc(CN2CCC(c3cccc(OCc4ccc(Cl)cc4F)n3)CC2)n1C[C@@H]1CCO1)C(=O)O ; CC1(c2ccc(Cl)cc2F)Oc2cccc(C3CCN(Cc4ncc(/C=C/C(=O)O)n4CCS(C)(=O)=O)CC3)c2O1 ; CC1(c2ccc(Cl)cc2F)Oc2cccc(C3CCN(Cc4ncc(/C=C/C(=O)O)n4CC4CC4)CC3)c2O1]

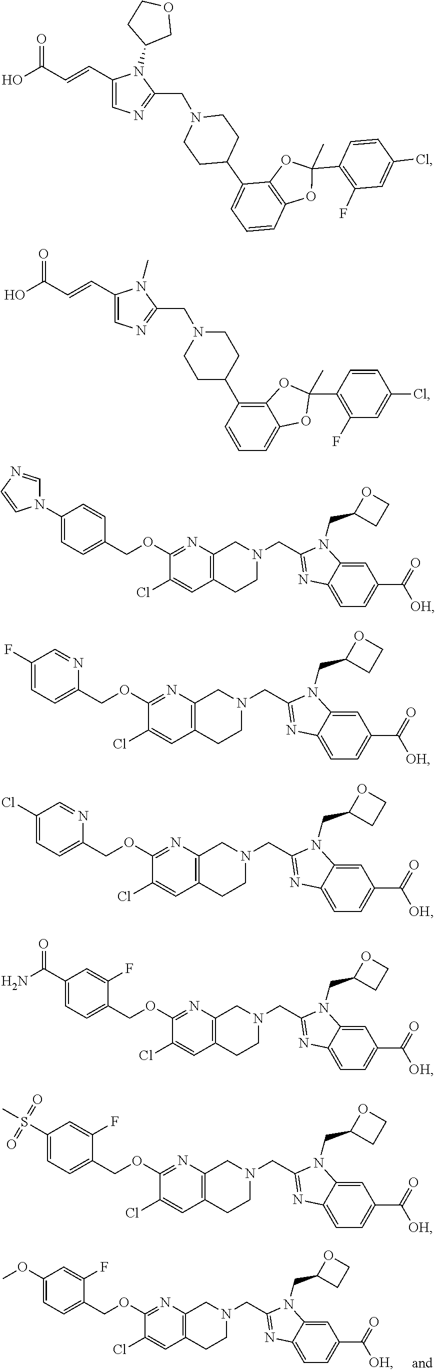

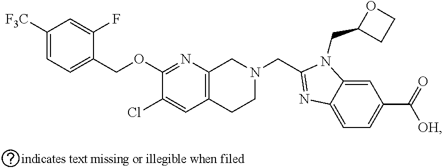 [SMILES: O=C(O)c1ccc2nc(CN3CCc4cc(Cl)c(OCc5ccc(C(F)(F)F)cc5F)nc4C3)n(C[C@@H]3CCO3)c2c1]

or a pharmaceutically acceptable salt thereof. In one embodiment, the GLP-1 modulating compound is selected from the group consisting of

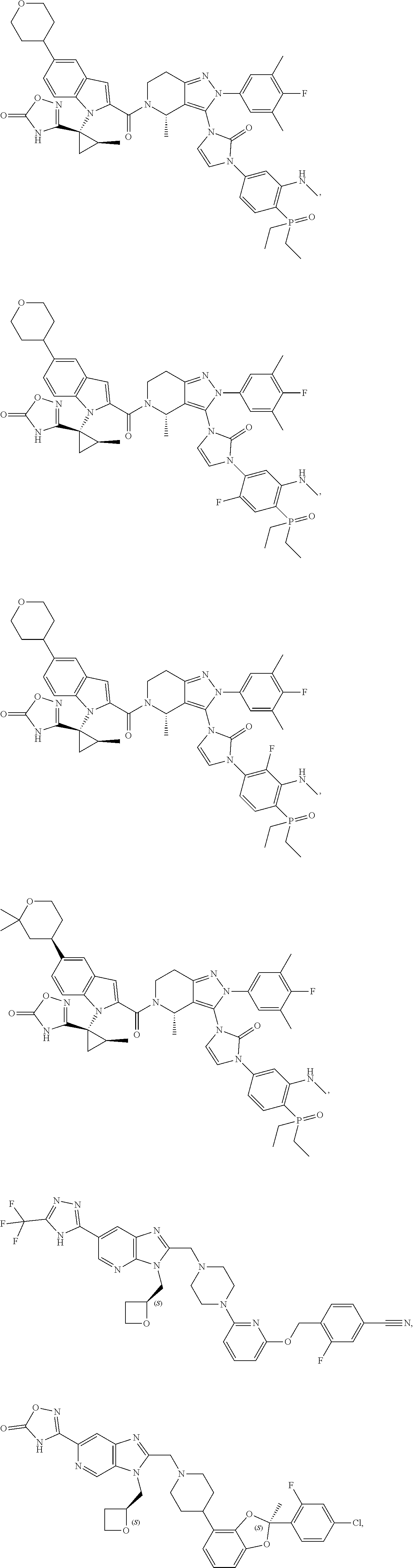

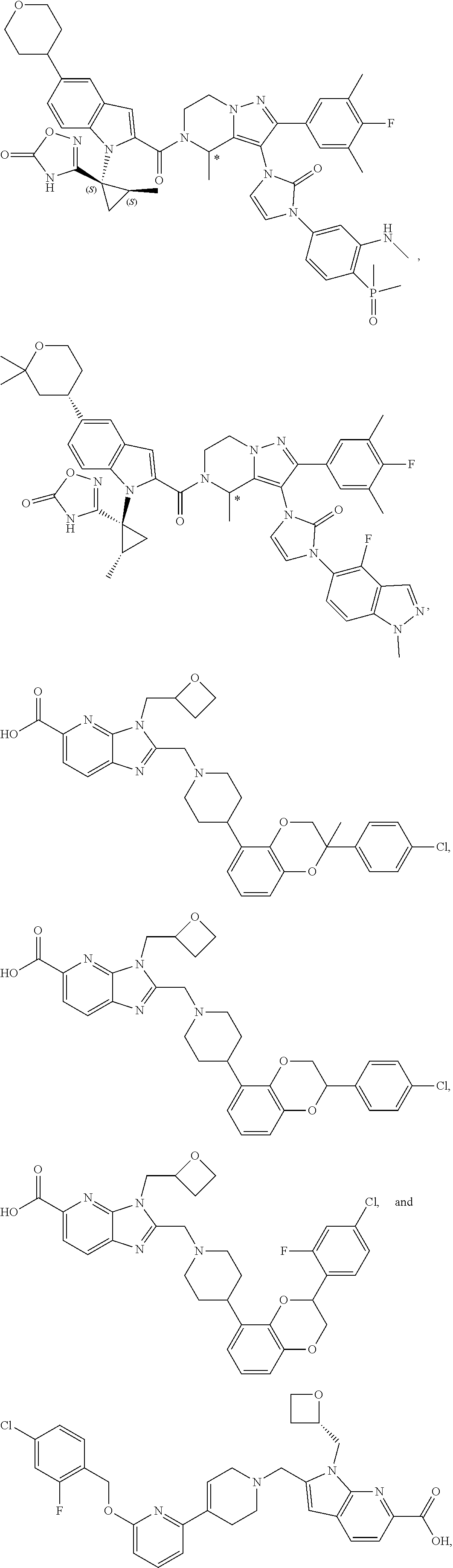

or a pharmaceutically acceptable salt thereof.

In one embodiment, the GLP-1 modulating compound is a compound disclosed in WO2021154796A1, WO2021081207A1, CN117279905A, U.S. Pat. No. 11,851,419B32, CN117295729A, or WO2022192430A1. In one embodiment, the GLP-1 modulating compound is selected from the group consisting of

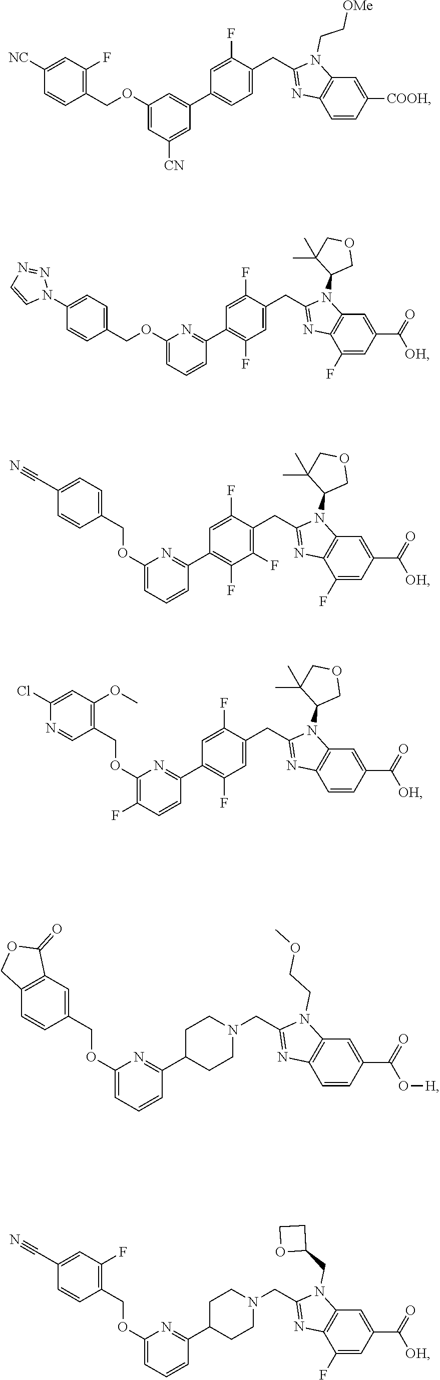

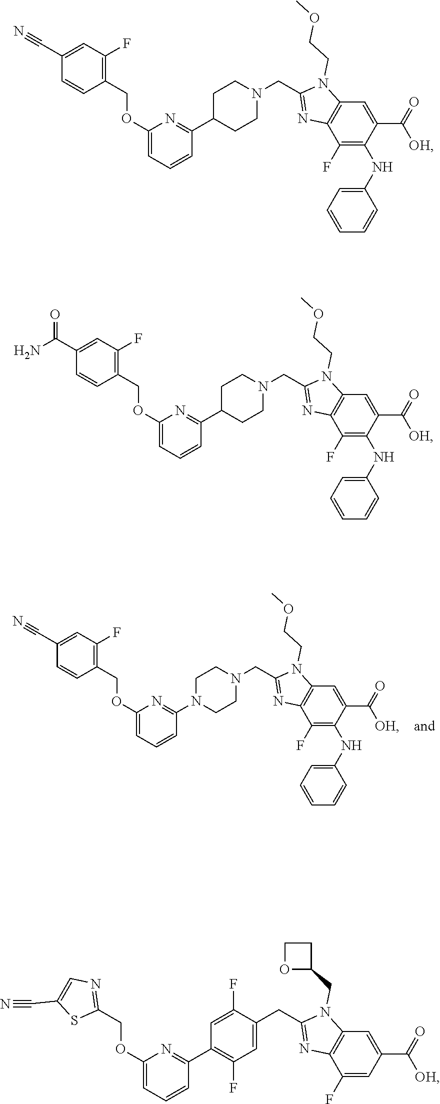 [SMILES: COCCn1c(CN2CCC(c3cccc(OCc4ccc(C#N)cc4F)n3)CC2)nc2c(F)c(Nc3ccccc3)c(C(=O)O)cc21 ; COCCn1c(CN2CCC(c3cccc(OCc4ccc(C(N)=O)cc4F)n3)CC2)nc2c(F)c(Nc3ccccc3)c(C(=O)O)cc21 ; COCCn1c(CN2CCN(c3cccc(OCc4ccc(C#N)cc4F)n3)CC2)nc2c(F)c(Nc3ccccc3)c(C(=O)O)cc21 ; "and" ; N#Cc1cnc(COc2cccc(-c3cc(F)c(Cc4nc5c(F)cc(C(=O)O)cc5n4C[C@@H]4CCO4)cc3F)n2)s1]

or a pharmaceutically acceptable salt thereof.

In one embodiment, the GLP-1 modulating compound is a compound disclosed in CN113831337B. In one embodiment, the GLP-1 modulating compound is selected from the group consisting of

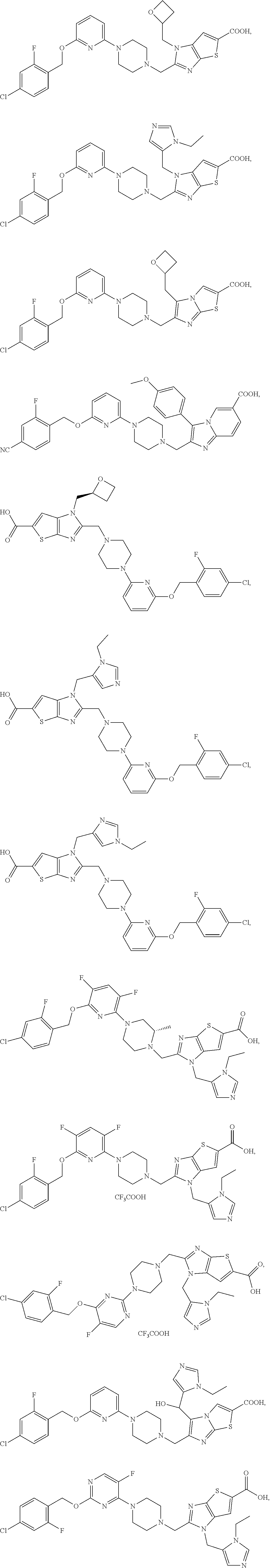

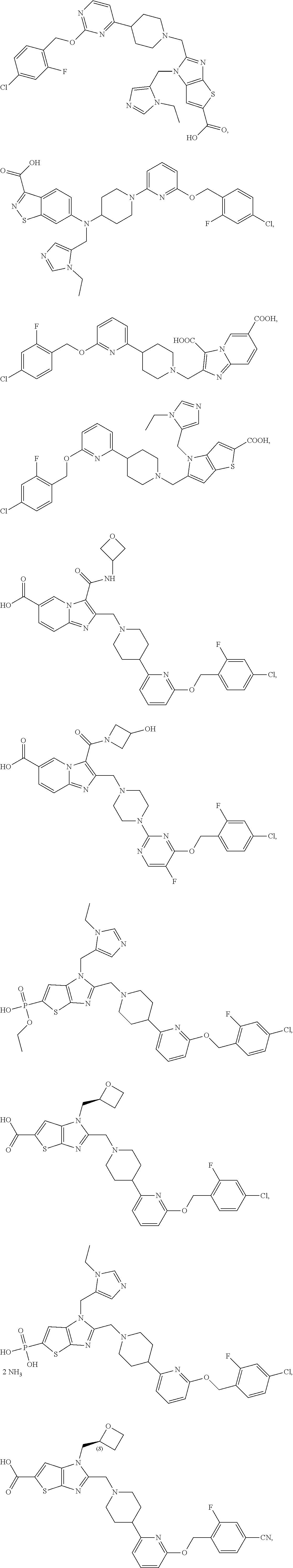

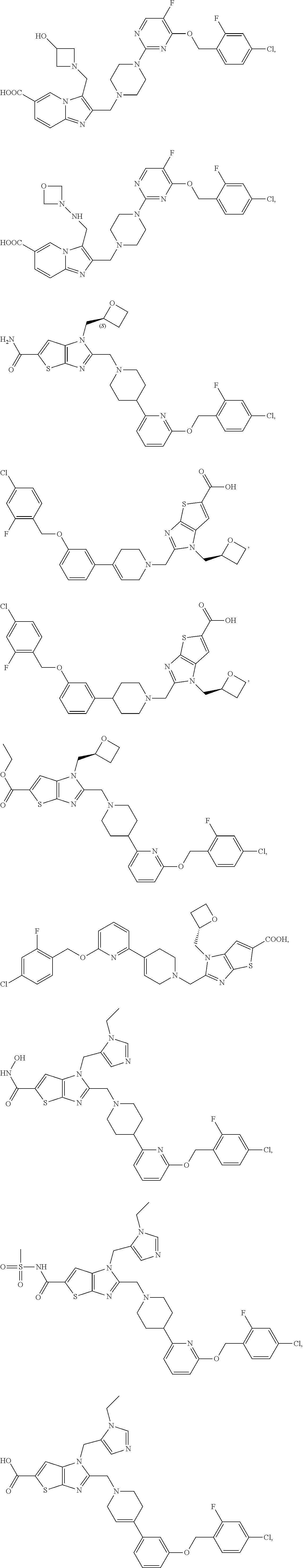

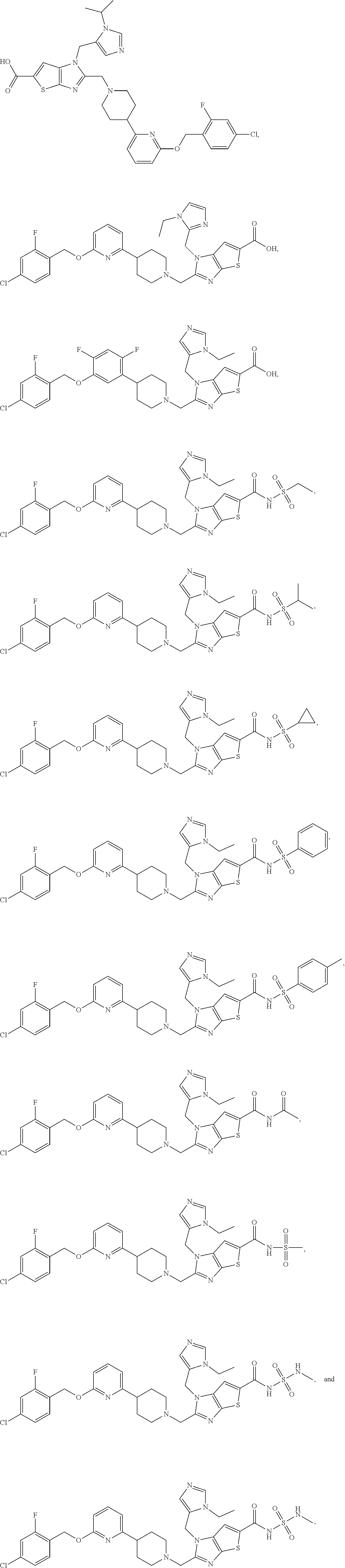

or a pharmaceutically acceptable salt thereof.

In one embodiment, the GLP-1 modulating compound is a compound disclosed in WO2023051490A1, WO2022268152A1, CN117098758A, WO2022199458A1, CN114907351A, CN114478497A, CN116940561A, CN114478497B, or EP4227299A1. In one embodiment, the GLP-1 modulating compound is selected from the group consisting of

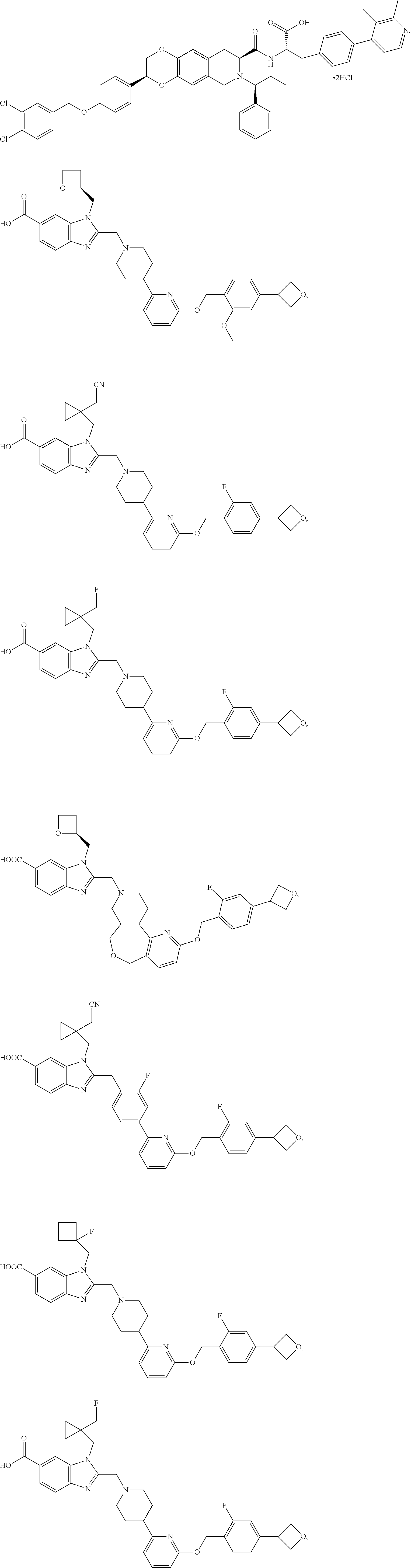

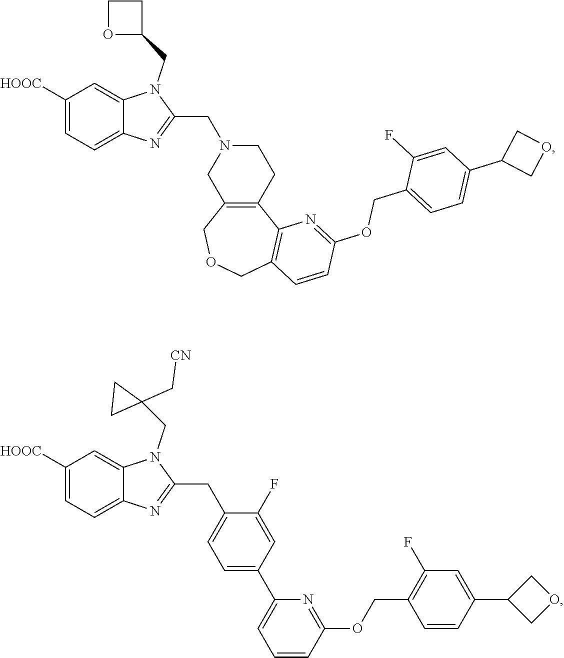 [SMILES: O=C(O)c1ccc2nc(CN3CCC4=C(COCc5ccc(OCc6ccc(C7COC7)cc6F)nc54)C3)n(C[C@@H]3CCO3)c2c1 ; N#CCC1(Cn2c(Cc3ccc(-c4cccc(OCc5ccc(C6COC6)cc5F)n4)cc3F)nc3ccc(C(=O)O)cc32)CC1]

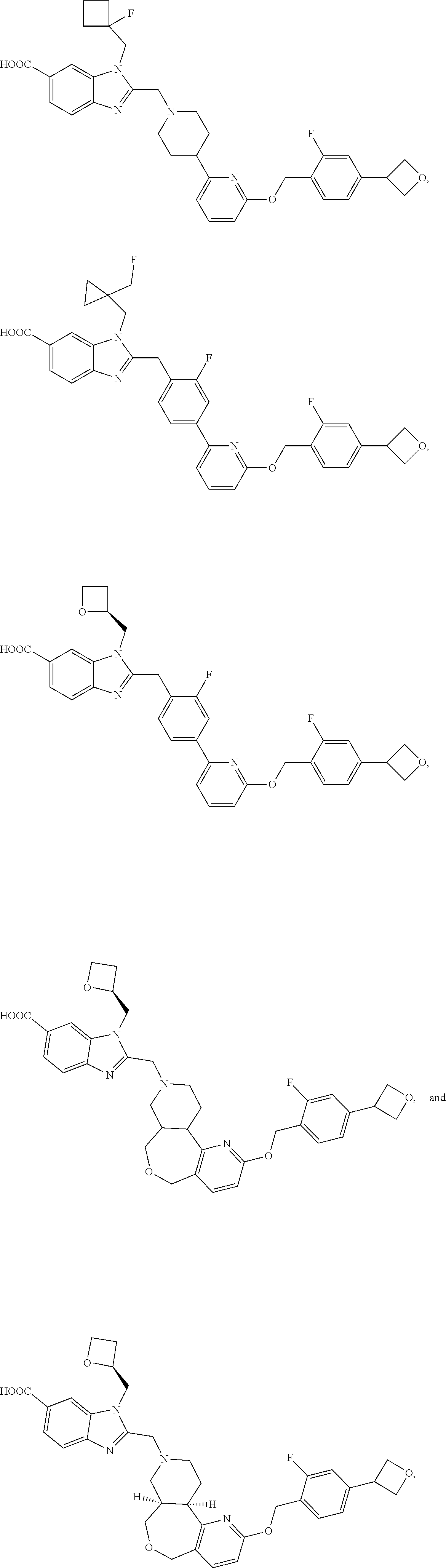 [SMILES: O=C(O)c1ccc2nc(CN3CCC(c4cccc(OCc5ccc(C6COC6)cc5F)n4)CC3)n(CC3(F)CCC3)c2c1 ; O=C(O)c1ccc2nc(Cc3ccc(-c4cccc(OCc5ccc(C6COC6)cc5F)n4)cc3F)n(CC3(CF)CC3)c2c1 ; O=C(O)c1ccc2nc(Cc3ccc(-c4cccc(OCc5ccc(C6COC6)cc5F)n4)cc3F)n(C[C@@H]3CCO3)c2c1 ; O=C(O)c1ccc2nc(CN3CCC4c5nc(OCc6ccc(C7COC7)cc6F)ccc5COCC4C3)n(C[C@@H]3CCO3)c2c1 ; "and" ; O=C(O)c1ccc2nc(CN3CC[C@H]4c5nc(OCc6ccc(C7COC7)cc6F)ccc5COC[C@H]4C3)n(C[C@@H]3CCO3)c2c1]

or a pharmaceutically acceptable salt thereof. In one embodiment, the GLP-1 modulating compound is selected from the group consisting of

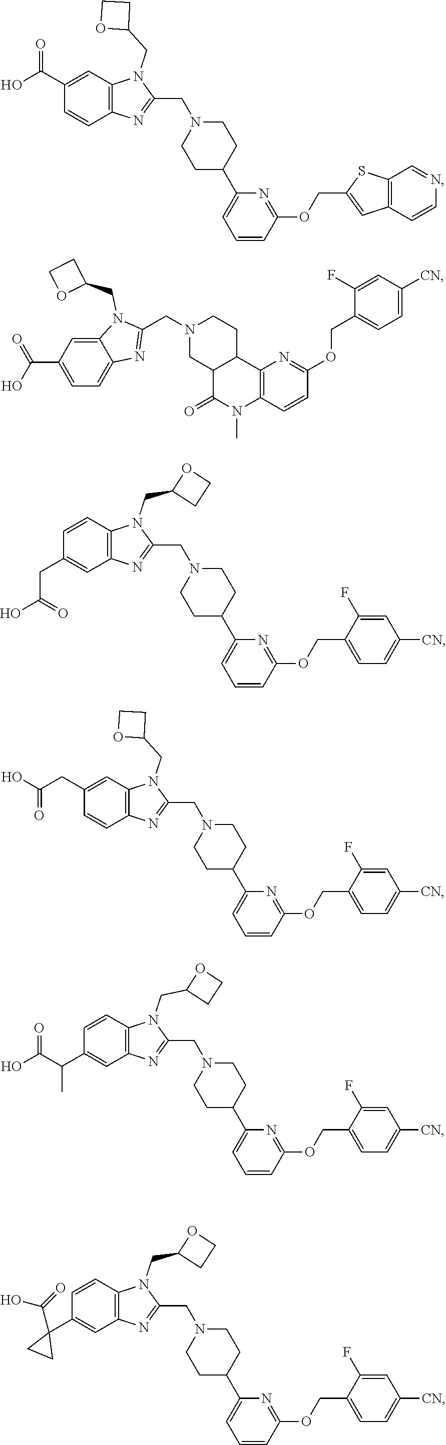 [SMILES: O=C(O)c1ccc2nc(CN3CCC(c4cccc(OCc5cc6ccncc6s5)n4)CC3)n(CC3CCO3)c2c1 ; CN1C(=O)C2CN(Cc3nc4ccc(C(=O)O)cc4n3C[C@@H]3CCO3)CCC2c2nc(OCc3ccc(C#N)cc3F)ccc21 ; N#Cc1ccc(COc2cccc(C3CCN(Cc4nc5cc(CC(=O)O)ccc5n4C[C@@H]4CCO4)CC3)n2)c(F)c1 ; N#Cc1ccc(COc2cccc(C3CCN(Cc4nc5ccc(CC(=O)O)cc5n4CC4CCO4)CC3)n2)c(F)c1 ; CC(C(=O)O)c1ccc2c(c1)nc(CN1CCC(c3cccc(OCc4ccc(C#N)cc4F)n3)CC1)n2CC1CCO1 ; N#Cc1ccc(COc2cccc(C3CCN(Cc4nc5cc(C6(C(=O)O)CC6)ccc5n4C[C@@H]4CCO4)CC3)n2)c(F)c1]

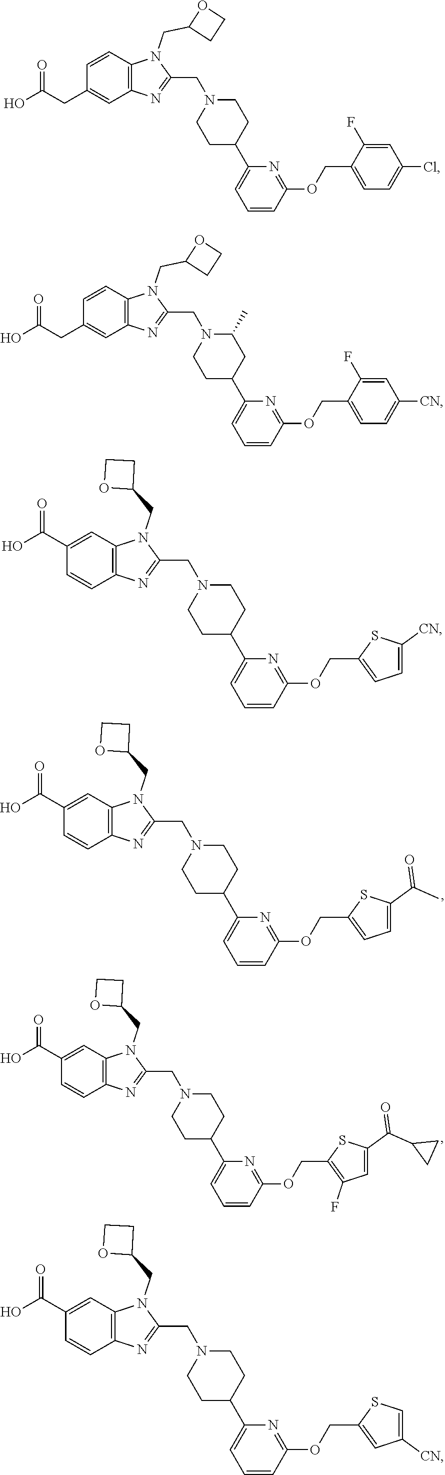 [SMILES: O=C(O)Cc1ccc2c(c1)nc(CN1CCC(c3cccc(OCc4ccc(Cl)cc4F)n3)CC1)n2CC1CCO1 ; C[C@@H]1CC(c2cccc(OCc3ccc(C#N)cc3F)n2)CCN1Cc1nc2cc(CC(=O)O)ccc2n1CC1CCO1 ; N#Cc1ccc(COc2cccc(C3CCN(Cc4nc5ccc(C(=O)O)cc5n4C[C@@H]4CCO4)CC3)n2)s1 ; CC(=O)c1ccc(COc2cccc(C3CCN(Cc4nc5ccc(C(=O)O)cc5n4C[C@@H]4CCO4)CC3)n2)s1 ; O=C(O)c1ccc2nc(CN3CCC(c4cccc(OCc5sc(C(=O)C6CC6)cc5F)n4)CC3)n(C[C@@H]3CCO3)c2c1 ; N#Cc1csc(COc2cccc(C3CCN(Cc4nc5ccc(C(=O)O)cc5n4C[C@@H]4CCO4)CC3)n2)c1]

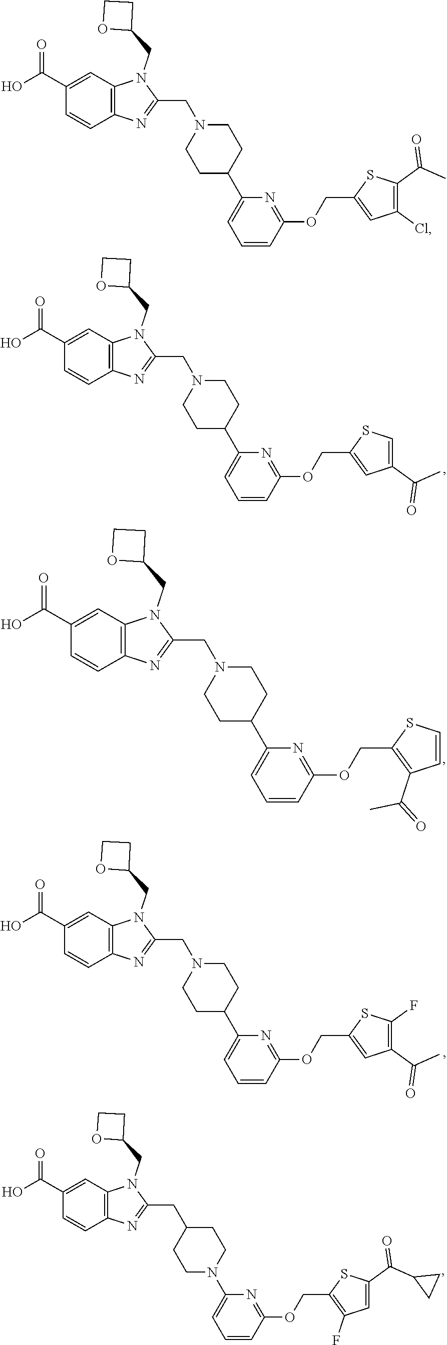 [SMILES: CC(=O)c1sc(COc2cccc(C3CCN(Cc4nc5ccc(C(=O)O)cc5n4C[C@@H]4CCO4)CC3)n2)cc1Cl ; CC(=O)c1csc(COc2cccc(C3CCN(Cc4nc5ccc(C(=O)O)cc5n4C[C@@H]4CCO4)CC3)n2)c1 ; CC(=O)c1ccsc1COc1cccc(C2CCN(Cc3nc4ccc(C(=O)O)cc4n3C[C@@H]3CCO3)CC2)n1 ; CC(=O)c1cc(COc2cccc(C3CCN(Cc4nc5ccc(C(=O)O)cc5n4C[C@@H]4CCO4)CC3)n2)sc1F ; O=C(O)c1ccc2nc(CC3CCN(c4cccc(OCc5sc(C(=O)C6CC6)cc5F)n4)CC3)n(C[C@@H]3CCO3)c2c1]

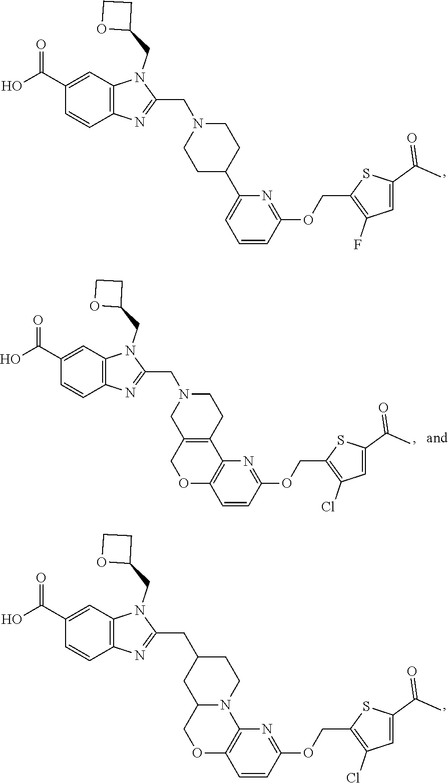 [SMILES: CC(=O)c1cc(F)c(COc2cccc(C3CCN(Cc4nc5ccc(C(=O)O)cc5n4C[C@@H]4CCO4)CC3)n2)s1 ; CC(=O)c1cc(Cl)c(COc2ccc3c(n2)C2=C(CO3)CN(Cc3nc4ccc(C(=O)O)cc4n3C[C@@H]3CCO3)CC2)s1 ; CC(=O)c1cc(Cl)c(COc2ccc3c(n2)N2CCC(Cc4nc5ccc(C(=O)O)cc5n4C[C@@H]4CCO4)CC2CO3)s1]

or a pharmaceutically acceptable salt thereof. In one embodiment, the GLP-1 modulating compound is selected from the group consisting of:

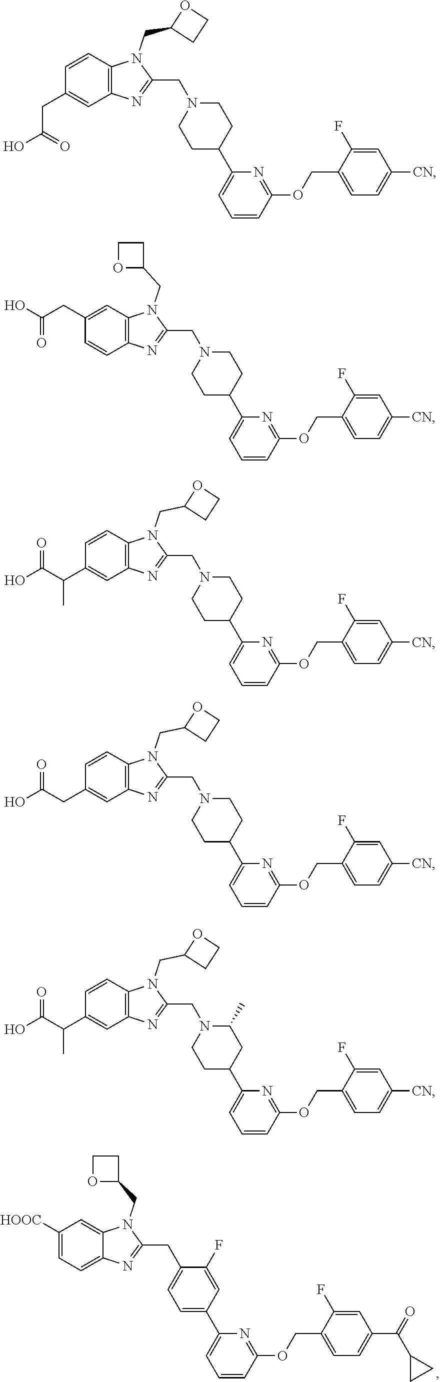 [SMILES: N#Cc1ccc(COc2cccc(C3CCN(Cc4nc5cc(CC(=O)O)ccc5n4C[C@@H]4CCO4)CC3)n2)c(F)c1 ; N#Cc1ccc(COc2cccc(C3CCN(Cc4nc5ccc(CC(=O)O)cc5n4CC4CCO4)CC3)n2)c(F)c1 ; CC(C(=O)O)c1ccc2c(c1)nc(CN1CCC(c3cccc(OCc4ccc(C#N)cc4F)n3)CC1)n2CC1CCO1 ; N#Cc1ccc(COc2cccc(C3CCN(Cc4nc5cc(CC(=O)O)ccc5n4CC4CCO4)CC3)n2)c(F)c1 ; CC(C(=O)O)c1ccc2c(c1)nc(CN1CCC(c3cccc(OCc4ccc(C#N)cc4F)n3)C[C@H]1C)n2CC1CCO1 ; O=C(O)c1ccc2nc(Cc3ccc(-c4cccc(OCc5ccc(C(=O)C6CC6)cc5F)n4)cc3F)n(C[C@@H]3CCO3)c2c1]

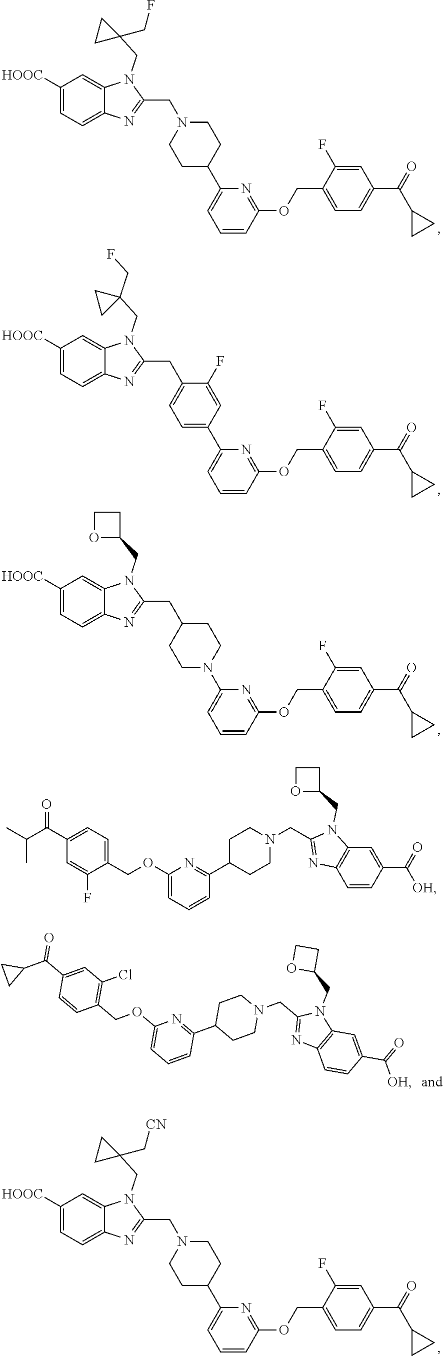 [SMILES: O=C(O)c1ccc2nc(CN3CCC(c4cccc(OCc5ccc(C(=O)C6CC6)cc5F)n4)CC3)n(CC3(CF)CC3)c2c1 ; O=C(O)c1ccc2nc(Cc3ccc(-c4cccc(OCc5ccc(C(=O)C6CC6)cc5F)n4)cc3F)n(CC3(CF)CC3)c2c1 ; O=C(O)c1ccc2nc(CC3CCN(c4cccc(OCc5ccc(C(=O)C6CC6)cc5F)n4)CC3)n(C[C@@H]3CCO3)c2c1 ; CC(C)C(=O)c1ccc(COc2cccc(C3CCN(Cc4nc5ccc(C(=O)O)cc5n4C[C@@H]4CCO4)CC3)n2)c(F)c1 ; O=C(O)c1ccc2nc(CN3CCC(c4cccc(OCc5ccc(C(=O)C6CC6)cc5Cl)n4)CC3)n(C[C@@H]3CCO3)c2c1 ; N#CCC1(Cn2c(CN3CCC(c4cccc(OCc5ccc(C(=O)C6CC6)cc5F)n4)CC3)nc3ccc(C(=O)O)cc32)CC1]

or a pharmaceutically acceptable salt thereof.

In one embodiment, the GLP-1 modulating compound is a compound disclosed in WO2021112538A1. In one embodiment, the GLP-1 modulating compound is

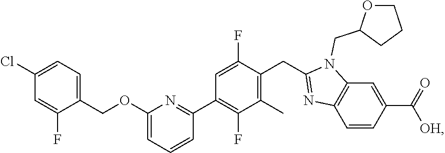 [SMILES: Cc1c(F)c(-c2cccc(OCc3ccc(Cl)cc3F)n2)cc(F)c1Cc1nc2ccc(C(=O)O)cc2n1CC1CCCO1]

or a pharmaceutically acceptable salt thereof.

In one embodiment, the GLP-1 modulating compound is a compound disclosed in WO2021096304A1, WO2023182869A1, US20230212140A1, or TW1817870B, WO2021096284A1.

In one embodiment, the GLP-1 modulating compound is selected from the group consisting of

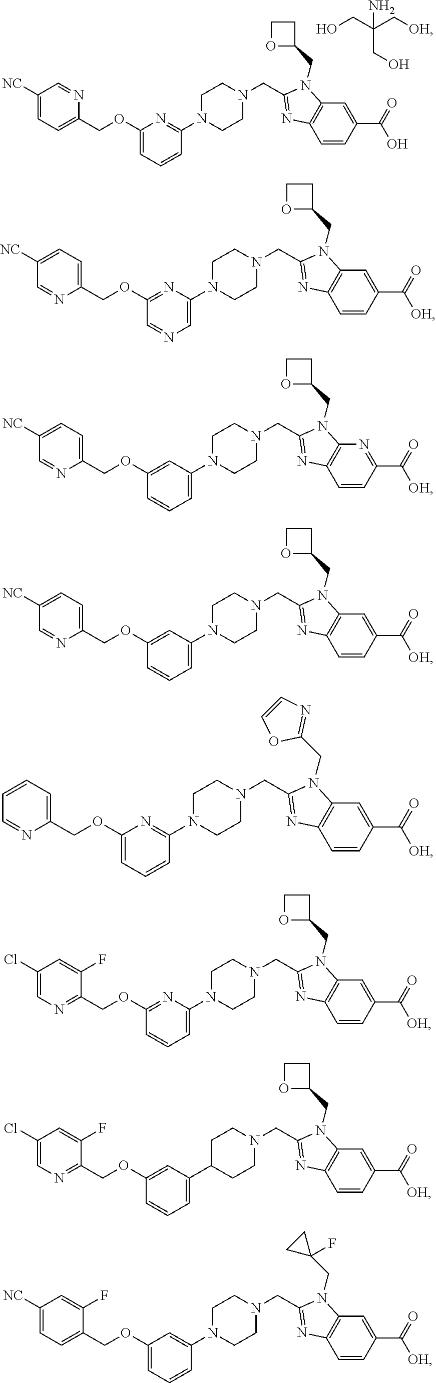

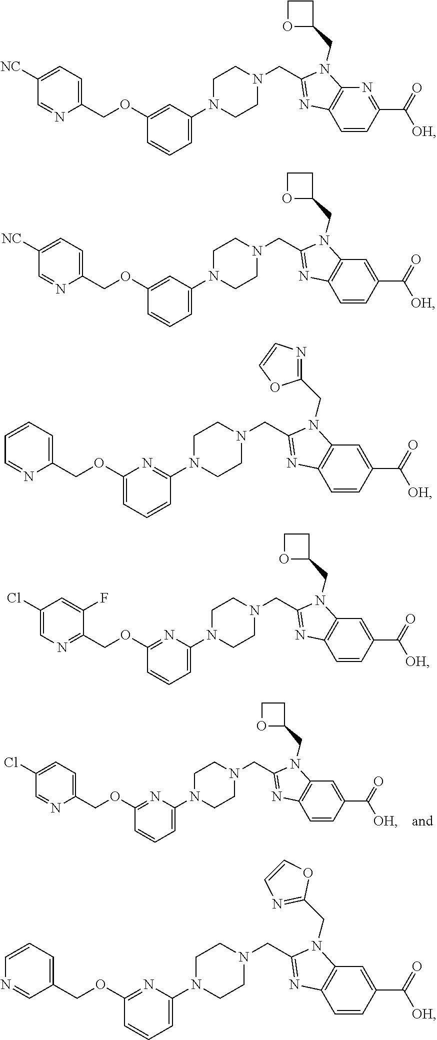

or a pharmaceutically acceptable salt thereof.

In one embodiment, the GLP-1 modulating compound is a compound disclosed in CN116102543A. In one embodiment, the GLP-1 modulating compound is selected from the group consisting of

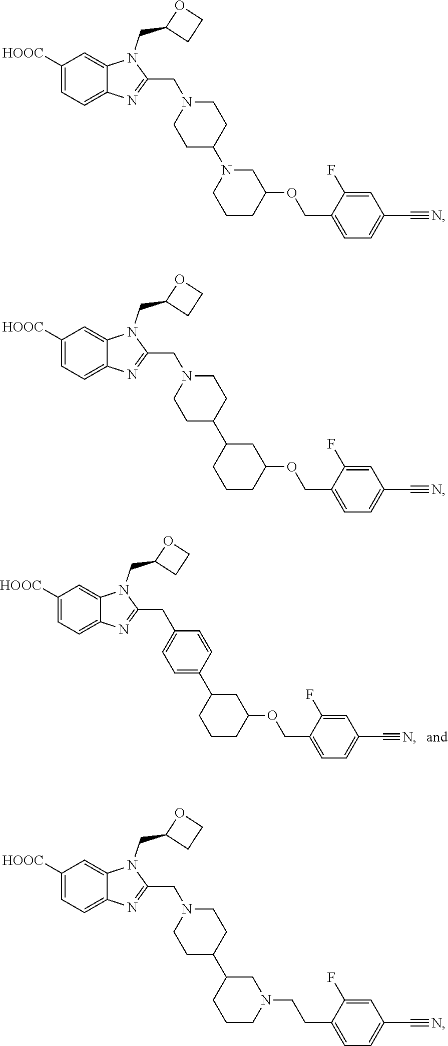 [SMILES: N#Cc1ccc(COC2CCCN(C3CCN(Cc4nc5ccc(C(=O)O)cc5n4C[C@@H]4CCO4)CC3)C2)c(F)c1 ; N#Cc1ccc(COC2CCCC(C3CCN(Cc4nc5ccc(C(=O)O)cc5n4C[C@@H]4CCO4)CC3)C2)c(F)c1 ; N#Cc1ccc(COC2CCCC(c3ccc(Cc4nc5ccc(C(=O)O)cc5n4C[C@@H]4CCO4)cc3)C2)c(F)c1 ; N#Cc1ccc(CCN2CCCC(C3CCN(Cc4nc5ccc(C(=O)O)cc5n4C[C@@H]4CCO4)CC3)C2)c(F)c1]

or a pharmaceutically acceptable salt thereof.

In one embodiment, the GLP-1 modulating compound is a compound disclosed in CN113773310A. In one embodiment, the GLP-1 modulating compound is selected from the group consisting of

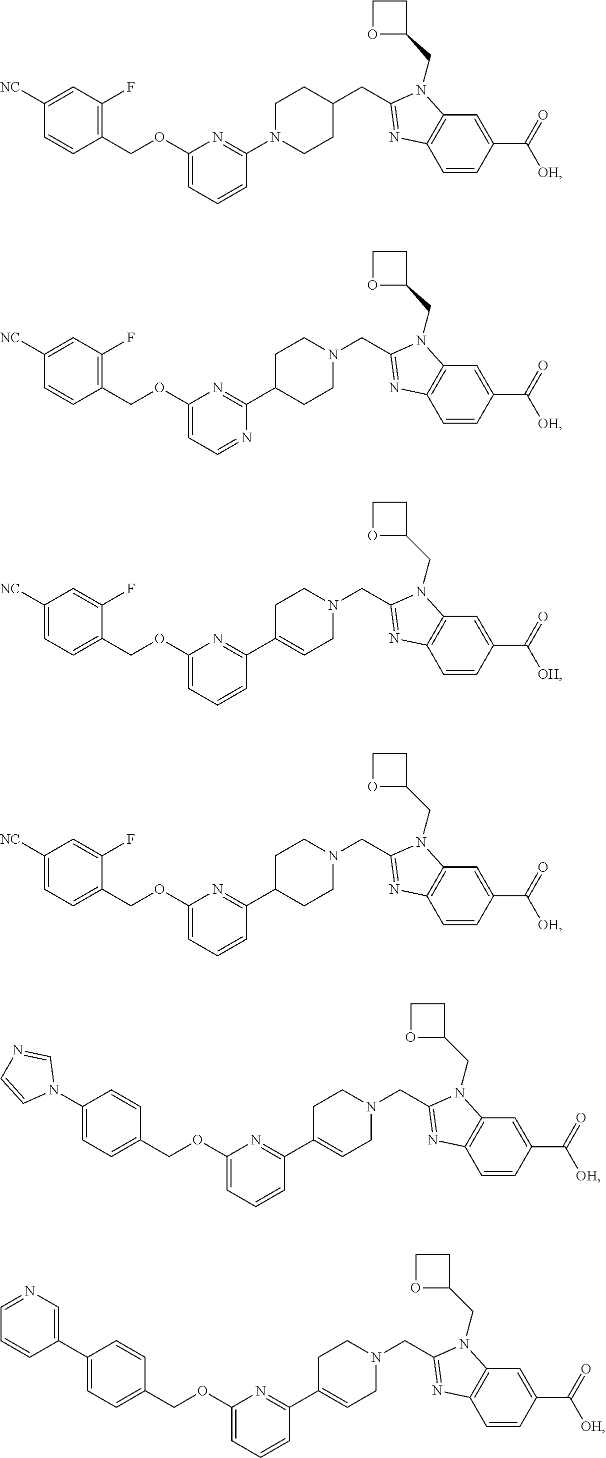 [SMILES: N#Cc1ccc(COc2cccc(N3CCC(Cc4nc5ccc(C(=O)O)cc5n4C[C@@H]4CCO4)CC3)n2)c(F)c1 ; N#Cc1ccc(COc2ccnc(C3CCN(Cc4nc5ccc(C(=O)O)cc5n4C[C@@H]4CCO4)CC3)n2)c(F)c1 ; N#Cc1ccc(COc2cccc(C3=CCN(Cc4nc5ccc(C(=O)O)cc5n4CC4CCO4)CC3)n2)c(F)c1 ; N#Cc1ccc(COc2cccc(C3CCN(Cc4nc5ccc(C(=O)O)cc5n4CC4CCO4)CC3)n2)c(F)c1 ; O=C(O)c1ccc2nc(CN3CC=C(c4cccc(OCc5ccc(-n6ccnc6)cc5)n4)CC3)n(CC3CCO3)c2c1 ; O=C(O)c1ccc2nc(CN3CC=C(c4cccc(OCc5ccc(-c6cccnc6)cc5)n4)CC3)n(CC3CCO3)c2c1]

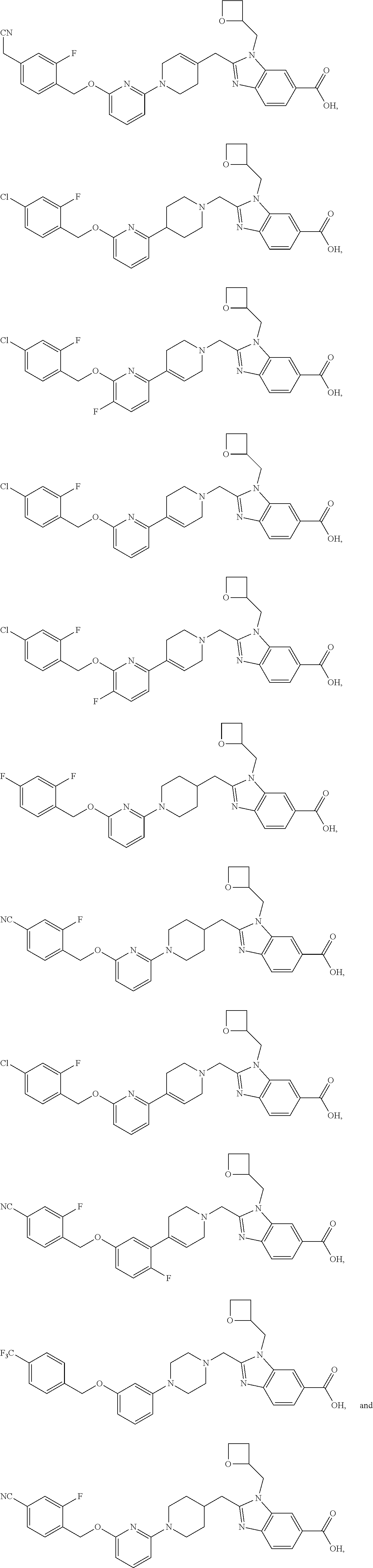

or a pharmaceutically acceptable salt thereof.

In one embodiment, the GLP-1 modulating compound is a compound disclosed in WO2022184849A1. In one embodiment, the GLP-1 modulating compound is

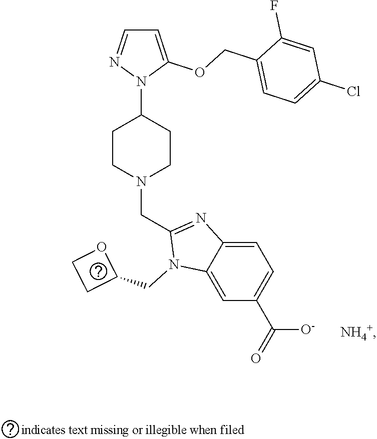 [SMILES: O=C([O-])c1ccc2nc(CN3CCC(n4nccc4OCc4ccc(Cl)cc4F)CC3)n(C[C@@H]3CCO3)c2c1]

or a pharmaceutically acceptable salt thereof.

In one embodiment, the GLP-1 modulating compound is a compound disclosed in JP2023520181A, CN117279904A, US20230203021A1, or EP4303215A1. In one embodiment, the GLP-1 modulating compound is selected from the group consisting of

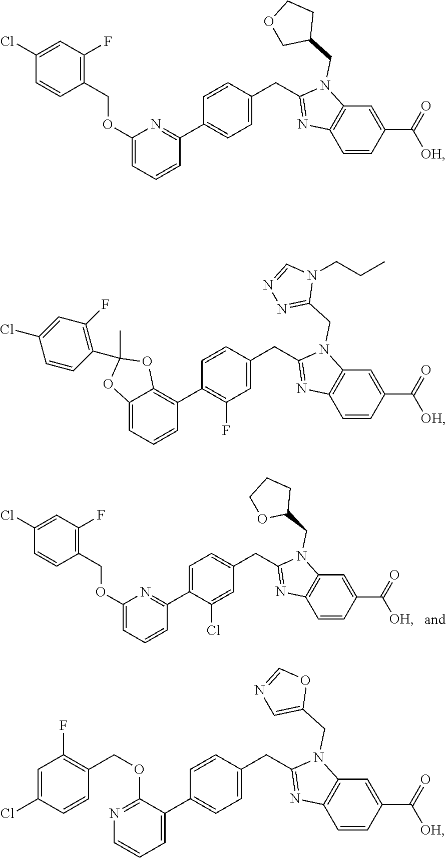

or a pharmaceutically acceptable salt thereof.

In one embodiment, the GLP-1 modulating compound is a compound disclosed in EP4166142A1 or WO2023029380A1. In one embodiment, the GLP-1 modulating compound is selected from the group consisting of

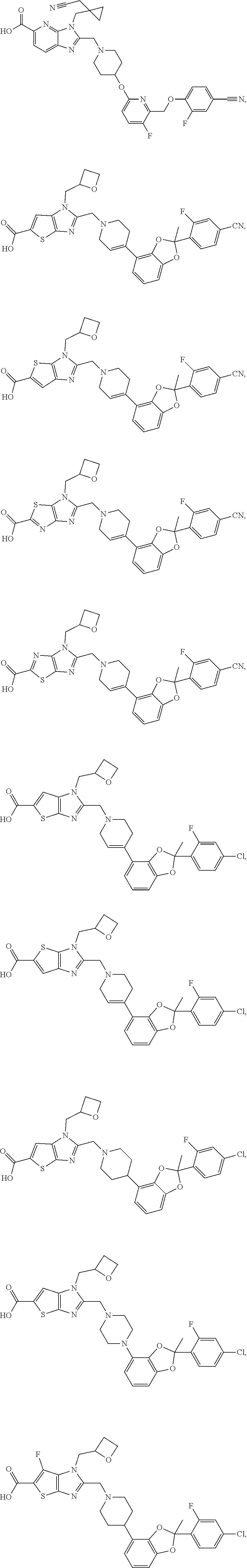

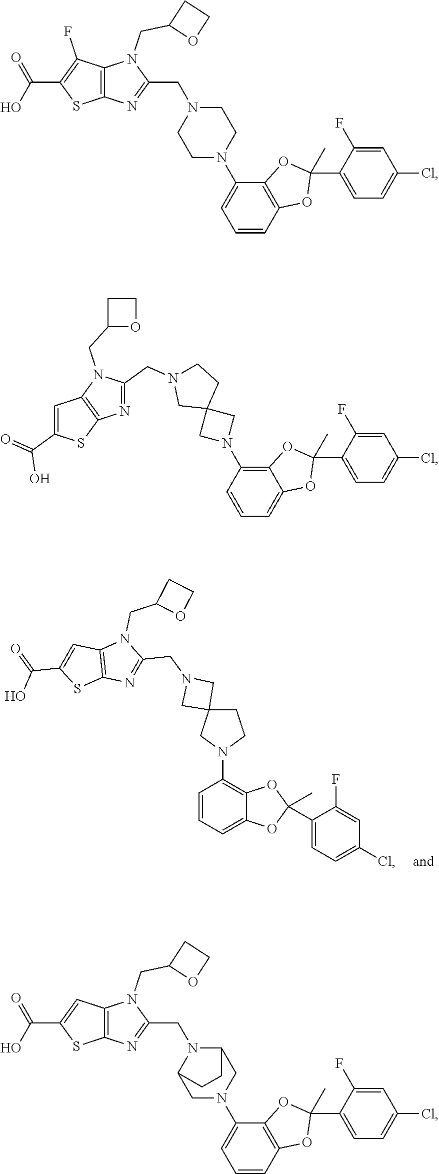 [SMILES: CC1(c2ccc(Cl)cc2F)Oc2cccc(N3CCN(Cc4nc5sc(C(=O)O)c(F)c5n4CC4CCO4)CC3)c2O1 ; CC1(c2ccc(Cl)cc2F)Oc2cccc(N3CC4(CCN(Cc5nc6sc(C(=O)O)cc6n5CC5CCO5)C4)C3)c2O1 ; CC1(c2ccc(Cl)cc2F)Oc2cccc(N3CCC4(CN(Cc5nc6sc(C(=O)O)cc6n5CC5CCO5)C4)C3)c2O1 ; "and" ; CC1(c2ccc(Cl)cc2F)Oc2cccc(N3CC4CCC(C3)N4Cc3nc4sc(C(=O)O)cc4n3CC3CCO3)c2O1]

or a pharmaceutically acceptable salt thereof.

In one embodiment, the GLP-1 modulating compound is a compound disclosed in WO2022219495A1, WO2023152698A1, or CN117222644A. In one embodiment, the GLP-1 modulating compound is selected from the group consisting of

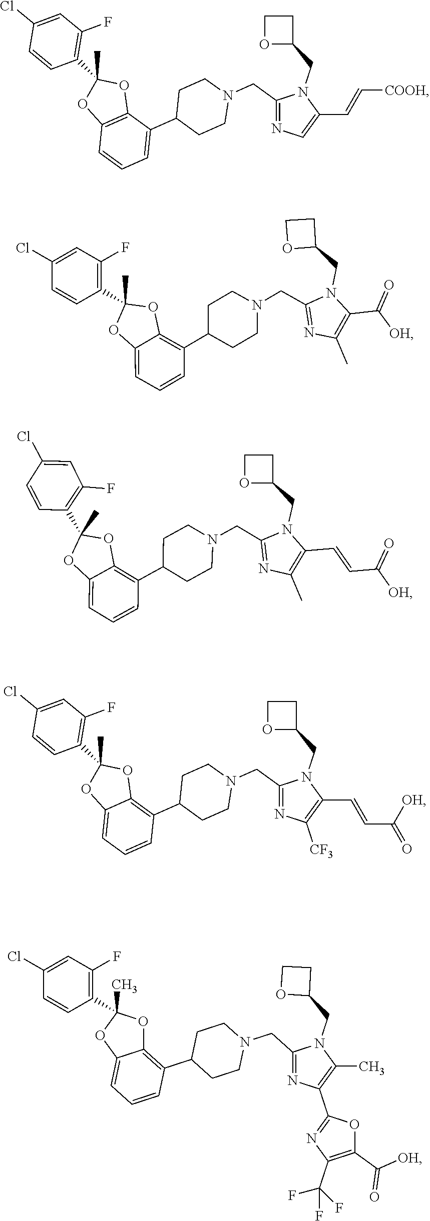 [SMILES: C[C@]1(c2ccc(Cl)cc2F)Oc2cccc(C3CCN(Cc4ncc(/C=C/C(=O)O)n4C[C@@H]4CCO4)CC3)c2O1 ; Cc1nc(CN2CCC(c3cccc4c3O[C@@](C)(c3ccc(Cl)cc3F)O4)CC2)n(C[C@@H]2CCO2)c1C(=O)O ; Cc1nc(CN2CCC(c3cccc4c3O[C@@](C)(c3ccc(Cl)cc3F)O4)CC2)n(C[C@@H]2CCO2)c1/C=C/C(=O)O ; C[C@]1(c2ccc(Cl)cc2F)Oc2cccc(C3CCN(Cc4nc(C(F)(F)F)c(/C=C/C(=O)O)n4C[C@@H]4CCO4)CC3)c2O1 ; Cc1c(-c2nc(C(F)(F)F)c(C(=O)O)o2)nc(CN2CCC(c3cccc4c3O[C@@](C)(c3ccc(Cl)cc3F)O4)CC2)n1C[C@@H]1CCO1]

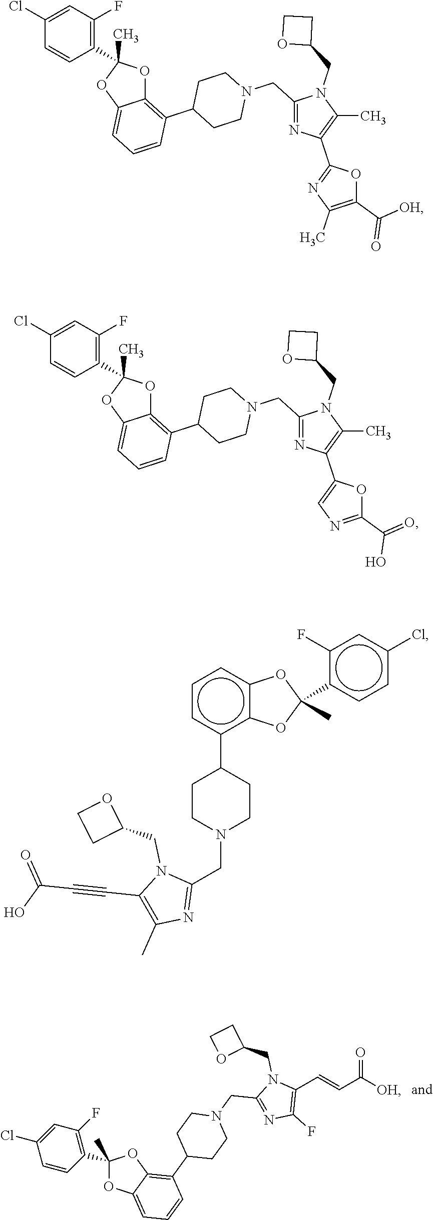

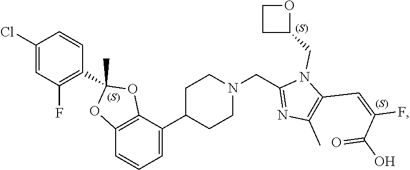

or a pharmaceutically acceptable salt thereof.

In one embodiment, the GLP-1 modulating compound is a compound disclosed in WO2022031994A1, WO2020207474A1, WO2021242817A1, CN113227068B3, WO2020103815A1, US2020028342A1, or US20230278991A1. In one embodiment, the GLP-1 modulating compound is selected from the group consisting of:

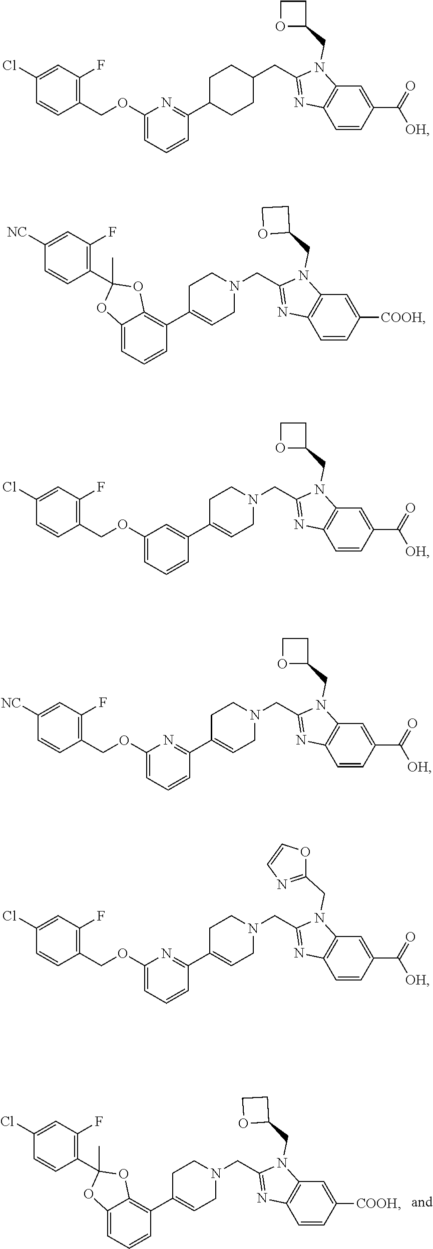 [SMILES: O=C(O)c1ccc2nc(CC3CCC(c4cccc(OCc5ccc(Cl)cc5F)n4)CC3)n(C[C@@H]3CCO3)c2c1 ; CC1(c2ccc(C#N)cc2F)Oc2cccc(C3=CCN(Cc4nc5ccc(C(=O)O)cc5n4C[C@@H]4CCO4)CC3)c2O1 ; O=C(O)c1ccc2nc(CN3CC=C(c4cccc(OCc5ccc(Cl)cc5F)c4)CC3)n(C[C@@H]3CCO3)c2c1 ; N#Cc1ccc(COc2cccc(C3=CCN(Cc4nc5ccc(C(=O)O)cc5n4C[C@@H]4CCO4)CC3)n2)c(F)c1 ; O=C(O)c1ccc2nc(CN3CC=C(c4cccc(OCc5ccc(Cl)cc5F)n4)CC3)n(Cc3ncco3)c2c1 ; CC1(c2ccc(Cl)cc2F)Oc2cccc(C3=CCN(Cc4nc5ccc(C(=O)O)cc5n4C[C@@H]4CCO4)CC3)c2O1]

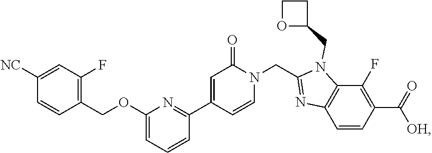 [SMILES: N#Cc1ccc(COc2cccc(-c3ccn(Cc4nc5ccc(C(=O)O)c(F)c5n4C[C@@H]4CCO4)c(=O)c3)n2)c(F)c1]

or a pharmaceutically acceptable salt thereof.

In one embodiment, the GLP-1 modulating compound is a compound disclosed in U.S. Pat. No. 9,474,755B2. In one embodiment, the GLP-1 modulating compound is

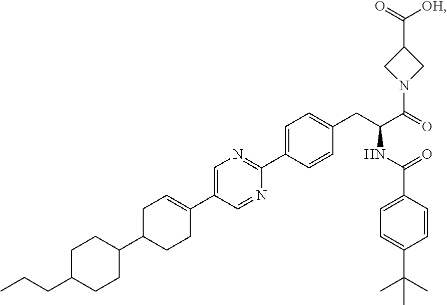 [SMILES: CCCC1CCC(C2CC=C(c3cnc(-c4ccc(C[C@H](NC(=O)c5ccc(C(C)(C)C)cc5)C(=O)N5CC(C(=O)O)C5)cc4)nc3)CC2)CC1]

or a pharmaceutically acceptable salt thereof.

In one embodiment, the GLP-1 modulating compound is a compound disclosed in CN115521297A. In one embodiment, the GLP-1 modulating compound is selected from the group consisting of

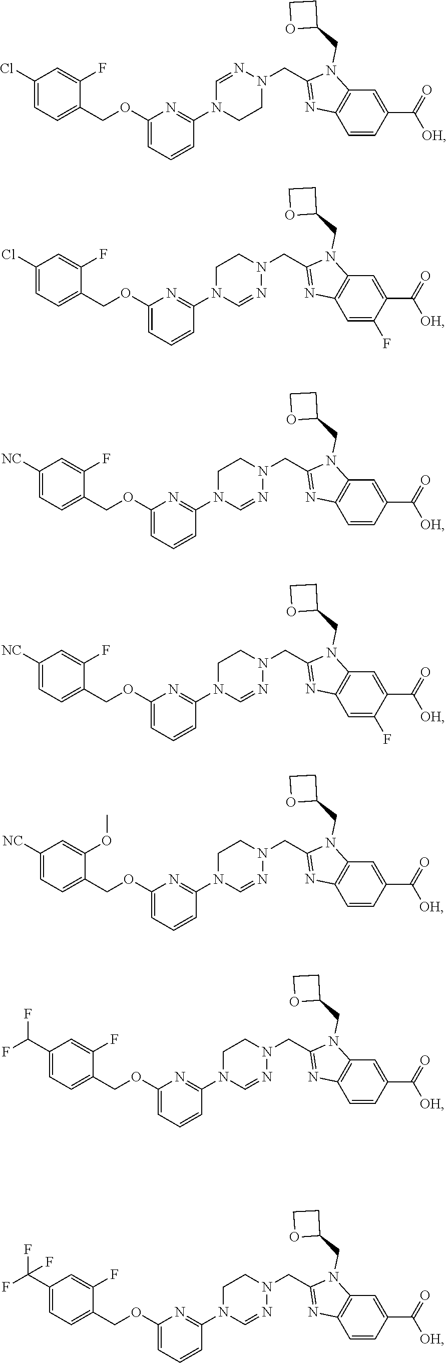

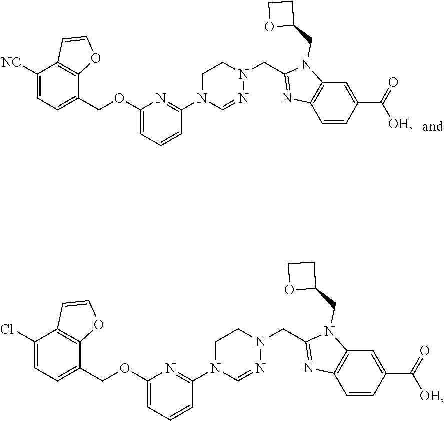 [SMILES: N#Cc1ccc(COc2cccc(N3C=NN(Cc4nc5ccc(C(=O)O)cc5n4C[C@@H]4CCO4)CC3)n2)c2occc12 ; O=C(O)c1ccc2nc(CN3CCN(c4cccc(OCc5ccc(Cl)c6ccoc56)n4)C=N3)n(C[C@@H]3CCO3)c2c1]

or a pharmaceutically acceptable salt thereof.

In one embodiment, the GLP-1 modulating compound is a compound disclosed in WO2023222124A1. In one embodiment, the GLP-1 modulating compound is

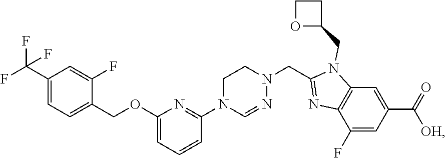 [SMILES: O=C(O)c1cc(F)c2nc(CN3CCN(c4cccc(OCc5ccc(C(F)(F)F)cc5F)n4)C=N3)n(C[C@@H]3CCO3)c2c1]

or a pharmaceutically acceptable salt thereof.

In one embodiment, the GLP-1 modulating compound is a compound disclosed in CN113227068B. In one embodiment, the GLP-1 modulating compound is

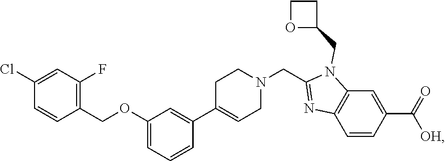 [SMILES: O=C(O)c1ccc2nc(CN3CC=C(c4cccc(OCc5ccc(Cl)cc5F)c4)CC3)n(C[C@@H]3CCO3)c2c1]

or a pharmaceutically acceptable salt thereof.

In one embodiment, the GLP-1 modulating compound is a compound disclosed in US20230365498A1. In one embodiment, the GLP-1 modulating compound is

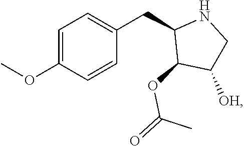 [SMILES: COc1ccc(C[C@H]2NC[C@H](O)[C@H]2OC(C)=O)cc1]

or a pharmaceutically acceptable salt thereof.

In one embodiment, the GLP-1 modulating compound is a compound disclosed in WO2023011395A1 or CN116217558A. In one embodiment, the GLP-1 modulating compound is selected from the group consisting of:

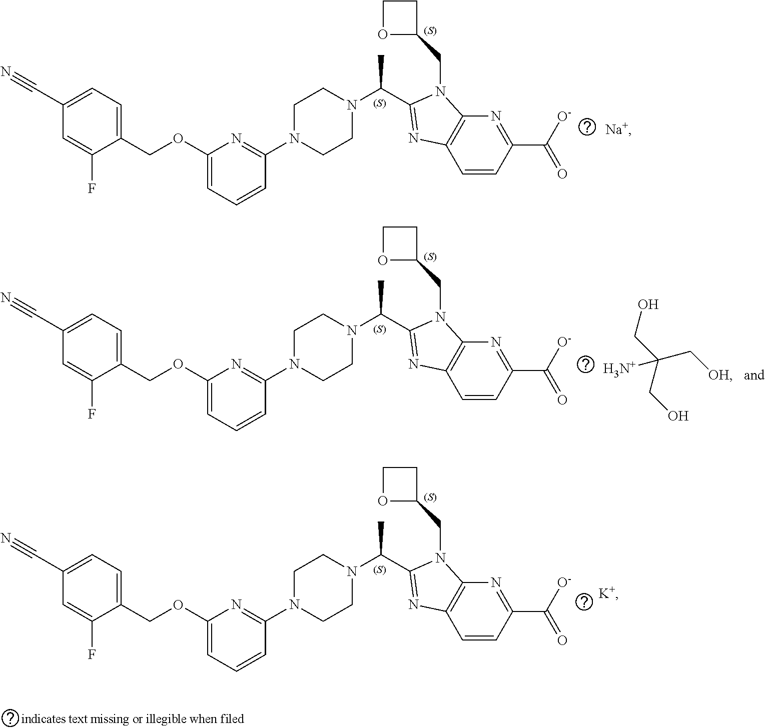

or a pharmaceutically acceptable salt thereof.

In one embodiment, the GLP-1 modulating compound is selected from the group consisting of

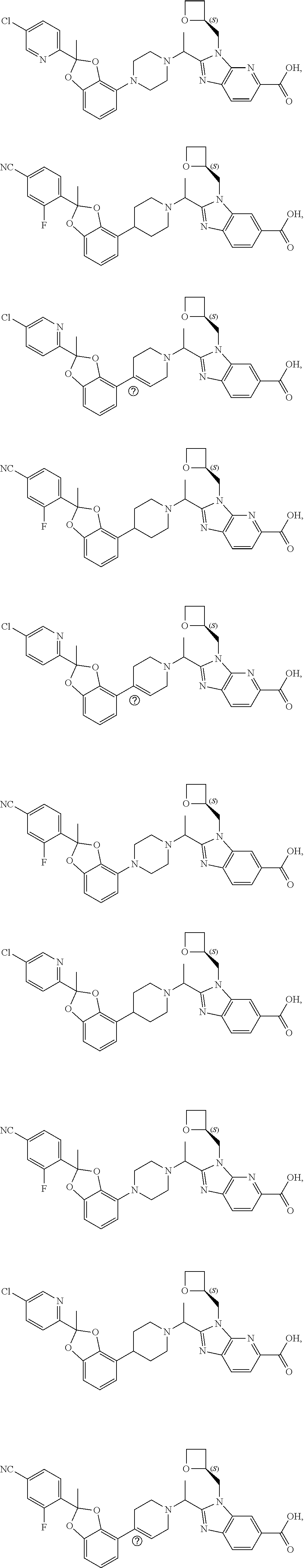

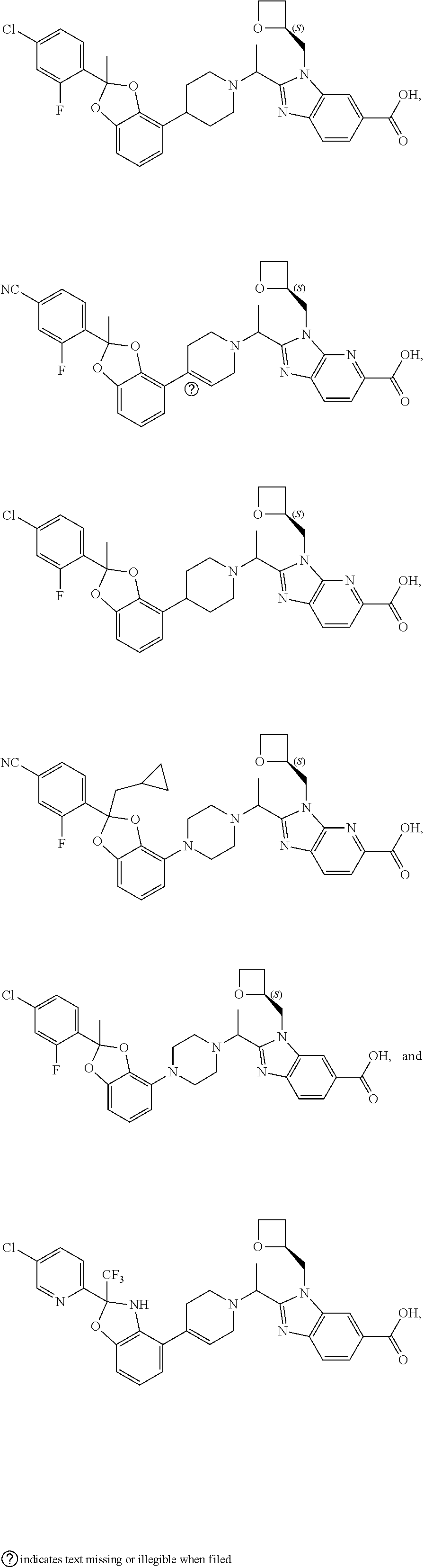

or a pharmaceutically acceptable salt thereof.

In one embodiment, the GLP-1 modulating compound is a compound disclosed in CN114591296A or CN114763352A. In one embodiment, the GLP-1 modulating compound is selected from the group consisting of

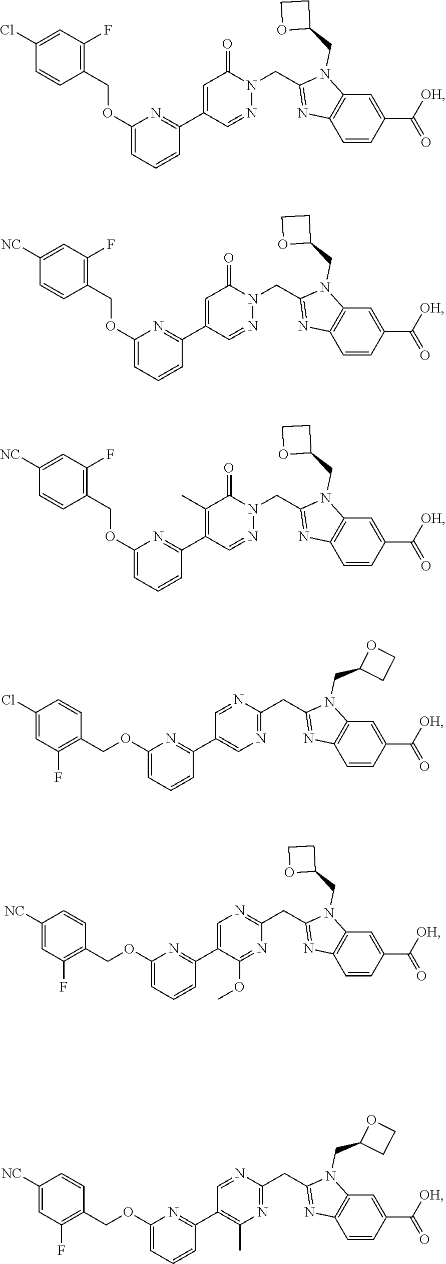 [SMILES: O=C(O)c1ccc2nc(Cn3ncc(-c4cccc(OCc5ccc(Cl)cc5F)n4)cc3=O)n(C[C@@H]3CCO3)c2c1 ; N#Cc1ccc(COc2cccc(-c3cnn(Cc4nc5ccc(C(=O)O)cc5n4C[C@@H]4CCO4)c(=O)c3)n2)c(F)c1 ; Cc1c(-c2cccc(OCc3ccc(C#N)cc3F)n2)cnn(Cc2nc3ccc(C(=O)O)cc3n2C[C@@H]2CCO2)c1=O ; O=C(O)c1ccc2nc(Cc3ncc(-c4cccc(OCc5ccc(Cl)cc5F)n4)cn3)n(C[C@@H]3CCO3)c2c1 ; COc1nc(Cc2nc3ccc(C(=O)O)cc3n2C[C@@H]2CCO2)ncc1-c1cccc(OCc2ccc(C#N)cc2F)n1 ; Cc1nc(Cc2nc3ccc(C(=O)O)cc3n2C[C@@H]2CCO2)ncc1-c1cccc(OCc2ccc(C#N)cc2F)n1]

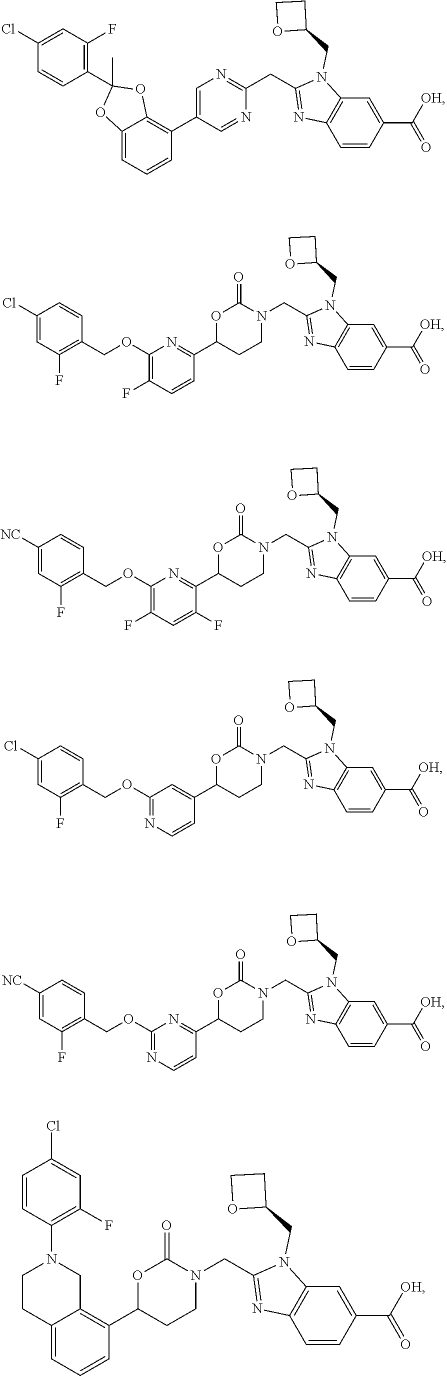 [SMILES: CC1(c2ccc(Cl)cc2F)Oc2cccc(-c3cnc(Cc4nc5ccc(C(=O)O)cc5n4C[C@@H]4CCO4)nc3)c2O1 ; O=C(O)c1ccc2nc(CN3CCC(c4ccc(F)c(OCc5ccc(Cl)cc5F)n4)OC3=O)n(C[C@@H]3CCO3)c2c1 ; N#Cc1ccc(COc2nc(C3CCN(Cc4nc5ccc(C(=O)O)cc5n4C[C@@H]4CCO4)C(=O)O3)c(F)cc2F)c(F)c1 ; O=C(O)c1ccc2nc(CN3CCC(c4ccnc(OCc5ccc(Cl)cc5F)c4)OC3=O)n(C[C@@H]3CCO3)c2c1 ; N#Cc1ccc(COc2nccc(C3CCN(Cc4nc5ccc(C(=O)O)cc5n4C[C@@H]4CCO4)C(=O)O3)n2)c(F)c1 ; O=C(O)c1ccc2nc(CN3CCC(c4cccc5c4CN(c4ccc(Cl)cc4F)CC5)OC3=O)n(C[C@@H]3CCO3)c2c1]

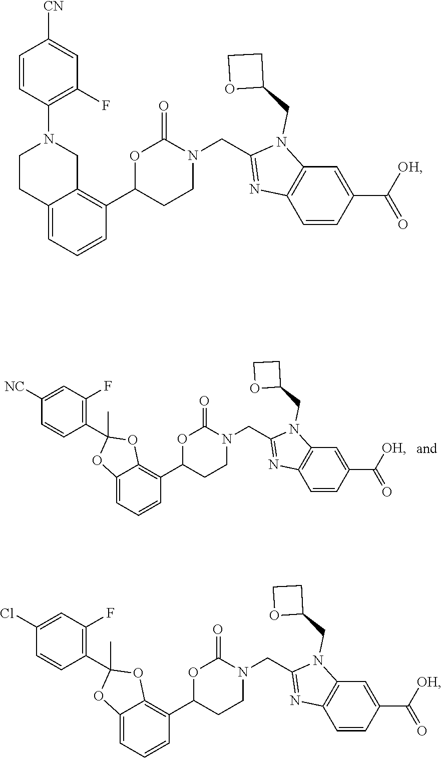 [SMILES: N#Cc1ccc(N2CCc3cccc(C4CCN(Cc5nc6ccc(C(=O)O)cc6n5C[C@@H]5CCO5)C(=O)O4)c3C2)c(F)c1 ; CC1(c2ccc(C#N)cc2F)Oc2cccc(C3CCN(Cc4nc5ccc(C(=O)O)cc5n4C[C@@H]4CCO4)C(=O)O3)c2O1 ; CC1(c2ccc(Cl)cc2F)Oc2cccc(C3CCN(Cc4nc5ccc(C(=O)O)cc5n4C[C@@H]4CCO4)C(=O)O3)c2O1]

or a pharmaceutically acceptable salt thereof.

In one embodiment, the GLP-1 modulating compound is a compound disclosed in WO2022116693A1. In one embodiment, the GLP-1 modulating compound is

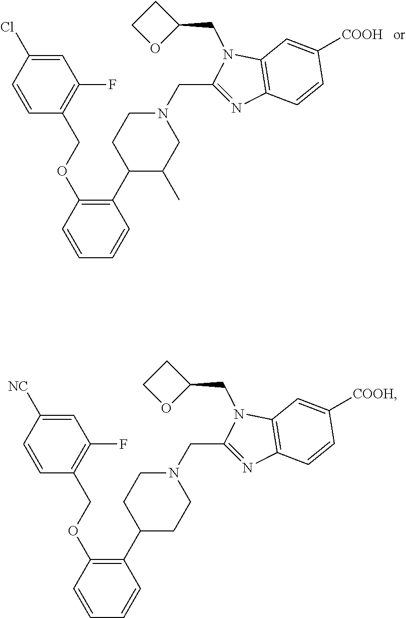 [SMILES: CC1CN(Cc2nc3ccc(C(=O)O)cc3n2C[C@@H]2CCO2)CCC1c1ccccc1OCc1ccc(Cl)cc1F ; "or" ; N#Cc1ccc(COc2ccccc2C2CCN(Cc3nc4ccc(C(=O)O)cc4n3C[C@@H]3CCO3)CC2)c(F)c1]

or a pharmaceutically acceptable salt thereof.

In one embodiment, the GLP-1 modulating compound is a compound disclosed in WO2023076237A1, WO2023049518A1, WO2023164050A1, TW202334129A, or TW202322806A. In one embodiment, the GLP-1 modulating compound is selected from the group consisting of

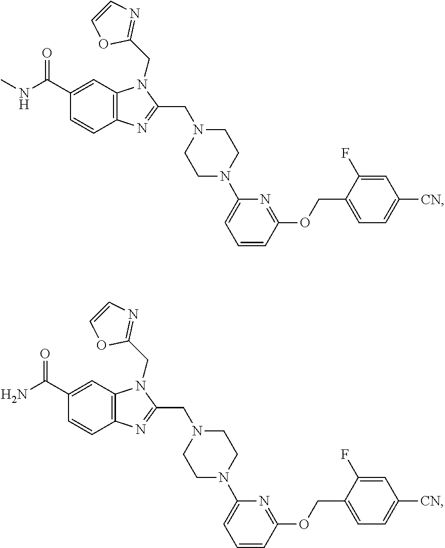 [SMILES: CNC(=O)c1ccc2nc(CN3CCN(c4cccc(OCc5ccc(C#N)cc5F)n4)CC3)n(Cc3ncco3)c2c1 ; N#Cc1ccc(COc2cccc(N3CCN(Cc4nc5ccc(C(N)=O)cc5n4Cc4ncco4)CC3)n2)c(F)c1]

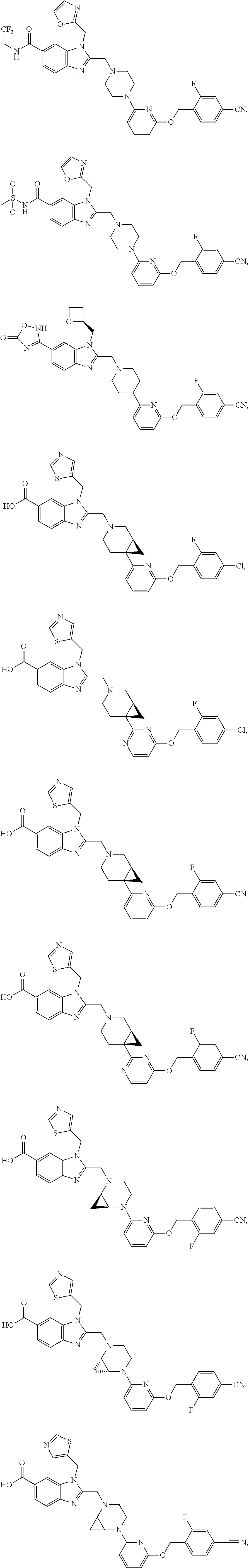

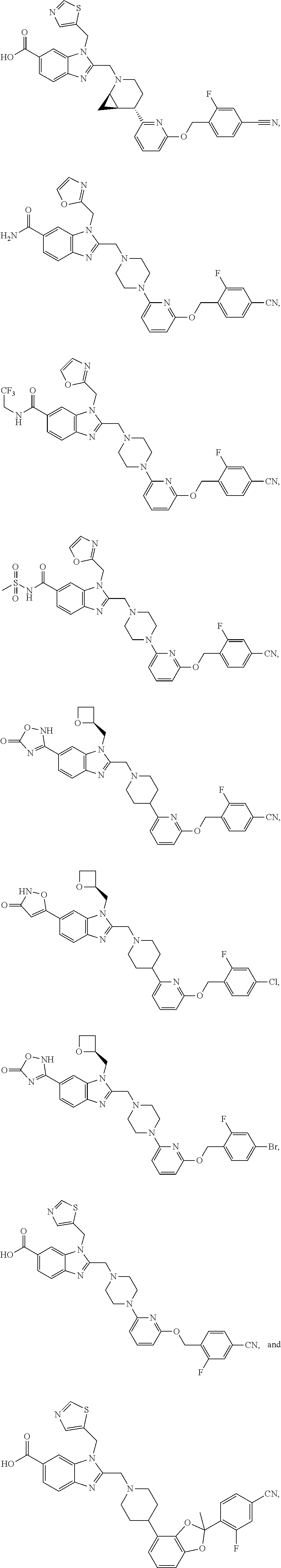

or a pharmaceutically acceptable salt thereof.

In one embodiment, the GLP-1 modulating compound is a compound disclosed in CN115703766A. In one embodiment, the GLP-1 modulating compound is

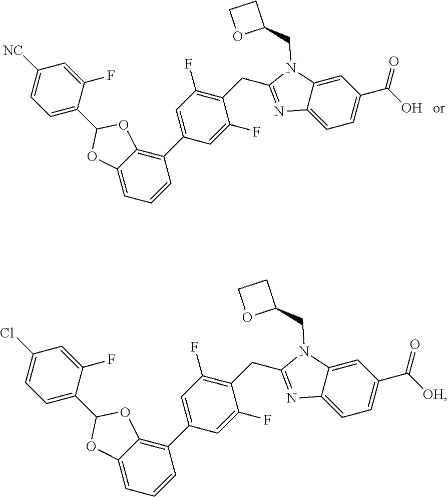 [SMILES: N#Cc1ccc(C2Oc3cccc(-c4cc(F)c(Cc5nc6ccc(C(=O)O)cc6n5C[C@@H]5CCO5)c(F)c4)c3O2)c(F)c1 ; O=C(O)c1ccc2nc(Cc3c(F)cc(-c4cccc5c4OC(c4ccc(Cl)cc4F)O5)cc3F)n(C[C@@H]3CCO3)c2c1]

or a pharmaceutically acceptable salt thereof.

In one embodiment, the GLP-1 modulating compound is a compound disclosed in WO2021018023A1. In one embodiment, the GLP-1 modulating compound is selected from the group consisting of

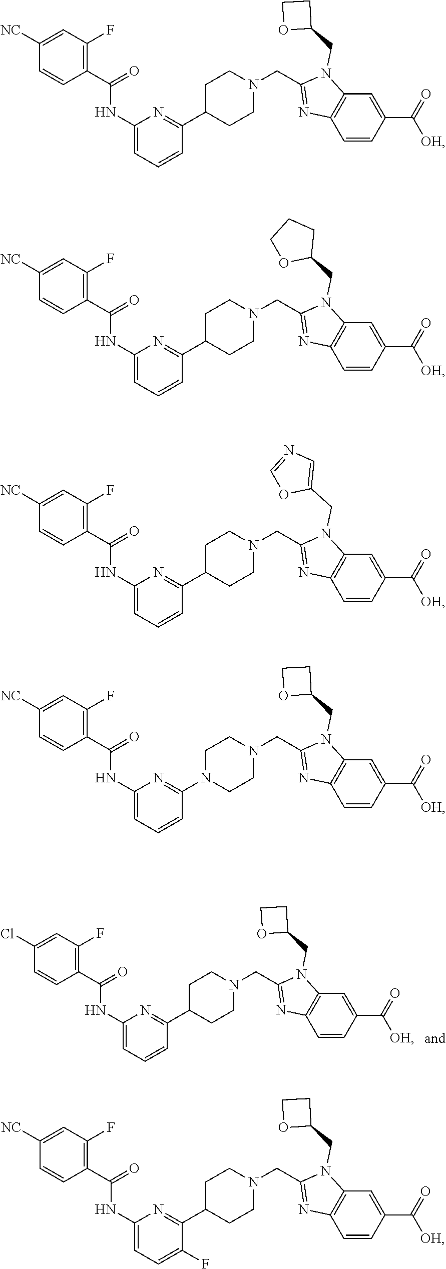 [SMILES: N#Cc1ccc(C(=O)Nc2cccc(C3CCN(Cc4nc5ccc(C(=O)O)cc5n4C[C@@H]4CCO4)CC3)n2)c(F)c1 ; N#Cc1ccc(C(=O)Nc2cccc(C3CCN(Cc4nc5ccc(C(=O)O)cc5n4C[C@@H]4CCCO4)CC3)n2)c(F)c1 ; N#Cc1ccc(C(=O)Nc2cccc(C3CCN(Cc4nc5ccc(C(=O)O)cc5n4Cc4cnco4)CC3)n2)c(F)c1 ; N#Cc1ccc(C(=O)Nc2cccc(N3CCN(Cc4nc5ccc(C(=O)O)cc5n4C[C@@H]4CCO4)CC3)n2)c(F)c1 ; O=C(O)c1ccc2nc(CN3CCC(c4cccc(NC(=O)c5ccc(Cl)cc5F)n4)CC3)n(C[C@@H]3CCO3)c2c1 ; N#Cc1ccc(C(=O)Nc2ccc(F)c(C3CCN(Cc4nc5ccc(C(=O)O)cc5n4C[C@@H]4CCO4)CC3)n2)c(F)c1]

or a pharmaceutically acceptable salt thereof.

In one embodiment, the GLP-1 modulating compound is a compound disclosed in WO2017117556A1. In one embodiment, the GLP-1 modulating compound is selected from the group consisting of

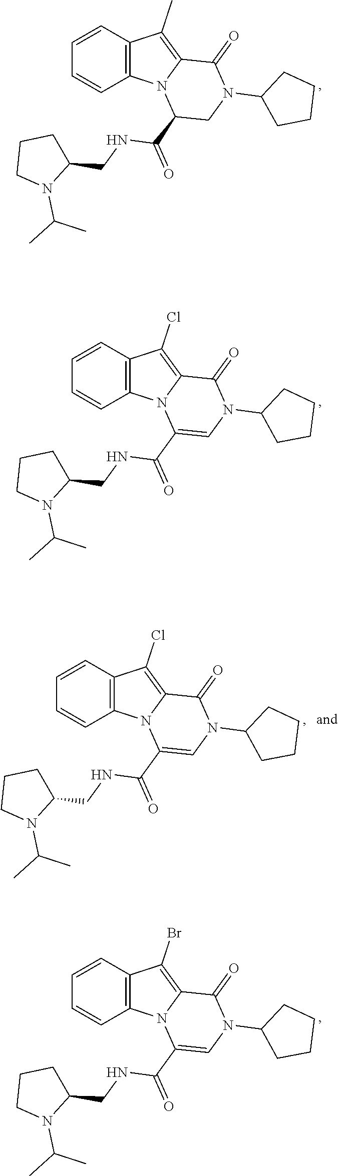 [SMILES: Cc1c2n(c3ccccc13)[C@H](C(=O)NC[C@@H]1CCCN1C(C)C)CN(C1CCCC1)C2=O ; CC(C)N1CCC[C@H]1CNC(=O)c1cn(C2CCCC2)c(=O)c2c(Cl)c3ccccc3n12 ; CC(C)N1CCC[C@@H]1CNC(=O)c1cn(C2CCCC2)c(=O)c2c(Cl)c3ccccc3n12 ; CC(C)N1CCC[C@H]1CNC(=O)c1cn(C2CCCC2)c(=O)c2c(Br)c3ccccc3n12]

or a pharmaceutically acceptable salt thereof.

In one embodiment, the GLP-1 modulating compound is a compound disclosed in WO2021238962A1. In one embodiment, the GLP-1 modulating compound is

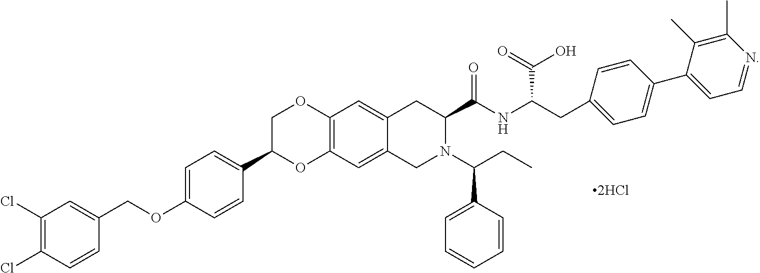 [SMILES: CC[C@@H](c1ccccc1)N1Cc2cc3c(cc2C[C@H]1C(=O)N[C@@H](Cc1ccc(-c2ccnc(C)c2C)cc1)C(=O)O)OC[C@H](c1ccc(OCc2ccc(Cl)c(Cl)c2)cc1)O3]

### Combination Therapy

The GIPR antagonist compounds of the disclosure may be used in combination with a GLP-1R agonist compound of the disclosure. The administration of a GIPR antagonist compound and a GLP-1R agonist compound “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “coadministration,” “simultaneous administration,” “sequential administration” and “administered simultaneously”.

The GIPR antagonist compounds and GLP-1R agonist compounds of the disclosure may be administered as a fixed or non-fixed combination of the active ingredients. The term “fixed combination” means a GIPR antagonist compound of the disclosure, or a pharmaceutically acceptable salt thereof, and a GLP-1R agonist compound of the disclosure, or a pharmaceutically acceptable salt thereof, are both administered to a subject simultaneously in a single composition or dosage. The term “non-fixed combination” means that a GIPR antagonist compound of the disclosure, or a pharmaceutically acceptable salt thereof, and a GLP-1R agonist compound of the disclosure, or a pharmaceutically acceptable salt thereof, are formulated as separate compositions or dosages such that they may be administered to a subject in need thereof simultaneously or at different times with variable intervening time limits, wherein such administration provides effective levels of the two or more compounds in the body of the subject.

In one embodiment, the present disclosure provides a first pharmaceutical composition comprising a GIPR antagonist compound of the disclosure or a pharmaceutically acceptable salt thereof, wherein the first pharmaceutical composition is administered in combination with a second pharmaceutical composition comprising a GLP-1R agonist compound of the disclosure or a pharmaceutically acceptable salt.

In some embodiments, the GIPR antagonist compound of the disclosure and the GLP-1R agonist compound of the disclosure are administered simultaneously. In some embodiments, the GIPR antagonist compound of the disclosure and the GLP-1R agonist compound of the disclosure are administered at sequentially. In some embodiments, a GIPR antagonist compound of the disclosure is administered to a subject first, and a GLP-1R agonist compound of the disclosure is administered to the subject second. In some embodiments, a GLP-1R agonist compound of the disclosure is administered to a subject first, and a GIPR antagonist compound of the disclosure is administered to the subject second.

These agents and compounds of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Administration and Dosing

Typically, each of the compounds used for combination therapy is administered in an amount effective to treat a disease or condition as described herein. A compound of the disclosure may be administered as compound per se, or alternatively, as a pharmaceutically acceptable salt. For administration and dosing purposes, a compound of the disclosure per se or pharmaceutically acceptable salt thereof will simply be referred to as a compound of the disclosure.

A compound of the disclosure can be administered by any suitable route in the form of a pharmaceutical composition adapted to such a route, and in a dose effective for the treatment intended. A compound of the disclosure may be administered orally, rectally, vaginally, parenterally, topically, intranasally, or by inhalation.

In some embodiments, a compound of the disclosure may be administered orally. Oral administration may involve swallowing, so that the compound enters the gastrointestinal tract, or buccal or sublingual administration may be employed by which the compound enters the bloodstream directly from the mouth.

In another embodiment, a compound of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, a compound of the disclosure may also be administered topically to the skin or mucosa, that is, dermally or transdermally. In another embodiment, a compound of the disclosure may also be administered intranasally or by inhalation. In another embodiment, a compound of the disclosure may be administered rectally or vaginally. In another embodiment, a compound of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. In another embodiment, total daily dose of the compound of the disclosure is from about 0.1 to about 50 mg/kg, and in another embodiment, from about 0.5 to about 30 mg/kg. It is not uncommon that the administration of the compounds of the disclosure will be repeated a plurality of times in a day (typically no greater than 4 times). Multiple doses per day typically may be used to increase the total daily dose, if desired.

In some embodiments, a GIPR antagonist compound of the disclosure is administered orally, and a GLP-1R compound of the disclosure is administered orally. In some embodiments, a GIPR antagonist compound of the disclosure is administered orally, and a GLP-1R compound of the disclosure is administered parenterally. In some embodiments, a GIPR antagonist compound of the disclosure is administered orally, and a GLP-1R compound of the disclosure is administered by subcutaneous injection. In some embodiments, a GIPR antagonist compound of the disclosure is administered parenterally, and a GLP-1R compound of the disclosure is administered orally. In some embodiments, a GIPR antagonist compound of the disclosure is administered by subcutaneous injection, and a GLP-1R compound of the disclosure is administered orally. In some embodiments, a GIPR antagonist compound of the disclosure is administered parenterally, and a GLP-1R compound of the disclosure is administered parenterally. In some embodiments, a GIPR antagonist compound of the disclosure is administered by subcutaneous injection, and a GLP-1R compound of the disclosure is administered by subcutaneous injection.

A GIPR antagonist compound of the disclosure or a pharmaceutically acceptable salt thereof can be administered in an amount of from about 1 mg to about 500 mg, 1 mg to about 400 mg, 1 mg to about 300 mg, 1 mg to about 200 mg, 1 mg to about 100 mg, 1 mg to about 50 mg, 1 mg to about 25 mg, or 1 mg to about 10 mg. In some embodiments, a GIPR antagonist compound of the disclosure or a pharmaceutically acceptable salt thereof can be administered in an amount of from about 1 mg to about 100 mg. In some embodiments, a GIPR antagonist compound of the disclosure or a pharmaceutically acceptable salt thereof can be administered in an amount of from about 1 mg to about 50 mg. In some embodiments, a GIPR antagonist compound of the disclosure or a pharmaceutically acceptable salt thereof can be administered in an amount of from about 1 mg to about 25 mg.

A GLP-1R agonist compound of the disclosure or a pharmaceutically acceptable salt thereof can be administered in an amount of from about 1 mg to about 500 mg, 1 mg to about 400 mg, 1 mg to about 300 mg, 1 mg to about 200 mg, 1 mg to about 100 mg, 1 mg to about 50 mg, 1 mg to about 25 mg, or 1 mg to about 10 mg. In some embodiments, a GLP-1R agonist compound of the disclosure or a pharmaceutically acceptable salt thereof can be administered in an amount of from about 1 mg to about 100 mg. In some embodiments, a GLP-1R agonist compound of the disclosure or a pharmaceutically acceptable salt thereof can be administered in an amount of from about 1 mg to about 50 mg. In some embodiments, a GLP-1R agonist compound of the disclosure or a pharmaceutically acceptable salt thereof can be administered in an amount of from about 1 mg to about 25 mg.

### Therapeutic Methods and Uses

The combination therapies described herein provide a method for modulating GIPR by contacting GIPR with a GIPR antagonist compound of the disclosure or a pharmaceutically acceptable salt thereof, and modulating GLP-1R by contacting GLP-1R with a GLP-1R agonist compound of the disclosure of a pharmaceutically acceptable salt thereof. The combination therapies disclosed herein may modulate GIPR, modulate GLP-1R, or modulate both GIPR and GLP-1R either in vitro or in vivo. The combination therapies disclosed herein may antagonize GIPR, agonize GLP-1R, or antagonize GIPR and agonize GLP-1R either in vitro or in vivo. In some embodiments, the combination therapy may modulate GIPR. In some embodiments, the combination therapy may antagonize GIPR. In some embodiments, the combination therapy may modulate GLP-1R. In some embodiments, the combination therapy may agonize GLP-1R. In some embodiments, the combination therapy may simultaneously modulate both GIPR and GLP-1R. In some embodiments, the combination therapy may simultaneously antagonize GIPR and agonize GLP-1R.

The combination therapies disclosed herein may be used to treat a disease or condition, disease, or disorder selected from the group consisting of: diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obstructive sleep apnea (OSA), obesity, an obesity-related comorbidity, sarcopenia resulting from chronic obesity, sexual dysfunction, an inflammatory disease, an eating disorder, nausea, emesis, weight loss, failure to thrive, sarcopenia, muscle wasting, muscle weakness, frailty, osteoporosis, a bone disorder, pain, neuropathic pain, anxiety, posttraumatic stress disorder (PTSD), depression, hypertension, malnutrition, weight gain, excessive sugar craving, dyslipidemia, hyperinsulinemia, nonalcoholic fatty liver disease (NAFLD), cardiovascular disease, atherosclerosis, coronary artery disease, peripheral vascular disease, hypertension, endothelial dysfunction, impaired vascular compliance, heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), cachexia, and addiction.

In some embodiments, the combination therapies disclosed herein may be used to treat diabetes, for example, Type 1 diabetes mellitus (T1D), Type 2 diabetes mellitus (T2DM), pre-diabetes, type 1b or idiopathic diabetes, latent autoimmune diabetes in adults (LADA), early-onset T2DM (EOD), youth-onset atypical diabetes (YOAD), maturity onset diabetes of the young (MODY), malnutrition-related diabetes, or gestational diabetes. In some embodiments, the combination therapies disclosed herein may be used to treat kidney disease, for example, acute kidney disorder, tubular dysfunction, proinflammatory changes to the proximal tubules, or chronic kidney disease (CKD). In some embodiments, the combination therapies disclosed herein may be used to treat obesity, for example, hypothalamic obesity or monogenic obesity. In some embodiments, the combination therapies disclosed herein may be used to treat an obesity-related comorbidity, for example, osteoarthritis or urine incontinence. In some embodiments, the combination therapies disclosed herein may be used to treat an eating disorder, for example, binge eating syndrome, bulimia nervosa, and syndromic obesity, such as Prader-Willi and Bardet-Biedl syndrome; or anorexia, anorexia nervosa, geriatric anorexia, anorexia associated with chemotherapy or radiotherapy.

In some embodiments, the combination therapies disclosed herein may be used to treat weight gain, for example, weight gain associated with the use of steroids or antipsychotics, weight gain caused by the treatment of depression, or weight gain caused by the use of agents that affect cognitive function. In some embodiments, the combination therapies disclosed herein may be used to treat dyslipidemia, for example, including hyperlipidemia, hypertriglyceridemia, increased total cholesterol, high LDL (low-density lipoprotein) cholesterol, and low HDL (high-density lipoprotein) cholesterol.

In some embodiments, the combination therapies disclosed herein may be used to treat NAFLD, for example, steatosis, nonalcoholic steatohepatitis (NASH), fibrosis, cirrhosis, and hepatocellular carcinoma. In some embodiments, the combination therapies disclosed herein may be used to treat heart failure, for example, congestive heart failure, heart failure with preserved ejection fraction (HFpEF), or heart failure with reduced ejection fraction (HFrEF). In some embodiments, the combination therapies disclosed herein may be used to treat myocardial infarction, for example, necrosis or apoptosis. In some embodiments, the combination therapies disclosed herein may be used to treat addiction, for example, addiction to alcohol, nicotine, or a drug substance. In some embodiments, the combination therapies disclosed herein may be used to treat cachexia, for example, cachexia associated with a chronic illness, cancer, acquired immunodeficiency syndrome (AIDS), heart failure, congestive heart failure (CHF), chronic kidney disease (CKD), treatment of a chronic illness such as cancer, heart failure, or CHF.

In some embodiments, the combination therapies disclosed herein may be used to treat obesity, for example, diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

### Kits

Another aspect of the disclosure provides kits comprising: 1) a GIPR antagonist compound of the disclosure or a pharmaceutically acceptable salt thereof, or a pharmaceutical composition comprising a GIPR antagonist compound of the disclosure or a pharmaceutically acceptable salt thereof; and 2) a GLP-1R agonist compound of the disclosure or a pharmaceutically acceptable salt thereof, or a pharmaceutical composition comprising a GLP-1R agonist compound of the disclosure or a pharmaceutically acceptable salt thereof.

A kit may include additional diagnostic or therapeutic agents. In some embodiments, a kit may also include instructions for use in a diagnostic or therapeutic method. In some embodiments, the kit includes) a GIPR antagonist compound of the disclosure or a pharmaceutically acceptable salt thereof, or a pharmaceutical composition comprising a GIPR antagonist compound of the disclosure or a pharmaceutically acceptable salt thereof; 2) a GLP-1R agonist compound of the disclosure or a pharmaceutically acceptable salt thereof, or a pharmaceutical composition comprising a GLP-1R agonist compound of the disclosure or a pharmaceutically acceptable salt thereof; and 3) a diagnostic agent.

In yet another embodiment, the disclosure comprises kits that are suitable for use in performing the methods of treatment described herein. In one embodiment, the kit contains: 1) a first dosage form comprising one or more GIPR antagonist compounds of the disclosure or a pharmaceutically acceptable salt thereof; and 2) a second dosage form comprising one or more GLP-1R agonist compounds of the disclosure or a pharmaceutically acceptable salt thereof, each in quantities sufficient to carry out the methods of the disclosure. In another embodiment, the kit contains: 1) a first dosage form comprising one or more GIPR antagonist compounds of the disclosure or a pharmaceutically acceptable salt thereof; 2) a second dosage form comprising one or more GLP-1R agonist compounds of the disclosure or a pharmaceutically acceptable salt thereof, each in quantities sufficient to carry out the methods of the disclosure; and 3) containers for one or more of the dosage forms.

### EXAMPLES

### Example 1. Functional In Vitro GIPR Antagonist Potency Assay

The functional in vitro antagonist potency for test compounds was determined by monitoring intracellular cyclic adenosine monophosphate (cAMP) levels in Chinese hamster ovary (CHO)—K1 cells stably expressing the human Glucose-dependent Insulinotropic Polypeptide Receptor (hGIPR). Following agonist activation, hGIPR associates with the G-protein complex causing the Gas subunit to exchange bound guanosine diphosphate (GDP) for guanosine triphosphate (GTP), followed by dissociation of the Gas-GTP complex. The activated Gas subunit can couple to downstream effectors to regulate the levels of second messengers or cAMP within the cell. Thereby, determination of intracellular cAMP levels allows for pharmacological characterization. Intracellular cAMP levels are quantitated using a homogenous assay utilizing the Homogeneous Time Resolved Fluorescence (HTRF) technology from Perkin Elmer. The method is a competitive immunoassay between native cAMP produced by the cells and cAMP labelled with the acceptor dye, d2. The two entities compete for binding to a monoclonal anti-cAMP antibody labeled with cryptate. The specific signal is inversely proportional to the concentration of cAMP in the cells.

Test compounds were solubilized to a concentration of 30 mM in 100% dimethyl sulfoxide (DMSO). An 11-point dilution series using 1 in 3.162-fold serial dilutions was created in 100% DMSO with a top concentration of 8 mM. The serially diluted compound was spotted with an Echo Acoustic liquid handler (Beckman Coulter) into a 384-well assay plate (Corning, Cat No. 3824) at 50 nL/well with duplicate points at each concentration, at a 200× final assay concentration (FAC). The final compound concentration range in the assay was 40 μM to 400 μM, with a final DMSO concentration of 0.5%.

Frozen assay-ready vials (at 1×10⁷ cells/vial) of CHO-K1 cells stably expressing the Gs-coupled human GIPR receptor (Eurofins, DiscoverX, Cat No. 95-0146C2) were thawed, counted, and resuspended in assay buffer consisting of Hank's Balanced Salt Solution (HBSS, Lonza Cat No. 10-527) containing 20 mM (4-(2-hydroxyethyl)-1-piperazineethanesulfonic acid (HEPES, Lonza, Cat No. 17-737E), 0.1% bovine serum albumin (BSA, Sigma, Cat No. A7979), and 200 μM 3-isobutyl-1-methylxanthine (IBMX, Sigma, Cat No. 15879) at a density of 4×10⁵ cells/mL. Cells were added to assay plate (5 μL/well of 4×10⁵ cells/mL stock for 2,000 cells/well final) containing 50 nL of 200×FAC test compound, and incubated at 37° C. (95% O₂: 5% CO₂) for 2 hours, with micro-clime lids (Labcyte, Cat No. LLS-0310). Following the 2-hour cell and compound incubation, a stimulation mix comprised of hGIPR agonist human glucose-dependent insulinotropic polypeptide (hGIP, full length, Sigma Cat No. G2269) in assay buffer/0.1% DMSO was added to the assay plate (5 μL/well) at an estimated EC₈₀ FAC (based on previous hGIP agonist curves) and incubated for another 30 minutes with micro-clime lids at 37° C. (95% O₂: 5% CO₂), after which intracellular cAMP levels were quantified as per Perkin Elmer's protocol (5 μL of d2 and then 5 μL cryptate, incubated for 1 hour at room temperature). Emission spectra of samples were measured on a Pherastar plate reader (BMG Labtech Inc) using a HTRF protocol (excitation, 320 nm; emission, 665 nm/620 nm).

hGIP EC₅₀ was determined daily by incubating cells (5 μL/well of 4×10⁵ cells/mL stock, for 2,000 cells/well final) with 50 nL 100% DMSO for 2 hours at 37° C. (95% O₂: 5% CO₂), with a micro-clime lid. Following the 2-hour cell and DMSO incubation, a hGIP concentration response curve at 2×FAC (12-point curve using 1 in 3 serial dilutions, with triplicate points at each concentration, 100 nM final top concentration) in assay buffer/1% DMSO was added (5 μL/well) and incubated for a further 30 minutes with a micro-clime lid at 37° C. (95% O₂: 5% CO₂), after which intracellular cAMP levels were quantified and samples measured as described previously. Experiments passed quality control if the agonist concentration used for stimulation fell between the on-the-day EC₅₀-EC₉₀.

Data were analyzed using the ratio of fluorescence intensity at 620 and 665 nm for each well, extrapolated from the cAMP standard curve to express data as nanomolar (nM) cAMP for each well. Data expressed as nM cAMP were then normalized to control wells using ActivityBase (IDBS data management software). Zero percent effect (ZPE) was defined as nM cAMP generated from the hGIP stimulation mix, while 100% effect, or one hundred percent effect (HPE), was defined as nM cAMP generated from the combined effects of hGIP simulation mix+antagonism by 80 μM of (-)-3-(6-(2-methyl-1-(4′-(trifluoromethyl)biphenyl-4-yl)propylamino)nicotinamido)propanoic acid as GIPR antagonist. The concentration and % effect values for each compound were plotted by ActivityBase using a four-parameter logistic dose response equation, and the concentration required for 50% inhibition (IC₅₀) was determined. Table 4 lists biological activities (IC₅₀ values) for Examples 1A-229A.

TABLE 4
Biological activity for Examples 1A-229A.

| | hGIPR antagonist | hGIPR antagonist IC₅₀ |
|---|---|---|
| Example | IC₅₀ (nM)¹ | replicate count |
| 1A | 16 | 12 |
| 2A | 74 | 3 |
| 3A | 17 | 6 |
| 4A | 4.1 | 14 |
| 5A | 9.1 | 6 |
| 6A | 6.5 | 5 |
| 7A | 8.1 | 5 |
| 8A | 150 | 5 |
| 9A | 130 | 3 |
| 10A | 67 | 3 |
| 11A | 180 | 3 |
| 12A | 10 | 5 |
| 13A | 12 | 9 |
| 14A | 88 | 3 |
| 15A | 8.1 | 6 |
| 16A | 3.7 | 7 |
| 17A | 14 | 9 |
| 18A | 15 | 9 |
| 19A | 11 | 10 |
| 20A | 12 | 14 |
| 21A | 11 | 19 |
| 22A | 62 | 10 |
| 23A | 2.7 | 5 |
| 24A | 110 | 4 |
| 25A | 86 | 4 |
| 26A | 2.1 | 4 |
| 27A | 4.8 | 18 |
| 28A | 10 | 16 |
| 29A | 17 | 14 |
| 30A | 5.7 | 10 |
| 31A | 7.8 | 10 |
| 32A | 6.1 | 3 |
| 33A | 6.3 | 6 |
| 34A | 10 | 5 |
| 35A | 4.6 | 4 |
| 36A | 46 | 3 |
| 37A | 58 | 3 |
| 38A | 17 | 3 |
| 39A | 3.7 | 3 |
| 40A | 7.3 | 7 |
| 41A | 160 | 3 |
| 42A | 15 | 5 |
| 43A | 49 | 3 |
| 44A | 6.8 | 4 |
| 45A | 540 | 5 |
| 46A | 160 | 3 |
| 47A | 47 | 3 |
| 48A | 23 | 3 |
| 49A | 14 | 4 |
| 50A | 83 | 2 |
| 51A | 280 | 4 |
| 52A | 120 | 3 |
| 53A | 15 | 1 |
| 54A | 140 | 4 |
| 55A | 7.6 | 3 |
| 56A | 6.3 | 7 |
| 57A | 65 | 3 |
| 58A | 50 | 3 |
| 59A | 35 | 3 |
| 60A | 21 | 4 |
| 61A | 150 | 4 |
| 62A | 110 | 5 |
| 63A | 6.2 | 5 |
| 64A | 2.9 | 10 |
| 65A | 13 | 4 |
| 66A | 390 | 54 |
| 67A | 14 | 3 |
| 68A | 38 | 3 |
| 69A | 47 | 2 |
| 70A | 49 | 1 |
| 71A | 2.5 | 3 |
| 72A | 69 | 1 |
| 73A | 12 | 4 |
| 74A | 82 | 4 |
| 75A | 140 | 4 |
| 76A | 9.9 | 7 |
| 77A | 16 | 7 |
| 78A | 145 | 4 |
| 79A | 15 | 3 |
| 80A | 400 | 4 |
| 81A | 210 | 4 |
| 82A | 27 | 3 |
| 83A | 230 | 3 |
| 84A | 19 | 5 |
| 85A | 260 | 4 |
| 86A | 7.5 | 4 |
| 87A | 11 | 3 |
| 88A | 430 | 5 |
| 89A | 18 | 3 |
| 90A | 45 | 5 |
| 91A | 39 | 2 |
| 92A | 87 | 3 |
| 93A | 79 | 2 |
| 94A | 190 | 3 |
| 95A | 13 | 3 |
| 96A | 180 | 4 |
| 97A | 230 | 3 |
| 98A | 30 | 3 |
| 99A | 18 | 3 |
| 100A | 27 | 3 |
| 101A | 92 | 3 |
| 102A | 690 | 5 |
| 103A | 29 | 3 |
| 104A | 2.9 | 4 |
| 105A | 8.8 | 7 |
| 106A | 3.0 | 3 |
| 107A | 720 | 6 |
| 108A | 260 | 4 |
| 109A | 5.1 | 3 |
| 110A | 3.7 | 5 |
| 111A | 11 | 4 |
| 112A | 840 | 4 |
| 113A | 21 | 4 |
| 114A | 40 | 5 |
| 115A | 4.8 | 4 |
| 116A | 12 | 3 |
| 117A | 26 | 3 |
| 118A | 15 | 3 |
| 119A | 24 | 3 |
| 120A | 26 | 4 |
| 121A | 27 | 4 |
| 122A | 99 | 4 |
| 123A | 11 | 4 |
| 124A | 74 | 3 |
| 125A | 19 | 6 |
| 126A | 6.6 | 4 |
| 127A | 69 | 3 |
| 128A | 9.3 | 3 |
| 129A | 14 | 4 |
| 130A | 11 | 4 |
| 131A | 140 | 3 |
| 132A | 77 | 4 |
| 133A | 180 | 4 |
| 134A | 140 | 4 |
| 135A | 52 | 3 |
| 136A | 7.8 | 3 |
| 137A | 9.8 | 3 |
| 138A | 42 | 3 |
| 139A | 93 | 3 |
| 140A | 67 | 3 |
| 141A | 97 | 3 |
| 142A | 130 | 3 |
| 143A | 35 | 1 |
| 144A | 6.1 | 7 |
| 145A | 70 | 3 |
| 146A | 9.7 | 4 |
| 147A | 13 | 3 |
| 148A | 240 | 3 |
| 149A | 21 | 5 |
| 150A | 86 | 3 |
| 151A | 110 | 3 |
| 152A | 44 | 3 |
| 153A | 11 | 5 |
| 154A | 58 | 3 |
| 155A | 22 | 4 |
| 156A | 45 | 4 |
| 157A | 10 | 7 |
| 158A | 7.7 | 6 |
| 159A | 110 | 4 |
| 160A | 75 | 3 |
| 161A | 77 | 3 |
| 162A | 44 | 3 |
| 163A | 19 | 5 |
| 164A | 22 | 4 |
| 165A | 4.6 | 3 |
| 166A | 5.7 | 3 |
| 167A | 7.2 | 7 |
| 168A | 15 | 5 |
| 169A | 49 | 3 |
| 170A | 40 | 3 |
| 171A | 16 | 4 |
| 172A | 31 | 4 |
| 173A | 4.4 | 3 |
| 174A | 49 | 5 |
| 175A | 8.3 | 4 |
| 176A | 11 | 3 |
| 177A | 8.2 | 5 |
| 178A | 11 | 4 |
| 179A | 3.7 | 3 |
| 180A | 53 | 4 |
| 181A | 24 | 4 |
| 182A | 23 | 4 |
| 183A | 9.3 | 5 |
| 184A | 43 | 3 |
| 185A | 4.2 | 4 |
| 186A | 36 | 3 |
| 187A | 51 | 4 |
| 188A | 32 | 3 |
| 189A | 42 | 3 |
| 190A | 9.9 | 3 |
| 191A | 14 | 3 |
| 192A | 12 | 3 |
| 193A | 17 | 3 |
| 194A | 14 | 4 |
| 195A | 35 | 6 |
| 196A | 49 | 3 |
| 197A | 29 | 3 |
| 198A | 69 | 3 |
| 199A | 71 | 2 |
| 200A | 9.9 | 4 |
| 201A | 91 | 3 |
| 202A | 22 | 3 |
| 203A | 8.4 | 4 |
| 204A | 14 | 4 |
| 205A | 14 | 3 |
| 206A | 14 | 4 |
| 207A | 1.3 | 5 |
| 208A | 4.9 | 5 |
| 209A | 7.4 | 4 |
| 210A | 9.3 | 3 |
| 211A | 7.0 | 4 |
| 212A | 18 | 3 |
| 213A | 90 | 3 |
| 214A | 6.7 | 4 |
| 215A | 15 | 3 |
| 216A | 65 | 3 |
| 217A | 140 | 2 |
| 218A | 33 | 3 |
| 219A | 2.9 | 3 |
| 220A | 47 | 3 |
| 221A | 11 | 4 |
| 222A | 100 | 3 |
| 223A | 51 | 3 |
| 224A | 79 | 5 |
| 225A | 51 | 3 |
| 226A | 81 | 3 |
| 227A | 53 | 3 |
| 228A | 36 | 3 |
| 229A | 11 | 3 |

### Example 2: CHO GLP-1R Clone Assays

CHO GLP-1R Clone C6 Assay (Assay 1): GLP-1R-mediated agonist activity was determined with a cell-based functional assay utilizing an HTRF (Homogeneous Time-Resolved Fluorescence) cAMP detection kit (cAMP HI Range Assay Kit; CisBio cat #62AM6PEJ) that measures cAMP levels in the cell. The method is a competitive immunoassay between native cAMP produced by the cells and exogenous cAMP labeled with the dye d2. The tracer binding is visualized by a mAb anti-cAMP labeled with Cryptate. The specific signal (i.e. energy transfer) is inversely proportional to the concentration of cAMP in either standard or experimental sample.

The human GLP-1R coding sequence (NCBI Reference Sequence NP_002053.3, including naturally-occurring variant Gly168Ser) was subcloned into pcDNA3 (Invitrogen) and a cell line stably expressing the receptor was isolated (designated Clone H6). Saturation binding analyses (filtration assay procedure) using ¹²⁵I-GLP-1₇₋₃₆ (Perkin Elmer) showed that plasma membranes derived from this cell line express a high GLP-1R density (K_{d}: 0.4 nM, Bₘₐₓ: 1900 fmol/mg protein).

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The cell pellet was then re-suspended in 10 mL of growth medium [DMEM/F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081) and 500 μg/mL Geneticin (G418) (Invitrogen #10131035)]. A 1 mL sample of the cell suspension in growth media was counted on a Becton Dickinson ViCell to determine cell viability and cell count per mL. The remaining cell suspension was then adjusted with growth media to deliver 2000 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment in 5% carbon dioxide.

Varying concentrations of each compound to be tested (in DMSO) were diluted in assay buffer (HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E) containing 100 μM 3-isobutyl-1-methylxanthin (IBMX; Sigma cat #15879). The final DMSO concentration is 1%.

After 48 hours, the growth media was removed from the assay plate wells, and the cells were treated with 20 μL of the serially diluted compound in assay buffer for 30 minutes at 37° C. in a humidified environment in 5% carbon dioxide. Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a cAMP standard curve (as described in the manufacturer's assay protocol) and the percent effect was determined relative to a saturating concentration of the full agonist GLP-1₇₋₃₆ (1 μM) included on each plate. EC₅₀ determinations were made from agonist dose-response curves analyzed with a curve fitting program using a 4-parameter logistic dose response equation.

CHO GLP-1R Clone C6 Assay (Assay 2): GLP-1R-mediated agonist activity was determined with a cell-based functional assay utilizing an HTRF (Homogeneous Time-Resolved Fluorescence) cAMP detection kit (cAMP HI Range Assay Kit; Cis Bio cat #62AM6PEJ) that measures cAMP levels in the cell. The method is a competitive immunoassay between native cAMP produced by the cells and exogenous cAMP labeled with the dye d2. The tracer binding is visualized by a mAb anti-cAMP labeled with Cryptate. The specific signal (i.e. energy transfer) is inversely proportional to the concentration of cAMP in either a standard or an experimental sample.

The human GLP-1R coding sequence (NCBI Reference Sequence NP_002053.3, including naturally-occurring variant Leu260Phe) was subcloned into pcDNA5-FRT-TO and a clonal CHO cell line stably expressing a low receptor density was isolated using the Flp-In™ T-Rex™ System, as described by the manufacturer (ThermoFisher). Saturation binding analyses (filtration assay procedure) using ¹²⁵I-GLP-1 (Perkin Elmer) showed that plasma membranes derived from this cell line (designated clone C6) express a low GLP-1R density (K_{d}: 0.3 nM, Bₘₐₓ: 240 fmol/mg protein), relative to the clone H6 cell line.

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The DPBS was aspirated, and the cell pellet was re-suspended in 10 mL of complete growth medium (DMEM:F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081), 700 μg/mL Hygromycin (Invitrogen Cat #10687010) and 15 μg/mL Blasticidin (Gibco Cat #R21001). A 1 mL sample of the cell suspension in growth media was counted on a Becton Dickinson ViCell to determine cell viability and cell count per mL. The remaining cell suspension was then adjusted with growth media to deliver 1600 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment (95% O₂, 5% CO₂) Varying concentrations of each compound to be tested (in DMSO) were diluted in assay buffer [HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E)] containing 100 μM 3-isobutyl-1-methylxanthin (IBMX; Sigma cat #15879). The final DMSO concentration in the compound/assay buffer mixture is 1%.

After 48 hours, the growth media was removed from the assay plate wells, and the cells were treated with 20 μL of the serially diluted compound in assay buffer for 30 minutes at 37° C. in a humidified environment (95% O₂, 5% CO₂). Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a cAMP standard curve (as described in the manufacturer's assay protocol) and the percent effect was determined relative to a saturating concentration of the full agonist GLP-1 (1 μM) included on each plate. EC₅₀ determinations were made from agonist dose response curves analyzed with a curve fitting program using a 4-parameter logistic dose response equation.

In Table 5 and Table 6, assay data are presented to two (2) significant figures as the geometric mean (EC₅₀s) and arithmetic mean (Emax) based on the number of replicates listed (Number). A blank cell means there was no data for that Example or the Emax was not calculated.

TABLE 5
Biological Activity of Examples 1B-106B

| | Assay 1 | Assay 1 | | Assay 2 | Assay 2 | |
|---|---|---|---|---|---|---|
| | EC₅₀ | Emax | Assay 1 | EC₅₀ | Emax | Assay 2 |
| Ex. No. | (nM) | (%) | Number | (nM) | (%) | Number |
| 1B | 880 | 99 | 3 | >20000 | | 1 |
| 2B* | 6.6 | 81 | 5 | 260 | 100 | 4 |
| 3B | 1.3 | 94 | 3 | 45 | 120 | 3 |
| 4B | 1600 | 87 | 3 | >20000 | | 1 |
| 5B** | 1.3 | 89 | 6 | 23 | 97 | 7 |
| 7B | 140 | 89 | 7 | 2400 | 89 | 5 |
| 8B** | 0.26 | 98 | 3 | 3.1 | 93 | 12 |
| 11B*** | 0.30 | 92 | 6 | 3.6 | 91 | 6 |
| 12B*** | 73 | 88 | 9 | 1600 | 90 | 4 |
| 13B**** | 0.96 | 99 | 5 | 17 | 96 | 8 |
| 15B | 290 | 78 | 3 | | | |
| 16B | 29 | 83 | 3 | 690 | 92 | 3 |
| 17B | 4.5 | 95 | 3 | 38 | 110 | 3 |
| 18B | 7 | 95 | 6 | 79 | 85 | 5 |
| 19B | >18000 | 100 | 3 | >20000 | | 1 |
| 20B | 7.7 | 90 | 3 | 120 | 64 | 3 |
| 21B | 0.079 | 97 | 3 | 1.1 | 96 | 4 |
| 22B | 210 | 97 | 3 | 1000 | 87 | 3 |
| 23B | 1.2 | 87 | 3 | 25 | 100 | 3 |
| 24B | 17 | 85 | 3 | 270 | 100 | 3 |
| 25B | >20000 | | 1 | >20000 | | 1 |
| 26B | >20000 | | 1 | | | |
| 27B | 680 | 76 | 3 | | | |
| 28B | 1.4 | 82 | 3 | 49 | 110 | 3 |
| 29B | >20000 | | 1 | >20000 | | 1 |
| 30B | >20000 | | 1 | >20000 | | 1 |
| 31B | 61 | 98 | 3 | 1000 | 100 | 3 |
| 32B | 480 | 87 | 3 | | | |
| 33B | 5.3 | 87 | 4 | 150 | 93 | 3 |
| 34B | 45 | 86 | 4 | 1100 | 77 | 4 |
| 35B | 190 | 88 | 3 | 1900 | 65 | 3 |
| 36B | 18 | 86 | 3 | 450 | 87 | 3 |
| 37B | 2.6 | 85 | 3 | 100 | 86 | 3 |
| 38B | 7.8 | 98 | 3 | 110 | 88 | 3 |
| 39B | 6.6 | 86 | 3 | 170 | 89 | 3 |
| 40B | 760 | 85 | 3 | | | |
| 41B | 81 | 100 | 3 | 1000 | 83 | 3 |
| 42B | 10 | 87 | 3 | 240 | 73 | 3 |
| 43B | 200 | 83 | 3 | | | |
| 44B | 14 | 88 | 3 | 130 | 73 | 3 |
| 45B | 91 | 78 | 3 | 2000 | 74 | 2 |
| 46B | 120 | 93 | 3 | 1700 | 83 | 3 |
| 47B | 3.5 | 88 | 4 | 65 | 86 | 3 |
| 48B | 160 | 78 | 4 | | | |
| 49B | 9.9 | 81 | 3 | 220 | 79 | 3 |
| 50B | 5.2 | 95 | 4 | 57 | 96 | 3 |
| 51B | 42 | 75 | 3 | 1400 | 76 | 4 |
| 52B | 14 | 81 | 3 | 280 | 73 | 3 |
| 53B | 230 | 93 | 3 | | | |
| 54B | 12 | 87 | 4 | 140 | 92 | 4 |
| 55B | 19 | 80 | 3 | 280 | 81 | 3 |
| 56B | 32 | 85 | 3 | 570 | 80 | 3 |
| 57B | 3.1 | 87 | 3 | 52 | 84 | 4 |
| 58B | 18 | 82 | 3 | 160 | 64 | 3 |
| 59B | 74 | 81 | 3 | 1100 | 50 | 3 |
| 60B | 1.2 | 87 | 4 | 11 | 81 | 3 |
| 61B | 15 | 86 | 3 | 500 | 98 | 3 |
| 62B | 4 | 98 | 3 | 23 | 88 | 4 |
| 63B | 74 | 85 | 3 | 680 | 53 | 3 |
| 64B | 15 | 82 | 3 | 240 | 60 | 3 |
| 65B | 10 | 79 | 3 | 240 | 85 | 3 |
| 66B | 2.2 | 94 | 3 | 82 | 95 | 3 |
| 67B | 5.2 | 91 | 3 | 66 | 96 | 3 |
| 68B | 9.2 | 94 | 3 | 91 | 80 | 3 |
| 69B | 1.2 | 99 | 3 | 11 | 99 | 6 |
| 70B | 51 | 82 | 3 | 850 | 74 | 3 |
| 71B | 710 | 83 | 3 | | | |
| 72B | 73 | 89 | 3 | 1200 | 94 | 3 |
| 73B | 10 | 100 | 3 | 8.3 | 98 | 3 |
| 74B | 2.8 | 100 | 4 | 97 | 100 | 4 |
| 75B | 6.8 | 80 | 4 | 74 | 80 | 3 |
| 76B | 14 | 76 | 3 | 310 | 80 | 3 |
| 77B | 1.7 | 98 | 3 | 10 | 100 | 3 |
| 78B | 460 | 90 | 3 | | | |
| 79B | 65 | 82 | 3 | 1000 | 71 | 3 |
| 80B | 0.77 | 93 | 3 | 7.6 | 100 | 3 |
| 81B | 53 | 89 | 3 | 1700 | 92 | 3 |
| 82B | 4.5 | 89 | 4 | 78 | 100 | 3 |
| 83B | 1.4 | 85 | 3 | 21 | 85 | 3 |
| 84B | 1.1 | 87 | 3 | 6.9 | 96 | 4 |
| 85B | 29 | 110 | 3 | 54 | 110 | 3 |
| 86B | 47 | 83 | 3 | 1000 | 83 | 3 |
| 87B | 3.4 | 85 | 4 | 44 | 88 | 4 |
| 88B | 9.1 | 93 | 3 | 100 | 86 | 3 |
| 89B | 230 | 80 | 3 | | | |
| 90B | 24 | 91 | 3 | 410 | 100 | 3 |
| 91B | 570 | 89 | 3 | | | |
| 92B | 17 | 86 | 3 | 360 | 91 | 3 |
| 93B | 130 | 85 | 3 | 2900 | 87 | 3 |
| 94B | >20000 | | 1 | | | |
| 95B | 14000 | 100 | 3 | >20000 | | 1 |
| 96B | 4.2 | 90 | 5 | 72 | 83 | 3 |
| 97B | >6500 | 84 | 5 | >20000 | | 1 |
| 98B | 12 | 89 | 5 | 360 | 87 | 3 |
| 99B | 220 | 77 | 3 | >13000 | | 5 |
| 100B | 1.1 | 85 | 3 | 11 | 93 | 4 |
| 101B | 14 | 86 | 3 | 140 | 93 | 4 |
| 102B | 50 | 97 | 3 | 440 | 95 | 3 |
| 103B | 2.8 | 99 | 4 | 5.4 | 91 | 2 |
| 104B | | | | 7.6 | 99 | 1 |
| 105B | | | | 19 | 74 | 1 |
| 106B | 600 | 86 | 4 | | | |

*Tested as ammonium and trifluoroacetate salts
**Tested as ammonium and 1,3-dihydroxy-2-(hydroxymethyl)propan-2-aminium (Tris) salts, and free acid
***Tested as ammonium salt and free acid
****Tested as formate salt and free acid

TABLE 6
Biological Activity of Examples 107B-364B

| Example | Assay 1 | Assay 1 | Assay 1 | Assay 2 | Assay 2 | Assay 2 |
|---|---|---|---|---|---|---|
| number | EC₅₀ (nM) | Emax (%) | Number | EC₅₀ (nM) | Emax (%) | Number |
| 107B | 81 | 77 | 5 | 910 | 94 | 4 |
| 108B | 16 | 85 | 6 | 320 | 88 | 11 |
| 109B | 4.3 | 83 | 3 | 92 | 83 | 3 |
| 110B | 21 | 79 | 3 | 350 | 82 | 3 |
| 111B | 42 | 75 | 3 | 530 | 67 | 3 |
| 112B | 29 | 84 | 3 | 350 | 70 | 3 |
| 113B | 3.9 | 82 | 4 | 45 | 82 | 5 |
| 114B | 7.1 | 81 | 3 | 120 | 85 | 3 |
| 115B | 0.95 | 92 | 1 | 17 | 120 | 3 |
| 116B | 930 | 86 | 3 | 9000 | 100 | 3 |
| 117B | 19 | 76 | 3 | 530 | 97 | 3 |
| 118B | 750 | 76 | 3 | | | |
| 119B | 210 | 66 | 3 | | | |
| 120B | 47 | 71 | 3 | 1600 | 81 | 3 |
| 121B | >20000 | | 1 | | | |
| 122B | 1.5 | 86 | 3 | 14 | 82 | 3 |
| 123B | 2.4 | 87 | 3 | 45 | 95 | 3 |
| 124B | 6.4 | 90 | 4 | 110 | 94 | 3 |
| 125B | 0.28 | 84 | 5 | 3.2 | 84 | 4 |
| 126B | 44 | 81 | 3 | 880 | 90 | 3 |
| 127B | 4.9 | 77 | 7 | 75 | 83 | 3 |
| 128B | 36 | 78 | 3 | 400 | 86 | 3 |
| 129B | 50 | 83 | 3 | 1000 | 98 | 3 |
| 130B | >11000 | 84 | 3 | | | |
| 131B | 45 | 65 | 3 | 1000 | 84 | 3 |
| 132B | 70 | 80 | 3 | 1800 | 85 | 3 |
| 133B | 190 | 91 | 3 | 1400 | 47 | 3 |
| 134B | 300 | 92 | 3 | 7700 | 100 | 3 |
| 135B | 260 | 88 | 3 | 5600 | 86 | 3 |
| 136B | 150 | 86 | 3 | 4100 | 110 | 3 |
| 137B | 90 | 76 | 55 | 1800 | 89 | 43 |
| 138B | 7.9 | 95 | 5 | 110 | 89 | 7 |
| 139B | 150 | 74 | 3 | 1200 | 44 | 5 |
| 140B | 36 | 84 | 6 | 150 | 92 | 3 |
| 141B | 6 | 73 | 3 | 150 | 84 | 3 |
| 142B | 9 | 82 | 3 | 170 | 85 | 3 |
| 143B | 140 | 72 | 3 | 1600 | 46 | 3 |
| 144B | 5.8 | 72 | 3 | 94 | 76 | 4 |
| 145B | 120 | 76 | 4 | 2200 | 83 | 3 |
| 146B | 43 | 82 | 3 | 680 | 110 | 4 |
| 147B | 210 | 79 | 3 | | | |
| 148B | 340 | 74 | 3 | | | |
| 149B | 770 | 78 | 3 | >15000 | 100 | 3 |
| 150B | 110 | 71 | 3 | 3000 | 92 | 3 |
| 151B | 22 | 87 | 3 | 280 | 91 | 3 |
| 152B | 370 | 75 | 3 | | | |
| 153B | 24 | 76 | 5 | 1200 | 94 | 8 |
| 154B | 6 | 80 | 3 | 78 | 88 | 3 |
| 155B | 7.9 | 80 | 5 | 150 | 100 | 3 |
| 156B | 0.96 | 80 | 5 | 14 | 95 | 2 |
| 157B | 8.5 | 88 | 3 | 220 | 110 | 3 |
| 158B | 14 | 72 | 4 | 190 | 83 | 3 |
| 159B | 0.5 | 73 | 3 | 22 | 96 | 6 |
| 160B | 0.94 | 83 | 15 | 12 | 98 | 21 |
| 161B | 1.1 | 79 | 5 | 13 | 100 | 18 |
| 162B | 0.34 | 78 | 6 | 7.4 | 94 | 6 |
| 163B | 0.76 | 88 | 9 | 8.8 | 100 | 15 |
| 164B | 3.2 | 84 | 7 | 55 | 86 | 11 |
| 165B | 100 | 99 | 3 | 1100 | 90 | 3 |
| 166B | 150 | 80 | 3 | | | |
| 167B | 18 | 84 | 3 | 350 | 77 | 3 |
| 168B | 120 | 79 | 3 | 1700 | 88 | 3 |
| 169B | 1200 | 78 | 3 | | | |
| 170B | 79 | 81 | 3 | 4000 | 75 | 3 |
| 171B | 200 | 79 | 3 | | | |
| 172B | 2900 | 89 | 3 | | | |
| 173B | 2700 | 85 | 3 | | | |
| 174B | 870 | 82 | 3 | | | |
| 175B | 170 | 78 | 8 | 2100 | 71 | 3 |
| 176B | 2400 | 87 | 3 | | | |
| 177B | 880 | 59 | 3 | | | |
| 178B | 4500 | 48 | 4 | | | |
| 179B | 1600 | 83 | 3 | | | |
| 180B | >17000 | 100 | 3 | | | |
| 181B | 170 | 68 | 3 | | | |
| 182B | 1800 | 93 | 3 | | | |
| 183B | 180 | 78 | 8 | 3500 | 90 | 3 |
| 184B | 3.2 | 77 | 3 | 43 | 84 | 3 |
| 185B | 0.34 | 89 | 5 | 2.2 | 97 | 5 |
| 186B | 1700 | 82 | 3 | | | |
| 187B | 49 | 71 | 3 | 2100 | 90 | 3 |
| 188B | 2.4 | 71 | 4 | 38 | 83 | 3 |
| 189B | 68 | 77 | 3 | 1700 | 110 | 3 |
| 190B | 2.8 | 82 | 3 | 68 | 81 | 3 |
| 191B | 0.55 | 77 | 4 | 9.2 | 94 | 6 |
| 192B | 0.99 | 95 | 4 | 10 | 120 | 7 |
| 193B | 0.3 | 84 | 4 | 4.1 | 100 | 2 |
| 194B | 75 | 69 | 3 | | | |
| 195B | 110 | 68 | 3 | 4400 | 110 | 3 |
| 196B | 4.1 | 67 | 3 | 170 | 75 | 3 |
| 197B | 8.1 | 74 | 3 | 220 | 93 | 4 |
| 198B | 1.3 | 72 | 4 | 27 | 87 | 3 |
| 199B | 1.7 | 67 | 3 | 45 | 83 | 3 |
| 200B | 1.8 | 77 | 3 | 59 | 85 | 3 |
| 201B | 1.6 | 78 | 7 | 33 | 78 | 3 |
| 202B | >19000 | 100 | 3 | | | |
| 203B | >15000 | 98 | 3 | >20000 | | 1 |
| 204B | 18 | 88 | 3 | 400 | 100 | 3 |
| 205B | 5.7 | 67 | 3 | 100 | 79 | 3 |
| 206B | 38 | 76 | 3 | 760 | 91 | 3 |
| 207B | 5.7 | 78 | 3 | 130 | 94 | 3 |
| 208B | 1.7 | 82 | 3 | 41 | 99 | 3 |
| 209B | 7 | 59 | 3 | 190 | 80 | 3 |
| 210B | 13 | 75 | 3 | 250 | 80 | 3 |
| 211B | 50 | 81 | 3 | 870 | 83 | 3 |
| 212B | 190 | 71 | 3 | | | |
| 213B | 390 | 82 | 3 | | | |
| 214B | 550 | 71 | 3 | | | |
| 215B | 3.4 | 73 | 3 | 59 | 89 | 3 |
| 216B | 0.47 | 77 | 5 | 5 | 94 | 6 |
| 217B | 1.5 | 73 | 3 | 24 | 91 | 3 |
| 218B | 1.5 | 80 | 7 | 29 | 95 | 5 |
| 219B | 0.3 | 93 | 5 | 3.8 | 93 | 6 |
| 220B | 0.33 | 82 | 3 | 13 | 110 | 7 |
| 221B | 3.4 | 86 | 6 | 23 | 110 | 7 |
| 222B | 130 | 72 | 3 | | | |
| 223B | 170 | 69 | 3 | | | |
| 224B | 1200 | 53 | 3 | | | |
| 225B | 12 | 75 | 4 | 140 | 82 | 3 |
| 226B | 2.7 | 73 | 4 | 29 | 74 | 5 |
| 227B | 4 | 64 | 3 | 48 | 79 | 3 |
| 228B | 6 | 73 | 3 | 99 | 81 | 3 |
| 229B | 1 | 76 | 3 | 19 | 75 | 3 |
| 230B | 1700 | 80 | 3 | | | |
| 231B | 0.3 | 80 | 3 | 4.4 | 95 | 6 |
| 232B | 8.6 | 64 | 3 | 140 | 76 | 3 |
| 233B | 0.56 | 73 | 6 | 9.6 | 84 | 3 |
| 234B | 290 | 64 | 3 | | | |
| 235B | 390 | 72 | 3 | | | |
| 236B | 1300 | 79 | 3 | | | |
| 237B | 3.2 | 68 | 3 | 39 | 88 | 3 |
| 238B | 0.71 | 81 | 5 | 11 | 100 | 6 |
| 239B | 1.2 | 84 | 5 | 24 | 98 | 5 |
| 240B | 0.5 | 78 | 3 | 16 | 86 | 5 |
| 241B | 9.4 | 69 | 3 | 160 | 71 | 3 |
| 242B | 0.52 | 70 | 4 | 8.5 | 78 | 4 |
| 243B | 4.7 | 71 | 3 | 84 | 83 | 4 |
| 244B | 31 | 81 | 3 | 460 | 66 | 3 |
| 245B | 1.5 | 74 | 6 | 35 | 89 | 3 |
| 246B | 2.9 | 96 | 4 | 28 | 97 | 3 |
| 247B | 0.58 | 86 | 3 | 11 | 110 | 5 |
| 248B | 2 | 82 | 3 | 32 | 79 | 3 |
| 249B | 1.6 | 84 | 6 | 35 | 78 | 3 |
| 250B | 0.39 | 84 | 4 | 3.4 | 100 | 4 |
| 251B | 0.59 | 83 | 5 | 6.8 | 93 | 5 |
| 252B | 140 | 76 | 4 | | | |
| 253B | 2 | 84 | 3 | 20 | 87 | 3 |
| 254B | 5 | 65 | 1 | 72 | 87 | 1 |
| 255B | 190 | 62 | 3 | 5800 | 85 | 4 |
| 256B | 3.4 | 79 | 4 | 53 | 68 | 5 |
| 257B | 8.6 | 78 | 5 | 88 | 82 | 5 |
| 258B | 0.9 | 94 | 3 | 9.7 | 100 | 3 |
| 259B | 0.95 | 95 | 4 | 13 | 91 | 5 |
| 260B | 2.9 | 79 | 4 | 51 | 95 | 3 |
| 261B | 0.96 | 100 | 2 | 10 | 120 | 3 |
| 262B | 1.6 | 83 | 6 | 28 | 88 | 4 |
| 263B | 33 | 80 | 3 | 670 | 110 | 3 |
| 264B | >10000 | 85 | 3 | | | |
| 265B | >15000 | 100 | 3 | | | |
| 266B | >16000 | 100 | 3 | | | |
| 267B | >19000 | 100 | 3 | | | |
| 268B | >20000 | | 1 | | | |
| 269B | >20000 | | 1 | | | |
| 270B | >20000 | | 1 | | | |
| 271B | >20000 | | 1 | | | |
| 272B | >20000 | | 1 | | | |
| 273B | >20000 | | 1 | | | |
| 274B | >20000 | | 1 | | | |
| 275B | >20000 | | 1 | | | |
| 276B | >20000 | | 1 | | | |
| 277B | 8.1 | 64 | 3 | 230 | 77 | 3 |
| 278B | 9.4 | 69 | 3 | 94 | 92 | 3 |
| 279B | 11 | 71 | 4 | 410 | 75 | 3 |
| 280B | 11 | 65 | 2 | 380 | 96 | 3 |
| 281B | 14 | 80 | 3 | 270 | 92 | 1 |
| 282B | 15 | 83 | 3 | 230 | 110 | 3 |
| 283B | 15 | 83 | 4 | 270 | 88 | 3 |
| 284B | 18 | 82 | 4 | 270 | 83 | 3 |
| 285B | 21 | 82 | 6 | 200 | 81 | 3 |
| 286B | 25 | 78 | 3 | 330 | 96 | 3 |
| 287B | 28 | 80 | 3 | 450 | 89 | 3 |
| 288B | 30 | 77 | 3 | 360 | 89 | 3 |
| 289B | 36 | 73 | 3 | 1100 | 79 | 3 |
| 290B | 44 | 72 | 3 | 490 | 98 | 2 |
| 291B | 54 | 77 | 3 | 1200 | 82 | 3 |
| 292B | 55 | 71 | 3 | 1700 | 99 | 3 |
| 293B | 56 | 81 | 8 | 590 | 85 | 4 |
| 294B | 72 | 76 | 3 | 1500 | 81 | 3 |
| 295B | 87 | 83 | 3 | 3100 | 110 | 3 |
| 296B | 96 | 86 | 6 | 1400 | 94 | 4 |
| 297B | 110 | 70 | 5 | 3500 | 95 | 3 |
| 298B | 110 | 74 | 6 | 2700 | 96 | 4 |
| 299B | 110 | 79 | 6 | 2200 | 89 | 4 |
| 300B | 120 | 93 | 3 | 1300 | 96 | 3 |
| 301B | 120 | 80 | 6 | 1500 | 92 | 4 |
| 302B | 130 | 80 | 3 | | | |
| 303B | 170 | 77 | 3 | | | |
| 304B | 190 | 74 | 5 | | | |
| 305B | 190 | 86 | 3 | | | |
| 306B | 190 | 80 | 5 | 3600 | 93 | 3 |
| 307B | 210 | 77 | 3 | | | |
| 308B | 290 | 74 | 3 | | | |
| 309B | 300 | 66 | 3 | | | |
| 310B | 320 | 79 | 3 | | | |
| 311B | 350 | 71 | 3 | | | |
| 312B | 380 | 68 | 3 | | | |
| 313B | 380 | 75 | 3 | | | |
| 314B | 390 | 69 | 2 | | | |
| 315B | 440 | 86 | 3 | | | |
| 316B | 450 | 71 | 3 | 5600 | 76 | 3 |
| 317B | 500 | 69 | 3 | | | |
| 318B | 520 | 80 | 3 | | | |
| 319B | 1100 | 47 | 4 | | | |
| 320B | 1100 | 65 | 1 | | | |
| 321B | 1100 | 81 | 3 | | | |
| 322B | 1400 | 97 | 3 | | | |
| 323B | 1600 | 80 | 3 | | | |
| 324B | 1900 | 79 | 3 | | | |
| 325B | 2200 | 87 | 4 | | | |
| 326B | 2500 | 71 | 3 | | | |
| 327B | 2900 | 88 | 3 | | | |
| 328B | 3100 | 51 | 3 | | | |
| 329B | 4000 | 91 | 3 | | | |
| 330B | 9300 | 100 | 3 | | | |
| 331B | 63 | 78 | 3 | 2000 | 82 | 3 |
| 332B | 5.3 | 76 | 3 | 21 | 84 | 3 |
| 333B | 0.7 | 86 | 4 | 10 | 89 | 6 |
| 334B | 30 | 73 | 4 | 570 | 93 | 8 |
| 335B | 5 | 82 | 4 | 41 | 77 | 4 |
| 336B | 150 | 68 | 3 | 5300 | 80 | 4 |
| 337B | 560 | 72 | 3 | | | |
| 338B | >20000 | | 1 | | | |
| 339B | 36 | 71 | 3 | 1600 | 82 | 3 |
| 340B | 600 | 70 | 3 | | | |
| 341B | 250 | 64 | 4 | | | |
| 342B | 1300 | 42 | 3 | | | |
| 343B | 510 | 64 | 3 | | | |
| 344B | 37 | 69 | 3 | 1200 | 83 | 3 |
| 345B | 6.1 | 89 | 4 | 150 | 97 | 3 |
| 346B | 21 | 79 | 3 | 540 | 80 | 3 |
| 347B | 6.9 | 78 | 4 | 170 | 96 | 3 |
| 348B | 54 | 81 | 4 | 2100 | 100 | 3 |
| 349B | 1200 | 58 | 3 | | | |
| 350B | 420 | 81 | 3 | | | |
| 351B | 5.2 | 66 | 3 | 120 | 80 | 3 |
| 352B | 46 | 66 | 4 | 1400 | 83 | 3 |
| 353B | 5.6 | 80 | 3 | 200 | 89 | 3 |
| 354B | 94 | 78 | 3 | 1500 | 69 | 3 |
| 355B | 6.3 | 83 | 3 | 110 | 87 | 2 |
| 356B | 100 | 78 | 3 | 1100 | 75 | 3 |
| 357B | 28 | 82 | 3 | 350 | 70 | 3 |
| 358B | 4.2 | 69 | 3 | 77 | 75 | 3 |
| 359B | 1.9 | 63 | 3 | 55 | 75 | 3 |
| 360B | 19 | 72 | 4 | 380 | 84 | 3 |
| 361B | 3.3 | 73 | 4 | 54 | 81 | 3 |
| 362B | 12 | 64 | 3 | 270 | 84 | 3 |
| 363B | 7 | 65 | 3 | 140 | 71 | 3 |
| 364B | 1.4 | 77 | 7 | 20 | 79 | 3 |

### Example 3: Effect of Chronic Coadministration of GIPR Antagonist Compound 1 and Liraglutide on Body Weight, Body Composition, and Food Intake in High Fat Diet Fed Mice

Compound 1 is a compound within the scope of Formula I, comprising a 4′-(1-(phenylcarbamoyl)pyrrolidine-2-carboxamido)-[1,1′-biphenyl]-4-carboxylic acid core.

Sixty 6-7 week old male hGIPR KI (C57Bl/6NTac Background) mice (approximately 18-19 weeks old, approximately 43-44 g) were singly housed in a Reverse 12 h light; 12 hr dark cycle (10 pm-10 am) at standard temperature (22° C.) with ad libitum access to 60% high fat diet (Research diets: D12492) and water. Mice were led in on HFD for 12 weeks prior to study start. Body weight and food intake were measured using Sartorius Practum® Precision Balance scales. Mice were acclimated by PO sham dosing with 100 μL of water for three days prior to study start. Baseline body weight was used to randomize mice into four dosing groups. Baseline body composition was also measured via EchoMRI.

From days 0-20, body weights were measured daily and food intake was measured biweekly (Tuesdays and Fridays) using the hopper method. Mice were co-administered with an oral dose QD with vehicle 1 (2% Tween80:98% (0.5% w/v Methylcellulose A4M in DI Water) (v/v)) or 30 mg/kg Compound 1, and a subcutaneous dose QD with vehicle 2 (1.35% Propylene Glycol/98.65% Dulbecco's 0.01 M PBS pH=7.4 (v/v)) or 0.05 mg/kg liraglutide.

On Day 20, to measure exposure of Compound 1 and liraglutide after chronic dosing, approximately 40 μL blood samples were collected 1 and 24 hours post dosing from a subset of mice from each dosing group (n=5). Blood samples were placed into dipotassium ethylenediaminetetraacetic acid (EDTA) tubes and centrifuged at 10,000×g for 10 minutes. Plasma samples were acidified by adding 2 μL of 400 mg/mL citric acid to 19 μL of plasma in 2 mL tubes. The citric acid was prepared by dissolving 2 g of citric acid in 5 mL sterile RODI water. 400 mg/mL citric acid was passed through a Millex® filter unit, aliquoted to 1.7 mL Eppendorf tubes, and stored in 4° C. refrigerator with a shelf life of six months. 20 μL of acidified plasma was transferred into 2 mL tubes and stored in a −80° C. freezer until they were shipped for analysis. On Day 21, body composition was measured via EchoMRI. On day 23, mice were euthanized via CO₂ inhalation.

Pharmacokinetics: Blood was collected from all animals on study into EDTA tubes at each timepoint (1 and 24 HPD) and kept on wet ice until centrifuging at 10,000×g for 10 minutes at 4° C. Resulting plasma was collected and acidified prior to freezing (−80° C.) until analysis. The Cmax, Tmax, and AUC Were calculated for all animals.

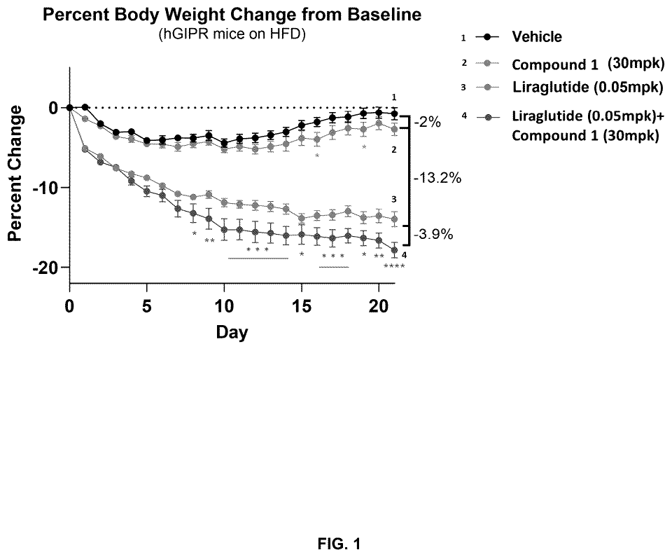

FIG. 1 shows the percent body weight change from baseline for hGIPR mice on a high fat diet treated with Compound 1 and liraglutide. The data show that hGIPR mice fed a combination of liraglutide (0.05 mpk)+Compound 1 (30 mpk) exhibited 17.1% more weight loss than mice administered Compound 1 (30 mpk) alone. The data also show that hGIPR mice fed a combination of liraglutide (0.05 mpk)+Compound 1 (30 mpk) exhibited 3.9% more weight loss than mice administered liraglutide (0.05 mpk) alone. The data demonstrate that combination therapy comprising a GIPR antagonist compound of the disclosure or a pharmaceutically acceptable salt thereof and a GLP-1R agonist small molecule compound or a pharmaceutically acceptable salt thereof may be more effective in treating a disease or condition compared to treatment with a GIPR antagonist compound of the disclosure or a pharmaceutically acceptable salt thereof alone or a GLP-1R agonist small molecule compound or a pharmaceutically acceptable salt thereof alone.

### Example 4: Effect of Chronic Coadministration of a GIPR Antagonist Compound (Compound X) and the GLP-1R Agonist Liraglutide on Body Weight, Body Composition, and Food Intake in High Fat Diet Fed Mice

Compound X is a compound within the scope of Formula I, comprising a 4′-(1-(phenylcarbamoyl) pyrrolidine-2-carboxamido)-[1,1′-biphenyl]-4-carboxylic acid core, which has a different structure than Compound 1. Sixty 8-10 week old male hGIPR KI (C57Bl/6NTac Background) mice (approximately 23-25 weeks old, approximately 46 g) were singly housed in a Reverse 12 h light; 12 hr dark cycle (10 pm-10 am) at standard temperature (22° C.) with ad libitum access to 60% high fat diet (Research diets: D12492) and water. Mice were led in on HFD for 15 weeks prior to study start. Body weight and food intake were measured using Sartorius Practum® Precision Balance scales. Mice were acclimated by PO sham dosing with 100 μL of water for four days prior to study start. Baseline body weight was used to randomize mice into four dosing groups. Baseline body composition was also measured via EchoMRI.

From days 0-28, body weights were measured daily and food intake was measured biweekly (Tuesdays and Fridays) using the hopper method. Mice were co-administered with an oral dose QD with vehicle 1 (2% Tween80: 98% (0.5% w/v Methylcellulose A4M in DI Water) (v/v)) or 30 mg/kg Compound x, and a subcutaneous dose QD with vehicle 2 (1.35% Propylene Glycol/98.65% Dulbecco's 0.01 M PBS pH=7.4 (v/v)) or 0.05 mg/kg liraglutide.

On Day 26, body composition was measured via EchoMRI.

On Day 28, to measure exposure of Compound x and Liraglutide after chronic dosing, approximately 40 μL blood samples were collected 1 and 24 hours post dosing from a subset of mice from each dosing group (n=5). Blood samples were placed into dipotassium ethylenediaminetetraacetic acid (EDTA) tubes and centrifuged at 10,000×g for 10 minutes.

Plasma samples were acidified by adding 1 μL of 400 mg/mL citric acid to 9 μL of plasma in 2 mL tubes. The citric acid was prepared by dissolving 2 g of citric acid in 5 mL sterile RODI water. 400 mg/mL citric acid was passed through a Millex® filter unit, aliquoted to 1.7 mL Eppendorf tubes, and stored in 4° C. refrigerator with a shelf life of six months. 10 μL of acidified plasma was transferred into 2 mL tubes and stored in a −80° C. freezer until they were shipped for analysis. On day 29, mice were euthanized via CO₂ inhalation.

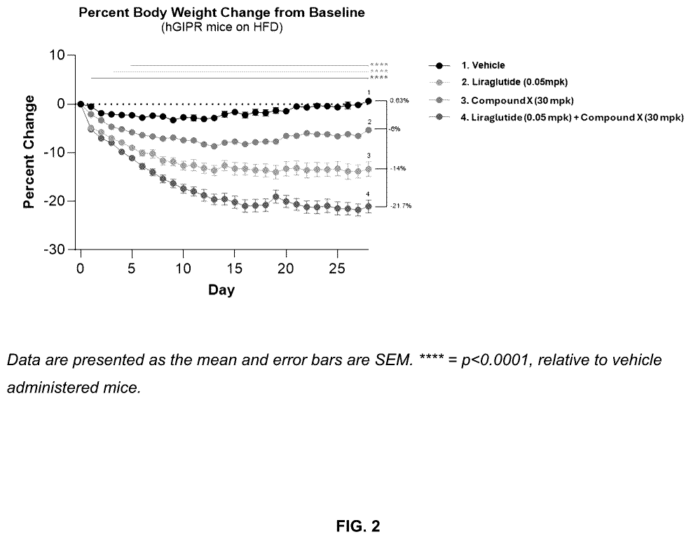

FIG. 2 shows the percent body weight change from baseline for hGIPR mice on a high fat diet treated with Compound X and liraglutide. The data show that hGIPR mice administered a combination of liraglutide (0.05 mpk)+Compound X (30 mpk) exhibited 14.7% more weight loss than mice administered Compound x (30 mpk) alone. The data also show that hGIPR mice administered a combination of Liraglutide (0.05 mpk)+Compound x (30 mpk) exhibited 7.7% more weight loss than mice administered liraglutide (0.05 mpk) alone. The data demonstrate that combination therapy comprising a GIPR antagonist compound of the disclosure or a pharmaceutically acceptable salt thereof and a GLP-1R agonist peptide or a pharmaceutically acceptable salt thereof may be more effective in treating a disease or condition compared to treatment with a GIPR antagonist compound of the disclosure or a pharmaceutically acceptable.

To determine statistical significance, a mixed effects model with appropriate fixed, random, correlation, and variance structure was used for longitudinal outcomes. For multiple group outcomes, ANOVA with appropriate adjustments for violations of homoscedasticity or normality was used. All residuals were evaluated for meeting the normality assumption with the Shapiro-Wilks Test and Q-Q Plots and the sphericity and homoscedasticity assumption by the residual plots and Bartlett's test. FDR adjustment method was used for selected comparisons. All analyses performed with R version 4.1.0.

### Example 5: Effect of GIPR Antagonist Compound 1 and Danuglipron Combination Study on Body Weight in Non-Human Primate

The combination effect of GIPR antagonist Compound 1 and 2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid (danuglipron) on body weight following chronic daily oral administration was evaluated in male and female cynomolgus monkeys. Briefly, 6 male and female cynomolgus monkeys (Envigo Global Services, Inc. Denver, PA) at 3-5 years of age fed a standard diet of pelleted food (Certified Primate Diet 2050C, Envigo Teklad GlobalDiet) supplemented with vegetables and/or fruit were singly housed and acclimated to laboratory environment conditions for a minimum of 30 days prior to the initiation of dosing. Three animals per sex were allocated to receive either vehicle (2% (v/v) low peroxide polysorbate 80 in 0.5% (w/v) methylcellulose in purified water) or the combination of Compound 1 (as lysine salt in 2% (v/v) low peroxide polysorbate 80 in 0.5% (w/v) methylcellulose in purified water daily) and danuglipron (2% (v/v) low peroxide polysorbate 80 in 0.5% (w/v) methylcellulose in purified water). Within the vehicle group on dosing phase days 1-14 animals were administered vehicle by oral gavage BID at a dose volume of 10 mL/kg/dose (AM) and 10 mL/kg/dose (PM). On dosing phase days 15-112 (15-111 females) animals were administered vehicle by oral gavage BID at a dose volume of 10 mL/kg/dose (AM) and 5 mL/kg/dose (PM).

Within the combination treatment arm on dosing phase days 1-7 animals were administered danuglipron (20 [10 BID] mg/kg/day) and Compound 1 (5 [2.5 BID] mg/kg/day) by oral gavage. On dosing phase days 8-14 animals were administered danuglipron (50 [25 BID] mg/kg/day) and Compound 1 (5 [2.5 BID] mg/kg/day) by oral gavage. On dosing phase days 15-112 (15-111 females) animals were administered danuglipron (100 mg/kg/day [QD]—AM only) and Compound 1 (5 [2.5 BID] mg/kg/day) by oral gavage. The order of dose administration was danuglipron immediately followed by Compound 1. Body weight measurements were obtained pre-dose on day 1 and three times weekly thereafter (performed on Monday, Wednesday, and Friday) until day 112 (day 111 females).

Table 7 summarizes the pre and post treatment phase body weight measurements and demonstrates that in animals co-administered danuglipron and Compound 1 there was lower body weight gain in males compared with controls and there was body weight loss in females, whereas control females gained weight. The data show that combination treatment of a GIPR antagonist of the disclosure (e.g., Compound 1), or a pharmaceutically acceptable salt thereof, and a GLP-1 agonist of the disclosure (e.g., danuglipron), or a pharmaceutically acceptable salt thereof, may be effective in reducing body weight and treating conditions disclosed herein.

TABLE 7
The effect of combination treatment of Compound 1 and danuglipron
on body weight in cynomolgus monkeys

| | | Pre-dose | | Percent | Percent |
|---|---|---|---|---|---|
| | | Mean Body | Post-dose* | change | Change |
| | Treatment | Weight | Mean Body | from | from |
| Sex | (n/group) | (kg) | Weight (kg) | Pre-dose | Vehicle |
| Males | Vehicle (n = 5) | 5.66 | 6.62 | +17% | |
| | Combination | 5.60 | 6.00 | +7% | −10% |
| | (n = 3) | | | | |
| Females | Vehicle (n = 5) | 4.06 | 4.40 | +8% | |
| | Combination | 3.97 | 3.80 | −4% | −12% |
| | (n = 3) | | | | |

*study day 112 for males and study day 111 for females

### Example 6: Phase 2 Study for the Coadministration of GIPR Antagonist 4A of the Disclosure Danuglipron

A Phase 2 study is conducted to evaluate the efficacy and safety of a range of doses of the GIPR antagonist 4A and danuglipron over a treatment period of approximately 8 months. GIPR antagonist 4A is administered once daily, and danuglipron is administered once daily as oral tablets. The GIPR antagonist 4A and danuglipron oral tablets are taken around the same time as one another. Alternatively, GIPR antagonist 4A and danuglipron are administered as a single fixed-dose combination oral tablet. The enrolled participants met the required criteria of: 1) BMI 30 kg/m²; or 2) BMI 27 kg/m² with at least one other weight-related condition. The primary outcome measure for efficacy is a change from baseline in body weight at the end of the treatment period. The primary outcome measure for safety is the incidence of treatment-emergent adverse events. Safety is evaluated via measurements, including clinical laboratory tests, electrocardiograms (ECGs), and vital signs.

### Embodiments

The following non-limiting embodiments provide illustrative examples of the invention, but do not limit the scope of the invention.

Embodiment 1. A method of treating a disease or condition comprising administering to a subject in need thereof a therapeutically effective amount of:
a) a glucose-dependent insulinotropic polypeptide receptor (GIPR) antagonist small molecule compound or a pharmaceutically acceptable salt thereof; and
b) a glucagon-like peptide 1 receptor (GLP-1R) agonist small molecule compound or a pharmaceutically acceptable salt thereof,
   wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Embodiment 2. The method of embodiment 1, wherein the administering the GIPR antagonist small molecule compound or a pharmaceutically acceptable salt thereof is oral; and the administering of the GLP-1R agonist small molecule compound is oral.

Embodiment 3. The method of embodiment 1, wherein the administering the GIPR antagonist small molecule compound or a pharmaceutically acceptable salt thereof is oral; and the administering of the GLP-1R agonist small molecule compound is by subcutaneous injection.

Embodiment 4. The method of embodiment 1, wherein the administering the GIPR antagonist small molecule compound or a pharmaceutically acceptable salt thereof is by subcutaneous injection; and the administering of the GLP-1R agonist small molecule compound is oral.

Embodiment 5. The method of embodiment 1, wherein the administering the GIPR antagonist small molecule compound or a pharmaceutically acceptable salt thereof is by subcutaneous injection; and the administering of the GLP-1R agonist small molecule compound is by subcutaneous injection.

Embodiment 6. The method of embodiment 1, wherein the GIPR antagonist small molecule compound has a molecular weight of from about 450 Da to about 550 Da.

Embodiment 7. The method of embodiment 1, wherein the GLP-1R agonist small molecule compound has a molecular weight of from about 450 Da to about 600 Da.

Embodiment 8. A method of treating a disease or condition comprising administering to a subject in need thereof a therapeutically effective amount of:
a) a GIPR antagonist small molecule compound of Formula I:

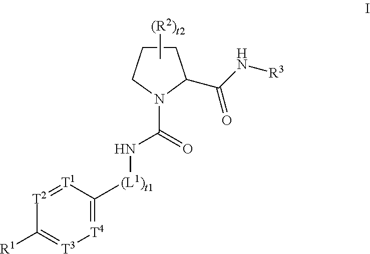

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

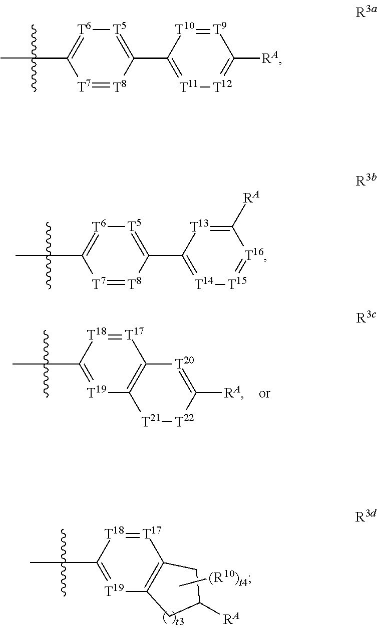

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound or a pharmaceutically acceptable salt thereof,
wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Embodiment 9. The method of embodiment 8, wherein the GIPR antagonist small molecule compound has the Formula Ia:

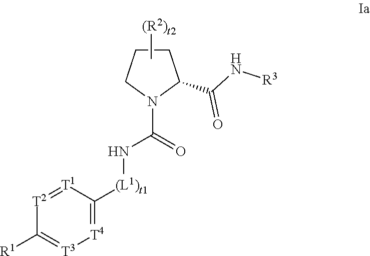

or a pharmaceutically acceptable salt thereof.

Embodiment 10. The method of embodiment 8, wherein the GIPR antagonist small molecule compound has the Formula II:

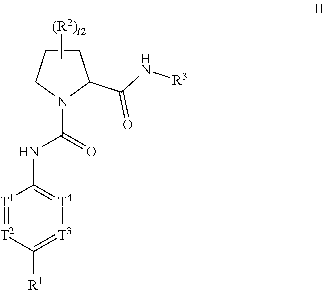

or a pharmaceutically acceptable salt thereof.

Embodiment 11. The method of embodiment 8, wherein the GIPR antagonist small molecule compound has the Formula IIa:

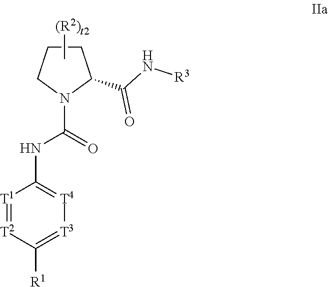

or a pharmaceutically acceptable salt thereof.

Embodiment 12. The method of embodiment 8, wherein the GIPR antagonist small molecule compound has the Formula III or IIIa:

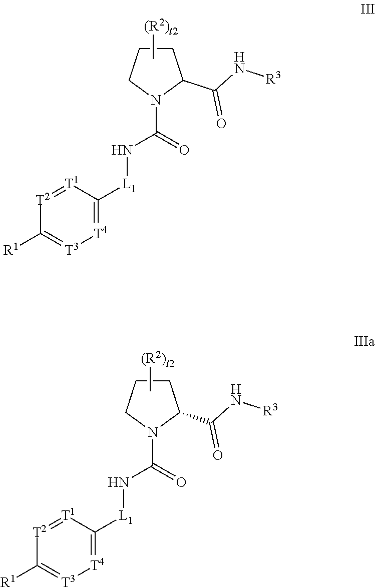

or a pharmaceutically acceptable salt thereof.

Embodiment 13. The method of embodiment 8, wherein the GIPR antagonist small molecule compound has the Formula IV or Formula IVa:

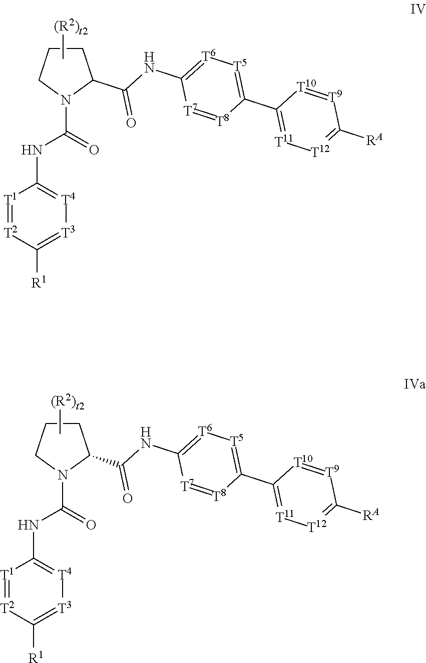

or a pharmaceutically acceptable salt thereof.

Embodiment 14. The method of embodiment 8, wherein the GIPR antagonist small molecule compound has the Formula V or Formula Va:

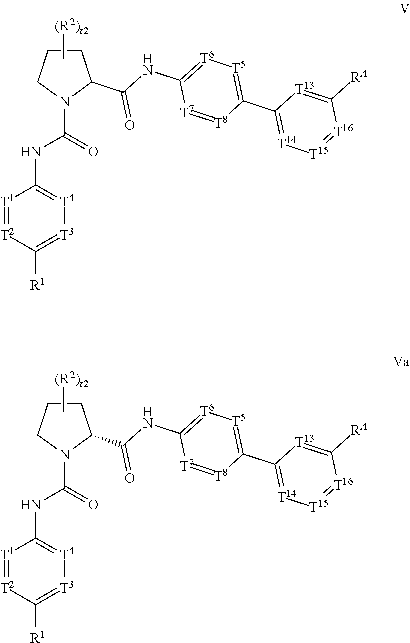

or a pharmaceutically acceptable salt thereof.

Embodiment 15. The method of embodiment 8, wherein the GIPR antagonist small molecule compound has the Formula VI or Formula VIa:

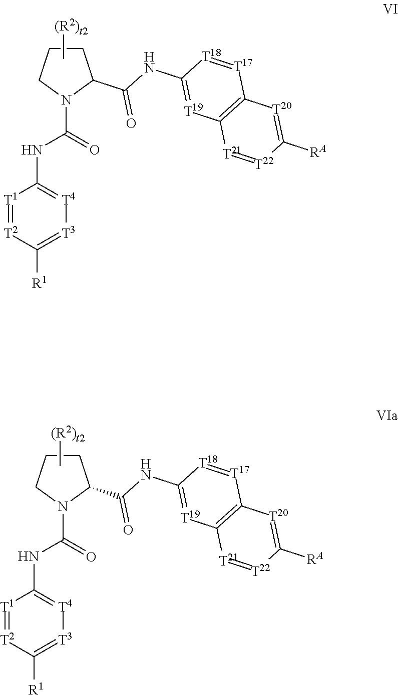

or a pharmaceutically acceptable salt thereof.

Embodiment 16. The method of embodiment 8, wherein the GIPR antagonist small molecule compound has the Formula VII or Formula VIIa:

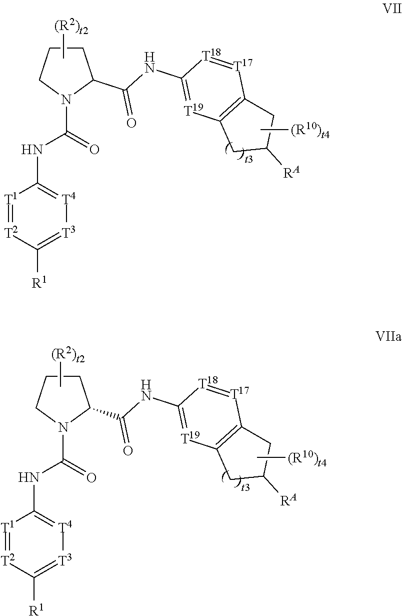

or a pharmaceutically acceptable salt thereof.

Embodiment 17. The method of any one of embodiments 8 to 16, wherein R¹ is cyclopropyl, cyclobutyl, R^{1a}, R^{1b}, or R^{1c},

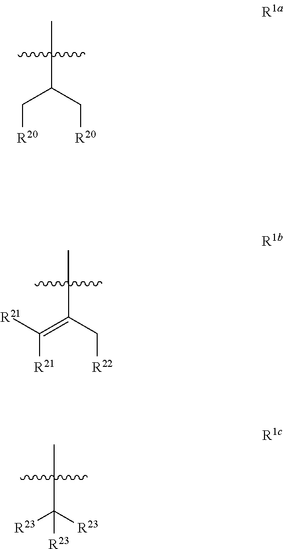

wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy.

Embodiment 18. The method of any one of embodiments 8 to 17, wherein R¹ is propan-2-yl, prop-1-en-2-yl, or cyclopropyl.

Embodiment 19. The method of any one of embodiments 8 to 18, wherein R¹ is propan-2-yl.

Embodiment 20. The method of any one of embodiments 8 to 19, wherein each of T¹, T², T³, and T⁴ is independently CR⁴.

Embodiment 21. The method of any one of embodiments 8 to 19, wherein one of T¹, T², T³, and T⁴ is N, and the other three are each independently CR⁴.

Embodiment 22. The method of any one of embodiments 8 to 21, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-; and t2 is 0, 1, or 2.

Embodiment 23. The method of any one of embodiments 8-14 and 17-22, wherein each of T⁵, T⁶, T⁷, and T⁸ is independently CR⁵.

Embodiment 24. The method of any one of embodiments 8-14 and 17-22, wherein one of T⁵, T⁶, T⁷, and T⁸ is N and the other three are each independently CR⁵.

Embodiment 25. The method of any one of embodiments 8-13 and 17-24, wherein each of T⁹, T¹⁰, T¹¹, and T¹² is independently CR⁶.

Embodiment 26. The method of any one of embodiments 8-13 and 17-24, wherein one of T⁹, T¹⁰, T¹¹, and T¹² is N and the other three are each independently CR⁶.

Embodiment 27. The method of any one of embodiments 8-12, 14, and 17-24, wherein each of T¹³, T¹⁴, T¹⁵, and T¹⁶ is independently CR⁷.

Embodiment 28. The method of any one of embodiments 8-12, 14, and 17-22, wherein one of T¹³, T¹⁴, T¹⁵, and T¹⁶ is N and the other three are each independently CR⁷.

Embodiment 29. The method of any one of embodiments 8-12 and 15-22, wherein each of T¹⁷, T¹⁸, and T¹⁹ is independently CR³.

Embodiment 30. The method of any one of embodiments 8-12 and 15-22, wherein one of T¹⁷, T¹⁸, and T¹⁹ is N, and the other two are each independently CR³.

Embodiment 31. The method of any one of embodiments 8-12, 15, 17-22, 29, and 30, wherein each of T²⁰, T²¹, and T²² is independently CR⁹.

Embodiment 32. The method of any one of embodiments 8-13, 15, 17-22, 29, and 30, wherein one of T²⁰, T²¹, and T²² is N, and the other two are each independently CR⁹.

Embodiment 33. The method of any one of embodiments 8-13, 16, 17-22, 29, and 30, wherein t3 is 2.

Embodiment 34. The method of any one of embodiments 8-13, 15, 17-22, 29, 30, and 33, wherein t4 is 0, 1, or 2; and each R¹⁰ is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

Embodiment 35. The method of any one of embodiments 8-34, wherein R^{A} is —C(═O)—OH.

Embodiment 36. The method of embodiment 8, wherein the GIPR antagonist small molecule compound is selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid; and
3′,5′-difluoro-4′-[(1-{[4-(propan-2-yl)phenyl]carbamoyl}-DL-prolyl)amino][1,1′-biphenyl]-4-carboxylic acid,
   or a pharmaceutically acceptable salt thereof.

Embodiment 37. The method of embodiment 8, wherein the GIPR antagonist small molecule compound is selected from the group consisting of:
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid; and
3′,5′-difluoro-4′-[(1-{[4-(propan-2-yl)phenyl]carbamoyl}-D-prolyl)amino][1,1′-biphenyl]-4-carboxylic acid,
   or a pharmaceutically acceptable salt thereof.

Embodiment 38. The method of embodiment 8, wherein the GIPR antagonist small molecule compound selected from:
4′-[(1-{[4-(propan-2-yl)phenyl]carbamoyl}-DL-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid; and
6-methyl-5-{4-[(1-{[3-methyl-4-(trifluoromethyl)phenyl]carbamoyl}-DL-prolyl)amino]phenyl}pyridine-2-carboxylic acid,
   or a pharmaceutically acceptable salt thereof.

Embodiment 39. The method of embodiment 8, wherein the GIPR antagonist small molecule compound is selected from the group consisting of:
4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid; and
6-methyl-5-{4-[(1-{[3-methyl-4-(trifluoromethyl)phenyl]carbamoyl}-D-prolyl)amino]phenyl}pyridine-2-carboxylic acid,

or a pharmaceutically acceptable salt thereof.

Embodiment 40. The method of embodiment 8, wherein the GIPR antagonist small molecule compound is selected from the group consisting of:
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid; and
3-[6-({1-[(4-cyclobutylphenyl)carbamoyl]-DL-prolyl}amino)pyridin-3-yl]benzoic acid,

or a pharmaceutically acceptable salt thereof.

Embodiment 41. The method of embodiment 8, wherein the GIPR antagonist small molecule compound is selected from the group consisting of:
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof.

Embodiment 42. The method of any one of the preceding embodiments, wherein the condition is obesity.

Embodiment 43. The method of any one of the preceding embodiments, wherein the condition is diabetes.

Embodiment 44. The method of any of the preceding embodiments, wherein the therapeutically effective amount of the GIPR antagonist small molecule compound is from about 1 mg to about 100 mg.

Embodiment 45. The method of any of the preceding embodiments, wherein the therapeutically effective amount of the GIPR antagonist small molecule compound is from about 1 mg to about 25 mg.

Embodiment 46. The method of any of the preceding embodiments, wherein the GIPR antagonist small molecule compound or a pharmaceutically acceptable salt thereof is administered orally.

Embodiment 47. The method of any of the preceding embodiments, wherein the GIPR antagonist small molecule compound or a pharmaceutically acceptable salt thereof is administered by subcutaneous injection.

Embodiment 48. The method of any one of the preceding embodiments, wherein the GIPR antagonist small molecule compound or a pharmaceutically acceptable salt thereof is administered daily.

Embodiment 49. The method of any one of the preceding embodiments, wherein the GIPR antagonist small molecule compound or a pharmaceutically acceptable salt thereof is administered weekly.

Embodiment 50. The method of any one of embodiments 5-47, wherein the GLP-1R agonist small molecule compound is a compound of Formula B-I:

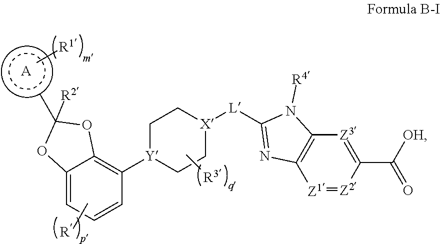

or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O′}, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}′, and —N(R^{N}′)₂;
R^{5′} is a 4- to 6-membered heterocycloalkyl, wherein said heterocycloalkyl may be substituted with 0 to 2 substituents as valency allows independently selected from:
   0 to 1 oxo (═O),
   0 to 1 —CN,
   0 to 2 F atoms, and
   0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O}′;
R^{6′} is a 5- to 6-membered heteroaryl, wherein said heteroaryl may be substituted with 0 to 2 substituents as valency allows independently selected from:
   0 to 2 halogens,
   0 to 1 substituent selected from —OR^{O′} and —N(R^{N}′)₂, and
   0 to 2 —C₁₋₃alkyl, wherein the alkyl may be substituted with 0 to 3 substituents as valency allows independently selected from:
   0 to 3 F atoms, and
   0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z′}, or one of Z^{1′}, Z^{2′}, and Z^{3′} is N and the other two are —CR^{Z}′; and
each R^{Z}′ is independently H, F, Cl, or —CH₃.

Embodiment 51. The method of any one of embodiments 5-47, wherein the GLP-1R agonist small molecule compound is a compound of Formula B-II:

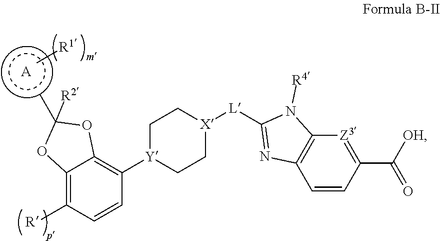

or a pharmaceutically acceptable salt thereof, wherein
R′ is F;
p′ is 0 or 1;
ring A is phenyl or pyridinyl;
m′ is 0, 1, or 2;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or CH₃;
X′-L′ is N—CH₂, or cyclopropyl;
Y′ is CH or N;
Z^{3′} is —CR^{Z′} or N; and
R^{Z}′ is H, F, Cl, or —CH₃.

Embodiment 52. The method of any one of embodiments 5-47, wherein the GLP-1R agonist small molecule compound is a compound of Formula B-III:

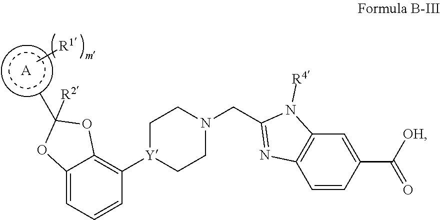

or a pharmaceutically acceptable salt thereof, wherein
ring A is phenyl or pyridinyl;
m′ is 0, 1, or 2;
each R^{1′} is independently selected from F, Cl, and —CN;
R^{2′} is H or CH₃; and
Y′ is CH or N.

Embodiment 53. The method of any one of embodiments 50-52, wherein R₄ is —CH₂—R⁵, wherein R⁵ is the 4- to 5-membered heterocycloalkyl, wherein said heterocycloalkyl may be substituted with 0 to 2 substituents as valency allows independently selected from:
0 to 2 F atoms, and
0 to 1 substituent selected from —OCH₃ and —CH₂OCH₃;
or a pharmaceutically acceptable salt thereof.

Embodiment 54. The method of any one of embodiments 50-52, wherein R₄ is —CH₂—R⁶, wherein R⁶ is the 5-membered heteroaryl, wherein said heteroaryl may be substituted with 0 to 2 substitutents as valency allows independently selected from:
0 to 2 halogens, wherein the halogen is independently selected from F and C,
0 to 1 —OCH₃, and
0 to 1 —CH₃, —CH₂CH₃, —CF₃, or —CH₂CH₂OCH₃;

or a pharmaceutically acceptable salt thereof.

Embodiment 55. The method of any one of embodiments 50-52, wherein R² is H, or a pharmaceutically acceptable salt thereof.

Embodiment 56. The method of embodiment 50, wherein the GLP-1R agonist small molecule compound is selected from the group consisting of:
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[(2R)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid,
   or a pharmaceutically acceptable salt thereof.

Embodiment 57. The method of any one of embodiments 5-47, wherein the GLP-1R agonist small molecule compound is a compound of Formula C-I:

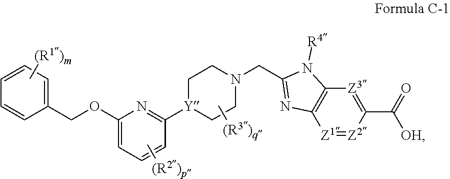

or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{5″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
   0 to 3 F atoms, and
   0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
   each R^{Z″} is independently H, F, Cl, or —CH₃.

Embodiment 58. The method of any one of embodiments 5-47, wherein the GLP-1R agonist small molecule compound is a compound of Formula C-II:

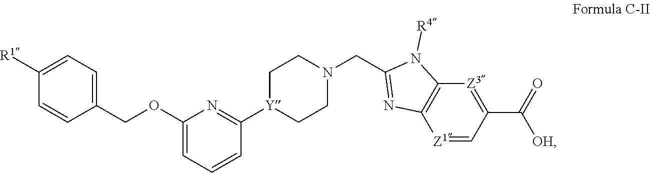

or a pharmaceutically acceptable salt thereof, wherein
m″ is 0 or 1;
R^{2″} is F;
p″ is 0, or 1; and
q″ is 0 or 1.

In some embodiments, disclosed herein is a compound of Formula C-I or Formula C-II, wherein:
m″ is 0 or 1;
q″ is 0 or 1; and
R^{3″} is —F, —CH₃, —CH₂CH₃, —CH₂OH, —CF₃, isopropyl, or cyclopropyl,
   or a pharmaceutically acceptable salt thereof.

Embodiment 59. The method of any one of embodiments 5-47, wherein the GLP-1R agonist small molecule compound is a compound of Formula C-III:

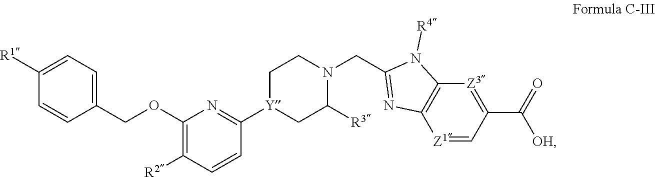

or a pharmaceutically acceptable salt thereof, wherein
m″ is 0 or 1;
R^{2″} is F;
p″ is 0, or 1;
R^{3″} is —C₁₋₂alkyl, wherein —C₁₋₂alkyl may be substituted as valency allows with 0 to 3 F atoms; and
q″ is 0 or 1.

Embodiment 60. The method of any one of embodiments 57-59, wherein each R¹ is independently F, Cl, —CN, —CH₃, or —CF₃, or a pharmaceutically acceptable salt thereof.

Embodiment 61. The method of any one of embodiments 57-60, wherein the heterocycloalkyl is

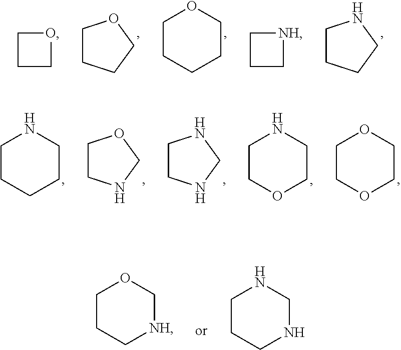

wherein the heterocycloalkyl may be substituted with 0 to 2 substituents as valency allows independently selected from:
0 to 1 oxo (O═),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be independently substituted with 0 to 3 substituents as valency allows independently selected from:
   0 to 3 F atoms,
   0 to 1 —CN, and
   0 to 1 —OR^{O},
      or a pharmaceutically acceptable salt thereof.

Embodiment 62. The method of any one of embodiments 57-60, wherein R₄ is —CH₂—R⁵, wherein R⁵ is the 4- to 5-membered heterocycloalkyl, wherein said heterocycloalkyl may be substituted with 0 to 2 substituents as valency allows independently selected from: 0 to 2 F atoms, and 0 to 1 substituent selected from —OCH₃ and —CH₂OCH₃, or a pharmaceutically acceptable salt thereof.

Embodiment 63. The method of any one of embodiments 57-60, wherein heteroaryl is

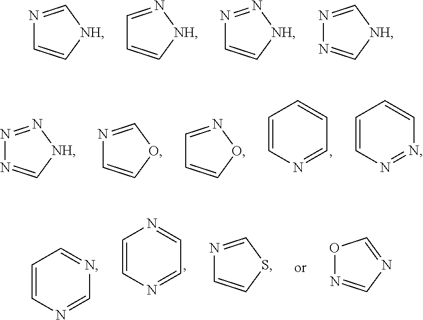

and wherein said heteroaryl may be substituted with 0 to 2 substituents as valency allows independently selected from:
0 to 3 halogens,
0 to 1 substituent selected from —OR^{O} and —N(R^{N})₂, and
0 to 2 —C₁₋₃alkyl, wherein the alkyl may be substituted with 0 to 3 substituents as valency allows independently selected from:
   0 to 3 F atoms, and
   0 to 1 —OR^{O};
      or a pharmaceutically acceptable salt thereof.

Embodiment 64. The method of any one of embodiments 57-60 and 63, wherein R₄ is —CH₂—R⁶, wherein R⁶ is the 5-membered heteroaryl, wherein said heteroaryl may be substituted with
0 to 2 substitutents as valency allows independently selected from:
0 to 2 halogens, wherein the halogen is independently selected from F and Cl,
0 to 1 —OCH₃, and
0 to 1 —CH₃, —CH₂CH₃, —CF₃, or —CH₂CH₂OCH₃;
   or a pharmaceutically acceptable salt thereof.

Embodiment 65. The method of embodiment 57, wherein the GLP-1R agonist small molecule compound is selected from the group consisting of:
2-[(4-{6-[(4-chloro-2-fluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2R)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid;
2-{[4-(6-{[(4-cyano-2-fluorophenyl)(methyl-d2)]oxy}pyridin-2-yl)piperidin-1-yl]methyl}-1-[(2S)-oxetan-2-ylmethyl]-1H-benzimidazole-6-carboxylic acid;
2-[(4-{6-[(4-cyano-2-fluorobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-chloro-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(2,4-difluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(2,4-difluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-chloro-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-chloro-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3S)-tetrahydrofuran-3-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2R)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2R)-tetrahydrofuran-2-ylmethyl]-1H-benzimidazole-6-carboxylic acid;
2-{[(2S)-4-{6-[(2,4-difluorobenzyl)oxy]-5-fluoropyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(2,4-difluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyanobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-5-ylmethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-5-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-cyano-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-4-ylmethyl)-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,2-oxazol-5-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,2-oxazol-3-ylmethyl)-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyano-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyanobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-cyanobenzyl)oxy]pyridin-2-yl}piperazin-1-yl)methyl]-1-(2-methoxyethyl)-1H-benzimidazole-6-carboxylic acid;
2-{[(2S)-4-{6-[(4-cyanobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-chloro-2-fluorobenzyl)oxy]-5-fluoropyridin-2-yl}-2-methylpiperazin-1-yl]methyl}-1-(2-methoxyethyl)-1H-benzimidazole-6-carboxylic acid;
2-{[(2S)-4-{6-[(4-cyanobenzyl)oxy]-5-fluoropyridin-2-yl}-2-methylpiperazin-1-yl]methyl}-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-1-(2-methoxyethyl)-1H-benzimidazole-6-carboxylic acid; and
2-[(4-{6-[(4-chloro-2-fluorobenzyl)oxy]pyridin-2-yl}piperidin-1-yl)methyl]-1-[(1-methoxycyclobutyl)methyl]-1H-benzimidazole-6-carboxylic acid;
   or a pharmaceutically acceptable salt thereof.

Embodiment 66. A method of treating a disease or condition comprising administering to a subject in need thereof a therapeutically effective amount of:
a) a GIPR antagonist small molecule compound or a pharmaceutically acceptable salt thereof selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid, or a pharmaceutically acceptable salt thereof; and
   b) a GLP-1R agonist small molecule compound selected from the group consisting of:
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[(2R)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid,
   or a pharmaceutically acceptable salt thereof.

Embodiment 67. A method of treating a disease or condition comprising administering to a subject in need thereof a therapeutically effective amount of:
a) a GIPR antagonist small molecule compound or a pharmaceutically acceptable salt thereof selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof; and
b) a GLP-1R agonist small molecule compound selected from the group consisting of:
2-[(4-{6-[(4-chloro-2-fluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2R)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid;
2-{[4-(6-{[(4-cyano-2-fluorophenyl)(methyl-d2)]oxy}pyridin-2-yl)piperidin-1-yl]methyl}-1-[(2S)-oxetan-2-ylmethyl]-1H-benzimidazole-6-carboxylic acid;
2-[(4-{6-[(4-cyano-2-fluorobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-chloro-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(2,4-difluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(2,4-difluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-chloro-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-chloro-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3S)-tetrahydrofuran-3-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2R)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2R)-tetrahydrofuran-2-ylmethyl]-1H-benzimidazole-6-carboxylic acid;
2-{[(2S)-4-{6-[(2,4-difluorobenzyl)oxy]-5-fluoropyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(2,4-difluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyanobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-5-ylmethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-5-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-cyano-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-4-ylmethyl)-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,2-oxazol-5-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,2-oxazol-3-ylmethyl)-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyano-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyanobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-cyanobenzyl)oxy]pyridin-2-yl}piperazin-1-yl)methyl]-1-(2-methoxyethyl)-1H-benzimidazole-6-carboxylic acid;
2-{[(2S)-4-{6-[(4-cyanobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-chloro-2-fluorobenzyl)oxy]-5-fluoropyridin-2-yl}-2-methylpiperazin-1-yl]methyl}-1-(2-methoxyethyl)-1H-benzimidazole-6-carboxylic acid;
2-{[(2S)-4-{6-[(4-cyanobenzyl)oxy]-5-fluoropyridin-2-yl}-2-methylpiperazin-1-yl]methyl}-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-1-(2-methoxyethyl)-1H-benzimidazole-6-carboxylic acid; and
2-[(4-{6-[(4-chloro-2-fluorobenzyl)oxy]pyridin-2-yl}piperidin-1-yl)methyl]-1-[(1-methoxycyclobutyl)methyl]-1H-benzimidazole-6-carboxylic acid;
   or a pharmaceutically acceptable salt thereof.

Embodiment 68. The method of any one of embodiments 8-67, wherein the GLP-1R agonist small molecule compound or pharmaceutically acceptable salt thereof is administered orally.

Embodiment 69. The method of any one of embodiments 8-67, wherein the GLP-1R agonist small molecule compound or pharmaceutically acceptable salt thereof is administered by subcutaneous injection.

Embodiment 70. The method of any one of embodiments 8-69, wherein the GLP-1R agonist small molecule compound or pharmaceutically acceptable salt thereof is administered once a day.

Embodiment 71. The method of any one of embodiments 8-69, wherein the GLP-1R agonist small molecule compound or pharmaceutically acceptable salt thereof is administered once a week.

Embodiment 72. The method of any one of embodiments 8-69, wherein the GLP-1R agonist small molecule compound or pharmaceutically acceptable salt thereof is administered once every two weeks.

Embodiment 73. The method of any one of embodiments 8-72, wherein the GIPR antagonist small molecule compound or pharmaceutically acceptable salt thereof is administered orally.

Embodiment 74. The method of any one of embodiments 8-72, wherein the GIPR antagonist small molecule compound or pharmaceutically acceptable salt thereof is administered by subcutaneous injection.

Embodiment 75. The method of any one of embodiments 8-74, wherein the GIPR antagonist small molecule compound or pharmaceutically acceptable salt thereof is administered once a day.

Embodiment 76. The method of any one of embodiments 8-74, wherein the GIPR antagonist small molecule compound or pharmaceutically acceptable salt thereof is administered once a week.

Embodiment 77. The method of any one of embodiments 8-74, wherein the GIPR antagonist small molecule compound or pharmaceutically acceptable salt thereof is administered once every two weeks.

Embodiment 78. The method of any of the preceding embodiments, wherein the subject is human.

Embodiment 79. A pharmaceutical composition comprising:
a) a GIPR antagonist small molecule compound of Formula I:

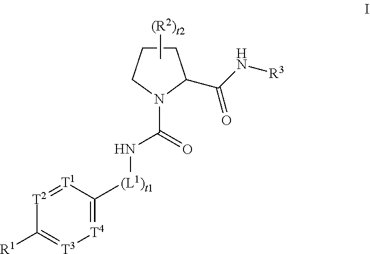

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

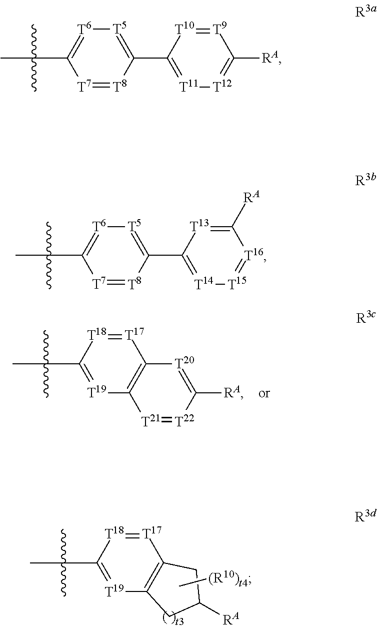

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹ and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula B-I:

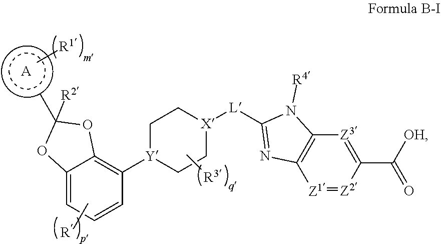

or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O}, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}, and —N(R^{N}′)₂
R^{5′} is a 4- to 6-membered heterocycloalkyl, wherein said heterocycloalkyl may be substituted with 0 to 2 substituents as valency allows independently selected from:
   0 to 1 oxo (═O),
   0 to 1 —CN,
   0 to 2 F atoms, and
   0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
   0 to 3 F atoms,
   0 to 1 —CN, and
   0 to 1 —OR^{O}′;
R^{6′} is a 5- to 6-membered heteroaryl, wherein said heteroaryl may be substituted with 0 to 2 substituents as valency allows independently selected from:
   0 to 2 halogens,
   0 to 1 substituent selected from —OR^{O}′ and —N(R^{N}′)₂, and
   0 to 2 —C₁₋₃alkyl, wherein the alkyl may be substituted with 0 to 3 substituents as valency allows independently selected from:
   0 to 3 F atoms, and
   0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z}′, or one of Z¹′, Z²′, and Z³′ is N and the other two are —CR^{Z}′; and
each R^{Z}′ is independently H, F, Cl, or —CH₃.

Embodiment 80. A pharmaceutical composition comprising:
a) a GIPR antagonist small molecule compound of Formula I:

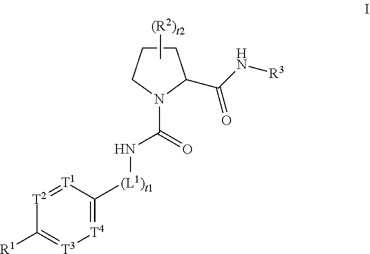

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

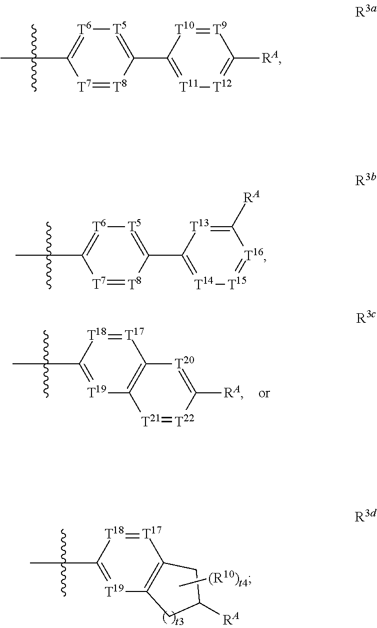

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula C-I:

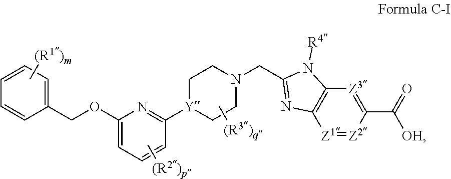

or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{4″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
0 to 3 F atoms,
0 to 1 —CN, and
0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
   0 to 3 F atoms, and
   0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
each R^{Z″} is independently H, F, Cl, or —CH₃.

Embodiment 81. The pharmaceutical composition of embodiments 79 or 80, wherein the GIPR antagonist small molecule is selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof.

Embodiment 82. The pharmaceutical composition of embodiments 79, wherein the GLP-1R agonist small molecule is selected from the group consisting of:
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[(2R)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid, or a pharmaceutically acceptable salt thereof.
Embodiment 83. The pharmaceutical composition of embodiment 80, wherein the GLP-1R agonist small molecule is selected from the group consisting of:
2-[(4-{6-[(4-chloro-2-fluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2R)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(2S)-oxetan-2-ylmethyl]-1H-benzimidazole-6-carboxylic acid;
2-{[4-(6-{[(4-cyano-2-fluorophenyl)(methyl-d2)]oxy}pyridin-2-yl)piperidin-1-yl]methyl}-1-[(2S)-oxetan-2-ylmethyl]-1H-benzimidazole-6-carboxylic acid;
2-[(4-{6-[(4-cyano-2-fluorobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-chloro-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(2,4-difluorobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(2,4-difluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-chloro-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-chloro-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-chloro-2-fluorobenzyl)oxy]pyridin-2-yl}piperidin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperazin-1-yl)methyl]-3-[(2S)-oxetan-2-ylmethyl]-3H-imidazo[4,5-b]pyridine-5-carboxylic acid;
2-[(4-{6-[(4-cyano-2-fluorobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3R)-tetrahydrofuran-3-ylmethyl]-1H-benzimidazole-6-carboxylic acid;
2-[(4-{6-[(4-cyano-2-fluorobenzyl)oxy]pyridin-2-yl}piperidin-1-yl)methyl]-1-[(3S)-tetrahydrofuran-3-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]-5-fluoropyridin-2-yl}piperidin-1-yl)methyl]-1-[(2R)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-oxetan-2-ylmethyl]-1H-benzimidazole-6-carboxylic acid;
2-[(4-{6-[(4-cyanobenzyl)oxy]pyridin-2-yl}piperidin-1-yl)methyl]-1-[(2S)-tetrahydrofuran-2-ylmethyl]-1H-benzimidazole-6-carboxylic acid;
2-[(4-{6-[(4-cyanobenzyl)oxy]pyridin-2-yl}piperidin-1-yl)methyl]-1-[(2R)-tetrahydrofuran-2-ylmethyl]-1H-benzimidazole-6-carboxylic acid;
2-{[(2S)-4-{6-[(2,4-difluorobenzyl)oxy]-5-fluoropyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(2,4-difluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(2S)-oxetan-2-ylmethyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyano-2-fluorobenzyl)oxy]pyridin-2-yl}-2-methylpiperazin-1-yl]methyl}-3-[(2S)-oxetan-2-ylmethyl]-3H-imidazo[4,5-b]pyridine-5-carboxylic acid;
2-{[(2S)-4-{6-[(4-cyanobenzyl)oxy]pyridin-2-yl}-2-methylpiperazin-1-yl]methyl}-1-[(2S)-oxetan-2-ylmethyl]-1H-benzimidazole-6-carboxylic acid;
2-[(4-{6-[(4-cyano-2-fluorobenzyl)oxy]pyridin-2-yl}piperazin-1-yl)methyl]-1-[(2S)-oxetan-2-ylmethyl]-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-methyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-5-ylmethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-5-ylmethyl)-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-2-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-imidazol-5-yl)methyl]-1H-benzimidazole-6-carboxylic acid;
2-[(4-{6-[(4-chloro-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-cyano-2-fluorobenzyl)oxy]pyridin-2-yl}piperazin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-4-ylmethyl)-1H-benzimidazole-6-carboxylic acid;
2-[(4-{6-[(4-chloro-2-fluorobenzyl)oxy]pyridin-2-yl}piperidin-1-yl)methyl]-1-(1,3-oxazol-2-ylmethyl)-1H-benzimidazole-6-carboxylic acid;
2-[(4-{6-[(2,4-difluorobenzyl)oxy]pyridin-2-yl}piperidin-1-yl)methyl]-1-(1,3-oxazol-5-ylmethyl)-1H-benzimidazole-6-carboxylic acid;
2-[(4-{6-[(4-chloro-2-fluorobenzyl)oxy]pyridin-2-yl}piperazin-1-yl)methyl]-1-(1,3-oxazol-5-ylmethyl)-1H-benzimidazole-6-carboxylic acid;
2-[(4-{6-[(4-cyano-2-fluorobenzyl)oxy]pyridin-2-yl}piperazin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-(1,2-oxazol-5-ylmethyl)-1H-benzimidazole-6-carboxylic acid;
2-[(4-{6-[(4-cyano-2-fluorobenzyl)oxy]pyridin-2-yl}piperidin-1-yl)methyl]-1-(1,2-oxazol-3-ylmethyl)-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-{[(2S)-4-{6-[(4-chloro-2-fluorobenzyl)oxy]pyridin-2-yl}-2-methylpiperazin-1-yl]methyl}-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-ethyl-1H-1,2,3-triazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-1-[(1-ethyl-1H-imidazol-5-yl)methyl]-1H-benzimidazole-6-carboxylic acid;
2-[(4-{6-[(4-cyano-2-fluorobenzyl)oxy]pyridin-2-yl}piperidin-1-yl)methyl]-1-[(1-methyl-1H-imidazol-5-yl)methyl]-1H-benzimidazole-6-carboxylic acid;
2-[(4-{6-[(4-chloro-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyano-2-fluorobenzyl)oxy]pyridin-2-yl}piperidin-1-yl)methyl]-3-(1,3-oxazol-2-ylmethyl)-3H-imidazo[4,5-b]pyridine-5-carboxylic acid;
2-[(4-{6-[(4-cyanobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-7-fluoro-1-(2-methoxyethyl)-1H-benzimidazole-6-carboxylic acid;
2-[(4-{6-[(4-cyanobenzyl)oxy]pyridin-2-yl}piperazin-1-yl)methyl]-1-(2-methoxyethyl)-1H-benzimidazole-6-carboxylic acid;
2-{[(2S)-4-{6-[(4-cyanobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-cyano-2-fluorobenzyl)oxy]pyridin-2-yl}-2-methylpiperazin-1-yl]methyl}-1-(2-methoxyethyl)-1H-benzimidazole-6-carboxylic acid;
2-{[(2S)-4-{6-[(4-chloro-2-fluorobenzyl)oxy]-5-fluoropyridin-2-yl}-2-methylpiperazin-1-yl]methyl}-1-(2-methoxyethyl)-1H-benzimidazole-6-carboxylic acid;
2-{[(2S)-4-{6-[(4-cyanobenzyl)oxy]-5-fluoropyridin-2-yl}-2-methylpiperazin-1-yl]methyl}-1-(2-methoxyethyl)-1H-benzimidazole-6-carboxylic acid;
2-[(4-{6-[(4-chloro-2-fluorobenzyl)oxy]pyridin-2-yl}piperidin-1-yl)methyl]-1-(2-methoxyethyl)-1H-benzimidazole-6-carboxylic acid; and
2-[(4-{6-[(4-chloro-2-fluorobenzyl)oxy]pyridin-2-yl}piperidin-1-yl)methyl]-1-[(1-methoxycyclobutyl)methyl]-1H-benzimidazole-6-carboxylic acid;
   or a pharmaceutically acceptable salt thereof.

Embodiment 84. The pharmaceutical composition of any one of embodiments 79-83, wherein the pharmaceutical composition is formulated for oral administration.

Embodiment 85. The pharmaceutical composition of any one of embodiments 79-83, wherein the pharmaceutical composition is formulated for administration by subcutaneous injection.

Embodiment 101. A method of treating a disease or condition comprising administering to a subject in need thereof a therapeutically effective amount of:
a) a glucose-dependent insulinotropic polypeptide receptor (GIPR) antagonist small molecule compound or a pharmaceutically acceptable salt thereof; and
b) a glucagon-like peptide 1 receptor (GLP-1R) agonist small molecule compound or a pharmaceutically acceptable salt thereof,
   wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Embodiment 102. The method of embodiment 101, wherein the administering the GIPR antagonist small molecule compound or a pharmaceutically acceptable salt thereof is oral; and the administering of the GLP-1R agonist small molecule compound is oral.

Embodiment 103. The method of embodiment 101, wherein the administering the GIPR antagonist small molecule compound or a pharmaceutically acceptable salt thereof is oral; and the administering of the GLP-1R agonist small molecule compound is by subcutaneous injection.

Embodiment 104. The method of embodiment 101, wherein the administering the GIPR antagonist small molecule compound or a pharmaceutically acceptable salt thereof is by subcutaneous injection; and the administering of the GLP-1R agonist small molecule compound is oral.

Embodiment 105. The method of embodiment 101, wherein the administering the GIPR antagonist small molecule compound or a pharmaceutically acceptable salt thereof is by subcutaneous injection; and the administering of the GLP-1R agonist small molecule compound is by subcutaneous injection.

Embodiment 106. The method of embodiment 101, wherein the GIPR antagonist small molecule compound has a molecular weight of from about 450 Da to about 550 Da.

Embodiment 107. The method of embodiment 101, wherein the GLP-1R agonist small molecule compound has a molecular weight of from about 450 Da to about 1000 Da.

Embodiment 108. A method of treating a disease or condition comprising administering to a subject in need thereof a therapeutically effective amount of:
a) a GIPR antagonist small molecule compound of Formula I:

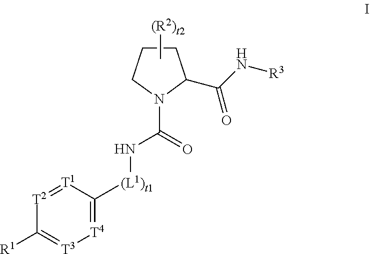

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

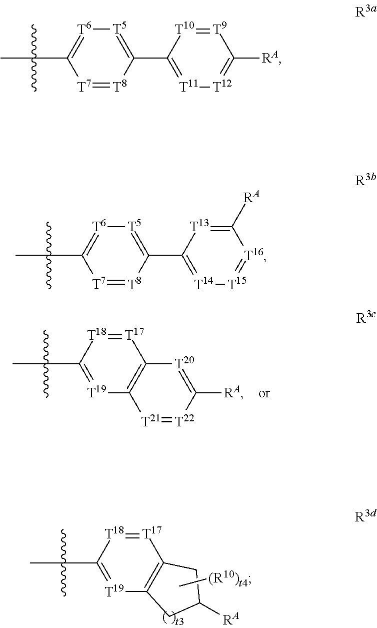

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound or a pharmaceutically acceptable salt thereof,
wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

Embodiment 109. The method of embodiment 108, wherein the GIPR antagonist small molecule compound has the Formula Ia:

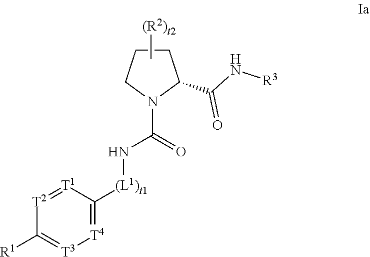

or a pharmaceutically acceptable salt thereof.

Embodiment 110. The method of embodiment 108, wherein the GIPR antagonist small molecule compound has the Formula II:

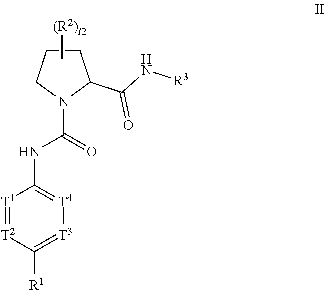

or a pharmaceutically acceptable salt thereof.

Embodiment 111. The method of embodiment 108, wherein the GIPR antagonist small molecule compound has the Formula IIa:

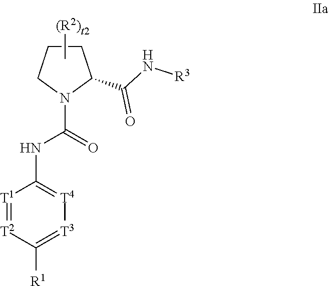

or a pharmaceutically acceptable salt thereof.

Embodiment 112. The method of embodiment 108, wherein the GIPR antagonist small molecule compound has the Formula III or IIIa:

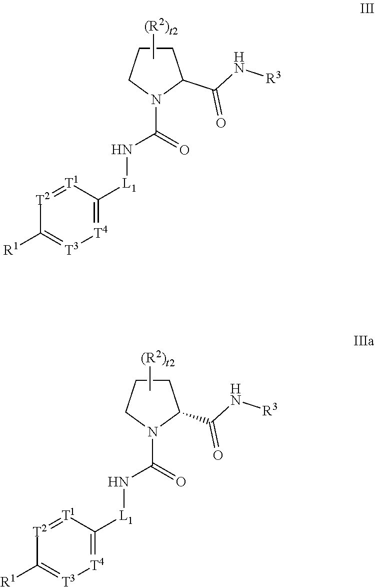

or a pharmaceutically acceptable salt thereof.

Embodiment 113. The method of embodiment 108, wherein the GIPR antagonist small molecule compound has the Formula IV or Formula IVa:

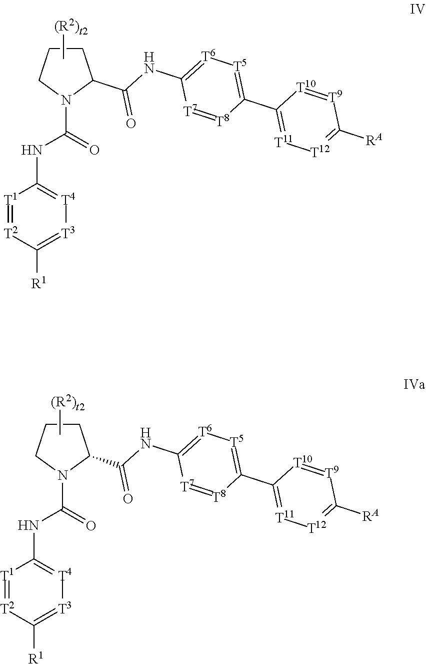

or a pharmaceutically acceptable salt thereof.

Embodiment 114. The method of embodiment 108, wherein the GIPR antagonist small molecule compound has the Formula V or Formula Va:

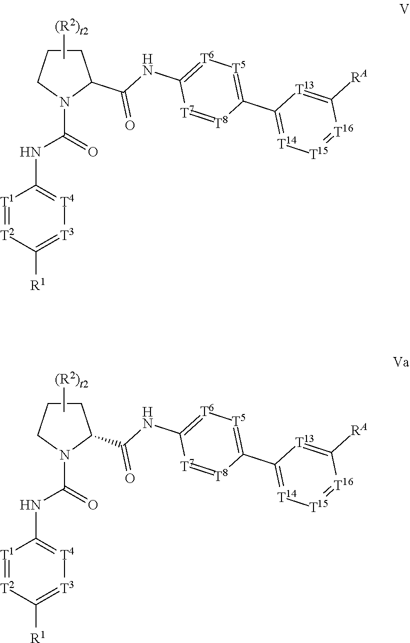

or a pharmaceutically acceptable salt thereof.

Embodiment 115. The method of embodiment 108, wherein the GIPR antagonist small molecule compound has the Formula VI or Formula VIa:

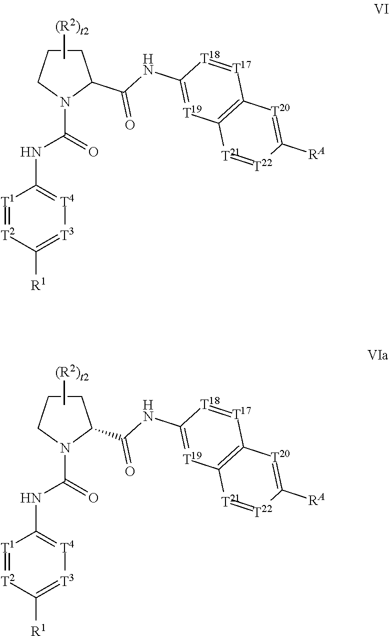

or a pharmaceutically acceptable salt thereof.

Embodiment 116. The method of embodiment 108, wherein the GIPR antagonist small molecule compound has the Formula VII or Formula VIIa:

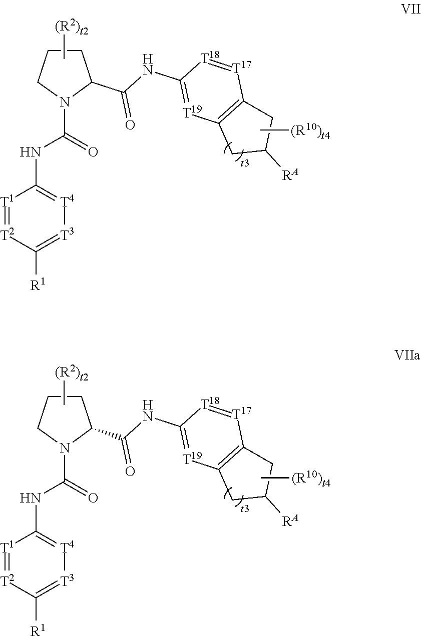

or a pharmaceutically acceptable salt thereof.

Embodiment 117. The method of any one of embodiments 108 to 116, wherein R¹ is cyclopropyl, cyclobutyl, R^{1a}, R^{1b}, or R^{1c},

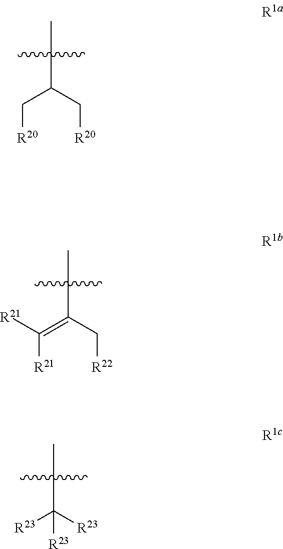

wherein each of the cyclopropyl or cyclobutyl is optionally substituted with 1, 2, 3, or 4 R^{S};
each R²⁰ is independently H, halogen, —OH, C₁₋₂ alkyl, C₁₋₂ haloalkyl, C₁₋₂ alkoxy, or C₁₋₂ haloalkoxy;
each R²¹ is independently H, C₁₋₂ alkyl, or C₁₋₂ haloalkyl;
R²² is H, halogen, C₁₋₂ alkyl, C₁₋₂ hydroxylalkyl, C₁₋₂ haloalkyl, C₁₋₂ alkoxy, or C₁₋₂ haloalkoxy;
each R²³ is independently halogen, C₁₋₂ alkyl, C₁₋₂ hydroxylalkyl, C₁₋₂ haloalkyl, C₁₋₂ alkoxy, or C₁₋₂ haloalkoxy; and
each R^{S} is independently halogen, —OH, C₁₋₂ alkyl, C₁₋₂ hydroxylalkyl, C₁₋₂ haloalkyl, C₁₋₂ alkoxy, or C₁₋₂ haloalkoxy.

Embodiment 118. The method of any one of embodiments 108 to 117, wherein R¹ is propan-2-yl, prop-1-en-2-yl, or cyclopropyl.

Embodiment 119. The method of any one of embodiments 108 to 118, wherein R¹ is propan-2-yl.

Embodiment 120. The method of any one of embodiments 108 to 119, wherein each of T¹, T², T³, and T⁴ is independently CR⁴.

Embodiment 121. The method of any one of embodiments 108 to 119, wherein one of T¹, T², T³, and T⁴ is N, and the other three are each independently CR⁴.

Embodiment 122. The method of any one of embodiments 108 to 121, wherein each R² is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-; and t2 is 0, 1, or 2.

Embodiment 123. The method of any one of embodiments 108-114 and 117-122, wherein each of T⁵, T⁶, T⁷, and T⁸ is independently CR⁵.

Embodiment 124. The method of any one of embodiments 108-114 and 117-122, wherein one of T⁵, T⁶, T⁷, and T⁸ is N and the other three are each independently CR⁵.

Embodiment 125. The method of any one of embodiments 108-113 and 117-124, wherein each of T⁹, T¹⁰, T¹¹, and T¹² is independently CR⁶.

Embodiment 126. The method of any one of embodiments 108-113 and 117-124, wherein one of T⁹, T¹⁰, T¹¹, and T¹² is N and the other three are each independently CR⁶.

Embodiment 127. The method of any one of embodiments 108-112, 114, and 117-124, wherein each of T¹³, T¹⁴, T¹⁵, and T¹⁶ is independently CR⁷.

Embodiment 128. The method of any one of embodiments 108-112, 114, and 117-122, wherein one of T¹³, T¹⁴, T¹⁵, and T¹⁶ is N and the other three are each independently CR⁷.

Embodiment 129. The method of any one of embodiments 108-112 and 115-122, wherein each of T¹⁷, T¹⁸, and T¹⁹ is independently CR³.

Embodiment 130. The method of any one of embodiments 108-112 and 115-122, wherein one of T¹⁷, T¹⁸, and T¹⁹ is N, and the other two are each independently CR³.

Embodiment 131. The method of any one of embodiments 108-112, 115, 117-122, 129, and 130, wherein each of T²⁰, T²¹, and T²² is independently CR⁹.

Embodiment 132. The method of any one of embodiments 108-113, 115, 117-122, 129, and 130, wherein one of T²⁰, T²¹, and T²² is N, and the other two are each independently CR⁹.

Embodiment 133. The method of any one of embodiments 108-113, 116, 117-122, 129, and 130, wherein t3 is 2.

Embodiment 134. The method of any one of embodiments 108-113, 115, 117-122, 129, 130, and 133, wherein t4 is 0, 1, or 2; and each R¹⁰ is independently halogen, —OH, C₁₋₄ alkyl, C₁₋₄ hydroxylalkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-.

Embodiment 135. The method of any one of embodiments 108-134, wherein R^{A} is —C(═O)—OH.

Embodiment 136. The method of embodiment 108, wherein the GIPR antagonist small molecule compound is selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid; and
3′,5′-difluoro-4′-[(1-{[4-(propan-2-yl)phenyl]carbamoyl}-DL-prolyl)amino][1,1′-biphenyl]-4-carboxylic acid,
   or a pharmaceutically acceptable salt thereof.

Embodiment 137. The method of embodiment 108, wherein the GIPR antagonist small molecule compound is selected from the group consisting of:
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid; and
3′,5′-difluoro-4′-[(1-{[4-(propan-2-yl)phenyl]carbamoyl}-D-prolyl)amino][1,1′-biphenyl]-4-carboxylic acid,
   or a pharmaceutically acceptable salt thereof.

Embodiment 138. The method of embodiment 108, wherein the GIPR antagonist small molecule compound selected from:
4′-[(1-{[4-(propan-2-yl)phenyl]carbamoyl}-DL-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid; and
6-methyl-5-{4-[(1-{[3-methyl-4-(trifluoromethyl)phenyl]carbamoyl}-DL-prolyl)amino]phenyl}pyridine-2-carboxylic acid,

or a pharmaceutically acceptable salt thereof.

Embodiment 139. The method of embodiment 108, wherein the GIPR antagonist small molecule compound is selected from the group consisting of:
4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid; and
6-methyl-5-{4-[(1-{[3-methyl-4-(trifluoromethyl)phenyl]carbamoyl}-D-prolyl)amino]phenyl}pyridine-2-carboxylic acid,

or a pharmaceutically acceptable salt thereof.

Embodiment 140. The method of embodiment 108, wherein the GIPR antagonist small molecule compound is selected from the group consisting of:
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid; and
3-[6-({1-[(4-cyclobutylphenyl)carbamoyl]-DL-prolyl}amino)pyridin-3-yl]benzoic acid,

or a pharmaceutically acceptable salt thereof.

Embodiment 141. The method of embodiment 108, wherein the GIPR antagonist small molecule compound is selected from the group consisting of:
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof.

Embodiment 142. The method of any one of the preceding embodiments, wherein the condition is obesity.

Embodiment 143. The method of any one of the preceding embodiments, wherein the condition is diabetes.

Embodiment 144. The method of any of the preceding embodiments, wherein the therapeutically effective amount of the GIPR antagonist small molecule compound is from about 1 mg to about 100 mg.

Embodiment 145. The method of any of the preceding embodiments, wherein the therapeutically effective amount of the GIPR antagonist small molecule compound is from about 1 mg to about 25 mg.

Embodiment 146. The method of any of the preceding embodiments, wherein the GIPR antagonist small molecule compound or a pharmaceutically acceptable salt thereof is administered orally.

Embodiment 147. The method of any of the preceding embodiments, wherein the GIPR antagonist small molecule compound or a pharmaceutically acceptable salt thereof is administered by subcutaneous injection.

Embodiment 148. The method of any one of the preceding embodiments, wherein the GIPR antagonist small molecule compound or a pharmaceutically acceptable salt thereof is administered daily.

Embodiment 149. The method of any one of the preceding embodiments, wherein the GIPR antagonist small molecule compound or a pharmaceutically acceptable salt thereof is administered weekly.

Embodiment 150. The method of any one of embodiments 108-149, wherein the GLP-1R agonist small molecule compound is a compound of Formula D-I:

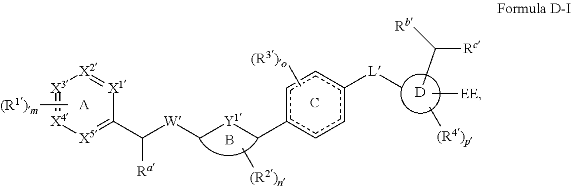

or a pharmaceutically acceptable salt thereof,
wherein:
   [symbol] indicates a single bond or a double bond;
   X^{1′}, X^{2′}, X^{3′}, X^{4′}, and X^{5′} are each independently selected from N and CH;
   W is selected from O, S, CR^{5′}R^{6′}, and NR^{5′a};
   ring B is 6-membered heteroaryl, 6-membered monocyclic heterocyclyl, or phenyl, wherein Y″ is selected from N, NH, CH, and CH₂;
   ring C is cyclohexyl, phenyl, or pyridyl;
   L′ is CHR^{d′}, O, S, or NR^{5′a};
   ring D is bicyclic heteroaryl;
   EE is —COOH or a carboxylic group surrogate, optionally, the carboxylic group surrogate is:

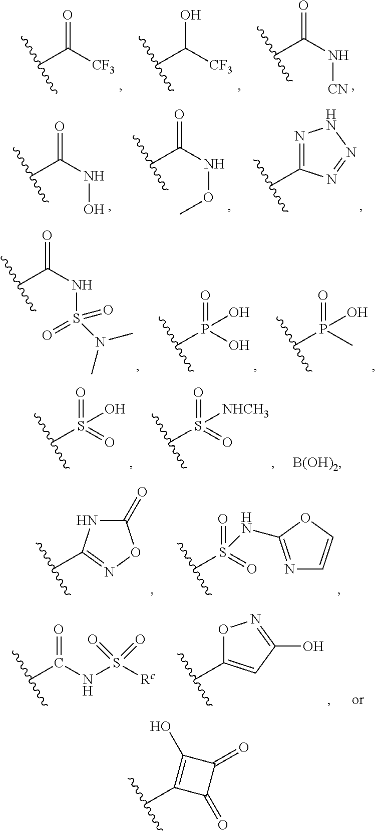

each R^{a′} and R^{b′} are independently selected from hydrogen, deuterium, halogen, —CN, C₁-C₆alkyl, C₁-C₆alkoxy, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆alkyl or C₁-C₆alkoxy represented by R^{a′}/R^{b′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{a′}/R^{b′} or in the group represented by R^{a′}/R^{b′} is optionally substituted with one or more groups selected from halogen, oxo, CN, OH, C₁-C₃ alkyl (optionally substituted with 1 to 3 groups selected from F, OH, and —OCH₃), and C₁-C₃ alkoxy (optionally substituted with 1 to 3 groups selected from F, OH, and —OCH₃), and NR^{5′a}R^{6′a};
each R^{c′} and R^{d′} are independently selected from hydrogen, deuterium, halogen, —CN, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{c′}/R^{d′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{c′}/R^{d′} or in the group represented by R^{c′}/R^{d′} is optionally substituted with one or more groups selected from halogen, oxo, CN, and NR^{5′a}R^{6′a};
each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{2′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{2′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{2′} or in the group represented by R^{2′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{3′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{3′} or in the group represented by R^{3′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆ alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃);
R^{5′} and R^{6′} are each independently selected from hydrogen, deuterium, halogen, CN, OH, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{5′} or R^{6′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{5′} or R^{6′} or in the group represented by R^{5′} or R^{6′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
R^{5′a} and R^{6′a} are each independently selected from hydrogen and C₁-C₆ alkyl;
wherein optionally two R^{1′}; two R^{2′}; two R^{3′}; two R^{4′}; R^{1′} and R^{2′}; R^{2′} and R^{3′}; R^{a′} and R^{1′}; R^{a′} and R^{2′}; R^{1′} and any of R^{5′}, R^{5′a} (in the group represented by W) or R^{6′}; R^{a′} and any of R^{5′}, R^{5′a}(in the group represented by W) or R^{6′}; R^{2′} and any of R^{5′}, R^{5′a} (in the group represented by W) or R^{6′}; R^{5′} and R^{6′}; any of two groups selected from R^{c′}, R^{d′}, R^{e′}, and R^{f′}; or R^{4′} and any one of R^{c′}, R^{d′}, R^{e′}, and R^{f′}; taken together with their respective intervening carbon or hetero atom(s), form phenyl, 5-6 membered heteroaryl, 4-8 membered saturated or partially saturated cycloalkyl or 4-8 membered saturated or partially saturated heterocyclyl, and each of which is optionally substituted with one or more groups selected from halogen, —CN, —OH, CF₃, C₁-C₆ alkyl, C₁-C₆ alkoxy, —NH₂, —NHC₁-C₆ alkyl, —N(C₁-C₆ alkyl)₂, oxo, and saturated or partially saturated C₃-C₆ cycloalkyl, wherein the C₁-C₆ alkyl and C₁-C₆ alkoxy is optionally substituted with one or more groups selected from halogen, oxo, CN, CF₃, OH, OCH₃, OCH₂CH₃, and saturated or partially saturated C₃-C₆ cycloalkyl, wherein the cycloalkyl is optionally substituted with one or more groups selected from halogen, oxo, CN, CF₃, OH, OCH₃, OCH₂CH₃;
m′ is an integer selected from 0, 1, 2, 3, and 4;
n′ is an integer selected from 0, 1, 2, 3, 4, and 5;
o′ is an integer selected from 0, 1, 2, 3, and 4; and
p′ is an integer selected from 0, 1, 2, 3, and 4.

Embodiment 151. The method of any one of embodiments 105-147, wherein the GLP-1R agonist small molecule compound is a compound of Formula D-II:

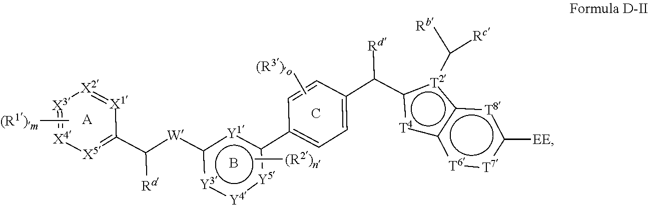

or a pharmaceutically acceptable salt thereof,
wherein:
   X^{1′}, X^{2′}, X^{3′}, X^{4′}, and X^{5′} are each independently selected from N and CH; wherein no more than three of X^{1′}, X^{2′}, X^{3′}, X^{4′}, and X^{5′} are N, and wherein ring A does not contain 3 nitrogen ring atoms at 3 contiguous positions;
   ring B is 6-membered heteroaryl or phenyl, wherein Y^{1′}, Y^{3′}, Y^{4′}, and Y^{6′} are each independently selected from N or CH; wherein there are no more than 3 nitrogen ring atoms in ring B, and wherein ring B does not contain 3 nitrogen ring atoms at 3 contiguous positions;
   T^{2′} is selected from N and C;
   T^{4′} is selected from N, NR^{4′}, O, S, and CR^{4′};
   T^{6′}, T^{7′}, and T^{8′} are each independently selected from N and CR^{4′};
   wherein no more than 4 of T^{2′}, T^{4′}, T^{6′}, T^{7′}, and T^{8′} are selected from N, O, and S.
   In one embodiment, the GLP-1R agonist is a compound of Formula D-I or D-II, or a pharmaceutically acceptable salt thereof, wherein:
   W is O, NH, or CH₂;
   R^{a′} is H, CH₃, or CF₃;
   R^{b′} is selected from hydrogen, C₁-C₆ alkyl, C₁-C₆ alkoxy, NR^{5′a}R^{6′a}, 6-10 membered aryl, 5-6 membered heteroaryl, 3-6 membered saturated or partially saturated cycloalkyl and 3-7 membered saturated or partially saturated heterocyclyl, wherein the C₁-C₆ alkyl or C₁-C₆ alkoxy represented by R^{b′} is optionally substituted with one or more groups selected from halogen, oxo, CN, OH, and C₃—C saturated or partially saturated cycloalkyl; and wherein the aryl, heteroaryl, saturated or partially saturated cycloalkyl, or saturated or partially saturated heterocyclyl represented by R^{b′} or in the group represented by R^{b′} is optionally substituted with one or more groups selected from halogen, oxo (when R^{b′} is non-aromatic), CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
   R^{c′} is selected from hydrogen, halogen, and C₁-C₄ alkyl optionally substituted with one or more groups selected from halogen and hydroxy;
   R^{d′} is H, F, CH₃, or CF₃; and
   each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo (when R^{1′} is non-aromatic), CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
   each R^{2′} and R^{3′} are independently selected from H, deuterium, halogen, —CN, OH, oxo, C₁-C₆ alkyl, C₁-C₆ alkoxy, NR^{5′a}R^{6′a}, wherein the C₁-C₆ alkyl or C₁-C₆ alkoxy represented by R^{2′} and/or R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃);
   each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃); and
   o′ is an integer selected from 0, 1, 2, 3, and 4.

Embodiment 152. The method of embodiment 151, wherein the GLP-1R agonist small molecule compound is a compound of Formula D-III:

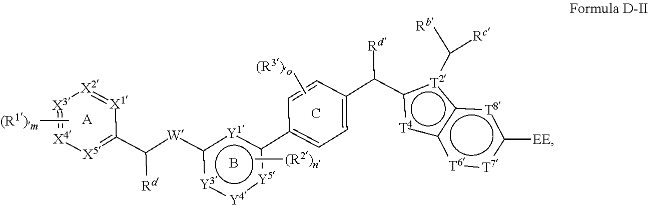

or a pharmaceutically acceptable salt, stereoisomer, solvate, or hydrate thereof, wherein R^{4′} is H, F, Cl, methyl, or methoxy.

Embodiment 153. A method of embodiment 150, wherein the GLP-1R agonist small molecule compound is selected from the group consisting of:
(S)-2-(4-(6-((4-chloro-2-fluorobenzyl)oxy)pyridin-2-yl)-3-fluorobenzyl)-1-(oxetan-2-ylmethyl)-1H-benzo[d]imidazole-6-carboxylic acid;
(S)-2-((3′-((4-chloro-2-fluorobenzyl)oxy)-2,4′-difluoro-[1,1′-biphenyl]-4-yl)methyl)-1-(oxetan-2-ylmethyl)-1H-benzo[d]imidazole-6-carboxylic acid;
(S)-2-(4-(4-(4-chloro-2-fluorobenzyloxy)-5-fluoropyrimidin-2-yl)-3-fluorobenzyl)-1-(oxetan-2-ylmethyl)-1H-benzo[d]imidazole-6-carboxylic acid;
(S)-2-((4-(3-(4-chloro-2-fluorobenzyloxy)isothiazol-4-yl)-5,6-dihydropyridin-1(2H)-yl)methyl)-1-(oxetan-2-ylmethyl)-1H-benzo[d]imidazole-6-carboxylic acid;
(S)-2-((1-(6-(4-cyano-2-fluorobenzyloxy)pyridin-2-yl)piperidin-4-ylidene)methyl)-3-(oxetan-2-ylmethyl)-3H-imidazo[4,5-b]pyridine-5-carboxylic acid;
(S)-2-(4-(6-((3,5-difluoropyridin-2-yl)methoxy)pyridin-2-yl)-2,5-difluorobenzyl)-3-(oxetan-2-ylmethyl)-3H-imidazo[4,5-b]pyridine-5-carboxylic acid;
(S)-2-(4-(4-((2,4-difluorobenzyl)oxy)-5-fluoropyrimidin-2-yl)-2-fluorobenzyl)-3-(oxetan-2-ylmethyl)-3H-imidazo[4,5-b]pyridine-5-carboxylic acid;
(S)-2-(4-(6-((4-cyano-2-fluorobenzyl)oxy)pyridin-2-yl)-2,5-difluorobenzyl)-1-(oxetan-2-ylmethyl)-1H-benzo[d]imidazole-6-carboxylic acid;
(S)-2-(2-chloro-4-(6-((4-chloro-2-fluorobenzyl)oxy)pyridin-2-yl)benzyl)-1-(oxetan-2-ylmethyl)-1H-benzo[d]imidazole-6-carboxylic acid;
(S)-2-((4-(2-((4-chloro-2-fluorobenzyl)oxy)oxazol-4-yl)-5,6-dihydropyridin-1(2H)-yl)methyl)-1-(oxetan-2-ylmethyl)-1H-benzo[d]imidazole-6-carboxylic acid; and
(S)-2-(4-(6-((4-chloro-2-fluorobenzyl)oxy)pyridin-2-yl)benzyl)-1-(oxetan-2-ylmethyl)-1H-benzo[d]imidazole-6-carboxylic acid,
   or a pharmaceutically acceptable salt thereof.

Embodiment 154. The method of any one of embodiments 108-149, wherein the GLP-1R agonist small molecule compound is a compound of Formula E-I:

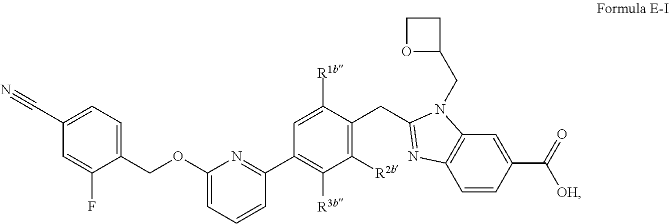

or a pharmaceutically acceptable salt thereof,
wherein:
   R^{1b″} is H or F
   R^{2b″} is H or F; and
   R^{3b″} is H or CH₃.

Embodiment 155. The method of embodiment 154, wherein the GLP-1R agonist small molecule compound is a compound of Formula E-II:

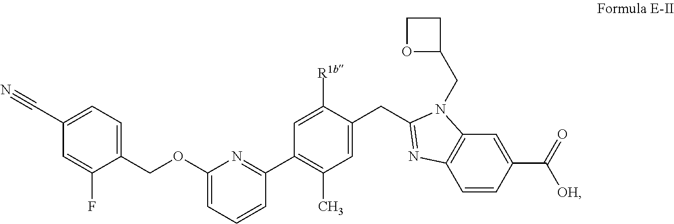

or a pharmaceutically acceptable salt thereof.

Embodiment 156. The method of embodiment 154, wherein the GLP-1R agonist small molecule compound is a compound of Formula E-III:

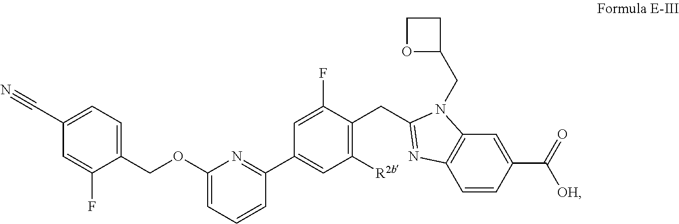

or a pharmaceutically acceptable salt thereof, wherein R^{2″} is H or F

Embodiment 157. The method of embodiment 154, wherein the GLP-1R agonist small molecule compound is selected from the group consisting of:
2-[[4-[6-[(4-cyano-2-fluoro-phenyl)methoxy]-2-pyridyl]-3-methyl-phenyl]methyl]-3-[[oxetan-2-yl]methyl]benzimidazole-5-carboxylic acid;
2-[[4-[6-[(4-cyano-2-fluoro-phenyl)methoxy]-2-pyridyl]-2-fluoro-5-methyl-phenyl]methyl]-3-[[oxetan-2-yl]methyl]benzimidazole-5-carboxylic acid;
2-[[4-[6-[(4-Cyano-2-fluoro-phenyl)methoxy]-2-pyridyl]-2-fluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2,6-difluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2-fluoro-3-methyl-phenyl]methyl]-3-[[(2S)-oxetan-2-yl]methyl]benzimidazole-5-carboxylic acid; and
2-[[4-[6-[(4-Cyano-2-fluoro-phenyl)methoxy]-2-pyridyl]phenyl]methyl]-3-[[(2S)-oxetan-2-yl]methyl]benzimidazole-5-carboxylic acid,

or a pharmaceutically acceptable salt thereof.

Embodiment 158. The method of any one of embodiments 108-149, wherein the GLP-1R agonist small molecule compound is orforglipron, or a pharmaceutically acceptable salt thereof.

Embodiment 159. The method of any one of embodiments 108-149, wherein the GLP-1R agonist small molecule compound is:

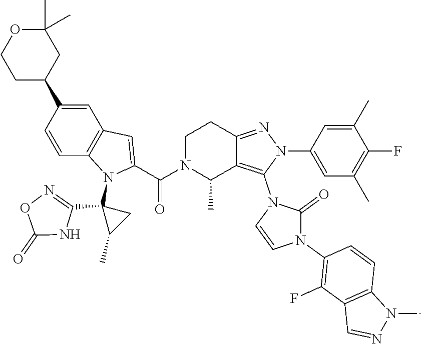

Embodiment 160. A method of treating a disease or condition comprising administering to a subject in need thereof a therapeutically effective amount of:
c) a GIPR antagonist small molecule compound or a pharmaceutically acceptable salt thereof selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,
   or a pharmaceutically acceptable salt thereof; and
   d) a GLP-1R agonist small molecule compound selected from the group consisting of:
(S)-2-(4-(6-((4-chloro-2-fluorobenzyl)oxy)pyridin-2-yl)-3-fluorobenzyl)-1-(oxetan-2-ylmethyl)-1H-benzo[d]imidazole-6-carboxylic acid;
(S)-2-((3′-((4-chloro-2-fluorobenzyl)oxy)-2,4′-difluoro-[1,1′-biphenyl]-4-yl)methyl)-1-(oxetan-2-ylmethyl)-1H-benzo[d]imidazole-6-carboxylic acid;
(S)-2-(4-(4-(4-chloro-2-fluorobenzyloxy)-5-fluoropyrimidin-2-yl)-3-fluorobenzyl)-1-(oxetan-2-ylmethyl)-1H-benzo[d]imidazole-6-carboxylic acid;
(S)-2-((4-(3-(4-chloro-2-fluorobenzyloxy)isothiazol-4-yl)-5,6-dihydropyridin-1(2H)-yl)methyl)-1-(oxetan-2-ylmethyl)-1H-benzo[d]imidazole-6-carboxylic acid;
(S)-2-((1-(6-(4-cyano-2-fluorobenzyloxy)pyridin-2-yl)piperidin-4-ylidene)methyl)-3-(oxetan-2-ylmethyl)-3H-imidazo[4,5-b]pyridine-5-carboxylic acid;
(S)-2-(4-(6-((3,5-difluoropyridin-2-yl)methoxy)pyridin-2-yl)-2,5-difluorobenzyl)-3-(oxetan-2-ylmethyl)-3H-imidazo[4,5-b]pyridine-5-carboxylic acid;
(S)-2-(4-(4-((2,4-difluorobenzyl)oxy)-5-fluoropyrimidin-2-yl)-2-fluorobenzyl)-3-(oxetan-2-ylmethyl)-3H-imidazo[4,5-b]pyridine-5-carboxylic acid;
(S)-2-(4-(6-((4-cyano-2-fluorobenzyl)oxy)pyridin-2-yl)-2,5-difluorobenzyl)-1-(oxetan-2-ylmethyl)-1H-benzo[d]imidazole-6-carboxylic acid;
(S)-2-(2-chloro-4-(6-((4-chloro-2-fluorobenzyl)oxy)pyridin-2-yl)benzyl)-1-(oxetan-2-ylmethyl)-1H-benzo[d]imidazole-6-carboxylic acid;
(S)-2-((4-(2-((4-chloro-2-fluorobenzyl)oxy)oxazol-4-yl)-5,6-dihydropyridin-1(2H)-yl)methyl)-1-(oxetan-2-ylmethyl)-1H-benzo[d]imidazole-6-carboxylic acid; and
(S)-2-(4-(6-((4-chloro-2-fluorobenzyl)oxy)pyridin-2-yl)benzyl)-1-(oxetan-2-ylmethyl)-1H-benzo[d]imidazole-6-carboxylic acid,
   or a pharmaceutically acceptable salt thereof.

Embodiment 161. A method of treating a disease or condition comprising administering to a subject in need thereof a therapeutically effective amount of:
c) a GIPR antagonist small molecule compound or a pharmaceutically acceptable salt thereof selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof; and
d) a GLP-1R agonist small molecule compound selected from the group consisting of:
2-[[4-[6-[(4-cyano-2-fluoro-phenyl)methoxy]-2-pyridyl]-3-methyl-phenyl]methyl]-3-[[oxetan-2-yl]methyl]benzimidazole-5-carboxylic acid;
2-[[4-[6-[(4-cyano-2-fluoro-phenyl)methoxy]-2-pyridyl]-2-fluoro-5-methyl-phenyl]methyl]-3-[[oxetan-2-yl]methyl]benzimidazole-5-carboxylic acid;
2-[[4-[6-[(4-Cyano-2-fluoro-phenyl)methoxy]-2-pyridyl]-2-fluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2,6-difluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2-fluoro-3-methyl-phenyl]methyl]-3-[[(2S)-oxetan-2-yl]methyl]benzimidazole-5-carboxylic acid; and
2-[[4-[6-[(4-Cyano-2-fluoro-phenyl)methoxy]-2-pyridyl]phenyl]methyl]-3-[[(2S)-oxetan-2-yl]methyl]benzimidazole-5-carboxylic acid,

or a pharmaceutically acceptable salt thereof.

Embodiment 162. The method of any one of embodiments 108-161, wherein the GLP-1R agonist small molecule compound or pharmaceutically acceptable salt thereof is administered orally.

Embodiment 163. The method of any one of embodiments 108-161, wherein the GLP-1R agonist small molecule compound or pharmaceutically acceptable salt thereof is administered by subcutaneous injection.

Embodiment 164. The method of any one of embodiments 108-163, wherein the GLP-1R agonist small molecule compound or pharmaceutically acceptable salt thereof is administered once a day.

Embodiment 165. The method of any one of embodiments 108-163, wherein the GLP-1R agonist small molecule compound or pharmaceutically acceptable salt thereof is administered once a week.

Embodiment 166. The method of any one of embodiments 108-163, wherein the GLP-1R agonist small molecule compound or pharmaceutically acceptable salt thereof is administered once every two weeks.

Embodiment 167. The method of any one of embodiments 108-166, wherein the GIPR antagonist small molecule compound or pharmaceutically acceptable salt thereof is administered orally.

Embodiment 168. The method of any one of embodiments 108-166, wherein the GIPR antagonist small molecule compound or pharmaceutically acceptable salt thereof is administered by subcutaneous injection.

Embodiment 169. The method of any one of embodiments 108-168, wherein the GIPR antagonist small molecule compound or pharmaceutically acceptable salt thereof is administered once a day.

Embodiment 170. The method of any one of embodiments 108-168, wherein the GIPR antagonist small molecule compound or pharmaceutically acceptable salt thereof is administered once a week.

Embodiment 171. The method of any one of embodiments 108-168, wherein the GIPR antagonist small molecule compound or pharmaceutically acceptable salt thereof is administered once every two weeks.

Embodiment 172. The method of any of the preceding embodiments, wherein the subject is human.

Embodiment 173. The method of embodiment 172, wherein the subject has a Body Mass Index of at least about 27 kg/m².

Embodiment 174. The method of embodiment 172, wherein the subject has a body mass index of at least about 27 kg/m² and has a weight-related health condition.

Embodiment 175. The method of embodiment 174, wherein the weight-related condition is selected from the group consisting of type 2 diabetes, insulin resistance, polycystic ovary syndrome, hypertension, sleep apnea, and high cholesterol.

Embodiment 176. A pharmaceutical composition comprising:
a) a GIPR antagonist small molecule compound of Formula I:

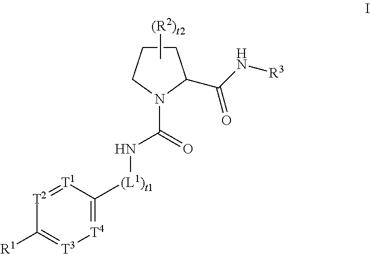

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

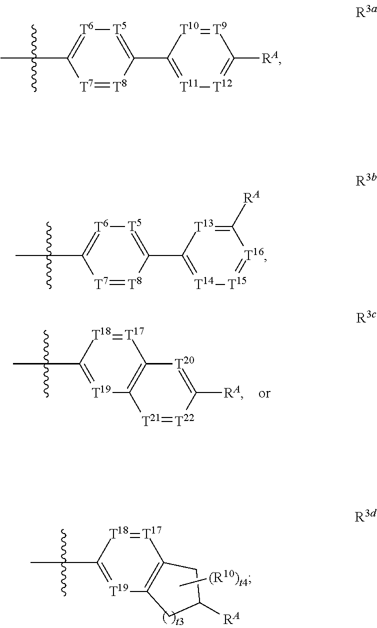

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula D-I:

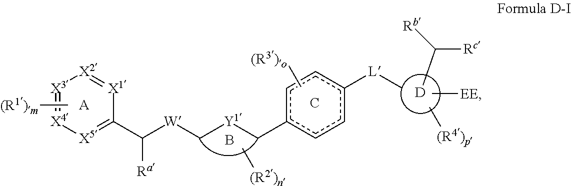

or a pharmaceutically acceptable salt thereof,
wherein:
   [symbol] indicates a single bond or a double bond;
   X^{1′}, X^{2′}, X^{3′}, X^{4′}, and X^{5′} are each independently selected from N and CH;
   W is selected from O, S, CR^{5′}R^{6′}, and NR^{5′a};
   ring B is 6-membered heteroaryl, 6-membered monocyclic heterocyclyl, or phenyl, wherein Y″ is selected from N, NH, CH, and CH₂;
   ring C is cyclohexyl, phenyl, or pyridyl;
   L′ is CHR^{d′}, O, S, or NR^{5′a};
   ring D is bicyclic heteroaryl;
   EE is —COOH or a carboxylic group surrogate, optionally, the carboxylic group surrogate is:

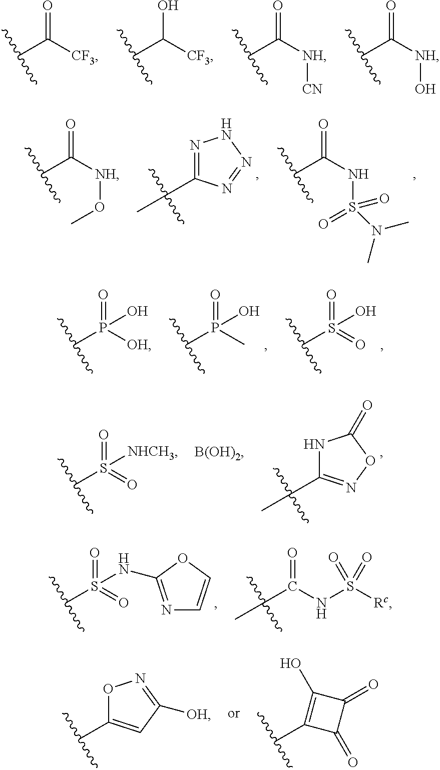

each R^{a′} and R^{b′} are independently selected from hydrogen, deuterium, halogen, —CN, C₁-C₆alkyl, C₁-C₆alkoxy, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆alkyl or C₁-C₆alkoxy represented by R^{a′}/R^{b′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{a′}/R^{b′} or in the group represented by R^{a′}/R^{b′} is optionally substituted with one or more groups selected from halogen, oxo, CN, OH, C₁-C₃ alkyl (optionally substituted with 1 to 3 groups selected from F, OH, and —OCH₃), and C₁-C₃ alkoxy (optionally substituted with 1 to 3 groups selected from F, OH, and —OCH₃), and NR^{5′a}R^{6′a};
each R^{c′} and R^{d′} are independently selected from hydrogen, deuterium, halogen, —CN, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{c′}/R^{d′} is optionally substituted with one or more groups selected from halogen, oxo, CN, OH, and C₃-C₆ saturated or partially saturated cycloalkyl; and wherein the aryl, heteroaryl, saturated or partially saturated cycloalkyl, or saturated or partially saturated heterocyclyl represented by R^{c′}/R^{d′} or in the group represented by R^{c′}/R^{d′} is optionally substituted with one or more groups selected from halogen, oxo, CN, and NR^{5′a}R^{6′a};
each R^{1′} is independently selected from H, deuterium, halogen, —CN, OH, C₁-C₆ alkyl, C₁-C₆ alkoxy, C₂-C₆ alkenyl, C₂-C₆ alkynyl, NR^{5′a}R^{6′a}, 6-10 membered aryl, 5-8 membered heteroaryl, 3-8 membered saturated or partially saturated cycloalkyl, and 3-8 membered saturated or partially saturated heterocyclyl, wherein the C₁-C₆ alkyl, C₁-C₆ alkoxy, C₂-C₆ alkenyl, or C₂-C₆ alkynyl represented by R^{1′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{1′} or in the group represented by R^{1′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{2′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{2′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{2′} or in the group represented by R^{2′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{3′} is independently selected from H, deuterium, halogen, —CN, OH, oxo, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{3′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{3′} or in the group represented by R^{3′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
each R^{4′} is independently selected from H, deuterium, halogen, OH, —CN, C₁-C₆ alkyl, C₁-C₆ alkoxy, and NR^{5′a}R^{6′a}, wherein the C₁-C₆ alkyl and C₁-C₆ alkoxy represented by R^{4′} is optionally substituted with one or more groups selected from halogen, oxo, CN, CF₃, and saturated or partially saturated C₃-C₆ cycloalkyl (optionally substituted with one or more groups selected from halogen, oxo, CN, CF₃, OH, OCH₃, OCH₂CH₃);
R^{5′} and R^{6′} are each independently selected from hydrogen, deuterium, halogen, CN, OH, C₁-C₆ alkyl, C₁-C₆ alkoxy, NR^{5′a}R^{6′a}, 6-10 membered aryl, 5-8 membered heteroaryl, 3-8 membered saturated or partially saturated cycloalkyl and 3-8 membered saturated or partially saturated heterocyclyl, wherein the C₁-C₆ alkyl or C₁-C₆ alkoxy represented by R^{5′} or R^{6′} is optionally substituted with one or more groups selected from halogen, oxo, CN, CF₃, OH, OCH₃, OCH₂CH₃, and saturated or partially saturated C₃-C₆ cycloalkyl (optionally substituted with one or more groups selected from halogen, oxo, CN, CF₃, OH, OCH₃, OCH₂CH₃); and wherein the aryl, heteroaryl, saturated or partially saturated cycloalkyl, or saturated or partially saturated heterocyclyl represented by R^{5′} or R^{6′} or in the group represented by R^{5′} or R^{6′} is optionally substituted with one or more groups selected from halogen, oxo, CN, OH, C₁-C₃ alkyl (optionally substituted with 1 to 3 groups selected from F, OH, and OCH₃), and C₁-C₃ alkoxy (optionally substituted with 1 to 3 groups selected from F, OH, and OCH₃), and NR^{5′a}R^{6′a};
R^{5′a} and R^{6′a} are each independently selected from hydrogen and C₁-C₆ alkyl;
wherein optionally two R^{1′}; two R^{2′}; two R^{3′}; two R^{4′}; R^{1′} and R^{2′}; R^{2′} and R^{3′}; R^{a′} and R^{1′}; R^{a′} and R^{2′}; R^{1′} and any of R^{5′}, R^{5′a} (in the group represented by W) or R^{6′}; R^{a′} and any of R^{5′}, R^{5′a} (in the group represented by W) or R^{6′}; R^{2′} and any of R^{5′}, R^{5′} (in the group represented by W) or R^{6′}; R^{5′} and R^{6′}; any of two groups selected from R^{c′}, R^{d′}, R^{e′}, and R^{f′}; or R^{4′} and any one of R^{c′}, R^{d′}, R^{e′}, and R^{f′}; taken together with their respective intervening carbon or hetero atom(s), form phenyl, 5-6 membered heteroaryl, 4-8 membered saturated or partially saturated cycloalkyl or 4-8 membered saturated or partially saturated heterocyclyl, and each of which is optionally substituted with one or more groups selected from halogen, —CN, —OH, CF₃, C₁-C₆ alkyl, C₁-C₆ alkoxy, —NH₂, —NHC₁-C₆ alkyl, —N(C₁-C₆ alkyl)₂, oxo, and saturated or partially saturated C₃-C₆ cycloalkyl, wherein the C₁-C₆ alkyl and C₁-C₆ alkoxy is optionally substituted with one or more groups selected from halogen, oxo, CN, CF₃, OH, OCH₃, OCH₂CH₃, and saturated or partially saturated C₃-C₆ cycloalkyl, wherein the cycloalkyl is optionally substituted with one or more groups selected from halogen, oxo, CN, CF₃, OH, OCH₃, OCH₂CH₃;
m′ is an integer selected from 0, 1, 2, 3, and 4;
n′ is an integer selected from 0, 1, 2, 3, 4, and 5;
o′ is an integer selected from 0, 1, 2, 3, and 4; and
p′ is an integer selected from 0, 1, 2, 3, and 4.

Embodiment 177. A pharmaceutical composition comprising:
a) a GIPR antagonist small molecule compound of Formula I:

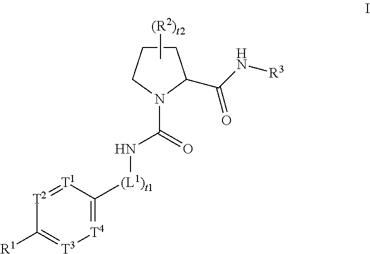

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

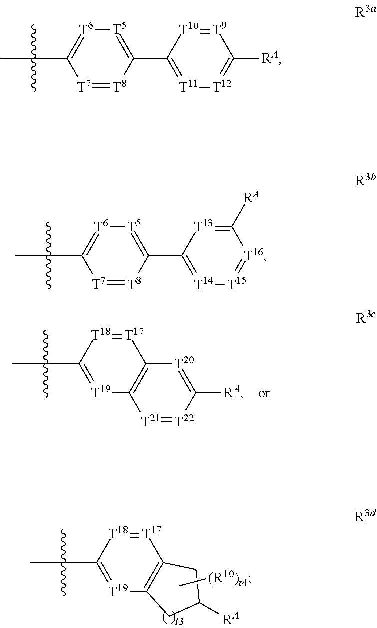

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L}₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula E-I:

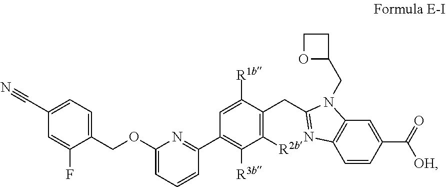

or a pharmaceutically acceptable salt thereof,
wherein:
   R^{1b″} is H or F
   R^{2b″} is H or F; and
   R^{3b″} is H or CH₃.

Embodiment 178. The pharmaceutical composition of embodiments 176 or 177, wherein the GIPR antagonist small molecule is selected from the group consisting of:
5-{4-[(1-{[4-(propan-2-yl)phenyl]carbamoyl}-DL-prolyl)amino]phenyl}pyridine-2-carboxylic acid;
6-{4-[(1-{[4-(propan-2-yl)phenyl]carbamoyl}-DL-prolyl)amino]phenyl}pyridine-3-carboxylic acid;
4′-[(1-{[4-(propan-2-yl)phenyl]carbamoyl}-DL-prolyl)amino][1,1′-biphenyl]-3-carboxylic acid;
4′-[(1-{[4-(propan-2-yl)phenyl]carbamoyl}-DL-prolyl)amino][1,1′-biphenyl]-4-carboxylic acid;
4-{6-[(1-{[4-(propan-2-yl)phenyl]carbamoyl}-DL-prolyl)amino]pyridin-3-yl}benzoic acid;
3′-fluoro-4′-[(1-{[4-(propan-2-yl)phenyl]carbamoyl}-DL-prolyl)amino][1,1′-biphenyl]-4-carboxylic acid;
4′-({1-[(4-cyclopropylphenyl)carbamoyl]-DL-prolyl}amino)[1,1′-biphenyl]-4-carboxylic acid;
2-{4-[(1-{[4-(propan-2-yl)phenyl]carbamoyl}-DL-prolyl)amino]phenyl}pyrimidine-5-carboxylic acid;
6-{4-[(1-{[4-(propan-2-yl)phenyl]carbamoyl}-DL-prolyl)amino]phenyl}pyridine-2-carboxylic acid;
6-[(1-{[4-(propan-2-yl)phenyl]carbamoyl}-DL-prolyl)amino]naphthalene-2-carboxylic acid;
8-methyl-6-[(1-{[4-(propan-2-yl)phenyl]carbamoyl}-DL-prolyl)amino]quinoline-2-carboxylic acid;
4′-[(1-{[4-(prop-1-en-2-yl)phenyl]carbamoyl}-DL-prolyl)amino][1,1′-biphenyl]-4-carboxylic acid;
4′-({1-[(4-chlorophenyl)carbamoyl]-DL-prolyl}amino)[1,1′-biphenyl]-4-carboxylic acid;
4-{4-[(1-{[4-(propan-2-yl)phenyl]carbamoyl}-DL-prolyl)amino]phenyl}pyridine-2-carboxylic acid;
3′,5′-difluoro-4′-[(1-{[4-(propan-2-yl)phenyl]carbamoyl}-DL-prolyl)amino][1,1′-biphenyl]-4-carboxylic acid;
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid;
3′,5′-difluoro-4′-[(1-{[4-(propan-2-yl)phenyl]carbamoyl}-D-prolyl)amino][1,1′-biphenyl]-4-carboxylic acid;
5-{4-[(1-{[3-methyl-4-(propan-2-yl)phenyl]carbamoyl}-DL-prolyl)amino]phenyl}pyridine-2-carboxylic acid;
6-methyl-5-{4-[(1-{[4-(propan-2-yl)phenyl]carbamoyl}-DL-prolyl)amino]phenyl}pyridine-2-carboxylic acid;
3-methoxy-4′-[(1-{[4-(propan-2-yl)phenyl]carbamoyl}-DL-prolyl)amino][1,1′-biphenyl]-4-carboxylic acid;
5-{4-[(1-{[3-fluoro-4-(propan-2-yl)phenyl]carbamoyl}-DL-prolyl)amino]phenyl}-6-methylpyridine-2-carboxylic acid;
3-fluoro-4′-[(1-{[4-(propan-2-yl)phenyl]carbamoyl}-DL-prolyl)amino][1,1′-biphenyl]-4-carboxylic acid;
4-{6-[(1-{[4-(trifluoromethyl)phenyl]carbamoyl}-DL-prolyl)amino]pyridin-3-yl}benzoic acid;
4′-[(1-{[3-fluoro-4-(propan-2-yl)phenyl]carbamoyl}-DL-prolyl)amino]-3-methoxy[1,1′-biphenyl]-4-carboxylic acid;
4-{3-fluoro-5-[(1-{[4-(propan-2-yl)phenyl]carbamoyl}-DL-prolyl)amino]pyridin-2-yl}benzoic acid;
6-methyl-5-{4-[(1-{[3-methyl-4-(trifluoromethyl)phenyl]carbamoyl}-DL-prolyl)amino]phenyl}pyridine-2-carboxylic acid;
5-{4-[(1-{[3-methyl-4-(propan-2-yl)phenyl]carbamoyl}-D-prolyl)amino]phenyl}pyridine-2-carboxylic acid;
6-methyl-5-{4-[(1-{[4-(propan-2-yl)phenyl]carbamoyl}-D-prolyl)amino]phenyl}pyridine-2-carboxylic acid;
3-methoxy-4′-[(1-{[4-(propan-2-yl)phenyl]carbamoyl}-D-prolyl)amino][1,1′-biphenyl]-4-carboxylic acid;
5-{4-[(1-{[3-fluoro-4-(propan-2-yl)phenyl]carbamoyl}-D-prolyl)amino]phenyl}-6-methylpyridine-2-carboxylic acid;
3-fluoro-4′-[(1-{[4-(propan-2-yl)phenyl]carbamoyl}-D-prolyl)amino][1,1′-biphenyl]-4-carboxylic acid;
4-{6-[(1-{[4-(trifluoromethyl)phenyl]carbamoyl}-D-prolyl)amino]pyridin-3-yl}benzoic acid;
4′-[(1-{[3-fluoro-4-(propan-2-yl)phenyl]carbamoyl}-D-prolyl)amino]-3-methoxy[1,1′-biphenyl]-4-carboxylic acid;
4-{3-fluoro-5-[(1-{[4-(propan-2-yl)phenyl]carbamoyl}-D-prolyl)amino]pyridin-2-yl}benzoic acid;
6-methyl-5-{4-[(1-{[3-methyl-4-(trifluoromethyl)phenyl]carbamoyl}-D-prolyl)amino]phenyl}pyridine-2-carboxylic acid;
4′-[(1-{[4-(trifluoromethyl)phenyl]carbamoyl}-DL-prolyl)amino][1,1′-biphenyl]-4-carboxylic acid;
5-{4-[(1-{[3-methyl-4-(trifluoromethyl)phenyl]carbamoyl}-DL-prolyl)amino]phenyl}pyridine-2-carboxylic acid;
4-{5-[(1-{[4-(propan-2-yl)phenyl]carbamoyl}-DL-prolyl)amino]pyridin-2-yl}benzoic acid;
4-{5-[(1-{[4-(trifluoromethyl)phenyl]carbamoyl}-DL-prolyl)amino]pyridin-2-yl}benzoic acid;
4′-({1-[(4-cyclobutylphenyl)carbamoyl]-DL-prolyl}amino)[1,1′-biphenyl]-4-carboxylic acid;
4-{5-fluoro-6-[(1-{[4-(trifluoromethyl)phenyl]carbamoyl}-DL-prolyl)amino]pyridin-3-yl}benzoic acid;
4-{5-fluoro-6-[(1-{[4-(propan-2-yl)phenyl]carbamoyl}-DL-prolyl)amino]pyridin-3-yl}benzoic acid;
4′-[(1-{[4-cyclopropyl-3-(trifluoromethyl)phenyl]carbamoyl}-DL-prolyl)amino][1,1′-biphenyl]-4-carboxylic acid;
2-methoxy-4′-[(1-{[4-(propan-2-yl)phenyl]carbamoyl}-DL-prolyl)amino][1,1′-biphenyl]-4-carboxylic acid;
3-methoxy-4′-[(1-{[3-methyl-4-(propan-2-yl)phenyl]carbamoyl}-DL-prolyl)amino][1,1′-biphenyl]-4-carboxylic acid;
4′-{[3-methyl-1-{[4-(propan-2-yl)phenyl]carbamoyl}-DL-prolyl]amino}[1,1′-biphenyl]-4-carboxylic acid;
4′-{[(trans)-3-methyl-1-{[4-(propan-2-yl)phenyl]carbamoyl}-DL-prolyl]amino}[1,1′-biphenyl]-4-carboxylic acid;
4′-{[(3-trans)-3-methyl-1-{[4-(propan-2-yl)phenyl]carbamoyl}-DL-prolyl]amino}[1,1′-biphenyl]-4-carboxylic acid;
4′-{[4-methoxy-1-{[4-(propan-2-yl)phenyl]carbamoyl}-DL-prolyl]amino}[1,1′-biphenyl]-4-carboxylic acid;
4′-{[(4-cis)-4-methoxy-1-{[4-(propan-2-yl)phenyl]carbamoyl}-DL-prolyl]amino}[1,1′-biphenyl]-4-carboxylic acid;
4′-{[1-({1-[4-(propan-2-yl)phenyl]ethyl}carbamoyl)-DL-prolyl]amino}[1,1′-biphenyl]-4-carboxylic acid;
3-[6-({1-[(4-cyclobutylphenyl)carbamoyl]-DL-prolyl}amino)pyridin-3-yl]benzoic acid;
4′-[(1-{[4-(trifluoromethyl)phenyl]carbamoyl}-D-prolyl)amino][1,1′-biphenyl]-4-carboxylic acid;
5-{4-[(1-{[3-methyl-4-(trifluoromethyl)phenyl]carbamoyl}-D-prolyl)amino]phenyl}pyridine-2-carboxylic acid;
4-{5-[(1-{[4-(propan-2-yl)phenyl]carbamoyl}-D-prolyl)amino]pyridin-2-yl}benzoic acid;
4-{5-[(1-{[4-(trifluoromethyl)phenyl]carbamoyl}-D-prolyl)amino]pyridin-2-yl}benzoic acid;
4′-({1-[(4-cyclobutylphenyl)carbamoyl]-D-prolyl}amino)[1,1′-biphenyl]-4-carboxylic acid;
4-{5-fluoro-6-[(1-{[4-(trifluoromethyl)phenyl]carbamoyl}-D-prolyl)amino]pyridin-3-yl}benzoic acid;
4-{5-fluoro-6-[(1-{[4-(propan-2-yl)phenyl]carbamoyl}-D-prolyl)amino]pyridin-3-yl}benzoic acid;
4′-[(1-{[4-cyclopropyl-3-(trifluoromethyl)phenyl]carbamoyl}-D-prolyl)amino][1,1′-biphenyl]-4-carboxylic acid;
2-methoxy-4′-[(1-{[4-(propan-2-yl)phenyl]carbamoyl}-D-prolyl)amino][1,1′-biphenyl]-4-carboxylic acid;
3-methoxy-4′-[(1-{[3-methyl-4-(propan-2-yl)phenyl]carbamoyl}-D-prolyl)amino][1,1′-biphenyl]-4-carboxylic acid;
4′-{[(3S)-3-methyl-1-{[4-(propan-2-yl)phenyl]carbamoyl}-D-prolyl]amino}[1,1′-biphenyl]-4-carboxylic acid;
4′-{[(3R)-3-methyl-1-{[4-(propan-2-yl)phenyl]carbamoyl}-L-prolyl]amino}[1,1′-biphenyl]-4-carboxylic acid;
4′-{[(3R)-3-methyl-1-{[4-(propan-2-yl)phenyl]carbamoyl}-D-prolyl]amino}[1,1′-biphenyl]-4-carboxylic acid;
4′-{[(3S)-3-methyl-1-{[4-(propan-2-yl)phenyl]carbamoyl}-L-prolyl]amino}[1,1′-biphenyl]-4-carboxylic acid;
4′-{[(4R)-4-methoxy-1-{[4-(propan-2-yl)phenyl]carbamoyl}-D-prolyl]amino}[1,1′-biphenyl]-4-carboxylic acid;
4′-{[1-({(1S)-1-[4-(propan-2-yl)phenyl]ethyl}carbamoyl)-D-prolyl]amino}[1,1′-biphenyl]-4-carboxylic acid; and
3-[6-({1-[(4-cyclobutylphenyl)carbamoyl]-D-prolyl}amino)pyridin-3-yl]benzoic acid,

or a pharmaceutically acceptable salt thereof.

Embodiment 179. The pharmaceutical composition of embodiments 176, wherein the GLP-1R agonist small molecule is selected from the group consisting of:
(S)-2-(4-(6-((4-chloro-2-fluorobenzyl)oxy)pyridin-2-yl)-3-fluorobenzyl)-1-(oxetan-2-ylmethyl)-1H-benzo[d]imidazole-6-carboxylic acid;
(S)-2-((3′-((4-chloro-2-fluorobenzyl)oxy)-2,4′-difluoro-[1,1′-biphenyl]-4-yl)methyl)-1-(oxetan-2-ylmethyl)-1H-benzo[d]imidazole-6-carboxylic acid;
(S)-2-(4-(4-(4-chloro-2-fluorobenzyloxy)-5-fluoropyrimidin-2-yl)-3-fluorobenzyl)-1-(oxetan-2-ylmethyl)-1H-benzo[d]imidazole-6-carboxylic acid;
(S)-2-((4-(3-(4-chloro-2-fluorobenzyloxy)isothiazol-4-yl)-5,6-dihydropyridin-1(2H)-yl)methyl)-1-(oxetan-2-ylmethyl)-1H-benzo[d]imidazole-6-carboxylic acid;
(S)-2-((1-(6-(4-cyano-2-fluorobenzyloxy)pyridin-2-yl)piperidin-4-ylidene)methyl)-3-(oxetan-2-ylmethyl)-3H-imidazo[4,5-b]pyridine-5-carboxylic acid;
(S)-2-(4-(6-((3,5-difluoropyridin-2-yl)methoxy)pyridin-2-yl)-2,5-difluorobenzyl)-3-(oxetan-2-ylmethyl)-3H-imidazo[4,5-b]pyridine-5-carboxylic acid;
(S)-2-(4-(4-((2,4-difluorobenzyl)oxy)-5-fluoropyrimidin-2-yl)-2-fluorobenzyl)-3-(oxetan-2-ylmethyl)-3H-imidazo[4,5-b]pyridine-5-carboxylic acid;
(S)-2-(4-(6-((4-cyano-2-fluorobenzyl)oxy)pyridin-2-yl)-2,5-difluorobenzyl)-1-(oxetan-2-ylmethyl)-1H-benzo[d]imidazole-6-carboxylic acid;
(S)-2-(2-chloro-4-(6-((4-chloro-2-fluorobenzyl)oxy)pyridin-2-yl)benzyl)-1-(oxetan-2-ylmethyl)-1H-benzo[d]imidazole-6-carboxylic acid;
(S)-2-((4-(2-((4-chloro-2-fluorobenzyl)oxy)oxazol-4-yl)-5,6-dihydropyridin-1(2H)-yl)methyl)-1-(oxetan-2-ylmethyl)-1H-benzo[d]imidazole-6-carboxylic acid; and
(S)-2-(4-(6-((4-chloro-2-fluorobenzyl)oxy)pyridin-2-yl)benzyl)-1-(oxetan-2-ylmethyl)-1H-benzo[d]imidazole-6-carboxylic acid,
   or a pharmaceutically acceptable salt thereof.

Embodiment 180. The pharmaceutical composition of embodiment 177, wherein the GLP-1R agonist small molecule is selected from the group consisting of:
2-[[4-[6-[(4-cyano-2-fluoro-phenyl)methoxy]-2-pyridyl]-3-methyl-phenyl]methyl]-3-[[oxetan-2-yl]methyl]benzimidazole-5-carboxylic acid;
2-[[4-[6-[(4-cyano-2-fluoro-phenyl)methoxy]-2-pyridyl]-2-fluoro-5-methyl-phenyl]methyl]-3-[[oxetan-2-yl]methyl]benzimidazole-5-carboxylic acid;
2-[[4-[6-[(4-Cyano-2-fluoro-phenyl)methoxy]-2-pyridyl]-2-fluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2,6-difluoro-phenyl]methyl]-3-[oxetan-2-ylmethyl]benzimidazole-5-carboxylic acid;
2-[[4-[6-[(4-Cyano-2-fluoro-phenyl)methoxy]-2-pyridyl]-2-fluoro-3-methyl-phenyl]methyl]-3-[[(2S)-oxetan-2-yl]methyl]benzimidazole-5-carboxylic acid; and
2-[[4-[6-[(4-Cyano-2-fluoro-phenyl)methoxy]-2-pyridyl]phenyl]methyl]-3-[[(2S)-oxetan-2-yl]methyl]benzimidazole-5-carboxylic acid, or a pharmaceutically acceptable salt thereof.

Embodiment 181. A pharmaceutical composition comprising:
a) a GIPR antagonist small molecule compound of Formula I:

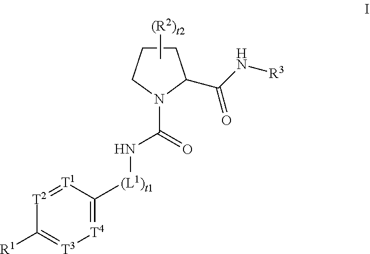

or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}:

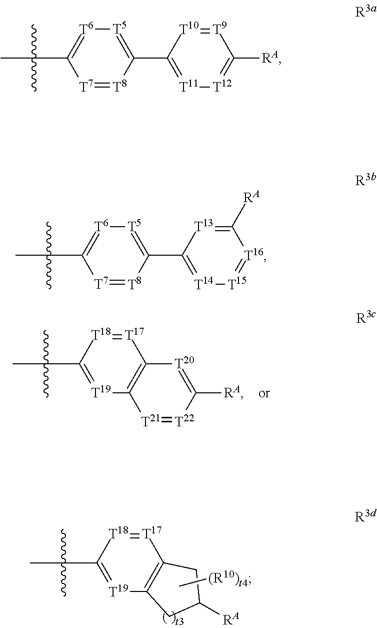

each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of the Formula:

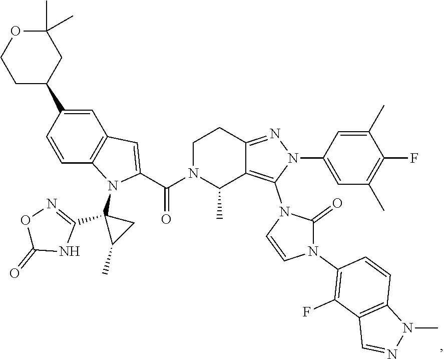

or a pharmaceutically acceptable salt thereof.

Embodiment 182. The pharmaceutical composition of any one of embodiments 176-181, wherein the pharmaceutical composition is formulated for oral administration.

Embodiment 183. The pharmaceutical composition of any one of embodiments 176-181, wherein the pharmaceutical composition is formulated for administration by subcutaneous injection.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

What is claimed is:

1. A method of treating a disease or condition comprising administering to a subject in need thereof a therapeutically effective amount of:
a) a GIPR antagonist small molecule compound of Formula I: 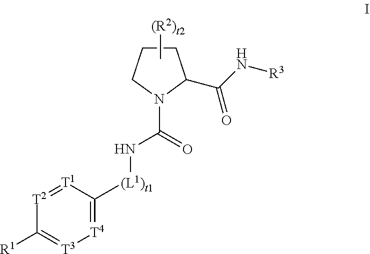
or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}: 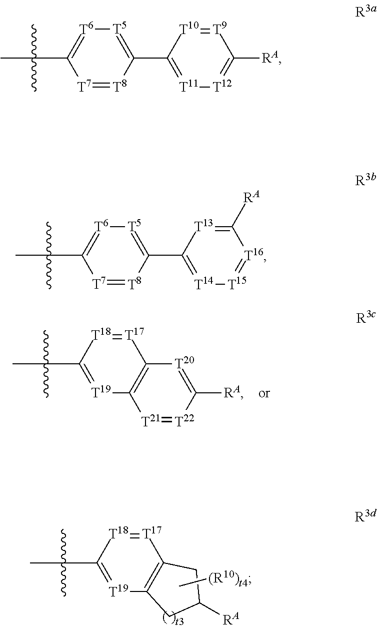
each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound or a pharmaceutically acceptable salt thereof,
wherein the disease or condition is selected from the group consisting of diabetes, hyperglycemia, insulin resistance, hepatic insulin resistance, impaired glucose tolerance, obesity, hyperlipidemia, hypertriglyceridemia, increased total cholesterol, increased low-density lipoprotein cholesterol, increased low high-density lipoprotein cholesterol, hyperinsulinemia, and cardiovascular disease.

2. The method of claim 1, wherein the condition is obesity.

3. The method of claim 1, wherein the condition is diabetes

4. The method of claim 1, wherein the therapeutically effective amount of the GIPR antagonist small molecule compound is from about 1 mg to about 100 mg.

5. The method of claim 1, wherein the GIPR antagonist small molecule compound or a pharmaceutically acceptable salt thereof is administered orally.

6. The method of claim 1, wherein the GIPR antagonist small molecule compound or a pharmaceutically acceptable salt thereof is administered by subcutaneous injection.

7. The method of claim 1, wherein the GIPR antagonist small molecule compound is selected from the group consisting of:
5-{4-[(1-{[4-(propan-2-yl)phenyl]carbamoyl}-D-prolyl)amino]phenyl}pyridine-2-carboxylic acid;
6-{4-[(1-{[4-(propan-2-yl)phenyl]carbamoyl}-D-prolyl)amino]phenyl}pyridine-3-carboxylic acid;
4′-[(1-{[4-(propan-2-yl)phenyl]carbamoyl}-D-prolyl)amino][1,1′-biphenyl]-3-carboxylic acid;
4′-[(1-{[4-(propan-2-yl)phenyl]carbamoyl}-D-prolyl)amino][1,1′-biphenyl]-4-carboxylic acid;
4-{6-[(1-{[4-(propan-2-yl)phenyl]carbamoyl}-D-prolyl)amino]pyridin-3-yl}benzoic acid;
3′-fluoro-4′-[(1-{[4-(propan-2-yl)phenyl]carbamoyl}-D-prolyl)amino][1,1′-biphenyl]-4-carboxylic acid;
4′-({1-[(4-cyclopropylphenyl)carbamoyl]-D-prolyl}amino)[1,1′-biphenyl]-4-carboxylic acid;
2-{4-[(1-{[4-(propan-2-yl)phenyl]carbamoyl}-D-prolyl)amino]phenyl}pyrimidine-5-carboxylic acid;
6-{4-[(1-{[4-(propan-2-yl)phenyl]carbamoyl}-D-prolyl)amino]phenyl}pyridine-2-carboxylic acid;
6-[(1-{[4-(propan-2-yl)phenyl]carbamoyl}-D-prolyl)amino]naphthalene-2-carboxylic acid;
8-methyl-6-[(1-{[4-(propan-2-yl)phenyl]carbamoyl}-D-prolyl)amino]quinoline-2-carboxylic acid;
4′-[(1-{[4-(prop-1-en-2-yl)phenyl]carbamoyl}-D-prolyl)amino][1,1′-biphenyl]-4-carboxylic acid;
4′-({1-[(4-chlorophenyl)carbamoyl]-D-prolyl}amino)[1,1′-biphenyl]-4-carboxylic acid;
4-{4-[(1-{[4-(propan-2-yl)phenyl]carbamoyl}-D-prolyl)amino]phenyl}pyridine-2-carboxylic acid; and
3′,5′-difluoro-4′-[(1-{[4-(propan-2-yl)phenyl]carbamoyl}-D-prolyl)amino][1,1′-biphenyl]-4-carboxylic acid,
or a pharmaceutically acceptable salt thereof.

8. The method of claim 1, wherein the GLP-1R agonist small molecule compound is a compound of Formula B-I: 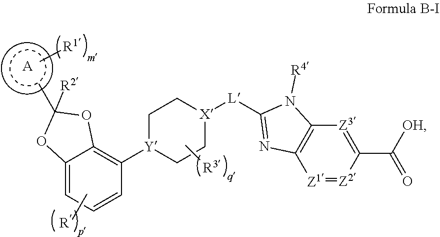
or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O}′, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}, and —N(R^{N}′)₂;
R^{5′} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
0 to 3 F atoms,
0 to 1 —CN, and
0 to 1 —OR^{O}′;
R^{6′} is a 5- to 6-membered heteroaryl, wherein said heteroaryl may be substituted with 0 to 2 substituents as valency allows independently selected from:
0 to 2 halogens,
0 to 1 substituent selected from —OR^{O′} and —N(R^{N}′)₂, and
0 to 2 —C₁₋₃alkyl, wherein the alkyl may be substituted with 0 to 3 substituents as valency allows independently selected from:
0 to 3 F atoms, and
0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z}′, or one of Z¹′, Z²′, and Z³′ is N and the other two are —CR^{Z}′; and
each R^{Z}′ is independently H, F, Cl, or —CH₃.

9. The method of claim 1, wherein the GLP-1R agonist small molecule compound is a compound of Formula C-I: 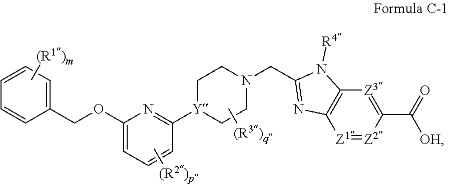
or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and
wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{6″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
0 to 3 F atoms,
0 to 1 —CN, and
0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
0 to 3 F atoms, and
0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
each R^{Z″} is independently H, F, Cl, or —CH₃.

10. A pharmaceutical composition comprising:
a) a GIPR antagonist small molecule compound of Formula I: 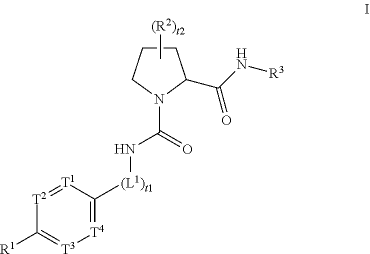
or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}: 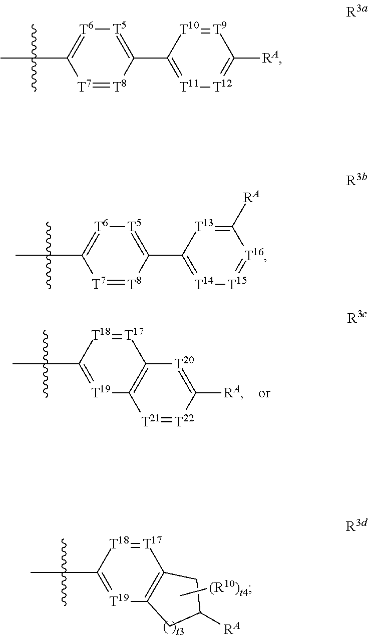
each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹ and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR⁸ or N, provided that only 0, 1, or 2 of T¹⁷, T¹⁸, and T¹⁹ can be N;
each R³ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula B-I: 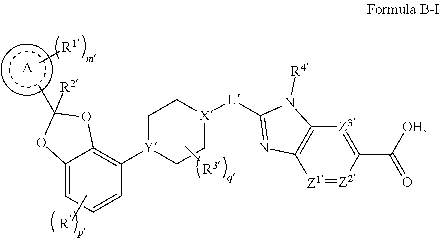
or a pharmaceutically acceptable salt thereof, wherein
R′ is F, Cl, or —CN;
p′ is 0 or 1;
ring A is phenyl or a 6-membered heteroaryl;
m′ is 0, 1, 2, or 3;
each R^{1′} is independently selected from halogen, —CN, —C₁₋₃alkyl, and —OC₁₋₃alkyl, wherein the alkyl of C₁₋₃alkyl and OC₁₋₃alkyl is substituted with 0 to 3 F atoms;
R^{2′} is H or —C₁₋₃alkyl, wherein alkyl is substituted with 0 to 1 OH;
each R^{3′} is independently F, —OH, —CN, —C₁₋₃alkyl, —OC₁₋₃alkyl, and —C₃₋₄cycloalkyl, or 2 R^{3′}s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q′ is 0, 1, or 2;
X′-L′ is N—CH₂, CHCH₂, or cyclopropyl;
Y′ is CH or N;
R^{4′} is —C₁₋₃alkyl, —C₀₋₃alkylene-C₃₋₆cycloalkyl, —C₀₋₃alkylene-R^{5′}, or —C₁₋₃alkylene-R^{6′}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O}, —SO₂—N(R^{N}′)₂, —C(O)—N(R^{N}′)₂, —N(C═O)(R^{N}′), and —N(R^{N}′)₂; wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O}, and —N(R^{N}′)₂;
R^{5′} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
0 to 3 F atoms,
0 to 1 —CN, and
0 to 1 —OR^{O}′;
R^{6′} is a 5- to 6-membered heteroaryl, wherein said heteroaryl may be substituted with 0 to 2 substituents as valency allows independently selected from:
0 to 2 halogens,
0 to 1 substituent selected from —OR^{O′} and —N(R^{N}′)₂, and
0 to 2 —C₁₋₃alkyl, wherein the alkyl may be substituted with 0 to 3 substituents as valency allows independently selected from:
0 to 3 F atoms, and
0 to 1 —OR^{O}′;
each R^{O}′ is independently H, or —C₁₋₃alkyl, wherein C₁₋₃alkyl may be substituted with 0 to 3 F atoms;
each R^{N}′ is independently H, or —C₁₋₃alkyl;
Z^{1′}, Z^{2′}, and Z^{3′} are each —CR^{Z}′, or one of Z¹′, Z²′, and Z³′ is N and the other two are —CR^{Z}′; and
each R^{Z}′ is independently H, F, Cl, or —CH₃.

11. A pharmaceutical composition comprising:
a) a GIPR antagonist small molecule compound of Formula I: 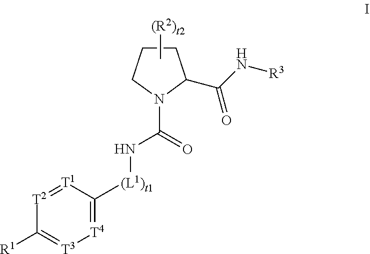
or a pharmaceutically acceptable salt thereof, wherein:
R¹ is H, halogen, —CN, C₁₋₈ alkyl, C₂₋₈ alkenyl, (C₃₋₆ cycloalkyl)-C₁₋₄ alkyl-, or C₃₋₆ cycloalkyl, wherein each of the C₁₋₈ alkyl, C₂₋₈ alkenyl, (C₃₋₆ cycloalkyl)-C₁₋₄ alkyl-, or C₃₋₆ cycloalkyl is optionally substituted with 1, 2, 3, 4, 5, or 6 substituents each independently selected from halogen, —OH, —CN, C₁₋₄ alkyl, C₁₋₄ haloalkyl, C₁₋₄ alkoxy, and C₁₋₄ haloalkoxy;
each R² is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
or two R², when attached to a same ring carbon atom of the proline ring in Formula I, together with the ring carbon atom to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
or two R², when attached to two adjacent ring carbon atoms of the proline ring in Formula I, together with the two ring carbon atoms to which they are attached, optionally form C₃₋₆ cycloalkyl or a 4- to 7-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
R³ is R^{3a}, R^{3b}, R^{3c}, or R^{3d}: 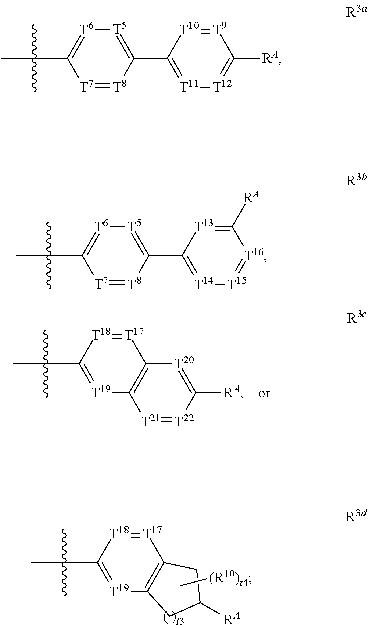
each of T¹, T², T³, and T⁴ is independently CR⁴ or N, provided that only 0, 1, or 2 of T¹, T², T³, and T⁴ can be N;
each R⁴ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁵, T⁶, T⁷, and T⁸ is independently CR⁵ or N, provided that only 0, 1, or 2 of T⁵, T⁶, T⁷, and T⁸ can be N;
each R⁵ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T⁹, T¹⁰, T¹¹, and T¹² is independently CR⁶ or N, provided that only 0, 1, or 2 of T⁹, T¹⁰, T¹¹, and T¹² can be N;
each R⁶ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹³, T¹⁴, T¹⁵, and T¹⁶ is independently CR⁷ or N, provided that only 0, 1, or 2 of T¹³, T¹⁴, T¹⁵, and T¹⁶ can be N;
each R⁷ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T¹⁷, T¹⁸, and T¹⁹ is independently CR³ or N, provided that only 0, 1, or 2 of T¹⁷, T¹⁸, and T¹⁹ can be N;
each R⁸ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each of T²⁰, T²¹, and T²² is independently CR⁹ or N, provided that only 0, 1, or 2 of T²⁰, T²¹ and T²² can be N;
each R⁹ is independently H, halogen, —CN, C₃₋₆ cycloalkyl, (C₃₋₆ cycloalkyl)-C₁₋₂ alkyl-, C₁₋₄ alkyl, C₁₋₄ cyanoalkyl, C₁₋₄ haloalkyl, C₁₋₄ alkoxy, or C₁₋₄ haloalkoxy;
each R¹⁰ is independently halogen, —OH, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R^{A} is —C(═O)—OH, 1H-tetrazol-5-yl, OH, —C(═O)—N(R¹¹)(R¹²), —C(═O)—OR¹³, 3-hydroxyisoxazol-5-yl, or —S(═O)₂NHCF₃;
each of R¹¹ and R¹² is independently H, C₁₋₆ alkyl, C₃₋₆ cycloalkyl, (C₃₋₆ cycloalkyl)-C₁₋₄ alkyl-, phenyl, or phenyl-C₁₋₄ alkyl-, wherein each of the C₁₋₆ alkyl, C₃₋₆ cycloalkyl, (C₃₋₆ cycloalkyl)-C₁₋₄ alkyl-, phenyl, or phenyl-C₁₋₄ alkyl- is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
or R¹¹ and R¹² together with the nitrogen atom to which they are attached form a 4- to 8-membered heterocycloalkyl optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-, wherein each of the C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl- is optionally substituted with 1, 2, or 3 substituents each independently selected from halogen, —OH, C₁₋₄ alkyl, C₁₋₄ haloalkyl, C₁₋₄ alkoxy, and C₁₋₄ haloalkoxy;
R¹³ is C₁₋₆ alkyl, C₃₋₆ cycloalkyl, (C₃₋₆ cycloalkyl)-C₁₋₄ alkyl-, phenyl, or phenyl-C₁₋₄ alkyl-, each of which is optionally substituted with 1, 2, 3, 4, or 5 substituents each independently selected from halogen, —OH, —CN, C₁₋₄ alkyl, C₁₋₄ haloalkyl, C₁₋₄ alkoxy, C₁₋₄ haloalkoxy, C₃₋₄ cycloalkyl, or (C₃₋₄ cycloalkyl)-C₁₋₄ alkyl-;
L¹ is C(R^{L})₂;
each R^{L} is independently H, C₁₋₂ alkyl, C₁₋₂ haloalkyl, C₁₋₂ alkoxy, or C₁₋₂ haloalkoxy;
or two R^{L} together with the carbon atom to which they are attached, optionally form C₃₋₆ cycloalkyl or a 3- to 6-membered heterocycloalkyl, each of which is optionally substituted with 1, 2, 3, or 4 substituents each independently selected from halogen, —OH, C₁₋₄ alkyl, C₁₋₄ haloalkyl, C₁₋₄ alkoxy, and C₁₋₄ haloalkoxy;
t1 is 0 or 1;
t2 is 0, 1, 2, 3, or 4;
t3 is 1 or 2; and
t4 is 0, 1, 2, 3, or 4; and
b) a glucagon-like peptide 1 receptor (GLP-1R) agonist small molecule compound of Formula C-I: 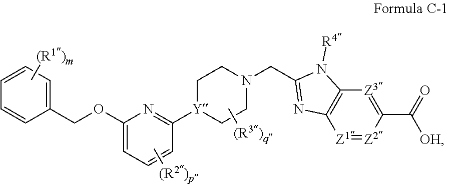
or a pharmaceutically acceptable salt thereof, wherein
each R^{1″} is independently halogen, —CN, —C₁₋₃alkyl, or —OC₁₋₃alkyl, wherein the alkyl of C₁₋₃alkyl and OC₁₋₃alkyl is substituted with 0 to 3 F atoms;
m″ is 0, 1, 2, or 3;
each R^{2″} is independently F, Cl, or —CN;
p″ is 0, 1 or 2;
each R^{3″} is independently F, —OH, —CN, —C₁₋₃alkyl, —OC₁₋₃alkyl, or —C₃₋₄cycloalkyl, or 2 R³s may together cyclize to form —C₃₋₄spirocycloalkyl, wherein the alkyl of C₁₋₃alkyl and OC₁₋₃alkyl, cycloalkyl, or spirocycloalkyl may be substituted as valency allows with 0 to 3 F atoms and with 0 to 1 —OH;
q″ is 0, 1, or 2;
Y″ is CH or N;
R^{4″} is —C₁₋₃alkyl, —C₀₋₃alkylene-C₃₋₆cycloalkyl, —C₀₋₃alkylene-R^{5″}, or —C₁₋₃alkylene-R^{6″}, wherein said alkyl may be substituted as valency allows with 0 to 3 substituents independently selected from 0 to 3 F atoms and 0 to 1 substituent selected from —C₀₋₁alkylene-CN, —C₀₋₁alkylene-OR^{O″}, and —N(R^{N″})₂, and
wherein said alkylene and cycloalkyl may be independently substituted as valency allows with 0 to 2 substituents independently selected from 0 to 2 F atoms and 0 to 1 substituent selected from —C₀₋₁alkylene-CN, —C₀₋₁alkylene-OR^{O″}, and —N(R^{N″})₂;
R^{5″} is a 4- to 6-membered heterocycloalkyl, wherein said heterocycloalkyl may be substituted with 0 to 2 substituents as valency allows independently selected from:
0 to 1 oxo (═O),
0 to 1 —CN,
0 to 2 F atoms, and
0 to 2 substituents independently selected from —C₁₋₃alkyl and —OC₁₋₃alkyl, wherein the alkyl of C₁₋₃alkyl and OC₁₋₃alkyl may be substituted with 0 to 3 substituents as valency allows independently selected from:
0 to 3 F atoms,
0 to 1 —CN, and
0 to 1 —OR^{O″};
R^{6″} is a 5- to 6-membered heteroaryl, wherein said heteroaryl may be substituted with 0 to 2 substituents as valency allows independently selected from:
0 to 2 halogens,
0 to 1 substituent selected from —OR^{O″} and —N(R^{N″})₂, and
0 to 2 —C₁₋₃alkyl, wherein the alkyl may be substituted with 0 to 3 substituents as valency allows independently selected from:
0 to 3 F atoms, and
0 to 1 —OR^{O″};
each R^{O″} is independently H, or —C₁₋₃alkyl, wherein C₁₋₃alkyl may be substituted with 0 to 3 F atoms;
each R^{N″} is independently H, or —C₁₋₃alkyl;
Z^{1″} is CH or N;
Z^{2″} and Z^{3″} are each independently —CR^{Z″} or N, provided that when Z^{1″} or Z^{3″} is N, Z^{2″} is —CR^{Z″}; and
each R^{Z″} is independently H, F, Cl, or —CH₃.
